(19) United States
(12) Patent Application Publication
(10) Pub. No.: US 2025/0248973 A1
(43) Pub. Date: Aug. 7, 2025

(54) CANNABINOID RECEPTOR 1 ANTAGONISTS/INVERSE AGONISTS AND USES THEREOF

(71) Applicant: Corbus Pharmaceuticals, Inc., Norwood, MA (US)

(72) Inventors: Marshall MORNINGSTAR, Framingham, MA (US); Zhuang JIN, Sharon, MA (US); Hongfeng DENG, Acton, MA (US)

(21) Appl. No.: 18/854,216

(22) PCT Filed: Apr. 7, 2023
(86) PCT No.: PCT/US2023/017817

§ 371 Date: Oct. 4, 2024

Related U.S. Application Data
(60) Provisional application No. 63/439,645, filed on Jan. 18, 2023.
(60) Provisional application No. 63/328,611, filed on Apr. 7, 2022.

(51) Int. Cl. A61K 31/415; A61K 31/4178; A61K 31/4192; A61K 31/4439; A61K 31/454; A61K 38/26; A61P 3/04; C07D 231/06; C07D 401/06; C07D 401/12; C07D 403/12

(52) U.S. Cl. CPC A61K 31/415; A61K 31/4178; A61K 31/4192; A61K 31/4439; A61K 31/454; A61K 38/26; A61P 3/04; C07D 231/06; C07D 401/06; C07D 401/12; C07D 403/12

(57) ABSTRACT

Disclosed herein are compounds suitable for use in the treatment of disorders, e.g., diabetic disorder, a dyslipidemia disorder, a cardiovascular disorder, an inflammatory disorder, a hepatic disorder, cancer, or obesity or co-morbidities thereof. Also disclosed are compositions containing one or more of the compounds and uses of the compounds in the treatment of disorders in a subject.

### BACKGROUND

Obesity is associated with an increase in the overall amount of adipose tissue (i.e., body fat), especially adipose tissue localized in the abdominal area. Obesity has reached epidemic proportions in the United States. The prevalence of obesity has steadily increased over the years among all racial and ethnic groups. The most recent data from the Centers for Disease Control and Prevention, and the National Center for Health Statistics report 66% of the adult population overweight (BMI, 25.0-29.9), 31% obese (BMI, 30-39.9), and 5% extremely obese (BMI, ≥40.0). Among children aged 6 through 19 years, 32% were overweight and 17% were obese. This translates to 124 million Americans medically overweight, and 44 million of these deemed obese. Obesity is responsible for more than 300,000 deaths annually, and will soon overtake tobacco usage as the primary cause of preventable death in the United States. Obesity is a chronic disease that contributes directly to numerous dangerous co-morbidities, including type 2 diabetes, cardiometabolic diseases, hepatic disorders, cardiovascular disease, inflammatory diseases, premature aging, and some forms of cancer. Type 2 diabetes, a serious and life-threatening disorder with growing prevalence in both adult and childhood populations, is currently the 7^{th} leading cause of death in the United States. Since more than 80% of patients with type 2 diabetes are overweight, obesity is the greatest risk factor for developing type 2 diabetes. Increasing clinical evidence indicates that the best way to control type 2 diabetes is to reduce weight. Accordingly, there is a continuing need for the development of improved medications that treat or prevent obesity.

The endocannabinoid system, comprised of the cannabinoid receptors (CB1 and CB₂) and their endogenous ligands (e.g., anandamide, 2-AG), plays a prominent role in the control of food intake and energy metabolism. CB1 receptors are widely expressed in the brain, including cortex, hippocampus, amygdala, pituitary and hypothalamus. CB1 receptors have also been identified in numerous peripheral organs and tissues, including thyroid gland, adrenal gland, reproductive organs, adipose tissue, liver, muscle, pancreas, kidney, and gastrointestinal tract. CB2 receptors are localized almost exclusively in immune and blood cells (Endocrine Reviews 2006, 27, 73).

The plant-derived cannabinoid agonist Δ⁹-tetrahydrocannabinol (Δ⁹-THC), the main psychoactive component of marijuana, binds to both CB1 and CB₂ receptors. Δ⁹-THC is widely reported to increase appetite and food intake (hyperphagia) in humans and in animals. This hyperphagic effect is largely blocked by pretreatment with selective CB1 receptor blockers (i.e., CB1 blockers)(e.g., rimonabant (SR141716A, Acomplia®)), strongly supporting the belief that CB1 receptor activation mediates the hyperphagic effect of Δ⁹-THC (Endocrine Reviews 2006, 27, 73).

The CB1 receptor is one of the most abundant and widely distributed G protein-coupled receptors in the mammalian brain. It is now known that the appetite-suppressant properties of CB1 antagonists can be mediated through either a direct action with CB1 receptors in brain regions associated with hunger and satiety (e.g., hypothalamus, mesolimbic regions), or a direct action with CB1 receptors in peripheral tissues (e.g., adipose tissue, kidney) [J. Clin Invest 2010, 120: 2953; Obesity 2011, 19: 1325]. However, CB1 receptors are far more broadly distributed in brain (e.g., neocortex, hippocampus, thalamus, cerebellum, and pituitary), and while interacting with targeted CB1 receptors in hypothalamus and mesolimbic regions to suppress appetite, CB1 antagonists have equal access to non-targeted CB1 receptors that have little if any role in appetite control. Binding to non-targeted receptors can often lead to unwanted side effects of CNS drugs (Endocrine Reviews 2006, 27: 73). For example, the CB1 blockers rimonabant and taranabant produce psychiatric and neurological side effects. These include depressed mood, anxiety, irritability, insomnia, dizziness, headache, seizures, and suicidality.

These side effects are dose-related and appear pronounced at the most efficacious weight-reducing doses of rimonabant and taranabant (JAMA 2006, 311, 323; Cell Metabolism 2008, 7, 68). The occurrence of therapeutic efficacy (appetite suppression) and side effects over the same dose range strongly suggest that both effects are mediated through concurrent antagonism of CB1 receptors in both ‘targeted’ and ‘non-targeted’ brain regions because brain-penetrant CB1 blockers do not selectively target CB1 receptors in the brain.

The beneficial effects of the CB1 antagonist rimonabant on body weight, adiposity, and diabetic and cardiometabolic risk factors such as high blood pressure, insulin resistance and blood lipids cannot be explained by weight loss derived from CNS-mediated appetite suppression alone (JAMA 2006, 311, 323). Approximately 50% of the benefit is likely derived from an interaction with CB1 receptors in peripheral tissues known to play an active role in metabolism. These include adipose tissue, liver, muscle, pancreas, kidney, and gastrointestinal tract.

Studies have identified 2 splice variants of the human CB1R mRNA, hCB1A (Rinaldi-Carmona et al., 1996; Shire et al., 1995) and hCB1B (Ryberg et al., 2005; Xiao et al., 2008). In particular, hCB1B has an internal deletion of 33 amino acids in the N terminus between Leu-21 and Gly-55 (Gonzalez-Mariscal, Krzysik-Walker, Doyle, et al., 2016). Further studies have demonstrated striking differences in the distribution of these splice variants (hCB1 and hCB1b) in human tissues (Gonzalez-Mariscal, Krzysik-Walker, Doyle, et al., 2016). Firstly, the hCB1B isoform was found to be highly expressed in human liver (where it is the dominant isoform) and pancreatic β-cells but not in brain. Secondly, expression in liver and β-cells was greatly increased in obese (BMI30.0) compared to non-obese individuals (BMI:30.0). As rimonabant bound to the two receptors (hCB1 and hCB1 b) with similar affinity (Gonzalez-Mariscal, Krzysik-Walker, Doyle, et al., 2016), the ability to more selectively target hCB1 b may bring additional benefits over compounds such as rimonabant.

Accordingly, there is a continual need to find effective and highly selective CB1 receptor blockers with limited or no CNS adverse side effects, including mood disorders. Particularly, it is desirable to find compounds that preferentially target CB1 b receptors in peripheral tissues (e.g., adipose tissue, liver, muscle, pancreas, and gastrointestinal tract), while sparing CB1 receptors in the brain.

### SUMMARY

The present disclosure provides novel pyrazoline compounds and pharmaceutically acceptable salts thereof that are cannabinoid 1 (CB1) receptor antagonists/inverse agonist, pharmaceutical compositions of such compounds, and the use of the compounds for the treatment of disorders mediated by the CB1 receptor.

In one aspect, the present disclosure provides a compound of formula (I):

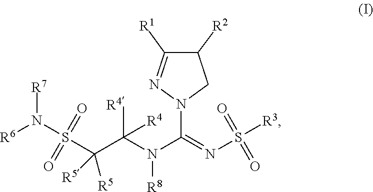

or a pharmaceutically acceptable salt thereof, in which R¹ is phenyl optionally substituted with one or more substituents selected from F, Cl, CN, and OCH₃; R² is C₁-C₆ alkyl or phenyl optionally substituted with F or CN; R³ is phenyl substituted with one or two substituents selected from F, Cl, CF₃, CN, OCH₃, C₂-C₆ alkynyl, and C(O)NH₂; or 5- or 6-membered heteroaryl containing 1-3 nitrogen atoms, in which the heteroaryl is optionally substituted with C₁-C₆ alkyl; R⁴, R^{4′}, R⁵, and R^{5′} are independently H or C₁-C₆ alkyl; R⁶ and R⁷ are independently H, OH, or C₁-C₆ alkyl; or R₆ and R₇, together with the nitrogen atom to which they are attached, form 5- or 6-membered heterocycloalkyl containing 1-2 nitrogen atoms and optionally substituted with C₁-C₆ alkyl; and R⁸ is H or CH₃, provided that at least one of the following is true: (i) R¹ is not 4-chlorophenyl or 4-methoxyphenyl; (ii) R² is C₁-C₆ alkyl or phenyl substituted with CN; (iii) R³ is not 4-chlorophenyl, 3-cyanophenyl, or 3-methoxyphenyl; (iv) one of R⁴ and R⁵ is C₁-C₆ alkyl; (v) one of R⁶ and R⁷ is not H; and (vi) R⁸ is CH₃.

In some embodiments, the compound is a compound of formula (IA):

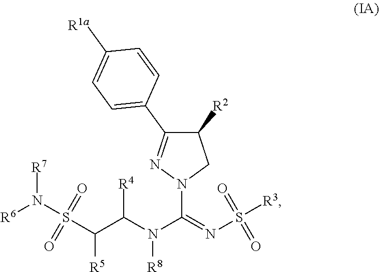

or a pharmaceutically acceptable salt thereof, in which R^{1a} is F, Cl, CN, or OCH₃.

In some embodiments, the compound is a compound of formula (IIA):

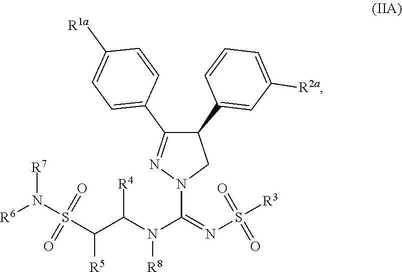

or a pharmaceutically acceptable salt thereof, in which R^{1a} is F, Cl, CN, or OCH₃, and R^{2a} is H or CN. In some embodiments, R^{2a} is H. In other embodiments, R^{2a} is CN.

In some embodiments, the compound is a compound of formula (IIIA):

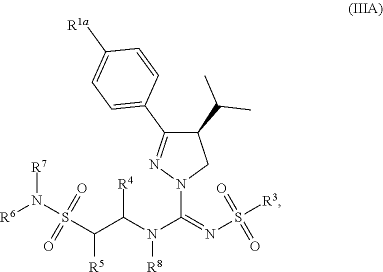

or a pharmaceutically acceptable salt thereof, in which R^{1a} is F, Cl, CN, or OCH₃.

In some embodiments, the compound is a compound of formula (IB):

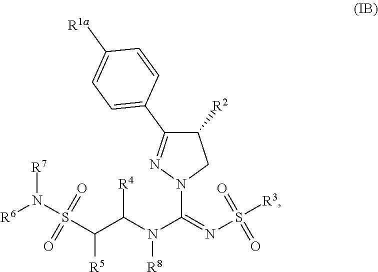

or a pharmaceutically acceptable salt thereof, in which R^{1a} is F, Cl, CN, or OCH₃.

In some embodiments, the compound is a compound of formula (IIB):

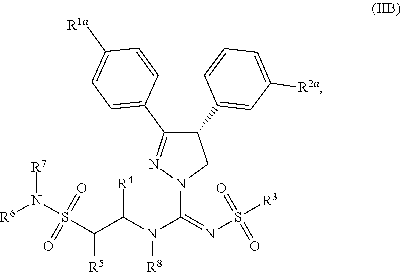

or a pharmaceutically acceptable salt thereof, in which R^{1a} is F, Cl, CN, or OCH₃, and R^{2a} is H or CN. In some embodiments, R^{2a} is H. In other embodiments, R^{2a} is CN.

In some embodiments, the compound is a compound of formula (IIIB):

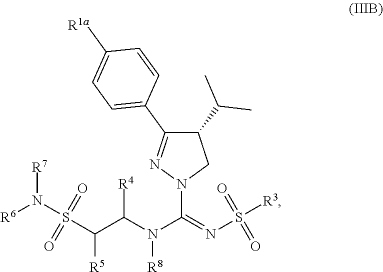

or a pharmaceutically acceptable salt thereof, in which R^{1a} is F, Cl, CN, or OCH₃.

In some embodiments of any of the aspects described herein (e.g., formulas (IA), (IB), (IIA), (IIB), (IIIA), and (IIIB)), R^{1a} is F.

In some embodiments of any of the aspects described herein (e.g., formulas (IA), (IB), (IIA), (IIB), (IIIA), and (IIIB)), R^{1a} is Cl.

In some embodiments of any of the aspects described herein (e.g., formulas(IA), (IB), (IIA), (IIB), (IIIA), and (IIIB)), R^{1a} is OCH₃.

In some embodiments of any of the aspects described herein (e.g., formulas (I), (IA), (IB), (IIA), (IIB), (IIIA), and (IIIB)), R³ is phenyl substituted with one or two groups selected from F, Cl, CF₃, CN, OCH₃, C₂-C₆ alkynyl, or C(O)NH₂. For example, R³ is:

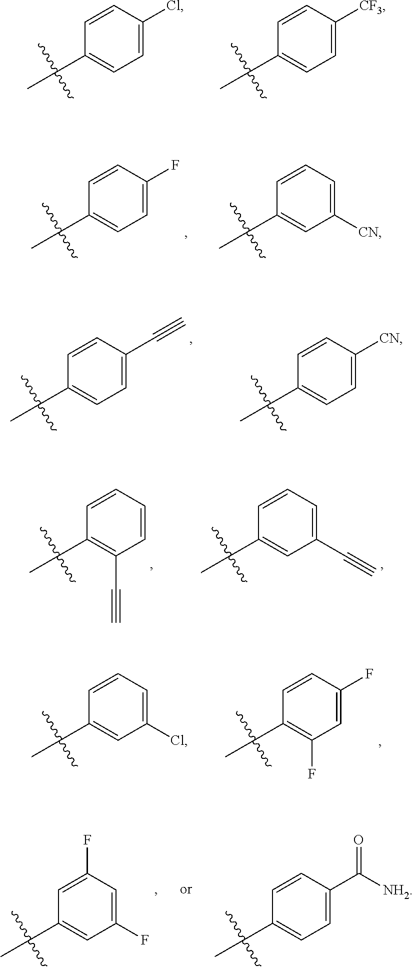

In some embodiments, R³ is,

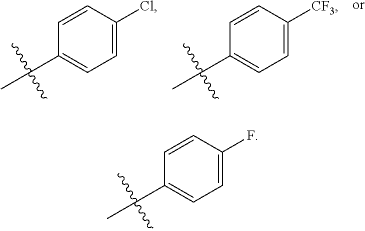 [SMILES: *c1ccc(Cl)cc1 ; *c1ccc(C(F)(F)F)cc1 ; "or" ; *c1ccc(F)cc1]

In some embodiments of any of the aspects described herein (e.g., formulas (I), (IA), (IB), (IIA), (IIB), (IIIA), and (IIIB)), R³ is 5- or 6-membered heteroaryl containing 1-3 nitrogen atoms, in which the heteroaryl is optionally substituted with C₁-C₆ alkyl. For example, R³ is:

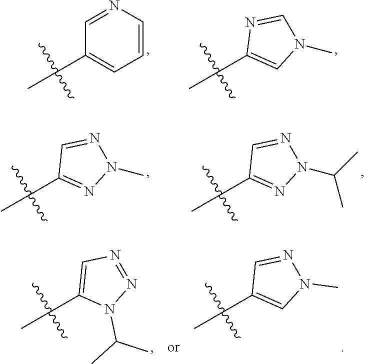

In some embodiments of any of the aspects described herein (e.g., formulas (I), (IA), (IB), (IIA), (IIB), (IIIA), and (IIIB)), R⁴ is H.

In some embodiments of any of the aspects described herein (e.g., formulas (I), (IA), (IB), (IIA), (IIB), (IIIA), and (IIIB)), R⁴ is C₁-C₆ alkyl, e.g., methyl or isopropyl.

In some embodiments of any of the aspects described herein (e.g., formulas (I), (IA), (IB), (IIA), (IIB), (IIIA), and (IIIB)), R⁵ is H.

In some embodiments of any of the aspects described herein (e.g., formulas (I), (IA), (IB), (IIA), (IIB), (IIIA), and (IIIB)), R⁵ is C₁-C₆ alkyl, e.g., methyl or isopropyl.

In some embodiments of any of the aspects described herein (e.g., formulas (I), (IA), (IB), (IIA), (IIB), (IIIA), and (IIIB)), R⁶ is H.

In some embodiments of any of the aspects described herein (e.g., formulas (I), (IA), (IB), (IIA), (IIB), (IIIA), and (IIIB)), R⁶ is C₁-C₆ alkyl, e.g., R⁶ is methyl, ethyl, or isopropyl.

In some embodiments of any of the aspects described herein (e.g., formulas (I), (IA), (IB), (IIA), (IIB), (IIIA), and (IIIB)), R⁶ is OH.

In some embodiments of any of the aspects described herein (e.g., formulas (I), (IA), (IB), (IIA), (IIB), (IIIA), and (IIIB)), R⁷ is H.

In some embodiments of any of the aspects described herein (e.g., formulas (I), (IA), (IB), (IIA), (IIB), (IIIA), and (IIIB)), R⁷ is C₁-C₆ alkyl, e.g., methyl, ethyl, or isopropyl.

In some embodiments of any of the aspects described herein (e.g., formulas (I), (IA), (IB), (IIA), (IIB), (IIIA), and (IIIB)), R⁷ is OH.

In some embodiments of any of the aspects described herein (e.g., formulas (I), (IA), (IB), (IIA), (IIB), (IIIA), and (IIIB)), R⁶ and R⁷, together with the nitrogen atom to which they are attached, form 5- or 6-membered heterocycloalkyl containing 1-2 nitrogen atoms and optionally substituted with C₁-C₆ alkyl, e.g.,

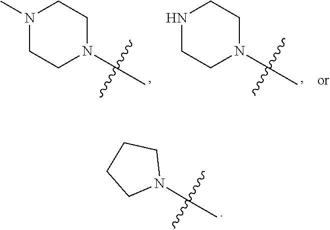

In some embodiments of any of the aspects described herein (e.g., formulas (I), (IA), (IB), (IIA), (IIB), (IIIA), and (IIIB)), R⁸ is H.

In some embodiments of any of the aspects described herein (e.g., formulas (I), (IA), (IB), (IIA), (IIB), (IIIA), and (IIIB)), R⁸ is CH₃.

In some embodiments, the compound is selected from any of compounds 1-86, 103-110, 155-172, 175-182 of Table 1, or a pharmaceutically acceptable salt thereof.

In another aspect, the present disclosure provides a compound of formula (II):

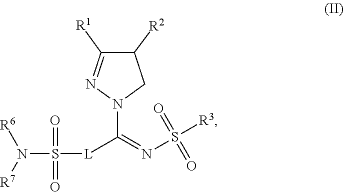

or a pharmaceutically acceptable salt thereof, in which R¹ is phenyl optionally substituted with one or more substituents selected from halogen, C₁-C₆ alkoxy, and CN; R² is C₁-C₆ alkyl, 5- or 6-membered heteroaryl, or phenyl optionally substituted with halogen or CN; R³ is phenyl substituted with one or more substituents selected from halogen, C₁-C₆ alkoxy, CF₃, CN, C₂-C₆ alkynyl, and C(O)NH₂; or 5- or 6-membered heteroaryl containing 1-3 nitrogen atoms, in which the heteroaryl is optionally substituted with C₁-C₆ alkyl; R⁶ and R⁷ are independently H, OH, or C₁-C₆ alkyl; or R₆ and R₇, together with the nitrogen atom to which they are attached, form 5- or 6-membered heterocycloalkyl containing 1-2 nitrogen atoms and optionally substituted with C₁-C₆ alkyl; L is

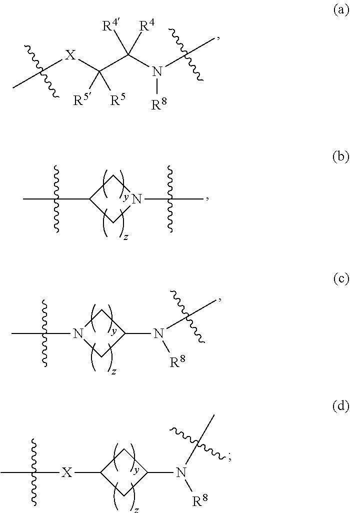

R⁸ is H or C₁-C₆ alkyl; R⁴, R^{4′}, R⁵, and R^{5′} are independently H or C₁-C₆ alkyl optionally substituted with C₁-C₆ alkoxy; X is O or NR⁹; R⁹ is H or C₁-C₆ alkyl; and y and z are independently 1, 2, or 3.

In some embodiments, the compound is a compound of formula (IVA):

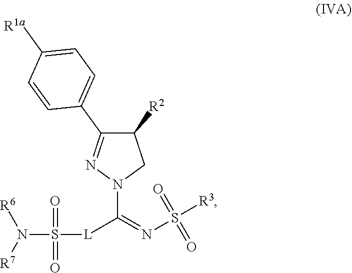

or a pharmaceutically acceptable salt thereof, in which R^{1a} is halogen, CN, or C₁-C₆ alkoxy.

In some embodiments, the compound is a compound of formula (VA):

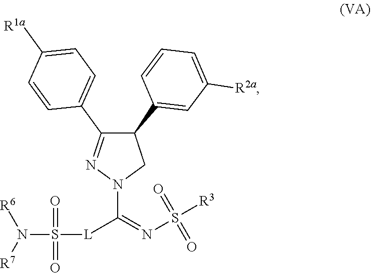

or a pharmaceutically acceptable salt thereof, in which R^{1a} is halogen, CN, or C₁-C₆ alkoxy, and R^{2a} is H halogen, C₁-C₆ alkoxy, or CN. In some embodiments, R^{2a} is H. In other embodiments, R^{2a} is CN.

In some embodiments, the compound is a compound of formula (VIA):

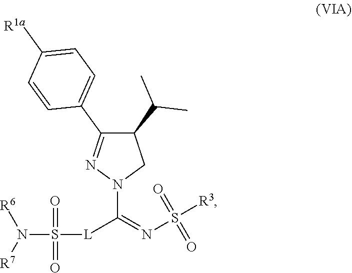

or a pharmaceutically acceptable salt thereof, in which R^{1a} is halogen, CN, or C₁-C₆ alkoxy.

In some embodiments, the compound is a compound of formula (IVB):

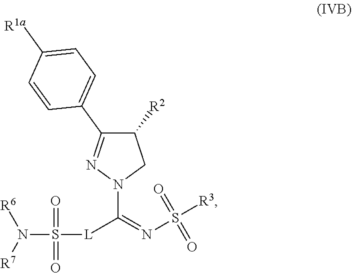

or a pharmaceutically acceptable salt thereof, in which R^{1a} is halogen, CN, or C₁-C₆ alkoxy.

In some embodiments, the compound is a compound of formula (VB):

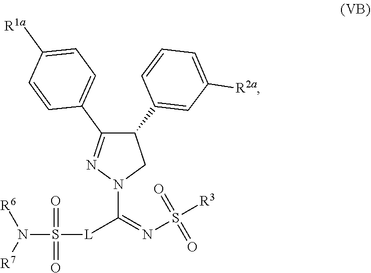

or a pharmaceutically acceptable salt thereof, in which R^{1a} is halogen, CN, or C₁-C₆ alkoxy, and R^{2a} is H, halogen, C₁-C₆ alkoxy, or CN. In some embodiments, R^{2a} is H. In other embodiments, R^{2a} is CN.

In some embodiments, the compound is a compound of formula (VIB):

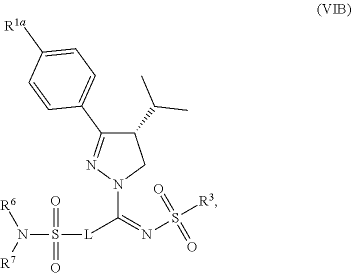

or a pharmaceutically acceptable salt thereof, in which R^{1a} is halogen, CN, or C₁-C₆ alkoxy.

In some embodiments of any of the aspects described herein (e.g., formulas (IVA), (IVB), (VA), (VB), (VIA), and (VIB)), R^{1a} is F.

In some embodiments of any of the aspects described herein (e.g., formulas (IVA), (IVB), (VA), (VB), (VIA), and (VIB)), R^{1a} is Cl.

In some embodiments of any of the aspects described herein (e.g., formulas (IVA), (IVB), (VA), (VB), (VIA), and (VIB)), R^{1a} is OCH₃.

In some embodiments of any of the aspects described herein (e.g., formulas (II), (IVA), (IVB), (VA), (VB), (VIA), and (VIB)), R³ is phenyl substituted with one or two groups selected from halogen, CF₃, CN, C₁-C₆ alkoxy, C₂-C₆ alkynyl, or C(O)NH₂. For example, R³ is:

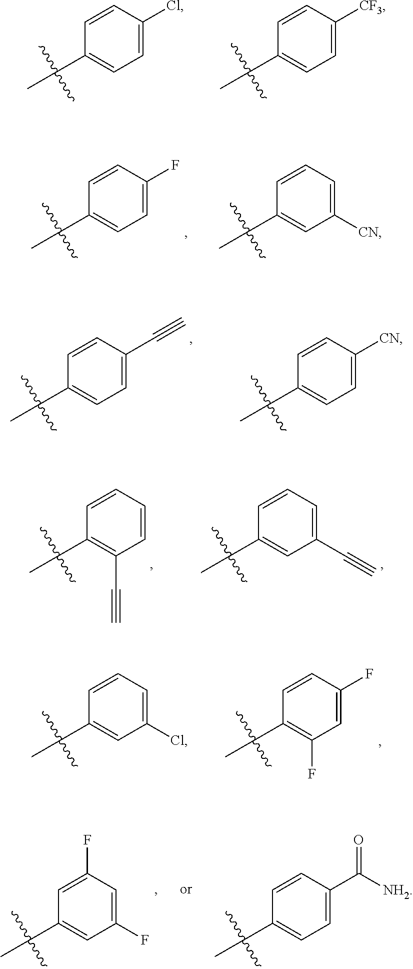

In some embodiments, R³ is

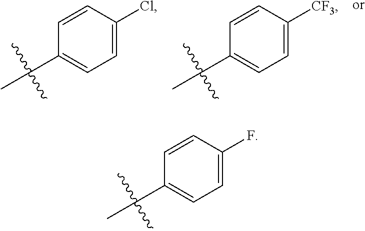 [SMILES: *c1ccc(Cl)cc1 ; *c1ccc(C(F)(F)F)cc1 ; "or" ; *c1ccc(F)cc1]

In some embodiments of any of the aspects described herein (e.g., formulas (II), (IVA), (IVB), (VA), (VB), (VIA), and (VIB)), R³ is 5- or 6-membered heteroaryl containing 1-3 nitrogen atoms, in which the heteroaryl is optionally substituted with C₁-C₆ alkyl. For example, R³ is:

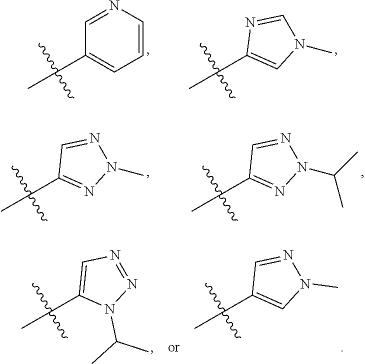

In some embodiments of any of the aspects described herein (e.g., formulas (II), (IVA), (IVB), (VA), (VB), (VIA), and (VIB)), R⁴ is H.

In some embodiments of any of the aspects described herein (e.g., formulas (II), (IVA), (IVB), (VA), (VB), (VIA), and (VIB)), R⁴ is C₁-C₆ alkyl, e.g., methyl or isopropyl.

In some embodiments of any of the aspects described herein (e.g., formulas (II), (IVA), (IVB), (VA), (VB), (VIA), and (VIB)), R⁵ is H.

In some embodiments of any of the aspects described herein (e.g., formulas (II), (IVA), (IVB), (VA), (VB), (VIA), and (VIB)), R⁵ is C₁-C₆ alkyl, e.g., methyl or isopropyl.

In some embodiments of any of the aspects described herein (e.g., formulas (II), (IVA), (IVB), (VA), (VB), (VIA), and (VIB)), R⁶ is H.

In some embodiments of any of the aspects described herein (e.g., formulas (II), (IVA), (IVB), (VA), (VB), (VIA), and (VIB)), R⁶ is C₁-C₆ alkyl, e.g., R⁶ is methyl, ethyl, or isopropyl.

In some embodiments of any of the aspects described herein (e.g., formulas (II), (IVA), (IVB), (VA), (VB), (VIA), and (VIB)), R⁶ is OH.

In some embodiments of any of the aspects described herein (e.g., formulas (II), (IVA), (IVB), (VA), (VB), (VIA), and (VIB)), R⁷ is H.

In some embodiments of any of the aspects described herein (e.g., formulas (II), (IVA), (IVB), (VA), (VB), (VIA), and (VIB)), R⁷ is C₁-C₆ alkyl, e.g., methyl, ethyl, or isopropyl.

In some embodiments of any of the aspects described herein (e.g., formulas (II), (IVA), (IVB), (VA), (VB), (VIA), and (VIB)), R⁷ is OH.

In some embodiments of any of the aspects described herein (e.g., formulas(II), (IVA), (IVB), (VA), (VB), (VIA), and (VIB)), R⁶ and R⁷, together with the nitrogen atom to which they are attached, form 5- or 6-membered heterocycloalkyl containing 1-2 nitrogen atoms and optionally substituted with C₁-C₆ alkyl, e.g.,

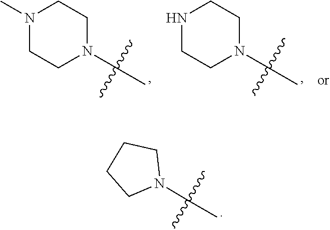

In some embodiments of any of the aspects described herein (e.g., formulas (II), (IVA), (IVB), (VA), (VB), (VIA), and (VIB)), R⁸ is H.

In some embodiments of any of the aspects described herein (e.g., formulas (II), (IVA), (IVB), (VA), (VB), (VIA), and (VIB)), R⁸ is CH₃.

In some embodiments, the compound is a selected from any of compounds 99-102, 111-154, 173-174, 183-250 of Table 1, or a pharmaceutically acceptable salt thereof.

In another aspect, the present disclosure provides a compound of formula (III):

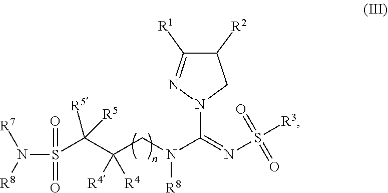

or a pharmaceutically acceptable salt thereof, in which R¹ is phenyl optionally substituted with one or more substituents selected from F, Cl, CN, and OCH₃; R² is C₁-C₆ alkyl, 5- or 6-membered heteroaryl, or phenyl optionally substituted with F or CN; R³ is phenyl substituted with one or more substituents selected from F, Cl, CF₃, CN, OCH₃, C₂-C₆ alkynyl, and C(O)NH₂; or 5- or 6-membered heteroaryl containing 1-3 nitrogen atoms, in which the heteroaryl is optionally substituted with C₁-C₆ alkyl; R⁴, R^{4′}, R⁵, and R^{5′} are independently H or C₁-C₆ alkyl; R⁶ and R⁷ are independently H, OH, or C₁-C₆ alkyl; or R₆ and R₇, together with the nitrogen atom to which they are attached, form 5- or 6-membered heterocycloalkyl containing 1-2 nitrogen atoms and optionally substituted with C₁-C₆ alkyl; R⁸ is H or C₁-C₆ alkyl; and n is 1, 2, or 3.

In some embodiments, the compound is a selected from any of compounds 87-98 of Table 1, or a pharmaceutically acceptable salt thereof.

In another aspect, the present disclosure provides a pharmaceutical composition including any one of the compounds described herein (e.g., any one of the compounds of formula (I), (IA), (IB), (IIA), (IIB), (IIIA), (IIIB), (II), (IVA), (VA), (VIA), (IVB), (VB), (VIB), and (III), and Table 1) or a pharmaceutically acceptable salt thereof, and a pharmaceutically acceptable excipient.

In another aspect, the present disclosure provides a method of treating a disease, which includes administering to a subject in need thereof a therapeutically effective amount of any one of the compounds described herein (e.g., any one of the compounds of formula (I), (IA), (IB), (IIA), (IIB), (IIIA), (IIIB), (II), (IVA), (VA), (VIA), (IVB), (VB), (VIB), and (III), and Table 1) or a pharmaceutically acceptable salt thereof, in which the disease is a diabetic disorder, a dyslipidemia disorder, a cardiovascular disorder, an inflammatory disorder, a hepatic disorder, or cancer.

In some embodiments, the disease is a diabetic disorder, e.g., Type 1 diabetes, Type 2 diabetes, inadequate glucose tolerance, or insulin resistance.

In some embodiments, the disease is a dyslipidemia disorder, e.g., undesirable blood lipid levels, low levels of high-density lipoprotein, high levels of low-density lipoprotein, high levels of triglycerides, or a combination thereof.

In some embodiments, the disease is a cardiovascular disorder, e.g., atherosclerosis, hypertension, stroke, or heart attack.

In some embodiments, the disease is an inflammatory disorder, e.g., osteoarthritis, rheumatoid arthritis, inflammatory bowel diseases, or obesity-associated inflammation.

In some embodiments, the disease is a hepatic disorder, e.g., liver inflammation, liver fibrosis, non-alcoholic steatohepatitis, fatty liver, enlarged liver, alcoholic liver disease, jaundice, cirrhosis, or hepatitis.

In some embodiments, the disease is cancer, e.g., colon cancer, breast cancer, thyroid cancer, alveolar rhabdomyosarcoma, or hepatocellular carcinoma.

In another aspect, the present disclosure provides a method of treating obesity or a co-morbidity of obesity, which includes administering to a subject in need thereof a therapeutically effective amount of any one of the compounds described herein (e.g., any one of the compounds of formula (I), (IA), (IB), (IIA), (IIB), (IIIA), (IIIB), (II), (IVA), (VA), (VIA), (IVB), (VB), (VIB), and (Ill), and Table 1) or a pharmaceutically acceptable salt thereof.

In some embodiments, the co-morbidity of obesity is diabetes, dyslipidemia, Metabolic Syndrome, dementia, a cardiovascular disease, or a hepatic disease.

In some embodiments, the co-morbidity of obesity is hypertension; gallbladder disease; gastrointestinal disorders; menstrual irregularities; degenerative arthritis; venous statis ulcers; pulmonary hypoventilation syndrome; sleep apnea; snoring; coronary artery disease; arterial sclerotic disease; pseudotumor cerebri; accident proneness; increased risks with surgeries; osteoarthritis; high cholesterol; or increased incidence of malignancy of the ovaries, cervix, uterus, breasts, prostrate, or gallbladder.

In another aspect, the present disclosure provides a method of reversing adipose tissue deposition in a subject, which includes administering to a subject in need thereof a therapeutically effective amount of any one of the compounds described herein (e.g., any one of the compounds of formula (I), (IA), (IB), (IIA), (IIB), (IIIA), (IIIB), (II), (IVA), (VA), (VIA), (IVB), (VB), (VIB), and (Ill), and Table 1) or a pharmaceutically acceptable salt thereof.

In some embodiments of any of the methods described herein, the method further includes administering to the subject a second therapeutic agent, e.g., a PPAR-γ agonist, a biguanide, insulin or an insulin mimetic, a sulfonylurea, an α-glucosidase inhibitor, an HMG-CoA reductase inhibitor, a sequestrant, nicotinyl alcohol, nicotinic acid or a salt thereof, a PPAR-α agonist, an inhibitor of cholesterol absorption, an acyl CoA:cholesterol acyltransferase inhibitor, probucol, a PPAR-α/γ agonist, an ileal bile acid transporter inhibitor, an insulin receptor activator, a dipeptidyl peptidase IV inhibitor, exenatide, pramlintide, an FBPase inhibitor, a glucagon receptor antagonist, glucagon-like peptide 1,a glucagon-like peptide 1 receptor agonist, a growth hormone secretagogue, a growth hormone secretagogue receptor agonist, a growth hormone secretagogue receptor antagonist, a melanocortin agonist, a melanocortin 4 receptor agonist, a beta-3 agonist, a serotonin receptor 2C agonist, an orexin antagonist, a melanin concentrating hormone 1 antagonist, a melanin concentrating hormone 2 agonist, a melanin concentrating hormone 2 antagonist, a galanin antagonist, a CCK agonist, a CCK-A agonist, a corticotropin-releasing hormone agonist, an NPY 5 antagonist, an NPY 1 antagonist, a histamine receptor-3 modulator, a histamine receptor-3 blocker, a p-hydroxy steroid dehydrogenase-1 inhibitor, a phosphodiesterase inhibitor, a phosphodiesterase-3B inhibitor, a norepinephrine transport inhibitor, a non-selective serotonin/norepinephrine transport inhibitor, a ghrelin antagonist, a leptin derivative, a bombesin receptor subtype 3 agonist, a ciliary neurotrophic factor or a derivative thereof, a monoamine reuptake inhibitor, an uncoupling protein-1 activator, an uncoupling protein-2 activator, an uncoupling protein-3 activator, a thyroid hormone beta agonist, a fatty acid synthase inhibitor, a diacylglycerol acetyltransferase 2 inhibitor, an acetyl-CoA carboxylase-2 inhibitor, a glucocorticoid antagonist, an acyl-estrogen, a lipase inhibitor, a fatty acid transporter inhibitor, a dicarboxylate transporter inhibitor, a glucose transporter inhibitor, a sodium-glucose co-transporter, a phosphate transporter inhibitor, a serotonin reuptake inhibitor, a thiazolidinedione, Metformin, Topiramate, an opiate antagonist, a non-selective transport inhibitor, or a MAO inhibitor. In some embodiments, the second therapeutic agent is a glucagon-like peptide 1 receptor agonist (e.g., liraglutide, semaglutide, exenatide, lixisenatide, dulaglutide, or tirzepatide).

In some embodiments of any of the methods described herein, the subject is a human.

In another aspect, the present disclosure discloses a use of any one of the compounds described herein (e.g., any one of the compounds of formula (I), (IA), (IB), (IIA), (IIB), (IIIA), (IIIB), (II), (IVA), (VA), (VIA), (IVB), (VB), (VIB), and (Ill), and Table 1) or a pharmaceutically acceptable salt thereof in the manufacture of a medicament for use in any one of the methods disclosed herein.

In another aspect, the present disclosure provides a compound (e.g., any one of the compounds of formulas (I), (IA), (IB), (IIA), (IIB), (IIIA), (IIIB), (II), (IVA), (VA), (VIA), (IVB), (VB), (VIB), and (III), and Table 1) for use in any one of the methods disclosed herein.

TABLE 1

| Compound | Structure |
|---|---|
| 1 | 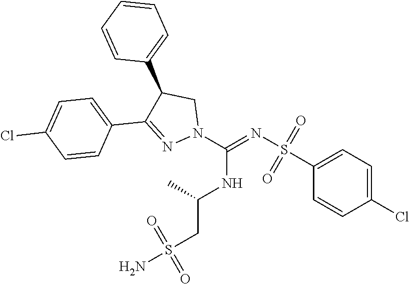 [SMILES: C[C@@H](CS(N)(=O)=O)N/C(=N\S(=O)(=O)c1ccc(Cl)cc1)N1C[C@H](c2ccccc2)C(c2ccc(Cl)cc2)=N1] |
| 2 | 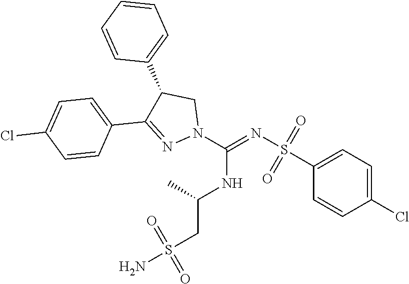 [SMILES: C[C@@H](CS(N)(=O)=O)N/C(=N\S(=O)(=O)c1ccc(Cl)cc1)N1C[C@@H](c2ccccc2)C(c2ccc(Cl)cc2)=N1] |
| 3 | 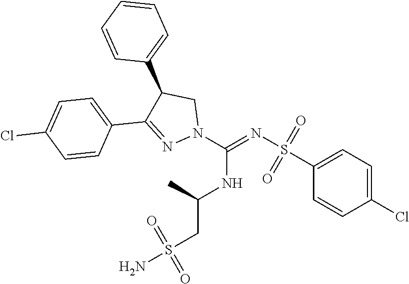 [SMILES: C[C@H](CS(N)(=O)=O)N/C(=N\S(=O)(=O)c1ccc(Cl)cc1)N1C[C@H](c2ccccc2)C(c2ccc(Cl)cc2)=N1] |
| 4 | 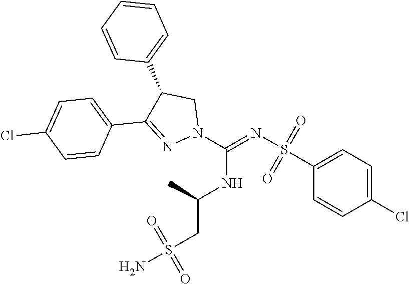 [SMILES: C[C@H](CS(N)(=O)=O)N/C(=N\S(=O)(=O)c1ccc(Cl)cc1)N1C[C@@H](c2ccccc2)C(c2ccc(Cl)cc2)=N1] |
| 5 | 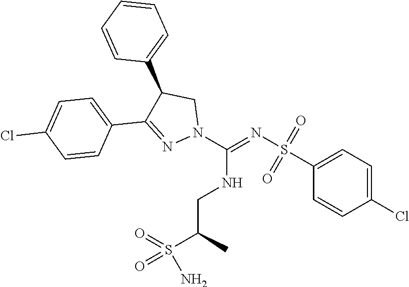 [SMILES: C[C@H](CN/C(=N\S(=O)(=O)c1ccc(Cl)cc1)N1C[C@H](c2ccccc2)C(c2ccc(Cl)cc2)=N1)S(N)(=O)=O] |
| 6 | 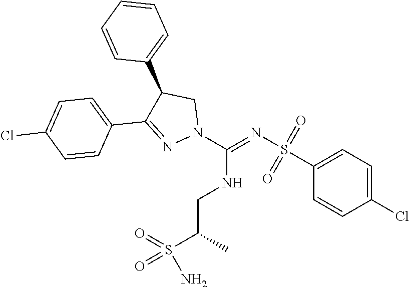 [SMILES: C[C@@H](CN/C(=N\S(=O)(=O)c1ccc(Cl)cc1)N1C[C@H](c2ccccc2)C(c2ccc(Cl)cc2)=N1)S(N)(=O)=O] |
| 7 | 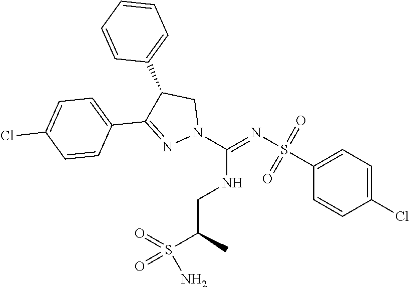 [SMILES: C[C@H](CN/C(=N\S(=O)(=O)c1ccc(Cl)cc1)N1C[C@@H](c2ccccc2)C(c2ccc(Cl)cc2)=N1)S(N)(=O)=O] |
| 8 | 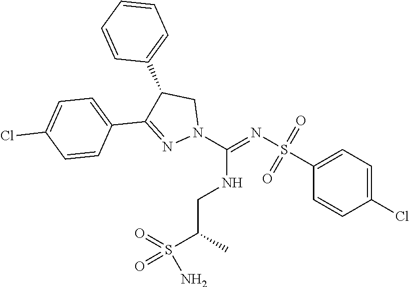 [SMILES: C[C@@H](CN/C(=N\S(=O)(=O)c1ccc(Cl)cc1)N1C[C@@H](c2ccccc2)C(c2ccc(Cl)cc2)=N1)S(N)(=O)=O] |
| 9 | 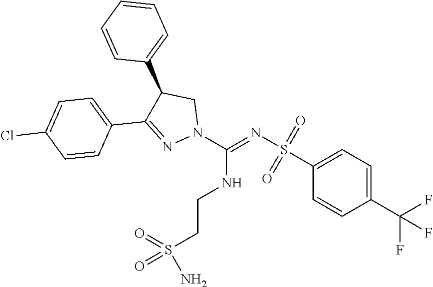 [SMILES: NS(=O)(=O)CCN/C(=N\S(=O)(=O)c1ccc(C(F)(F)F)cc1)N1C[C@H](c2ccccc2)C(c2ccc(Cl)cc2)=N1] |
| 10 | 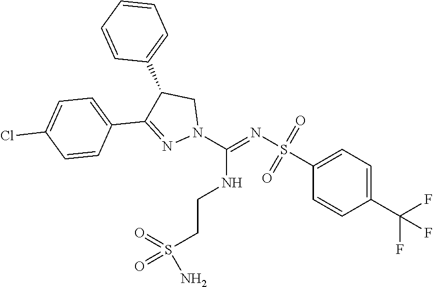 [SMILES: NS(=O)(=O)CCN/C(=N\S(=O)(=O)c1ccc(C(F)(F)F)cc1)N1C[C@@H](c2ccccc2)C(c2ccc(Cl)cc2)=N1] |
| 11 | 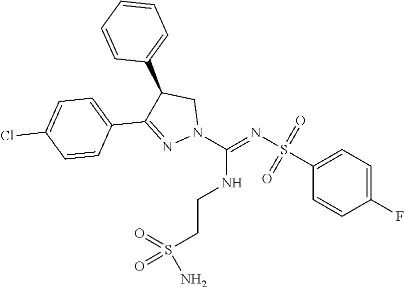 [SMILES: NS(=O)(=O)CCN/C(=N\S(=O)(=O)c1ccc(F)cc1)N1C[C@H](c2ccccc2)C(c2ccc(Cl)cc2)=N1] |
| 12 | 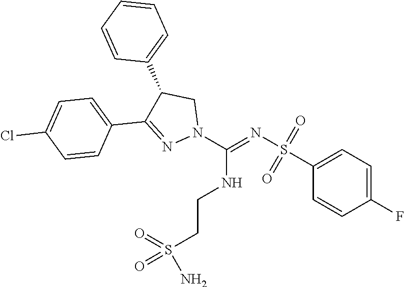 [SMILES: NS(=O)(=O)CCN/C(=N\S(=O)(=O)c1ccc(F)cc1)N1C[C@@H](c2ccccc2)C(c2ccc(Cl)cc2)=N1] |
| 13 | 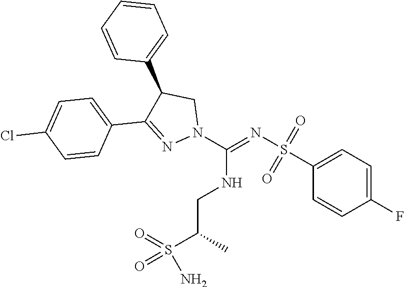 [SMILES: C[C@@H](CN/C(=N\S(=O)(=O)c1ccc(F)cc1)N1C[C@H](c2ccccc2)C(c2ccc(Cl)cc2)=N1)S(N)(=O)=O] |
| 14 | 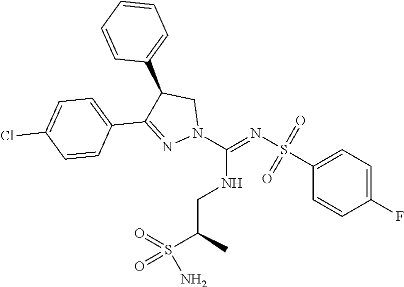 [SMILES: C[C@H](CN/C(=N\S(=O)(=O)c1ccc(F)cc1)N1C[C@H](c2ccccc2)C(c2ccc(Cl)cc2)=N1)S(N)(=O)=O] |
| 15 | 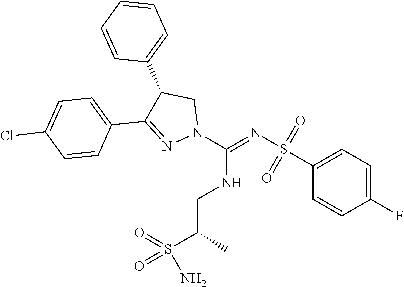 [SMILES: C[C@@H](CN/C(=N\S(=O)(=O)c1ccc(F)cc1)N1C[C@@H](c2ccccc2)C(c2ccc(Cl)cc2)=N1)S(N)(=O)=O] |
| 16 | 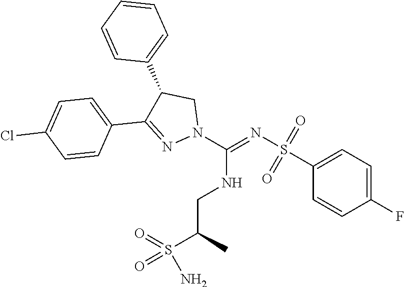 [SMILES: C[C@H](CN/C(=N\S(=O)(=O)c1ccc(F)cc1)N1C[C@@H](c2ccccc2)C(c2ccc(Cl)cc2)=N1)S(N)(=O)=O] |
| 17 | 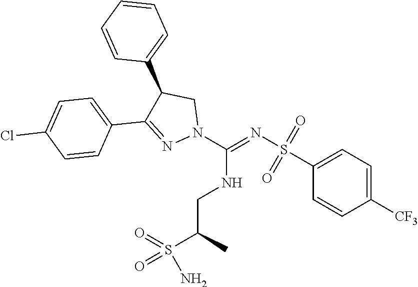 [SMILES: C[C@H](CN/C(=N\S(=O)(=O)c1ccc(C(F)(F)F)cc1)N1C[C@H](c2ccccc2)C(c2ccc(Cl)cc2)=N1)S(N)(=O)=O] |
| 18 | 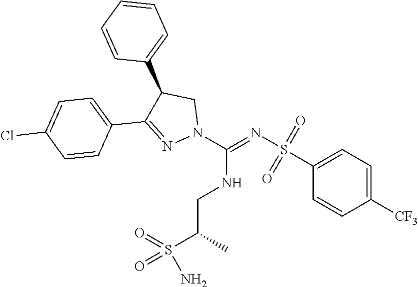 [SMILES: C[C@@H](CN/C(=N\S(=O)(=O)c1ccc(C(F)(F)F)cc1)N1C[C@H](c2ccccc2)C(c2ccc(Cl)cc2)=N1)S(N)(=O)=O] |
| 19 | 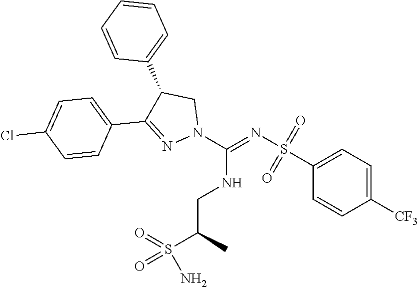 [SMILES: C[C@H](CN/C(=N\S(=O)(=O)c1ccc(C(F)(F)F)cc1)N1C[C@@H](c2ccccc2)C(c2ccc(Cl)cc2)=N1)S(N)(=O)=O] |
| 20 | 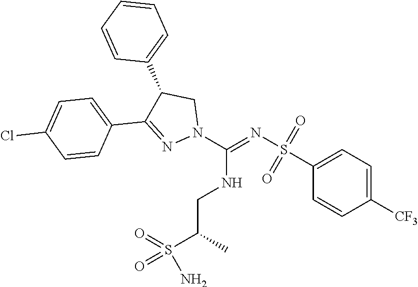 [SMILES: C[C@@H](CN/C(=N\S(=O)(=O)c1ccc(C(F)(F)F)cc1)N1C[C@@H](c2ccccc2)C(c2ccc(Cl)cc2)=N1)S(N)(=O)=O] |
| 21 | 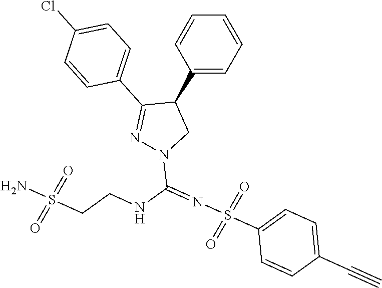 [SMILES: C#Cc1ccc(S(=O)(=O)N=C(NCCS(N)(=O)=O)N2C[C@H](c3ccccc3)C(c3ccc(Cl)cc3)=N2)cc1] |
| 22 | 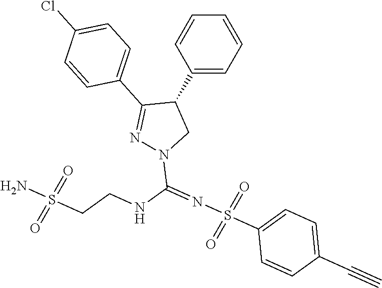 [SMILES: C#Cc1ccc(S(=O)(=O)N=C(NCCS(N)(=O)=O)N2C[C@@H](c3ccccc3)C(c3ccc(Cl)cc3)=N2)cc1] |
| 23 | 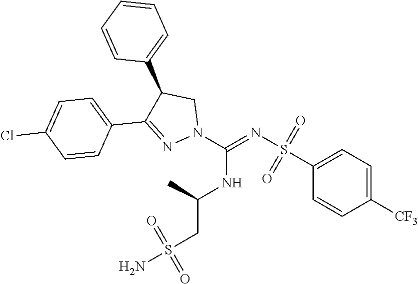 [SMILES: C[C@H](CS(N)(=O)=O)N/C(=N\S(=O)(=O)c1ccc(C(F)(F)F)cc1)N1C[C@H](c2ccccc2)C(c2ccc(Cl)cc2)=N1] |
| 24 | 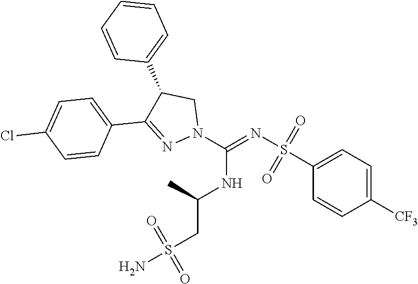 [SMILES: C[C@H](CS(N)(=O)=O)N/C(=N\S(=O)(=O)c1ccc(C(F)(F)F)cc1)N1C[C@@H](c2ccccc2)C(c2ccc(Cl)cc2)=N1] |
| 25 | 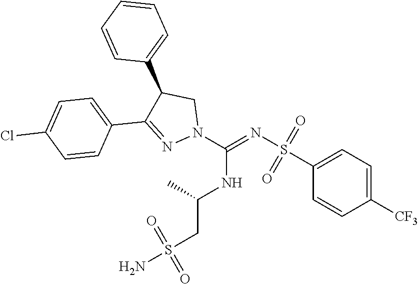 [SMILES: C[C@@H](CS(N)(=O)=O)N/C(=N\S(=O)(=O)c1ccc(C(F)(F)F)cc1)N1C[C@H](c2ccccc2)C(c2ccc(Cl)cc2)=N1] |
| 26 | 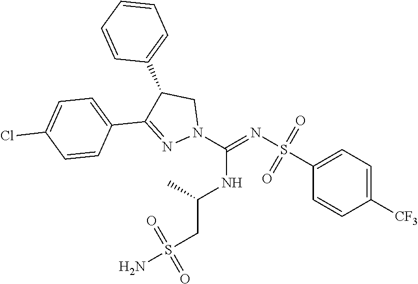 [SMILES: C[C@@H](CS(N)(=O)=O)N/C(=N\S(=O)(=O)c1ccc(C(F)(F)F)cc1)N1C[C@@H](c2ccccc2)C(c2ccc(Cl)cc2)=N1] |
| 27 | 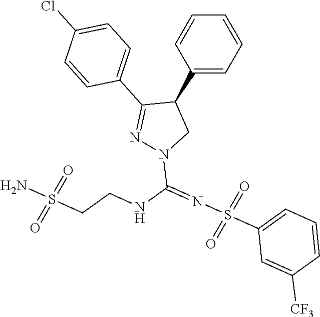 [SMILES: NS(=O)(=O)CCNC(=NS(=O)(=O)c1cccc(C(F)(F)F)c1)N1C[C@H](c2ccccc2)C(c2ccc(Cl)cc2)=N1] |
| 28 | 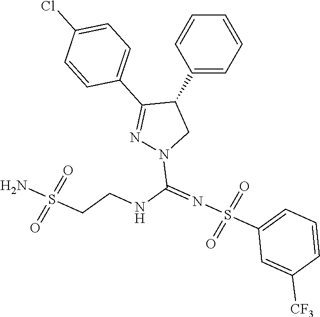 [SMILES: NS(=O)(=O)CCNC(=NS(=O)(=O)c1cccc(C(F)(F)F)c1)N1C[C@@H](c2ccccc2)C(c2ccc(Cl)cc2)=N1] |
| 29 | 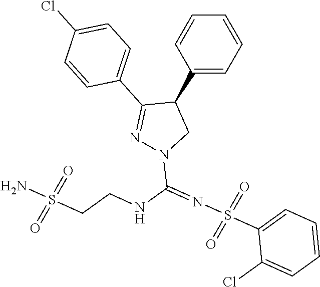 [SMILES: NS(=O)(=O)CCNC(=NS(=O)(=O)c1ccccc1Cl)N1C[C@H](c2ccccc2)C(c2ccc(Cl)cc2)=N1] |
| 30 | 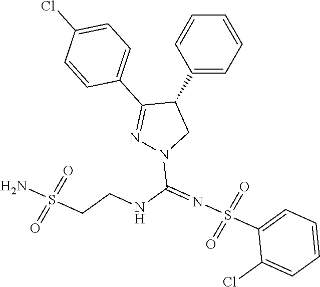 [SMILES: NS(=O)(=O)CCNC(=NS(=O)(=O)c1ccccc1Cl)N1C[C@@H](c2ccccc2)C(c2ccc(Cl)cc2)=N1] |
| 31 | 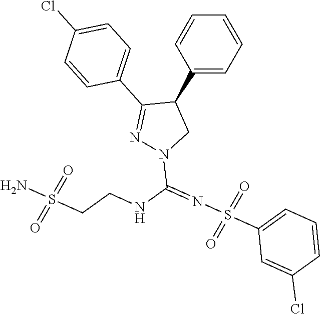 [SMILES: NS(=O)(=O)CCNC(=NS(=O)(=O)c1cccc(Cl)c1)N1C[C@H](c2ccccc2)C(c2ccc(Cl)cc2)=N1] |
| 32 | 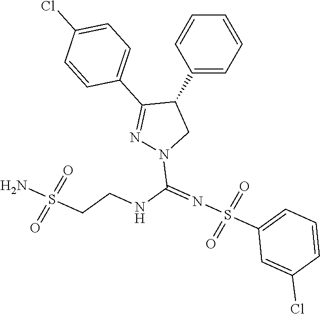 [SMILES: NS(=O)(=O)CCNC(=NS(=O)(=O)c1cccc(Cl)c1)N1C[C@@H](c2ccccc2)C(c2ccc(Cl)cc2)=N1] |
| 33 | 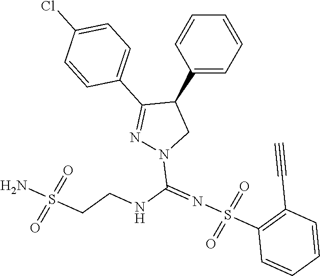 [SMILES: C#Cc1ccccc1S(=O)(=O)N=C(NCCS(N)(=O)=O)N1C[C@H](c2ccccc2)C(c2ccc(Cl)cc2)=N1] |
| 34 | 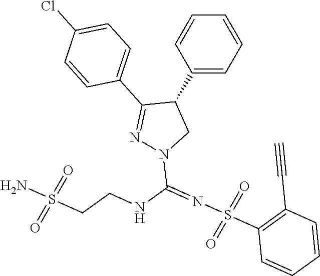 [SMILES: C#Cc1ccccc1S(=O)(=O)N=C(NCCS(N)(=O)=O)N1C[C@@H](c2ccccc2)C(c2ccc(Cl)cc2)=N1] |
| 35 | 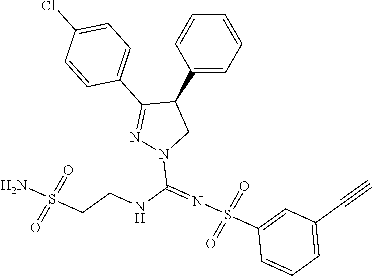 [SMILES: C#Cc1cccc(S(=O)(=O)N=C(NCCS(N)(=O)=O)N2C[C@H](c3ccccc3)C(c3ccc(Cl)cc3)=N2)c1] |
| 36 | 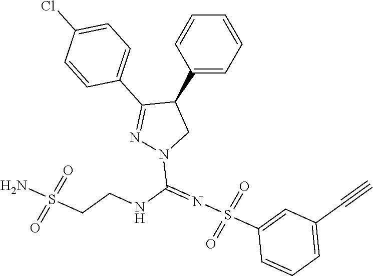 [SMILES: C#Cc1cccc(S(=O)(=O)N=C(NCCS(N)(=O)=O)N2C[C@H](c3ccccc3)C(c3ccc(Cl)cc3)=N2)c1] |
| 37 | 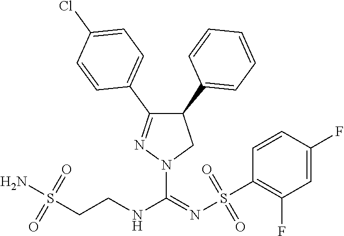 [SMILES: NS(=O)(=O)CCN/C(=N/S(=O)(=O)c1ccc(F)cc1F)N1C[C@H](c2ccccc2)C(c2ccc(Cl)cc2)=N1] |
| 38 | 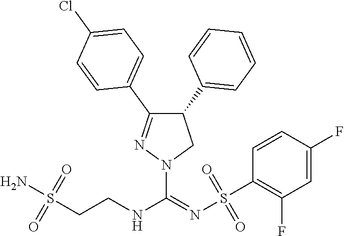 [SMILES: NS(=O)(=O)CCN/C(=N/S(=O)(=O)c1ccc(F)cc1F)N1C[C@@H](c2ccccc2)C(c2ccc(Cl)cc2)=N1] |
| 39 | 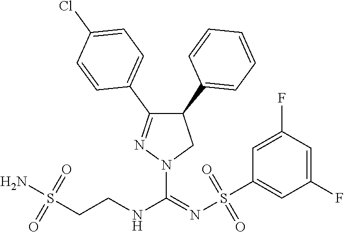 [SMILES: NS(=O)(=O)CCN/C(=N/S(=O)(=O)c1cc(F)cc(F)c1)N1C[C@H](c2ccccc2)C(c2ccc(Cl)cc2)=N1] |
| 40 | 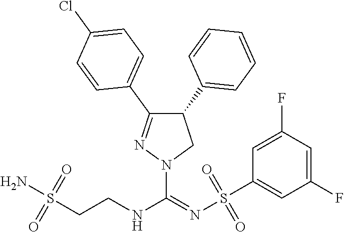 [SMILES: NS(=O)(=O)CCN/C(=N/S(=O)(=O)c1cc(F)cc(F)c1)N1C[C@@H](c2ccccc2)C(c2ccc(Cl)cc2)=N1] |
| 41 | 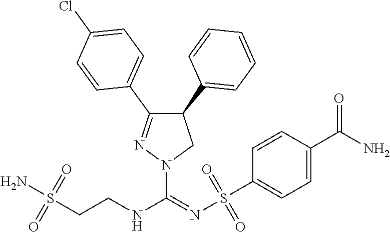 [SMILES: NC(=O)c1ccc(S(=O)(=O)/N=C(/NCCS(N)(=O)=O)N2C[C@H](c3ccccc3)C(c3ccc(Cl)cc3)=N2)cc1] |
| 42 | 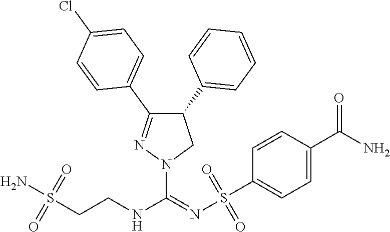 [SMILES: NC(=O)c1ccc(S(=O)(=O)/N=C(/NCCS(N)(=O)=O)N2C[C@@H](c3ccccc3)C(c3ccc(Cl)cc3)=N2)cc1] |
| 43 | 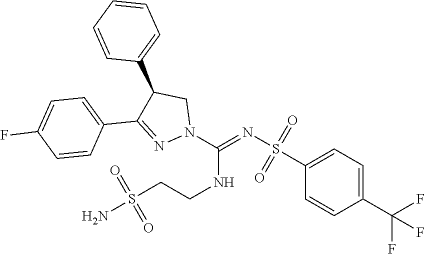 [SMILES: NS(=O)(=O)CCN/C(=N\S(=O)(=O)c1ccc(C(F)(F)F)cc1)N1C[C@H](c2ccccc2)C(c2ccc(F)cc2)=N1] |
| 44 | 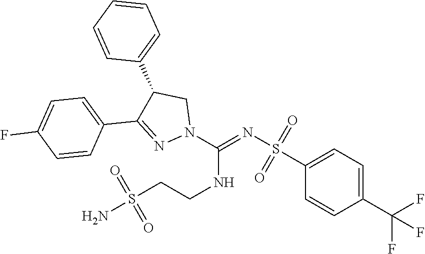 [SMILES: NS(=O)(=O)CCN/C(=N\S(=O)(=O)c1ccc(C(F)(F)F)cc1)N1C[C@@H](c2ccccc2)C(c2ccc(F)cc2)=N1] |
| 45 | 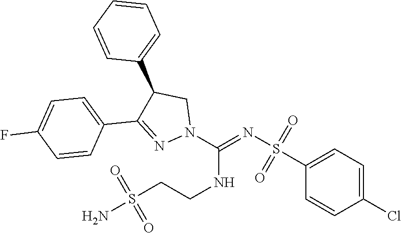 [SMILES: NS(=O)(=O)CCN/C(=N\S(=O)(=O)c1ccc(Cl)cc1)N1C[C@H](c2ccccc2)C(c2ccc(F)cc2)=N1] |
| 46 | 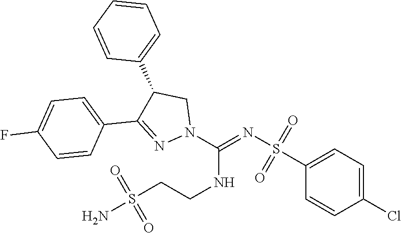 [SMILES: NS(=O)(=O)CCN/C(=N\S(=O)(=O)c1ccc(Cl)cc1)N1C[C@@H](c2ccccc2)C(c2ccc(F)cc2)=N1] |
| 47 | 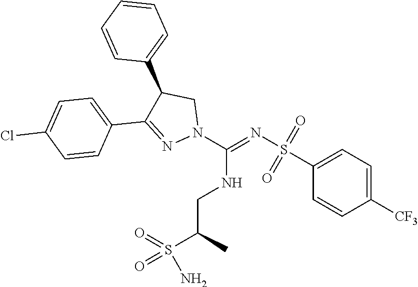 [SMILES: C[C@H](CN/C(=N\S(=O)(=O)c1ccc(C(F)(F)F)cc1)N1C[C@H](c2ccccc2)C(c2ccc(Cl)cc2)=N1)S(N)(=O)=O] |
| 48 | 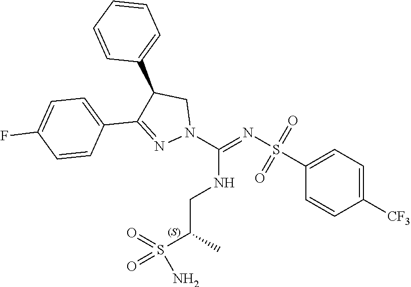 |
| 49 | 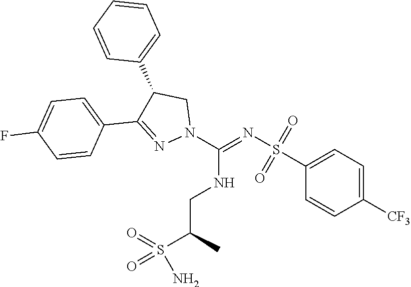 [SMILES: C[C@H](CN/C(=N\S(=O)(=O)c1ccc(C(F)(F)F)cc1)N1C[C@@H](c2ccccc2)C(c2ccc(F)cc2)=N1)S(N)(=O)=O] |
| 50 | 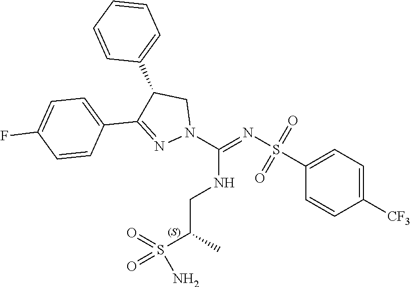 |
| 51 | 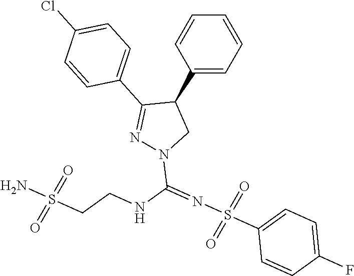 [SMILES: NS(=O)(=O)CCNC(=NS(=O)(=O)c1ccc(F)cc1)N1C[C@H](c2ccccc2)C(c2ccc(Cl)cc2)=N1] |
| 52 | 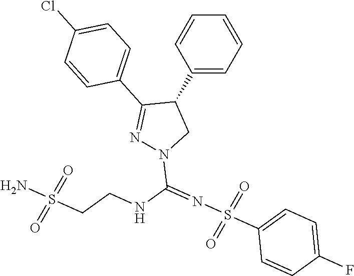 [SMILES: NS(=O)(=O)CCNC(=NS(=O)(=O)c1ccc(F)cc1)N1C[C@@H](c2ccccc2)C(c2ccc(Cl)cc2)=N1] |
| 53 | 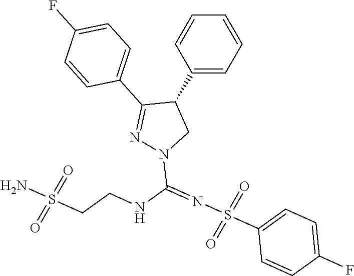 [SMILES: NS(=O)(=O)CCNC(=NS(=O)(=O)c1ccc(F)cc1)N1C[C@@H](c2ccccc2)C(c2ccc(F)cc2)=N1] |
| 54 | 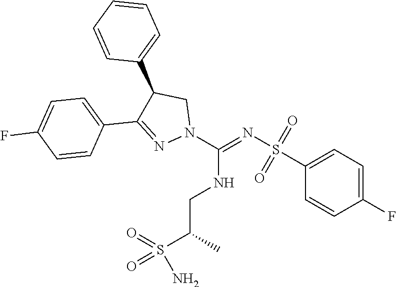 [SMILES: C[C@@H](CN/C(=N\S(=O)(=O)c1ccc(F)cc1)N1C[C@H](c2ccccc2)C(c2ccc(F)cc2)=N1)S(N)(=O)=O] |
| 55 | 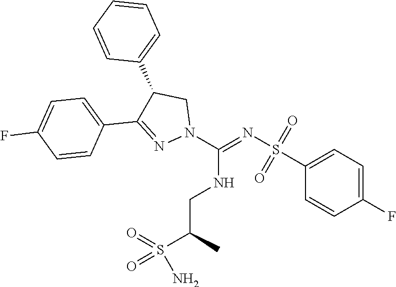 [SMILES: C[C@H](CN/C(=N\S(=O)(=O)c1ccc(F)cc1)N1C[C@@H](c2ccccc2)C(c2ccc(F)cc2)=N1)S(N)(=O)=O] |
| 56 | 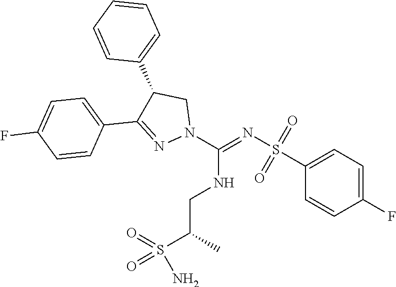 [SMILES: C[C@@H](CN/C(=N\S(=O)(=O)c1ccc(F)cc1)N1C[C@@H](c2ccccc2)C(c2ccc(F)cc2)=N1)S(N)(=O)=O] |
| 57 | 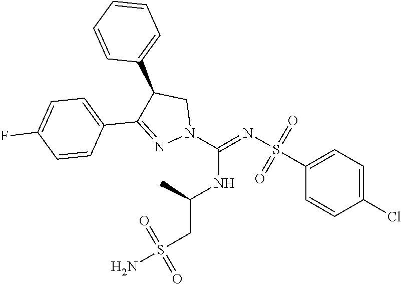 [SMILES: C[C@H](CS(N)(=O)=O)N/C(=N\S(=O)(=O)c1ccc(Cl)cc1)N1C[C@H](c2ccccc2)C(c2ccc(F)cc2)=N1] |
| 58 | 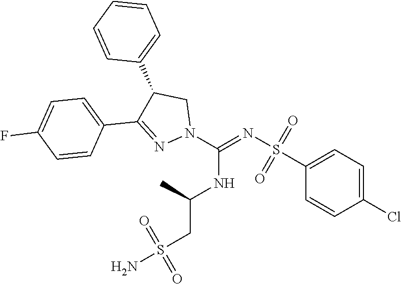 [SMILES: C[C@H](CS(N)(=O)=O)N/C(=N\S(=O)(=O)c1ccc(Cl)cc1)N1C[C@@H](c2ccccc2)C(c2ccc(F)cc2)=N1] |
| 59 | 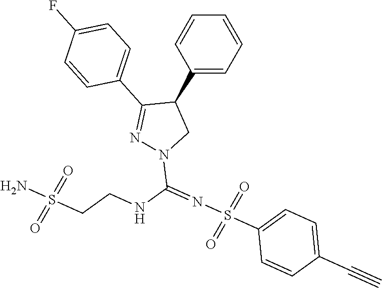 [SMILES: C#Cc1ccc(S(=O)(=O)N=C(NCCS(N)(=O)=O)N2C[C@H](c3ccccc3)C(c3ccc(F)cc3)=N2)cc1] |
| 60 | 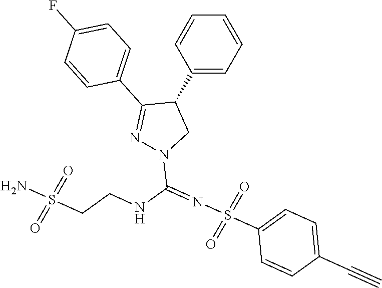 [SMILES: C#Cc1ccc(S(=O)(=O)N=C(NCCS(N)(=O)=O)N2C[C@@H](c3ccccc3)C(c3ccc(F)cc3)=N2)cc1] |
| 61 | 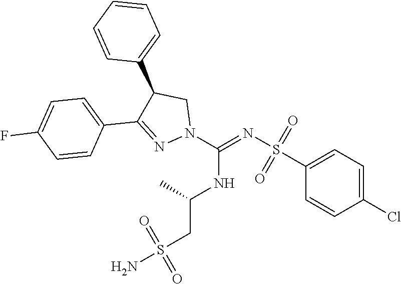 [SMILES: C[C@@H](CS(N)(=O)=O)N/C(=N\S(=O)(=O)c1ccc(Cl)cc1)N1C[C@H](c2ccccc2)C(c2ccc(F)cc2)=N1] |
| 62 | 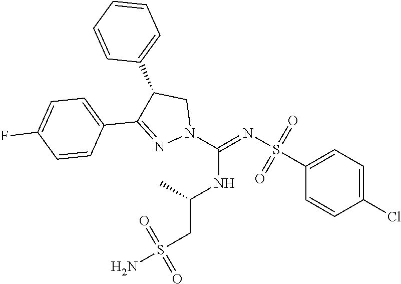 [SMILES: C[C@@H](CS(N)(=O)=O)N/C(=N\S(=O)(=O)c1ccc(Cl)cc1)N1C[C@@H](c2ccccc2)C(c2ccc(F)cc2)=N1] |
| 63 | 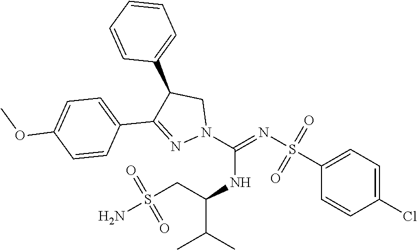 [SMILES: COc1ccc(C2=NN(/C(=N/S(=O)(=O)c3ccc(Cl)cc3)N[C@H](CS(N)(=O)=O)C(C)C)C[C@@H]2c2ccccc2)cc1] |
| 64 | 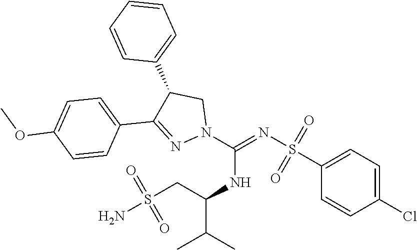 [SMILES: COc1ccc(C2=NN(/C(=N/S(=O)(=O)c3ccc(Cl)cc3)N[C@H](CS(N)(=O)=O)C(C)C)C[C@H]2c2ccccc2)cc1] |
| 65 | 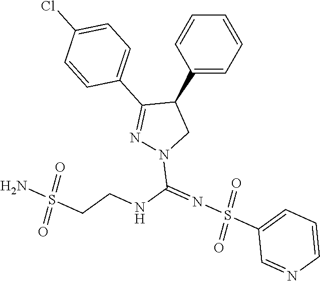 [SMILES: NS(=O)(=O)CCNC(=NS(=O)(=O)c1cccnc1)N1C[C@H](c2ccccc2)C(c2ccc(Cl)cc2)=N1] |
| 66 | 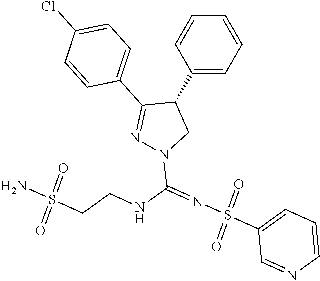 [SMILES: NS(=O)(=O)CCNC(=NS(=O)(=O)c1cccnc1)N1C[C@@H](c2ccccc2)C(c2ccc(Cl)cc2)=N1] |
| 67 | 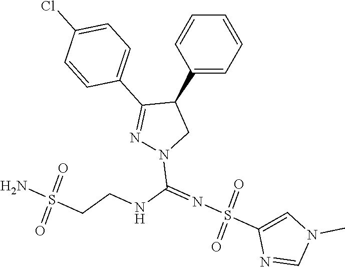 [SMILES: Cn1cnc(S(=O)(=O)N=C(NCCS(N)(=O)=O)N2C[C@H](c3ccccc3)C(c3ccc(Cl)cc3)=N2)c1] |
| 68 | 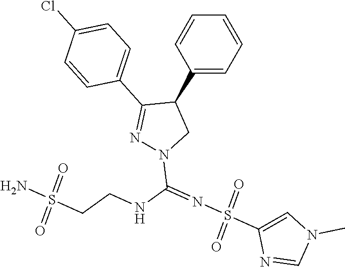 [SMILES: Cn1cnc(S(=O)(=O)N=C(NCCS(N)(=O)=O)N2C[C@H](c3ccccc3)C(c3ccc(Cl)cc3)=N2)c1] |
| 69 | 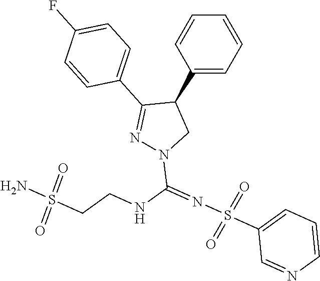 [SMILES: NS(=O)(=O)CCNC(=NS(=O)(=O)c1cccnc1)N1C[C@H](c2ccccc2)C(c2ccc(F)cc2)=N1] |
| 70 | 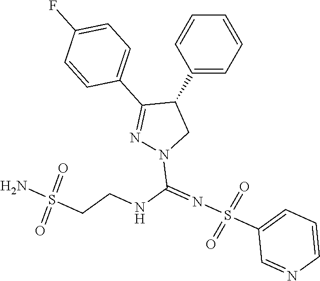 [SMILES: NS(=O)(=O)CCNC(=NS(=O)(=O)c1cccnc1)N1C[C@@H](c2ccccc2)C(c2ccc(F)cc2)=N1] |
| 71 | 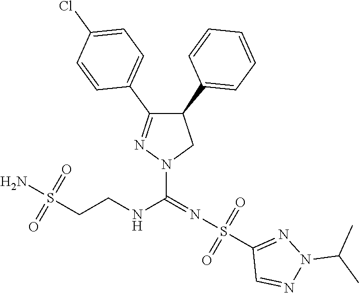 [SMILES: CC(C)n1ncc(S(=O)(=O)N=C(NCCS(N)(=O)=O)N2C[C@H](c3ccccc3)C(c3ccc(Cl)cc3)=N2)n1] |
| 72 | 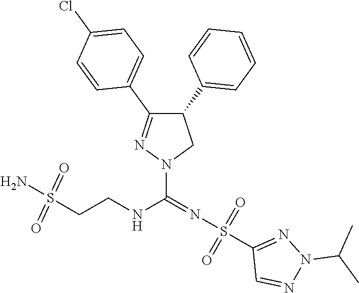 [SMILES: CC(C)n1ncc(S(=O)(=O)N=C(NCCS(N)(=O)=O)N2C[C@@H](c3ccccc3)C(c3ccc(Cl)cc3)=N2)n1] |
| 73 | 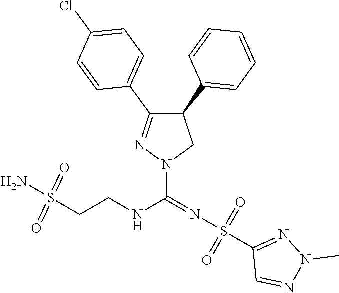 [SMILES: Cn1ncc(S(=O)(=O)N=C(NCCS(N)(=O)=O)N2C[C@H](c3ccccc3)C(c3ccc(Cl)cc3)=N2)n1] |
| 74 | 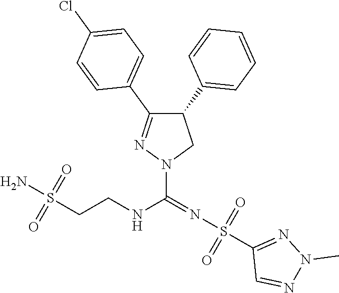 [SMILES: Cn1ncc(S(=O)(=O)N=C(NCCS(N)(=O)=O)N2C[C@@H](c3ccccc3)C(c3ccc(Cl)cc3)=N2)n1] |
| 75 | 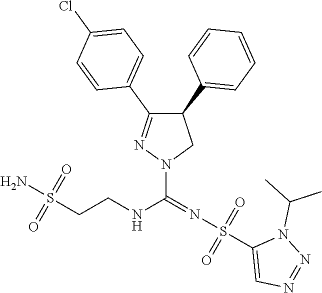 [SMILES: CC(C)n1nncc1S(=O)(=O)N=C(NCCS(N)(=O)=O)N1C[C@H](c2ccccc2)C(c2ccc(Cl)cc2)=N1] |
| 76 | 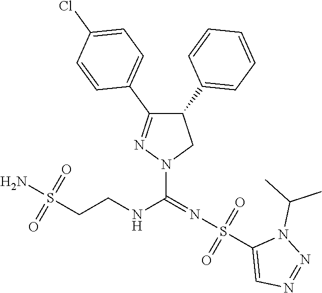 [SMILES: CC(C)n1nncc1S(=O)(=O)N=C(NCCS(N)(=O)=O)N1C[C@@H](c2ccccc2)C(c2ccc(Cl)cc2)=N1] |
| 77 | 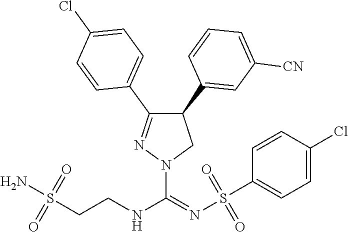 [SMILES: N#Cc1cccc([C@H]2CN(/C(=N\S(=O)(=O)c3ccc(Cl)cc3)NCCS(N)(=O)=O)N=C2c2ccc(Cl)cc2)c1] |
| 78 | 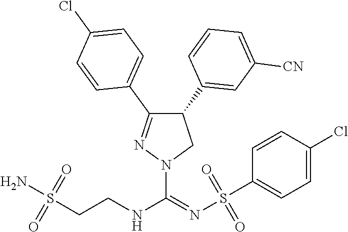 [SMILES: N#Cc1cccc([C@@H]2CN(/C(=N\S(=O)(=O)c3ccc(Cl)cc3)NCCS(N)(=O)=O)N=C2c2ccc(Cl)cc2)c1] |
| 81 | 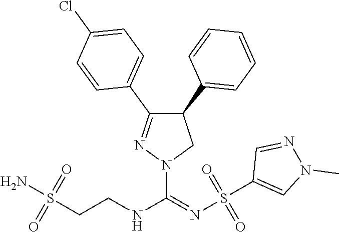 [SMILES: Cn1cc(S(=O)(=O)/N=C(/NCCS(N)(=O)=O)N2C[C@H](c3ccccc3)C(c3ccc(Cl)cc3)=N2)cn1] |
| 82 | 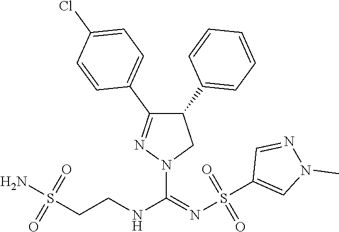 [SMILES: Cn1cc(S(=O)(=O)/N=C(/NCCS(N)(=O)=O)N2C[C@@H](c3ccccc3)C(c3ccc(Cl)cc3)=N2)cn1] |
| 83 | 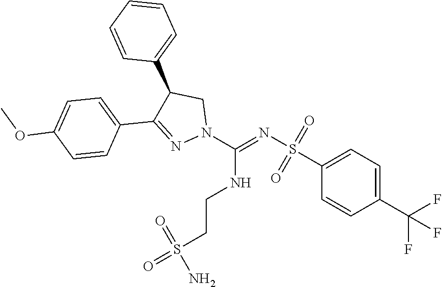 [SMILES: COc1ccc(C2=NN(/C(=N/S(=O)(=O)c3ccc(C(F)(F)F)cc3)NCCS(N)(=O)=O)C[C@@H]2c2ccccc2)cc1] |
| 84 | 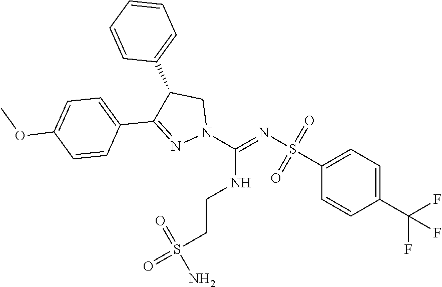 [SMILES: COc1ccc(C2=NN(/C(=N/S(=O)(=O)c3ccc(C(F)(F)F)cc3)NCCS(N)(=O)=O)C[C@H]2c2ccccc2)cc1] |
| 85 | 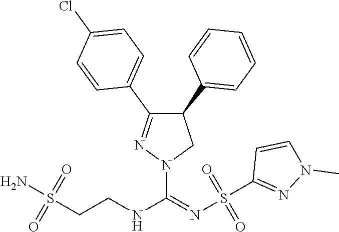 [SMILES: Cn1ccc(S(=O)(=O)/N=C(/NCCS(N)(=O)=O)N2C[C@H](c3ccccc3)C(c3ccc(Cl)cc3)=N2)n1] |
| 86 | 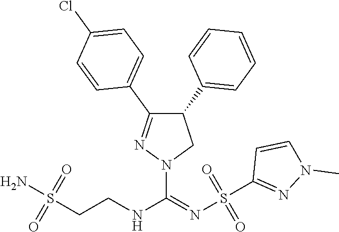 [SMILES: Cn1ccc(S(=O)(=O)/N=C(/NCCS(N)(=O)=O)N2C[C@@H](c3ccccc3)C(c3ccc(Cl)cc3)=N2)n1] |
| 87 | 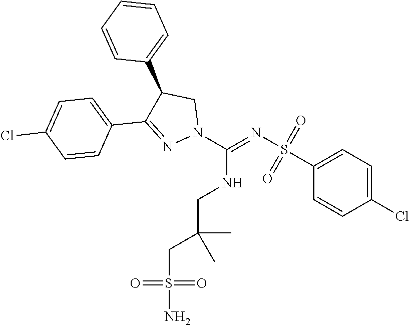 [SMILES: CC(C)(CN/C(=N\S(=O)(=O)c1ccc(Cl)cc1)N1C[C@H](c2ccccc2)C(c2ccc(Cl)cc2)=N1)CS(N)(=O)=O] |
| 88 | 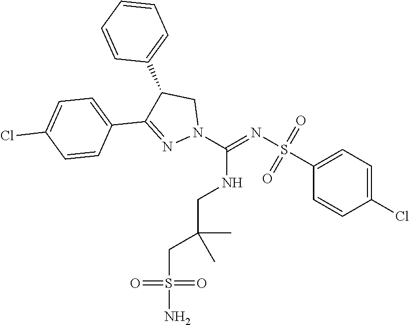 [SMILES: CC(C)(CN/C(=N\S(=O)(=O)c1ccc(Cl)cc1)N1C[C@@H](c2ccccc2)C(c2ccc(Cl)cc2)=N1)CS(N)(=O)=O] |
| 89 | 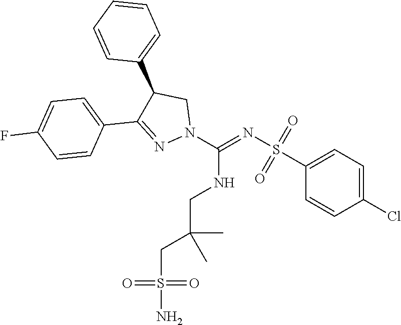 [SMILES: CC(C)(CN/C(=N\S(=O)(=O)c1ccc(Cl)cc1)N1C[C@H](c2ccccc2)C(c2ccc(F)cc2)=N1)CS(N)(=O)=O] |
| 90 | 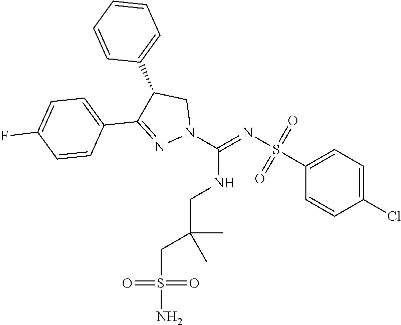 [SMILES: CC(C)(CN/C(=N\S(=O)(=O)c1ccc(Cl)cc1)N1C[C@@H](c2ccccc2)C(c2ccc(F)cc2)=N1)CS(N)(=O)=O] |
| 91 | 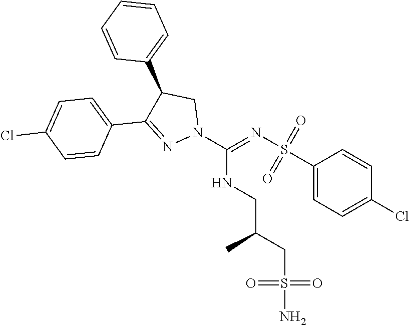 [SMILES: C[C@@H](CN/C(=N\S(=O)(=O)c1ccc(Cl)cc1)N1C[C@H](c2ccccc2)C(c2ccc(Cl)cc2)=N1)CS(N)(=O)=O] |
| 92 | 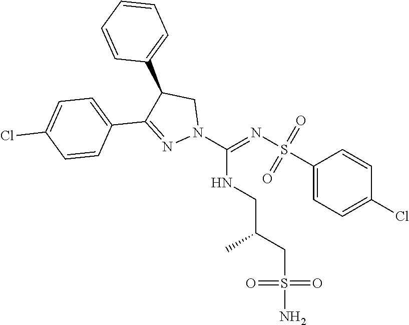 [SMILES: C[C@H](CN/C(=N\S(=O)(=O)c1ccc(Cl)cc1)N1C[C@H](c2ccccc2)C(c2ccc(Cl)cc2)=N1)CS(N)(=O)=O] |
| 93 | 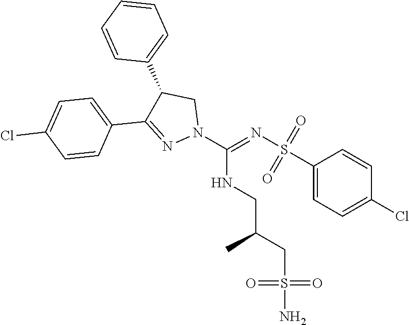 [SMILES: C[C@@H](CN/C(=N\S(=O)(=O)c1ccc(Cl)cc1)N1C[C@@H](c2ccccc2)C(c2ccc(Cl)cc2)=N1)CS(N)(=O)=O] |
| 94 | 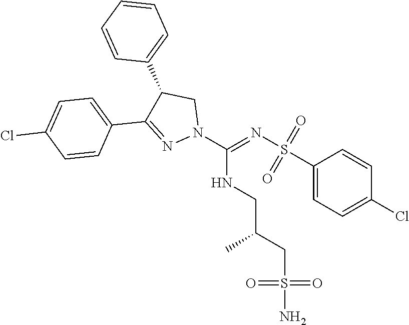 [SMILES: C[C@H](CN/C(=N\S(=O)(=O)c1ccc(Cl)cc1)N1C[C@@H](c2ccccc2)C(c2ccc(Cl)cc2)=N1)CS(N)(=O)=O] |
| 95 | 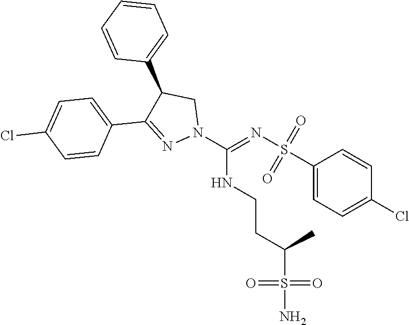 [SMILES: C[C@H](CCN/C(=N\S(=O)(=O)c1ccc(Cl)cc1)N1C[C@H](c2ccccc2)C(c2ccc(Cl)cc2)=N1)S(N)(=O)=O] |
| 96 | 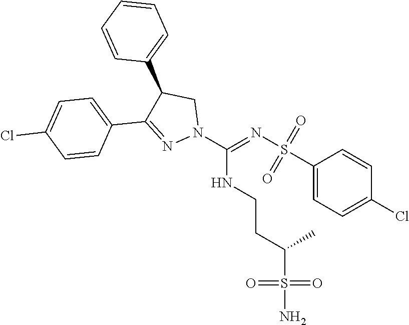 [SMILES: C[C@@H](CCN/C(=N\S(=O)(=O)c1ccc(Cl)cc1)N1C[C@H](c2ccccc2)C(c2ccc(Cl)cc2)=N1)S(N)(=O)=O] |
| 97 | 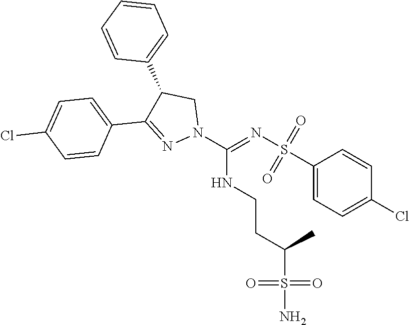 [SMILES: C[C@H](CCN/C(=N\S(=O)(=O)c1ccc(Cl)cc1)N1C[C@@H](c2ccccc2)C(c2ccc(Cl)cc2)=N1)S(N)(=O)=O] |
| 98 | 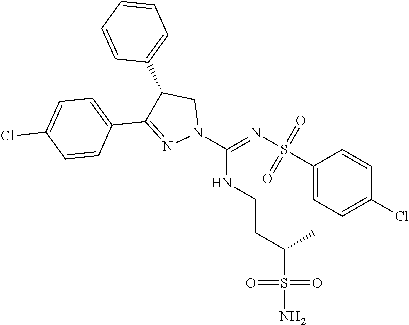 [SMILES: C[C@@H](CCN/C(=N\S(=O)(=O)c1ccc(Cl)cc1)N1C[C@@H](c2ccccc2)C(c2ccc(Cl)cc2)=N1)S(N)(=O)=O] |
| 99 | 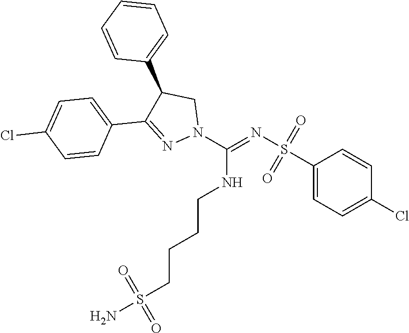 [SMILES: NS(=O)(=O)CCCCN/C(=N\S(=O)(=O)c1ccc(Cl)cc1)N1C[C@H](c2ccccc2)C(c2ccc(Cl)cc2)=N1] |
| 100 | 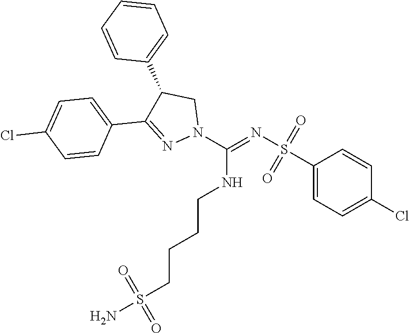 [SMILES: NS(=O)(=O)CCCCN/C(=N\S(=O)(=O)c1ccc(Cl)cc1)N1C[C@@H](c2ccccc2)C(c2ccc(Cl)cc2)=N1] |
| 101 | 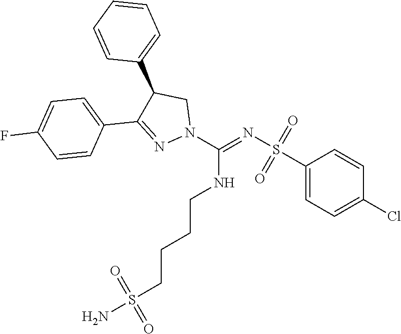 [SMILES: NS(=O)(=O)CCCCN/C(=N\S(=O)(=O)c1ccc(Cl)cc1)N1C[C@H](c2ccccc2)C(c2ccc(F)cc2)=N1] |
| 102 | 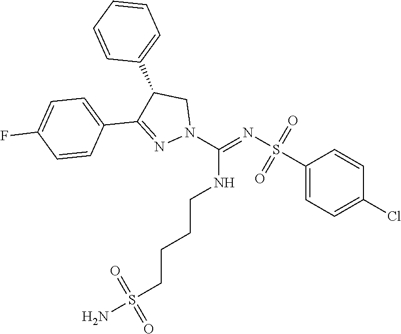 [SMILES: NS(=O)(=O)CCCCN/C(=N\S(=O)(=O)c1ccc(Cl)cc1)N1C[C@@H](c2ccccc2)C(c2ccc(F)cc2)=N1] |
| 103 | 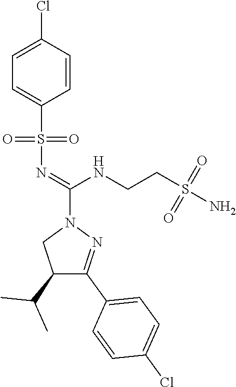 [SMILES: CC(C)[C@H]1CN(/C(=N/S(=O)(=O)c2ccc(Cl)cc2)NCCS(N)(=O)=O)N=C1c1ccc(Cl)cc1] |
| 104 | 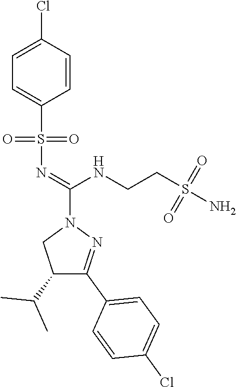 [SMILES: CC(C)[C@@H]1CN(/C(=N/S(=O)(=O)c2ccc(Cl)cc2)NCCS(N)(=O)=O)N=C1c1ccc(Cl)cc1] |
| 105 | 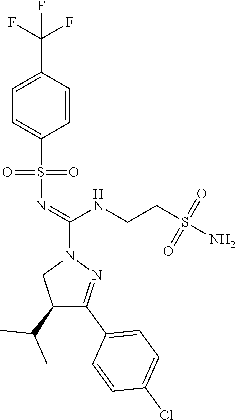 [SMILES: CC(C)[C@H]1CN(/C(=N/S(=O)(=O)c2ccc(C(F)(F)F)cc2)NCCS(N)(=O)=O)N=C1c1ccc(Cl)cc1] |
| 106 | 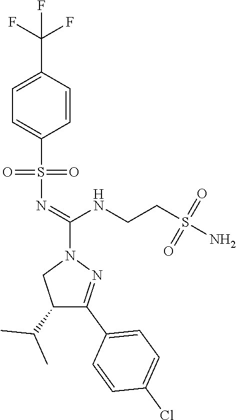 [SMILES: CC(C)[C@@H]1CN(/C(=N/S(=O)(=O)c2ccc(C(F)(F)F)cc2)NCCS(N)(=O)=O)N=C1c1ccc(Cl)cc1] |
| 107 | 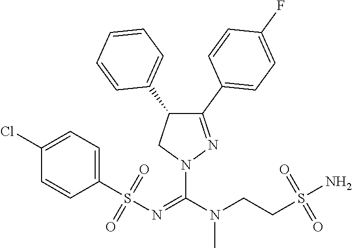 [SMILES: CN(CCS(N)(=O)=O)/C(=N/S(=O)(=O)c1ccc(Cl)cc1)N1C[C@H](c2ccccc2)C(c2ccc(F)cc2)=N1] |
| 108 | 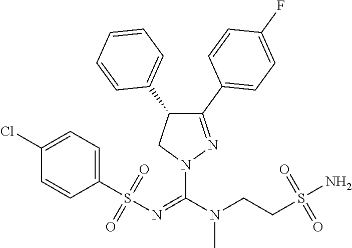 [SMILES: CN(CCS(N)(=O)=O)/C(=N/S(=O)(=O)c1ccc(Cl)cc1)N1C[C@H](c2ccccc2)C(c2ccc(F)cc2)=N1] |
| 109 | 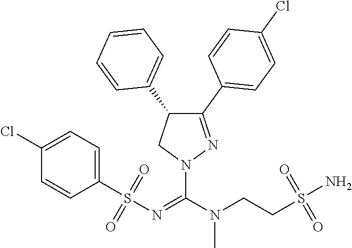 [SMILES: CN(CCS(N)(=O)=O)/C(=N/S(=O)(=O)c1ccc(Cl)cc1)N1C[C@H](c2ccccc2)C(c2ccc(Cl)cc2)=N1] |
| 110 | 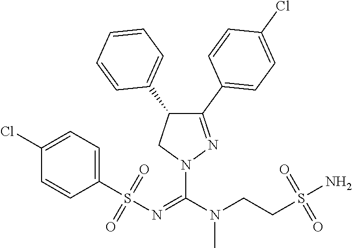 [SMILES: CN(CCS(N)(=O)=O)/C(=N/S(=O)(=O)c1ccc(Cl)cc1)N1C[C@H](c2ccccc2)C(c2ccc(Cl)cc2)=N1] |
| 111 | 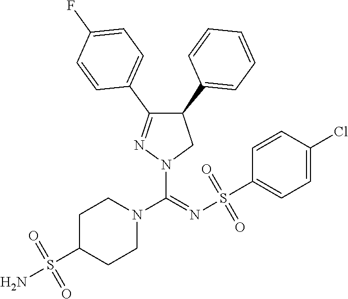 [SMILES: NS(=O)(=O)C1CCN(/C(=N/S(=O)(=O)c2ccc(Cl)cc2)N2C[C@H](c3ccccc3)C(c3ccc(F)cc3)=N2)CC1] |
| 112 | 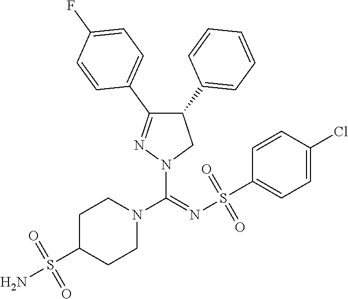 [SMILES: NS(=O)(=O)C1CCN(/C(=N/S(=O)(=O)c2ccc(Cl)cc2)N2C[C@@H](c3ccccc3)C(c3ccc(F)cc3)=N2)CC1] |
| 113 | 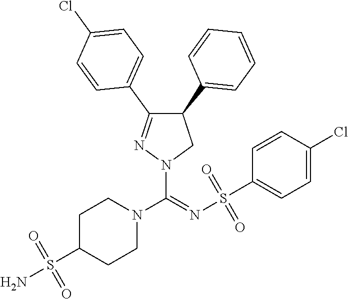 [SMILES: NS(=O)(=O)C1CCN(/C(=N/S(=O)(=O)c2ccc(Cl)cc2)N2C[C@H](c3ccccc3)C(c3ccc(Cl)cc3)=N2)CC1] |
| 114 | 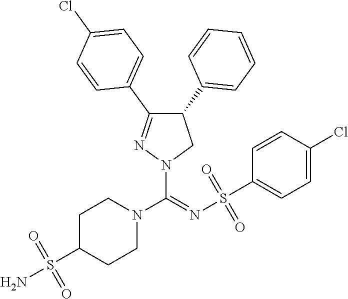 [SMILES: NS(=O)(=O)C1CCN(/C(=N/S(=O)(=O)c2ccc(Cl)cc2)N2C[C@@H](c3ccccc3)C(c3ccc(Cl)cc3)=N2)CC1] |
| 115 | 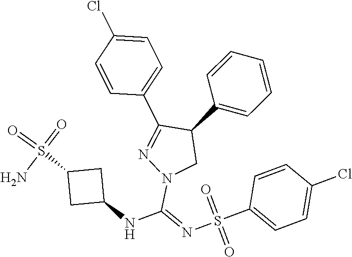 [SMILES: NS(=O)(=O)[C@H]1C[C@H](N/C(=N/S(=O)(=O)c2ccc(Cl)cc2)N2C[C@H](c3ccccc3)C(c3ccc(Cl)cc3)=N2)C1] |
| 116 | 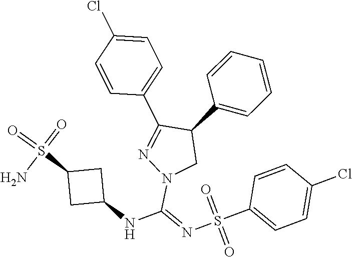 [SMILES: NS(=O)(=O)[C@H]1C[C@@H](N/C(=N/S(=O)(=O)c2ccc(Cl)cc2)N2C[C@H](c3ccccc3)C(c3ccc(Cl)cc3)=N2)C1] |
| 117 | 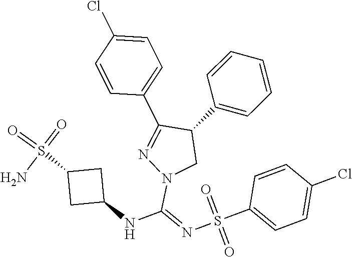 [SMILES: NS(=O)(=O)[C@H]1C[C@H](N/C(=N/S(=O)(=O)c2ccc(Cl)cc2)N2C[C@@H](c3ccccc3)C(c3ccc(Cl)cc3)=N2)C1] |
| 118 | 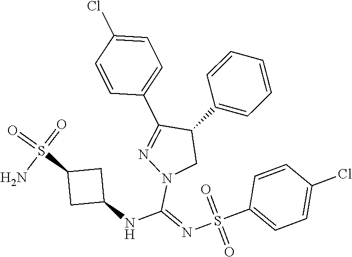 [SMILES: NS(=O)(=O)[C@H]1C[C@@H](N/C(=N/S(=O)(=O)c2ccc(Cl)cc2)N2C[C@@H](c3ccccc3)C(c3ccc(Cl)cc3)=N2)C1] |
| 119 | 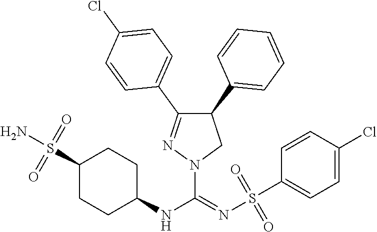 [SMILES: NS(=O)(=O)[C@H]1CC[C@@H](N/C(=N/S(=O)(=O)c2ccc(Cl)cc2)N2C[C@H](c3ccccc3)C(c3ccc(Cl)cc3)=N2)CC1] |
| 120 | 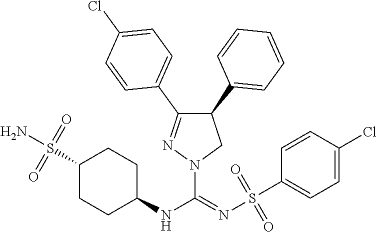 [SMILES: NS(=O)(=O)[C@H]1CC[C@H](N/C(=N/S(=O)(=O)c2ccc(Cl)cc2)N2C[C@H](c3ccccc3)C(c3ccc(Cl)cc3)=N2)CC1] |
| 121 | 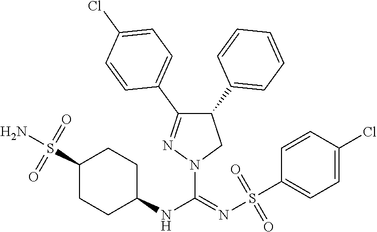 [SMILES: NS(=O)(=O)[C@H]1CC[C@@H](N/C(=N/S(=O)(=O)c2ccc(Cl)cc2)N2C[C@@H](c3ccccc3)C(c3ccc(Cl)cc3)=N2)CC1] |
| 122 | 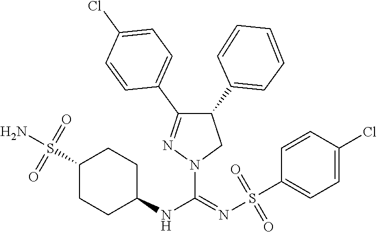 [SMILES: NS(=O)(=O)[C@H]1CC[C@H](N/C(=N/S(=O)(=O)c2ccc(Cl)cc2)N2C[C@@H](c3ccccc3)C(c3ccc(Cl)cc3)=N2)CC1] |
| 123 | 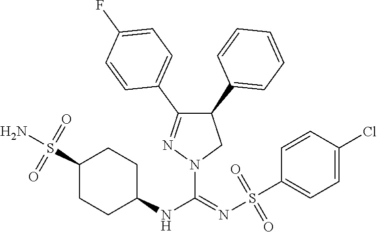 [SMILES: NS(=O)(=O)[C@H]1CC[C@@H](N/C(=N/S(=O)(=O)c2ccc(Cl)cc2)N2C[C@H](c3ccccc3)C(c3ccc(F)cc3)=N2)CC1] |
| 124 | 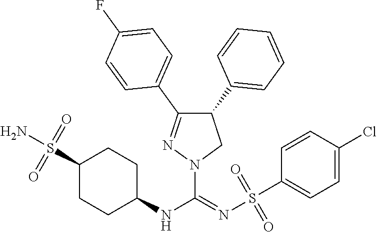 [SMILES: NS(=O)(=O)[C@H]1CC[C@@H](N/C(=N/S(=O)(=O)c2ccc(Cl)cc2)N2C[C@@H](c3ccccc3)C(c3ccc(F)cc3)=N2)CC1] |
| 125 | 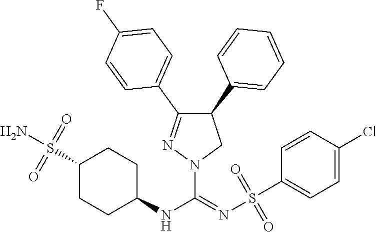 [SMILES: NS(=O)(=O)[C@H]1CC[C@H](N/C(=N/S(=O)(=O)c2ccc(Cl)cc2)N2C[C@H](c3ccccc3)C(c3ccc(F)cc3)=N2)CC1] |
| 126 | 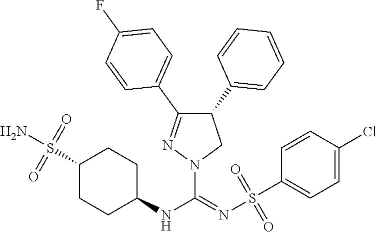 [SMILES: NS(=O)(=O)[C@H]1CC[C@H](N/C(=N/S(=O)(=O)c2ccc(Cl)cc2)N2C[C@@H](c3ccccc3)C(c3ccc(F)cc3)=N2)CC1] |
| 127 | 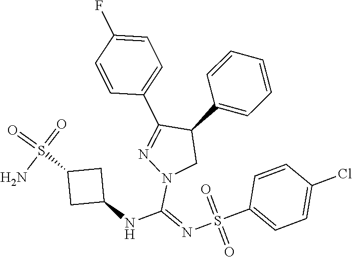 [SMILES: NS(=O)(=O)[C@H]1C[C@H](N/C(=N/S(=O)(=O)c2ccc(Cl)cc2)N2C[C@H](c3ccccc3)C(c3ccc(F)cc3)=N2)C1] |
| 128 | 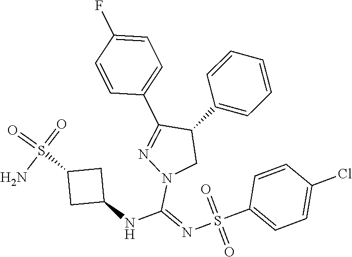 [SMILES: NS(=O)(=O)[C@H]1C[C@H](N/C(=N/S(=O)(=O)c2ccc(Cl)cc2)N2C[C@@H](c3ccccc3)C(c3ccc(F)cc3)=N2)C1] |
| 129 | 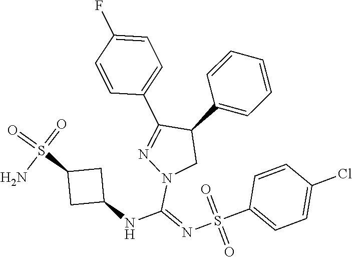 [SMILES: NS(=O)(=O)[C@H]1C[C@@H](N/C(=N/S(=O)(=O)c2ccc(Cl)cc2)N2C[C@H](c3ccccc3)C(c3ccc(F)cc3)=N2)C1] |
| 130 | 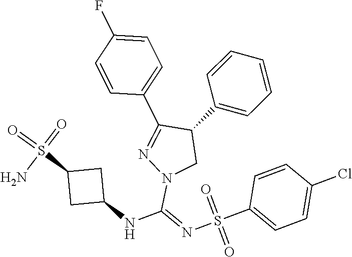 [SMILES: NS(=O)(=O)[C@H]1C[C@@H](N/C(=N/S(=O)(=O)c2ccc(Cl)cc2)N2C[C@@H](c3ccccc3)C(c3ccc(F)cc3)=N2)C1] |
| 131 | 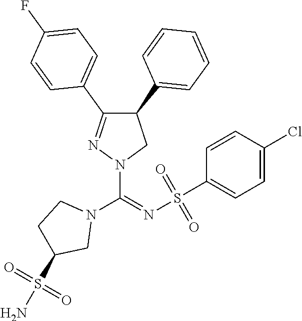 [SMILES: NS(=O)(=O)[C@H]1CCN(/C(=N/S(=O)(=O)c2ccc(Cl)cc2)N2C[C@H](c3ccccc3)C(c3ccc(F)cc3)=N2)C1] |
| 132 | 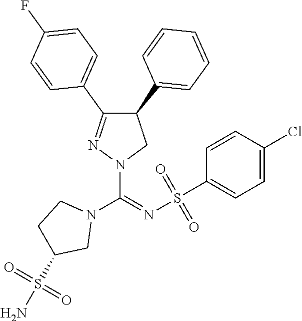 [SMILES: NS(=O)(=O)[C@@H]1CCN(/C(=N/S(=O)(=O)c2ccc(Cl)cc2)N2C[C@H](c3ccccc3)C(c3ccc(F)cc3)=N2)C1] |
| 133 | 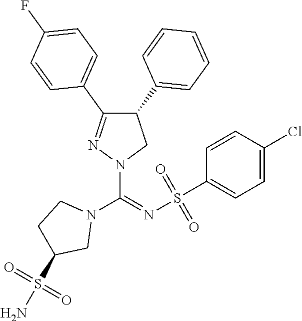 [SMILES: NS(=O)(=O)[C@H]1CCN(/C(=N/S(=O)(=O)c2ccc(Cl)cc2)N2C[C@@H](c3ccccc3)C(c3ccc(F)cc3)=N2)C1] |
| 134 | 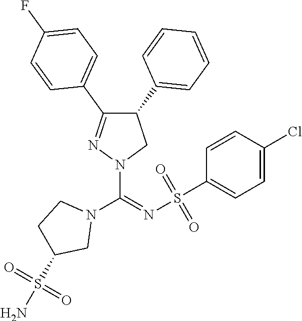 [SMILES: NS(=O)(=O)[C@@H]1CCN(/C(=N/S(=O)(=O)c2ccc(Cl)cc2)N2C[C@@H](c3ccccc3)C(c3ccc(F)cc3)=N2)C1] |
| 135 | 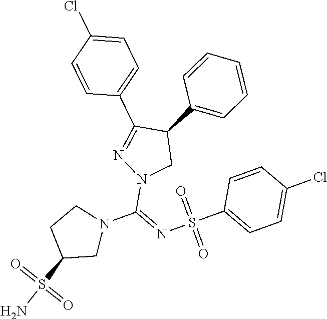 [SMILES: NS(=O)(=O)[C@H]1CCN(/C(=N/S(=O)(=O)c2ccc(Cl)cc2)N2C[C@H](c3ccccc3)C(c3ccc(Cl)cc3)=N2)C1] |
| 136 | 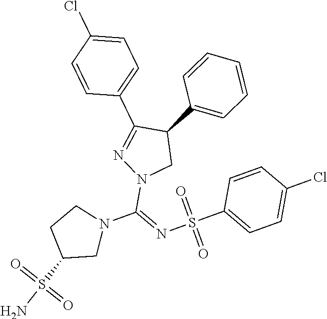 [SMILES: NS(=O)(=O)[C@@H]1CCN(/C(=N/S(=O)(=O)c2ccc(Cl)cc2)N2C[C@H](c3ccccc3)C(c3ccc(Cl)cc3)=N2)C1] |
| 137 | 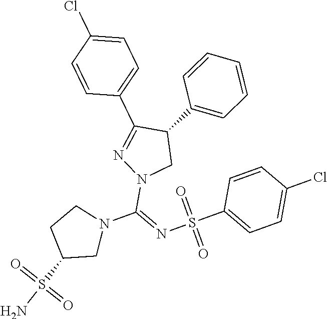 [SMILES: NS(=O)(=O)[C@@H]1CCN(/C(=N/S(=O)(=O)c2ccc(Cl)cc2)N2C[C@@H](c3ccccc3)C(c3ccc(Cl)cc3)=N2)C1] |
| 138 | 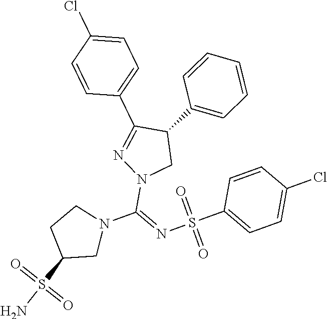 [SMILES: NS(=O)(=O)[C@H]1CCN(/C(=N/S(=O)(=O)c2ccc(Cl)cc2)N2C[C@@H](c3ccccc3)C(c3ccc(Cl)cc3)=N2)C1] |
| 139 | 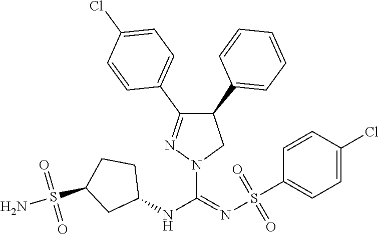 [SMILES: NS(=O)(=O)[C@H]1CC[C@H](N/C(=N/S(=O)(=O)c2ccc(Cl)cc2)N2C[C@H](c3ccccc3)C(c3ccc(Cl)cc3)=N2)C1] |
| 140 | 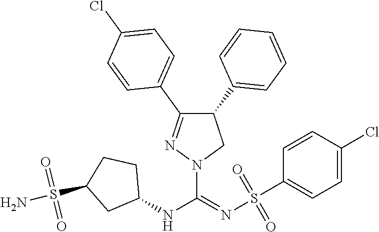 [SMILES: NS(=O)(=O)[C@H]1CC[C@H](N/C(=N/S(=O)(=O)c2ccc(Cl)cc2)N2C[C@@H](c3ccccc3)C(c3ccc(Cl)cc3)=N2)C1] |
| 141 | 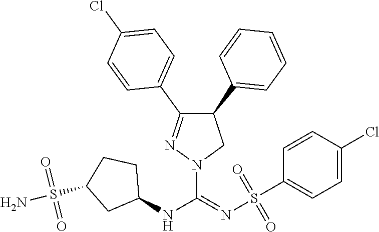 [SMILES: NS(=O)(=O)[C@@H]1CC[C@@H](N/C(=N/S(=O)(=O)c2ccc(Cl)cc2)N2C[C@H](c3ccccc3)C(c3ccc(Cl)cc3)=N2)C1] |
| 142 | 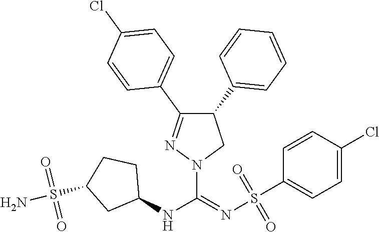 [SMILES: NS(=O)(=O)[C@@H]1CC[C@@H](N/C(=N/S(=O)(=O)c2ccc(Cl)cc2)N2C[C@@H](c3ccccc3)C(c3ccc(Cl)cc3)=N2)C1] |
| 143 | 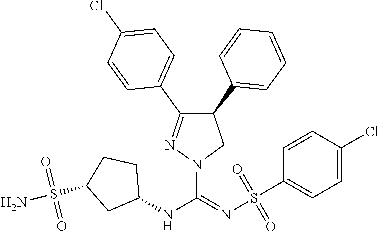 [SMILES: NS(=O)(=O)[C@@H]1CC[C@H](N/C(=N/S(=O)(=O)c2ccc(Cl)cc2)N2C[C@H](c3ccccc3)C(c3ccc(Cl)cc3)=N2)C1] |
| 144 | 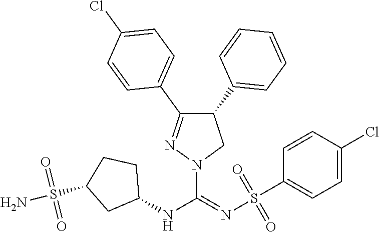 [SMILES: NS(=O)(=O)[C@@H]1CC[C@H](N/C(=N/S(=O)(=O)c2ccc(Cl)cc2)N2C[C@@H](c3ccccc3)C(c3ccc(Cl)cc3)=N2)C1] |
| 145 | 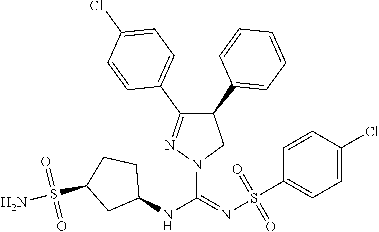 [SMILES: NS(=O)(=O)[C@H]1CC[C@@H](N/C(=N/S(=O)(=O)c2ccc(Cl)cc2)N2C[C@H](c3ccccc3)C(c3ccc(Cl)cc3)=N2)C1] |
| 146 | 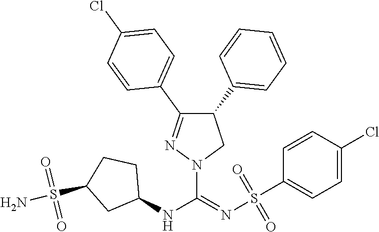 [SMILES: NS(=O)(=O)[C@H]1CC[C@@H](N/C(=N/S(=O)(=O)c2ccc(Cl)cc2)N2C[C@@H](c3ccccc3)C(c3ccc(Cl)cc3)=N2)C1] |
| 147 | 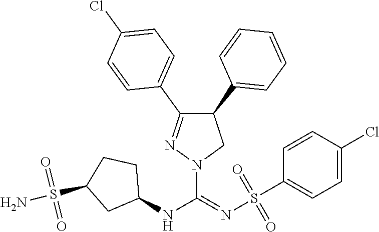 [SMILES: NS(=O)(=O)[C@H]1CC[C@@H](N/C(=N/S(=O)(=O)c2ccc(Cl)cc2)N2C[C@H](c3ccccc3)C(c3ccc(Cl)cc3)=N2)C1] |
| 148 | 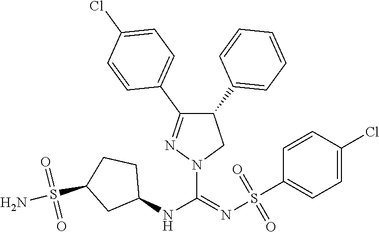 [SMILES: NS(=O)(=O)[C@H]1CC[C@@H](N/C(=N/S(=O)(=O)c2ccc(Cl)cc2)N2C[C@@H](c3ccccc3)C(c3ccc(Cl)cc3)=N2)C1] |
| 149 | 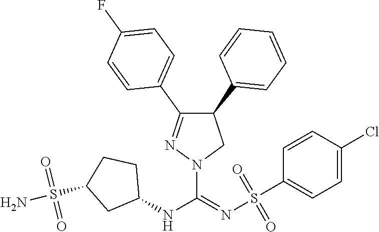 [SMILES: NS(=O)(=O)[C@@H]1CC[C@H](N/C(=N/S(=O)(=O)c2ccc(Cl)cc2)N2C[C@H](c3ccccc3)C(c3ccc(F)cc3)=N2)C1] |
| 150 | 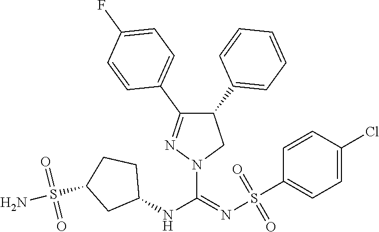 [SMILES: NS(=O)(=O)[C@@H]1CC[C@H](N/C(=N/S(=O)(=O)c2ccc(Cl)cc2)N2C[C@@H](c3ccccc3)C(c3ccc(F)cc3)=N2)C1] |
| 151 | 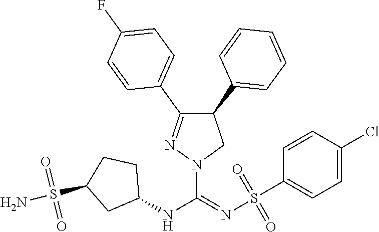 [SMILES: NS(=O)(=O)[C@H]1CC[C@H](N/C(=N/S(=O)(=O)c2ccc(Cl)cc2)N2C[C@H](c3ccccc3)C(c3ccc(F)cc3)=N2)C1] |
| 152 | 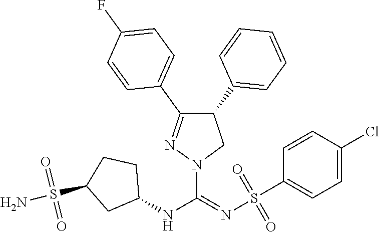 [SMILES: NS(=O)(=O)[C@H]1CC[C@H](N/C(=N/S(=O)(=O)c2ccc(Cl)cc2)N2C[C@@H](c3ccccc3)C(c3ccc(F)cc3)=N2)C1] |
| 153 | 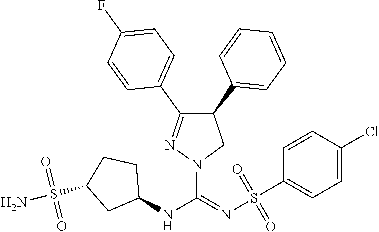 [SMILES: NS(=O)(=O)[C@@H]1CC[C@@H](N/C(=N/S(=O)(=O)c2ccc(Cl)cc2)N2C[C@H](c3ccccc3)C(c3ccc(F)cc3)=N2)C1] |
| 154 | 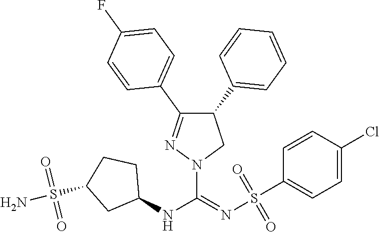 [SMILES: NS(=O)(=O)[C@@H]1CC[C@@H](N/C(=N/S(=O)(=O)c2ccc(Cl)cc2)N2C[C@@H](c3ccccc3)C(c3ccc(F)cc3)=N2)C1] |
| 155 | 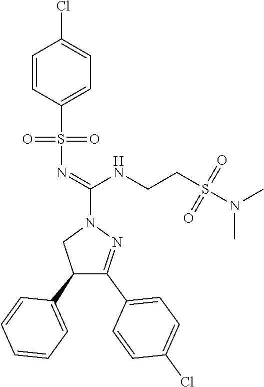 [SMILES: CN(C)S(=O)(=O)CCN/C(=N\S(=O)(=O)c1ccc(Cl)cc1)N1C[C@H](c2ccccc2)C(c2ccc(Cl)cc2)=N1] |
| 156 | 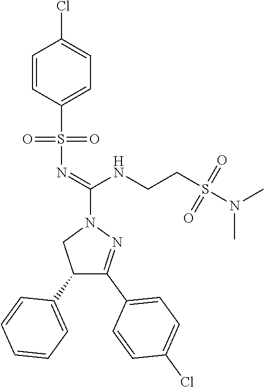 [SMILES: CN(C)S(=O)(=O)CCN/C(=N\S(=O)(=O)c1ccc(Cl)cc1)N1C[C@@H](c2ccccc2)C(c2ccc(Cl)cc2)=N1] |
| 157 | 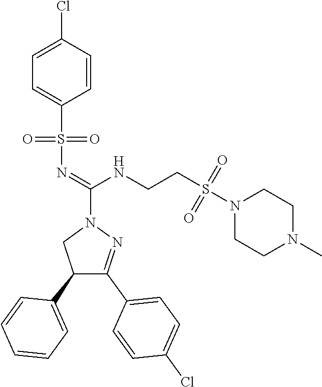 [SMILES: CN1CCN(S(=O)(=O)CCN/C(=N\S(=O)(=O)c2ccc(Cl)cc2)N2C[C@H](c3ccccc3)C(c3ccc(Cl)cc3)=N2)CC1] |
| 158 | 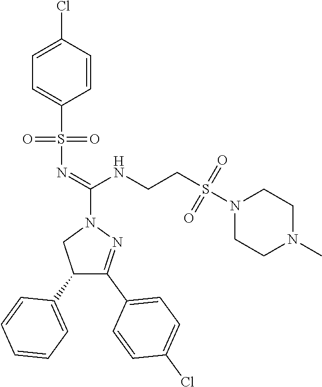 [SMILES: CN1CCN(S(=O)(=O)CCN/C(=N\S(=O)(=O)c2ccc(Cl)cc2)N2C[C@@H](c3ccccc3)C(c3ccc(Cl)cc3)=N2)CC1] |
| 159 | 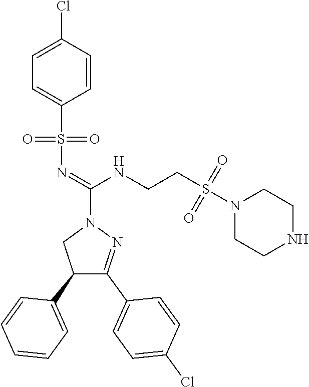 [SMILES: O=S(=O)(/N=C(\NCCS(=O)(=O)N1CCNCC1)N1C[C@H](c2ccccc2)C(c2ccc(Cl)cc2)=N1)c1ccc(Cl)cc1] |
| 160 | 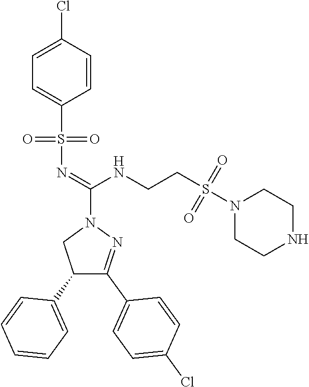 [SMILES: O=S(=O)(/N=C(\NCCS(=O)(=O)N1CCNCC1)N1C[C@@H](c2ccccc2)C(c2ccc(Cl)cc2)=N1)c1ccc(Cl)cc1] |
| 161 | 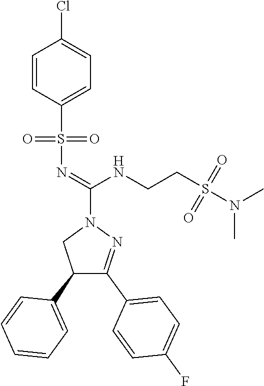 [SMILES: CN(C)S(=O)(=O)CCN/C(=N\S(=O)(=O)c1ccc(Cl)cc1)N1C[C@H](c2ccccc2)C(c2ccc(F)cc2)=N1] |
| 162 | 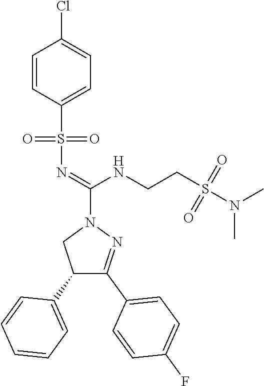 [SMILES: CN(C)S(=O)(=O)CCN/C(=N\S(=O)(=O)c1ccc(Cl)cc1)N1C[C@@H](c2ccccc2)C(c2ccc(F)cc2)=N1] |
| 163 | 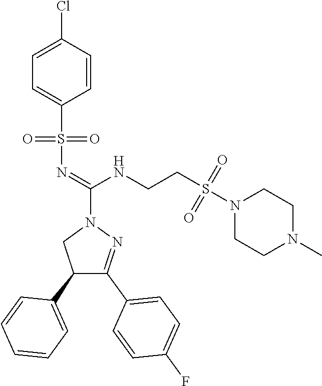 [SMILES: CN1CCN(S(=O)(=O)CCN/C(=N\S(=O)(=O)c2ccc(Cl)cc2)N2C[C@H](c3ccccc3)C(c3ccc(F)cc3)=N2)CC1] |
| 164 | 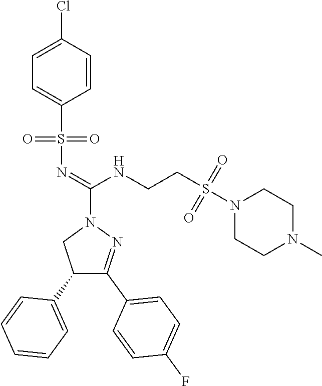 [SMILES: CN1CCN(S(=O)(=O)CCN/C(=N\S(=O)(=O)c2ccc(Cl)cc2)N2C[C@@H](c3ccccc3)C(c3ccc(F)cc3)=N2)CC1] |
| 165 | 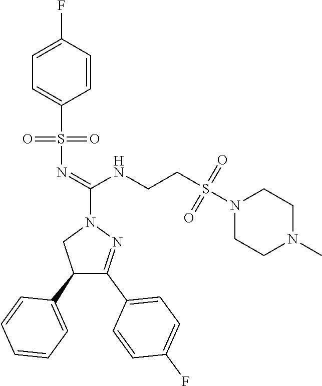 [SMILES: CN1CCN(S(=O)(=O)CCN/C(=N\S(=O)(=O)c2ccc(F)cc2)N2C[C@H](c3ccccc3)C(c3ccc(F)cc3)=N2)CC1] |
| 166 | 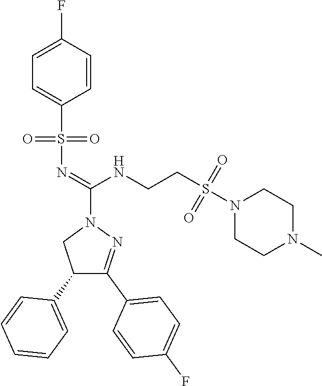 [SMILES: CN1CCN(S(=O)(=O)CCN/C(=N\S(=O)(=O)c2ccc(F)cc2)N2C[C@@H](c3ccccc3)C(c3ccc(F)cc3)=N2)CC1] |
| 167 | 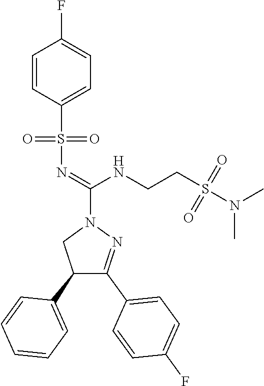 [SMILES: CN(C)S(=O)(=O)CCN/C(=N\S(=O)(=O)c1ccc(F)cc1)N1C[C@H](c2ccccc2)C(c2ccc(F)cc2)=N1] |
| 168 | 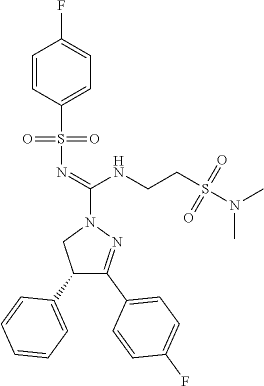 [SMILES: CN(C)S(=O)(=O)CCN/C(=N\S(=O)(=O)c1ccc(F)cc1)N1C[C@@H](c2ccccc2)C(c2ccc(F)cc2)=N1] |
| 169 | 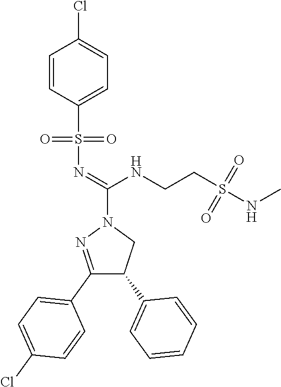 [SMILES: CNS(=O)(=O)CCN/C(=N\S(=O)(=O)c1ccc(Cl)cc1)N1C[C@H](c2ccccc2)C(c2ccc(Cl)cc2)=N1] |
| 170 | 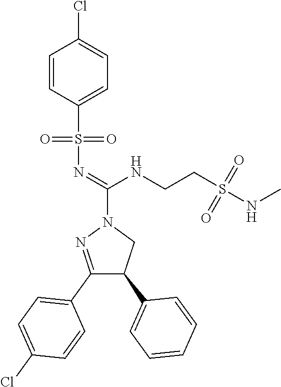 [SMILES: CNS(=O)(=O)CCN/C(=N\S(=O)(=O)c1ccc(Cl)cc1)N1C[C@@H](c2ccccc2)C(c2ccc(Cl)cc2)=N1] |
| 171 | 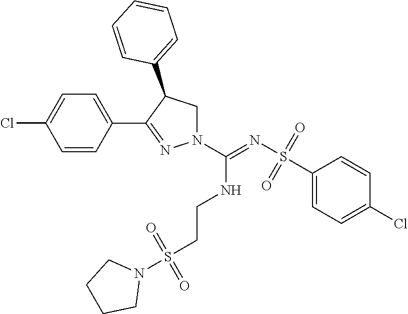 [SMILES: O=S(=O)(/N=C(\NCCS(=O)(=O)N1CCCC1)N1C[C@H](c2ccccc2)C(c2ccc(Cl)cc2)=N1)c1ccc(Cl)cc1] |
| 172 | 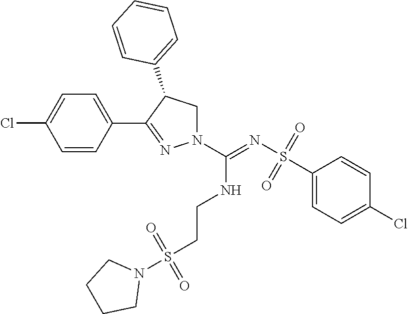 [SMILES: O=S(=O)(/N=C(\NCCS(=O)(=O)N1CCCC1)N1C[C@@H](c2ccccc2)C(c2ccc(Cl)cc2)=N1)c1ccc(Cl)cc1] |
| 173 | 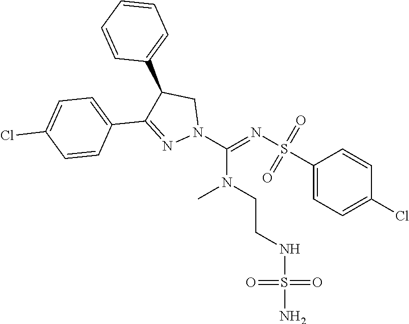 [SMILES: CN(CCNS(N)(=O)=O)/C(=N\S(=O)(=O)c1ccc(Cl)cc1)N1C[C@H](c2ccccc2)C(c2ccc(Cl)cc2)=N1] |
| 174 | 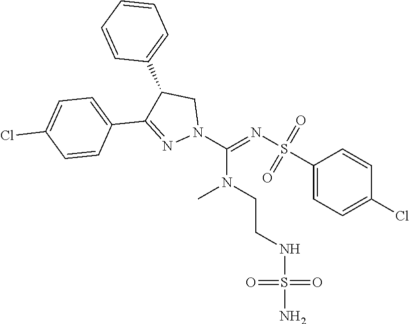 [SMILES: CN(CCNS(N)(=O)=O)/C(=N\S(=O)(=O)c1ccc(Cl)cc1)N1C[C@@H](c2ccccc2)C(c2ccc(Cl)cc2)=N1] |
| 175 | 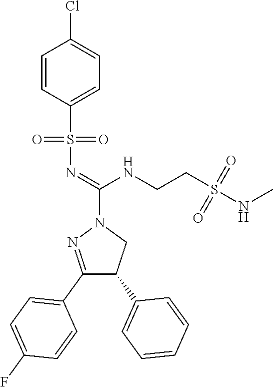 [SMILES: CNS(=O)(=O)CCN/C(=N\S(=O)(=O)c1ccc(Cl)cc1)N1C[C@H](c2ccccc2)C(c2ccc(F)cc2)=N1] |
| 176 | 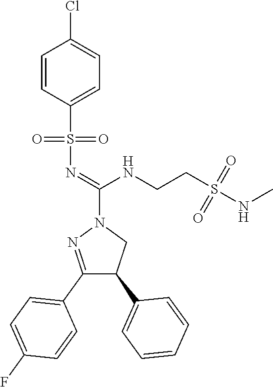 [SMILES: CNS(=O)(=O)CCN/C(=N\S(=O)(=O)c1ccc(Cl)cc1)N1C[C@@H](c2ccccc2)C(c2ccc(F)cc2)=N1] |
| 177 | 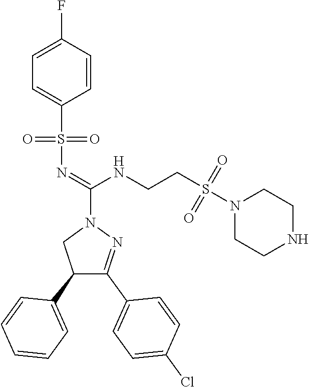 [SMILES: O=S(=O)(/N=C(\NCCS(=O)(=O)N1CCNCC1)N1C[C@H](c2ccccc2)C(c2ccc(Cl)cc2)=N1)c1ccc(F)cc1] |
| 178 | 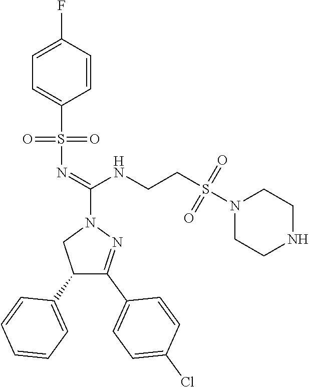 [SMILES: O=S(=O)(/N=C(\NCCS(=O)(=O)N1CCNCC1)N1C[C@@H](c2ccccc2)C(c2ccc(Cl)cc2)=N1)c1ccc(F)cc1] |
| 179 | 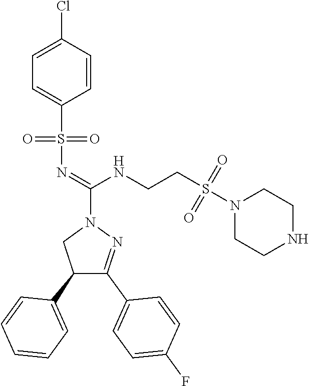 [SMILES: O=S(=O)(/N=C(\NCCS(=O)(=O)N1CCNCC1)N1C[C@H](c2ccccc2)C(c2ccc(F)cc2)=N1)c1ccc(Cl)cc1] |
| 180 | 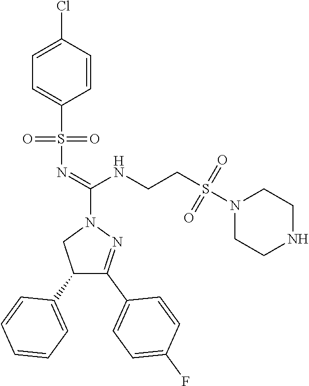 [SMILES: O=S(=O)(/N=C(\NCCS(=O)(=O)N1CCNCC1)N1C[C@@H](c2ccccc2)C(c2ccc(F)cc2)=N1)c1ccc(Cl)cc1] |
| 181 | 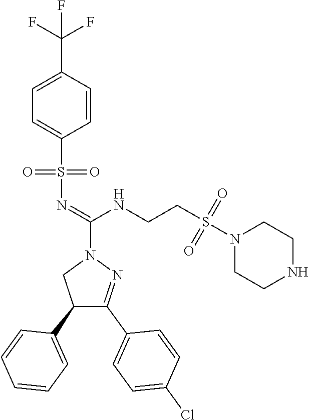 [SMILES: O=S(=O)(/N=C(\NCCS(=O)(=O)N1CCNCC1)N1C[C@H](c2ccccc2)C(c2ccc(Cl)cc2)=N1)c1ccc(C(F)(F)F)cc1] |
| 182 | 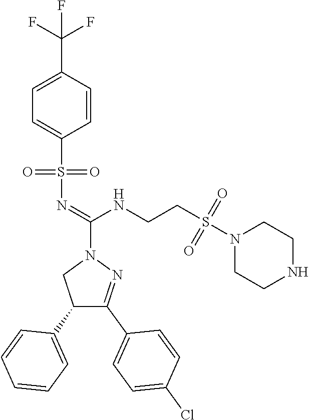 [SMILES: O=S(=O)(/N=C(\NCCS(=O)(=O)N1CCNCC1)N1C[C@@H](c2ccccc2)C(c2ccc(Cl)cc2)=N1)c1ccc(C(F)(F)F)cc1] |
| 183 | 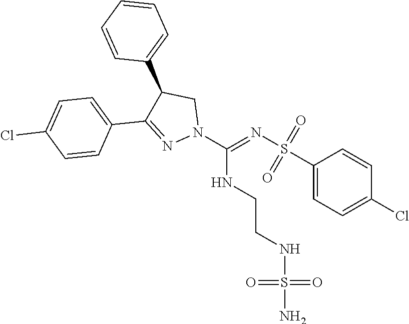 [SMILES: NS(=O)(=O)NCCN/C(=N\S(=O)(=O)c1ccc(Cl)cc1)N1C[C@H](c2ccccc2)C(c2ccc(Cl)cc2)=N1] |
| 184 | 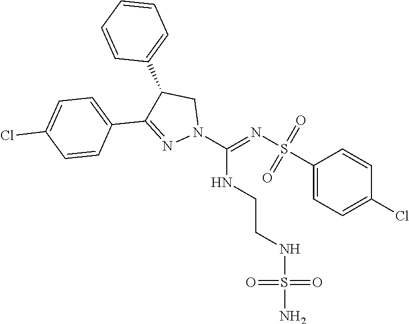 [SMILES: NS(=O)(=O)NCCN/C(=N\S(=O)(=O)c1ccc(Cl)cc1)N1C[C@@H](c2ccccc2)C(c2ccc(Cl)cc2)=N1] |
| 185 | 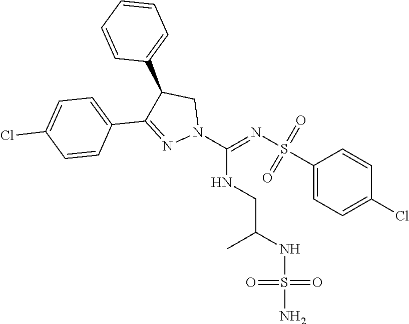 [SMILES: CC(CN/C(=N\S(=O)(=O)c1ccc(Cl)cc1)N1C[C@H](c2ccccc2)C(c2ccc(Cl)cc2)=N1)NS(N)(=O)=O] |
| 186 | 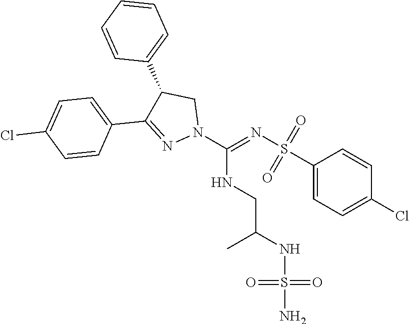 [SMILES: CC(CN/C(=N\S(=O)(=O)c1ccc(Cl)cc1)N1C[C@@H](c2ccccc2)C(c2ccc(Cl)cc2)=N1)NS(N)(=O)=O] |
| 187 | 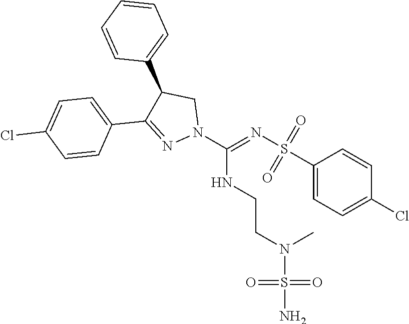 [SMILES: CN(CCN/C(=N\S(=O)(=O)c1ccc(Cl)cc1)N1C[C@H](c2ccccc2)C(c2ccc(Cl)cc2)=N1)S(N)(=O)=O] |
| 188 | 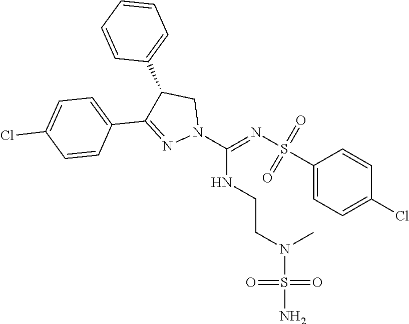 [SMILES: CN(CCN/C(=N\S(=O)(=O)c1ccc(Cl)cc1)N1C[C@@H](c2ccccc2)C(c2ccc(Cl)cc2)=N1)S(N)(=O)=O] |
| 189 | 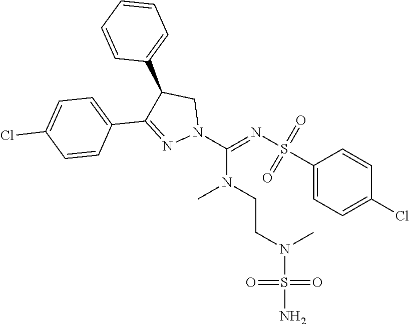 [SMILES: CN(CCN(C)S(N)(=O)=O)/C(=N\S(=O)(=O)c1ccc(Cl)cc1)N1C[C@H](c2ccccc2)C(c2ccc(Cl)cc2)=N1] |
| 190 | 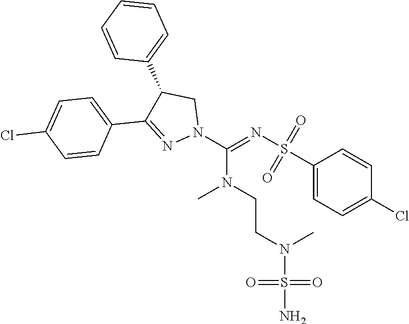 [SMILES: CN(CCN(C)S(N)(=O)=O)/C(=N\S(=O)(=O)c1ccc(Cl)cc1)N1C[C@@H](c2ccccc2)C(c2ccc(Cl)cc2)=N1] |
| 191 | 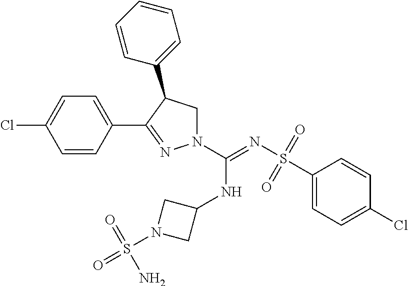 [SMILES: NS(=O)(=O)N1CC(N/C(=N\S(=O)(=O)c2ccc(Cl)cc2)N2C[C@H](c3ccccc3)C(c3ccc(Cl)cc3)=N2)C1] |
| 192 | 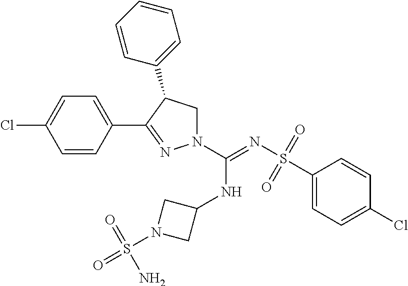 [SMILES: NS(=O)(=O)N1CC(N/C(=N\S(=O)(=O)c2ccc(Cl)cc2)N2C[C@@H](c3ccccc3)C(c3ccc(Cl)cc3)=N2)C1] |
| 193 | 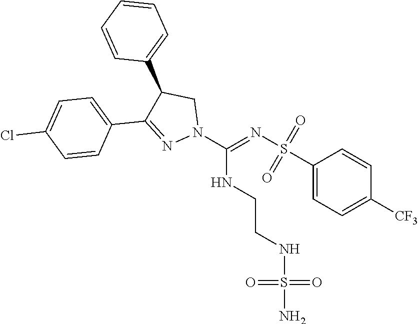 [SMILES: NS(=O)(=O)NCCN/C(=N\S(=O)(=O)c1ccc(C(F)(F)F)cc1)N1C[C@H](c2ccccc2)C(c2ccc(Cl)cc2)=N1] |
| 194 | 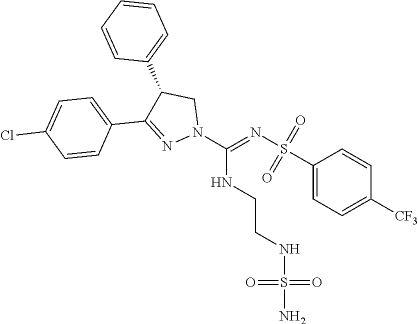 [SMILES: NS(=O)(=O)NCCN/C(=N\S(=O)(=O)c1ccc(C(F)(F)F)cc1)N1C[C@@H](c2ccccc2)C(c2ccc(Cl)cc2)=N1] |
| 195 | 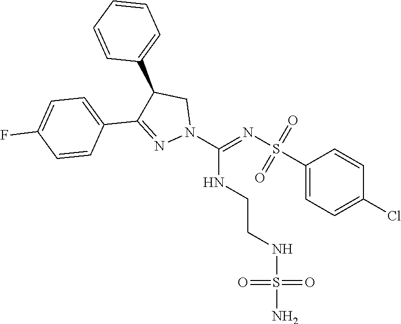 [SMILES: NS(=O)(=O)NCCN/C(=N\S(=O)(=O)c1ccc(Cl)cc1)N1C[C@H](c2ccccc2)C(c2ccc(F)cc2)=N1] |
| 196 | 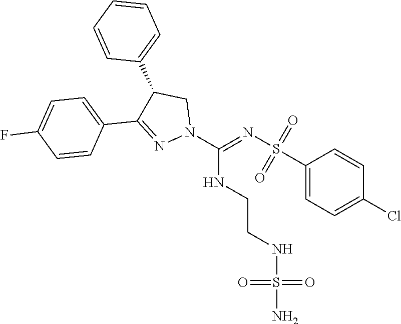 [SMILES: NS(=O)(=O)NCCN/C(=N\S(=O)(=O)c1ccc(Cl)cc1)N1C[C@@H](c2ccccc2)C(c2ccc(F)cc2)=N1] |
| 197 | 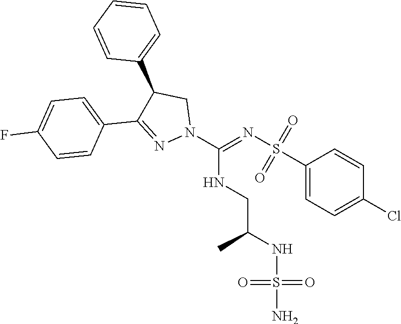 [SMILES: C[C@@H](CN/C(=N\S(=O)(=O)c1ccc(Cl)cc1)N1C[C@H](c2ccccc2)C(c2ccc(F)cc2)=N1)NS(N)(=O)=O] |
| 198 | 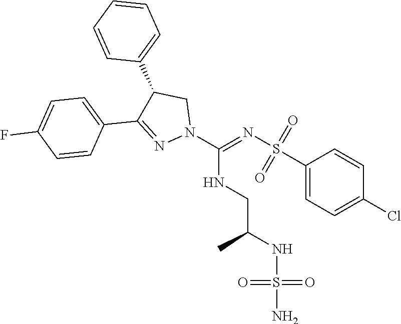 [SMILES: C[C@@H](CN/C(=N\S(=O)(=O)c1ccc(Cl)cc1)N1C[C@@H](c2ccccc2)C(c2ccc(F)cc2)=N1)NS(N)(=O)=O] |
| 199 | 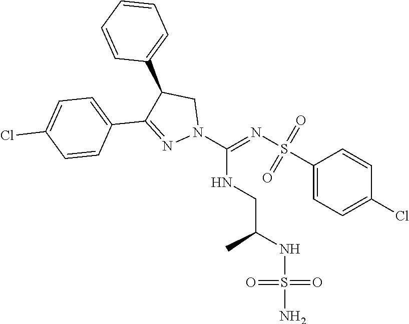 [SMILES: C[C@@H](CN/C(=N\S(=O)(=O)c1ccc(Cl)cc1)N1C[C@H](c2ccccc2)C(c2ccc(Cl)cc2)=N1)NS(N)(=O)=O] |
| 200 | 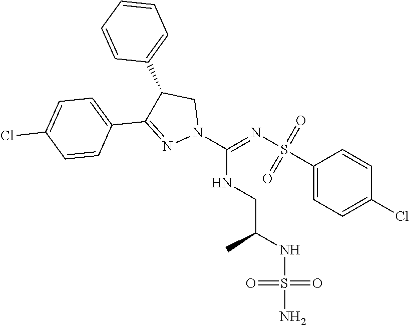 [SMILES: C[C@@H](CN/C(=N\S(=O)(=O)c1ccc(Cl)cc1)N1C[C@@H](c2ccccc2)C(c2ccc(Cl)cc2)=N1)NS(N)(=O)=O] |
| 201 | 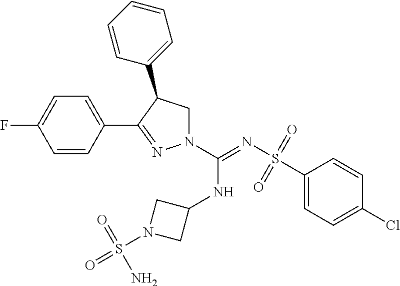 [SMILES: NS(=O)(=O)N1CC(N/C(=N\S(=O)(=O)c2ccc(Cl)cc2)N2C[C@H](c3ccccc3)C(c3ccc(F)cc3)=N2)C1] |
| 202 | 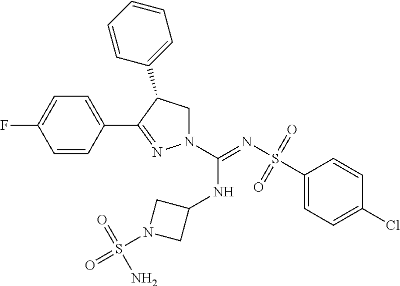 [SMILES: NS(=O)(=O)N1CC(N/C(=N\S(=O)(=O)c2ccc(Cl)cc2)N2C[C@@H](c3ccccc3)C(c3ccc(F)cc3)=N2)C1] |
| 203 | 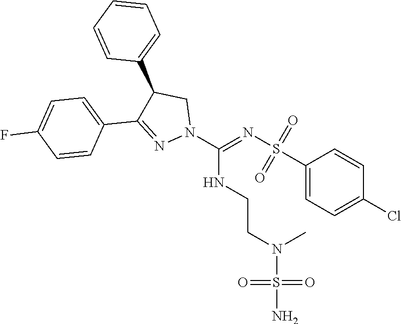 [SMILES: CN(CCN/C(=N\S(=O)(=O)c1ccc(Cl)cc1)N1C[C@H](c2ccccc2)C(c2ccc(F)cc2)=N1)S(N)(=O)=O] |
| 204 | 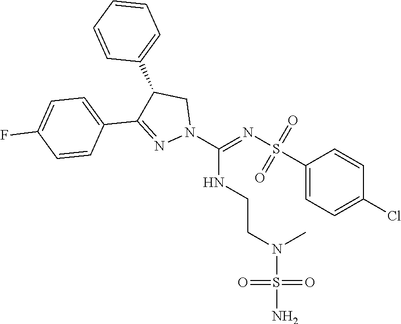 [SMILES: CN(CCN/C(=N\S(=O)(=O)c1ccc(Cl)cc1)N1C[C@@H](c2ccccc2)C(c2ccc(F)cc2)=N1)S(N)(=O)=O] |
| 205 | 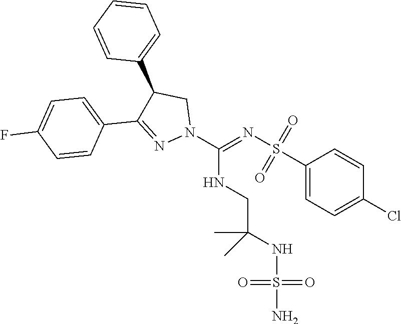 [SMILES: CC(C)(CN/C(=N\S(=O)(=O)c1ccc(Cl)cc1)N1C[C@H](c2ccccc2)C(c2ccc(F)cc2)=N1)NS(N)(=O)=O] |
| 206 | 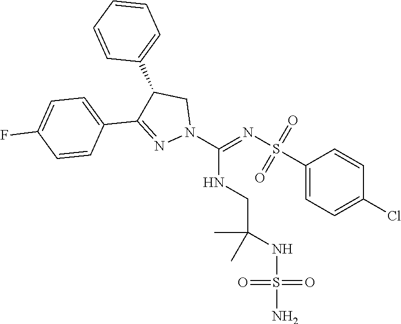 [SMILES: CC(C)(CN/C(=N\S(=O)(=O)c1ccc(Cl)cc1)N1C[C@@H](c2ccccc2)C(c2ccc(F)cc2)=N1)NS(N)(=O)=O] |
| 207 | 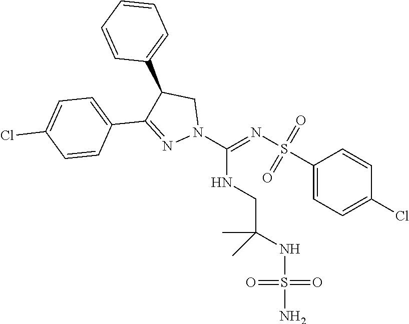 [SMILES: CC(C)(CN/C(=N\S(=O)(=O)c1ccc(Cl)cc1)N1C[C@H](c2ccccc2)C(c2ccc(Cl)cc2)=N1)NS(N)(=O)=O] |
| 208 | 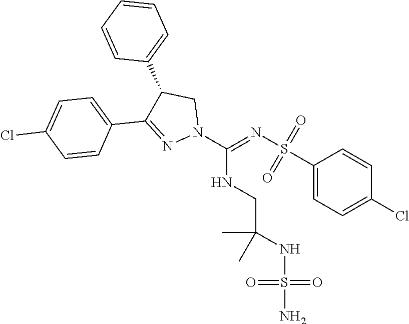 [SMILES: CC(C)(CN/C(=N\S(=O)(=O)c1ccc(Cl)cc1)N1C[C@@H](c2ccccc2)C(c2ccc(Cl)cc2)=N1)NS(N)(=O)=O] |
| 209 | 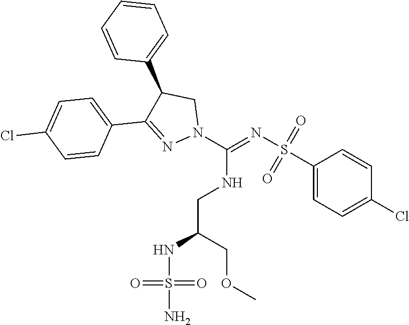 [SMILES: COC[C@H](CN/C(=N\S(=O)(=O)c1ccc(Cl)cc1)N1C[C@H](c2ccccc2)C(c2ccc(Cl)cc2)=N1)NS(N)(=O)=O] |
| 210 | 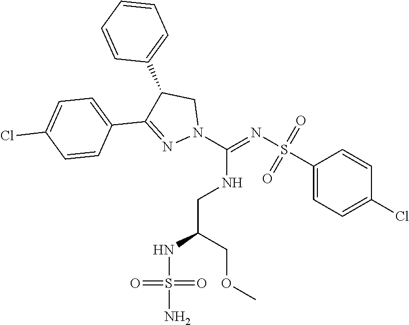 [SMILES: COC[C@H](CN/C(=N\S(=O)(=O)c1ccc(Cl)cc1)N1C[C@@H](c2ccccc2)C(c2ccc(Cl)cc2)=N1)NS(N)(=O)=O] |
| 211 | 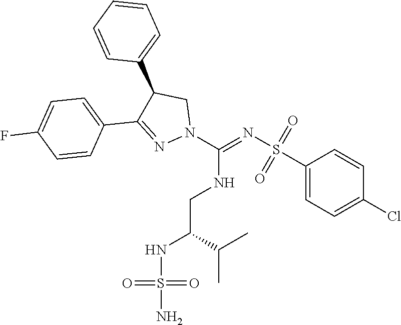 [SMILES: CC(C)[C@@H](CN/C(=N\S(=O)(=O)c1ccc(Cl)cc1)N1C[C@H](c2ccccc2)C(c2ccc(F)cc2)=N1)NS(N)(=O)=O] |
| 212 | 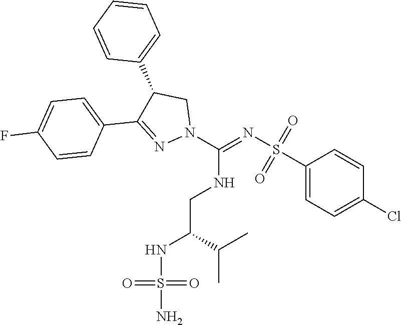 [SMILES: CC(C)[C@@H](CN/C(=N\S(=O)(=O)c1ccc(Cl)cc1)N1C[C@@H](c2ccccc2)C(c2ccc(F)cc2)=N1)NS(N)(=O)=O] |
| 213 | 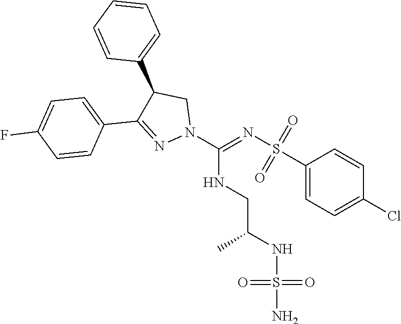 [SMILES: C[C@H](CN/C(=N\S(=O)(=O)c1ccc(Cl)cc1)N1C[C@H](c2ccccc2)C(c2ccc(F)cc2)=N1)NS(N)(=O)=O] |
| 214 | 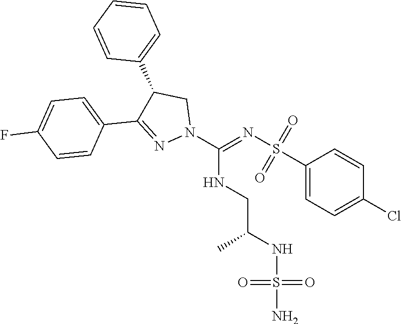 [SMILES: C[C@H](CN/C(=N\S(=O)(=O)c1ccc(Cl)cc1)N1C[C@@H](c2ccccc2)C(c2ccc(F)cc2)=N1)NS(N)(=O)=O] |
| 215 | 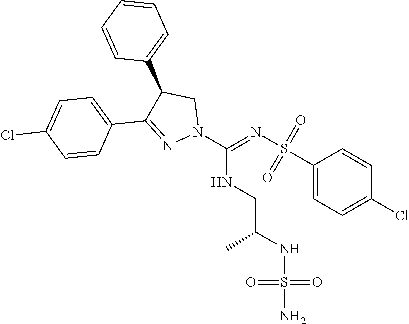 [SMILES: C[C@H](CN/C(=N\S(=O)(=O)c1ccc(Cl)cc1)N1C[C@H](c2ccccc2)C(c2ccc(Cl)cc2)=N1)NS(N)(=O)=O] |
| 216 | 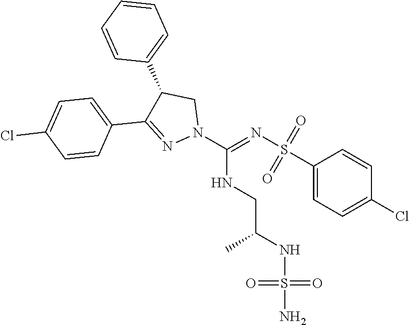 [SMILES: C[C@H](CN/C(=N\S(=O)(=O)c1ccc(Cl)cc1)N1C[C@@H](c2ccccc2)C(c2ccc(Cl)cc2)=N1)NS(N)(=O)=O] |
| 217 | 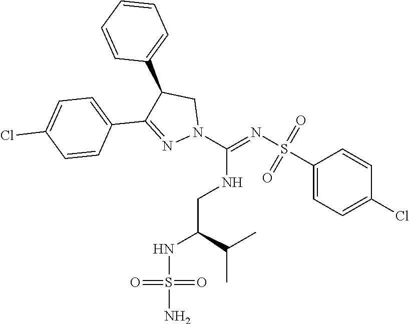 [SMILES: CC(C)[C@H](CN/C(=N\S(=O)(=O)c1ccc(Cl)cc1)N1C[C@H](c2ccccc2)C(c2ccc(Cl)cc2)=N1)NS(N)(=O)=O] |
| 218 | 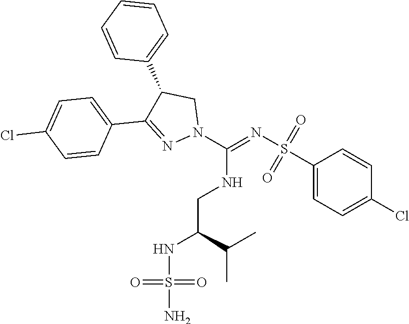 [SMILES: CC(C)[C@H](CN/C(=N\S(=O)(=O)c1ccc(Cl)cc1)N1C[C@@H](c2ccccc2)C(c2ccc(Cl)cc2)=N1)NS(N)(=O)=O] |
| 219 | 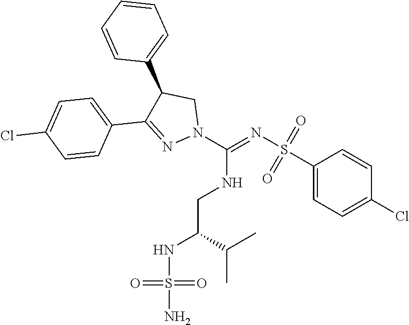 [SMILES: CC(C)[C@@H](CN/C(=N\S(=O)(=O)c1ccc(Cl)cc1)N1C[C@H](c2ccccc2)C(c2ccc(Cl)cc2)=N1)NS(N)(=O)=O] |
| 220 | 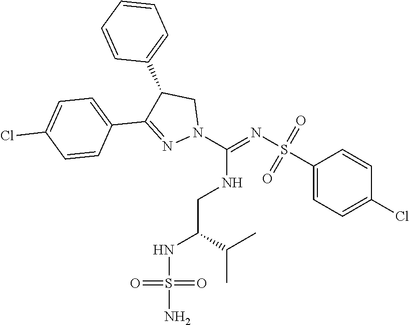 [SMILES: CC(C)[C@@H](CN/C(=N\S(=O)(=O)c1ccc(Cl)cc1)N1C[C@@H](c2ccccc2)C(c2ccc(Cl)cc2)=N1)NS(N)(=O)=O] |
| 221 | 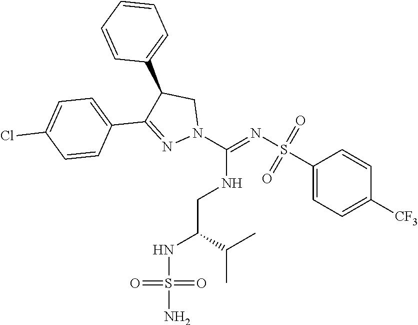 [SMILES: CC(C)[C@@H](CN/C(=N\S(=O)(=O)c1ccc(C(F)(F)F)cc1)N1C[C@H](c2ccccc2)C(c2ccc(Cl)cc2)=N1)NS(N)(=O)=O] |
| 222 | 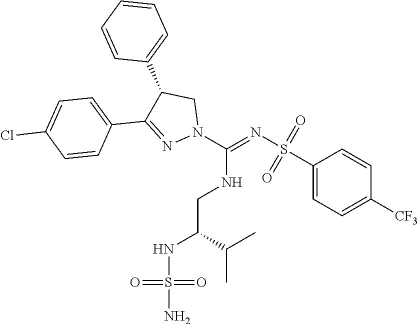 [SMILES: CC(C)[C@@H](CN/C(=N\S(=O)(=O)c1ccc(C(F)(F)F)cc1)N1C[C@@H](c2ccccc2)C(c2ccc(Cl)cc2)=N1)NS(N)(=O)=O] |
| 223 | 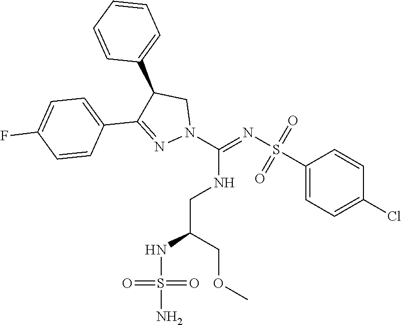 [SMILES: COC[C@H](CN/C(=N\S(=O)(=O)c1ccc(Cl)cc1)N1C[C@H](c2ccccc2)C(c2ccc(F)cc2)=N1)NS(N)(=O)=O] |
| 224 | 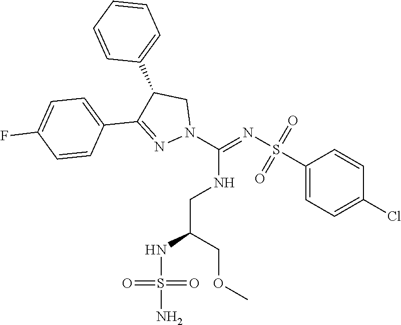 [SMILES: COC[C@H](CN/C(=N\S(=O)(=O)c1ccc(Cl)cc1)N1C[C@@H](c2ccccc2)C(c2ccc(F)cc2)=N1)NS(N)(=O)=O] |
| 225 | 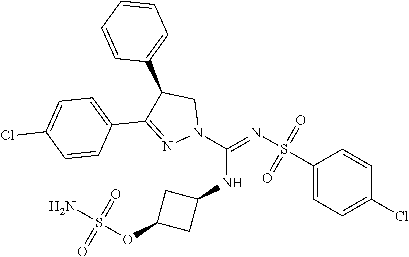 [SMILES: NS(=O)(=O)O[C@H]1C[C@@H](N/C(=N\S(=O)(=O)c2ccc(Cl)cc2)N2C[C@H](c3ccccc3)C(c3ccc(Cl)cc3)=N2)C1] |
| 226 | 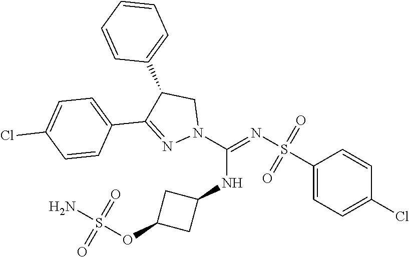 [SMILES: NS(=O)(=O)O[C@H]1C[C@@H](N/C(=N\S(=O)(=O)c2ccc(Cl)cc2)N2C[C@@H](c3ccccc3)C(c3ccc(Cl)cc3)=N2)C1] |
| 227 | 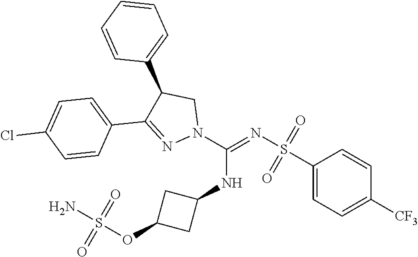 [SMILES: NS(=O)(=O)O[C@H]1C[C@@H](N/C(=N\S(=O)(=O)c2ccc(C(F)(F)F)cc2)N2C[C@H](c3ccccc3)C(c3ccc(Cl)cc3)=N2)C1] |
| 228 | 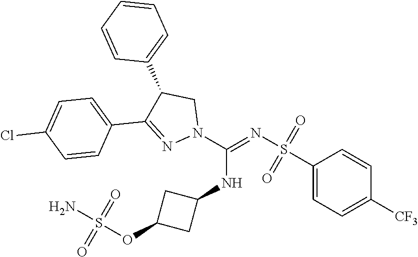 [SMILES: NS(=O)(=O)O[C@H]1C[C@@H](N/C(=N\S(=O)(=O)c2ccc(C(F)(F)F)cc2)N2C[C@@H](c3ccccc3)C(c3ccc(Cl)cc3)=N2)C1] |
| 229 | 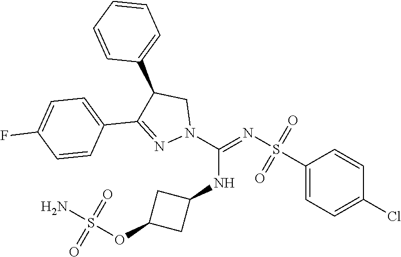 [SMILES: NS(=O)(=O)O[C@H]1C[C@@H](N/C(=N\S(=O)(=O)c2ccc(Cl)cc2)N2C[C@H](c3ccccc3)C(c3ccc(F)cc3)=N2)C1] |
| 230 | 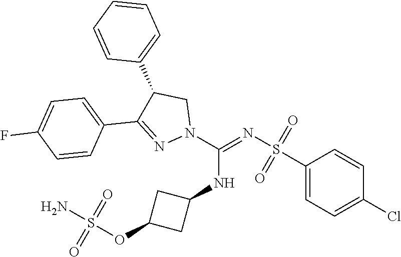 [SMILES: NS(=O)(=O)O[C@H]1C[C@@H](N/C(=N\S(=O)(=O)c2ccc(Cl)cc2)N2C[C@@H](c3ccccc3)C(c3ccc(F)cc3)=N2)C1] |
| 231 | 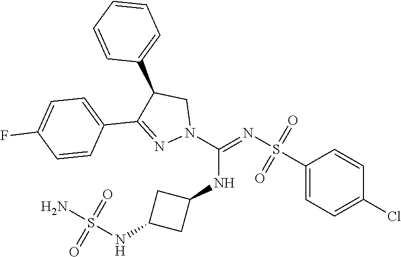 [SMILES: NS(=O)(=O)N[C@H]1C[C@H](N/C(=N\S(=O)(=O)c2ccc(Cl)cc2)N2C[C@H](c3ccccc3)C(c3ccc(F)cc3)=N2)C1] |
| 232 | 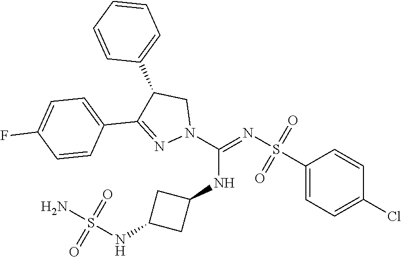 [SMILES: NS(=O)(=O)N[C@H]1C[C@H](N/C(=N\S(=O)(=O)c2ccc(Cl)cc2)N2C[C@@H](c3ccccc3)C(c3ccc(F)cc3)=N2)C1] |
| 233 | 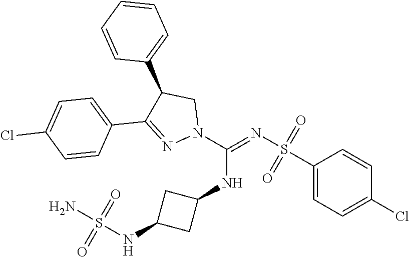 [SMILES: NS(=O)(=O)N[C@H]1C[C@@H](N/C(=N\S(=O)(=O)c2ccc(Cl)cc2)N2C[C@H](c3ccccc3)C(c3ccc(Cl)cc3)=N2)C1] |
| 234 | 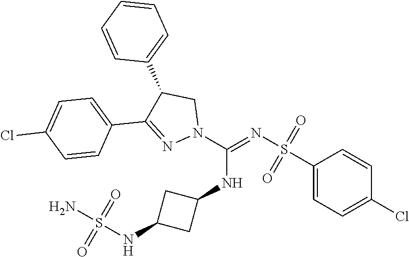 [SMILES: NS(=O)(=O)N[C@H]1C[C@@H](N/C(=N\S(=O)(=O)c2ccc(Cl)cc2)N2C[C@@H](c3ccccc3)C(c3ccc(Cl)cc3)=N2)C1] |
| 235 | 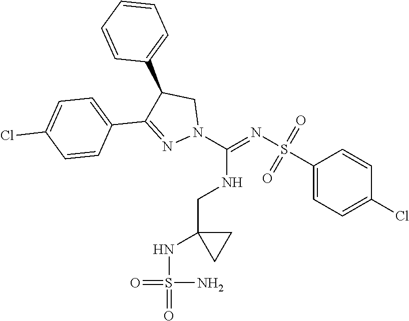 [SMILES: NS(=O)(=O)NC1(CN/C(=N\S(=O)(=O)c2ccc(Cl)cc2)N2C[C@H](c3ccccc3)C(c3ccc(Cl)cc3)=N2)CC1] |
| 236 | 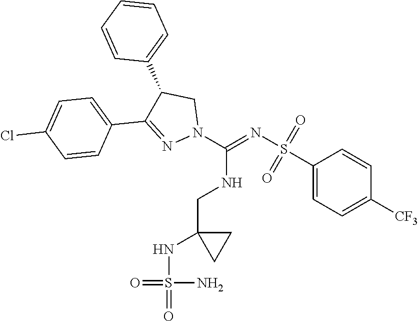 [SMILES: NS(=O)(=O)NC1(CN/C(=N\S(=O)(=O)c2ccc(C(F)(F)F)cc2)N2C[C@@H](c3ccccc3)C(c3ccc(Cl)cc3)=N2)CC1] |
| 237 | 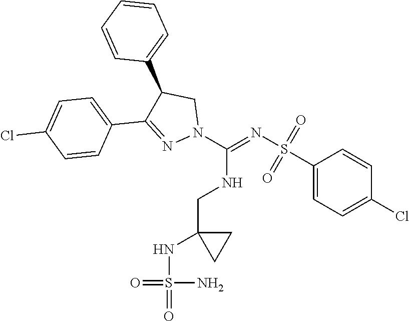 [SMILES: NS(=O)(=O)NC1(CN/C(=N\S(=O)(=O)c2ccc(Cl)cc2)N2C[C@H](c3ccccc3)C(c3ccc(Cl)cc3)=N2)CC1] |
| 238 | 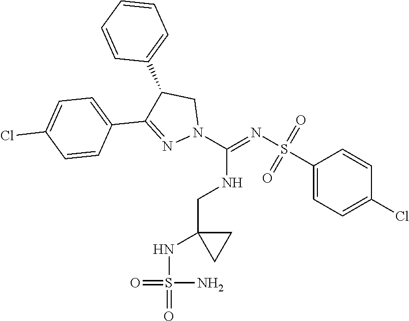 [SMILES: NS(=O)(=O)NC1(CN/C(=N\S(=O)(=O)c2ccc(Cl)cc2)N2C[C@@H](c3ccccc3)C(c3ccc(Cl)cc3)=N2)CC1] |
| 239 | 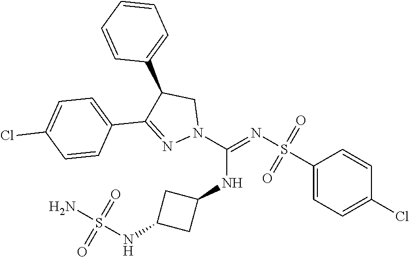 [SMILES: NS(=O)(=O)N[C@H]1C[C@H](N/C(=N\S(=O)(=O)c2ccc(Cl)cc2)N2C[C@H](c3ccccc3)C(c3ccc(Cl)cc3)=N2)C1] |
| 240 | 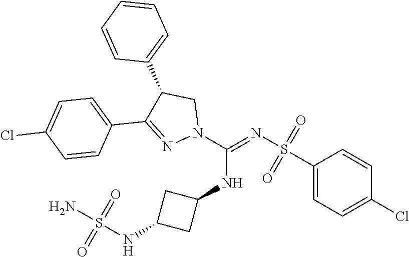 [SMILES: NS(=O)(=O)N[C@H]1C[C@H](N/C(=N\S(=O)(=O)c2ccc(Cl)cc2)N2C[C@@H](c3ccccc3)C(c3ccc(Cl)cc3)=N2)C1] |
| 241 | 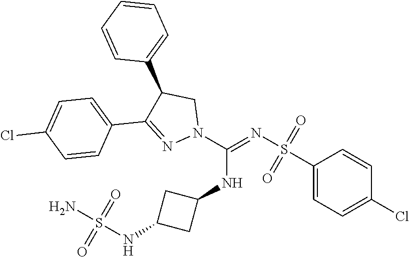 [SMILES: NS(=O)(=O)N[C@H]1C[C@H](N/C(=N\S(=O)(=O)c2ccc(Cl)cc2)N2C[C@H](c3ccccc3)C(c3ccc(Cl)cc3)=N2)C1] |
| 242 | 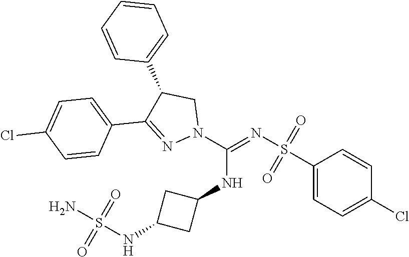 [SMILES: NS(=O)(=O)N[C@H]1C[C@H](N/C(=N\S(=O)(=O)c2ccc(Cl)cc2)N2C[C@@H](c3ccccc3)C(c3ccc(Cl)cc3)=N2)C1] |
| 243 | 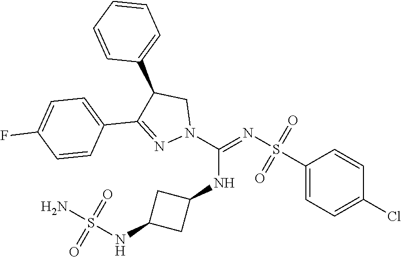 [SMILES: NS(=O)(=O)N[C@H]1C[C@@H](N/C(=N\S(=O)(=O)c2ccc(Cl)cc2)N2C[C@H](c3ccccc3)C(c3ccc(F)cc3)=N2)C1] |
| 244 | 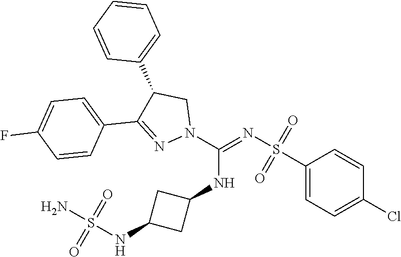 [SMILES: NS(=O)(=O)N[C@H]1C[C@@H](N/C(=N\S(=O)(=O)c2ccc(Cl)cc2)N2C[C@@H](c3ccccc3)C(c3ccc(F)cc3)=N2)C1] |
| 245 | 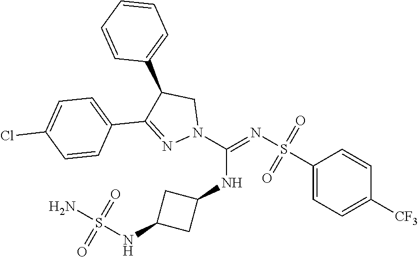 [SMILES: NS(=O)(=O)N[C@H]1C[C@@H](N/C(=N\S(=O)(=O)c2ccc(C(F)(F)F)cc2)N2C[C@H](c3ccccc3)C(c3ccc(Cl)cc3)=N2)C1] |
| 246 | 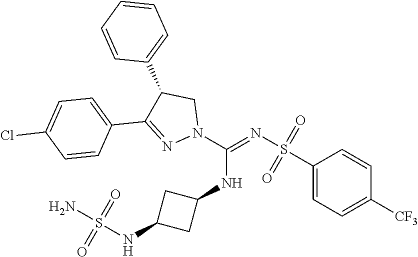 [SMILES: NS(=O)(=O)N[C@H]1C[C@@H](N/C(=N\S(=O)(=O)c2ccc(C(F)(F)F)cc2)N2C[C@@H](c3ccccc3)C(c3ccc(Cl)cc3)=N2)C1] |
| 247 | 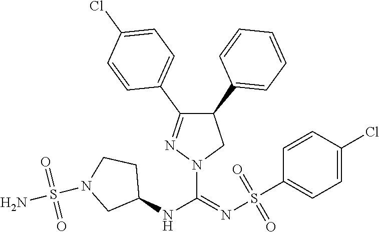 [SMILES: NS(=O)(=O)N1CC[C@@H](N/C(=N/S(=O)(=O)c2ccc(Cl)cc2)N2C[C@H](c3ccccc3)C(c3ccc(Cl)cc3)=N2)C1] |
| 248 | 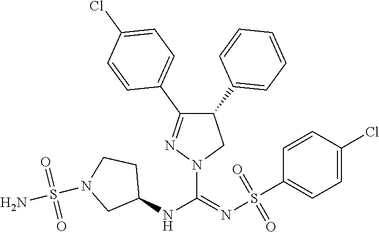 [SMILES: NS(=O)(=O)N1CC[C@@H](N/C(=N/S(=O)(=O)c2ccc(Cl)cc2)N2C[C@@H](c3ccccc3)C(c3ccc(Cl)cc3)=N2)C1] |
| 249 | 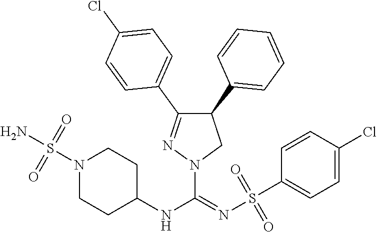 [SMILES: NS(=O)(=O)N1CCC(N/C(=N/S(=O)(=O)c2ccc(Cl)cc2)N2C[C@H](c3ccccc3)C(c3ccc(Cl)cc3)=N2)CC1] |
| 250 | 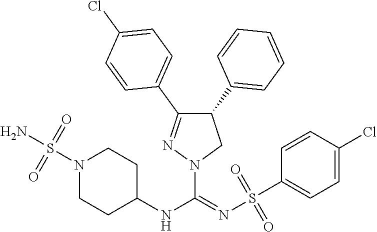 [SMILES: NS(=O)(=O)N1CCC(N/C(=N/S(=O)(=O)c2ccc(Cl)cc2)N2C[C@@H](c3ccccc3)C(c3ccc(Cl)cc3)=N2)CC1] |

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, any values provided in a range of values include both the upper and lower bounds, and any values contained within the upper and lower bounds.

A “compound of the present disclosure” and similar terms as used herein, whether explicitly noted or not, refers to CB1 antagonists or inverse agonists described herein, including compounds of Formula I and subformula thereof, and compounds of Table 1, as well as salts (e.g., pharmaceutically acceptable salts), solvates, hydrates, stereoisomers (including atropisomers), and tautomers thereof.

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

As used herein, the term “pharmaceutically acceptable salt” represents those salts of the compounds described that are suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid.

As used herein, the term “therapeutically effective amount” refers to an amount sufficient to effect beneficial or desired results, such as clinical results, and, as such, a “therapeutically effective amount” depends upon the context in which it is being applied.

As used herein, and as well understood in the art, “to treat” a condition or “treatment” of various diseases and disorders is an approach for obtaining beneficial or desired results, such as clinical results. Beneficial or desired results can include, but are not limited to, alleviation of one or more symptoms or conditions; diminishment of extent of disease, disorder, or condition; stabilizing (i.e., not worsening) state of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The term “subject,” as used herein, can be a human, non-human primate, or other mammal, such as but not limited to dog, cat, horse, cow, pig, goat, monkey, rat, mouse, and sheep. In preferred embodiments, the subject is a human.

As used herein, the term “pharmaceutical composition” refers to an active compound, formulated together with one or more pharmaceutically acceptable excipients. In some embodiments, a compound of the invention is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, or capsules; and parenteral administration, for example, by subcutaneous, intramuscular, or intravenous injection.

The term “pharmaceutically acceptable excipient,” as used herein, refers to any inactive ingredient (for example, a vehicle capable of suspending or dissolving the active compound) that is biocompatible and suitable for administration to a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium carbonate, calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials useful as excipients.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-8, 1-6, 1-4, or 1-2 carbon atoms (e.g., C₁-C₈, C₁-C₆, C₁-C₄, or C₁-C₂). Examples include, but are not limited to, methyl, ethyl, isobutyl, sec-butyl, tert-butyl, 2-methylpropyl, and 2,2-dimethylpropyl.

The term “alkynyl,” as used herein, refers to a branched or straight-chain monovalent aliphatic radical containing at least one carbon-carbon triple bond, and only C and H when unsubstituted. The monovalency of an alkynyl group does not include the optional substituents on the alkyl group. For example, if an alkynyl group is attached to a compound, the monovalency of the alkynyl group refers to its attachment to the compound and does not include any additional substituents that may be present on the alkynyl group. In some embodiments, the alkynyl group may contain, e.g., 2-8, 2-6, 2-4, or 2-3 carbon atoms (e.g., C₂-C₈, C₂-C₆, C₂-C₄, or C₂-C₃). Examples include, but are not limited to, ethynyl and propynyl.

The term “alkylene,” as used herein, refers to a divalent radical obtained by removing a hydrogen atom from a carbon atom of an alkyl group. The divalency of an alkylene group does not include the optional substituents on the alkylene group. Examples of alkylene groups include, but are not limited to, methylene, ethylene, and n-propylene.

The term “heterocycloalkyl,” as used herein, refers to a saturated non-aromatic monocyclic ring system having at least one heteroatom (e.g., N, O, or S) as a ring atom, and all other ring atoms are carbon atoms. A heterocyclyl ring may have five to ten ring atoms (e.g., five, six, seven, eight, nine, or ten), in which one or more (e.g., one, two, three, four, or five) ring atoms are heteroatoms independently selected from the group consisting of N, O, and S. For example, a heterocycloalkyl group may be a 5-membered ring (i.e., 5-membered heterocycle) containing one or more (e.g., one, two, three, or four) ring atoms that are heteroatoms independently selected from the group consisting of N, O, and S. As another example, a heterocycloalkyl group may be a 6-membered ring (i.e., 6-membered heterocycle) containing one or more (e.g., one, two, three, or four) ring atoms that are heteroatoms independently selected from the group consisting of N, O, and S. Examples of heterocycle groups include, but are not limited to pyrrolidine, thiolane, tetrahydrofuran, morpholine, piperidine, and piperazine, 2H-pyran, 4H-pyran, and tetrahydropyran.

The term “heteroaryl,” as used herein, refers to an aromatic monocyclic or fused ring bicyclic or multicyclic system having at least one heteroatom as a ring atom. For example, a heterocyclyl ring may have five to ten ring atoms (e.g., five, six, seven, eight, nine, or ten; i.e., 5-, 6-, 7-, 8-, 9-, or 10-membered heteroaryl), in which one or more (e.g., one, two, three, four, or five) ring atoms are heteroatoms independently selected from the group consisting of nitrogen, oxygen, and sulfur. Examples of heteroaryl groups include, but are not limited to pyrrole, pyrazole, isoxazole, imidazole, thiazole, thiophene, furan, diazole, triazole, tetrazole, oxazole, 1,3,4-oxadiazole, 1,3,4-thiadiazole, 1,2,3,4-oxatriazole, 1,2,3,4-thiatriazole, pyridine, pyrimidine, pyrazine, pyridazine, and triazine.

The phrase “optionally substituted X,” as used herein, is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The term “combination therapy” refers to a method of treatment including administering to a subject at least two therapeutic agents, optionally as one or more pharmaceutical compositions, as part of a therapeutic regimen. For example, a combination therapy may include administration of a single pharmaceutical composition including at least two therapeutic agents and one or more pharmaceutically acceptable carrier, excipient, diluent, or surfactant. A combination therapy may include administration of two or more pharmaceutical compositions, each composition including one or more therapeutic agent and one or more pharmaceutically acceptable carrier, excipient, diluent, or surfactant. The two or more agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due to an additive or synergistic effect of combining the two or more therapeutics.

As used herein, the term “CB1” or “CB1 receptor” refers to the cannabinoid receptor type 1. CB1 is a G protein-coupled cannabinoid receptor that in humans is encoded by the CNR1 gene. The human CB1 receptor is found predominantly in the brain and nervous system, as well as in peripheral organs and tissues. At least seven splice variants of the human CNR1 gene have been identified. CB1b is a splice variant of the CB1 receptor.

As used herein, the term “CB1_{b}” refers to a splice variant of the human CB1 receptor. CB1_{b} is preferentially expressed form of CB1 in β-cells and hepatocytes (e.g., particularly of obese individual), but has no significant expression in the brain. CB1_{b} is described, for example, in Patent Publication No. US20060115816. Selective inhibition of CB1 b in a CB1_{b}-associated disorder may decrease side-effects associated with CB1 inhibition in the brain (e.g., psychiatric and neurological side effects, including, depressed mood, anxiety, irritability, insomnia, dizziness, headache, seizures, and suicidal ideations).

### BRIEF DESCRIPTIONS OF THE DRAWINGS

FIG. 1 is a graph showing the body weight change in C57BL/6J mice on a high fat diet and administered Compound 9, semaglutide, tirzepatide, a combination of Compound 9 and semaglutide, and a combination of Compound 9 and tirzepatide.

FIG. 2 provides graphs showing the brain/plasma ratio of orally administered Compound 9 (10 mg/kg) in comparison to orally administered rimonabant (10 mg/kg) C57BL/6J in mice, as measured by area under the curve from 0 to 24 hours (AUC₀₋₂₄) and maximum concentration (Cₘₐₓ) post administration.

### DETAILED DESCRIPTION

Disclosed herein are compounds, pharmaceutical compositions, and the use of the pharmaceutical compositions for the treatment of a disorder modulated by the cannabinoid 1 (CB1) receptor in a subject.

### Compounds

The present disclosure provides compounds (e.g., CB1 receptor modulators) that are useful in the treatment of disorders mediated by the CB1 receptor. The compounds are generally described by formula (I):

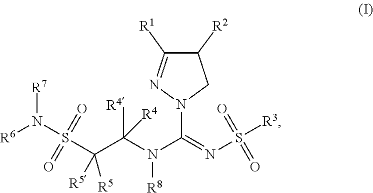

or a pharmaceutically acceptable salt thereof, in which R¹ is phenyl optionally substituted with one or more substituents selected from F, Cl, CN, and OCH₃; R² is C₁-C₆ alkyl or phenyl optionally substituted with F or CN; R³ is phenyl substituted with one or two substituents selected from F, Cl, CF₃, CN, OCH₃, C₂-C₆ alkynyl, and C(O)NH₂; or 5- or 6-membered heteroaryl containing 1-3 nitrogen atoms, in which the heteroaryl is optionally substituted with C₁-C₆ alkyl; R⁴, R^{4′}, R⁵, and R^{5′} are independently H or C₁-C₆ alkyl; R⁶ and R⁷ are independently H, OH, or C₁-C₆ alkyl; or R₆ and R₇, together with the nitrogen atom to which they are attached, form 5- or 6-membered heterocycloalkyl containing 1-2 nitrogen atoms, in which the heteroaryl isoptionally substituted with C₁-C₆ alkyl; and R⁸ is H or CH₃, provided that at least one of the following is true: (i) R¹ is not 4-chlorophenyl or 4-methoxyphenyl; (ii) R² is C₁-C₆ alkyl or phenyl substituted with CN; (iii) R³ is not 4-chlorophenyl, 3-cyanophenyl, or 3-methoxyphenyl; (iv) one of R⁴ and R⁵ is C₁-C₆ alkyl; (v) one of R⁶ and R⁷ is not H; and (vi) R⁸ is CH₃.

The compounds are also generally described by formula (II):

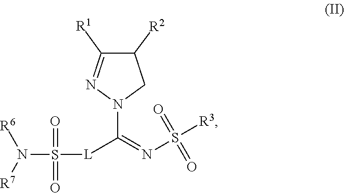

or a pharmaceutically acceptable salt thereof, in which R¹ is phenyl optionally substituted with one or more substituents selected from halogen, C₁-C₆ alkoxy, and CN; R² is C₁-C₆ alkyl, 5- or 6-membered heteroaryl, or phenyl optionally substituted with halogen or CN; R³ is phenyl substituted with one or more substituents selected from halogen, C₁-C₆ alkoxy, CF₃, CN, C₂-C₆ alkynyl, and C(O)NH₂; or 5- or 6-membered heteroaryl containing 1-3 nitrogen atoms, in which the heteroaryl is optionally substituted with C₁-C₆ alkyl; R⁶ and R⁷ are independently H, OH, or C₁-C₆ alkyl; or R₆ and R₇, together with the nitrogen atom to which they are attached, form 5- or 6-membered heterocycloalkyl containing 1-2 nitrogen atoms and optionally substituted with C₁-C₆ alkyl; L is

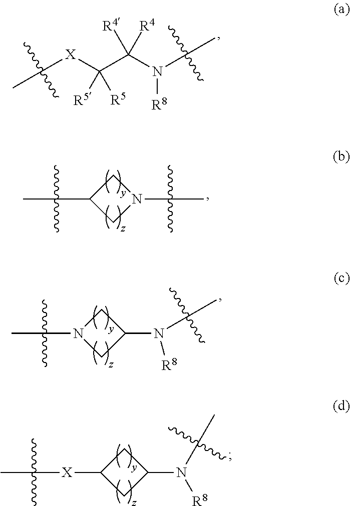

R⁸ is H or C₁-C₆ alkyl; R⁴, R^{4′}, R⁵, and R^{5′} are independently H or C₁-C₆ alkyl optionally substituted with C₁-C₆ alkoxy; X is O or NR⁹; R⁹ is H or C₁-C₆ alkyl; and y and z are independently 1, 2, or 3.

The compounds are also generally described by formula (III)::

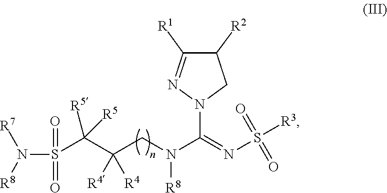

or a pharmaceutically acceptable salt thereof, in which R¹ is phenyl optionally substituted with one or more substituents selected from F, Cl, CN, and OCH₃; R² is C₁-C₆ alkyl, 5- or 6-membered heteroaryl, or phenyl optionally substituted with F or CN; R³ is phenyl substituted with one or more substituents selected from F, Cl, CF₃, CN, OCH₃, C₂-C₆ alkynyl, and C(O)NH₂; or 5- or 6-membered heteroaryl containing 1-3 nitrogen atoms, in which the heteroaryl is optionally substituted with C₁-C₆ alkyl; R⁴, R^{4′}, R⁵, and R^{5′} are independently H or C₁-C₆ alkyl; R⁶ and R⁷ are independently H, OH, or C₁-C₆ alkyl; or R₆ and R₇, together with the nitrogen atom to which they are attached, form 5- or 6-membered heterocycloalkyl containing 1-2 nitrogen atoms and optionally substituted with C₁-C₆ alkyl; R⁸ is H or C₁-C₆ alkyl; and n is 1, 2, or 3.

In some embodiments, the present disclosure features compounds which are inverse agonists of the CB1 receptor.

In some embodiments, the present disclosure features compounds which are antagonists of the CB1 receptor.

In some embodiments, the present disclosure features compounds that have increased affinity for the CB1 receptor, as compared to known CB1 modulators such as rimonabant, taranabant, and the compounds disclosed in International Publication WO2007106721A2, WO2007131219A2, WO2009033125A1, WO2009059264A1, WO2011044370A1, WO2012068529A2, and WO2014018695A1 (the contents of which are incorporated by reference herein in their entirety).

In some embodiments, the present disclosure features compounds that have increased selectivity for the CB1 receptor, as compared to known CB1 modulators such as rimonabant, taranabant, and the compounds disclosed in International Publication WO2007106721A2, WO2007131219A2, WO2009033125A1, WO2009059264A1, WO2011044370A1, WO2012068529A2, and WO2014018695A1.

In some embodiments, the present disclosure features compounds that have both increased affinity and increased selectivity for the CB1 receptor, as compared to known CB1 modulators such as rimonabant, taranabant, and the compounds disclosed in International Publication WO2007106721A2, WO2007131219A2, WO2009033125A1, WO2009059264A1, WO2011044370A1, WO2012068529A2, and WO2014018695A1.

In some embodiments, the present disclosure features compounds that exhibits reduced blood-brain-barrier penetration, as compared to known CB1 modulators such as rimonabant, taranabant, and the compounds disclosed in International Publication WO2007106721A2, WO2007131219A2, WO2009033125A1, WO2009059264A1, WO2011044370A1, WO2012068529A2, and WO2014018695A1.

In some embodiments, the present disclosure features compounds, when orally administered to mice at a dose of 10 mg/kg, exhibits a brain/plasma ratio in a subject of less than 0.2 (e.g., less than 0.15, less than 0.12, less than 0.1, less than 0.08, less than 0.06, or less than 0.05), as measured the area under the curve from 0 to 24 hours (AUC₀₋₂₄) post administration.

In some embodiments, the present disclosure features compounds, when orally administered to mice at a dose of 10 mg/kg, exhibits a brain/plasma ratio in a subject of less than 0.1 (e.g., less than 0.08, less than 0.06, less than 0.04, less than 0.02, or less than 0.01), as measured the maximum concentration (Cₘₐₓ) post administration.

In some embodiments, the present disclosure features compounds that have increase oral bioavailability, as compared to known CB1 modulators such as rimonabant, taranabant, and the compounds disclosed in International Publication WO2007106721A2, WO2007131219A2, WO2009033125A1, WO2009059264A1, WO2011044370A1, WO2012068529A2, and WO2014018695A1.

In some embodiments, the present disclosure feature compounds that have increased safety or efficacy profile, as compared to known CB1 modulators such as rimonabant, taranabant, and the compounds disclosed in International Publication WO2007106721A2, WO2007131219A2, WO2009033125A1, WO2009059264A1, WO2011044370A1, WO2012068529A2, and WO2014018695A1.

In some embodiments, the present disclosure feature compounds that result in lower risk (e.g., by at least 10%, by at least 20%, by at least 30%, by at least 40%, by at least 50%, by at least 60%, by at least 70%, by at least 80%, or by at least 90%) of psychiatric adverse events (e.g., suicidality) in treated subjects, as compared to subjects treated with known CB1 modulators such as rimonabant, taranabant, and the compounds disclosed in International Publication WO2007106721A2, WO2007131219A2, WO2009033125A1, WO2009059264A1, WO2011044370A1, WO2012068529A2, and WO2014018695A1.

In some embodiments, the present disclosure features compounds that have enhanced ability to reduce leptin levels, as compared to known CB1 modulators such as rimonabant, taranabant, and the compounds disclosed in International Publication WO2007106721A2, WO2007131219A2, WO2009033125A1, WO2009059264A1, WO2011044370A1, WO2012068529A2, and WO2014018695A1.

In some embodiments, the present disclosure features compounds that have increased binding or greater specificity for the CB1_{b} splice variant of the CB1 receptor, e.g., as compared to known CB1 modulators such as rimonabant, taranabant, and the compounds disclosed in International Publication WO2007106721A2, WO2007131219A2, WO2009033125A1, WO2009059264A1, WO2011044370A1, WO2012068529A2, and WO2014018695A1. In some embodiments, a compound of the disclosure binds to the human CB1 b with at least 2-fold, 3-fold, 4-fold, 5-fold, 6-fold, 7-fold, 8-fold, 9-fold, 10-fold, or 20-fold greater affinity that to the human CB1 receptor.

Exemplary compounds are shown in Table 1.

### Pharmaceutical Compositions

A pharmaceutical composition of the present disclosure contains one or more of the compounds disclosed herein (e.g., one or more of the compounds of formulas (I), (IA), (IB), (IIA), (IIB), (IIIA), (IIIB), (II), (IVA), (VA), (VIA), (IVB), (VB), (VIB), and (Ill), and Table 1) as the therapeutic compound. In addition to a therapeutically effective amount of the compound, the pharmaceutical compositions also contain a pharmaceutically acceptable excipient, which can be formulated by methods known to those skilled in the art. In some embodiments, the pharmaceutical compositions for treating cancer contain one or more of the compounds disclosed herein (e.g., one or more of the compounds of formulas (I), (IA), (IB), (IIA), (IIB), (IIIA), (IIIB), (II), (IVA), (VA), (VIA), (IVB), (VB), (VIB), and (Ill), and Table 1) may be formulated and/or administered with or without other therapeutics for a particular condition. Examples of such therapeutics (second therapeutic agents) are described herein.

The compounds disclosed herein (e.g., the compounds of formulas (I), (IA), (IB), (IIA), (IIB), (IIIA), (IIIB), (II), (IVA), (VA), (VIA), (IVB), (VB), (VIB), and (Ill), and Table 1) may be used in the form of free base, or in the form of salts, and as solvates. All forms are within the scope of the disclosure.

Exemplary routes of administration of the pharmaceutical compositions (or the compounds of the composition) include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

### Formulations for Oral Administration

The pharmaceutical compositions of the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Pharmaceutical compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Parenteral Administration

The pharmaceutical compositions of the invention can be administered in a pharmaceutically acceptable parenteral (e.g., intravenous, intramuscular, subcutaneous or the like) formulation as described herein. The pharmaceutical composition may also be administered parenterally in dosage forms or formulations containing conventional, non-toxic pharmaceutically acceptable carriers and adjuvants. In particular, formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water; water adjusted to a suitable pH by addition of an appropriate amount of hydrochloric acid, sodium hydroxide, or a suitable buffer; 1,3-butanediol; Ringer's solution; and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference in its entirety.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropyl cellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 23^{rd} Ed., Adejare, Ed., Academic Press (2020) and in The United States Pharmacopeia and National Formulary (USP-NF 2021 Issues 1-3), published in 2021.

Formulations for parenteral administration may, for example, contain sterile water, saline, polyalkylene glycols (e.g., polyethylene glycol), oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain, for example, lactose, or may be aqueous solutions containing, for example, polyoxyethylene-9-lauryl ether, glycocholate and deoxycholate, or may be oily solutions for administration in the form of nasal drops, or as a gel.

### Methods of Use

Without wishing to be bound by theory, the present disclosure is based, in part, on the discovery that compounds of the present disclosure are peripherally restricted (i.e., have an inability or limited ability to cross the blood-brain-barrier or are readily eliminated from the brain through active transport systems) and thus produce no or limited CNS effects. Thus, the compounds of the present disclosure can provide peripherally mediated efficacy in treating CB1 modulated disorders, such as diabetic disorders, dyslipidemia disorders, cardiovascular disorders, inflammatory disorders, hepatic disorders, cancers, and obesity and co-morbidities thereof with improved treatment safety, e.g., with respect to CNS effects.

Accordingly, in some embodiments, the present disclosure provides a method of treating a diabetic disorder in a subject (e.g., a human) with a compound of the present disclosure (e.g., a compound of formula (I), (IA), (IB), (IIA), (IIB), (IIIA), (IIIB), (II), (IVA), (VA), (VIA), (IVB), (VB), (VIB), or (III), or a compound of Table 1) or a pharmaceutically acceptable salt thereof. Examples of diabetic disorders include, but are not limited to, Type 1 diabetes, Type 2 diabetes, inadequate glucose tolerance, and insulin resistance. In some embodiments, the diabetic disorder is Type 1 diabetes. In some embodiments, the diabetic disorder is Type 2 diabetes. In some embodiments, the diabetic disorder is inadequate glucose tolerance. In some embodiments, the diabetic disorder is Type 1 diabetes. In some embodiments, the diabetic disorder is inadequate glucose intolerance. In some embodiments, the diabetic disorder is insulin resistance.

In some embodiments, the present disclosure provides a method of treating a dyslipidemia disorder in a subject (e.g., a human) with a compound of the present disclosure (e.g., a compound of formula (I), (IA), (IB), (IIA), (IIB), (IIIA), (IIIB), (II), (IVA), (VA), (VIA), (IVB), (VB), (VIB), or (III), or a compound of Table 1) or a pharmaceutically acceptable salt thereof. Examples of dyslipidemia disorders include, but are not limited to, undesirable blood lipid levels, low levels of high-density lipoprotein, high levels of low-density lipoprotein, high levels of triglycerides, and combinations thereof. In some embodiments, the dyslipidemia disorder is undesirable blood lipid levels. In some embodiments, the dyslipidemia disorder is low levels of high-density lipoprotein. In some embodiments, the dyslipidemia disorder is high levels of low-density lipoprotein. In some embodiments, the dyslipidemia disorder is undesirable blood lipid levels.

In some embodiments, the present disclosure provides a method of treating a cardiovascular disorder in a subject (e.g., a human) with a compound of the present disclosure (e.g., a compound of formula (I), (IA), (IB), (IIA), (IIB), (IIIA), (IIIB), (II), (IVA), (VA), (VIA), (IVB), (VB), (VIB), or (III), or a compound of Table 1) or a pharmaceutically acceptable salt thereof. Examples of cardiovascular disorders include, but are not limited to, atherosclerosis, hypertension, stroke, and heart attack. In some embodiments, the cardiovascular disorder is atherosclerosis. In some embodiments, the cardiovascular disorder is hypertension. In some embodiments, the cardiovascular disorder is stroke. In some embodiments, the cardiovascular disorder is heart attack.

In some embodiments, the present disclosure provides a method of treating an inflammatory disorder in a subject (e.g., a human) with a compound of the present disclosure (e.g., a compound of formula (I), (IA), (IB), (IIA), (IIB), (IIIA), (IIIB), (II), (IVA), (VA), (VIA), (IVB), (VB), (VIB), or (III), or a compound of Table 1) or a pharmaceutically acceptable salt thereof. Examples of inflammatory disorders include, but are not limited to, osteoarthritis, rheumatoid arthritis, inflammatory bowel diseases, and obesity-associated inflammation. In some embodiments, the inflammatory disorder is osteoarthritis. In some embodiments, the inflammatory disorder is osteoarthritis. In some embodiments, the inflammatory disorder is rheumatoid arthritis. In some embodiments, the inflammatory disorder is an inflammatory bowel disease. In some embodiments, the disorder is obesity-associated inflammation.

In some embodiments, the present disclosure provides a method of treating a hepatic disorder in a subject (e.g., a human) with a compound of the present disclosure (e.g., a compound of formula (I), (IA), (IB), (IIA), (IIB), (IIIA), (IIIB), (II), (IVA), (VA), (VIA), (IVB), (VB), (VIB), or (III), or a compound of Table 1) or a pharmaceutically acceptable salt thereof. Examples of hepatic disorders include, but are not limited to liver inflammation, liver fibrosis, non-alcoholic steatohepatitis, fatty liver, enlarged liver, alcoholic liver disease, jaundice, cirrhosis, and hepatitis. In some embodiments, the hepatic disorder is liver inflammation. In some embodiments, the hepatic disorder is liver fibrosis. In some embodiments, the hepatic disorder is non-alcoholic steatohepatitis. In some embodiments, the hepatic disorder is fatty liver. In some embodiments, the hepatic disorder is enlarged liver. In some embodiments, the hepatic disorder is alcoholic liver disease. In some embodiments, the hepatic disorder is jaundice. In some embodiments, the hepatic disorder is cirrhosis. In some embodiments, the hepatic disorder is hepatitis.

In some embodiments, the present disclosure provides a method of treating cancer in a subject (e.g., a human) with a compound of the present disclosure (e.g., a compound of formula (I), (IA), (IB), (IIA), (IIB), (IIIA), (IIIB), (II), (IVA), (VA), (VIA), (IVB), (VB), (VIB), or (III), or a compound of Table 1) or a pharmaceutically acceptable salt thereof. Examples of cancers include, but are not limited to, colon cancer, breast cancer, thyroid cancer, alveolar rhabdomyosarcoma, and hepatocellular carcinoma. In some embodiments, the cancer is colon cancer. In some embodiments, the cancer is breast cancer. In some embodiments, the cancer is thyroid cancer. In some embodiments, the cancer is alveolar rhabdomyosarcoma. In some embodiments, the cancer is hepatocellular carcinoma.

In some embodiments, the present disclosure provides a method of treating obesity or a co-morbidity thereof in a subject (e.g., a human) with a compound of the present disclosure (e.g., a compound of formula (I), (IA), (IB), (IIA), (IIB), (IIIA), (IIIB), (II), (IVA), (VA), (VIA), (IVB), (VB), (VIB), or (III), or a compound of Table 1) or a pharmaceutically acceptable salt thereof. Examples of co-morbidities of obesity include, but are not limited to, hypertension; gallbladder disease; gastrointestinal disorders; menstrual irregularities; degenerative arthritis; venous statis ulcers; pulmonary hypoventilation syndrome; sleep apnea; snoring; coronary artery disease; arterial sclerotic disease; pseudotumor cerebri; accident proneness; increased risks with surgeries; osteoarthritis; high cholesterol; or increased incidence of malignancy of the ovaries, cervix, uterus, breasts, prostrate, or gallbladder. In some embodiments, the co-morbidity of obesity is hypertension. In some embodiments, the co-morbidity of obesity is gallbladder disease. In some embodiments, the co-morbidity of obesity is a gastrointestinal disorder. In some embodiments, the co-morbidity of obesity is menstrual irregularities. In some embodiments, the co-morbidity of obesity is degenerative arthritis. In some embodiments, the co-morbidity is venous statis ulcers. In some embodiments, the co-morbidity of obesity is pulmonary hypoventilation syndrome. In some embodiments, the co-morbidity of obesity is sleep apnea. In some embodiments, the co-morbidity of obesity is snoring. In some embodiments, the co-morbidity of obesity is coronary artery disease. In some embodiments, the co-morbidity of obesity is arterial sclerotic disease. In some embodiments, the co-morbidity of obesity is pseudotumor cerebri. In some embodiments, the co-morbidity of obesity is accident proneness. In some embodiments, the co-morbidity of obesity is increased risks with surgeries.

In some embodiments, the co-morbidity of obesity is osteoarthritis. In some embodiments, the co-morbidity of obesity is high cholesterol. In some embodiments, the co-morbidity of obesity is increased incidence of malignancy of the ovaries, cervix, uterus, breasts, prostrate, or gallbladder. The dosage of the compound of the disclosure depends on factors including the route of administration, the disease to be treated, and physical characteristics, e.g., age, weight, and general health, of the subject. Typically, the amount of a compound disclosed herein (e.g., a compound of formula (I), (IA), (IB), (IIA), (IIB), (IIIA), (IIIB), (II), (IVA), (VA), (VIA), (IVB), (VB), (VIB), and (III), or Table 1) contained within a single dose may be an amount that effectively treats the disease without inducing significant toxicity. Pharmaceutical compositions of the disclosure that contain one or more of the compounds disclosed herein (e.g., one or more of the compounds of formulas (I), (IA), (IB), (IIA), (IIB), (IIIA), (IIIB), (II), (IVA), (VA), (VIA), (IVB), (VB), (VIB), and (III), and Table 1) may be administered to a subject in need thereof one or more times daily, or as medically necessary.

### Combination Therapy

In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (III), or Table 1) or a pharmaceutically acceptable salt thereof is administered with a second therapeutic agent, e.g., a therapeutic agent that is suitable for treating any of the disorders described herein.

Examples of second therapeutic agents suitable for administration with a compound of the disclosure (e.g., a compound of formula (I), (IA), (IB), (IIA), (IIB), (IIIA), (IIIB), (II), (IVA), (VA), (VIA), (IVB), (VB), (VIB), or (III), or Table 1) or a pharmaceutically acceptable salt thereof include, but are not limited to, PPAR-γ agonists (e.g., glitazones such as troglitazone, pioglitazone, englitazone, MCC-555, and rosiglitazone), biguanides (e.g., metformin and phenformin), insulin or insulin mimetics, sulfonylureas (e.g., tolbutamide or glipizide), α-glucosidase inhibitors (e.g., acarbose), HMG-CoA reductase inhibitors (e.g., lovastatin, simvastatin, pravastatin, fluvastatin, atorvastatin, or rivastatin), sequestrants (e.g., cholestyramine, colestipol, or dialkylaminoalkyl derivatives of crosslinked dextran), nicotinyl alcohol, nicotinic acid or salts thereof, PPAR-α agonists (e.g., fenofibric acid derivatives such as gemfibrozil, clofibrate, fenofibrate, and bezafibrate), inhibitors of cholesterol absorption, acyl CoA:cholesterol acyltransferase inhibitors, probucol, PPAR-α/γ agonists, ileal bile acid transporter inhibitors, insulin receptor activators, dipeptidyl peptidase IV inhibitors, exenatide, pramlintide, FBPase inhibitors, glucagon receptor antagonists, glucagon-like peptide 1, glucagon-like peptide 1 receptor agonists (e.g., liraglutide, semaglutide, exenatide, lixisenatide, dulaglutide, and tirzepatide), growth hormone secretagogues, growth hormone secretagogue receptor agonists, growth hormone secretagogue receptor antagonists, melanocortin agonists, melanocortin 4 receptor agonists, beta-3 agonists, serotonin receptor 2C agonists, orexin antagonists, melanin concentrating hormone 1 antagonists, melanin concentrating hormone 2 agonists, melanin concentrating hormone 2 antagonists, galanin antagonists, CCK agonists, CCK-A agonists, corticotropin-releasing hormone agonists, NPY 5 antagonists, NPY 1 antagonists, histamine receptor-3 modulators, histamine receptor-3 blockers, p-hydroxy steroid dehydrogenase-1 inhibitors, phosphodiesterase inhibitors, phosphodiesterase-3B inhibitors, norepinephrine transport inhibitors, non-selective serotonin/norepinephrine transport inhibitors (e.g., sibutramine, phentermine, or fenfluramine), ghrelin antagonists, leptin derivatives, bombesin receptor subtype 3 agonists, ciliary neurotrophic factor or derivatives thereof (e.g., axokine), monoamine reuptake inhibitors, uncoupling protein-1 activators, uncoupling protein-2 activators, uncoupling protein-3 activators, thyroid hormone beta agonists, fatty acid synthase inhibitors, diacylglycerol acetyltransferase 2 inhibitors, acetyl-CoA carboxylase-2 inhibitors, glucocorticoid antagonists, acyl-estrogens, lipase inhibitors (e.g., orlistat), fatty acid transporter inhibitors, dicarboxylate transporter inhibitors, glucose transporter inhibitors, sodium-glucose co-transporters, phosphate transporter inhibitors, serotonin reuptake inhibitors, thiazolidinediones, Metformin, Topiramate, opiate antagonists (e.g., naltrexone), non-selective transport inhibitors (e.g., bupropion), or MAO inhibitors (e.g., Moclobemide, Brofaromine, BW A616U, Ro 41-1049, RS-2232, SR 95191, Harmaline, Harman, Amiflamine, BW 1370U87, FLA 688, FLA 788; Bifemelane, Clorgyline, LY 51641, MDL 72,394, 5-(4-benzyloxyphenyl)-3-(2-cyanoethyl)-(3H)-1,3,4-oxadiazol-2-one, 5-(4-arylmethoxyphenyl)-2-(2-cyanoethyl)tetrazoles, Lazabemide, Ro 16-6491, Almoxatone, XB308, RS-1636, RS-1653, NW-1015, SL 340026, L-selegiline, Rasagiline, Pargyline, AGN 1135, MDL 72,974, MDL 72,145, MDL 72,638, LY 54761, MD 780236, MD 240931, Bifemelane, Toloxatone, Cimoxatone, Iproniazid, Phenelzine, Nialamide, phenylhydrazine, 1-phenylcyclopropylamine, Isocarboxazid, or Tranylcypromine).

In some embodiments, a compound of the disclosure (e.g., a compound of formula (I), (IA), (IB), (IIA), (IIB), (IIA), (IIIB), (II), (IVA), (VA), (VIA), (IVB), (VB), (VIB), or (III), or Table 1) or a pharmaceutically acceptable salt thereof is administered with a glucagon-like peptide 1 analog. In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (III), or Table 1) or a pharmaceutically acceptable salt thereof is administered with semaglutide.

In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (III), or Table 1) or a pharmaceutically acceptable salt thereof is administered with a gastric inhibitory polypeptide analog. In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (Ill), or Table 1) or a pharmaceutically acceptable salt thereof is administered with tirzepatide.

In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (Ill), or Table 1) or a pharmaceutically acceptable salt thereof is administered with a glucagon-like peptide 1 analog. In some embodiments, a compound of the disclosure (e.g., a compound of formula (I), (IA), (IB), (IIA), (IIB), (IIIA), or (IIIB), or Table 1) or a pharmaceutically acceptable salt thereof is administered with liraglutide.

In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (Ill), or Table 1) or a pharmaceutically acceptable salt thereof is administered with a glucagon-like peptide 1 analog. In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (Ill), or Table 1) or a pharmaceutically acceptable salt thereof is administered with exenatide.

In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (Ill), or Table 1) or a pharmaceutically acceptable salt thereof is administered with a glucagon-like peptide 1 analog. In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (Ill), or Table 1) or a pharmaceutically acceptable salt thereof is administered with lixisenatide.

In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (Ill), or Table 1) or a pharmaceutically acceptable salt thereof is administered with a glucagon-like peptide 1 analog. In some embodiments, a compound of the disclosure (e.g., a compound of formula (I), (IA), (IB), (IIA), (IIB), (IIIA), (IIIB), (II), (IVA), (VA), (VIA), (IVB), (VB), (VIB), or (Ill), or Table 1) or a pharmaceutically acceptable salt thereof is administered with dulaglutide.

### EXAMPLES

### Example 1. Synthesis of Intermediates

### Synthesis of (S)-2-aminopropane-1-sulfonamide (I1)

### Synthesis of (S)-2-(((benzyloxy)carbonyl)amino)propyl methanesulfonate (I1.1)

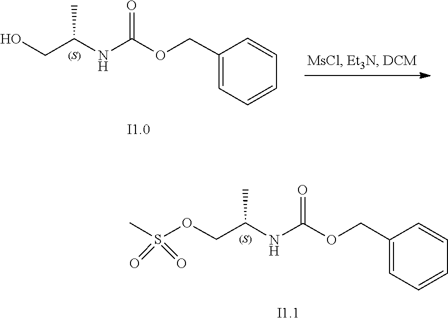

Methanesulfonyl chloride (1.92 mL, 24.8 mmol, 1.04 eq.) in dichloromethane (48 mL) was added to a solution of benzyl (S)-(1-hydroxypropan-2-yl)carbamate (5.0 g, 23.9 mmol, 11.0) and triethylamine (3.7 mL, 26.3 mmol, 1.1 eq.) in dichloromethane (96 mL) at 0° C. for 30 min. After stirring at room temperature for 16 hrs, the reaction mixture was washed with sat. aqueous sodium hydrogen bicarbonate (80 mL) and brine (80 mL). The organic extract was dried over magnesium sulfate, filtered and concentrated in vacuo to dryness to afford the expected product as a white solid (6.85 g, 23.8 mmol, 99% yield). LC-MS (ESI+): 288.1 (M+H⁺).

### Synthesis of (S)-S-(2-(((benzyloxy)carbonyl)amino)propyl) ethanethioate (I1.2)

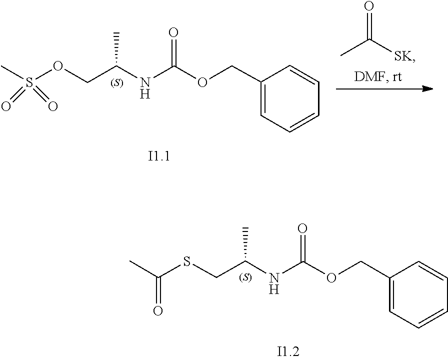

Potassium thioacetate (13.6 g, 119.2 mmol, 5.0 eq.) was added to a stirred solution of (S)-2-(((benzyloxy)carbonyl)amino)propyl methanesulfonate (6.85 g, 23.8 mmol, 11.1) in dimethylformamide (120 mL). After stirring at room temperature for 18 hours, solvent was partially removed under reduced pressure, diluted with ethyl acetate (100 mL) and washed with water (80 mL). The collected organic phase was washed with brine (60 mL), dried over magnesium sulfate, filtered and concentrated in vacuo to give a dark oil which was purified by flash chromatography on silica eluting with ethyl acetate/heptane mixtures (0/100 to 50/50, v/v) to give a brownish solid (6.3 g, 23.7 mmol, 99% yield). LC-MS (ESI+): 268.1 (M+H⁺).

### Synthesis of benzyl (S)-(1-(chlorosulfonyl)propan-2-yl)carbamate (I1.3)

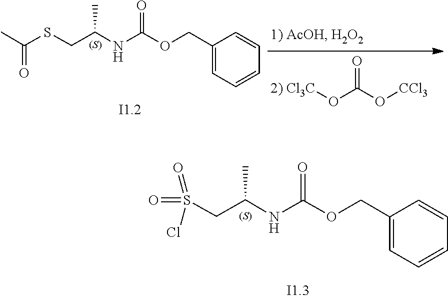

A solution of hydrogen peroxide 50% (13.5 mL, 0.63 mL/mmol) in acetic acid (49 mL) was added dropwise to a solution of (S)-S-(2-(((benzyloxy)carbonyl)amino)propyl) ethanethioate (5.73 g, 21.4 mmol, 11.2) in acetic acid (36 mL). After stirring at room temperature for 18 hours, palladium on activated carbon was added to destroy excess peroxide and the resulting mixture was filtered through celite pad. The filtrate was concentrated in vacuo to dryness.

The brown oil obtained was taken in dichloromethane (120 mL) and triphosgene (8.9 g, 30.0 mmol, 1.4 eq.) was added followed up by dimethylformamide (3.0 mL). After stirring at room temperature for 18 hours, the solvents were removed under reduced pressure and the residue purified on silica eluting with dichloromethane/methanol mixtures (0/100 to 10/90, v/v) to give a beige solid (5.43 g, 18.7 mmol, 87% yield). LC-MS (ESI+): 292.0 (M+H⁺).

### Synthesis of benzyl (S)-(1-sulfamoylpropan-2-yl)carbamate (I1.4)

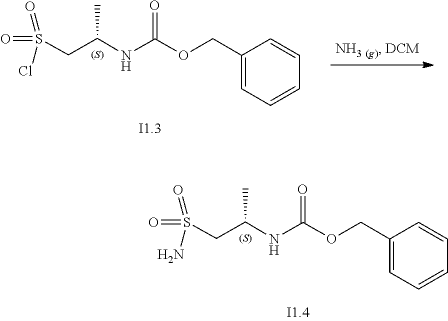

Ammonia gas was bubbled through a stirred solution of benzyl (S)-(1-(chlorosulfonyl)propan-2-yl)carbamate (5.4 g, 18.5 mmol, 11.3) in dichloromethane (185 mL) at room temperature for 15 min. Upon completion, the reaction mixture was concentrated in vacuo to dryness and the white pale solid obtained was used in the next step without further purification (5.0 g, 18.4 mmol, 99% yield). LC-MS (ESI+): 273.2 (M+H⁺).

### Synthesis of (S)-2-aminopropane-1-sulfonamide (I1)

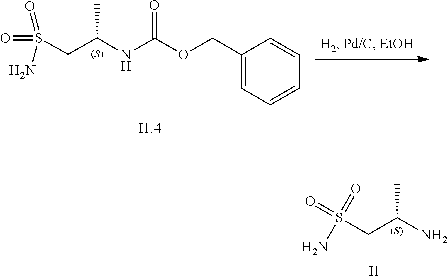

To a solution of benzyl (S)-(1-sulfamoylpropan-2-yl)carbamate (5.3 g, 19.5 mmol, 11.4) in ethanol (195 mL) with palladium 10% on carbon powder (Pd/C, 10 wt. %) (2.0 g, 0.1 eq.) first degassed in vacuo and then saturated with hydrogen gas, repeated 3 times. After stirring overnight at room temperature under hydrogen atmosphere, the mixture was filtered through celite pad and concentrated in vacuo to yield the expected compound as a brownish white solid (2.65 g, 98% yield). LC-MS (ESI+): 139.0 (M+H⁺).

### Synthesis of N-(Bis-methylsulfanyl-methylene)-3-trifluoromethyl-benzenesulfonamide (I2)

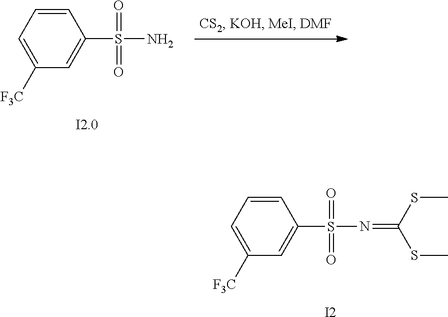 [SMILES: NS(=O)(=O)c1cccc(C(F)(F)F)c1 ; CSC(=NS(=O)(=O)c1cccc(C(F)(F)F)c1)SC ; "I2"]

To a solution of 3-(Trifluoromethyl)benzenesulfonamide (5.0 g, 22.2 mmol, 12.0) and carbon disulfide (2.18 mL, 36.4 mmol, 1.6 eq.) in dimethylformamide (74 mL) cooled in an ice bath was added a solution of Potassium hydroxide (2.94 g, 52.4 mmol, 2.4 eq.) in water (11 mL) dropwise maintaining the internal temperature below 10° C. After stirring 1 hour at 0° C., iodomethane (3.2 mL, 51.5 mmol, 2.3 eq.) was added dropwise while maintaining the internal temperature below 10° C. After stirring up to 2 hours at room temperature, water (35 mL) was added, and the mixture was extracted with ethyl acetate (2×50 mL). The combined organic layers were collected, dried over magnesium sulfate, filtered, and concentrated in vacuo to give the expected product as yellow pale solid without further purification (5.75 g, 17.5 mmol, 79% yield). LC-MS (ESI+): 330.0 (M+H⁺).

### Synthesis of Dimethyl ((2-chlorophenyl)sulfonyl)carbonimidodithioate (I3)

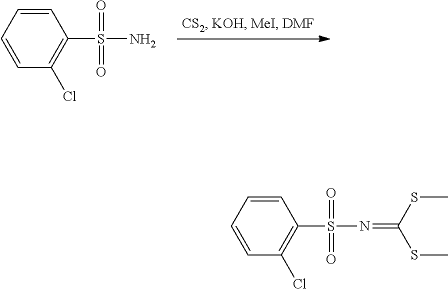 [SMILES: NS(=O)(=O)c1ccccc1Cl ; CSC(=NS(=O)(=O)c1ccccc1Cl)SC]

To a solution of 2-chlorobenzenesulfonamide (4.7 g, 24.5 mmol, 1 eq.) and carbon disulfide (2.4 mL, 40.2 mmol, 1.6 eq.) in dimethylformamide (87 mL) cooled in an ice bath was added a solution of Potassium hydroxide (3.2 g, 57.8 mmol, 2.4 eq.) in water (13 mL) dropwise while maintaining the internal temperature below 10° C. After stirring for 1 hr at 0° C., iodomethane (3.5 mL, 56.9 mmol, 2.3 eq.) was added dropwise while maintaining the internal temperature below 10° C. After warming for 2 hr up to rt, water (35 mL) was added, and the mixture was extracted with ethyl acetate (2×50 mL). The organic layers were collected, dried over magnesium sulfate, filtered, and concentrated in vacuo to give the desired product as yellow pale solid without further purification (6.0 g, 20.3 mmol, 83% yield). LC-MS (ESI+): 295.9 (M+H⁺).

### Synthesis of Dimethyl ((2-ethynylphenyl)sulfonyl)carbonimidodithioate (I4)

### Synthesis of 2-Bromobenzenesulfonamide (I4.1)

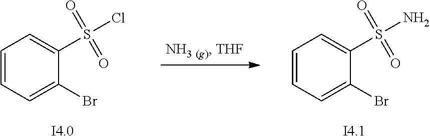 [SMILES: O=S(=O)(Cl)c1ccccc1Br ; NS(=O)(=O)c1ccccc1Br]

Through a solution of 2-bromobenzenesulfonyl chloride (5.0 g, 19.6 mmol, 1.0 eq.) in tetrahydrofuran (100 mL) was bubbled ammonia gas for 10 minutes. Upon completion, the reaction mixture was filtered through a pad of silica, washed with ethyl acetate, and concentrated under reduced pressure to afford a beige solid which was used as such without further purification (4.4 g, 18.8 mmol, 95%). LC-MS (ESI+): 236.0 (M+H⁺).

### Synthesis of 2-((Trimethylsilyl)ethynyl)benzenesulfonamide (I4.2)

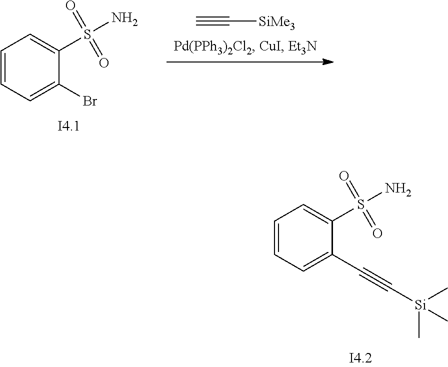

Bis(triphenylphosphine)palladium chloride (0.65 g, 0.93 mmol, 0.05 eq.) and (trimethylsilyl)acetylene (3.3 mL, 23.3 mmol, 1.25 eq.) were added to a solution of 2-bromobenzenesulfonamide (4.4 g, 18.6 mmol, I4.1) and copper (I) iodide (0.18 g, 0.93 mmol, 0.05 eq.) in degassed triethylamine (78 mL). The resulting mixture was refluxed at 90° C. for 18 h. Upon completion, the reaction mixture was filtered through a celite pad, washed with ethyl acetate, concentrated, and purified by flash chromatography eluting on silica with ethyl acetate/heptane mixtures (0/100 to 20/80, v/v) to yield the expected compound as a beige solid (1.8 g, 7.1 mmol, 38% yield). LC-MS (ESI+): 254.0 (M+H⁺).

### Synthesis of Dimethyl ((2-ethynylphenyl)sulfonyl)carbonimidodithioate (I4)

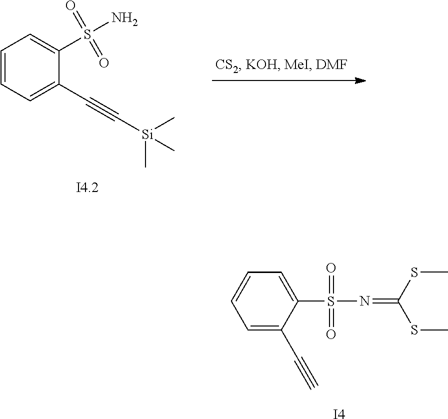

2-((Trimethylsilyl)ethynyl)benzenesulfonamide (1.28 g, 5.05 mmol, 14.2) and carbon disulfide (0.5 mL, 8.3 mmol, 1.6 eq.) were dissolved in dimethylformamide (9 mL) and the mixture was cooled in an ice bath. Then, a solution of Potassium hydroxide (0.67 g, 11.9 mmol, 2.4 eq.) in water (3 mL) was added dropwise while maintaining the internal temperature below 10° C. The resulting mixture was stirred 1 hr at 0° C. Elapsed that time, iodomethane (0.73 mL, 11.7 mmol, 2.3 eq.) was added dropwise while maintaining the internal temperature below 10° C. and the resulting mixture was stirred 2 hr up to rt. Upon consumption of starting material, water (15 mL) was added, and the mixture was extracted with ethyl acetate (2×20 mL). The organic layers were collected, dried over magnesium sulfate, filtered, and concentrated in vacuo. The residue was purified by flash chromatography eluting on silica with ethyl acetate/heptane mixtures (0/100 to 50/50, v/v) affording the expected compound as a yellowish solid (0.47 g, 1.6 mmol, 32% yield). LC-MS (ESI+): 286.0 (M+H⁺).

### Synthesis of Dimethyl ((3-ethynylphenyl)sulfonyl)carbonimidodithioate (I5)

### Synthesis of 3-((Trimethylsilyl)ethynyl)benzenesulfonamide (I5.1)

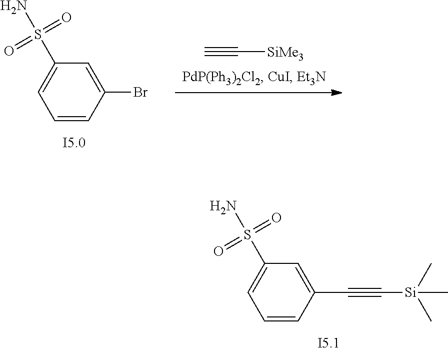

Bis(triphenylphosphine)palladium chloride (0.65 g, 0.93 mmol, 0.05 eq.) and (trimethylsilyl)acetylene (3.3 mL, 23.3 mmol, 1.25 eq.) were added to a solution of 3-bromobenzenesulfonamide (4.4 g, 18.6 mmol, 1.0 eq) and copper (I) iodide (0.18 g, 0.93 mmol, 0.05 eq.) in degassed triethylamine (78 mL). After stirring at 90° C. for 18 h, the reaction mixture was filtered through a celite pad, washed with ethyl acetate, concentrated. The residue was purified by flash chromatography eluting on silica with ethyl acetate/heptane mixtures (0/100 to 20/80, v/v) to yield the expected compound as a beige solid (4.45 g, 17.6 mmol, 94% yield). LC-MS (ESI+): 254.0 (M+H⁺).

### Synthesis of Dimethyl ((3-ethynylphenyl)sulfonyl)carbonimidodithioate (I5)

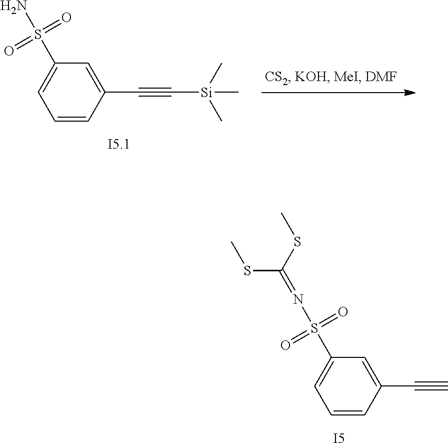

To 3-((Trimethylsilyl)ethynyl)benzenesulfonamide (4.45 g, 17.6 mmol, 15.1) and carbon disulfide (1.7 mL, 28.8 mmol, 1.6 eq.) in dimethylformamide (30 mL) cooled in an ice bath was added a solution of Potassium hydroxide (2.3 g, 41.4 mmol, 2.4 eq.) in water (10 mL) was added dropwise while maintaining the internal temperature below 10° C. After stirring for 1 hr at 0° C., iodomethane (2.5 mL, 40.7 mmol, 2.3 eq.) was added dropwise while maintaining the internal temperature below 10° C. After warming over 2 hr to rt, the reaction was diluted with water (20 mL) and extracted with ethyl acetate (2×30 mL). The organic layers were collected, dried over magnesium sulfate, filtered and concentrated in vacuo. The residue was purified by flash chromatography eluting on silica with ethyl acetate/heptane mixtures (0/100 to 50/50, v/v) to yield the expected compound as a yellowish solid (2.65 g, 9.29 mmol, 53% yield). LC-MS (ESI+): 286.0 (M+H⁺).

### Synthesis of Methyl ((2,4-difluorophenyl)sulfonyl)carbamate (I6)

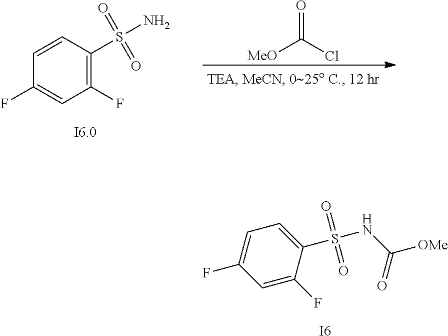

To a solution of 16.0 (0.9 g, 4.66 mmol, CAS #13656-60-5) in acetonitrile (15 mL) at 0° C. were added triethylamine (1.62 mL, 11.65 mmol, 2.5 eq) and methyl carbonochloridate (577.40 μL, 7.45 mmol, 1.6 eq). The resulting mixture was slightly warmed up to rt and stirred at rt for 12 hr. The reaction mixture was quenched with water (25 mL) and concentrated under reduced pressure. The residue was re-dissolved in water (5 mL) and adjusted to pH=3 by aqueous HCl (1N). The suspension was filtered, and the filter cake was dried in high vacuum to give 16 (0.3 g, 1.19 mmol, 26%) as a white solid. ¹H NMR: 400 MHz, DMSO-d₆ δ ppm 3.73 (s, 3H), 6.93-7.13 (m, 2H), 7.92 (br s, 1H), 8.11 (td, J=8.47, 6.05 Hz, 1H)

### Synthesis of I7: methyl ((3,5-difluorophenyl)sulfonyl)carbamate (I7)

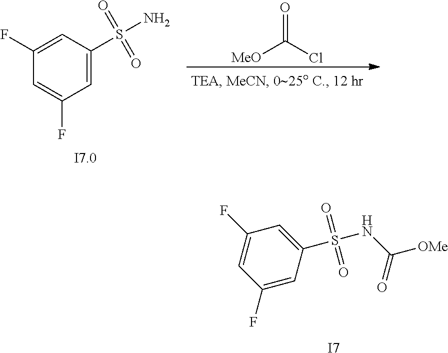

To a solution of 17.0 (5 g, 25.88 mmol, 1 eq) in acetonitrile at 0° C. (50 mL) were added triethylamine (9.01 mL, 64.71 mmol, 2.5 eq) and methyl carbonochloridate (3.21 mL, 41.41 mmol, 1.6 eq). After stirring at rt for 12 hr, the reaction mixture was quenched with water (25 mL) and concentrated under reduced pressure. The residue was re-dissolved in water (5 mL) and adjusted to pH=3 by aqueous HCl (1 N). The suspension was filtered, and the filter cake was dried in high vacuum to give I7 (2 g, 7.56 mmol, 29%) as a white solid. LCMS (ESI+): m/z 239.1 (M+H)⁺; ¹H NMR: 400 MHz, DMSO-d₆δ ppm 3.60 (s, 3H), 7.58 (br d, J=4.77 Hz, 2H), 7.73 (br t, J=9.29 Hz, 1H).

### Synthesis of (R)-2-aminopropane-1-sulfonamide (I8)

### Synthesis of benzyl (R)-(1-hydroxypropan-2-yl)carbamate (I8.1)

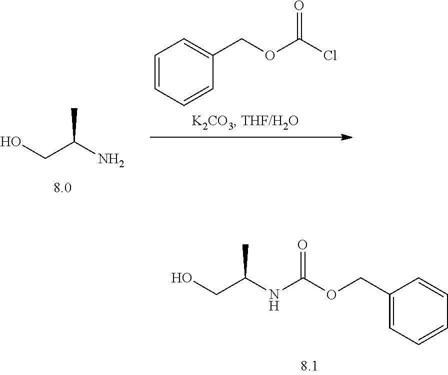

To a solution of D-Alaninol (2.0 g, 26.6 mmol, 18.0) and Potassium carbonate (7.4 g, 53.5 mmol, 2.01 eq.) in tetrahydrofuran/water (50 mL, 1:1) at 0° C. was added benzyl chloroformate (4.2 mL, 29.3 mmol, 1.1 eq.). After stirring at rt for 1 hour, the reaction mixture was extracted with ethyl acetate (75 mL). The organic phase was dried over magnesium sulfate, filtered, and concentrated in vacuo to give a white solid which was used in the next step without further purification (5.5 g, 26.3 mmol, 98% yield). LC-MS (ESI+): 210.1 (M+H⁺).

### Synthesis of (R)-2-(((benzyloxy)carbonyl)amino)propyl methanesulfonate (I8.2)

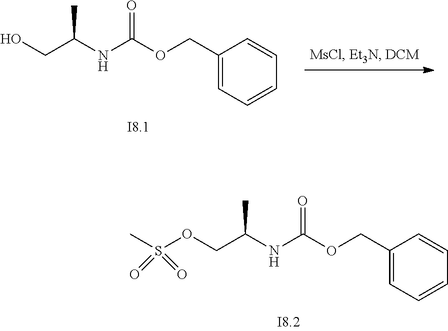 [SMILES: C[C@H](CO)NC(=O)OCc1ccccc1 ; C[C@H](COS(C)(=O)=O)NC(=O)OCc1ccccc1]

Methanesulfonyl chloride (2.12 mL, 27.3 mmol, 1.04 eq.) in dichloromethane (52 mL) was added to a solution of benzyl (R)-(1-hydroxypropan-2-yl)carbamate (5.5 g, 26.3 mmol, 18.1) and triethylamine (4.03 mL, 28.9 mmol, 1.1 eq.) in dichloromethane (104 mL) at 0° C. After stirring for 30 min in ice bath, the mixture was stirred at rt for 30 min more. Upon completion, the reaction mixture was subsequently washed with sat. aqueous sodium hydrogen bicarbonate (80 mL) and brine (80 mL). The organic extract was dried over magnesium sulfate, filtered, and concentrated in vacuo to dryness to afford the expected product as a white solid (7.5 g, 26.2 mmol, 99% yield). LC-MS (ESI+): 288.1 (M+H⁺).

### Synthesis of (R)-S-(2-(((benzyloxy)carbonyl)amino)propyl) ethanethioate (I8.3)

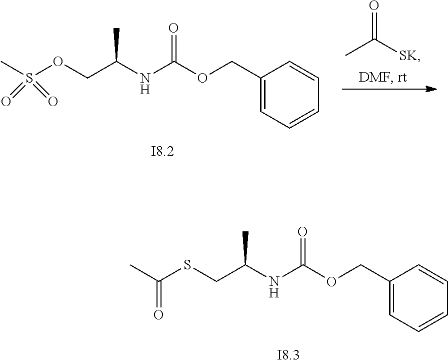

Potassium thioacetate (14.9 g, 130 mmol, 5.0 eq.) was added to a stirring solution of (R)-2-(((benzyloxy)carbonyl)amino)propyl methanesulfonate (7.5 g, 26.0 mmol, 18.2) in dimethylformamide (120 mL). After stirring at rt for 18 hours, solvent was partially removed under reduced pressure, diluted with ethyl acetate (70 mL) and extracted with water (60 mL). The organic phase was washed with brine (50 mL), dried over magnesium sulfate, filtered, and concentrated in vacuo to give a dark oil which was purified by flash chromatography on silica eluting with ethyl acetate/heptane mixtures (0/100 to 50/50, v/v) to give a brownish oil (6.74 g, 25.2 mmol, 96% yield). LC-MS (ESI+): 268.1 (M+H⁺).

### Synthesis of benzyl (R)-(1-(chlorosulfonyl)propan-2-yl)carbamate (I8.4)

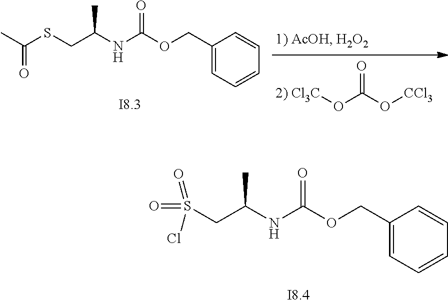

A solution of hydrogen peroxide 50% (15 mL, 0.63 mL/mmol) in acetic acid (55 mL) was added dropwise to a solution of (R)-S-(2-(((benzyloxy)carbonyl)amino)propyl) ethanethioate (6.74 g, 25.2 mmol, 18.3) in acetic acid (40 mL). After stirring at rt for 18 hours, palladium on activated carbon was added to destroy excess peroxide and the resulting mixture was filtered through celite pad. The filtrate was concentrated in vacuo to dryness.

The brown oil was taken in dichloromethane (120 mL) and triphosgene (10.4 g, 35.3 mmol, 1.4 eq.) followed up by dimethylformamide (3.5 mL). After stirring at rt for 18 hours, solvents were removed under reduced pressure. The residue was chromatographed on silica eluting with dichloromethane/methanol mixtures (0/100 to 10/90, v/v) to give a beige solid (5.9 g, 20.3 mmol, 80% yield). LC-MS (ESI+): 292.0 (M+H+).

### Synthesis of benzyl (R)-(1-sulfamoylpropan-2-yl)carbamate (I8.5)

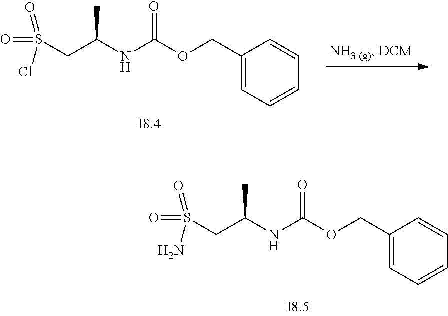 [SMILES: C[C@H](CS(=O)(=O)Cl)NC(=O)OCc1ccccc1 ; C[C@H](CS(N)(=O)=O)NC(=O)OCc1ccccc1]

Ammonia gas was bubbled through a stirred solution of benzyl (R)-(1-(chlorosulfonyl)propan-2-yl)carbamate (5.9 g, 20.2 mmol, 18.4) in dichloromethane (200 mL) at rt for 15 min. Upon completion, the reaction mixture was concentrated in vacuo to dryness and the white pale solid obtained was used in the next step without further purification (5.48 g, 20.1 mmol, 99% yield). LC-MS (ESI+): 273.0 (M+H+).

### Synthesis of (R)-2-aminopropane-1-sulfonamide (I8)

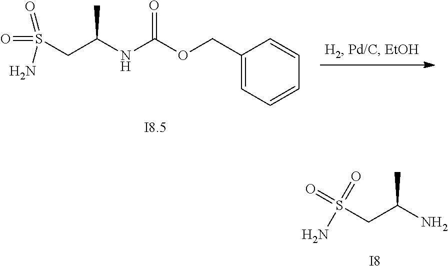

To a solution of benzyl (R)-(1-sulfamoylpropan-2-yl)carbamate (5.48 g, 20.2 mmol, 18.5) in ethanol (200 mL) was added Palladium 10% on carbon powder (Pd/C, 10 wt. %) (2.0 g, 0.1 eq.). The mixture was degassed in vacuo and then saturated with hydrogen, repeated 3 times. The reaction mixture was stirred at rt under hydrogen atmosphere for 16 hours. Upon completion, the mixture was filtered through celite pad and concentrated in vacuo to afford the expected compound as a brown solid (2.7 g, 19.6 mmol, 97% yield). LC-MS (ESI+): 139.0 (M+H⁺).

### Synthesis of Dimethyl ((4-ethynylphenyl)sulfonyl)carbonimidodithioate (I9)

### Synthesis of ((Trimethylsily)ethynyl)benzenesulfonamide (I9.1)

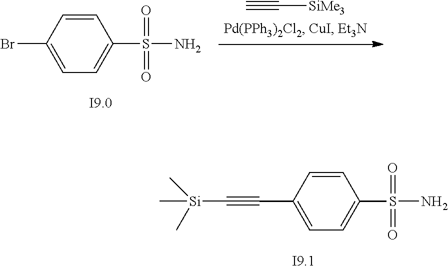

Bis(triphenylphosphine)palladium chloride (0.45 g, 0.63 mmol, 0.05 eq.) and (trimethylsilyl)acetylene (2.26 mL, 15.9 mmol, 1.25 eq.) were added to a solution of 4-bromobenzenesulfonamide (3.0 g, 12.7 mmol, 19.0) and copper (I) iodide (0.12 g, 0.63 mmol, 0.05 eq.) in triethylamine (48 mL). After stirring at 90° C. for 18 h, the reaction mixture was filtered through celite pad, washed with ethyl acetate and concentrated to dryness. The residue was purified by flash chromatography eluting on silica with ethyl acetate/heptane mixtures (0/100 to 20/80, v/v) to afford a beige solid (2.8 g, 11.1 mmol, 87% yield). LC-MS (ESI+): 254.1 (M+H⁺).

### Synthesis of Dimethyl ((4-ethynylphenyl)sulfonyl)carbonimidodithioate (I9)

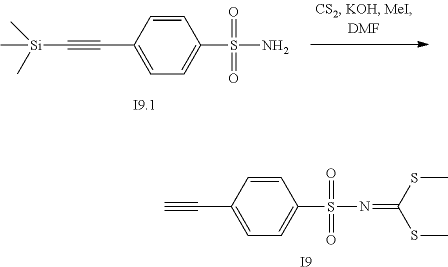

4-((Trimethylsilyl)ethynyl)benzenesulfonamide (0.8 g, 3.16 mmol, 19.1) and carbon disulfide (0.31 mL, 5.18 mmol, 1.6 eq.) dissolved in DMF (4.5 mL) in an ice bath was added a solution of Potassium hydroxide (0.42 g, 7.4 mmol, 2.4 eq.) in water (1.5 mL) was added dropwise while maintaining the internal temperature below 10° C. After stirring for 1 hour at 0° C., iodomethane (0.46 mL, 7.3 mmol, 2.3 eq.) was added dropwise while maintaining the internal temperature below 10° C. After stirring 2 hours up to rt, the reaction was diluted with water (15 mL) and extracted with ethyl acetate (2×20 mL). The organic layers were collected, dried over magnesium sulfate, filtered, and concentrated in vacuo. The residue was purified by flash chromatography eluting on silica with ethyl acetate/heptane mixtures (0/100 to 50/50, v/v). The desired fractions were collected and concentrated in vacuo to afford the expected compound as an orange solid (0.39 g, 1.37 mmol, 43% yield). LC-MS (ESI+): 286.0 (M+H⁺).

### Synthesis of (S)-2-amino-3-methylbutane-1-sulfonamide (I10)

### Synthesis of(S)-2-(1-hydroxy-3-methylbutan-2-yl)isoindoline-1,3-dione (I10.1)

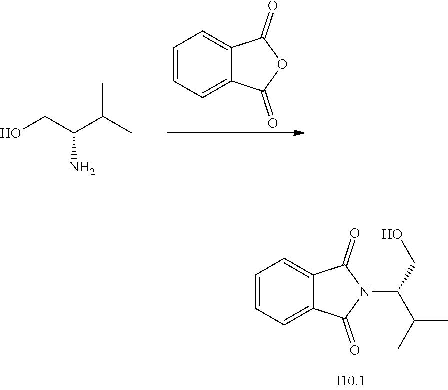

(S)-Valinol (5.00 g, 48.4 mmol, 1 eq.) and phthalic anhydride (7.54 g, 50.9 mmol, 1.05 eq.) were fused at 140° C. in a closed cap flask with stirring for 18 h. Upon completion, the reaction mixture was diluted with ethyl acetate (75 mL) then sequentially washed with sat. aqueous sodium bicarbonate (60 mL), water (50 mL), 10% aq. citric acid (50 mL) and brine (50 mL). The organic extract was dried over magnesium sulfate, filtered and concentrated in vacuo to give an oily crude which was purified by column chromatography on silica eluting with heptane/ethyl acetate mixtures to yield (S)-2-(1-hydroxy-3-methylbutan-2-yl)isoindoline-1,3-dione as a yellowish oil (10.2 g, 43.8 mmol, 90% yield). LC-MS (ESI+): 234.112 (M+H⁺).

### Synthesis of(S)-S-(2-(1,3-dioxoisoindolin-2-yl)-3-methylbutyl) ethanethioate (I10.2)

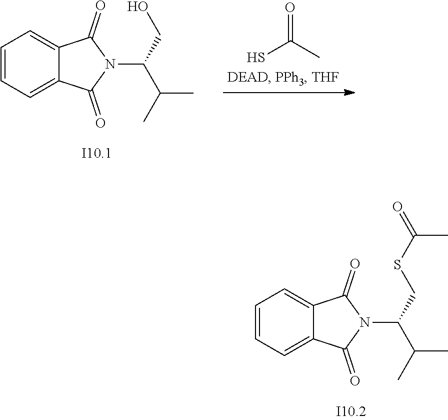

To a solution of diethyl azodicarboxylate in toluene (40%, 2.15 mL, 4.71 mmol, 2.2 eq.) and triphenylphosphine (1.24 g, 4.71 mmol, 2.2 eq) in THE (10 mL) at rt was added sequentially (S)-2-(1-hydroxy-3-methylbutan-2-yl)isoindoline-1,3-dione (0.5 g, 2.14 mmol, 110.1) dissolved in THE (10 mL) and thioacetic acid (0.34 mL, 4.71 mmol, 2.2 eq.). After stirring at rt for 18 hours, the mixture was diluted with ethyl acetate (60 mL) and washed with aqueous sodium carbonate solution (1 M, 50 mL). The organic extract was dried over magnesium sulfate, filtered, and concentrated in vacuo. The residue was purified by flash chromatography on silica eluting with heptane/ethyl acetate mixtures (100/0 to 50/50, v/v) to yield (S)-S-(2-(1,3-dioxoisoindolin-2-yl)-3-methylbutyl) ethanethioate (110.2) as a yellow oil (0.51 g, 1.75 mmol, 82% yield). LC-MS (ESI+): 292.1 (M+H⁺).

### Synthesis of (S)-2-(1,3-dioxoisoindolin-2-yl)-3-methylbutane-1-sulfonic acid (I10.3)

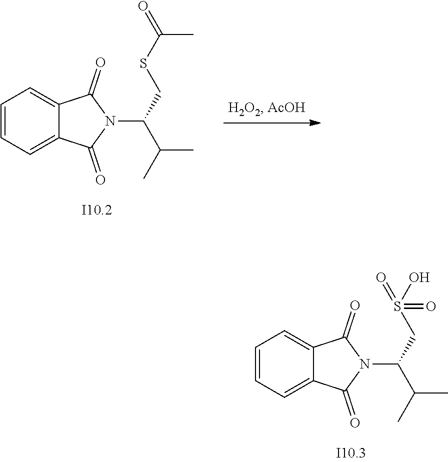 [SMILES: CC(=O)SC[C@H](C(C)C)N1C(=O)c2ccccc2C1=O ; CC(C)[C@@H](CS(=O)(=O)O)N1C(=O)c2ccccc2C1=O]

A mixture of hydrogen peroxide and acetic acid (1:2, v/v) (9.30 mL) was added to a solution of (S)-S-(2-(1,3-dioxoisoindolin-2-yl)-3-methylbutyl) ethanethioate (1.03 g, 3.53 mmol, I10.2) in acetic acid (3 mL). After stirring at rt for 18 hours, palladium on activated carbon was added to destroy excess peroxide. The resulting mixture was filtered through celite pad and the filtrate was concentrated in vacuo. The oily residue was co-evaporated with toluene (2×10 mL) and concentrated to dryness affording a brown oil which was used in the next step without further purification (0.99 g, 3.33 mmol, 94% yield). LC-MS (ESI+): 298.074 (M+H⁺).

### Synthesis of(S)-2-(1,3-dioxoisoindolin-2-yl)-3-methylbutane-1-sulfonyl chloride (I10.4)

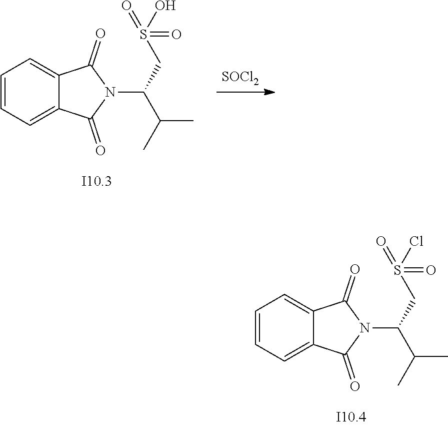

(S)-2-(1,3-dioxoisoindolin-2-yl)-3-methylbutane-1-sulfonic acid (0.94 g, 3.17 mmol, 110.3) was refluxed in thionyl chloride (3 mL) at 80° C. for 16 hours. Upon completion, the reaction mixture was concentrated and co-evaporate 3 times with toluene to give a brown crude which was purified by flash chromatography eluting with heptane/ethyl acetate mixtures (100/0 to 50/50) to yield (S)-2-(1,3-dioxoisoindolin-2-yl)-3-methylbutane-1-sulfonyl chloride I10.4 as a yellowish thick oil (0.55 g, 1.75 mmol, 55% yield). LC-MS (ESI+): 316.040 (M+H⁺).

### Synthesis of(S)-2-(1,3-dioxoisoindolin-2-yl)-3-methylbutane-1-sulfonamide (I10.5)

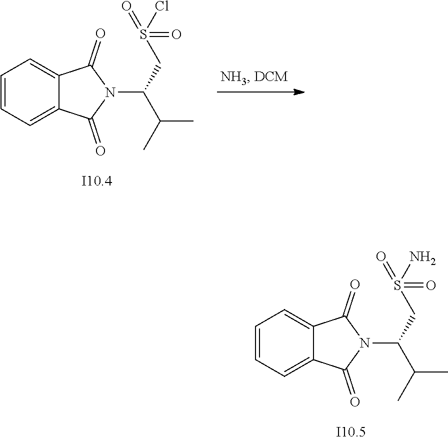

Ammonia gas was bubbled through a stirred solution of (S)-2-(1,3-dioxoisoindolin-2-yl)-3-methylbutane-1-sulfonyl chloride (0.51 g, 1.62 mmol, 110.4) in dichloromethane (15 mL) at rt until no more precipitate formed. Upon completion, the reaction mixture was concentrated in vacuo to dryness and the white solid obtained was used in the next step without further purification (0.45 g, 1.52 mmol, 94% yield). LC-MS (ESI+): 297.090 (M+H⁺).

### Synthesis of(S)-2-amino-3-methylbutane-1-sulfonamide (I10)

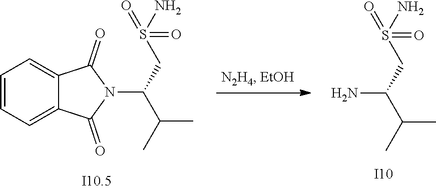

Hydrazine hydrate (0.32 mL, 4.55 mmol, 3 eq.) was added to a solution of (S)-2-(1,3-dioxoisoindolin-2-yl)-3-methylbutane-1-sulfonamide (0.45 g, 1.52 mmol, 110.5) in ethanol (8.5 mL). After stirring at reflux for 5 h, the reaction mixture was filtered and washed with ethanol. The filtrate was concentrated in vacuo to dryness affording (S)-2-amino-3-methylbutane-1-sulfonamide I10.6 as a white solid which was used as such without further purification (0.25 g, 1.51 mmol, 99% yield). LC-MS (ESI+): 167.100 (M+H⁺).

### Synthesis of Dimethyl ((1-methyl-1H-imidazol-4-yl)sulfonyl)carbonimidodithioate (I11)

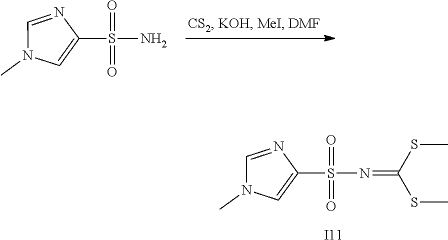

To 1-Methyl-1H-imidazole-4-sulfonamide (0.47 g, 2.9 mmol, 1 eq.) and carbon disulfide (0.29 mL, 4.8 mmol, 1.6 eq.) dissolved in DMF (10 mL) cooled in an ice bath was added a solution of Potassium hydroxide (0.39 g, 6.9 mmol, 2.4 eq.) in water (2 mL) dropwise while maintaining the internal temperature below 10° C. After stirring for 1 hour at 0° C., iodomethane (0.42 mL, 6.8 mmol, 2.3 eq.) was added dropwise while maintaining the internal temperature below 10° C. After stirring for 1 hour up to room temperature, the reaction was diluted with water (15 mL) was added extracted with ethyl acetate (30 mL×2). The organic layers were collected, dried over magnesium sulfate, filtered, and concentrated in vacuo. The residue was purified by flash chromatography eluting on silica with methanol/dichloromethane mixtures (0/100 to 10/90, v/v). The desired fractions were collected and concentrated in vacuo to afford the expected compound as a white solid (65 mg, 0.25 mmol, 8% yield). LC-MS (ESI+): 265.9 (M+H⁺).

### Synthesis of Dimethyl ((2-isopropyl-2H-1,2,3-triazol-4-yl)sulfonyl)carbonimidodithioate (I12)

### Synthesis of 4-(Benzylthio)-1H-1,2,3-triazole (I12.1)

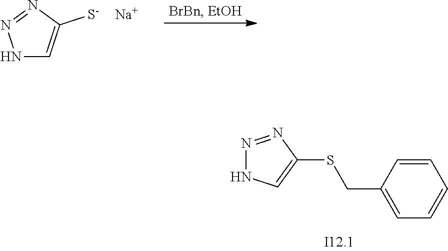 [SMILES: [S-]c1c[nH]nn1 ; [Na+] ; c1ccc(CSc2c[nH]nn2)cc1]

To a solution of sodium 1H-1,2,3-triazole-4-thiolate (5.0 g, 40.61 mmol, 1.0 eq.) in ethanol (50 mL) at 0° C. was added dropwise benzyl bromide (4.83 mL, 40.6 mmol, 1.0 eq.) over 5 min. After warming to room temperature and stirring for 1 hour, solvent was removed under reduced pressure. The residue was diluted with sodium hydrogen carbonate saturated solution (40 mL) and extracted with ethyl acetate (50 mL). The organic phase was washed with brine (40 mL), dried over magnesium sulfate, filtered, and concentrated in vacuo to give the expected compound as a white solid (7.7 g, 40.31 mmol, 99%.). LC-MS (ESI+): 192.1 (M+H⁺).

### Synthesis of Intermediate 4-(benzylthio)-2-isopropyl-2H-1,2,3-triazole (I12.2 to I12.4)

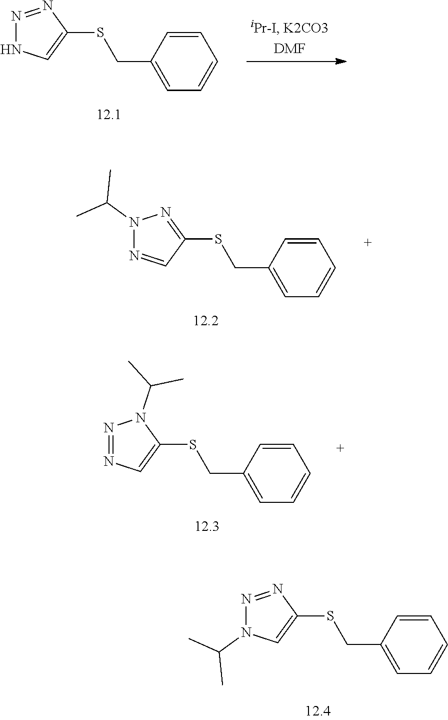 [SMILES: c1ccc(CSc2c[nH]nn2)cc1 ; CC(C)n1ncc(SCc2ccccc2)n1 ; CC(C)n1nncc1SCc1ccccc1 ; CC(C)n1cc(SCc2ccccc2)nn1]

To a solution of 4-(benzylthio)-1H-1,2,3-triazole (7.7 g, 40.2 mmol, 12.1) in dimethylformamide (80 mL) at 0° C. with Potassium carbonate (14.0 g, 100.6 mmol, 2.5 eq.) was added 2-iodopropane (8.05 mL, 80.5 mmol, 2.0 eq.) dropwise. After warming to rt and stirring for 18 hours more, solvent was partially removed. The mixture was diluted with water (40 mL) and extracted with ethyl acetate (2×50 mL). The organic phases were collected, dried over magnesium sulfate, filtered, and concentrated in vacuo to give an oil which was purified by flash chromatography eluting with ethyl acetate/heptane mixtures (0/100 to 20/80, v/v) to afford the corresponding triazole regioisomers as colorless oils.

4-(benzylthio)-2-isopropyl-2H-1,2,3-triazole (I12.2)-5.36 g, 57%. LC-MS (ESI+): 234.1 (M+H⁺); 1H NMR (400 MHz, DMSO-d₆, 25° C.): δ=7.65 (s, 1H), 7.19-7.29 (m, 5H), 4.69-4.81 (m, 1H), 4.16 (s, 2H), 1.43 ppm (d, J=6.7 Hz, 6H).

5-(benzylthio)-1-isopropyl-1H-1,2,3-triazole (I12.3)-1.04 g, 11%. LC-MS (ESI+): 234.1 (M+H+); 1H NMR (400 MHz, DMSO-d₆, 25° C.): δ=7.76 (s, 1H), 7.21-7.30 (m, 3H), 7.17 (dd, J=7.7, 1.4 Hz, 2H), 4.59 (spt, J=6.7 Hz, 1H), 4.10 (s, 2H), 1.28 ppm (d, J=6.8 Hz, 6H).

4-(benzylthio)-1-isopropyl-1H-1,2,3-triazole (I12.4)-1.95 g, 21%. LC-MS (ESI+): 234.1 (M+H⁺); 1H NMR (400 MHz, DMSO-d₆, 25° C.): δ=8.07 (s, 1H), 7.18-7.28 (m, 5H), 4.75 (spt, J=6.7 Hz, 1H), 4.09-4.12 (m, 2H), 1.43 ppm (d, J=6.8 Hz, 6H).

### Synthesis of 2-Isopropyl-2H-1,2,3-triazole-4-sulfonyl chloride (I12.5)

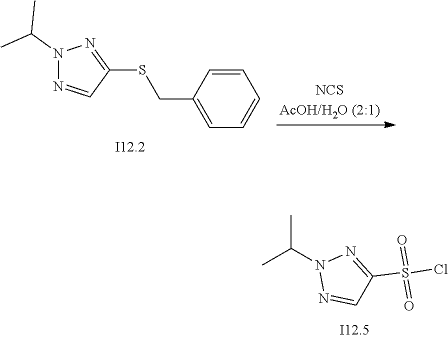 [SMILES: CC(C)n1ncc(SCc2ccccc2)n1 ; CC(C)n1ncc(S(=O)(=O)Cl)n1]

To a solution of 4-(benzylthio)-2-isopropyl-2H-1,2,3-triazole (5.36 g, 23.0 mmol, I12.5) in a mixture of AcOH/H2O (46/23 mL) at 0° C. was added N-chlorosuccinimide (12.3 g, 91.9 mmol, 4.0 eq.). After stirring at room temperature for 18 h, the reaction mixture was diluted with water (20 mL) and extracted with ethyl acetate (75 mL). The organic phase was subsequently washed with sat. sodium hydrogen carbonate solution (50 mL) and brine (50 mL), dried over magnesium sulfate, filtered, and concentrated in vacuo. The residue was purified by flash chromatography eluting with ethyl acetate/heptane mixtures (0/100 to 20/80, v/v) to afford the expected sulfonyl chloride as a colorless oil (4.1 g, 19.6 mmol, 85%.). LC-MS (ESI+): 210.0 (M+H⁺).

### Synthesis of 2-isopropyl-2H-1,2,3-triazole-4-sulfonamide (I12.6)

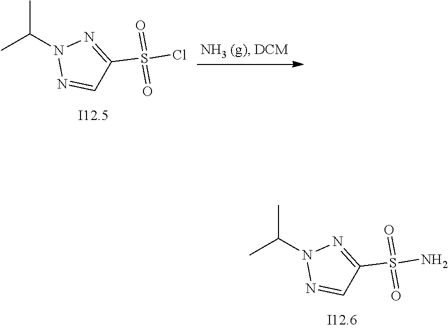

Ammonia gas was bubbled through a solution of 2-isopropyl-2H-1,2,3-triazole-4-sulfonyl chloride (4.70 g, 22.4 mmol, 112.5) in dichloromethane (100 mL) at room temperature for 10 minutes. Upon completion, solvent was removed under reduced pressure and the resulting slurry was triturated with ethyl acetate. The precipitates were removed by filtration through a celite pad, and the filtrate was concentrated in vacuo to afford the sulfonamide as a yellow oil which was used in the next step without further purification (4.2 g, 22.1 mmol, 98% yield). LC-MS (ESI+): 191.1 (M+H⁺).

### Synthesis of Dimethyl ((2-isopropyl-2H-1,2,3-triazol-4-yl)sulfonyl)carbonimidodithioate (I12)

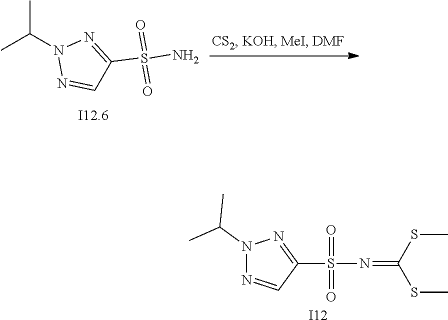

2-Isopropyl-2H-1,2,3-triazole-4-sulfonamide (4.2 g, 22.1 mmol, I12.6) and carbon disulfide (2.2 mL, 36.2 mmol, 1.6 eq.) in dimethylformamide (36 mL) cooled in an ice bath was added a solution of Potassium hydroxide (2.9 g, 52.1 mmol, 2.4 eq.) in water (12 mL) dropwise while maintaining the internal temperature below 10° C. After stirring 1 hour at 0° C., iodomethane (3.2 mL, 51.2 mmol, 2.3 eq.) was added dropwise while maintaining the internal temperature below 10° C. After stirring for 2 hours up to room temperature, water (20 mL) was added and the mixture was extracted with ethyl acetate (2×50 mL). The organic layers were collected, dried over magnesium sulfate, filtered, and concentrated in vacuo. The residue was purified by flash chromatography using ethyl acetate/heptane mixtures (0/100 to 30/70, v/v), affording the expected compound as a white solid (2.9 g, 9.86 mmol, 45% yield). LC-MS (ESI+): 295.0 (M+H⁺).

### Synthesis of Dimethyl ((2-methyl-2H-1,2,3-triazol-4-yl)sulfonyl)carbonimidodithioate (I13)

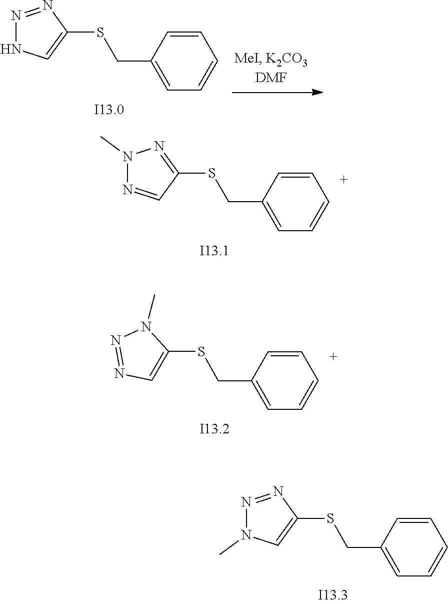

### Synthesis of I13.1, I13.2 and I13.3

To a solution of 4-(benzylthio)-1H-1,2,3-triazole (7.75 g, 40.5 mmol, I13.0) in dimethylformamide (80 mL) at 0° C. with Potassium carbonate (12.3 g, 89.1 mmol, 2.2 eq.) was added 2-iodomethane (5.04 mL, 81.0 mmol, 2.0 eq.) dropwise to the mixture at 0° C. After stirring at room temperature for 18 hours, solvent was partially removed. The mixture was diluted with water (40 mL) and extracted with ethyl acetate (2×50 mL). The organic phases were collected, dried over magnesium sulfate, filtered, and concentrated in vacuo to give an oil which was purified by flash chromatography eluting with ethyl acetate/heptane mixtures (0/100 to 20/80, v/v) to afford the corresponding triazole regioisomers as colorless oils.

4-(Benzylthio)-2-methyl-2H-1,2,3-triazole (I13.1)-3.02 g, 36%. ¹H NMR (400 MHz, DMSO-d₆, 25° C.): δ=7.67 (s, 1H), 7.2-7.3 (m, 5H), 4.18 (s, 2H), 4.10 (s, 3H) ppm. LC-MS (ESI+): 206.1 (M+H⁺); HPLC RT: 1.529 min (Method: TACC21-6).

5-(benzylthio)-1-methyl-1H-1,2,3-triazole (I13.2)-1.04 g, 11%. ¹H NMR (400 MHz, DMSO-d₆, 25° C.): δ=7.70 (s, 1H), 7.2-7.3 (m, 3H), 7.1-7.2 (m, 2H), 4.09 (s, 2H), 3.73 (s, 3H) ppm. LC-MS (ESI+): 206.1 (M+H⁺); HPLC RT: 1.334 min (Method: TACC21-6).

4-(benzylthio)-1-methyl-1H-1,2,3-triazole (I13.3)-1.95 g, 21%. ¹H NMR (400 MHz, DMSO-d₆, 25° C.): δ=8.01 (s, 1H), 7.2-7.3 (m, 5H), 4.11 (s, 2H), 3.99 (s, 3H) ppm. LC-MS (ESI+): 206.1 (M+H⁺); HPLC RT: 1.250 min (Method: TACC21-6).

### Synthesis of 2-Methyl-2H-1,2,3-triazole-4-sulfonyl chloride (I13.4)

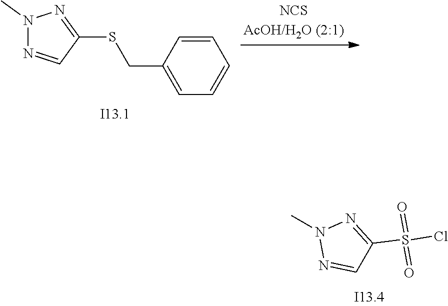

To a solution of 4-(benzylthio)-2-methyl-2H-1,2,3-triazole (3.0 g, 14.7 mmol, 113.1) in a mixture of acetic acid (46 mL) and water (23 mL) at 0° C. was added N-Chlorosuccinimide (7.8 g, 58.8 mmol, 4.0 eq.). After stirring at room temperature for 18 h, the reaction mixture was diluted with water (20 mL) and extracted with ethyl acetate (75 mL). The organic phase was subsequently washed with sat. sodium hydrogen carbonate solution (50 mL) and brine (50 mL), dried over magnesium sulfate, filtered, and concentrated in vacuo to afford the expected sulfonyl chloride as a colorless oil which was used without further purification (2.5 g, 13.8 mmol, 95%). LC-MS (ESI+): 182.1 (M+H⁺).

### Synthesis of 2-Methyl-2H-1,2,3-triazole-4-sulfonamide (I13.5)

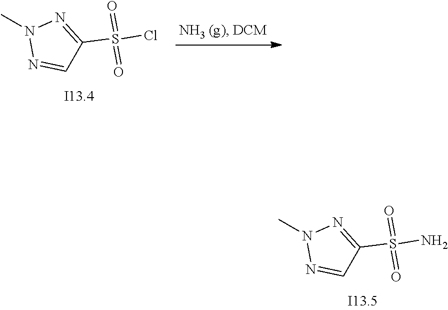

Ammonia gas was bubbled through a solution of 2-methyl-2H-1,2,3-triazole-4-sulfonyl chloride (2.56 g, 14.1 mmol, 113.4) in dichloromethane (100 mL) at room temperature for 10 minutes. Upon completion, solvent was removed under reduced pressure, and the resulting slurry was triturated with ethyl acetate. The precipitates were removed by filtration through a celite pad, and the filtrate was concentrated in vacuo to afford the sulfonamide as a yellow oil which was used in the next step without further purification (2.2 g, 13.6 mmol, 97% yield). LC-MS (ESI+): 163.0 (M+H⁺).

### Dimethyl ((2-methyl-2H-1,2,3-triazol-4-yl)sulfonyl)carbonimidodithioate (I13)

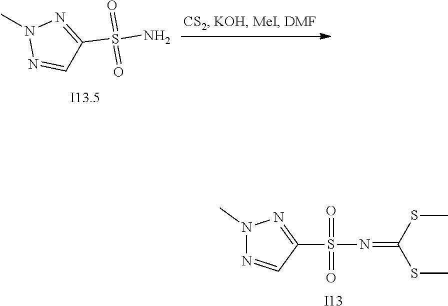

To 2-Methyl-2H-1,2,3-triazole-4-sulfonamide (2.2 g, 13.7 mmol, I13.5) and carbon disulfide (1.35 mL, 22.4 mmol, 1.6 eq.) in dimethylformamide (21 mL) cooled in an ice bath was added a solution of Potassium hydroxide (1.8 g, 32.3 mmol, 2.4 eq.) in water (7 mL) dropwise while maintaining the internal temperature below 10° C. After stirring for 1 hour at 0° C., iodomethane (2.0 mL, 31.8 mmol, 2.3 eq.) was added dropwise while maintaining the internal temperature below 10° C. After stirring for 2 hours up to room temperature, the mixture was diluted with water (20 mL) and extracted with ethyl acetate (2×50 mL). The organic layers were collected, dried over magnesium sulfate, filtered, and concentrated in vacuo. The residue was purified by flash chromatography using ethyl acetate/heptane mixtures (0/100 to 30/70, v/v), affording the expected compound as a yellowish viscous oil (1.17 g, 4.4 mmol, 32% yield). LC-MS (ESI+): 267.0 (M+H⁺).

### Synthesis of Intermediate Dimethyl ((1-isopropyl-1H-1,2,3-triazol-5-yl)sulfonyl)-carbonimidodithioate (I14)

### Synthesis of (E)-3-(4-chlorophenyl)-N′-((2-isopropyl-2H-1,2,3-triazol-4-yl)sulfonyl)-4-phenyl-N-(2-sulfamoylethyl)-4,5-dihydro-1H-pyrazole-1-carboximidamide (I14.1)

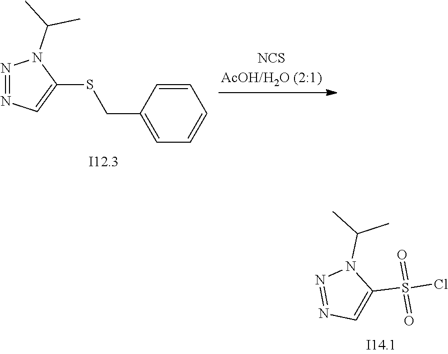 [SMILES: CC(C)n1nncc1SCc1ccccc1 ; CC(C)n1nncc1S(=O)(=O)Cl]

To a solution of 5-(benzylthio)-1-isopropyl-1H-1,2,3-triazole (1.04 g, 4.5 mmol, I12.3) in acetic acid (8 mL) and water (4 mL) at 0° C. was added N-chlorosuccinimide (2.4 g, 17.8 mmol, 4.0 eq.). After stirring at room temperature for 18 h, the reaction mixture was diluted with water (10 mL) and extracted with ethyl acetate (30 mL). The organic phase was washed with sat. sodium hydrogen carbonate solution (25 mL) and brine (25 mL), dried over magnesium sulfate, filtered, and concentrated in vacuo. The residue was purified by flash chromatography eluting with ethyl acetate/heptane mixtures (0/100 to 20/80, v/v) to afford the expected sulfonyl chloride as a white solid (0.62 g, 2.9 mmol, 67%.). LC-MS (ESI+): 210.0 (M+H⁺).

### Synthesis of 1-Isopropyl-1H-1,2,3-triazole-5-sulfonamide (I14.2)

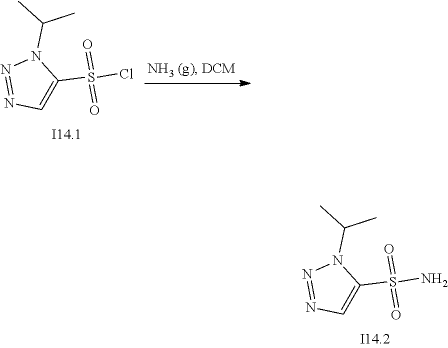

Ammonia gas was bubbled through a solution of 1-isopropyl-1H-1,2,3-triazole-5-sulfonyl chloride (0.62 g, 2.97 mmol, I14.1) in dichloromethane (30 mL) at room temperature for 10 minutes. Upon completion, solvent was removed under reduced pressure and the resulting slurry was triturated with ethyl acetate. The precipitates were removed by filtration through a celite pad, and the filtrate was concentrated in vacuo to afford the sulfonamide as a yellow oil which was used in the next step without further purification (0.55 g, 2.89 mmol, 97% yield). LC-MS (ESI+): 191.0 (M+H⁺).

### Synthesis of Dimethyl ((1-isopropyl-1H-1,2,3-triazol-5-yl)sulfonyl)carbonimidodithioate (I14)

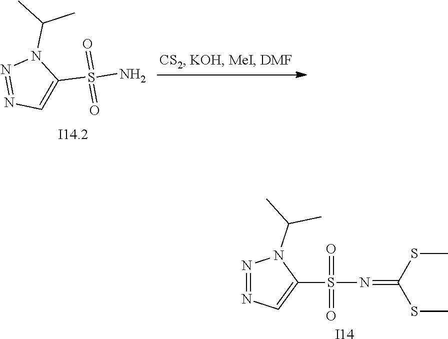

To 1-Isopropyl-1H-1,2,3-triazole-5-sulfonamide (0.55 g, 2.9 mmol, 114.2) and carbon disulfide (0.28 mL, 4.7 mmol, 1.6 eq.) in dimethylformamide (6 mL) cooled in an ice bath was added a solution of Potassium hydroxide (0.4 g, 6.8 mmol, 2.4 eq.) in water (2 mL) dropwise while maintaining the internal temperature below 10° C. After stirring 1 hour at 0° C., iodomethane (0.42 mL, 6.7 mmol, 2.3 eq.) was added dropwise while maintaining the internal temperature below 10° C. After stirring for 2 hours up to room temperature, the mixture was diluted with water (10 mL) and extracted with ethyl acetate (2×25 mL). The organic layers were collected, dried over magnesium sulfate, filtered, and concentrated in vacuo. The residue was purified by flash chromatography using ethyl acetate/heptane mixtures (0/100 to 30/70, v/v), affording the expected compound as a colorless oil (0.29 g, 0.97 mmol, 34% yield). LC-MS (ESI+): 295.0 (M+H⁺).

### Synthesis of Methyl ((1-methyl-1H-pyrazol-4-yl)sulfonyl)carbamate (I15)

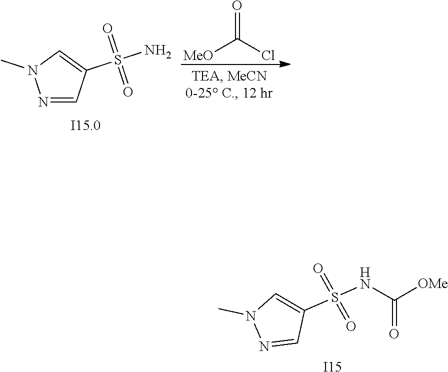

To a solution of 115.0 (300 mg, 1.86 mmol, 1 eq) in acetonitrile (3 mL) at 0° C. were added triethylamine (647.65 μL, 4.65 mmol, 2.5 eq) and methyl carbonochloridate (230.67 μL, 2.98 mmol, 1.6 eq). After stirring at rt for 12 hr, the reaction mixture was quenched by water (5 mL) and concentrated under reduced pressure to give a residue. The residue was re-dissolved in water (5 mL). The mixture was adjusted to pH=3 by aqueous HCl (1 N) and filtered. The filter cake was dried in high vacuum to give I15 (300 mg) as a white solid, which was used for the next step without further purification. LCMS (ESI+): m/z 219.9 (M+H)⁺.

### Synthesis of 3-Amino-2-methylpropane-1-sulfonamide (I16)

### Synthesis of Benzyl (3-hydroxy-2-methylpropyl)carbamate (16.1)

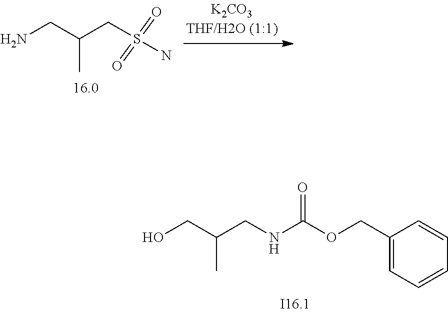

To a solution of 3-amino-2-methyl-propan-1-ol (3.67 g, 41.2 mmol, 1.0 eq.) and Potassium carbonate (11.4 g, 82.3 mmol, 2.0 eq.) in tetrahydrofuran/water (1:1) (136 mL) was added benzyl chloroformate (6.4 mL, 45.3 mmol, 1.1 eq.) at 0° C. After stirring at rt for 1 h, solvents were partially removed under reduced pressure. The mixture was extracted with ethyl acetate (60 mL), separated, dried over magnesium sulfate, filtered, concentrated in vacuo. The residue was purified by flash chromatography on silica eluting with ethyl acetate/heptane (0/100 to 100/0, v/v). The desired fractions were collected and concentrated in vacuo to afford the expected compound as a colorless oil (0.6 g, 6% yield). LC-MS (ESI+): 224.1 (M+H⁺).

### Synthesis of 3-(((Benzyloxy)carbonyl)amino)-2-methylpropyl methanesulfonate (I16.2)

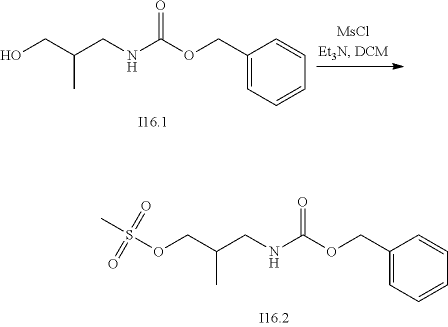 [SMILES: CC(CO)CNC(=O)OCc1ccccc1 ; CC(CNC(=O)OCc1ccccc1)COS(C)(=O)=O]

To a mixture of benzyl (3-hydroxy-2-methylpropyl)carbamate (0.562 g, 2.517 mmol, I16.1) and triethylamine (0.39 mL, 2.77 mmol, 1.1 eq.) in dichloromethane (8 mL) was added methane sulfonyl chloride (0.2 mL, 2.6 mmol, 1.05 eq.) at 0° C. After stirring at rt for 1 h, the reaction was diluted with dichloromethane (25 mL) and washed with ammonium chloride (20 mL). The organic layer was separated, dried over magnesium sulfate, filtered, and concentrated in vacuo to give the expected product as a colorless oil (0.73 g, 97% yield). LC-MS (ESI+): 302.7 (M+H⁺).

### Synthesis of S-(3-(((benzyloxy)carbonyl)amino)-2-methylpropyl) ethanethioate (I16.3)

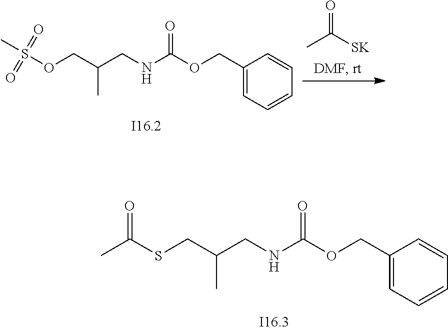

Potassium thioacetate (1.4 g, 12.2 mmol, 5.0 eq.) was added to a solution of 3-(((benzyloxy)carbonyl)amino)-2-methylpropyl methanesulfonate (0.73 g, 2.4 mmol, 116.2) in dimethylformamide (9 mL). After stirring at rt for 18 h, the reaction mixture was diluted with ethyl acetate (45 mL) and washed with water (30 mL) and brine (30 mL). The organic phase was dried over magnesium sulfate, filtered, and concentrated in vacuo. The residue was chromatographed on silica eluting with ethyl acetate/heptane mixtures (0/100 to 70/30, v/v). The desired fractions were collected and concentrated under reduced pressure to afford the expected compound as a yellow oil (0.49 g, 71% yield). LC-MS (ESI+): 281.9 (M+H⁺).

### Synthesis of Benzyl (2-methyl-3-sulfamoylpropyl)carbamate (I16.4)

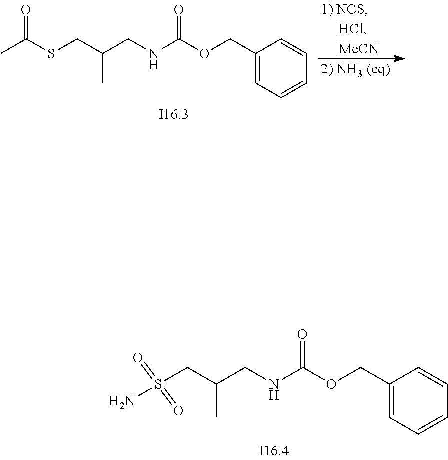 [SMILES: CC(=O)SCC(C)CNC(=O)OCc1ccccc1 ; CC(CNC(=O)OCc1ccccc1)CS(N)(=O)=O]

To a solution of N-chlorosuccinimide (0.93 g, 7.0 mmol, 4.0 eq.) in acetonitrile (29 mL) with hydrochloric acid 2M solution (1.02 mL, 2.04 mmol, 1.2 eq.) at 0° C. was added S-(3-(((benzyloxy)carbonyl)amino)-2-methylpropyl) ethanethioate (0.49 g, 1.7 mmol, I16.3) in acetonitrile (29 mL) dropwise. After stirring at rt for 1 h, ammonia 25% in water (29 mL) was added. After stirring at rt for 2 h, solvents were partially removed under reduced pressure. The residue was diluted with water (20 mL) and extracted with ethyl acetate (50 mL). The organic layers were collected, dried over magnesium sulfate, filtered, and concentrated in vacuo. The residue was purified by flash chromatography eluting with ethyl acetate/heptane mixtures (0/100 to 70/30, v/v). The desired fractions were collected and concentrated in vacuo to give the expected compound as a white solid (0.45 g, 90% yield). LC-MS (ESI+): 286.9 (M+H⁺).

### Synthesis of 3-Amino-2-methylpropane-1-sulfonamide (I16)

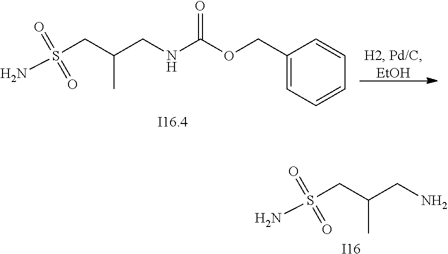

To a solution of benzyl (2-methyl-3-sulfamoylpropyl)carbamate (0.5 g, 1.7 mmol, I16.4) in ethanol (17 mL) was added palladium on carbon 10 wt. % (0.2 g, 0.17 mmol, 0.1 eq.). The reaction mixture was degassed in vacuo and saturated with hydrogen (repeated 3 times). After stirring under hydrogen atmosphere at rt for 4 h, the suspension was filtered through Celite pad and concentrated under reduced pressure to give the expected amine as a brown solid (0.185 g, 70% yield). LC-MS (ESI+): 153.0 (M+H⁺).

### Synthesis of 4-Aminobutane-2-sulfonamide (I17)

### Synthesis of 3-(Benzyloxy)butanenitrile (I17.1)

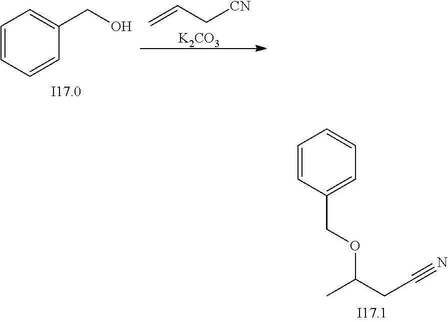

To Potassium tertbutoxide (0.04 g, 0.335 mmol, 0.01 eq.) under nitrogen atmosphere were added benzyl alcohol (3.62 g, 33.45 mmol, 1.0 eq.) and but-3-enenitrile (2.74 mL, 33.45 mmol, 1.0 eq.). After stirring at rt for 16 h, the mixture was neutralized, subjected to aqueous workup dried, filtered, and concentrated. The residue was purified on silica gel using ethyl acetate/heptane mixtures (0/100 to 40/60, v/v) to provide the expected product as a colorless oil (3.56 g, 20.3 mmol, 61% yield). LC-MS (ESI+): 176.2 (M+H⁺).

### Synthesis of Intermediate 3-(Benzyloxy)butan-1-amine (I17.2)

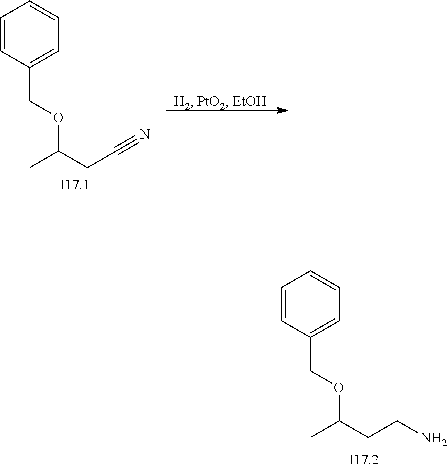

A solution of 3-(Benzyloxy)butanenitrile (3.24 g, 18.5 mmol, 117.1) in ethanol (185 mL) with platinum (IV) oxide (0.21 g, 0.92 mmol, 0.05 eq.) was degassed in vacuo and saturated with hydrogen (repeated 3 times). After stirring under hydrogen atmosphere at rt for 72 h, the suspension was filtered through celite pad and concentrated under reduced pressure to give the expected amine as a colorless oil (3.3 g, 22.1 mmol, quant. yield). LC-MS (ESI+): 180.3 (M+H⁺).

### Synthesis of 2-(3-(benzyloxy)butyl)isoindoline-1,3-dione (I17.3)

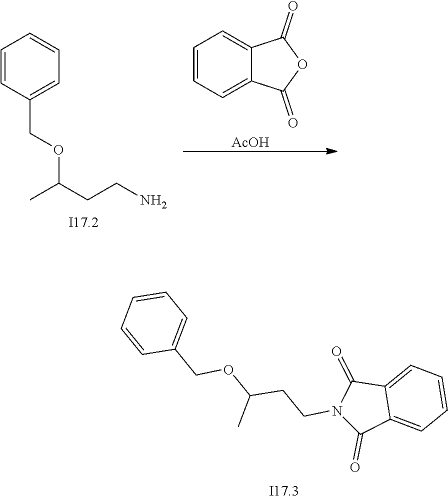 [SMILES: "AcOH" ; CC(CCN)OCc1ccccc1 ; O=C1OC(=O)c2ccccc21 ; CC(CCN1C(=O)c2ccccc2C1=O)OCc1ccccc1]

Phthalic anhydride (3.3 g, 22.1 mmol, 1.1 eq.) and 3-(benzyloxy)butan-1-amine (3.6 g, 20.1 mmol, 117.2) were dissolved in acetic acid. After stirring at refluxed for 16 h, solvent was removed in vacuo. The residue was purified by flash column chromatography on silica eluting with ethyl acetate/heptane mixtures (0/100 to 20/80, v/v). The desired fractions were collected and concentrated under reduced pressure to give the expected product as a colorless oil (3.05 g, 9.8 mmol, 49% yield). LC-MS (ESI+): 309.9 (M+H⁺).

### Synthesis of 2-(3-hydroxybutyl)isoindoline-1,3-dione (I17.4)

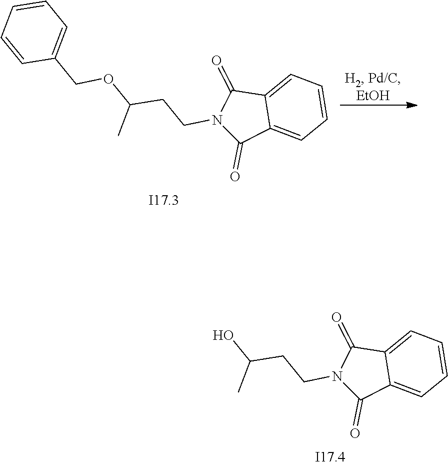 [SMILES: CC(CCN1C(=O)c2ccccc2C1=O)OCc1ccccc1 ; CC(O)CCN1C(=O)c2ccccc2C1=O]

A solution of 2-(3-(benzyloxy)butyl)isoindoline-1,3-dione (3.05 g, 9.8 mmol, 117.3) in ethanol (100 mL) with palladium on carbon (10 wt. %) (1.0 g, 0.98 mmol, 0.1 eq.) was degassed in vacuo and then saturated with hydrogen, repeated 3 times. After stirring at rt for 4 h, the suspension was filtered through Celite pad and concentrated under reduced pressure to give the expected alcohol as a brown solid (2.1 g, 9.7 mmol, quant. yield). LC-MS (ESI+): 219.9 (M+H⁺).

### Synthesis of 4-(1,3-Dioxoisoindolin-2-yl)butan-2-yl methanesulfonate (I17.5)

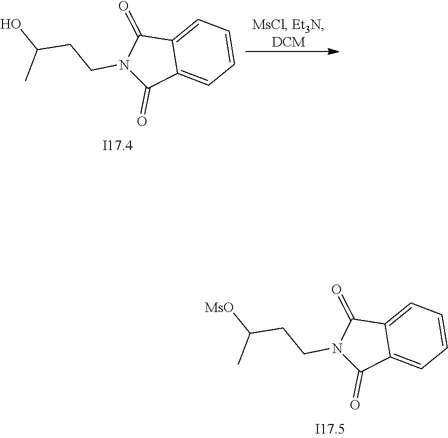 [SMILES: CC(O)CCN1C(=O)c2ccccc2C1=O ; CC(CCN1C(=O)c2ccccc2C1=O)OS(C)(=O)=O]

To a mixture of 2-(3-hydroxybutyl)isoindoline-1,3-dione (2.1 g, 9.7 mmol, 117.4) and triethylamine (1.48 mL, 10.6 mmol, 1.1 eq.) in dichloromethane (32 mL) was added methane sulfonyl chloride (0.79 mL, 10.2 mmol, 1.05 eq.) at 0° C. After stirring at rt for 1 h, the mixture was diluted with dichloromethane (20 mL) and extracted with ammonium chloride (30 mL). The organic layers were separated, dried over magnesium sulfate, filtered, and concentrated in vacuo. The residue was purified by flash column chromatography on silica eluting with ethyl acetate/heptane mixtures (0/100 to 50/50, v/v), affording the expected compound as a colorless oil (1.3 g, 4.37 mmol, 45% yield). LC-MS (ESI+): 297.9 (M+H⁺).

### Synthesis of S-(4-(1,3-Dioxoisoindolin-2-yl)butan-2-yl) ethanethioate (I17.6)

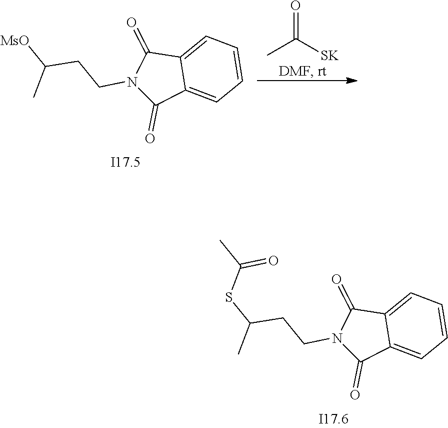

Potassium thioacetate (2.5 g, 21.9 mmol, 5.0 eq.) was added to a solution of 4-(1,3-dioxoisoindolin-2-yl)butan-2-yl methanesulfonate (1.3 g, 4.37 mmol, 117.5) in dimethylformamide (15 mL). After stirring at rt for 18 h, solvent was partially removed under reduced pressure. The residue was diluted with ethyl acetate (40 mL) and washed with water (30 mL) and brine (30 mL). The organic phase was dried over magnesium sulfate, filtered, and concentrated in vacuo. The residue was chromatographed on silica eluting with ethyl acetate/heptane mixtures (0/100 to 70/30, v/v). The desired fractions were collected and concentrated under reduced pressure to afford the expected compound as a yellow oil (0.94 g, 3.4 mmol, 77% yield). LC-MS (ESI+): 277.9 (M+H⁺).

### Synthesis of 4-(1,3-Dioxoisoindolin-2-yl)butane-2-sulfonic acid (I17.7)

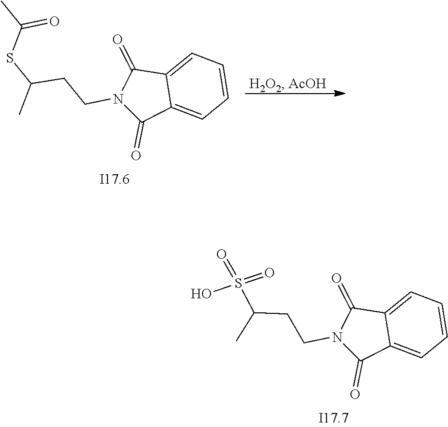 [SMILES: CC(=O)SC(C)CCN1C(=O)c2ccccc2C1=O ; CC(CCN1C(=O)c2ccccc2C1=O)S(=O)(=O)O]

To a solution of S-(4-(1,3-dioxoisoindolin-2-yl)butan-2-yl) ethanethioate (0.94 g, 3.4 mmol, 117.6) in acetic acid (4 mL) was added a mixture of hydrogen peroxide (30% w/w in water) (4 mL) in acetic acid (8 mL). After stirring at rt for 16 h, Palladium on carbon (10% wt, 50% wet) was added to destroy the excess of peroxide. The resulting suspension was filtered through a pad of celite and concentrated in vacuo. The residue was co-evaporated with toluene (×3) and concentrated to dryness under reduced pressure to afford a brown solid which was used in the next step without further purification (0.82 g, 2.9 mmol, 85% yield). LC-MS (ESI+): 283.8 (M+H⁺).

### Synthesis of (1,3-Dioxoisoindolin-2-yl)butane-2-sulfonyl chloride (I17.8)

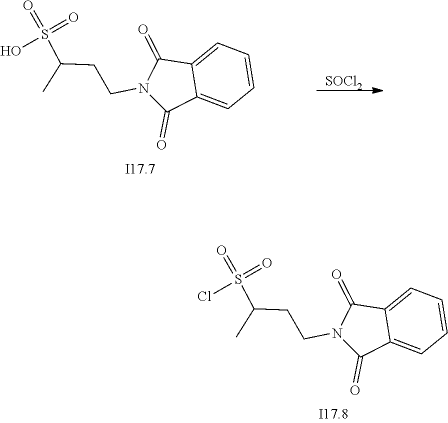 [SMILES: CC(CCN1C(=O)c2ccccc2C1=O)S(=O)(=O)O ; "SOCl2" ; CC(CCN1C(=O)c2ccccc2C1=O)S(=O)(=O)Cl]

A solution of 4-(1,3-dioxoisoindolin-2-yl)butane-2-sulfonic acid (0.82 g, 2.9 mmol, 117.7) in thionyl chloride (10 mL) was refluxed at 80° C. for 16 h. Upon completion, solvent was removed under reduced pressure. The residue was purified by flash chromatography on silica eluting in ethyl acetate/heptane mixtures (0/100 to 50/50, v/v). The desired fractions were collected and concentrated in vacuo to give the expected compound as a colorless oil (0.38 g, 1.25 mmol, 43% yield). LC-MS (ESI+): 301.8 (M+H⁺).

### Synthesis of 4-(1,3-Dioxoisoindolin-2-yl)butane-2-sulfonamide (I17.9)

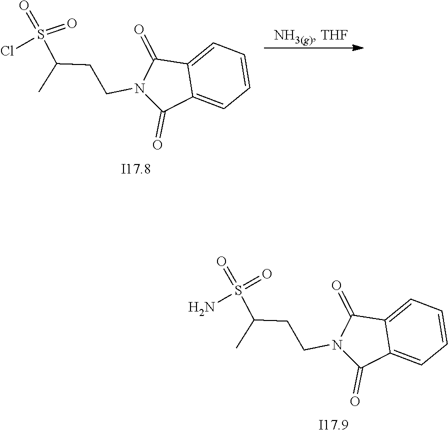

A solution of 4-(1,3-dioxoisoindolin-2-yl)butane-2-sulfonyl chloride (0.38 g, 1.25 mmol, 117.8) in tetrahydrofuran (5 mL) was bubbled with ammonia for 10 min. Upon completion, the reaction mixture was filtered and concentrated in vacuo to afford the expected sulfonamide as a white solid (0.35 g, 1.24 mmol, quant. yield). LC-MS (ESI+): 282.9 (M+H⁺).

### Synthesis of 4-Aminobutane-2-sulfonamide (I17)

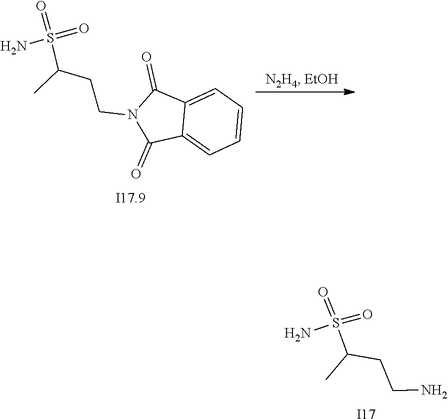

To a solution of 4-(1,3-dioxoisoindolin-2-yl)butane-2-sulfonamide (0.35 g, 1.24 mmol, I17.9) in ethanol (5 mL) was added hydrazine hydrate 50-60% (0.15 mL, 2.48 mmol, 2.0 eq.). After stirring at 80° C. for 1.5 h, the reaction was allowed to cool down and diluted with cold ethanol (10 mL). The precipitate was removed by filtration and the filtrate was concentrated in vacuo affording a white solid which was used in the next step without further purification (0.19 g, 1.25 mmol, quant. yield). LC-MS (ESI+): 153.0 (M+H⁺).

### Synthesis of 4-Aminobutane-1-sulfonamide (I18)

### Synthesis of S-(4-(1,3-Dioxoisoindolin-2-yl)butyl) ethanethioate (I18.1)

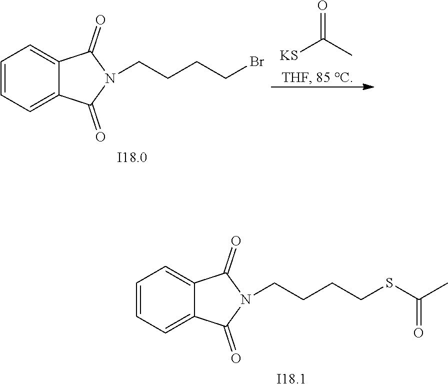

Potassium thioacetate (12.1 g, 106.3 mmol, 3.0 eq.) was added to a stirring solution of N-(4-bromobutyl)phthalimide (10 g, 35.4 mmol, 1.0 eq) in tetrahydrofuran (350 mL). After stirring at 85° C. for 5 h, solvent was removed under reduced pressure. The resulting slurry was diluted with ethyl acetate (75 mL) and washed with water (50 mL). The organic phase was dried over magnesium sulfate, filtered, and concentrated in vacuo. The residue was chromatographed on silica eluting with ethyl acetate/heptane mixtures (0/100 to 30/70, v/v) to afford the expected product as a pink solid (7.3 g, 26.3 mmol, 74% yield). LC-MS (ESI+): 278.0 (M+H⁺).

### Synthesis of 4-(1,3-Dioxoisoindolin-2-yl)butane-1-sulfonic acid (I18.2)

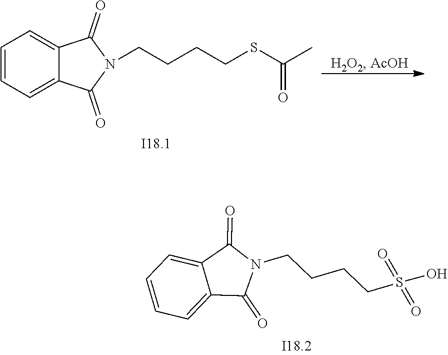 [SMILES: CC(=O)SCCCCN1C(=O)c2ccccc2C1=O ; O=C1c2ccccc2C(=O)N1CCCCS(=O)(=O)O]

A mixture of hydrogen peroxide (30% w/w in water) (23 mL) and acetic acid (46 mL) was added to a solution of S-(4-(1,3-dioxoisoindolin-2-yl)butyl) ethanethioate (7.3 g, 26.3 mmol, 1.0 eq.) in acetic acid (23 mL). After stirring at rt for 16 h, palladium on carbon (10 wt. %) was added to destroy the excess of peroxide. The resulting mixture was filtered through a celite pad and concentrated under reduced pressure. The oily residue was evaporated (×2) with toluene and then concentrated in vacuo to dryness affording a beige solid which was used in the next step without further purification (7.1 g, 25.1 mmol, 96% yield). LC-MS (ESI+): 284.0 (M+H⁺).

### Synthesis of 4-(1,3-dioxoisoindolin-2-yl)butane-1-sulfonyl chloride (I18.3)

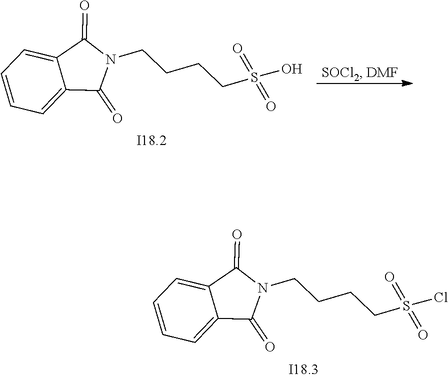 [SMILES: O=C1c2ccccc2C(=O)N1CCCCS(=O)(=O)O ; O=C1c2ccccc2C(=O)N1CCCCS(=O)(=O)Cl]

To a solution of 4-(1,3-dioxoisoindolin-2-yl)butane-1-sulfonic acid (7.18 g, 25.3 mmol, I18.2) in thionyl chloride (25 mL) was added dimethylformamide (1 mL). After stirring at 80° C. for 16 h, solvents were removed under reduced pressure. The residue was diluted with ethyl acetate (60 mL) and washed with sat. sodium bicarbonate solution (45 mL). The organic phase was dried over magnesium sulfate, filtered, and concentrated in vacuo. The residue was purified by flash chromatography on silica eluting with ethyl acetate/heptane mixtures (0/100 to 40/60, v/v) to afford the expected sulfonyl chloride as a beige solid (5.22 g, 17.3 mmol, 68% yield). LC-MS (ESI+): 302.1 (M+H⁺).

### Synthesis of 4-(1,3-Dioxoisoindolin-2-yl)butane-1-sulfonamide (I18.4)

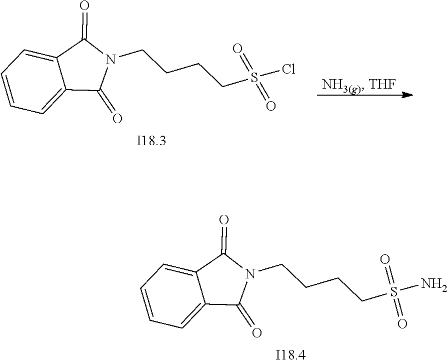 [SMILES: O=C1c2ccccc2C(=O)N1CCCCS(=O)(=O)Cl ; NS(=O)(=O)CCCCN1C(=O)c2ccccc2C1=O]

Ammonia gas was bubbled through a solution of 4-(1,3-dioxoisoindolin-2-yl)butane-1-sulfonyl chloride (5.22 g, 17.3 mmol, 118.3) in tetrahydrofuran (100 mL) for 10-15 min. Upon completion, the reaction mixture was filtered through a pad of celite, washed with more tetrahydrofuran (35 mL) and concentrated in vacuo affording a beige solid which was used in the next step without further purification (4.80 g, 17.0 mmol, 98% yield). LC-MS (ESI+): 283.0 (M+H⁺).

### Synthesis of 4-Aminobutane-1-sulfonamide (I18)

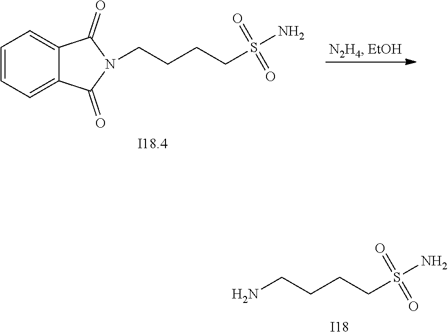 [SMILES: NS(=O)(=O)CCCCN1C(=O)c2ccccc2C1=O ; "I18" ; NCCCCS(N)(=O)=O]

4-(1,3-dioxoisoindolin-2-yl)butane-1-sulfonamide (4.80 g, 17.0 mmol, I18.4) and hydrazine hydrate 50-60% (6.3 mL, 102 mmol, 6.0 eq.) were refluxed in ethanol (170 mL). After stirring at 80° C. for 18 h, the reaction mixture was allowed to cool down and diluted with cold ethanol (50 mL). The filtrate was concentrated under reduced pressure to afford the expected compound as a white solid which was used in the next step without further purification (2.2 g, 14.5 mmol, 85% yield). LC-MS (ESI+): 153.1 (M+H⁺).

### Synthesis of Intermediate 3-(4-chlorophenyl)-4-isopropyl-4,5-dihydro-1H-pyrazole (I19)

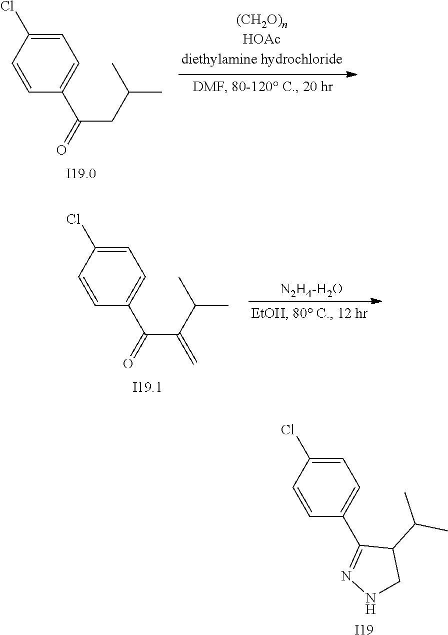

### Synthesis of Intermediate (I19.1)

A mixture of 119.0 (15 g, 76.27 mmol, 1 eq), dimethylamine hydrochloride (24.88 g, 305.08 mmol, 4 eq), acetic acid (1.96 mL, 34.32 mmol, 0.45 eq) and formaldehyde (5.25 mL, 190.67 mmol, 2.5 eq). After stirring at 80° C. for 18 h, the mixture was treated with dimethyl formamide (20 mL) and heated to 120° C. After stirring at 120° C. for 2 hr with microwave reactor, the mixture was cooled to rt, quenched by saturated NH₄Cl (aq, 100 mL), and extracted with dichloromethane (50 mL×3). The combined organic phase was washed with brine (50 mL×2), dried over Na₂SO₄, filtered, and concentrated under reduced pressure to give I19.1 (10 g, 43.13 mmol, 56%) as a yellow oil, which was used directly for the next step, without further purification. LCMS (ESI+): m/z 209.1 (M+H)^{+ 1}H NMR: 400 MHz, CDCl₃ δ ppm 1.05 (d, J=6.79 Hz, 6H), 2.95 (quin, J=6.91 Hz, 1H), 5.38 (s, 11H), 5.66 (d, J=1.31 Hz, 1H), 7.34 (d, J=8.58 Hz, 2H), 7.59-7.70 (m, 2H).

### Synthesis of Intermediate 119

To a solution of 119.1 (3 g, 14.38 mmol, 1 eq) in ethanol (30 mL) at rt was added hydrazine monohydrate (3.49 mL, 71.88 mmol, 5 eq). After stirring at 80° C. for 12 h, the reaction mixture was concentrated under reduced pressure. The residue was triturated with ethanol (20 mL) and filtered to give 119 (1 g, 4.04 mmol, 28%) as a white solid. LCMS (ESI+): m/z 223.1 (M+H)^{+ 1}H NMR: 400 MHz, DMSO-d₆ δ ppm 0.66 (d, J=6.79 Hz, 3H), 0.92 (d, J=6.91 Hz, 3H), 1.96 (td, J=6.85, 3.58 Hz, 1H), 3.35-3.41 (m, 2H), 3.42-3.49 (m, 1H), 7.37-7.45 (m, 2H), 7.60-7.66 (m, 2H).

### Example 2. Synthesis of Compounds

### Synthesis of (R,E)-3-(4-chlorophenyl)-N′-((4-chlorophenyl)sulfonyl)-4-phenyl-N-((S)-1-sulfamoylpropan-2-yl)-4,5-dihydro-1H-pyrazole-1-carboximidamide (Compound 1) & (S,E)-3-(4-chlorophenyl)-N′-((4-chlorophenyl)sulfonyl)-4-phenyl-N-(1-sulfamoylpropan-2-yl)-4,5-dihydro-1H-pyrazole-1-carboximidamide (Compound 2)

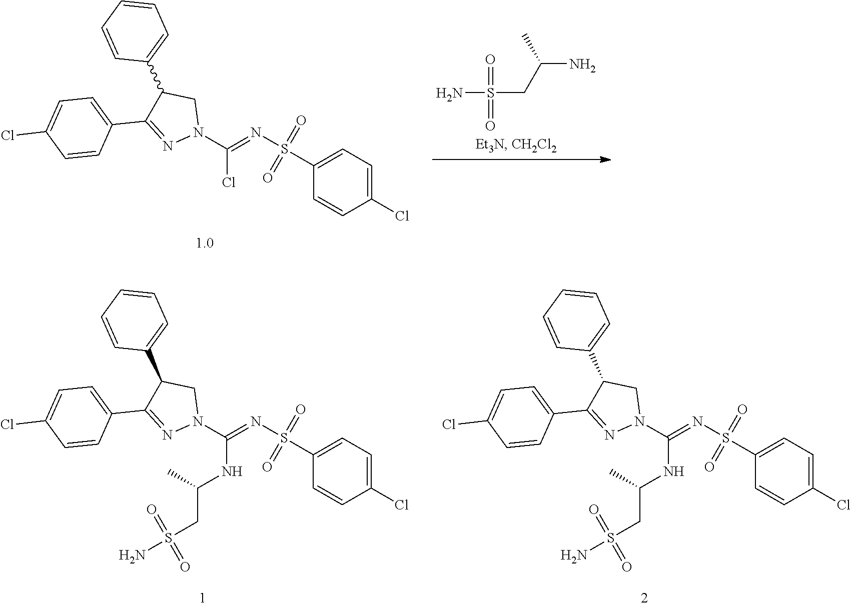

To a solution of (S)-2-aminopropane-1-sulfonamide (105 mg, 0.76 mmol, 1.5 eq., I1) and triethylamine (0.22 mL, 1.52 mmol, 3.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0, CAS #1148142-85-1). After stirring at room temperature for 18 hours, the reaction was diluted with dichloromethane (25 mL) and washed with aqueous HCl (2N, 20 mL). The organic layer was dried over magnesium sulfate, filtered, concentrated in vacuo, and purified by flash chromatography on silica eluting with ethyl acetate/heptane mixtures (0/100 to 80/20, v/v) to give a colorless oil (mixture of both diastereoisomers). A second chromatography was carried out to separate the diastereomers, using a slow gradient of acetate/heptane mixtures (0/100 to 70/30, v/v), obtaining separately both fractions.

Compound 1: (26.1 mg, 0.044 mmol, 9% yield, 97% purity, 94% de, OR: −35.4). ¹H NMR (DMSO-d₆, 400 MHz) δ 7.98 (br s, 1H), 7.8-7.9 (m, 2H), 7.72 (d, J=8.6 Hz, 2H), 7.5-7.6 (m, 2H), 7.45 (d, J=8.6 Hz, 2H), 7.3-7.4 (m, 2H), 7.2-7.3 (m, 3H), 6.93 (br s, 2H), 5.10 (dd, J=4.4, 11.2 Hz, 1H), 4.53 (t, J=11.2 Hz, 1H), 4.40 (br s, 1H), 4.13 (br dd, J=3.9, 11.0 Hz, 1H), 3.4 (m, 1H), 3.22 (dd, J=6.3, 14.1 Hz, 1H), 1.28 (d, J=6.7 Hz, 3H). LC-MS (ESI+): 594.0 (M+H⁺).

Compound 2: (46.7 mg, 0.08 mol, 16% yield, 99% purity, 99% ee, OR: +116.8). ¹H NMR (DMSO-d₆, 400 MHz) δ 7.96 (br d, J=8.3 Hz, 1H), 7.8-7.9 (m, 2H), 7.74 (d, J=8.7 Hz, 2H), 7.5-7.6 (m, 2H), 7.45 (d, J=8.6 Hz, 2H), 7.3-7.4 (m, 2H), 7.2-7.3 (m, 3H), 6.95 (s, 2H), 5.09 (dd, J=4.3, 11.2 Hz, 1H), 4.58 (t, J=11.3 Hz, 1H), 4.3-4.5 (m, 1H), 4.08 (br dd, J=4.2, 11.2 Hz, 1H), 3.44 (dd, J=6.3, 14.1 Hz, 1H), 3.24 (dd, J=6.1, 14.1 Hz, 1H), 1.26 (d, J=6.7 Hz, 3H). LC-MS (ESI+): 594.0 (M+H⁺).

### (R,E)-3-(4-chlorophenyl)-N′-((4-chlorophenyl)sulfonyl)-4-phenyl-N-((R)-1-sulfamoylpropan-2-yl)-4,5-dihydro-1H-pyrazole-1-carboximidamide (Compound 3) & (S,E)-3-(4-chlorophenyl)-N′-((4-chlorophenyl)sulfonyl)-4-phenyl-N-((R)-1-sulfamoylpropan-2-yl)-4,5-dihydro-1H-pyrazole-1-carboximidamide (Compound 4)

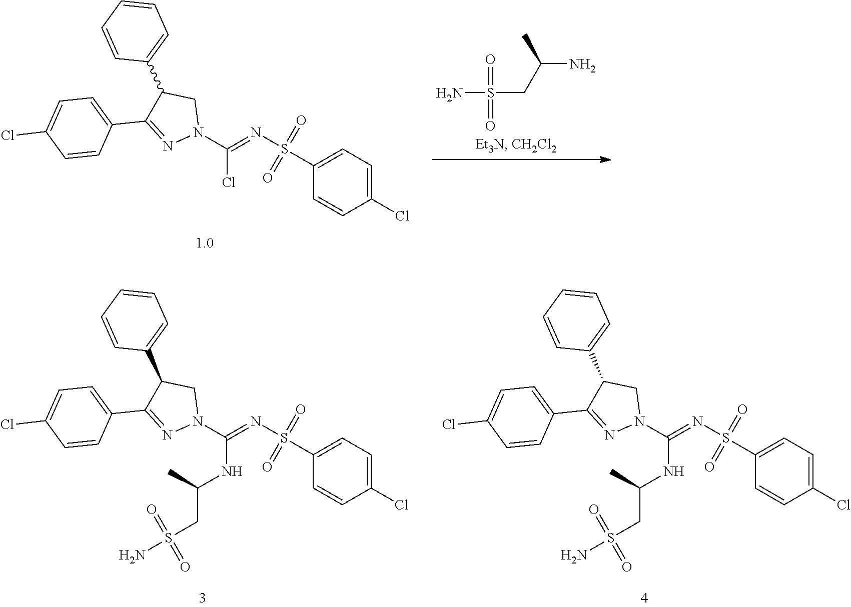

To a solution of (R)-2-aminopropane-1-sulfonamide (126 mg, 0.91 mmol, 1.5 eq.) and triethylamine (0.26 mL, 1.83 mmol, 3.0 eq.) in dichloromethane (3 mL) was added (E)-3-(4-chlorophenyl)-N-((4-chlorophenyl)sulfonyl)-4-phenyl-4,5-dihydro-1H-pyrazole-1-carbimidoyl chloride (300 mg, 0.61 mmol, 1.0). After stirring at room temperature for 18 hours, solvents were removed under reduced pressure and the crude was purified by flash chromatography on silica eluting with ethyl acetate/heptane mixtures (0/100 to 80/20, v/v) to give a colorless oil, which was further subjected to SFC separation, giving the following 2 different diastereomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%)—iPrOH+0.1% diethylamine, at 35° C., BPR 100 bar. Acquisition frequency was set to 220 nm for the DAD detector.

Compound 3: (32.0 mg, 0.05 mmol, 9% yield, 99% purity, 99% ee, OR: −79.1). ¹H NMR (DMSO-d₆, 400 MHz) δ 7.96 (br d, J=1.1 Hz, 1H), 7.8-7.9 (m, 2H), 7.74 (d, J=8.6 Hz, 2H), 7.5-7.6 (m, 2H), 7.45 (d, J=8.6 Hz, 2H), 7.3-7.4 (m, 2H), 7.2-7.3 (m, 3H), 6.96 (br s, 2H), 5.09 (dd, J=4.3, 11.2 Hz, 1H), 4.58 (t, J=11.2 Hz, 1H), 4.41 (br s, 1H), 4.09 (br dd, J=4.1, 11.1 Hz, 1H), 3.44 (dd, J=6.3, 14.1 Hz, 1H), 3.25 (dd, J=6.1, 14.1 Hz, 1H), 1.26 (d, J=6.7 Hz, 3H). LC-MS (ESI+): 596.1 (M+H⁺).

Compound 4: (28.1 mg, 0.05 mol, 8% yield, 99% purity, 99% ee, OR: +48.4). ¹H NMR (DMSO-d₆, 400 MHz) δ 7.98 (br d, J=7.8 Hz, 1H), 7.82 (d, J=8.6 Hz, 2H), 7.72 (d, J=8.6 Hz, 2H), 7.53 (d, J=8.6 Hz, 2H), 7.45 (d, J=8.7 Hz, 2H), 7.33 (s, 2H), 7.2-7.3 (m, 3H), 6.93 (s, 2H), 5.10 (dd, J=4.2, 11.1 Hz, 1H), 4.53 (t, J=11.2 Hz, 1H), 4.40 (br d, J=4.0 Hz, 1H), 4.13 (br dd, J=3.5, 11.0 Hz, 1H), 3.37 (br d, J=6.3 Hz, 1H), 3.2 (m, 1H), 1.28 (d, J=6.6 Hz, 3H). LC-MS (ESI+): 596.0 (M+H⁺).

### Synthesis of (S,E)-3-(4-chlorophenyl)-N′-((4-chlorophenyl)sulfonyl)-4-phenyl-N-((R)-2-sulfamoylpropyl)-4,5-dihydro-1H-pyrazole-1-carboximidamide (Compound 5), (S,E)-3-(4-chlorophenyl)-N′-((4-chlorophenyl)sulfonyl)-4-phenyl-N-((S)-2-sulfamoylpropyl)-4,5-dihydro-1H-pyrazole-1-carboximidamide (Compound 6), (R,E)-3-(4-chlorophenyl)-N′-((4-chlorophenyl)sulfonyl)-4-phenyl-N-((R)-2-sulfamoylpropyl)-4,5-dihydro-1H-pyrazole-1-carboximidamide (Compound 7) & (R,E)-3-(4-chlorophenyl)-N′-((4-chlorophenyl)sulfonyl)-4-phenyl-N-((S)-2-sulfamoylpropyl)-4,5-dihydro-1H-pyrazole-1-carboximidamide (Compound 8)

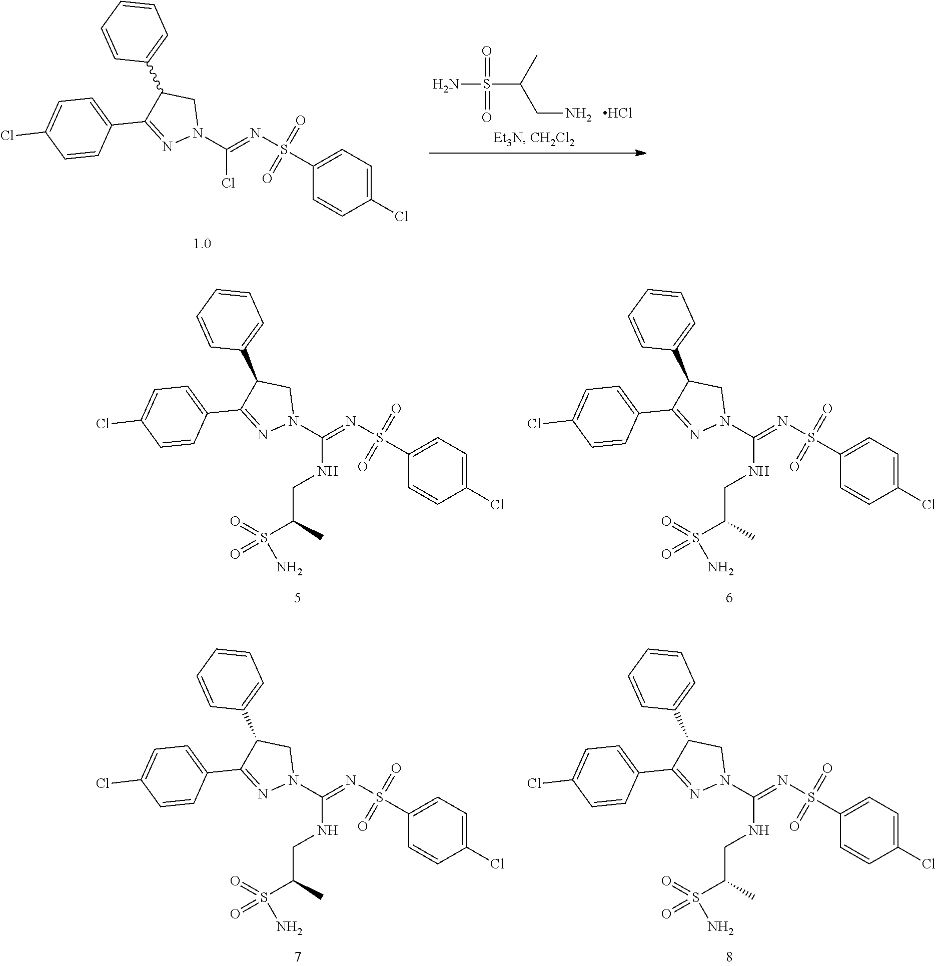

To a stirred solution of 1-aminopropane-2-sulfonamide hydrochloride (0.18 g, 1.02 mmol, 1.5 eq.) and triethylamine (0.28 mL, 2.05 mmol, 3.0 eq.) in dichloromethane (3.5 mL) was added (E)-3-(4-chlorophenyl)-N-((4-chlorophenyl)sulfonyl)-4-phenyl-4,5-dihydro-1H-pyrazole-1-carbimidoyl chloride (0.34 g, 0.68 mmol, 1.0). After stirring at room temperature for 18 hours, the reaction was diluted with dichloromethane (20 mL) and washed with ammonium chloride (15 mL). The organic layer was dried over magnesium sulfate, filtered, and concentrated in vacuo. The residue was purified by flash chromatography on silica eluting with heptane/ethyl acetate mixtures (100/0 to 60/40, v/v) to give a colorless oil as a mixture, which was further subjected to SFC separation to afford the four isomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 2.5 mL/min of CO2 (60%)—MeOH+0.1% diethylamine, at 35° C., BPR 100 bar. Acquisition frequency was set to 220 nm for the DAD detector. Absolute stereochemistry was not determined. Diastereomers with (-) OR were arbitrary assigned as S isomer.

Compound 5: (16.1 mg, 0.03 mmol, 4% yield, 99% purity, 99% ee, OR: −63.0). ¹H NMR (CDCl₃, 400 MHz) δ 7.85 (br d, J=8.3 Hz, 2H), 7.66 (br s, 1H), 7.53 (br d, J=8.3 Hz, 2H), 7.39 (br d, J=8.3 Hz, 2H), 7.3-7.4 (m, 3H), 7.25 (s, 2H), 7.12 (br d, J=7.0 Hz, 2H), 4.95 (br s, 2H), 4.70 (br dd, J=4.4, 10.2 Hz, 1H), 4.53 (br t, J=11.2 Hz, 1H), 3.9-4.1 (m, 3H), 3.51 (br s, 1H), 1.46 (br d, J=6.2 Hz, 3H). LC-MS (ESI+): 594.1 (M+H⁺).

Compound 6: (27.0 mg, 0.045 mmol, 7% yield, 99% purity, 99% ee, OR: −97.8). ¹H NMR (DMSO-d₆, 400 MHz) δ 8.13 (br s, 1H), 7.7-7.9 (m, 4H), 7.4-7.6 (m, 4H), 7.2-7.4 (m, 5H), 6.99 (br s, 2H), 5.11 (br d, J=7.1 Hz, 1H), 4.55 (br t, J=11.0 Hz, 1H), 4.13 (br d, J=7.2 Hz, 1H), 3.70 (br d, J=12.5 Hz, 1H), 3.56 (br d, J=6.4 Hz, 1H), 2.8-3.0 (m, 1H), 1.17 (br d, J=6.5 Hz, 3H). LC-MS (ESI+): 594.0 (M+H⁺).

Compound 7: (52.1 mg, 0.09 mmol, 13% yield, 98% purity, 99% ee, OR: +111.1). ¹H NMR (DMSO-d₆, 400 MHz) δ 8.1-8.2 (m, 1H), 7.83 (d, J=8.6 Hz, 2H), 7.74 (d, J=8.6 Hz, 2H), 7.54 (d, J=8.5 Hz, 2H), 7.47 (d, J=8.6 Hz, 2H), 7.3-7.4 (m, 2H), 7.2-7.3 (m, 3H), 6.98 (s, 2H), 5.11 (dd, J=4.3, 11.2 Hz, 1H), 4.58 (t, J=11.2 Hz, 1H), 4.08 (dd, J=4.2, 11.1 Hz, 1H), 3.7 (m, 1H), 3.57 (td, J=6.8, 13.9 Hz, 1H), 3.2-3.3 (m, 1H), 1.18 (d, J=7.0 Hz, 3H). LC-MS (ESI+): 594.0 (M+H⁺).

Compound 8: (55.2 mg, 0.093 mmol, 14% yield, 99% purity, 99% ee, OR: +102.2). ¹H NMR (DMSO-d₆, 400 MHz) δ 8.07 (br s, 1H), 7.7-7.8 (m, 2H), 7.66 (d, J=8.7 Hz, 2H), 7.5 (m, 2H), 7.39 (d, J=8.7 Hz, 2H), 7.2-7.3 (m, 2H), 7.2 (m, 3H), 6.92 (s, 2H), 5.04 (dd, J=4.4, 11.2 Hz, 1H), 4.48 (t, J=11.1 Hz, 1H), 4.07 (br dd, J=4.2, 11.0 Hz, 1H), 3.63 (br d, J=13.3 Hz, 1H), 3.4-3.5 (m, 1H), 3.2 (m, 1H), 1.10 (d, J=6.9 Hz, 3H). LC-MS (ESI+): 594.0 (M+H⁺).

### Synthesis of (S,E)-3-(4-chlorophenyl)-4-phenyl-N-(2-sulfamoylethyl)-N′-((4-(trifluoromethyl)phenyl)sulfonyl)-4,5-dihydro-1H-pyrazole-1-carboximidamide (Compound 9) & (R,E)-3-(4-chlorophenyl)-4-phenyl-N-(2-sulfamoylethyl)-N′-((4-(trifluoromethyl)phenyl)sulfonyl)-4,5-dihydro-1H-pyrazole-1-carboximidamide (Compound 10)

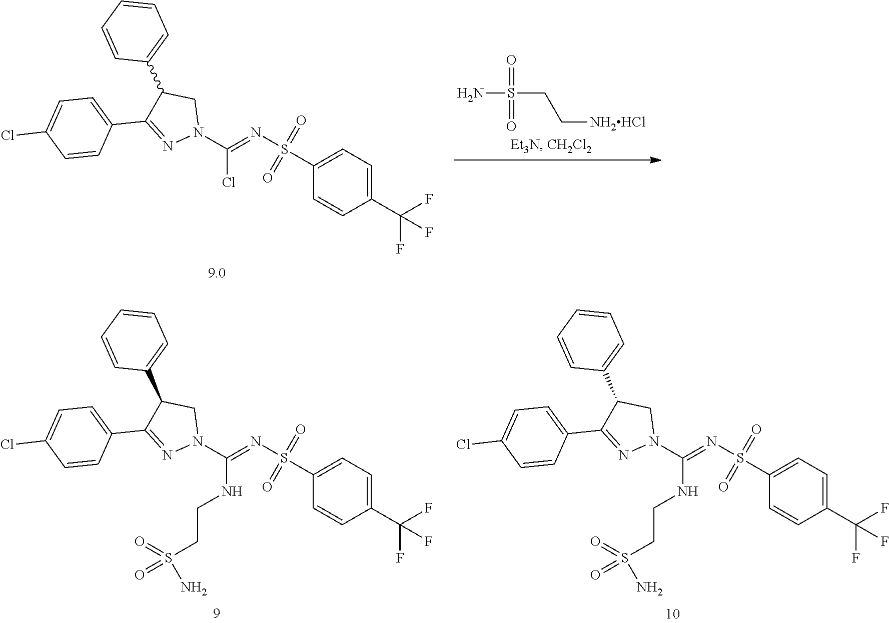

To a stirred solution 2-aminoethane-1-sulfonamide hydrochloride (84 mg, 0.53 mmol, 1.1 eq.) and triethylamine (0.2 mL, 1.42 mmol, 3.0 eq.) in dichloromethane (5 mL) at rt was added (Z)-3-(4-chlorophenyl)-4-phenyl-N-((4-(trifluoromethyl)phenyl)sulfonyl)-4,5-dihydro-1H-pyrazole-1-carbimidoyl chloride (0.25 g, 0.47 mmol, 9.0, CAS #2561196-52-7). After stirring at rt for 18 h, the reaction was diluted with dichloromethane (20 mL) and washed with HCl (2N, 15 mL). The organic layer was dried over magnesium sulfate, filtered, and concentrated in vacuo. The residue was purified by flash chromatography on silica eluting with methanol/dichloromethane mixtures (0/100 to 10/90, v/v) to give a colorless oil. This racemic mixture was further subjected to SFC separation conditions.

SFC purification was performed on a Jasco 4000 Parallel SFC System (4× Channels 3×UV+1×DAD) using an Phenomenex column (Cellulose-4) 100 mm long×4.6 mm I.D. 3 μm particle size, on isocratic mode at 2.5 ml/min of CO₂ (40%)—iPrOH+0.1% diethylamine, at 35° C., BPR 100 bar. Acquisition frequency was set to 220 nm for the DAD detector.

Compound 9: (54.1 mg, 0.09 mmol, 19% yield, 98% purity, 99% ee). ¹H NMR (400 MHz, DMSO) δ 8.23 (s, 1H), 8.03 (d, J=8.2 Hz, 2H), 7.84 (d, J=8.3 Hz, 2H), 7.70 (d, J=8.3 Hz, 2H), 7.44 (d, J=8.6 Hz, 2H), 7.33 (t, J=7.3 Hz, 2H), 7.25 (ddd, J=11.6, 6.7, 4.1 Hz, 3H), 7.02 (s, 2H), 5.09 (dd, J=11.0, 4.1 Hz, 1H), 4.54 (t, J=11.1 Hz, 1H), 4.05 (dd, J=11.0, 4.2 Hz, 1H), 3.73 (t, J=6.6 Hz, 2H), 3.35-3.20 (m, 2H). LC-MS (ESI+): 614.0 (M+H⁺).

Compound 10: (57.8 mg, 0.09 mmol, 20% yield, 99% purity, 99% ee). ¹H NMR (400 MHz, DMSO) δ 8.23 (s, 1H), 8.03 (d, J=8.2 Hz, 2H), 7.84 (d, J=8.3 Hz, 2H), 7.70 (d, J=8.6 Hz, 2H), 7.45 (d, J=8.7 Hz, 2H), 7.33 (t, J=7.3 Hz, 2H), 7.25 (ddd, J=11.4, 6.7, 4.1 Hz, 3H), 7.02 (s, 2H), 5.10 (dd, J=11.1, 4.3 Hz, 1H), 4.54 (t, J=11.1 Hz, 1H), 4.05 (dd, J=10.9, 4.1 Hz, 1H), 3.73 (t, J=6.7 Hz, 2H), 3.30-3.23 (m, 2H). LC-MS (ESI+): 614.0 (M+H⁺).

### Synthesis of (S,E)-3-(4-chlorophenyl)-N′-((4-fluorophenyl)sulfonyl)-4-phenyl-N-(2-sulfamoylethyl)-4,5-dihydro-1H-pyrazole-1-carboximidamide (Compound 11) & (R,E)-3-(4-chlorophenyl)-N′-((4-fluorophenyl)sulfonyl)-4-phenyl-N-(2-sulfamoylethyl)-4,5-dihydro-1H-pyrazole-1-carboximidamide (Compound 12)

### Synthesis of Compound 11.1

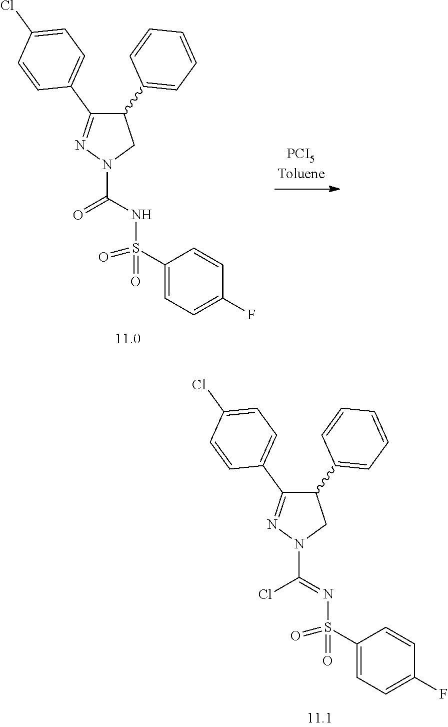 [SMILES: O=C(NS(=O)(=O)c1ccc(F)cc1)N1CC(c2ccccc2)C(c2ccc(Cl)cc2)=N1 ; O=S(=O)(/N=C(\Cl)N1CC(c2ccccc2)C(c2ccc(Cl)cc2)=N1)c1ccc(F)cc1]

To a solution of 3-(4-chlorophenyl)-N-((4-fluorophenyl)sulfonyl)-4-phenyl-4,5-dihydro-1H-pyrazole-1-carboxamide (0.9 g, 1.96 mmol, 11.0, CAS #656827-60-0) in toluene (6 mL) at rt was added phosphorus pentachloride (0.45 g, 2.16 mmol, 1.1 eq). After stirring at 120° C. for 4 hr, solvent was partially removed under reduced pressure, quenched with water (10 mL) and extracted with ethyl acetate (20 mL). The organic layer was separated, dried over magnesium sulfate, filtered and concentrated in vacuo to give a brown foam (0.73 g, 1.54 mmol, 78% yield). LC-MS (ESI+): 476.040 (M+H⁺).

### Synthesis of Compounds 11 and 12

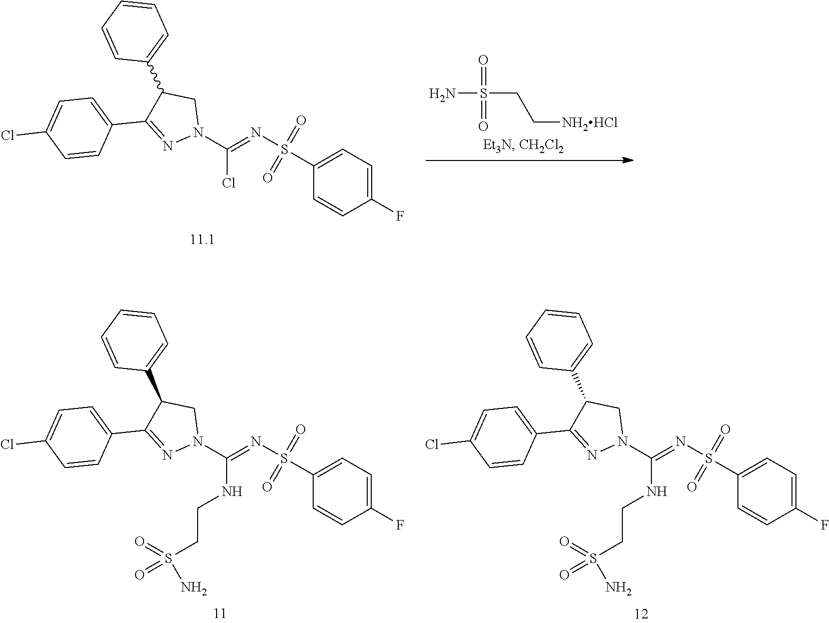

To a stirred solution 2-aminoethane-1-sulfonamide hydrochloride (0.1 g, 0.63 mmol, 1.5 eq.) and triethylamine (0.2 mL, 1.4 mmol, 3.0 eq.) in dichloromethane (4 mL) was added (E)-3-(4-chlorophenyl)-N-((4-fluorophenyl)sulfonyl)-4-phenyl-4,5-dihydro-1H-pyrazole-1-carbimidoyl chloride (0.2 g, 0.42 mmol, 11.1). After stirring at room temperature for 18 hours, the reaction was diluted with dichloromethane (20 mL) and washed with aqueous ammonium chloride solution (15 mL). The organic layer was dried over magnesium sulfate, filtered, and concentrated in vacuo. The residue was purified by flash chromatography on silica gel eluting with heptane/ethyl acetate mixtures (100/0 to 50/50, v/v) to give a colorless oil as a racemic mixture which were further subjected to SFC separation conditions:

SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 2.5 ml/min of CO₂ (60%)—MeOH+0.1% diethylamine, at 35° C., BPR 100 bar. Acquisition frequency was set to 220 nm for the DAD detector.

Compound 11: (21.4 mg, 0.04 mmol, 9% yield, 99% purity, 99% ee, OR: −114.8). ¹H NMR (400 MHz, CDCl₃) δ 7.92 (dd, J=5.1, 8.8 Hz, 2H), 7.65 (br s, 1H), 7.52 (d, J=8.6 Hz, 2H), 7.3 (m, 3H), 7.2 (m, 2H), 7.1 (m, 4H), 5.37 (br s, 2H), 4.67 (dd, J=5.3, 11.3 Hz, 1H), 4.53 (t, J=11.5 Hz, 1H), 4.18 (q, J=5.9 Hz, 2H), 4.05 (dd, J=5.4, 11.5 Hz, 1H), 3.54 (br t, J=5.8 Hz, 2H). LC-MS (ESI+): 564.0 (M+H⁺).

Compound 12: (19.8 mg, 0.035 mmol, 8% yield, 97% purity, 99% ee, OR: +85.1). ¹H NMR (400 MHz, CDCl₃) δ 7.93 (dd, J=5.1, 8.8 Hz, 2H), 7.62 (br s, 1H), 7.53 (d, J=8.5 Hz, 2H), 7.4 (m, 3H), 7.3 (m, 2H), 7.1 (m, 4H), 5.14 (br s, 2H), 4.70 (dd, J=5.3, 11.3 Hz, 1H), 4.55 (t, J=11.4 Hz, 1H), 4.20 (q, J=5.8 Hz, 2H), 4.07 (dd, J=5.3, 11.5 Hz, 1H), 3.54 (br t, J=5.8 Hz, 2H). LC-MS (ESI+): 564.1 (M+H⁺).

### Synthesis of (S,E)-3-(4-chlorophenyl)-N′-((4-fluorophenyl)sulfonyl)-4-phenyl-N-((S)-2-sulfamoylpropyl)-4,5-dihydro-1H-pyrazole-1-carboximidamide (Compound 13), (S,E)-3-(4-chlorophenyl)-N′-((4-fluorophenyl)sulfonyl)-4-phenyl-N-((R)-2-sulfamoylpropyl)-4,5-dihydro-1H-pyrazole-1-carboximidamide (Compound 14), (R,E)-3-(4-chlorophenyl)-N′-((4-fluorophenyl)sulfonyl)-4-phenyl-N-((S)-2-sulfamoylpropyl)-4,5-dihydro-1H-pyrazole-1-carboximidamide (Compound 15) & (R,E)-3-(4-chlorophenyl)-N′-((4-fluorophenyl)sulfonyl)-4-phenyl-N-((R)-2-sulfamoylpropyl)-4,5-dihydro-1H-pyrazole-1-carboximidamide (Compound 16)

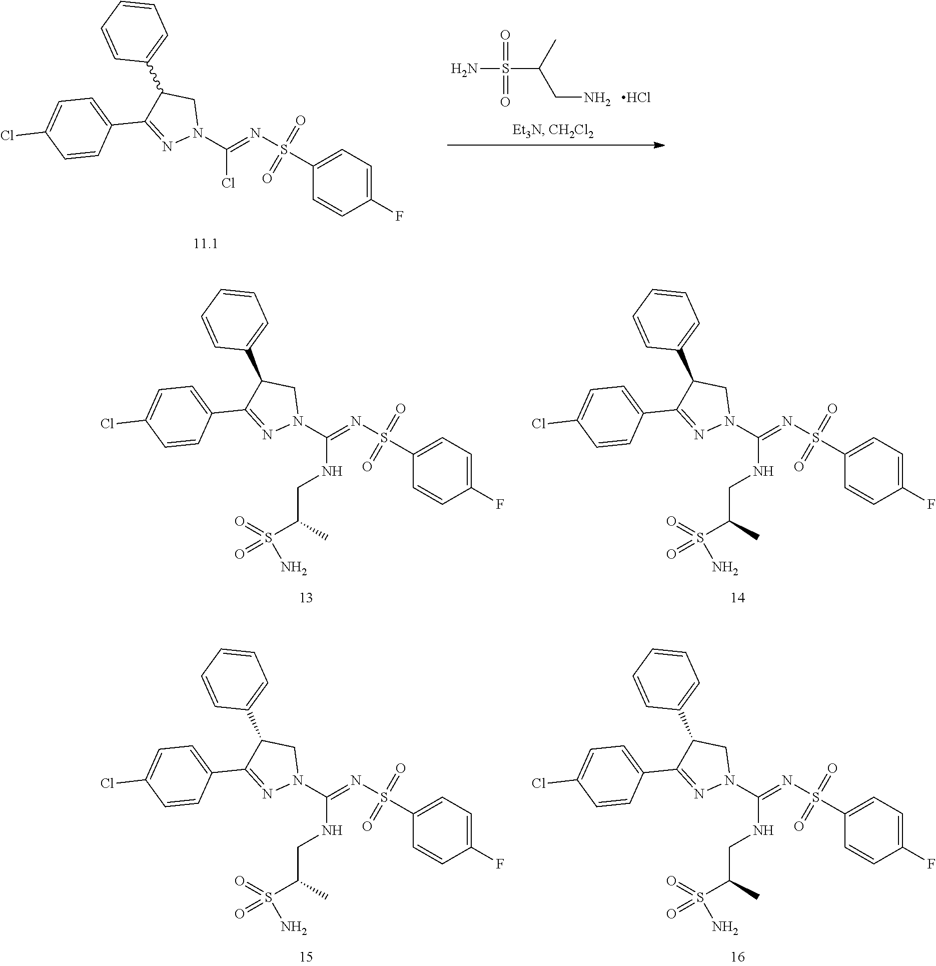

To a stirred solution of 1-aminopropane-2-sulfonamide hydrochloride (0.21 g, 1.3 mmol, 1.5 eq.) and triethylamine (0.33 mL, 2.4 mmol, 3.0 eq.) in dichloromethane (4 mL) was added (E)-3-(4-chlorophenyl)-N-((4-fluorophenyl)sulfonyl)-4-phenyl-4,5-dihydro-1H-pyrazole-1-carbimidoyl chloride (0.38 g, 0.8 mmol, 11.1). After stirring at room temperature for 18 hours, the reaction was diluted with dichloromethane (20 mL) and washed with aqueous ammonium chloride solution (15 mL). The organic layer was dried over magnesium sulfate, filtered, and concentrated in vacuo. The residue was purified by flash chromatography on silica gel eluting with heptane/ethyl acetate mixtures (100/0 to 60/40, v/v) to give a colorless oil as a racemic mixture which were further subjected to SFC separation conditions.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 2.5 ml/min of CO2 (60%)—MeOH+0.1% diethylamine, at 35° C., BPR 100 bar. Acquisition frequency was set to 220 nm for the DAD detector. Absolute stereochemistry was not determined. Diastereomers with (-) OR were arbitrary assigned as S isomer.

Compound 13: (14.3 mg, 0.025 mmol, 3% yield, 97% purity, 99% ee, OR: −99.2). ¹H NMR (400 MHz, CDCl₃) δ 7.9 (m, 2H), 7.62 (br t, J=6.0 Hz, 1H), 7.55 (m, 2H), 7.3 (m, 3H), 7.2 (m, 2H), 7.1 (m, 4H), 4.95 (br s, 2H), 4.68 (dd, J=5.4, 11.4 Hz, 1H), 4.5 (m, 1H), 4.1 (m, 3H), 3.5 (m, 1H), 1.47 (d, J=7.1 Hz, 3H). LC-MS (ESI+): 578.1 (M+H⁺).

Compound 14: (12.5 mg, 0.022 mmol, 3% yield, 96% purity, 99% ee, OR: −120.8). ¹H NMR (400 MHz, CDCl₃) δ 7.9 (m, 2H), 7.62 (br t, J=6.0 Hz, 1H), 7.55 (m, 2H), 7.3 (m, 3H), 7.2 (m, 2H), 7.1 (m, 4H), 4.94 (br s, 2H), 4.69 (dd, J=5.4, 11.4 Hz, 1H), 4.54 (t, J=11.5 Hz, 1H), 3.9-4.1 (m, 3H), 3.5 (m, 1H), 1.46 (d, J=7.0 Hz, 3H). LC-MS (ESI+): 578.1 (M+H⁺).

Compound 15: (7.7 mg, 0.013 mmol, 2% yield, 99% purity, 99% ee, OR: +87.05). ¹H NMR (400 MHz, CDCl₃) δ 7.95 (m, 2H), 7.62 (br t, J=6.0 Hz, 1H), 7.55 (m, 2H), 7.35 (m, 3H), 7.25 (m, 2H), 7.1 (m, 4H), 4.92 (br s, 2H), 4.69 (dd, J=5.3, 11.4 Hz, 1H), 4.55 (m, 1H), 3.9-4.1 (m, 3H), 3.51 (dt, J=4.0, 7.2 Hz, 1H), 1.47 (d, J=7.0 Hz, 3H). LC-MS (ESI+): 578.1 (M+H⁺).

Compound 16: (9.7 mg, 0.017 mmol, 2% yield, 99% purity, 99% ee, OR: +77.4). ¹H NMR (400 MHz, CDCl₃) δ 7.95 (m, 2H), 7.61 (br t, J=6.0 Hz, 1H), 7.55 (m, 2H), 7.35 (m, 3H), 7.25 (m, 2H), 7.15 (m, 4H), 4.88 (br s, 2H), 4.69 (dd, J=5.4, 11.4 Hz, 1H), 4.54 (t, J=11.5 Hz, 1H), 3.9-4.2 (m, 3H), 3.51 (dt, J=4.1, 7.2 Hz, 1H), 1.47 (d, J=7.1 Hz, 3H,). LC-MS (ESI+): 578.1 (M+H⁺).

### Synthesis of (S,E)-3-(4-chlorophenyl)-4-phenyl-N-((R)-2-sulfamoylpropyl)-N′-((4-(trifluoromethyl)phenyl)sulfonyl)-4,5-dihydro-1H-pyrazole-1-carboximidamide (Compound 17), (S,E)-3-(4-chlorophenyl)-4-phenyl-N-((S)-2-sulfamoylpropyl)-N′-((4-(trifluoromethyl)phenyl)sulfonyl)-4,5-dihydro-1H-pyrazole-1-carboximidamide (Compound 18), (R,E)-3-(4-chlorophenyl)-4-phenyl-N-((R)-2-sulfamoylpropyl)-N′-((4-(trifluoromethyl)phenyl)sulfonyl)-4,5-dihydro-1H-pyrazole-1-carboximidamide (Compound 19), & (R,E)-3-(4-chlorophenyl)-4-phenyl-N-((S)-2-sulfamoylpropyl)-N′-((4-(trifluoromethyl)phenyl)sulfonyl)-4,5-dihydro-1H-pyrazole-1-carboximidamide (Compound 20)

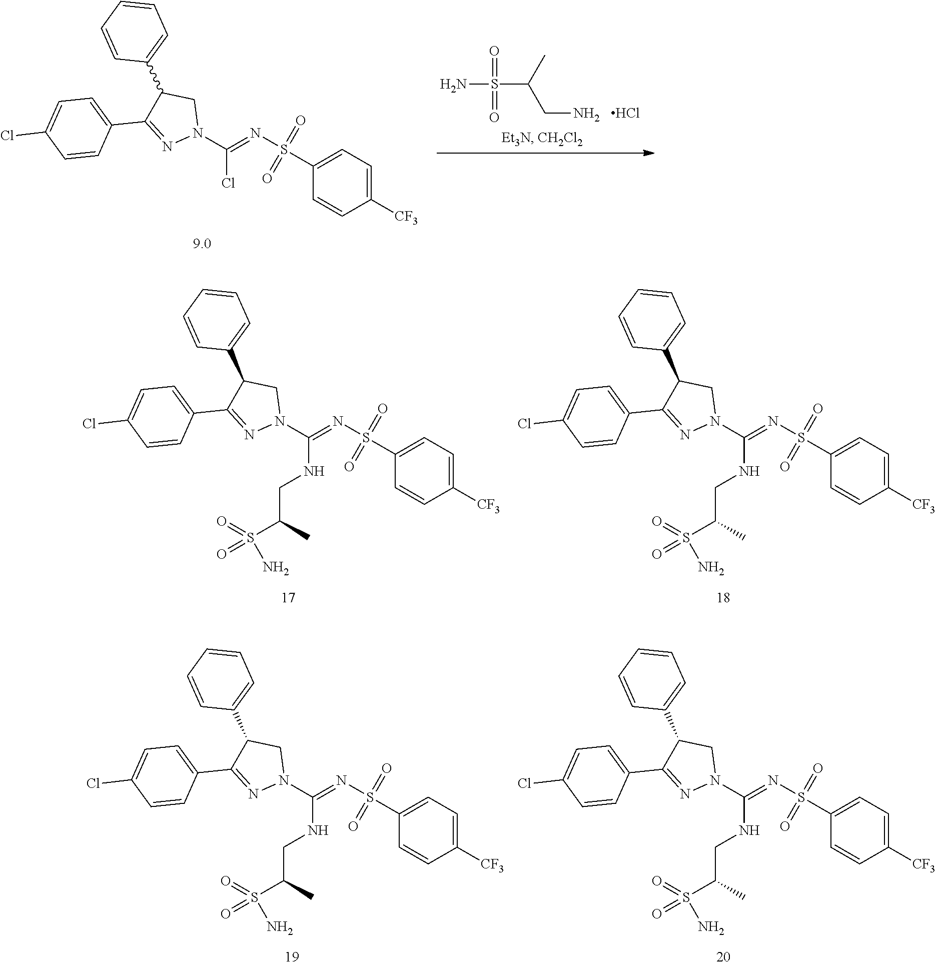

To a stirred solution of 1-aminopropane-2-sulfonamide hydrochloride (0.114 g, 0.65 mmol, 1.5 eq.) and triethylamine (0.18 mL, 1.3 mmol, 3.0 eq.) in dichloromethane (3 mL) at rt was added (E)-3-(4-chlorophenyl)-4-phenyl-N-((4-(trifluoromethyl)phenyl)sulfonyl)-4,5-dihydro-1H-pyrazole-1-carbimidoyl chloride (0.23 g, 0.43 mmol, 9.0). After stirring at rt for 18 hours, the reaction was diluted with dichloromethane (20 mL) and washed with aqueous ammonium chloride solution (15 mL). The organic layer was dried over magnesium sulfate, filtered, and concentrated in vacuo. The residue was purified by flash chromatography on silica eluting with heptane/ethyl acetate mixtures (100/0 to 60/40, v/v) to give a colorless oil as a racemic mixture which was further subjected to SFC separation conditions.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 2.5 ml/min of CO₂ (60%)—MeOH+0.1% diethylamine, at 35° C., BPR 100 bar. Acquisition frequency was set to 220 nm for the DAD detector. Absolute stereochemistry was not determined. Diastereomers with (-) OR were arbitrary assigned as S isomer.

Compound 17: (22.1 mg, 0.035 mmol, 8% yield, 95% purity, 99% ee, OR: −96.0). ¹H NMR (400 MHz, CDCl₃) δ 8.04 (d, J=8.2 Hz, 2H), 7.63-7.73 (m, 3H), 7.54 (d, J=8.6 Hz, 2H), 7.28-7.35 (m, 3H), 7.23-7.27 (m, 2H), 7.09-7.15 (m, 2H), 4.92 (br s, 2H), 4.69 (dd, J=11.4, 5.4 Hz, 1H), 4.54 (t, J=11.5 Hz, 1H), 3.93-4.14 (m, 3H), 3.46-3.57 (m, 1H), 1.47 ppm (d, J=7.0 Hz, 3H). LC-MS (ESI+): 628.1127 (M+H⁺).

Compound 18: (14.3 mg, 0.022 mmol, 5% yield, 99% purity, 99% ee, OR: −90.7). ¹H NMR (400 MHz, CDCl₃) δ 8.04 (d, J=8.1 Hz, 2H), 7.63-7.73 (m, 3H), 7.51-7.58 (m, 2H), 7.27-7.36 (m, 3H), 7.23-7.27 (m, 2H), 7.12 (dd, J=7.9, 1.3 Hz, 2H), 4.88 (br s, 2H), 4.70 (dd, J=11.3, 5.4 Hz, 1H), 4.52 (t, J=11.5 Hz, 1H), 4.04-4.13 (m, 2H), 3.93-4.03 (m, 1H), 3.45-3.56 (m, 1H), 1.47 ppm (d, J=7.0 Hz, 3H). LC-MS (ESI+): 628.0 (M+H⁺).

Compound 19: (25.8 mg, 0.04 mmol, 10% yield, 99% purity, 99% ee, OR: +81.8). ¹H NMR (400 MHz, CDCl₃) δ 8.04 (d, J=8.1 Hz, 2H), 7.68 (br d, J=8.3 Hz, 3H), 7.54 (d, J=8.7 Hz, 2H), 7.27-7.35 (m, 3H), 7.23-7.27 (m, 2H), 7.09-7.14 (m, 2H), 4.98 (br s, 2H), 4.70 (dd, J=11.4, 5.5 Hz, 1H), 4.52 (t, J=11.5 Hz, 1H), 4.04-4.13 (m, 2H), 3.92-4.02 (m, 1H), 3.46-3.55 (m, 1H), 1.46 ppm (d, J=7.0 Hz, 3H). LC-MS (ESI+): 628.0 (M+H⁺).

Compound 20: (17.7 mg, 0.03 mmol, 7% yield, 99% purity, 99% ee, OR: +102.7). ¹H NMR (400 MHz, CDCl₃) δ 8.04 (d, J=8.1 Hz, 2H), 7.68 (d, J=8.2 Hz, 3H), 7.6 (m, 2H), 7.4 (m, 4H), 7.3 (m, 1H), 7.2 (m, 2H), 4.85 (br s, 2H), 4.70 (dd, J=5.3, 11.3 Hz, 1H), 4.54 (t, J=11.5 Hz, 1H), 3.9-4.1 (m, 3H), 3.5 (m, 1H), 1.48 (d, J=7.0 Hz, 3H). LC-MS (ESI+): 628.0 (M+H⁺).

### Synthesis of (S)-3-(4-chlorophenyl)-N′-((4-ethynylphenyl)sulfonyl)-4-phenyl-N-(2-sulfamoylethyl)-4,5-dihydro-1H-pyrazole-1-carboximidamide (Compound 21) & (R)-3-(4-chlorophenyl)-N′-((4-ethynylphenyl)sulfonyl)-4-phenyl-N-(2-sulfamoylethyl)-4,5-dihydro-1H-pyrazole-1-carboximidamide (Compound 22)

### Synthesis of Compound 22.1

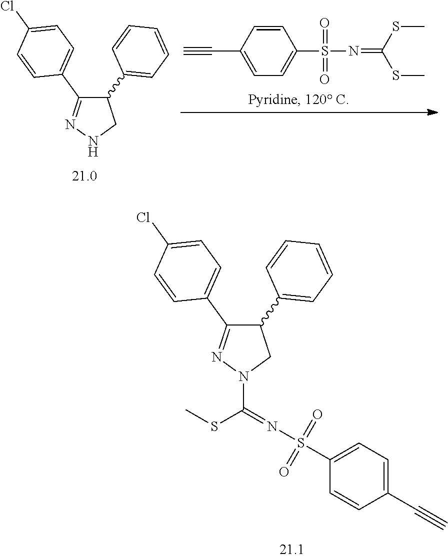

Dimethyl ((4-ethynylphenyl)sulfonyl)carbonimidodithioate (0.43 g, 1.5 mmol, 1 eq.) and 3-(4-chlorophenyl)-4-phenyl-4,5-dihydro-1H-pyrazole (0.39 g, 1.5 mmol, 21.0, CAS #59074-26-9) were mixed in pyridine (7.5 mL). After stirring at 120° C. for 18 h, the reaction mixture was concentrated to dryness and the crude purified by column chromatography eluting with ethyl acetate/heptane mixtures (0/100 to 30/70) to get the expected product as a yellowish oil (0.35 g, 0.71 mmol, 47% yield). LC-MS (ESI+): 494.1 (M+H⁺).

### Synthesis of Compounds 21 and 22

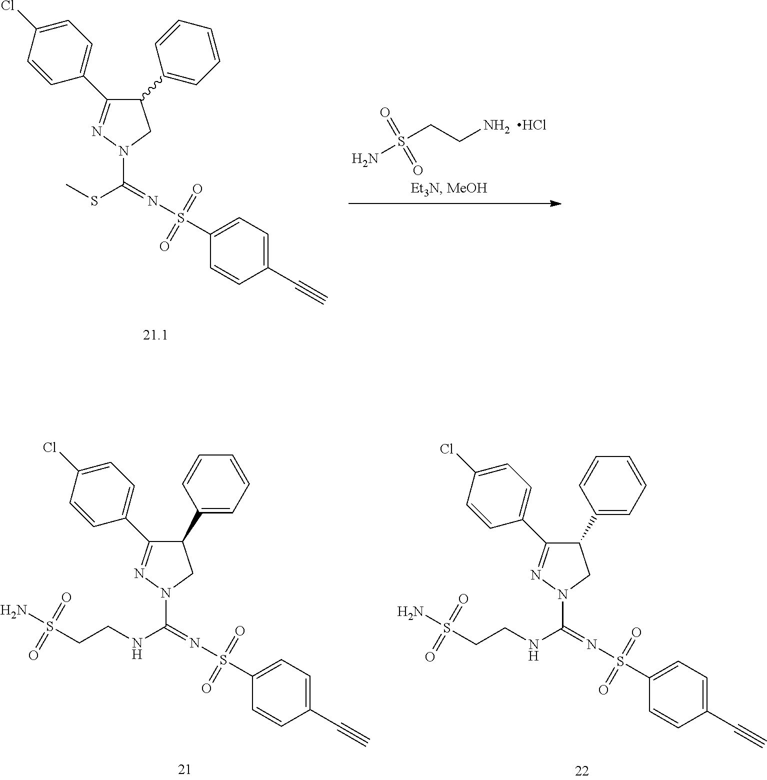

2-aminoethane-1-sulfonamide hydrochloride (230 mg, 1.44 mmol, 2 eq.) was added to a stirred solution of methyl 3-(4-chlorophenyl)-N-((4-ethynylphenyl)sulfonyl)-4-phenyl-4,5-dihydro-1H-pyrazole-1-carbimidothioate (355 mg, 0.72 mmol, 21.1) and triethylamine (0.3 mL, 2.16 mmol, 3 eq.) in methanol (3.6 mL). After stirring at room temperature for 18 hours, solvents were removed under reduced pressure and the crude was chromatographed on silica eluting with ethyl acetate/heptane mixtures (0/100 to 60/40). The desired fractions were collected and repurified by flash chromatography on silica eluting with ethyl acetate/heptane mixtures (0/100 to 50/50, v/v) to give a colorless oil, which was further subjected to SFC purification to resolve the two enantiomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—MeOH+0.1% diethylamine, at 35° C., BPR 100 bar. Acquisition frequency was set to 220 nm for the DAD detector.

Compound 21: (63.6 mg, 0.11 mmol, 16% yield, 99% purity, 99% ee, OR: −85.6). ¹H NMR (DMSO-d₆, 400 MHz) δ 8.18 (br s, 1H), 7.81 (d, J=8.4 Hz, 2H), 7.72 (d, J=8.6 Hz, 2H), 7.57 (d, J=8.4 Hz, 2H), 7.45 (d, J=8.6 Hz, 2H), 7.3-7.4 (m, 2H), 7.2-7.3 (m, 3H), 7.03 (s, 2H), 5.09 (dd, J=4.3, 11.2 Hz, 1H), 4.53 (t, J=11.2 Hz, 1H), 4.39 (s, 1H), 4.08 (br dd, J=4.3, 11.1 Hz, 1H), 3.7-3.8 (m, 2H), 3.3 (m, 2H). LC-MS (ESI+): 570.0 (M+H⁺).

Compound 22: (56.0 mg, 0.1 mmol, 14% yield, 99% purity, 99% ee, OR: +120.1). 1H NMR (DMSO-d₆, 400 MHz) δ 8.1-8.2 (m, 1H), 7.81 (d, J=8.4 Hz, 2H), 7.72 (d, J=8.6 Hz, 2H), 7.57 (d, J=8.4 Hz, 2H), 7.45 (d, J=8.6 Hz, 2H), 7.3-7.4 (m, 2H), 7.2-7.3 (m, 3H), 7.02 (s, 2H), 5.09 (dd, J=4.4, 11.2 Hz, 1H), 4.53 (t, J=11.2 Hz, 1H), 4.39 (s, 1H), 4.08 (br dd, J=4.3, 11.2 Hz, 1H), 3.73 (q, J=6.2 Hz, 2H), 3.2-3.3 (m, 2H). LC-MS (ESI+): 570.0 (M+H⁺).

### Synthesis of (S,E)-3-(4-chlorophenyl)-4-phenyl-N-((R)-1-sulfamoylpropan-2-yl)-N′-((4-(trifluoromethyl)phenyl)sulfonyl)-4,5-dihydro-1H-pyrazole-1-carboximidamide (Compound 23) & (R,E)-3-(4-chlorophenyl)-4-phenyl-N-((R)-1-sulfamoylpropan-2-yl)-N′-((4-(trifluoromethyl)phenyl)sulfonyl)-4,5-dihydro-1H-pyrazole-1-carboximidamide (Compound 24)

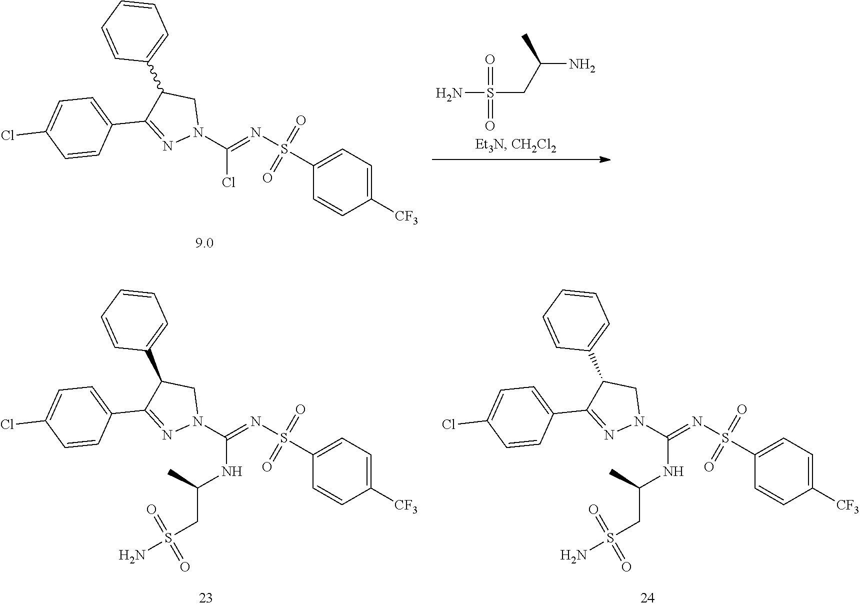

To a solution of (R)-2-aminopropane-1-sulfonamide (120 mg, 0.86 mmol, 1.5 eq.) and triethylamine (0.24 mL, 1.7 mmol, 3.0 eq.) in dichloromethane (5 mL) was added (Z)-3-(4-chlorophenyl)-4-phenyl-N-((4-(trifluoromethyl)phenyl)sulfonyl)-4,5-dihydro-1H-pyrazole-1-carbimidoyl chloride (300 mg, 0.57 mmol, 9.0). After stirring at room temperature for 18 hours, the reaction was concentrated in vacuo and the crude was purified by flash chromatography on silica eluting with ethyl acetate/heptane mixtures (0/100 to 60/40, v/v) to give a colorless oil, which was further subjected to SFC separation, giving the following 2 different isomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%)—MeOH+0.1% diethylamine, at 35° C., BPR 100 bar. Acquisition frequency was set to 220 nm for the DAD detector.

Compound 23: (30.0 mg, 0.05 mol, 8% yield, 99% purity, 99% ee, OR: −71.4). ¹H NMR (DMSO-d₆, 400 MHz) δ 8.03 (br d, J=8.2 Hz, 3H), 7.86 (d, J=8.3 Hz, 2H), 7.72 (d, J=8.5 Hz, 2H), 7.45 (d, J=8.6 Hz, 2H), 7.3-7.4 (m, 2H), 7.2-7.3 (m, 3H), 6.96 (br s, 2H), 5.10 (dd, J=4.2, 11.2 Hz, 1H), 4.59 (br t, J=11.2 Hz, 1H), 4.39 (br s, 1H), 4.08 (br dd, J=4.1, 11.1 Hz, 1H), 3.45 (dd, J=6.3, 14.1 Hz, 1H), 3.2-3.3 (m, 1H), 1.25 (d, J=6.7 Hz, 3H). LC-MS (ESI+): 628.0 (M+H⁺).

Compound 24: (16.4 mg, 0.026 mmol, 4% yield, 99% purity, 99% ee, OR: +53.2). ¹H NMR (DMSO-d₆, 400 MHz) δ 8.03 (br d, J=8.2 Hz, 3H), 7.85 (d, J=8.3 Hz, 2H), 7.70 (d, J=8.5 Hz, 2H), 7.44 (d, J=8.7 Hz, 2H), 7.3-7.4 (m, 2H), 7.2-7.3 (m, 3H), 6.93 (s, 2H), 5.11 (dd, J=4.2, 11.2 Hz, 1H), 4.54 (t, J=11.1 Hz, 1H), 4.40 (br d, J=6.0 Hz, 1H), 4.11 (br dd, J=4.4, 11.0 Hz, 1H), 3.4 (m, 1H), 3.21 (br dd, J=6.2, 14.1 Hz, 1H), 1.27 (br d, J=6.7 Hz, 3H). LC-MS (ESI+): 628.0 (M+H⁺).

### Synthesis of (S,E)-3-(4-chlorophenyl)-4-phenyl-N-((S)-1-sulfamoylpropan-2-yl)-N′-((4-(trifluoromethyl)phenyl)sulfonyl)-4,5-dihydro-1H-pyrazole-1-carboximidamide (Compound 25) & (R,E)-3-(4-chlorophenyl)-4-phenyl-N-((S)-1-sulfamoylpropan-2-yl)-N′-((4-(trifluoromethyl)phenyl)sulfonyl)-4,5-dihydro-1H-pyrazole-1-carboximidamide (Compound 26)

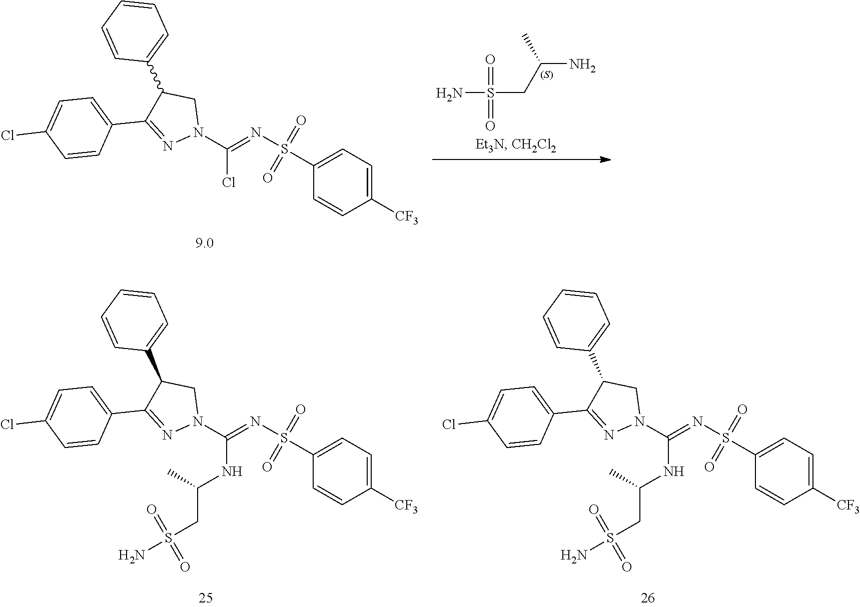

To a solution of (S)-2-aminopropane-1-sulfonamide (100 mg, 0.71 mmol, 1.5 eq.) and triethylamine (0.2 mL, 1.42 mmol, 3.0 eq.) in dichloromethane (2.5 mL) was added (Z)-3-(4-chlorophenyl)-4-phenyl-N-((4-(trifluoromethyl)phenyl)sulfonyl)-4,5-dihydro-1H-pyrazole-1-carbimidoyl chloride (250 mg, 0.47 mmol, 9.0). After stirring at room temperature for 18 hours, solvents were removed under reduced pressure and the residue was purified by flash chromatography on silica eluting with ethyl acetate/heptane mixtures (0/100 to 60/40, v/v) to give a colorless oil which was further subjected to SFC purification to resolve the two diastereoisomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 25: (13.2 mg, 0.02 mol, 4% yield, 99% purity, 99% de, OR: +202.1). ¹H NMR (DMSO-d₆, 400 MHz) δ 8.0-8.1 (m, 3H), 7.86 (d, J=8.4 Hz, 2H), 7.72 (d, J=8.5 Hz, 2H), 7.45 (d, J=8.5 Hz, 2H), 7.3-7.4 (m, 2H), 7.2-7.3 (m, 3H), 6.96 (s, 2H), 5.10 (dd, J=4.2, 11.1 Hz, 1H), 4.59 (t, J=11.2 Hz, 1H), 4.39 (br d, J=5.1 Hz, 1H), 4.08 (br dd, J=4.1, 11.1 Hz, 1H), 3.45 (dd, J=6.3, 14.1 Hz, 1H), 3.24 (dd, J=6.1, 14.0 Hz, 1H), 1.25 (d, J=6.7 Hz, 3H). LC-MS (ESI+): 628.0 (M+H⁺).

Compound 26: (16.1 mg, 0.026 mmol, 5% yield, 92% purity, 99% de, OR: −63.4). ¹H NMR (DMSO-d₆, 400 MHz) δ 8.03 (br d, J=8.2 Hz, 3H), 7.85 (d, J=8.3 Hz, 2H), 7.70 (d, J=8.6 Hz, 2H), 7.44 (d, J=8.6 Hz, 2H), 7.32 (br d, J=7.2 Hz, 2H), 7.2-7.3 (m, 3H), 6.94 (s, 2H), 5.11 (dd, J=4.1, 11.2 Hz, 1H), 4.54 (t, J=11.1 Hz, 1H), 4.39 (br s, 1H), 4.11 (br dd, J=3.9, 10.9 Hz, 1H), 3.4 (m, 1H), 3.2-3.3 (m, 1H), 1.27 (d, J=6.7 Hz, 3H). LC-MS (ESI+): 628.1 (M+H⁺).

### Synthesis of (S)-3-(4-chlorophenyl)-4-phenyl-N-(2-sulfamoylethyl)-N′-((3-(trifluoromethyl)phenyl)sulfonyl)-4,5-dihydro-1H-pyrazole-1-carboximidamide (Compound 27) & (R)-3-(4-chlorophenyl)-4-phenyl-N-(2-sulfamoylethyl)-N′-((3-(trifluoromethyl)phenyl)sulfonyl)-4,5-dihydro-1H-pyrazole-1-carboximidamide (Compound 28)

### Synthesis of Compound 27.1

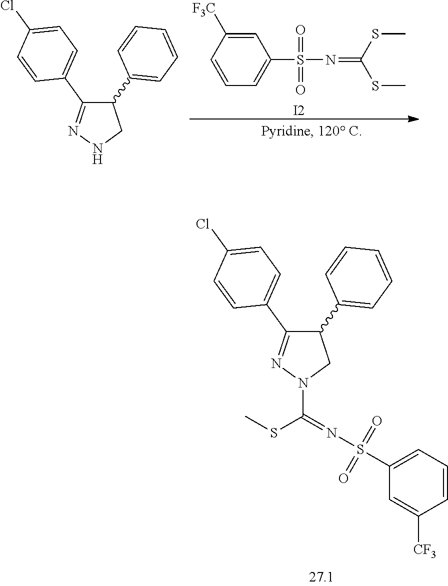

N-(Bis-methylsulfanyl-methylene)-3-trifluoromethyl-benzenesulfonamide (0.54 g, 1.63 mmol, 12 and 3-(4-chlorophenyl)-4-phenyl-4,5-dihydro-1H-pyrazole (0.42 g, 1.63 mmol, 1 eq.) in pyridine (5 mL) was refluxed (120° C.) for 18 h. Upon completion, the reaction mixture was concentrated to dryness and the crude purified by column chromatography eluting with ethyl acetate/heptane mixtures (0/100 to 30/70, v/v) to yield the expected product as a yellow foam (0.6 g, 1.12 mmol, 69% yield). LC-MS (ESI+): 538.1 (M+H⁺).

### Synthesis of Compounds 27 and 28

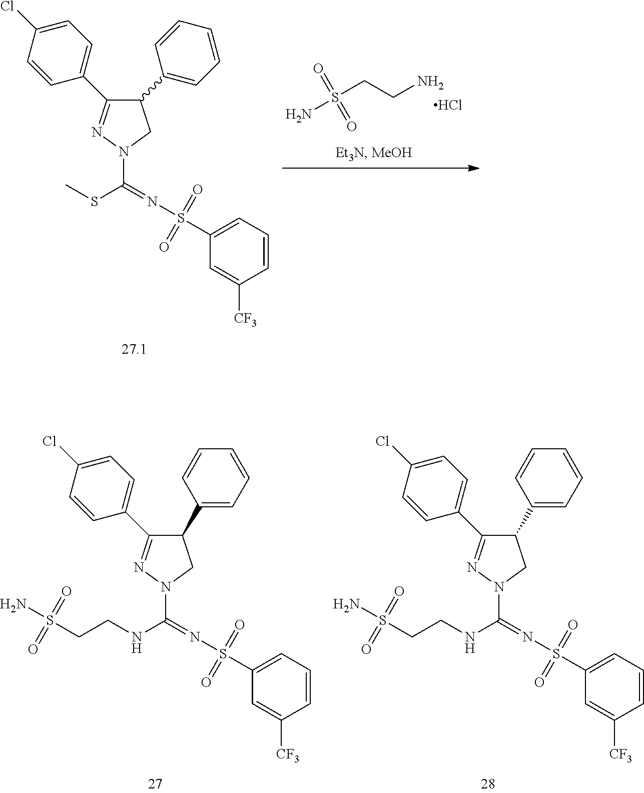 [SMILES: CSC(=NS(=O)(=O)c1cccc(C(F)(F)F)c1)N1CC(c2ccccc2)C(c2ccc(Cl)cc2)=N1 ; NCCS(N)(=O)=O ; Cl ; NS(=O)(=O)CCNC(=NS(=O)(=O)c1cccc(C(F)(F)F)c1)N1C[C@H](c2ccccc2)C(c2ccc(Cl)cc2)=N1 ; NS(=O)(=O)CCNC(=NS(=O)(=O)c1cccc(C(F)(F)F)c1)N1C[C@@H](c2ccccc2)C(c2ccc(Cl)cc2)=N1]

2-aminoethane-1-sulfonamide hydrochloride (90 mg, 0.56 mmol, 1.2 eq.) was added to a stirring solution of N-{[3-(4-Chloro-phenyl)-4-phenyl-4,5-dihydro-pyrazol-1-yl]-methylsulfanyl-methylene}-3-trifluoromethyl-benzenesulfonamide (250 mg, 0.46 mmol, 27.1) and triethylamine (0.2 mL, 1.4 mmol, 3 eq.) in methanol (4 mL). After stirring at room temperature for 18 hours, the solvents were removed under reduced pressure. The residue was chromatographed on silica eluting with ethyl acetate/heptane mixtures (0/100 to 60/40, v/v) to obtain a colorless oil, which was further subjected to SFC purification to resolve the two enantiomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—iPrOH+0.1% diethylamine, at 35° C., BPR 100 bar. Acquisition frequency was set to 220 nm for the DAD detector.

Compound 27: (65.0 mg, 0.10 mmol, 23% yield, 99% purity, 99% ee, OR: −109.5). ¹H NMR (DMSO-d₆, 400 MHz) δ 8.16 (br s, 1H), 8.08 (br d, J=7.9 Hz, 1H), 7.97 (s, 1H), 7.85 (br d, J=7.8 Hz, 1H), 7.60-7.72 (m, 3H), 7.38 (d, J=8.6 Hz, 2H), 7.23-7.30 (m, 2H), 7.13-7.22 (m, 3H), 6.94 (s, 2H), 5.04 (dd, J=11.2, 4.3 Hz, 1H), 4.48 (t, J=11.2 Hz, 1H), 4.00 (dd, J=11.0, 4.3 Hz, 1H), 3.66 (br d, J=3.5 Hz, 2H), 3.17-3.21 ppm (m, 2H). LC-MS (ESI+): 614.0 (M+H⁺).

Compound 28: (66.3 mg, 0.11 mmol, 23% yield, 99% purity, 99% ee, OR: +86.7). ¹H NMR (DMSO-d₆, 400 MHz) δ 8.22 (br s, 1H), 8.15 (br d, J=7.9 Hz, 1H), 8.04 (s, 1H), 7.92 (br d, J=7.8 Hz, 1H), 7.67-7.78 (m, 3H), 7.45 (d, J=8.6 Hz, 2H), 7.30-7.36 (m, 2H), 7.20-7.29 (m, 3H), 7.00 (br s, 2H), 5.10 (dd, J=11.1, 4.3 Hz, 1H), 4.54 (t, J=11.2 Hz, 1H), 4.07 (dd, J=11.0, 4.3 Hz, 1H), 3.72 (br s, 2H), 3.23-3.28 ppm (m, 2H). LC-MS (ESI+): 614.0 (M+H⁺).

### Synthesis of (S)-3-(4-chlorophenyl)-N′-((2-chlorophenyl)sulfonyl)-4-phenyl-N-(2-sulfamoylethyl)-4,5-dihydro-1H-pyrazole-1-carboximidamide (Compound 29) & (R)-3-(4-chlorophenyl)-N′-((2-chlorophenyl)sulfonyl)-4-phenyl-N-(2-sulfamoylethyl)-4,5-dihydro-1H-pyrazole-1-carboximidamide (Compound 30)

### Synthesis of 29.1

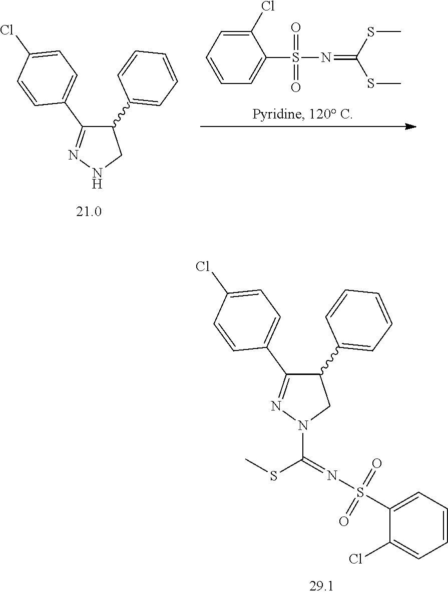

N-(Bis-methylsulfanyl-methylene)-2-chloro-benzenesulfonamide (0.5 g, 1.69 mmol, 13) and 3-(4-chlorophenyl)-4-phenyl-4,5-dihydro-1H-pyrazole (0.42 g, 1.63 mmol, 21.0) in pyridine (6 mL) were refluxed (120° C.) for 18 h. Upon completion, the reaction mixture was concentrated to dryness and the residue was purified by column chromatography eluting with ethyl acetate/heptane mixtures (0/100 to 40/60, v/v) to yield the desired product as a yellow foam (0.58 g, 1.15 mmol, 71% yield). LC-MS (ESI+): 504.0 (M+H⁺).

### Synthesis of Compounds 29 and 30

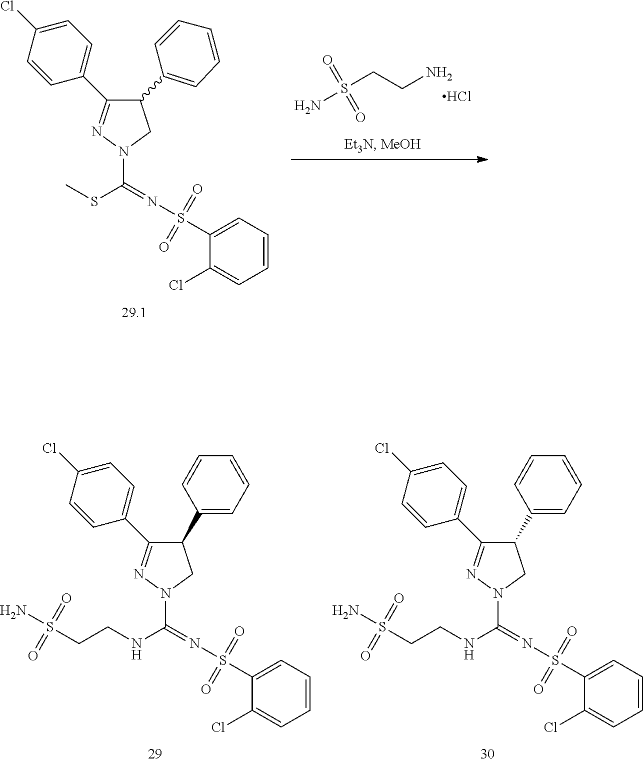 [SMILES: CSC(=NS(=O)(=O)c1ccccc1Cl)N1CC(c2ccccc2)C(c2ccc(Cl)cc2)=N1 ; NCCS(N)(=O)=O ; Cl ; NS(=O)(=O)CCNC(=NS(=O)(=O)c1ccccc1Cl)N1C[C@H](c2ccccc2)C(c2ccc(Cl)cc2)=N1 ; NS(=O)(=O)CCNC(=NS(=O)(=O)c1ccccc1Cl)N1C[C@@H](c2ccccc2)C(c2ccc(Cl)cc2)=N1]

2-Aminoethane-1-sulfonamide hydrochloride (368 mg, 2.29 mmol, 2 eq.) was added to a stirring solution of Methyl 3-(4-chlorophenyl)-N-((2-chlorophenyl)sulfonyl)-4-phenyl-4,5-dihydro-1H-pyrazole-1-carbimidothioate (578 mg, 1.15 mmol, 29.1) and triethylamine (0.5 mL, 3.4 mmol, 3 eq.) in methanol (4 mL). After stirring at rt for 18 hr, solvents were removed under reduced pressure and the residue was chromatographed on silica eluting with ethyl acetate/heptane mixtures (0/100 to 80/20, v/v) to obtain a colorless oil, which was further subjected to SFC purification to resolve the two enantiomers. SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—MeOH+0.1% diethylamine, at 35° C., BPR 100 bar. Acquisition frequency was set to 220 nm for the DAD detector.

Compound 29: (61.7 mg, 0.11 mmol, 9% yield, 99% purity, 99% ee, OR: −90.0). ¹H NMR (DMSO-d₆, 400 MHz) δ 8.16 (br t, J=5.1 Hz, 1H), 8.02 (dd, J=1.5, 7.7 Hz, 1H), 7.73 (d, J=8.6 Hz, 2H), 7.5-7.6 (m, 2H), 7.4-7.5 (m, 3H), 7.3-7.4 (m, 2H), 7.2-7.3 (m, 3H), 7.02 (s, 2H), 5.09 (dd, J=4.3, 11.2 Hz, 1H), 4.54 (t, J=11.2 Hz, 1H), 4.0-4.1 (m, 1H), 3.77 (q, J=6.3 Hz, 2H), 3.2-3.3 (m, 2H). LC-MS (ESI+): 580.2 (M+H⁺).

Compound 30: (59.4 mg, 0.10 mmol, 9% yield, 99% purity, 99% ee, OR: +100.6). ¹H NMR (DMSO-d₆, 400 MHz) δ 8.16 (br t, J=5.3 Hz, 1H), 8.02 (dd, J=1.3, 7.7 Hz, 1H), 7.73 (d, J=8.6 Hz, 2H), 7.5-7.6 (m, 2H), 7.4-7.5 (m, 3H), 7.3-7.4 (m, 2H), 7.2-7.3 (m, 3H), 7.02 (s, 2H), 5.09 (dd, J=4.3, 11.2 Hz, 1H), 4.53 (t, J=11.2 Hz, 1H), 4.0-4.1 (m, 1H), 3.77 (q, J=6.4 Hz, 2H), 3.2-3.3 (m, 2H). LC-MS (ESI+): 580.2 (M+H⁺).

### Synthesis of (S)-3-(4-chlorophenyl)-N′-((3-chlorophenyl)sulfonyl)-4-phenyl-N-(2-sulfamoylethyl)-4,5-dihydro-1H-pyrazole-1-carboximidamide (Compound 31) & (S)-3-(4-chlorophenyl)-N′-((3-chlorophenyl)sulfonyl)-4-phenyl-N-(2-sulfamoylethyl)-4,5-dihydro-1H-pyrazole-1-carboximidamide (Compound 32)

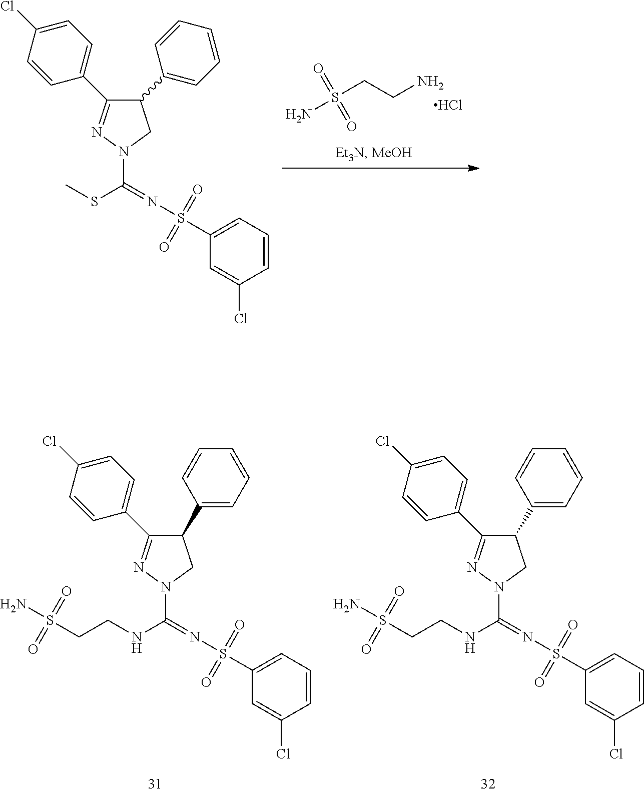 [SMILES: CSC(=NS(=O)(=O)c1cccc(Cl)c1)N1CC(c2ccccc2)C(c2ccc(Cl)cc2)=N1 ; NCCS(N)(=O)=O ; Cl ; NS(=O)(=O)CCNC(=NS(=O)(=O)c1cccc(Cl)c1)N1C[C@H](c2ccccc2)C(c2ccc(Cl)cc2)=N1 ; NS(=O)(=O)CCNC(=NS(=O)(=O)c1cccc(Cl)c1)N1C[C@@H](c2ccccc2)C(c2ccc(Cl)cc2)=N1]

2-Aminoethane-1-sulfonamide hydrochloride (0.2 g, 1.2 mmol, 1.2 eq.) was added to a stirring solution of methyl 3-(4-chlorophenyl)-N-((3-chlorophenyl)sulfonyl)-4-phenyl-4,5-dihydro-1H-pyrazole-1-carbimidothioate (0.51 g, 1.011 mmol, CAS #656827-47-3) and triethylamine (0.42 mL, 3.03 mmol, 3 eq.) in methanol (5 mL). After stirring at rt for 18 hr, solvents were removed under reduced pressure. The residue was chromatographed on silica eluting with ethyl acetate/heptane mixtures (0/100 to 80/20) to obtain a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 31: (72.3 mg, 0.12 mmol, 12% yield, 99% purity, 99% ee, OR: −99.1). ¹H NMR (DMSO-d₆, 400 MHz) δ 8.19 (br t, J=5.3 Hz, 1H), 7.76-7.81 (m, 2H), 7.71 (d, J=8.6 Hz, 2H), 7.58-7.62 (m, 1H), 7.49-7.55 (m, 1H), 7.45 (d, J=8.6 Hz, 2H), 7.30-7.37 (m, 2H), 7.20-7.28 (m, 3H), 7.01 (s, 2H), 5.09 (dd, J=11.2, 4.4 Hz, 1H), 4.53 (t, J=11.2 Hz, 1H), 3.99-4.10 (m, 1H), 3.69-3.80 (m, 2H), 3.27 (br s, 2H) ppm. LC-MS (ESI+): 580.04 (M+H⁺).

Compound 32: (80.4 mg, 0.14 mmol, 14% yield, 99% purity, 99% ee, OR: +111.9). ¹H NMR (DMSO-d₆, 400 MHz) δ 8.19 (br t, J=5.2 Hz, 1H), 7.77-7.82 (m, 2H), 7.71 (d, J=8.6 Hz, 2H), 7.57-7.62 (m, 1H), 7.49-7.55 (m, 1H), 7.45 (d, J=8.6 Hz, 2H), 7.30-7.36 (m, 2H), 7.20-7.29 (m, 3H), 7.01 (s, 2H), 5.09 (dd, J=11.1, 4.2 Hz, 1H), 4.53 (t, J=11.2 Hz, 1H), 4.06 (dd, J=11.4, 4.6 Hz, 1H), 3.74 (q, J=6.1 Hz, 2H), 3.27 (br s, 2H) ppm. LC-MS (ESI+): 580.04 (M+H⁺).

### Synthesis of (S)-3-(4-chlorophenyl)-N′-((2-ethynylphenyl)sulfonyl)-4-phenyl-N-(2-sulfamoylethyl)-4,5-dihydro-1H-pyrazole-1-carboximidamide (Compound 33) & (R)-3-(4-chlorophenyl)-N′-((2-ethynylphenyl)sulfonyl)-4-phenyl-N-(2-sulfamoylethyl)-4,5-dihydro-1H-pyrazole-1-carboximidamide (Compound 34)

### Synthesis of 33.1

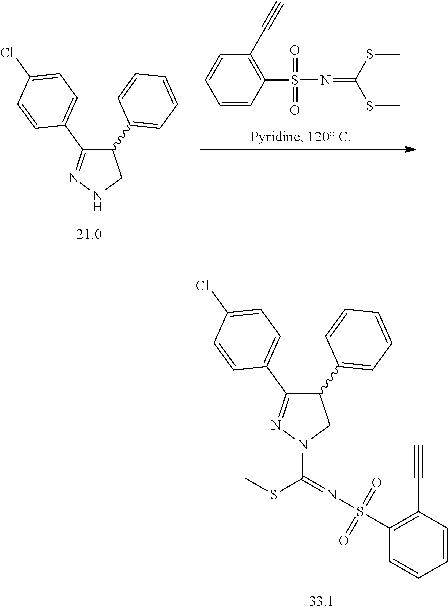

Dimethyl ((2-ethynylphenyl)sulfonyl)carbonimidodithioate (0.47 g, 1.63 mmol, 14) and 3-(4-chlorophenyl)-4-phenyl-4,5-dihydro-1H-pyrazole (0.42 g, 1.63 mmol, 21.0) in pyridine (3 mL) was heated at 120° C. for 18 h. Upon completion, the reaction mixture was concentrated in vacuo and the residue was purified by flash chromatography on silica eluting with ethyl acetate/heptane mixtures (0/100 to 50/50) to afford the desired product as a yellow solid (0.24 g, 0.49 mmol, 30% yield). LC-MS (ESI+): 494.0 (M+H⁺).

### Synthesis of Compounds 33 and 34

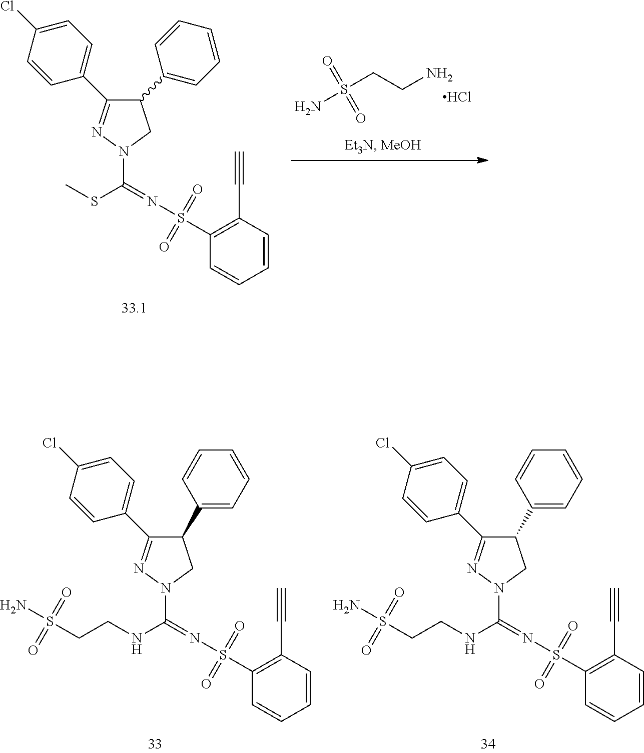 [SMILES: C#Cc1ccccc1S(=O)(=O)N=C(SC)N1CC(c2ccccc2)C(c2ccc(Cl)cc2)=N1 ; NCCS(N)(=O)=O ; Cl ; C#Cc1ccccc1S(=O)(=O)N=C(NCCS(N)(=O)=O)N1C[C@H](c2ccccc2)C(c2ccc(Cl)cc2)=N1 ; C#Cc1ccccc1S(=O)(=O)N=C(NCCS(N)(=O)=O)N1C[C@@H](c2ccccc2)C(c2ccc(Cl)cc2)=N1]

2-aminoethane-1-sulfonamide hydrochloride (0.31 g, 1.95 mmol, 4 eq.) was added to a stirring solution of methyl 3-(4-chlorophenyl)-N-((2-ethynylphenyl)sulfonyl)-4-phenyl-4,5-dihydro-1H-pyrazole-1-carbimidothioate (0.24 g, 0.5 mmol, 33.1) and triethylamine (0.4 mL, 2.9 mmol, 6 eq.) in methanol (5 mL). After stirring at rt for 18 hr, solvents were removed under reduced pressure and the residue was chromatographed on silica eluting with methanol/dichloromethane mixtures (0/100 to 10/90) to give a colorless oil, which was further subjected to SFC purification to resolve the two enantiomers as white solids.

SFC purification was performed on a Jasco 4000 Parallel SFC System (4×Channels 3×UV+1×DAD) using an Phenomenex column (LuxAmylose-1) 150 mm long×4.6 mm I.D. 5 μm particle size, on isocratic mode at 3 ml/min of CO2 (40%)—iPrOH+0.1% diethylamine, at 35° C., BPR 100 bar.

Acquisition frequency was set to 220 nm for the DAD detector.

Compound 33: (52.2 mg, 0.09 mmol, 19% yield, 95% purity, 99% ee, OR: −61.9). ¹H NMR (DMSO-d₆, 400 MHz) δ 8.06 (br s, 1H), 7.91-7.96 (m, 1H), 7.73 (d, J=8.7 Hz, 2H), 7.58-7.63 (m, 1H), 7.47-7.54 (m, 2H), 7.45 (d, J=8.7 Hz, 2H), 7.30-7.36 (m, 2H), 7.21-7.28 (m, 3H), 7.02 (br s, 2H), 5.07 (br dd, J=11.2, 4.5 Hz, 1H), 4.53 (br t, J=11.3 Hz, 1H), 4.47 (s, 1H), 4.01-4.08 (m, 1H), 3.81 (br s, 2H), 3.28-3.30 (m, 2H) ppm. LC-MS (ESI+): 570.9 (M+H⁺).

Compound 34: (56.6 mg, 0.10 mmol, 20% yield, 96% purity, 99% ee, OR: +89.9). ¹H NMR (DMSO-d₆, 400 MHz) δ 8.06 (br s, 1H), 7.91-7.96 (m, 1H), 7.73 (d, J=8.7 Hz, 2H), 7.58-7.64 (m, 1H), 7.47-7.55 (m, 2H), 7.45 (d, J=8.7 Hz, 2H), 7.30-7.35 (m, 2H), 7.22-7.28 (m, 3H), 7.02 (br s, 2H), 5.07 (br dd, J=11.3, 4.4 Hz, 1H), 4.53 (br t, J=11.3 Hz, 1H), 4.47 (s, 1H), 4.05 (br dd, J=11.1, 4.3 Hz, 1H), 3.81 (br s, 2H), 3.28-3.30 (m, 2H) ppm. LC-MS (ESI+): 570.9 (M+H⁺).

### Synthesis of (S)-3-(4-chlorophenyl)-N′-((3-ethynylphenyl)sulfonyl)-4-phenyl-N-(2-sulfamoylethyl)-4,5-dihydro-1H-pyrazole-1-carboximidamide (Compound 35) & (R)-3-(4-chlorophenyl)-N′-((3-ethynylphenyl)sulfonyl)-4-phenyl-N-(2-sulfamoylethyl)-4,5-dihydro-1H-pyrazole-1-carboximidamide (Compound 36)

### Synthesis of 35.1

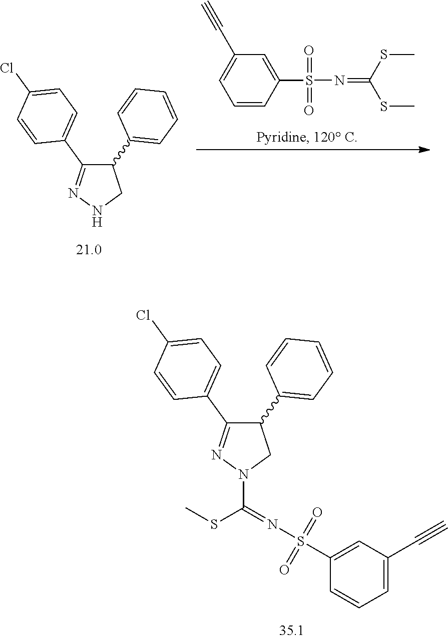 [SMILES: Clc1ccc(C2=NNCC2c2ccccc2)cc1 ; C#Cc1cccc(S(=O)(=O)N=C(SC)SC)c1 ; C#Cc1cccc(S(=O)(=O)N=C(SC)N2CC(c3ccccc3)C(c3ccc(Cl)cc3)=N2)c1]

Dimethyl ((3-ethynylphenyl)sulfonyl)carbonimidodithioate (2.65 g, 9.28 mmol, 15) and 3-(4-chlorophenyl)-4-phenyl-4,5-dihydro-1H-pyrazole (2.4 g, 9.28 mmol, 21.0) in pyridine (18 mL) was heated at 120° C. for 18 h. Upon completion, the reaction mixture was concentrated in vacuo and the residue was purified by flash chromatography on silica eluting with ethyl acetate/heptane mixtures (0/100 to 50/50) to afford the desired product as a yellow solid (1.94 g, 3.94 mmol, 42% yield). LC-MS (ESI+): 494.0 (M+H⁺).

### Synthesis of Compounds 35 and 36

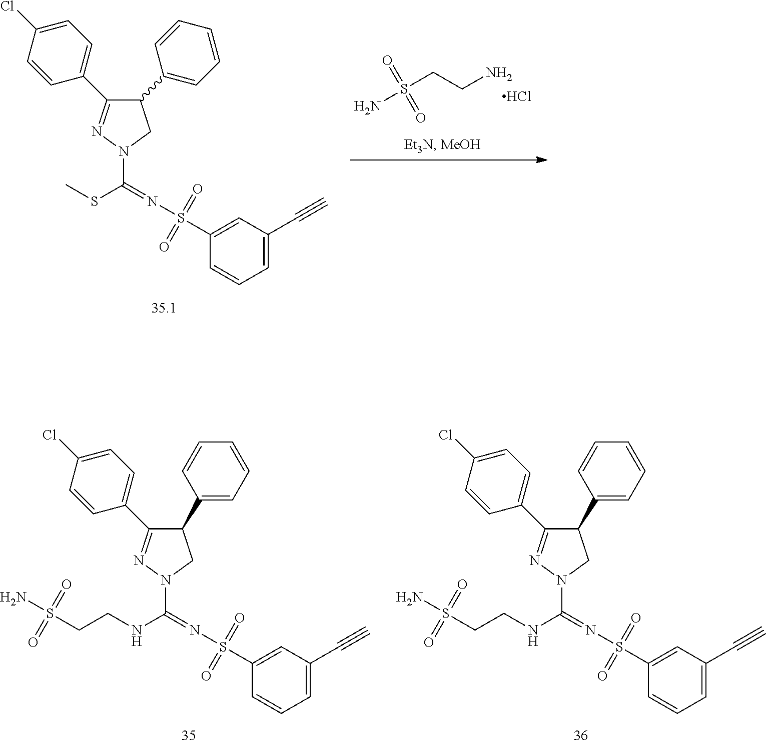 [SMILES: C#Cc1cccc(S(=O)(=O)N=C(SC)N2CC(c3ccccc3)C(c3ccc(Cl)cc3)=N2)c1 ; NCCS(N)(=O)=O ; Cl ; C#Cc1cccc(S(=O)(=O)N=C(NCCS(N)(=O)=O)N2C[C@H](c3ccccc3)C(c3ccc(Cl)cc3)=N2)c1 ; C#Cc1cccc(S(=O)(=O)N=C(NCCS(N)(=O)=O)N2C[C@H](c3ccccc3)C(c3ccc(Cl)cc3)=N2)c1]

2-aminoethane-1-sulfonamide hydrochloride (0.37 g, 2.3 mmol, 4 eq.) was added to a stirring solution of methyl 3-(4-chlorophenyl)-N-((3-ethynylphenyl)sulfonyl)-4-phenyl-4,5-dihydro-1H-pyrazole-1-carbimidothioate (0.28 g, 0.6 mmol, 1 eq.) and triethylamine (0.48 mL, 3.4 mmol, 6 eq.) in methanol (5 mL). After stirring at rt for 18 hr, solvents were removed under reduced pressure and the residue was chromatographed on silica eluting with methanol/dichloromethane mixtures (0/100 to 10/90) to give a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 35: (38.2 mg, 0.07 mmol, 12% yield, 97% purity, 99% ee, OR: −98.8). ¹H NMR (DMSO-d₆, 400 MHz) δ 7.97-8.44 (m, 1H), 7.78-7.89 (m, 2H), 7.66-7.74 (m, J=8.4 Hz, 2H), 7.57-7.64 (m, 1H), 7.48-7.54 (m, 1H), 7.44 (br d, J=8.6 Hz, 2H), 7.30-7.36 (m, 2H), 7.20-7.28 (m, 3H), 7.01 (br s, 2H), 5.08 (br dd, J=11.2, 4.1 Hz, 1H), 4.52 (br t, J=11.1 Hz, 1H), 4.32 (s, 1H), 4.05 (br dd, J=11.0, 4.1 Hz, 1H), 3.74 (br t, J=6.5 Hz, 2H), 3.27 (br d, J=7.3 Hz, 2H) ppm. LC-MS (ESI+): 570.9 (M+H⁺).

Compound 36: (17.1 mg, 0.03 mmol, 5% yield, 97% purity, 99% ee, OR: +68.0). ¹H NMR (DMSO-d₆, 400 MHz) δ 8.17 (br s, 1H), 7.79-7.90 (m, 2H), 7.70 (br d, J=8.4 Hz, 2H), 7.62 (br d, J=7.7 Hz, 1H), 7.50 (t, J=7.8 Hz, 1H), 7.44 (d, J=8.6 Hz, 2H), 7.30-7.37 (m, 2H), 7.20-7.28 (m, 3H), 7.01 (br s, 2H), 5.08 (br dd, J=11.3, 4.1 Hz, 1H), 4.52 (br t, J=11.2 Hz, 1H), 4.32 (s, 1H), 4.05 (br dd, J=11.3, 3.9 Hz, 1H), 3.74 (br t, J=6.5 Hz, 2H), 3.26 ppm (br s, 2H). LC-MS (ESI+): 570.9 (M+H⁺).

### Synthesis of (S,Z)-3-(4-chlorophenyl)-N′-((2,4-difluorophenyl)sulfonyl)-4-phenyl-N-(2-sulfamoylethyl)-4,5-dihydro-1H-pyrazole-1-carboximidamide (Compound 37) & (R,Z)-3-(4-chlorophenyl)-N′-((2,4-difluorophenyl)sulfonyl)-4-phenyl-N-(2-sulfamoylethyl)-4,5-dihydro-1H-pyrazole-1-carboximidamide (Compound 38)

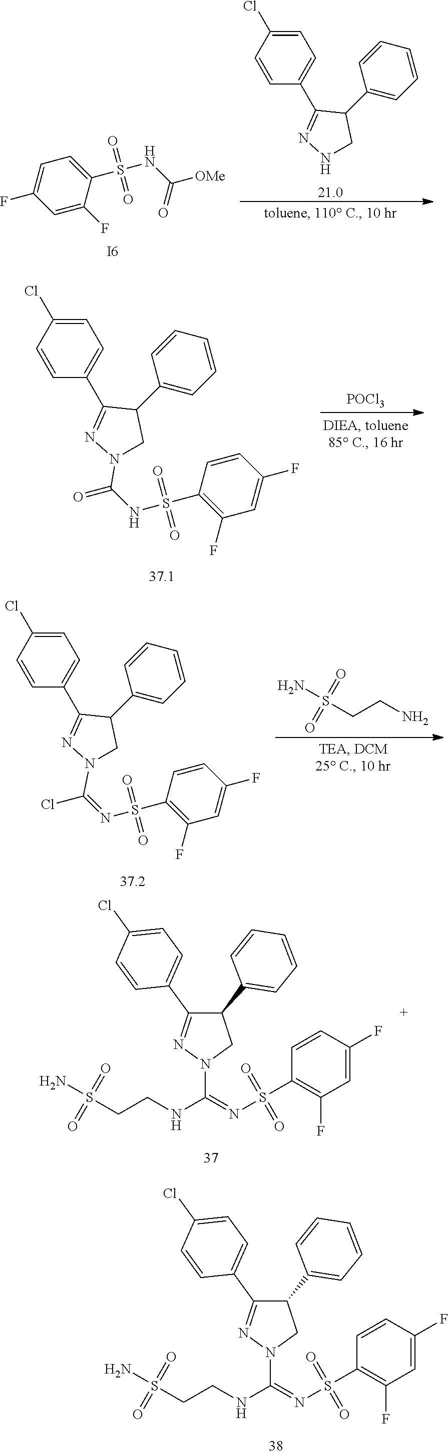

### Synthesis of 37.1

To a solution of 16 (580 mg, 2.26 mmol, 1 eq) in toluene (10 mL) at rt was added methyl N-(2,4-difluorophenyl)sulfonylcarbamate (567.52 mg, 2.26 mmol, 21.0). After stirring at 110° C. for 10 hr, the reaction mixture was cooled to rt and concentrated under reduced pressure to afford a residue, which was triturated with methyl tertiary butyl ether (15 mL) to give 37.1 (900 mg, 1.89 mmol, 84%) as a white solid. LCMS (ESI+): m/z 475.9 (M+H)⁺; ¹H NMR: 400 MHz, DMSO-d₅ δ ppm 3.67 (dd, J=11.27, 4.83 Hz, 1H), 4.27 (t, J=11.38 Hz, 1H), 4.98 (dd, J=11.44, 4.77 Hz, 1H), 7.15-7.37 (m, 6H), 7.43 (d, J=8.70 Hz, 2H), 7.52-7.64 (m, 1H), 7.85 (d, J=8.58 Hz, 2H), 8.02 (td, J=8.64, 6.44 Hz, 1H), 11.83-12.19 (m, 1H)

### Synthesis of 37.2

To a solution of 37.1 (500 mg, 1.05 mmol, 1 eq) in toluene (5 mL) at rt were added N, N-diisopropylethylamine (274.51 μL, 1.58 mmol, 1.5 eq) and phosphorus oxychloride (126.93 μL, 1.37 mmol, 1.3 eq). After stirring at 85° C. for 16 hr, the reaction mixture was concentrated under reduced pressure to get a residue, which was triturated with methyl tertiary butyl ether (10 mL) to give 37.2 (400 mg, 809.16 μmol, 77%) as a yellow solid. The solid was used to the next step directly without further purification.

### Synthesis of Compound 37 and 38

To a solution of 37.2 (390 mg, 788.93 μmol, 1 eq) in dichloromethane (5 mL) at rt were added triethylamine (319.32 mg, 3.16 mmol, 439.24 μL, 4 eq) and 2-aminoethanesulfonamide (146.93 mg, 1.18 mmol, 1.5 eq). After stirring at rt for 12 hr, the reaction mixture was concentrated under reduced pressure to get a solid, which was triturated with methyl tertiary butyl ether (10 mL) to afford racemate (380 mg, 652.87 μmol, 83%) as a white solid. The racemate was separated by SFC to obtain Compound 37 (22 mg, 35.15 μmol, 5%) and Compound 38 (29 mg, 49.82 μmol, 8%) as white solids.

Method of the SFC: Column: DAICEL CHIRALPAK AD (250 mm*30 mm, 10 μm); Mobile phase A: CO₂; Mobile phase B: IPA (0.1% IPAm, v/v); B %: 55%, Run time: 10 min

Compound 37: ¹H NMR (400 MHz, DMSO-d₆) δ ppm 3.22-3.27 (m, 2H), 3.71 (q, J=6.32 Hz, 2H), 4.06 (dd, J=11.00, 4.40 Hz, 1H), 4.54 (t, J=11.19 Hz, 1H), 5.11 (dd, J=11.31, 4.34 Hz, 1H), 7.01 (s, 2H), 7.15 (td, J=8.44, 2.32 Hz, 1H), 7.21-7.28 (m, 3H), 7.30-7.37 (m, 3H), 7.46 (d, J=8.56 Hz, 2H), 7.71-7.76 (m, 2H), 7.84-7.91 (m, 1H), 8.23 (br t, J=5.50 Hz, 1H); LCMS (ESI+): m/z 582.0 (M+H)⁺; OR: −118.40

Compound 38: ¹H NMR (400 MHz, DMSO-d₆) δ ppm 3.22-3.28 (m, 2H), 3.71 (q, J=6.40 Hz, 2H), 4.06 (dd, J=11.07, 4.34 Hz, 1H), 4.54 (t, J=11.19 Hz, 1H), 5.11 (dd, J=11.19, 4.46 Hz, 1H), 6.96-7.04 (m, 2H), 7.15 (td, J=8.50, 2.08 Hz, 1H), 7.21-7.28 (m, 3H), 7.30-7.39 (m, 3H), 7.43-7.49 (m, 2H), 7.71-7.77 (m, 2H), 7.83-7.92 (m, 1H), 8.23 (br t, J=5.20 Hz, 1H); LCMS (ESI+): m/z 582.0 (M+H)⁺; OR: 116.0.

### Synthesis of (S,Z)-3-(4-chlorophenyl)-N′-((3,5-difluorophenyl)sulfonyl)-4-phenyl-N-(2-sulfamoylethyl)-4,5-dihydro-1H-pyrazole-1-carboximidamide (Compound 39) & (S,Z)-3-(4-chlorophenyl)-N′-((3,5-difluorophenyl)sulfonyl)-4-phenyl-N-(2-sulfamoylethyl)-4,5-dihydro-1H-pyrazole-1-carboximidamide (Compound 40)

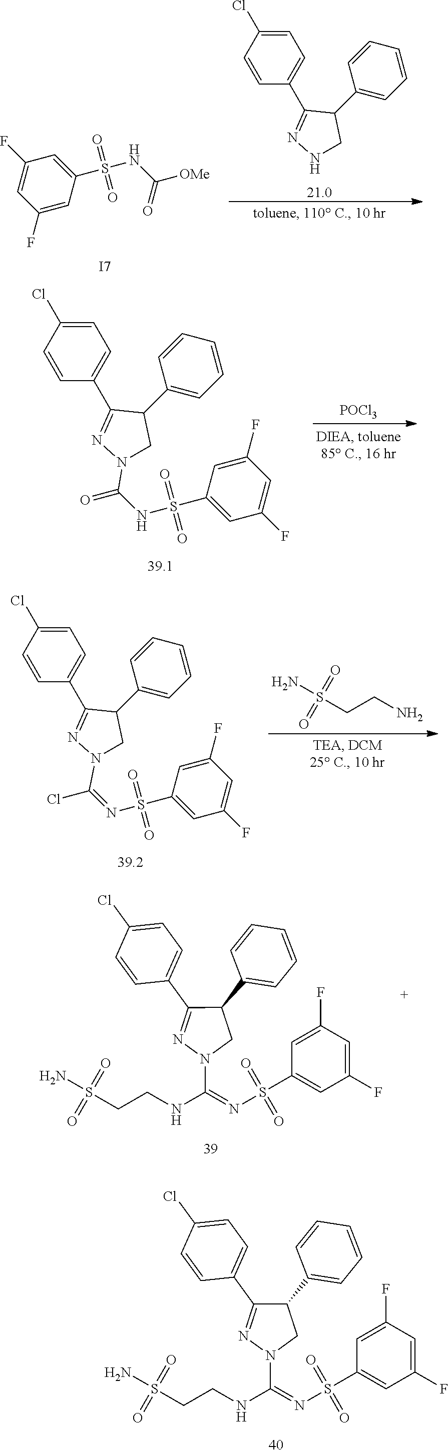

### Synthesis of 39.1

To a solution of 21.0 (1 g, 3.90 mmol, 1 eq) in toluene (10 mL) at rt was added methyl N-(3, 5-difluorophenyl)sulfonylcarbamate (978.49 mg, 3.90 mmol, 17). After stirring at 110° C. for 10 hr, the reaction mixture was cooled down to rt and concentrated under reduced pressure to afford a solid, which was triturated with methyl tertiary butyl ether (15 mL) to give 39.1 (690 mg, 1.30 mmol, 34%) as a white solid. LCMS (ESI+): m/z 475.9 (M+H)⁺;¹H NMR: 400 MHz, DMSO-d₆ 5 ppm 3.70 (dd, J=11.27, 4.95 Hz, 1H), 4.30 (t, J=11.32 Hz, 1H), 4.99 (dd, J=11.44, 4.77 Hz, 1H), 7.12-7.34 (m, 7H), 7.44 (d, J=8.70 Hz, 2H), 7.67 (br d, J=5.36 Hz, 2H), 7.73-7.83 (m, 2H), 11.43-12.04 (m, 1H).

### Synthesis of 39.2

To a solution of 39.1 (500 mg, 1.05 mmol, 1 eq) in toluene (5 mL) at rt were added N, N-diisopropylethylamine (274.51 μL, 1.58 mmol, 1.5 eq) and phosphorus oxychloride (126.93 μL, 1.37 mmol, 1.3 eq). After stirring at 85° C. for 16 hr, the reaction mixture was cooled to rt and concentrated under reduced pressure. The solid was triturated with methyl tertiary butyl ether (10 mL) to give 39.2 (400 mg, 809.16 μmol, 77%) as a yellow solid. LCMS (ESI+): m/z 493.9 (M+H)⁺. ¹H NMR: 400 MHz, DMSO-d₆ δ ppm 4.03 (dd, J=12.59, 5.26 Hz, 1H), 4.68 (t, J=11.80 Hz, 1H), 5.28 (dd, J=10.94, 5.07 Hz, 1H), 7.24-7.36 (m, 6H), 7.47 (d, J=8.56 Hz, 2H), 7.63 (t, J=4.22 Hz, 2H), 7.71 (d, J=8.56 Hz, 2H)

### Synthesis of Compounds 39 and 40

To a solution of 39.2 (300 mg, 606.87 μmol, 1 eq) in dichloromethane (5 mL) at rt were added triethylamine (84.47 μL, 606.87 μmol, 1 eq) and 2-aminoethanesulfonamide (113.03 mg, 910.31 μmol, 1.5 eq). After stirring at rt for 12 hr, the reaction mixture was concentrated under reduced pressure to afford a solid, which was triturated with methyl tertiary butyl ether (10 mL) to a racemate (220 mg, 321.28 μmol, 53%) as a white solid. LCMS (ESI+): m/z 581.9 (M+H)⁺. The enantiomers were separated by SFC to obtain Compound 39 (35 mg, 60.13 μmol, 18%) and Compound 40 (54 mg, 92.78 μmol, 27%) as white solids.

Method of the SFC: Column: DAICEL CHIRALPAK AD (250 mm*30 mm, 10 μm); Mobile phase A: CO₂; Mobile phase B: IPA (0.1% IPAm, v/v); B %: 55%, Run time: 10 min

Compound 39: ¹H NMR (400 MHz, DMSO-d₆) δ ppm 3.23-3.29 (m, 2H), 3.71 (brs, 2H), 4.06 (dd, J=11.13, 4.40 Hz, 1H), 4.54 (t, J=11.19 Hz, 1H), 5.11 (dd, J=11.13, 4.40 Hz, 1H), 7.00 (s, 2H), 7.21-7.28 (m, 3H), 7.30-7.36 (m, 2H), 7.41-7.51 (m, 5H), 7.72 (d, J=8.68 Hz, 2H), 8.25 (br d, J=1.22 Hz, 1H). LCMS (ESI+): m/z 582.0 (M+H)⁺ OR: −124.60.

Compound 40: ¹H NMR (400 MHz, DMSO-d₆) δ ppm 3.23-3.29 (m, 2H), 3.72 (brt, J=6.72 Hz, 2H), 4.06 (dd, J=11.07, 4.34 Hz, 1H), 4.53 (t, J=11.13 Hz, 1H), 5.10 (dd, J=11.19, 4.34 Hz, 1H), 7.01 (br s, 2H), 7.21-7.28 (m, 3H), 7.30-7.35 (m, 2H), 7.43-7.50 (m, 5H), 7.71 (d, J=8.56 Hz, 2H), 8.10-8.38 (m, 1H); LCMS (ESI+): m/z 582.10 (M+H)⁺ OR: 118.00.

### Synthesis of (S,Z)-4-(N-((3-(4-chlorophenyl)-4-phenyl-4,5-dihydro-1H-pyrazol-1-yl)((2-sulfamoylethyl)amino)methylene)sulfamoyl)benzamide (Compound 41) & (R,Z)-4-(N-((3-(4-chlorophenyl)-4-phenyl-4,5-dihydro-1H-pyrazol-1-yl)((2-sulfamoylethyl)amino)methylene)sulfamoyl)benzamide (Compound 42)

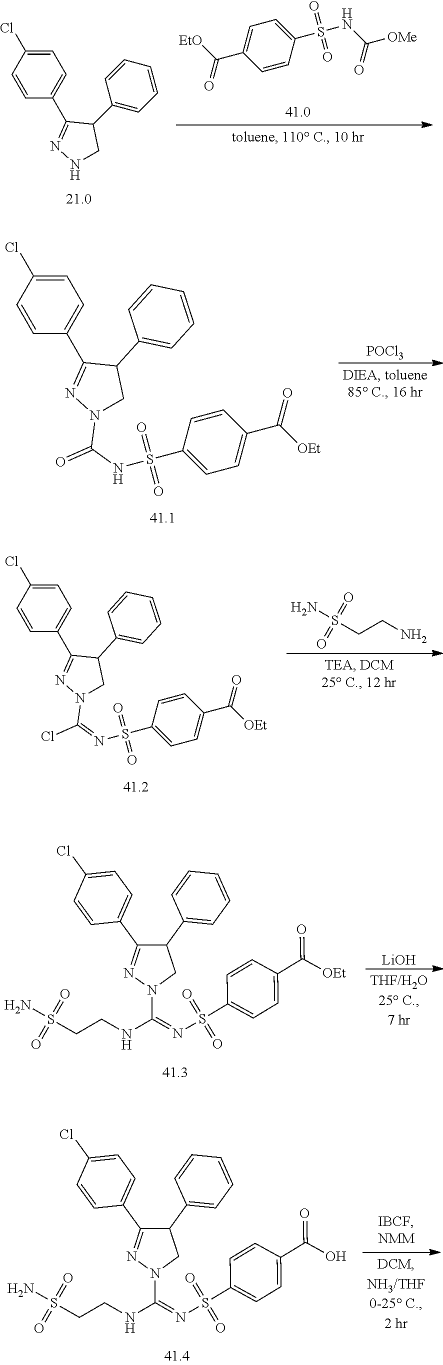

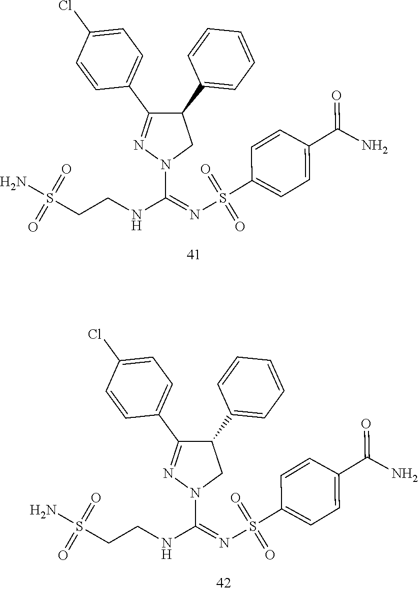 [SMILES: NC(=O)c1ccc(S(=O)(=O)/N=C(/NCCS(N)(=O)=O)N2C[C@H](c3ccccc3)C(c3ccc(Cl)cc3)=N2)cc1 ; NC(=O)c1ccc(S(=O)(=O)/N=C(/NCCS(N)(=O)=O)N2C[C@@H](c3ccccc3)C(c3ccc(Cl)cc3)=N2)cc1]

### Synthesis of 41.1

To a solution of 41.0 (1 g, 3.48 mmol, 1 eq) in toluene (20 mL) at rt was added 21.0 (891.5 mg, 3.48 mmol, 1 eq). After stirring at 110° C. for 10 hr, the reaction mixture was cooled down to rt and concentrated under reduced pressure. The residue was triturated with methyl tertiary butyl ether (10 mL) to give 41.1 (1.5 g, 2.93 mmol, 84%) as a white solid. LCMS (ESI+): m/z 511.9 (M+H)⁺. ¹H NMR: 400 MHz, DMSO-d₆ δ ppm 1.34 (t, J=7.09 Hz, 3H), 3.67 (dd, J=11.31, 4.83 Hz, 1H), 4.27 (t, J=11.37 Hz, 1H), 4.33-4.43 (m, 2H), 4.98 (dd, J=11.43, 4.83 Hz, 1H), 7.15-7.27 (m, 4H), 7.27-7.35 (m, 2H), 7.41-7.48 (m, 2H), 7.81 (d, J=8.56 Hz, 2H), 8.11-8.23 (m, 4H).

### Synthesis of 41.2

To a solution of 41.1 (1.5 g, 2.93 mmol, 1 eq) in toluene (10 mL) at rt were added N, N-diisopropylethylamine (765.45 μL, 4.40 mmol, 1.5 eq) and phosphorus oxychloride (853.95 μL, 3.80 mmol, 1.3 eq). After stirring at 85° C. for 16 hr, the reaction mixture was cooled to rt and concentrated under reduced pressure. The solid was triturated with ethanol (10 mL) to give 41.2 (1 g, 1.89 mmol, 64%) as a white solid, which was used for the next step without further purification. LCMS (ESI+): m/z 529.9 (M+H)⁺.

### Synthesis of 41.3

To a solution of 41.2 (1 g, 1.89 mmol, 1 eq) in dichloromethane (20 mL) at rt were added triethylamine (1.31 mL, 9.45 mmol, 5 eq) and 2-aminoethane-1-sulfonamide (351.1 mg, 2.84 mmol, 1.5 eq). After stirring at rt for 12 hr, the reaction mixture was concentrated under reduced pressure. The residue was purified by column on silica gel using petroleum ether and ethyl acetate (1/1, v/v) to give 41.3 (800 mg, 1.16 mmol, 62%) as a white solid. LCMS (ESI+): m/z 618.0 (M+H)⁺.

### Synthesis of 41.4

To a solution of ethyl 41.3 (800 mg, 1.29 mmol, 1 eq) in tetrahydrofuran (40 mL) and H₂O (10 mL) at rt was added LiOH·H₂O (108.64 mg, 2.59 mmol, 2 eq). After stirring at rt for 7 hr, the reaction mixture was concentrated under reduced pressure. The residue was re-dissolved in water (5 mL) and adjusted to pH=2 by aqueous HCl (1 N). The mixture solution was extracted with ethyl acetate (5 mL), and the combined organic phase was concentrated under reduced pressure to afford 41.4 (500 mg), which was used for the next step without further purification. LCMS (ESI+): m/z 589.0 (M+H)⁺.

### Synthesis of Compounds 41 and 42

To a solution of 41.4 (500 mg, 847.36 μmol, 1 eq) in dichloromethane (5 mL) at 0° C. were added 2-methylpropyl carbonochloridate (127.30 mg, 932.09 μmol, 122.41 μL, 1.1 eq) and 4-methylmorpholine (257.12 mg, 2.54 mmol, 279.48 μL, 3 eq). After stirring at rt for 1 hr. NH₃/THF (6M) was added. After stirring at rt for 1 hr, the reaction mixture was concentrated under reduced pressure. The residue was dissolved in ethyl acetate (5 mL), washed with brine (5 mL), dried over sodium sulfate, filtered, and concentrated under reduced pressure. The residue was purified by prep-TLC (ethyl acetate/petroleum ether=1/3) to afford racemate (80 mg, 108.64 μmol, 13%) as a white solid. LCMS (ESI+): m/z 589.9 (M+H)⁺¹H NMR: 400 MHz, DMSO-d₆ δ ppm 3.16-3.22 (m, 2H), 3.54 (d, J=6.68 Hz, 2H), 3.59-3.72 (m, 2H), 3.94-4.11 (m, 1H), 4.41-4.58 (m, 1H), 5.07 (dd, J=11.03, 4.23 Hz, 1H), 7.20-7.28 (m, 3H), 7.29-7.36 (m, 2H), 7.40-7.51 (m, 3H), 7.72 (d, J=8.70 Hz, 2H), 7.85-7.96 (m, 4H), 8.08 (s, 1H), 8.28-8.39 (m, 1H) The racemate (80 mg, 135.80 μmol, 1 eq) was further purified by prep-HPLC and separated by SFC to afford Compound 41 (6.5 mg, 11.03 μmol, 8%) and Compound 42 (6.8 mg, 11.54 μmol, 8.5%) as white solids.

Method of the prep-HPLC: Instrument: Gilson 281 semi-preparative HPLC system; Mobile phase: A: TFA/H₂O=0.075% v/v;B: CAN; Column: C18-1 150*30 mm*5 μm; Flow rate: 25 mL/min; Monitor wavelength: 220 & 254 nm;

| Time | B % |
|---|---|
| 0.0 | 30 |
| 8.0 | 75 |
| 8.1 | 75 |
| 8.2 | 100 |
| 11.2 | 100 |
| 11.3 | 30 |
| 12.5 | 30 |

Method of the SFC: Column: DAICEL CHIRALPAK AD (250 mm*30 mm, 10 μm); Mobile phase A: CO₂; Mobile phase B: [0.1% NH₃H2O ethanol]; B %: 50%, Run time: 12 min.

Compound 41: ¹H NMR (400 MHz, DMSO-d₆) δ ppm 3.25-3.31 (m, 2H), 3.67-3.83 (m, 2H), 4.11 (br dd, J=11.19, 4.19 Hz, 1H), 4.55 (t, J=11.13 Hz, 1H), 5.10 (dd, J=11.19, 4.19 Hz, 1H), 7.03 (s, 2H), 7.20-7.29 (m, 3H), 7.30-7.39 (m, 2H), 7.46 (d, J=8.50 Hz, 2H), 7.53 (br s, 1H), 7.72 (d, J=8.50 Hz, 2H), 7.85-7.92 (m, 2H), 7.93-7.99 (m, 2H), 8.09 (br s, 1H), 8.14-8.23 (m, 1H); LCMS (ESI+): m/z 589.0 (M+H)⁺.

Compound 42: ¹H NMR (400 MHz, DMSO-d₆) δ ppm 3.28 (br s, 2H), 3.74 (br t, J=6.00 Hz, 2H), 4.09 (br dd, J=10.51, 2.88 Hz, 1H), 4.53 (br t, J=11.38 Hz, 1H), 5.09 (br dd, J=10.82, 3.56 Hz, 1H), 5.25-9.53 (m, 1H), 6.91-7.16 (m, 2H), 7.19-7.28 (m, 3H), 7.29-7.37 (m, 2H), 7.41-7.53 (m, 3H), 7.70 (brd, J=8.25 Hz, 2H), 7.85-7.91 (m, 2H), 7.92-7.98 (m, 2H), 8.09 (br s, 1H); LCMS (ESI+): m/z 589.0 (M+H)⁺.

### Synthesis of (S,E)-3-(4-fluorophenyl)-4-phenyl-N-(2-sulfamoylethyl)-N′-((4-(trifluoromethyl)phenyl)sulfonyl)-4,5-dihydro-1H-pyrazole-1-carboximidamide (Compound 43) & (S,E)-3-(4-fluorophenyl)-4-phenyl-N-(2-sulfamoylethyl)-N′-((4-(trifluoromethyl)phenyl)sulfonyl)-4,5-dihydro-1H-pyrazole-1-carboximidamide (Compound 44)

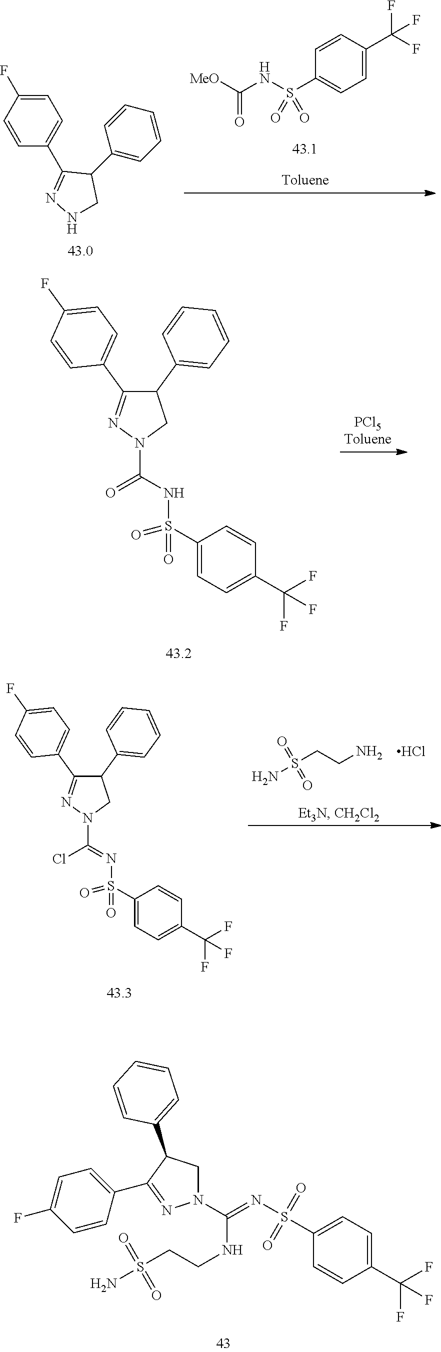

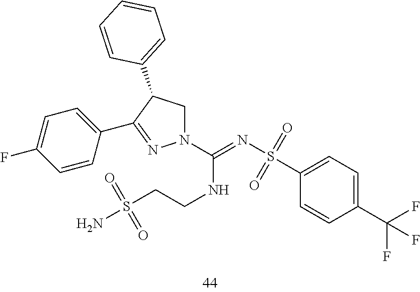 [SMILES: NS(=O)(=O)CCN/C(=N\S(=O)(=O)c1ccc(C(F)(F)F)cc1)N1C[C@@H](c2ccccc2)C(c2ccc(F)cc2)=N1]

### Synthesis of 43.2

A solution of 3-(4-fluorophenyl)-4-phenyl-4,5-dihydro-1H-pyrazole (0.8 g, 3.3 mmol, 43.0, CAS #69433-60-9) and methyl ((4-(trifluoromethyl)phenyl)sulfonyl)carbamate (1.04 g, 3.7 mmol, 43.1, CAS #1042414-31-2) in toluene (30 mL) was stirred at 120 00 for 16 hours. Upon completion, solvent was removed under reduced pressure and the resulting slurry was chromatographed on silica gel eluting with heptane/ethyl acetate mixtures (100/0 to 50/50, v/v) to afford the title product as a brown foam (1.47 g, 2.9 mmol, 91% yield). LC-MS (ESI+): 492.0 (M+H⁺).

### Synthesis of 43.3

To a solution of 3-(4-fluorophenyl)-4-phenyl-N-((4-(trifluoromethyl)phenyl)sulfonyl)-4,5-dihydro-1H-pyrazole-1-carboxamide (1.47 g, 3.0 mmol, 43.2) in toluene (6 mL) at rt was added phosphorus pentachloride (685 mg, 3.29 mmol, 1.1 eq) at 3). After stirring at 120 00 for 4 hours, solvent was removed under reduced pressure. The residue was filtered and purified by flash chromatography on silica gel eluting with heptane/ethyl acetate mixtures (100/0 to 50/50, v/v), affording the expected compound as a white solid (0.7 g, 1.4 mmol, 46% yield). LC-MS (ESI+): 510.00 (M+H⁺).

### Synthesis of Compounds 43 and 44

To a solution of 2-aminoethane-1-sulfonamide hydrochloride (0.09 g, 0.56 mmol, 1.1 eq.) and triethylamine (0.2 mL, 1.43 mmol, 3 eq.) in dichloromethane (5 mL) at rt was added (Z)-3-(4-fluorophenyl)-4-phenyl-N-((4-(trifluoromethyl)phenyl)sulfonyl)-4,5-dihydro-1H-pyrazole-1-carbimidoyl chloride (0.25 g, 0.5 mmol, 43.3). After stirring at rt for 18 hours, the reaction was diluted with dichloromethane (20 mL) and washed with aqueous HCl (2N, 15 mL). The organic layer was dried over magnesium sulfate, filtered, and concentrated in vacuo. The residue was purified by flash chromatography on silica gel eluting with methanol/dichloromethane mixtures (0/100 to 10/90, v/v) to give a colorless oil, which was further subjected to SFC separation conditions, obtaining the following 2 enantiomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%)—iPrOH+0.1% diethylamine, at 35° C., BPR 100 bar. Acquisition frequency was set to 220 nm for the DAD detector.

Compound 43: (33.7 mg, 0.06 mmol, 12% yield, 98% purity, 99% ee). ¹H NMR (DMSO-dB, 400 MHz) δ 8.22 (br s, 1H), 8.0-8.1 (d, J=8.2 Hz, 2H), 7.84 (d, J=8.3 Hz, 2H), 7.7-7.8 (m, 2H), 7.3-7.4 (m, 2H), 7.2-7.3 (m, 5H), 7.03 (s, 2H), 5.10 (dd, J=4.2, 11.1 Hz, 1H), 4.53 (s, 1H), 4.06 (br dd, J=4.2, 11.1 Hz, 1H), 3.73 (br d, J=6.3 Hz, 2H), 3.3-3.3 (m, 2H). LC-MS (ESI+): 598.2 (M+H⁺).

Compound 44: (28.4 mg, 0.05 mmol, 10% yield, 99% purity, 99% ee). ¹H NMR (DMSO-d₆, 400 MHz) δ 8.1-8.3 (s, 1H), 8.03 (d, J=8.2 Hz, 2H), 7.8-7.9 (d, J=8.3 Hz, 2H), 7.7-7.8 (m, 2H), 7.3-7.4 (m, 2H), 7.2-7.3 (m, 5H), 7.03 (s, 2H), 5.1 (m, 1H), 4.53 (s, 1H), 4.0-4.2 (m, 1H), 3.73 (br d, J=6.3 Hz, 2H), 3.3-3.3 (m, 2H). LC-MS (ESI+): 598.3 (M+H⁺).

### Synthesis of (S,E)-N′-((4-chlorophenyl)sulfonyl)-3-(4-fluorophenyl)-4-phenyl-N-(2-sulfamoylethyl)-4,5-dihydro-1H-pyrazole-1-carboximidamide (45) & (R,E)-N′-((4-chlorophenyl)sulfonyl)-3-(4-fluorophenyl)-4-phenyl-N-(2-sulfamoylethyl)-4,5-dihydro-1H-pyrazole-1-carboximidamide (46)

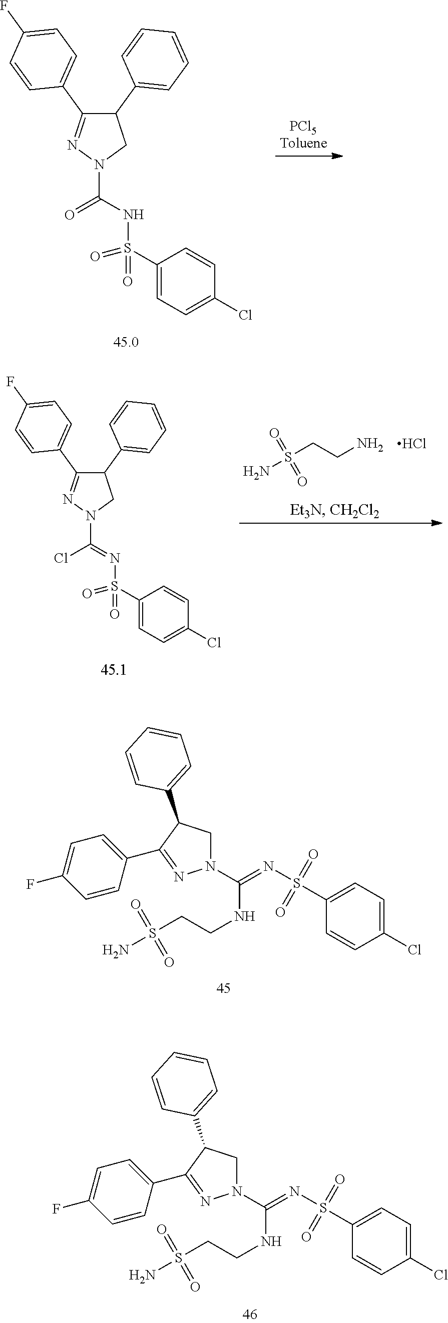 [SMILES: O=C(NS(=O)(=O)c1ccc(Cl)cc1)N1CC(c2ccccc2)C(c2ccc(F)cc2)=N1 ; O=S(=O)(/N=C(\Cl)N1CC(c2ccccc2)C(c2ccc(F)cc2)=N1)c1ccc(Cl)cc1 ; NCCS(N)(=O)=O ; Cl ; NS(=O)(=O)CCN/C(=N\S(=O)(=O)c1ccc(Cl)cc1)N1C[C@H](c2ccccc2)C(c2ccc(F)cc2)=N1 ; NS(=O)(=O)CCN/C(=N\S(=O)(=O)c1ccc(Cl)cc1)N1C[C@@H](c2ccccc2)C(c2ccc(F)cc2)=N1]

### Synthesis of 45.1

To a solution of N-((4-chlorophenyl)sulfonyl)-3-(4-fluorophenyl)-4-phenyl-4,5-dihydro-1H-pyrazole-1-carboxamide (5.86 g, 12.8 mmol, CAS #656827-57-5) in toluene (32 mL) at rt was added phosphorus pentachloride (2.9 g, 13.9 mmol, 1.1 eq). After stirring at 120° C. for 4 hours, solvent was removed under reduced pressure. The residue was washed with diethyl ether (50 mL). The remaining solid was filtered and dried in vacuo to give the expected compound as a pale white solid, which was used in the next step without further purification (1.45 g, 3.05 mmol, 24% yield). LC-MS (ESI+): 476.040 (M+H⁺).

### Synthesis of Compounds 45 and 46

To a solution of 2-aminoethane-1-sulfonamide hydrochloride (0.13 g, 0.81 mmol, 1.5 eq.) and triethylamine (0.37 mL, 2.7 mmol, 5 eq.) in dichloromethane (3 mL) at rt was added (Z)-N-((4-chlorophenyl)sulfonyl)-3-(4-fluorophenyl)-4-phenyl-4,5-dihydro-1H-pyrazole-1-carbimidoyl chloride (0.25 g, 0.53 mmol, 45.1). After stirring at rt for 18 hours, the reaction was diluted with dichloromethane (20 mL) and washed with aqueous HCl (2N, 15 mL). The organic layer was dried over magnesium sulfate, filtered, and concentrated in vacuo. The residue was purified by flash chromatography on silica gel eluting with methanol/dichloromethane mixtures (0/100 to 10/90, v/v) to give a colorless oil, which was further subjected to SFC separation conditions, obtaining the following 2 enantiomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%)—iPrOH+0.1% diethylamine, at 35° C., BPR 100 bar. Acquisition frequency was set to 220 nm for the DAD detector.

Compound 45: (21.4 mg, 0.04 mmol, 7% yield, 99% purity, 99% ee, OR: −70.5). ¹H NMR (DMSO-d₆, 400 MHz) δ 8.1-8.2 (s, 1H), 7.7-7.9 (m, 4H), 7.53 (d, J=8.6 Hz, 2H), 7.3-7.4 (m, 2H), 7.2-7.3 (m, 5H), 7.03 (s, 2H), 5.10 (dd, J=4.4, 11.1 Hz, 1H), 4.52 (t, J=11.2 Hz, 1H), 4.07 (dd, J=4.1, 11.1 Hz, 1H), 3.7-3.8 (m, 2H), S 3.2-3.3 (m, 2H). LC-MS (ESI+): 564.0 (M+H⁺).

Compound 46: (19.8 mg, 0.035 mmol, 7% yield, 99% purity, 99% ee, OR: +19.8). ¹H NMR (DMSO-d₆, 400 MHz) δ 8.16 (br s, 1H), 7.7-7.9 (m, 4H), 7.53 (d, J=8.6 Hz, 2H), 7.3-7.4 (m, 2H), 7.2-7.3 (m, 5H), 7.03 (s, 2H), 5.10 (dd, J=4.3, 11.2 Hz, 1H), 4.52 (s, 1H), 4.07 (dd, J=4.4, 11.1 Hz, 1H), 3.73 (br d, J=5.9 Hz, 2H), S 3.2-3.3 (m, 2H).LC-MS (ESI+): 564.0 (M+H⁺).

### Synthesis of (S,E)-3-(4-fluorophenyl)-4-phenyl-N-((R)-2-sulfamoylpropyl)-N′-((4-(trifluoromethyl)phenyl)sulfonyl)-4,5-dihydro-1H-pyrazole-1-carboximidamide (Compound 47); (S,E)-3-(4-fluorophenyl)-4-phenyl-N-((S)-2-sulfamoylpropyl)-N′-((4-(trifluoromethyl)phenyl)sulfonyl)-4,5-dihydro-1H-pyrazole-1-carboximidamide (48); (R,E)-3-(4-fluorophenyl)-4-phenyl-N-((R)-2-sulfamoylpropyl)-N′-((4-(trifluoromethyl)phenyl)sulfonyl)-4,5-dihydro-1H-pyrazole-1-carboximidamide (47);& (R,E)-3-(4-fluorophenyl)-4-phenyl-N-((S)-2-sulfamoylpropyl)-N′-((4-(trifluoromethyl)phenyl)sulfonyl)-4,5-dihydro-1H-pyrazole-1-carboximidamide (Compound 47)

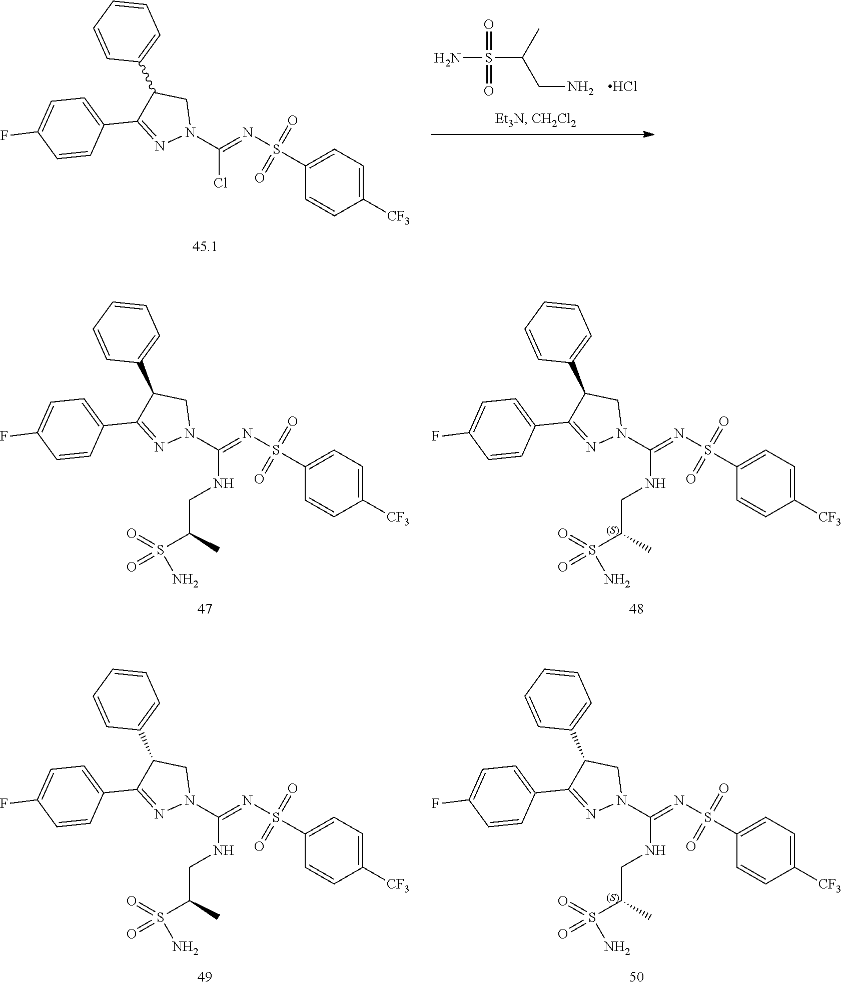

To a stirred solution of 1-aminopropane-2-sulfonamide hydrochloride (0.14 g, 0.81 mmol, 1.5 eq.) and triethylamine (0.23 mL, 1.6 mmol, 3.0 eq.) in dichloromethane (3 mL) at rt was added (E)-3-(4-fluorophenyl)-4-phenyl-N-((4-(trifluoromethyl)phenyl)sulfonyl)-4,5-dihydro-1H-pyrazole-1-carbimidoyl chloride (0.28 g, 0.54 mmol, 45.1). After stirring at rt for 18 hours, the reaction was diluted with dichloromethane (20 mL) and washed with aqueous ammonium chloride solution (15 mL). The organic layer was dried over magnesium sulfate, filtered, and concentrated in vacuo. The residue was purified by flash chromatography on silica eluting with heptane/ethyl acetate mixtures (100/0 to 60/40, v/v) to give a colorless oil as a mixture of diastereomers, which was separated by SFC.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 2.5 ml/min of CO₂ (60%)—MeOH+0.1% diethylamine, at 35° C., BPR 100 bar. Acquisition frequency was set to 220 nm for the DAD detector. Absolute stereochemistry was not determined. Diastereomers with (-) OR were arbitrary assigned as S isomer.

Compound 47: (8.6 mg, 0.014 mmol, 3% yield, 99% purity, 99% ee, OR: −47.6). ¹H NMR (400 MHz, CDCl₃) δ 8.04 (d, J=8.1 Hz, 2H), 7.58-7.70 (m, 5H), 7.27-7.35 (m, 3H), 7.10-7.15 (m, 2H), 6.95-7.02 (m, 2H), 4.88 (br s, 2H), 4.70 (dd, J=11.4, 5.3 Hz, 1H), 4.54 (t, J=11.4 Hz, 1H), 3.92-4.14 (m, 3H), 3.51 (quind, J=7.1, 4.0 Hz, 1H), 1.47 ppm (d, J=7.1 Hz, 3H). LC-MS (ESI+): 612.1 (M+H⁺).

Compound 48: (5.9 mg, 0.010 mmol, 2% yield, 95% purity, 99% ee, OR: −62.5). ¹H NMR (400 MHz, CDCl₃) δ 8.05 (d, J=8.1 Hz, 2H), 7.69 (d, J=8.1 Hz, 2H), 7.62 (br dd, J=8.6, 5.3 Hz, 3H), 7.28-7.36 (m, 3H), 7.13 (br d, J=7.0 Hz, 2H), 6.99 (t, J=8.5 Hz, 2H), 4.82 (br s, 2H), 4.71 (br dd, J=10.9, 4.9 Hz, 1H), 4.52 (br t, J=11.3 Hz, 1H), 4.05-4.15 (m, 2H), 3.93-4.03 (m, 1H), 3.50 (br s, 1H), 1.48 ppm (br d, J=6.5 Hz, 3H). LC-MS (ESI+): 612.1 (M+H⁺).

Compound 49: (11.3 mg, 0.018 mmol, 3% yield, 99% purity, 99% ee, OR: +99.5). ¹H NMR (400 MHz, CDCl₃) δ 8.04 (d, J=8.1 Hz, 2H), 7.67 (d, J=8.2 Hz, 3H), 7.57-7.64 (m, 2H), 7.27-7.35 (m, 3H), 7.09-7.15 (m, 2H), 6.94-7.02 (m, 2H), 4.91 (br s, 2H), 4.70 (dd, J=11.3, 5.2 Hz, 1H), 4.54 (t, J=11.4 Hz, 1H), 3.93-4.14 (m, 3H), 3.51 (td, J=7.2, 3.7 Hz, 1H), 1.47 ppm (d, J=7.0 Hz, 3H). LC-MS (ESI+): 612.1 (M+H⁺).

Compound 50: (10.9 mg, 0.018 mmol, 3% yield, 97% purity, 99% ee, OR: +103.4). ¹H NMR (400 MHz, CDCl₃) δ 8.04 (d, J=8.2 Hz, 2H), 7.58-7.71 (m, 5H), 7.27-7.35 (m, 3H), 7.10-7.16 (m, 2H), 6.94-7.01 (m, 2H), 4.87 (br s, 2H), 4.71 (dd, J=11.3, 5.4 Hz, 1H), 4.52 (t, J=11.4 Hz, 1H), 4.05-4.13 (m, 2H), 3.92-4.02 (m, 1H), 3.50 (dt, J=7.1, 4.0 Hz, 1H), 1.47 ppm (d, J=7.0 Hz, 3H). LC-MS (ESI+): 612.1 (M+H⁺).

### Synthesis of (S)-3-(4-fluorophenyl)-N′-((4-fluorophenyl)sulfonyl)-4-phenyl-N-(2-sulfamoylethyl)-4,5-dihydro-1H-pyrazole-1-carboximidamide (Compound 51) & (R)-3-(4-fluorophenyl)-N′-((4-fluorophenyl)sulfonyl)-4-phenyl-N-(2-sulfamoylethyl)-4,5-dihydro-1H-pyrazole-1-carboximidamide (Compound 52)

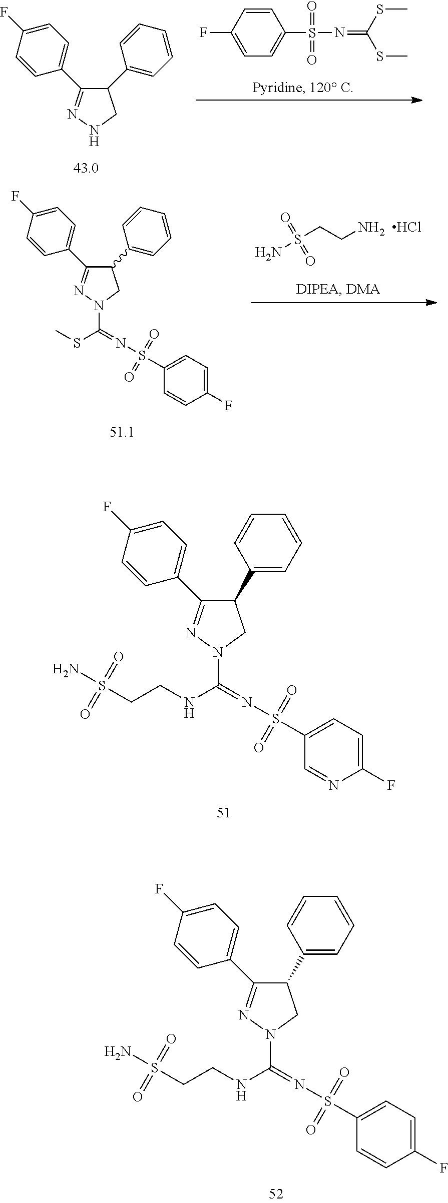

### Synthesis of 51.1

Dimethyl((4-fluorophenyl)sulfonyl)carbonimidodithioate (0.63 g, 2.24 mmol, 1 eq.) and 3-(4-fluorophenyl)-4-phenyl-4,5-dihydro-1H-pyrazole (0.54 g, 2.24 mmol, 43.0) in pyridine (3.3 mL) and was refluxed at 120° C. for 18 h. The reaction mixture was concentrated to dryness and the crude was purified by column chromatography (SiO₂) eluting with heptane/ethyl acetate mixtures (100/0 to 50/50) to afford the desired product as a yellow sticky solid (0.37 g, 0.79 mmol, 35% yield). LC-MS (ESI+): 472.1 (M+H⁺).

### Synthesis of Compounds 51 and 52

2-aminoethane-1-sulfonamide hydrochloride (613 mg, 3.8 mmol, 5 eq.) was added to a stirred solution of methyl 3-(4-fluorophenyl)-N-((4-fluorophenyl)sulfonyl)-4-phenyl-4,5-dihydro-1H-pyrazole-1-carbimidothioate (360 mg, 0.76 mmol, 51.1) and triethylamine (0.64 mL, 4.6 mmol, 6 eq.) in methanol (2.4 mL). After stirring at rt for 18h, solvents were removed under reduced pressure. The residue was chromatographed on silica eluting with dichloromethane/methanol mixtures (100/0 to 90/10) to afford a colorless oil, which was further subjected to SFC purification (Amylose-1, ISOC.50% EtOH+0.1% DEA) to resolve the two enantiomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—EtOH+0.1% diethylamine, at 35° C., BPR 100 bar. Acquisition frequency was set to 220 nm for the DAD detector.

Compound 51: (87.3 mg, 0.16 mmol, 21% yield, 99% purity, 99% ee, OR: −173.7). ¹H NMR (DMSO-d6, 400 MHz) δ 7.8-7.9 (m, 2H), 7.7-7.8 (m, 2H), 7.2-7.4 (m, 10H), 6.9-7.1 (m, 2H), 5.09 (dd, J=4.3, 11.1 Hz, 1H), 4.52 (t, J=11.2 Hz, 1H), 4.08 (dd, J=4.3, 11.1 Hz, 1H), 3.73 (br t, J=6.7 Hz, 2H), 3.2-3.3 (m, 2H). LC-MS (ESI+): 547.6 (M+H⁺).

Compound 52: (77.4 mg, 0.14 mmol, 18% yield, 99% purity, 99% ee, OR: +110.9). ¹H NMR (DMSO-d6, 400 MHz) δ 7.8-7.9 (m, 2H), 7.78 (dd, J=5.5, 8.8 Hz, 2H), 7.0-7.4 (m, 12H), 5.08 (dd, J=4.3, 11.1 Hz, 1H), 4.52 (t, J=11.2 Hz, 1H), 4.07 (dd, J=4.3, 11.2 Hz, 1H), 3.74 (br t, J=6.8 Hz, 2H), 3.2-3.3 (m, 2H). LC-MS (ESI+): 547.6 (M+H⁺).

### Example 53-56: Synthesis of (S,E)-3-(4-fluorophenyl)-N′-((4-fluorophenyl)sulfonyl)-4-phenyl-N-((R)-2-sulfamoylpropyl)-4,5-dihydro-1H-pyrazole-1-carboximidamide (Compound 53); (S,E)-3-(4-fluorophenyl)-N′-((4-fluorophenyl)sulfonyl)-4-phenyl-N-((S)-2-sulfamoylpropyl)-4,5-dihydro-1H-pyrazole-1-carboximidamide (Compound 54); (R,E)-3-(4-fluorophenyl)-N′-((4-fluorophenyl)sulfonyl)-4-phenyl-N-((R)-2-sulfamoylpropyl)-4,5-dihydro-1H-pyrazole-1-carboximidamide (Compound 55) & (R,E)-3-(4-fluorophenyl)-N′-((4-fluorophenyl)sulfonyl)-4-phenyl-N-((S)-2-sulfamoylpropyl)-4,5-dihydro-1H-pyrazole-1-carboximidamide (Compound 56)

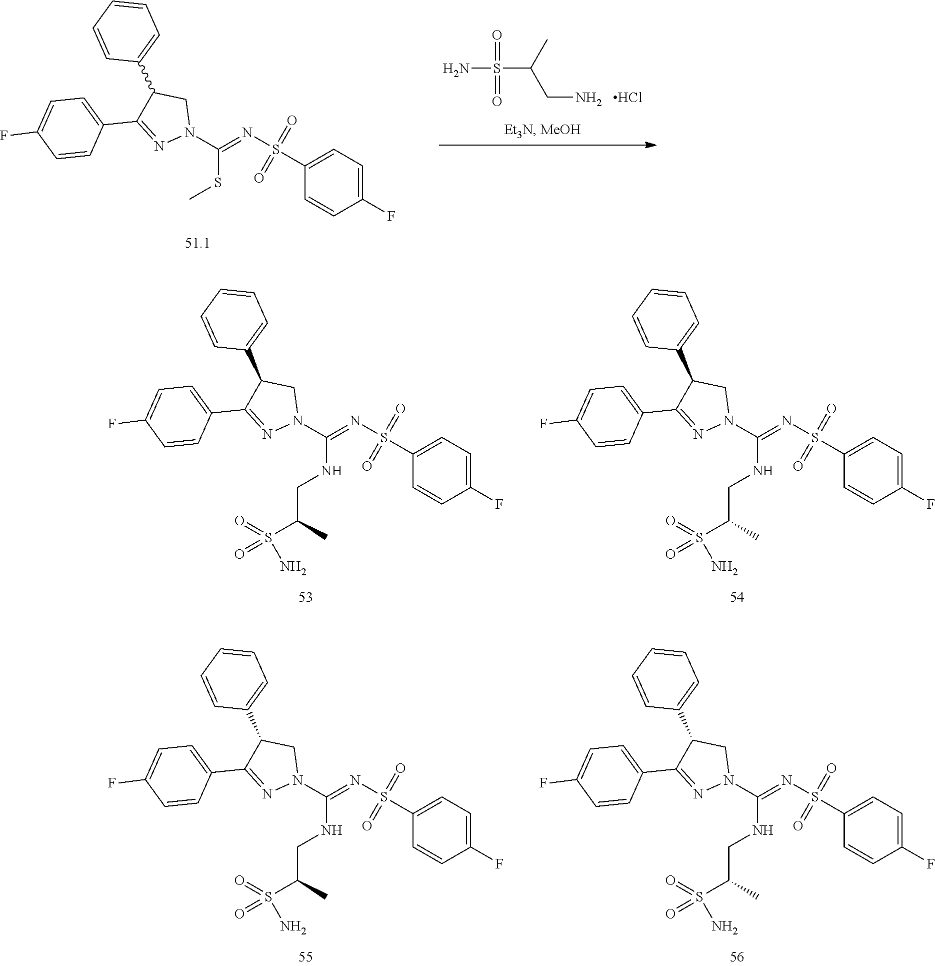

1-aminopropane-2-sulfonamide hydrochloride (194 mg, 1.11 mmol, 2.1 eq.) was added to a stirring solution of methyl 3-(4-fluorophenyl)-N-((4-fluorophenyl)sulfonyl)-4-phenyl-4,5-dihydro-1H-pyrazole-1-carbimidothioate (250 mg, 0.53 mmol, 51.1) and triethylamine (0.23 mL, 1.64 mmol, 3.1 eq.) in methanol (1.20 mL). After stirring at rt for 18 h, the reaction mixture was concentrated to dryness and purified by flash chromatography on silica eluting with dichloromethane/methanol mixtures (100/0 to 90/10) to give a yellowish oil as a mixture of diastereomers which was further subjected to SFC separation.

SFC purification was performed on a Jasco 4000 Parallel SFC System (4× Channels 3×UV+1×DAD) using an Phenomenex column (Lux Cellulose-4) 100 mm long×4.6 mm I.D. 3 μm particle size, on isocratic mode at 2.5 ml/min of 002 (60%)—EtOH-MeOH+0.1% diethylamine, at 35° C., BPR 100 bar. Acquisition frequency was set to 220 nm for the DAD detector.

Compound 53: (25.3 mg, 0.045 mmol, 8% yield, 99% purity, 99% ee, OR: −113.3). ¹H NMR (DMSO-d₆, 400 MHz) δ 8.08 (br s, 1H), 7.8-7.9 (m, 2H), 7.78 (dd, J=5.5, 8.8 Hz, 2H), 7.2-7.4 (m, 9H), 6.97 (br s, 2H), 5.10 (dd, J=4.3, 11.2 Hz, 1H), 4.55 (t, J=11.2 Hz, 1H), 4.0-4.1 (m, 1H), 3.6-3.7 (m, 1H), 3.56 (br d, J=13.3 Hz, 1H), 3.23 (br d, J=6.8 Hz, 1H), 1.16 (d, J=6.8 Hz, 3H). LC-MS (ESI+): 562.1 (M+H⁺).

Compound 54: (36.1 mg, 0.06 mmol, 12% yield, 97% purity, 99% ee, OR: −72.2). ¹H NMR (DMSO-d₆, 400 MHz) δ 8.09 (br t, J=5.6 Hz, 1H), 7.8-7.9 (m, 2H), 7.79 (dd, J=5.5, 8.8 Hz, 1H), 7.2-7.4 (m, 10H), 6.98 (s, 2H), 5.11 (dd, J=4.4, 11.2 Hz, 1H), 4.54 (t, J=11.1 Hz, 1H), 4.14 (dd, J=4.3, 11.2 Hz, 1H), 3.6-3.8 (m, 1H), 3.56 (td, J=6.8, 13.9 Hz, 1H), 3.2-3.3 (m, 1H), 1.16 (d, J=7.0 Hz, 3H). LC-MS (ESI+): 562.1 (M+H⁺).

Compound 55: (23.3 mg, 0.04 mmol, 8% yield, 98% purity, 99% ee, OR: +50.5). ¹H NMR (DMSO-d₆, 400 MHz) δ 8.09 (br t, J=5.4 Hz, 1H), 7.8-7.9 (m, 2H), 7.8 (m, 2H), 7.2-7.4 (m, 9H), 6.97 (s, 2H), 5.11 (dd, J=4.3, 11.1 Hz, 1H), 4.56 (t, J=11.2 Hz, 1H), 4.08 (dd, J=4.3, 11.2 Hz, 1H), 3.6-3.8 (m, 1H), 3.5-3.6 (m, 1H), 3.2-3.3 (m, 1H), 1.17 (d, J=7.0 Hz, 3H). LC-MS (ESI+): 562.1 (M+H⁺).

Compound 56: (23.2 mg, 0.04 mmol, 8% yield, 99% purity, 99% ee, OR: +139.1). ¹H NMR (DMSO-d₆, 400 MHz) δ 8.08 (br d, J=1.4 Hz, 1H), 7.87 (dd, J=5.3, 8.8 Hz, 2H), 7.78 (dd, J=5.5, 8.7 Hz, 2H), 7.2-7.4 (m, 9H), 6.98 (br s, 2H), 5.10 (dd, J=4.2, 11.1 Hz, 1H), 4.54 (t, J=11.1 Hz, 1H), 4.13 (br dd, J=4.3, 11.0 Hz, 1H), 3.67 (br d, J=5.9 Hz, 1H), 3.55 (br dd, J=6.2, 13.6 Hz, 1H), 3.25 (br s, 1H), 1.16 (d, J=6.9 Hz, 3H). LC-MS (ESI+): 562.1 (M+H⁺).

### Synthesis of (S,E)-N′-((4-chlorophenyl)sulfonyl)-3-(4-fluorophenyl)-4-phenyl-N-((R)-1-sulfamoylpropan-2-yl)-4,5-dihydro-1H-pyrazole-1-carboximidamide (Compound 57) & (R,E)-N′-((4-chlorophenyl)sulfonyl)-3-(4-fluorophenyl)-4-phenyl-N-((R)-1-sulfamoylpropan-2-yl)-4,5-dihydro-1H-pyrazole-1-carboximidamide (Compound 58)

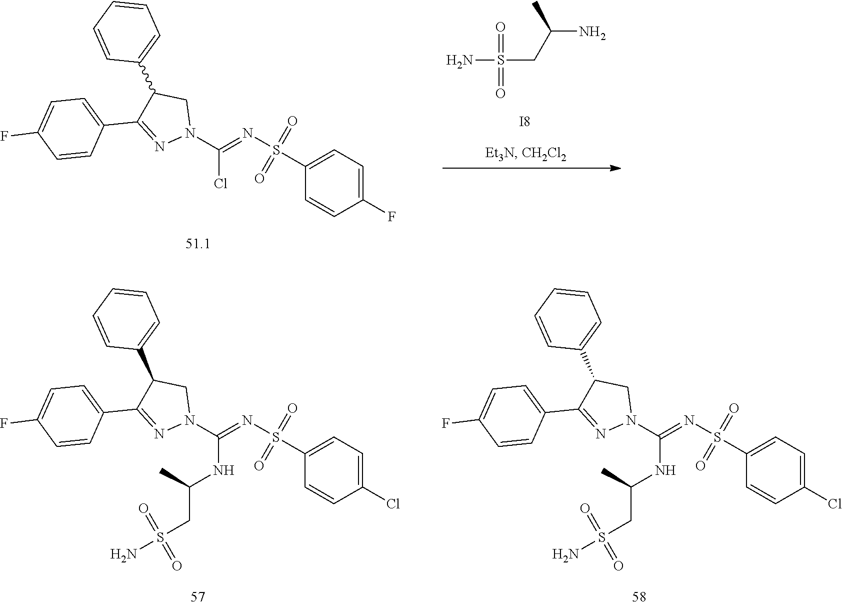

To a solution of (R)-2-aminopropane-1-sulfonamide (131 mg, 0.95 mmol, 1.5 eq., 18) and triethylamine (0.26 mL, 1.89 mmol, 3.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (300 mg, 0.63 mmol, 51.1.). After stirring at rt for 18 hours, the reaction was diluted with dichloromethane (25 mL) and washed with aqueous HCl (2N, 20 mL). The organic layer was dried over magnesium sulfate, filtered, and concentrated in vacuo. The residue was purified by flash chromatography on silica eluting with ethyl acetate/heptane mixtures (0/100 to 70/30, v/v) to give a colorless oil, which was further subjected to SFC separation, giving the following 2 diastereomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 57: (50.9 mg, 0.088 mmol, 14% yield, 99% purity, 99% ee, OR: −112.1). ¹H NMR (DMSO-d₆, 400 MHz) δ 7.94 (br d, J=8.1 Hz, 1H), 7.7-7.9 (m, 4H), 7.54 (d, J=8.6 Hz, 2H), 7.3-7.4 (m, 2H), 7.2-7.3 (m, 5H), 6.96 (s, 2H), 5.09 (dd, J=4.2, 11.1 Hz, 1H), 4.57 (t, J=11.2 Hz, 1H), 4.3-4.5 (m, 1H), 4.08 (br dd, J=3.9, 10.9 Hz, 1H), 3.44 (dd, J=6.2, 14.1 Hz, 1H), 3.2-3.3 (m, 1H), 1.26 (d, J=6.7 Hz, 3H). LC-MS (ESI+): 578.1 (M+H⁺).

Compound 58: (23.2 mg, 0.04 mol, 6% yield, 99% purity, 99% ee, OR: +65.6). ¹H NMR (DMSO-d₆, 400 MHz) δ 7.96 (br d, J=7.8 Hz, 1H), 7.7-7.8 (m, 4H), 7.53 (d, J=8.6 Hz, 2H), 7.3-7.4 (m, 2H), 7.2-7.3 (m, 5H), 6.94 (s, 2H), 5.10 (dd, J=4.3, 11.2 Hz, 1H), 4.52 (t, J=11.1 Hz, 1H), 4.3-4.4 (m, 1H), 4.13 (br dd, J=3.7, 11.0 Hz, 1H), 3.4 (m, 1H), 3.23 (br s, 1H), 1.28 (br d, J=6.7 Hz, 3H). LC-MS (ESI+): 578.0 (M+H⁺).

### Synthesis of (S)-N′-((4-ethynylphenyl)sulfonyl)-3-(4-fluorophenyl)-4-phenyl-N-(2-sulfamoylethyl)-4,5-dihydro-1H-pyrazole-1-carboximidamide (Compound 59) & (R)-N′-((4-ethynylphenyl)sulfonyl)-3-(4-fluorophenyl)-4-phenyl-N-(2-sulfamoylethyl)-4,5-dihydro-1H-pyrazole-1-carboximidamide (Compound 60)

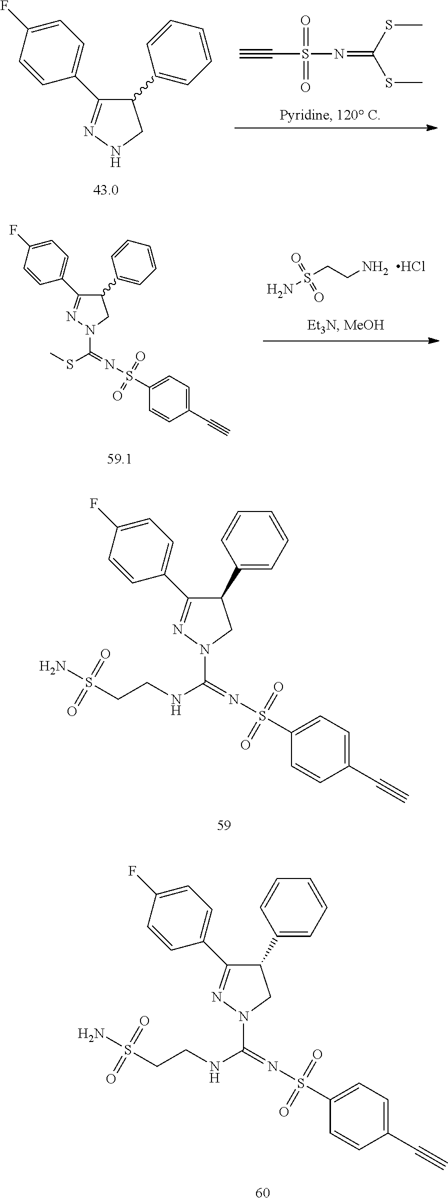

### Synthesis of 59.1

Dimethyl ((4-ethynylphenyl)sulfonyl)carbonimidodithioate (0.39 g, 1.37 mmol, 19) and 3-(4-fluorophenyl)-4-phenyl-4,5-dihydro-1H-pyrazole (0.33 g, 1.37 mmol, 43.0) were mixed in pyridine (2.5 mL). After stirring at 120° C. for 72 h, the reaction mixture was concentrated to dryness. The residue purified by column chromatography eluting with ethyl acetate/heptane mixtures (0/100 to 50/50, v/v) to afford the expected product as a colorless oil (0.092 g, 0.19 mmol, 14% yield). LC-MS (ESI+): 478.1 (M+H⁺).

### Synthesis of Compounds 59 and 60

2-aminoethane-1-sulfonamide hydrochloride (93 mg, 0.58 mmol, 3 eq.) was added to a stirred solution of methyl N-((4-ethynylphenyl)sulfonyl)-3-(4-fluorophenyl)-4-phenyl-4,5-dihydro-1H-pyrazole-1-carbimidothioate (92 mg, 0.19 mmol, 59.1) and triethylamine (0.16 mL, 1.16 mmol, 6 eq.) in methanol (1.9 mL). After stirring at rt for 18 hours, solvents were removed under reduced pressure. The residue was chromatographed on silica eluting with ethyl acetate/heptane mixtures (0/100 to 80/20, v/v) to afford a white solid, which was further subjected to SFC purification to separate the two enantiomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 59: (18.4 mg, 0.033 mmol, 17% yield, 99% purity, 99% ee, OR: −47.3). ¹H NMR (DMSO-d₆, 400 MHz) δ 8.16 (br t, J=5.5 Hz, 1H), 7.7-7.9 (m, 4H), 7.56 (d, J=8.4 Hz, 2H), 7.3-7.4 (m, 2H), 7.2-7.3 (m, 5H), 7.02 (s, 2H), 5.09 (dd, J=4.3, 11.1 Hz, 1H), 4.52 (t, J=11.1 Hz, 1H), 4.39 (s, 1H), 4.08 (dd, J=4.3, 11.1 Hz, 1H), 3.73 (q, J=6.3 Hz, 2H), 3.2-3.3 (m, 2H). LC-MS (ESI+): 554.1 (M+H⁺).

Compound 60: (17.6 mg, 0.032 mmol, 17% yield, 99% purity, 99% ee, OR: +146.9). ¹H NMR (DMSO-d₆, 400 MHz) δ 8.16 (br t, J=5.5 Hz, 1H), 7.7-7.8 (m, 4H), 7.56 (d, J=8.4 Hz, 2H), 7.3-7.4 (m, 2H), 7.2-7.3 (m, 5H), 7.02 (s, 2H), 5.09 (dd, J=4.3, 11.1 Hz, 1H), 4.52 (t, J=11.2 Hz, 1H), 4.39 (s, 1H), 4.08 (dd, J=4.3, 11.0 Hz, 1H), 3.73 (q, J=6.3 Hz, 2H), 3.2-3.3 (m, 2H). LC-MS (ESI+): 554.1 (M+H⁺).

### Synthesis of (S,E)-N′-((4-chlorophenyl)sulfonyl)-3-(4-fluorophenyl)-4-phenyl-N-(1-sulfamoylpropan-2-yl)-4,5-dihydro-1H-pyrazole-1-carboximidamide (Compound 61) & (R,E)-N′-((4-chlorophenyl)sulfonyl)-3-(4-fluorophenyl)-4-phenyl-N-(1-sulfamoylpropan-2-yl)-4,5-dihydro-1H-pyrazole-1-carboximidamide (Compound 62)

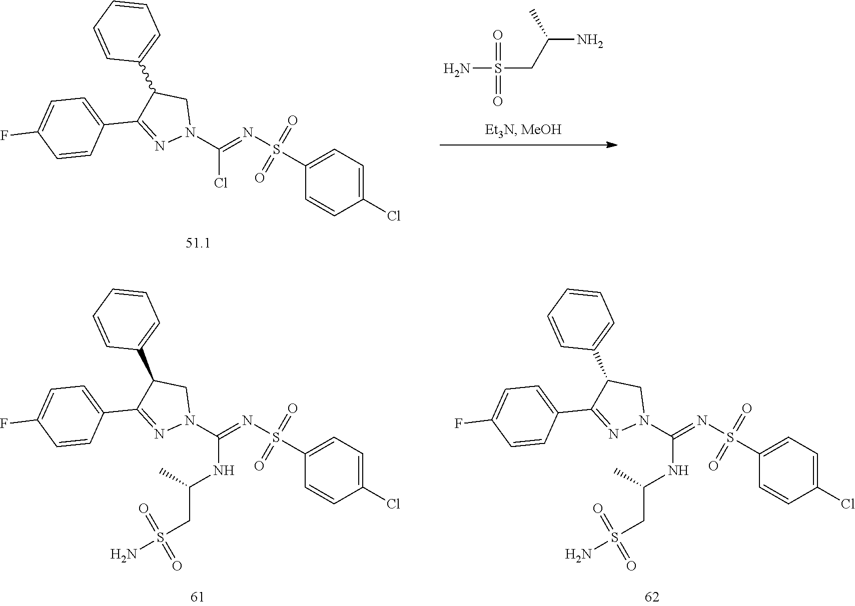

To a solution of (S)-2-aminopropane-1-sulfonamide (87 mg, 0.63 mmol, 1.5 eq.) and triethylamine (0.18 mL, 1.26 mmol, 3.0 eq.) in dichloromethane/methanol (3:1) (4 mL) was added (Z)-N-((4-chlorophenyl)sulfonyl)-3-(4-fluorophenyl)-4-phenyl-4,5-dihydro-1H-pyrazole-1-carbimidoyl chloride (200 mg, 0.42 mmol, 51.1). After stirring at rt for 18 hours, solvents were removed under reduced pressure. The residue was purified by flash chromatography on silica eluting with ethyl acetate/heptane mixtures (0/100 to 80/20, v/v) to afford a white solid which was further subjected to SFC purification to separate the two enantiomers.

SFC purification was performed on a Jasco 4000 Parallel SFC System (4× Channels 3×UV+1×DAD) using an Phenomenex column (Lux Cellulose-1) 150 mm long×4.6 mm I.D. 5 μm particle size, on isocratic mode at 3 ml/min of CO₂ (40%)—MeOH+0.1% diethylamine, at 35° C., BPR 100 bar. Acquisition frequency was set to 220 nm for the DAD detector.

Compound 61: (20.8 mg, 0.036 mol, 9% yield, 96% purity, 99% de, OR: −84.4). ¹H NMR (DMSO-d₆, 400 MHz) δ 7.8 (m, 4H), 7.6-7.7 (m, 2H), 7.3-7.4 (m, 3H), 7.23 (t, J=8.6 Hz, 6H), 5.02 (dd, J=4.2, 11.2 Hz, 1H), 4.27 (t, J=11.3 Hz, 1H), 3.9-4.0 (m, 1H), 3.77 (dd, J=4.2, 11.3 Hz, 1H), 3.3-3.4 (m, 2H), 2.88 (dd, J=11.0, 13.3 Hz, 1H), 1.42 (d, J=6.5 Hz, 3H). LC-MS (ESI+): 578.0 (M+H⁺).

Compound 62: (32.1 mg, 0.056 mmol, 13% yield, 98% purity, 99% de, OR: +198.1). ¹H NMR (DMSO-d₆, 400 MHz) δ 7.94 (br d, J=8.3 Hz, 1H), 7.7-7.9 (m, 4H), 7.54 (d, J=8.5 Hz, 2H), 7.3-7.4 (m, 2H), 7.2-7.3 (m, 5H), 6.96 (s, 2H), 5.09 (dd, J=4.1, 11.1 Hz, 1H), 4.57 (t, J=11.2 Hz, 1H), 4.3-4.5 (m, 1H), 4.08 (br dd, J=4.0, 11.2 Hz, 1H), 3.44 (dd, J=6.3, 14.1 Hz, 1H), 3.25 (dd, J=6.1, 14.1 Hz, 1H), 1.26 (d, J=6.7 Hz, 3H). LC-MS (ESI+): 578.1 (M+H⁺).

### Synthesis of (S,E)-N′-((4-chlorophenyl)sulfonyl)-3-(4-methoxyphenyl)-N-((S)-3-methyl-1-sulfamoylbutan-2-yl)-4-phenyl-4,5-dihydro-1H-pyrazole-1-carboximidamide (Compound 63) & (R,E)-N′-((4-chlorophenyl)sulfonyl)-3-(4-methoxyphenyl)-N-((S)-3-methyl-1-sulfamoylbutan-2-yl)-4-phenyl-4,5-dihydro-1H-pyrazole-1-carboximidamide (Compound 64)

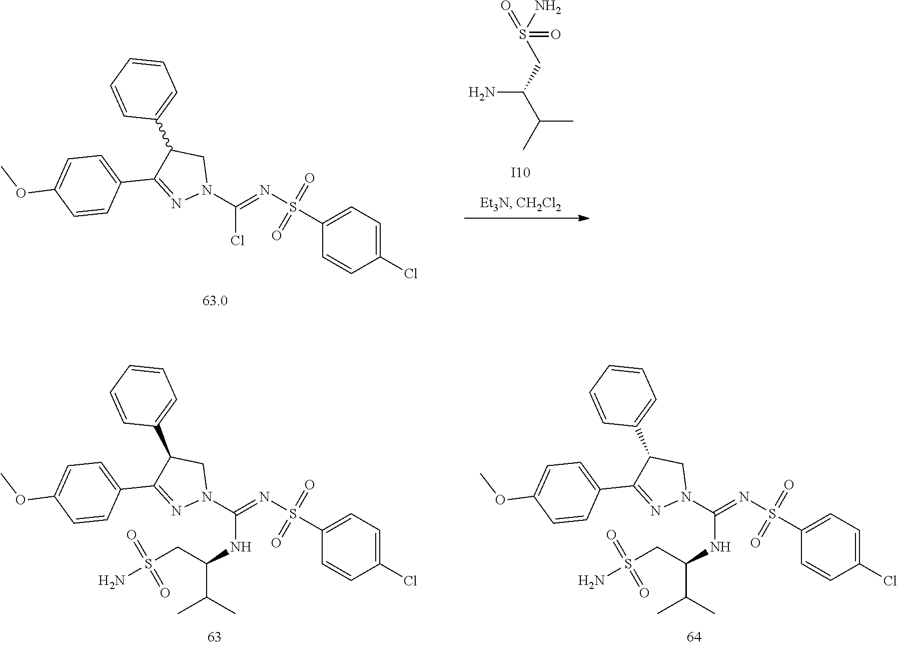

To a solution of (S)-2-amino-3-methylbutane-1-sulfonamide (94 mg, 0.57 mmol, 1.1 eq., I10) and triethylamine (0.21 mL, 1.49 mmol, 3.0 eq.) in dichloromethane (5 mL) at rt was added (Z)-3-(4-methoxyphenyl)-4-phenyl-N-((4-(chlorophenyl)sulfonyl)-4,5-dihydro-1H-pyrazole-1-carbimidoyl chloride (250 mg, 0.51 mmol, 63.0, CAS #1309448-30-3). After stirring at rt for 18 hours, the reaction was diluted with dichloromethane (25 mL) and washed with aqueous HCl (2N, 20 mL). The organic layer was dried over magnesium sulfate, filtered, and concentrated in vacuo. The residue was purified by flash chromatography on silica eluting with methanol/dichloromethane mixtures (0/100 to 10/90, v/v) to give a colorless oil, which was further subjected to SFC separation conditions, obtaining the following 2 diastereomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%)—iPrOH+0.1% diethylamine, at 35° C., BPR 100 bar. Acquisition frequency was set to 220 nm for the DAD detector.

Compound 63: (7.1 mg, 0.011 mmol, 3% yield, 99% purity, 99% ee, OR: −91.8). ¹H NMR (DMSO-d₆, 400 MHz) δ 7.81 (d, J=8.7 Hz, 2H), 7.66 (d, J=8.9 Hz, 2H), 7.55 (d, J=8.7 Hz, 2H), 7.32 (d, J=7.3 Hz, 2H), 7.2-7.3 (m, 3H), 6.92 (d, J=9.0 Hz, 2H), 6.86 (br s, 2H), 5.09 (br d, J=3.9 Hz, 1H), 4.47 (br t, J=11.0 Hz, 1H), 4.2-4.4 (m, 1H), 3.9-4.1 (m, 1H), 3.74 (s, 3H), 3.4-3.5 (m, 2H), 3.2-3.2 (m, 1H), 1.90 (br d, J=1.0 Hz, 1H), 0.7-0.8 (m, 6H). LC-MS (ESI+): 618.1 (M+H⁺).

Compound 64: (22.3 mg, 0.04 mol, 7% yield, 99% purity, 99% ee, OR: +105.5). ¹H NMR (DMSO-d₆, 400 MHz) δ 7.8-7.9 (m, 2H), 7.70 (d, J=8.9 Hz, 2H), 7.5-7.6 (m, 2H), 7.31 (d, J=7.1 Hz, 2H), 7.2-7.3 (m, 3H), 6.93 (d, J=8.9 Hz, 2H), 6.88 (br s, 2H), 5.04 (br d, J=9.9 Hz, 1H), 4.6-4.8 (m, 1H), 3.9-4.2 (m, 2H), 3.75 (s, 3H), 3.45 (br dd, J=9.0, 13.6 Hz, 1H), 3.1-3.3 (m, 1H), 1.7-2.0 (m, 1H), 0.76 (br d, J=6.7 Hz, 6H). LC-MS (ESI+): 618.172 (M+H⁺).

### Synthesis of (S)-3-(4-chlorophenyl)-4-phenyl-N′-(pyridin-3-ylsulfonyl)-N-(2-sulfamoylethyl)-4,5-dihydro-1H-pyrazole-1-carboximidamide (Compound 65) and (R)-3-(4-chlorophenyl)-4-phenyl-N′-(pyridin-3-ylsulfonyl)-N-(2-sulfamoylethyl)-4,5-dihydro-1H-pyrazole-1-carboximidamide (Compound 66)

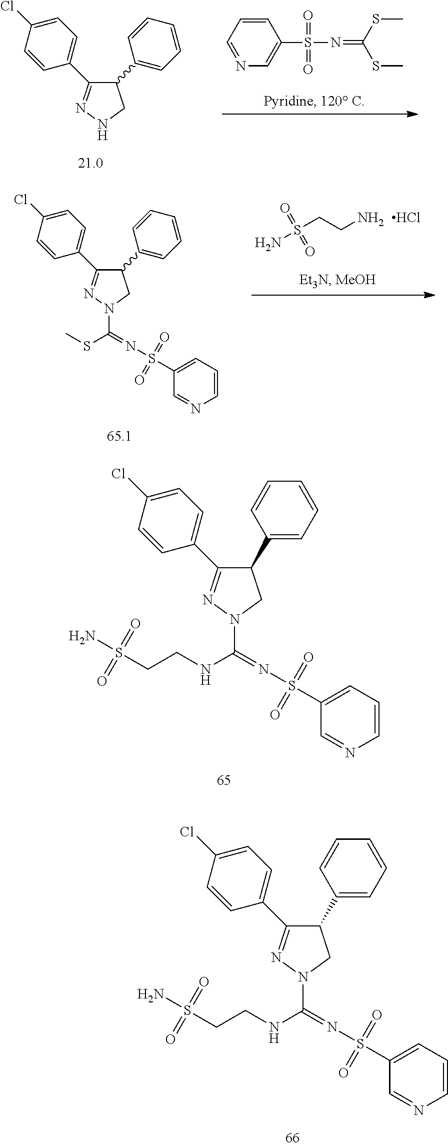

### Synthesis of 65.1

Dimethyl (pyridin-3-ylsulfonyl)carbonimidodithioate (0.12 g, 0.47 mmol, CAS #76511-39-2) and 3-(4-chlorophenyl)-4-phenyl-4,5-dihydro-1H-pyrazole (0.12 g, 0.47 mmol, 21.0) were mixed in pyridine (1 mL). After refluxing for 18 h, the reaction mixture was concentrated to dryness. The residue was purified by column chromatography eluting with ethyl acetate/heptane mixtures (0/100 to 80/20, v/v) to afford the expected product as a colorless oil (0.11 g, 0.23 mmol, 50% yield). LC-MS (ESI+): 471.0 (M+H⁺).

### Synthesis of Compounds 65 and 66

2-aminoethane-1-sulfonamide hydrochloride (76 mg, 0.5 mmol, 2 eq.) was added to a stirred solution of methyl 3-(4-chlorophenyl)-4-phenyl-N-(pyridin-3-ylsulfonyl)-4,5-dihydro-1H-pyrazole-1-carbimidothioate (112 mg, 0.24 mmol, 65.1) and triethylamine (0.1 mL, 0.7 mmol, 3 eq.) in methanol (2.3 mL). After stirring at rt for 18 hours, solvents were removed under reduced pressure. The residue was chromatographed on silica eluting with ethyl acetate/heptane mixtures (0/100 to 50/50) to give a colorless oil, which was further subjected to SFC purification (Amylose-1, ISOC.60% MeOH+0.1% DEA) to afford the two enantiomers.

SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO2 (40%)—MeOH+0.1% diethylamine, at 35° C., BPR 100 bar. Acquisition frequency was set to 220 nm for the DAD detector.

Compound 65: (33.0 mg, 0.06 mmol, 25% yield, 95% purity, 99% ee, OR: −68.2). ¹H NMR (DMSO-d₆, 400 MHz) δ 8.98 (d, J=2.2 Hz, 1H), 8.71 (dd, J=1.2, 4.8 Hz, 1H), 8.2 (m, 2H), 7.72 (d, J=8.6 Hz, 2H), 7.52 (dd, J=4.9, 8.0 Hz, 1H), 7.45 (d, J=8.5 Hz, 2H), 7.3-7.4 (m, 2H), 7.2-7.3 (m, 3H), 7.00 (s, 2H), 5.11 (dd, J=4.4, 11.2 Hz, 1H), 4.56 (t, J=11.2 Hz, 1H), 4.10 (dd, J=4.4, 11.1 Hz, 1H), 3.7-3.8 (m, 2H), 3.2-3.3 (m, 2H). LC-MS (ESI+): 547.0 (M+H⁺).

Compound 66: (16.6 mg, 0.03 mmol, 13% yield, 99% purity, 99% ee, OR: +102.5). ¹H NMR (DMSO-d₆, 400 MHz) δ 8.98 (d, J=2.1 Hz, 1H), 8.71 (dd, J=1.4, 4.8 Hz, 1H), 8.20 (td, J=1.8, 8.1 Hz, 2H), 7.72 (d, J=8.7 Hz, 2H), 7.53 (dd, J=4.8, 7.9 Hz, 1H), 7.45 (d, J=8.7 Hz, 2H), 7.3-7.4 (m, 2H), 7.2-7.3 (m, 3H), 7.00 (s, 2H), 5.11 (dd, J=4.4, 11.2 Hz, 1H), 4.56 (t, J=11.2 Hz, 1H), 4.10 (dd, J=4.4, 11.1 Hz, 1H), 3.74 (q, J=6.3 Hz, 2H), 3.2-3.3 (m, 2H). LC-MS (ESI+): 547.0 (M+H⁺).

### Synthesis of (S)-3-(4-chlorophenyl)-N′-((1-methyl-1H-imidazol-4-yl)sulfonyl)-4-phenyl-N-(2-sulfamoylethyl)-4,5-dihydro-1H-pyrazole-1-carboximidamide (Compound 67) & (R)-3-(4-chlorophenyl)-N′-((1-methyl-1H-imidazol-4-yl)sulfonyl)-4-phenyl-N-(2-sulfamoylethyl)-4,5-dihydro-1H-pyrazole-1-carboximidamide (Compound 68)

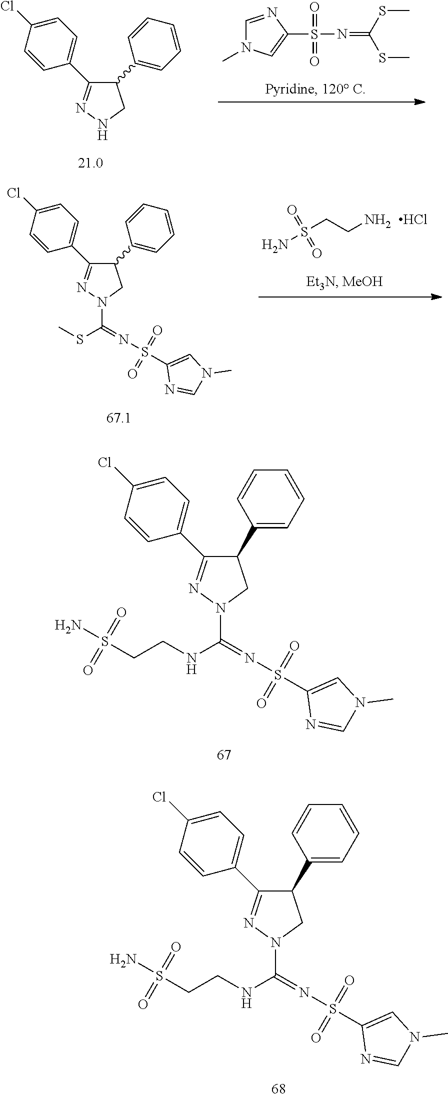

### Synthesis of 67.1

Dimethyl ((1-methyl-1H-imidazol-4-yl)sulfonyl)carbonimidodithioate (65 mg, 0.24 mmol, 111) and 3-(4-chlorophenyl)-4-phenyl-4,5-dihydro-1H-pyrazole (63 mg, 0.24 mmol, 21.0) were mixed in pyridine (1.5 mL). After stirring at reflux for 18 h, the reaction mixture was concentrated to dryness affording the expected product as a yellowish oil (110 mg, 0.23 mmol, 95% yield). LC-MS (ESI+): 474.1 (M+H⁺).

### Synthesis of Compound 67 and 68

2-Aminoethane-1-sulfonamide hydrochloride (75 mg, 0.5 mmol, 2 eq.) was added to a stirring solution of methyl 3-(4-chlorophenyl)-N-((1-methyl-1H-imidazol-4-yl)sulfonyl)-4-phenyl-4,5-dihydro-1H-pyrazole-1-carbimidothioate (110 mg, 0.23 mmol, 67.1) and triethylamine (0.1 mL, 0.7 mmol, 3 eq.) in methanol (1.5 mL) at room temperature. After stirring for 18 hours, solvents were removed under reduced pressure. The residue was chromatographed on silica eluting with ethyl acetate/heptane mixtures (0/100 to 60/40). The product obtained was repurified by flash chromatography on silica eluting with methanol/dichloromethane mixtures (0/100 to 10/90, v/v). The colorless oil obtained was further subjected to SFC purification to afford the two enantiomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—MeOH+0.1% diethylamine, at 35° C., BPR 100 bar. Acquisition frequency was set to 220 nm for the DAD detector.

Compound 67: (8.4 mg, 0.015 mmol, 7% yield, 99% purity, 99% ee, OR: −43.9). ¹H NMR (CDCl₃, 400 MHz) δ 7.8-8.1 (m, 2H), 7.53 (br d, J=8.4 Hz, 2H), 7.44 (s, 1H), 7.33 (m, 1H), 7.2-7.3 (m, 4H), 7.11 (br d, J=7.2 Hz, 2H), 6.35 (br s, 2H), 4.6-4.7 (m, 2H), 4.14 (br s, 3H), 3.7-3.8 (m, 3H), 3.55 (br s, 2H). LC-MS (ESI+): 550.0 (M+H⁺).

Compound 68: (5.6 mg, 0.01 mmol, 5% yield, 97% purity, 99% ee, OR: +27.4). ¹H NMR (CDCl₃, 400 MHz) δ 7.9-8.3 (m, 2H), 7.4-7.6 (m, 3H), 7.1-7.3 (m, 7H), 6.3-6.6 (m, 2H), 4.6-4.8 (m, 2H), 4.0-4.2 (m, 3H), 3.74 (br s, 3H), 3.5-3.6 (m, 2H). LC-MS (ESI+): 550.0 (M+H⁺).

### Synthesis of (S)-3-(4-fluorophenyl)-4-phenyl-N′-(pyridin-3-ylsulfonyl)-N-(2-sulfamoylethyl)-4,5-dihydro-1H-pyrazole-1-carboximidamide (Compound 69) & (R)-3-(4-fluorophenyl)-4-phenyl-N′-(pyridin-3-ylsulfonyl)-N-(2-sulfamoylethyl)-4,5-dihydro-1H-pyrazole-1-carboximidamide (Compound 70)

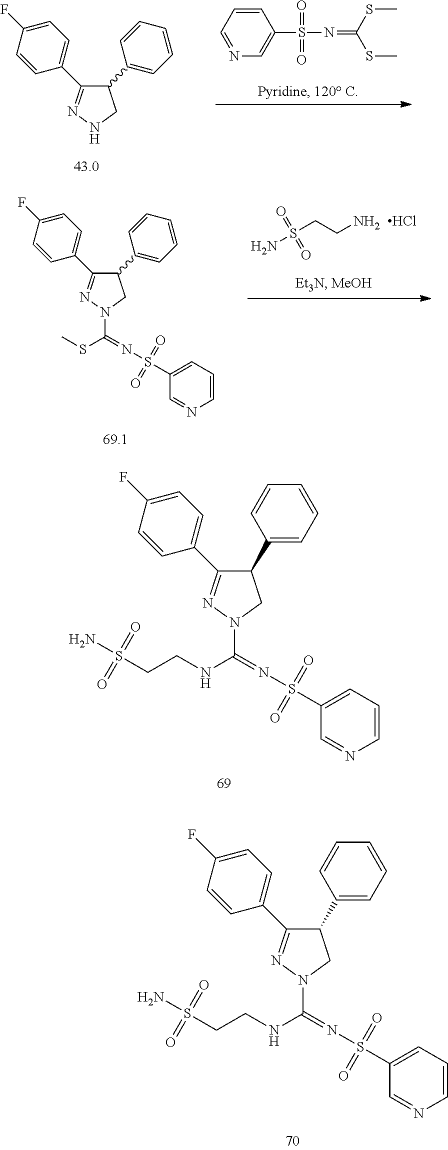

### Synthesis of 69.1

Dimethyl (pyridin-3-ylsulfonyl)carbonimidodithioate (0.27 g, 1.04 mmol, 1 eq.) and 3-(4-fluorophenyl)-4-phenyl-4,5-dihydro-1H-pyrazole (0.25 g, 1.04 mmol, 43.0) in pyridine (5 mL) were refluxed at 120° C. for 18 h. Upon completion, the reaction mixture was concentrated to dryness and the yellowish crude oil was used in the next step without further purification (0.47 g, 1.03 mmol, 99% yield). LC-MS (ESI+): 455.1 (M+H⁺).

### Synthesis of Compounds 69 and 70

2-aminoethane-1-sulfonamide hydrochloride (332 mg, 2.07 mmol, 2 eq.) was added to a stirring solution of methyl 3-(4-fluorophenyl)-4-phenyl-N-(pyridin-3-ylsulfonyl)-4,5-dihydro-1H-pyrazole-1-carbimidothioate (470 mg, 1.03 mmol, 69.1) and triethylamine (0.43 mL, 3.1 mmol, 3 eq.) in methanol (6 mL). After stirring at room temperature for 18 hours, solvents were removed under reduced pressure. The residue was chromatographed on silica eluting with methanol/dichloromethane mixtures (0/100 to 10/90, v/v) to obtain a colorless oil, which was further subjected to SFC purification to resolve the two enantiomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—MeOH+0.1% diethylamine, at 35° C., BPR 100 bar. Acquisition frequency was set to 220 nm for the DAD detector.

Compound 69: (26.7 mg, 0.05 mmol, 5% yield, 98% purity, 99% ee, OR: −108.9). ¹H NMR (DMSO-d₆, 400 MHz) δ 8.98 (d, J=2.2 Hz, 1H), 8.71 (dd, J=1.4, 4.8 Hz, 1H), 8.20 (td, J=1.8, 8.0 Hz, 2H), 7.77 (dd, J=5.6, 8.7 Hz, 2H), 7.52 (dd, J=4.9, 8.0 Hz, 1H), 7.3-7.4 (m, 2H), 7.2-7.3 (m, 5H), 7.02 (br s, 2H), 5.11 (dd, J=4.2, 11.1 Hz, 1H), 4.55 (t, J=11.1 Hz, 1H), 4.10 (dd, J=4.3, 11.1 Hz, 1H), 3.7-3.8 (m, 2H), 3.2-3.3 (m, 2H). LC-MS (ESI+): 531.1 (M+H⁺).

Compound 70: (75.2 mg, 0.14 mmol, 14% yield, 99% purity, 99% ee, OR: +66.7). ¹H NMR (DMSO-d6, 400 MHz) δ 8.98 (d, J=2.1 Hz, 1H), 8.71 (dd, J=1.3, 4.8 Hz, 1H), 8.20 (td, J=1.8, 8.0 Hz, 1H), 7.77 (dd, J=5.6, 8.6 Hz, 2H), 7.52 (dd, J=4.9, 8.0 Hz, 1H), 7.3-7.4 (m, 2H), 7.2-7.3 (m, 6H), 6.9-7.2 (m, 2H), 5.11 (dd, J=4.3, 11.2 Hz, 1H), 4.54 (t, J=11.2 Hz, 1H), 4.10 (dd, J=4.3, 11.1 Hz, 1H), 3.74 (br t, J=6.7 Hz, 2H), 3.2-3.3 (m, 2H). LC-MS (ESI+): 531.1 (M+H⁺).

### Synthesis of (S)-3-(4-chlorophenyl)-N′-((2-isopropyl-2H-1,2,3-triazol-4-yl)sulfonyl)-4-phenyl-N-(2-sulfamoylethyl)-4,5-dihydro-1H-pyrazole-1-carboximidamide (Compound 71) & (R)-3-(4-chlorophenyl)-N′-((2-isopropyl-2H-1,2,3-triazol-4-yl)sulfonyl)-4-phenyl-N-(2-sulfamoylethyl)-4,5-dihydro-1H-pyrazole-1-carboximidamide (Compound 72)

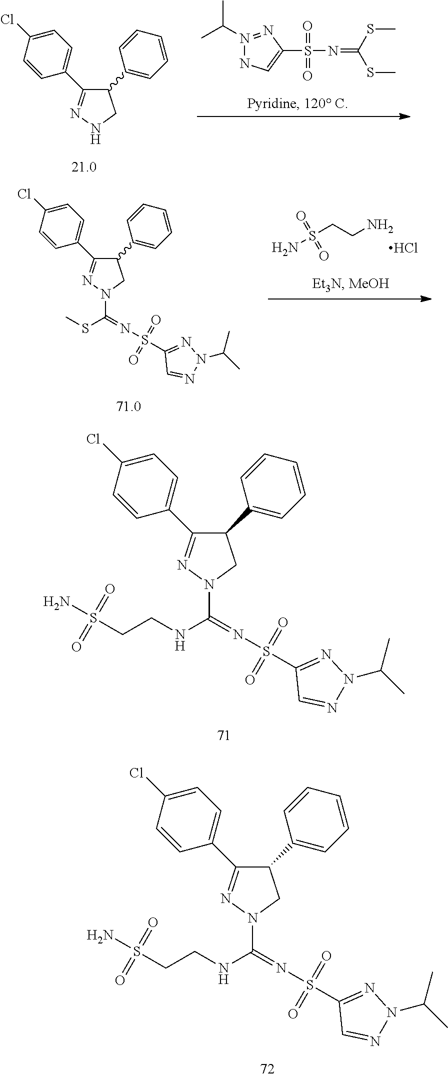

### Synthesis of 71.1

Dimethyl ((2-isopropyl-2H-1,2,3-triazol-4-yl)sulfonyl)carbonimidodithioate (1.45 g, 4.9 mmol, 112) and 3-(4-chlorophenyl)-4-phenyl-4,5-dihydro-1H-pyrazole (1.3 g, 4.9 mmol, 21.0) in pyridine (7 mL) were refluxed at 120 0n for 18 h. Upon completion, the reaction mixture was concentrated to dryness and the residue was purified by flash chromatography on silica eluting with ethyl acetate/heptane mixtures (0/100 to 50/50, v/v) to afford the expected product as a yellow oil (1.09 g, 2.2 mmol, 44% yield). LC-MS (ESI+): 503.1 (M+H⁺).

### Synthesis of Compounds 71 and 72

2-Aminoethane-1-sulfonamide hydrochloride (0.2 g, 1.24 mmol, 2.5 eq.) was added to a stirring solution of methyl (Z)-3-(4-chlorophenyl)-N-((2-isopropyl-2H-1,2,3-triazol-4-yl)sulfonyl)-4-phenyl-4,5-dihydro-1H-pyrazole-1-carbimidothioate (0.25 g, 0.5 mmol, 71.1) and triethylamine (0.24 mL, 1.74 mmol, 3.5 eq.) in methanol (2 mL). After stirring at room temperature for 18 hours, solvents were removed under reduced pressure. The residue was chromatographed on silica eluting with methanol/dichloromethane mixtures (0/100 to 10/90) to obtain a colorless oil, which was further subjected to SFC purification to resolve the two enantiomers.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—MeOH+0.1% diethylamine, at 35° C., BPR 100 bar. Acquisition frequency was set to 220 nm for the DAD detector.

Compound 71: (30.1 mg, 0.052 mmol, 10% yield, 95% purity, 99% ee, OR: −92.3). ¹H NMR (DMSO-d₆, 400 MHz) δ 8.01 (s, 1H), 7.74 (d, J=8.6 Hz, 2H), 7.46 (d, J=8.6 Hz, 2H), 7.31-7.36 (m, 2H), 7.21-7.28 (m, 3H), 5.10 (dd, J=11.2, 4.2 Hz, 1H), 4.79 (spt, J=6.6 Hz, 1H), 4.53 (t, J=11.2 Hz, 1H), 4.09 (br dd, J=11.1, 4.1 Hz, 1H), 3.77 (br t, J=6.8 Hz, 2H), 3.27 (m, 5H), 1.43 (dd, J=6.7, 2.1 Hz, 6H) ppm. LC-MS (ESI+): 579.1 (M+H⁺).

Compound 72: (28.8 mg, 0.05 mmol, 10% yield, 98% purity, 99% ee, OR: +97.1). ¹H NMR (DMSO-d₆, 400 MHz) δ 8.01 (s, 1H), 7.73 (d, J=8.6 Hz, 2H), 7.45 (d, J=8.6 Hz, 2H), 7.29-7.35 (m, 2H), 7.20-7.29 (m, 3H), 5.08 (dd, J=11.2, 4.2 Hz, 1H), 4.79 (spt, J=6.6 Hz, 1H), 4.53 (t, J=11.3 Hz, 1H), 4.08 (br dd, J=11.2, 4.1 Hz, 1H), 3.77 (br t, J=6.8 Hz, 2H), 3.26-3.29 (m, 5H), 1.43 (dd, J=6.7, 2.0 Hz, 6H) ppm. LC-MS (ESI+): 579.1 (M+H⁺).

### Synthesis of (S)-3-(4-chlorophenyl)-N′-((2-methyl-2H-1,2,3-triazol-4-yl)sulfonyl)-4-phenyl-N-(2-sulfamoylethyl)-4,5-dihydro-1H-pyrazole-1-carboximidamide (Compound 73) & (R)-3-(4-chlorophenyl)-N′-((2-methyl-2H-1,2,3-triazol-4-yl)sulfonyl)-4-phenyl-N-(2-sulfamoylethyl)-4,5-dihydro-1H-pyrazole-1-carboximidamide (Compound 74)

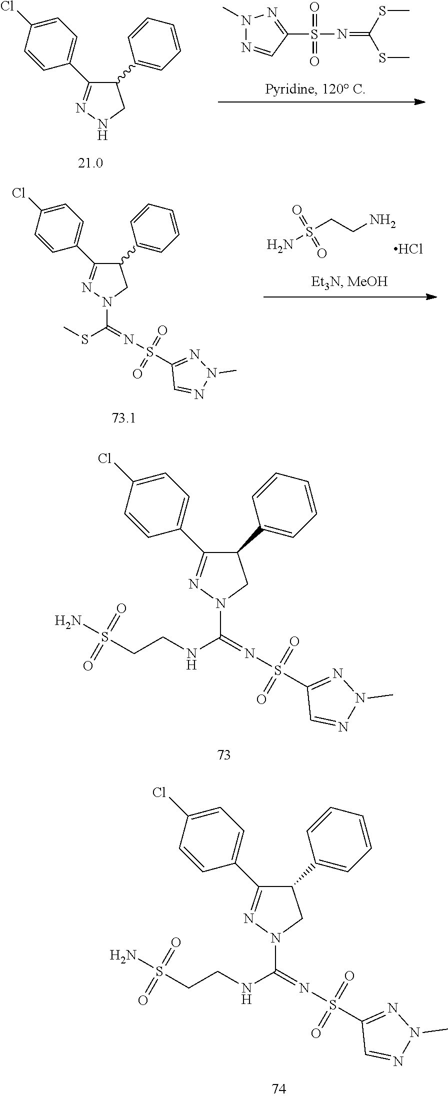

### Synthesis of Methyl 3-(4-chlorophenyl)-N-((2-methyl-2H-1,2,3-triazol-4-yl)sulfonyl)-4-phenyl-4,5-dihydro-1H-pyrazole-1-carbimidothioate (73.1)

Dimethyl ((2-methyl-2H-1,2,3-triazol-4-yl)sulfonyl)carbonimidodithioate (1.17 g, 4.4 mmol, 113) and 3-(4-chlorophenyl)-4-phenyl-4,5-dihydro-1H-pyrazole (1.1 g, 4.4 mmol, 21.0) in pyridine (6 mL) was refluxed at 120 0C for 18 h. Upon completion, the reaction mixture was concentrated to dryness. The residue was purified by flash chromatography on silica eluting with ethyl acetate/heptane mixtures (0/100 to 50/50, v/v) to afford the expected product as a yellow oil (0.64 g, 1.4 mmol, 31% yield). LC-MS (ESI+): 475.1 (M+H⁺).

### Synthesis of Compounds 73 and 74

2-Aminoethane-1-sulfonamide hydrochloride (0.54 g, 3.35 mmol, 2.5 eq.) was added to a stirring solution of methyl (Z)-3-(4-chlorophenyl)-N-((2-methyl-2H-1,2,3-triazol-4-yl)sulfonyl)-4-phenyl-4,5-dihydro-1H-pyrazole-1-carbimidothioate (0.64 g, 1.3 mmol, 73.1) and triethylamine (0.65 mL, 4.7 mmol, 3.5 eq.) in methanol (2 mL). After stirring at room temperature for 18 hours, solvents were removed under reduced pressure. The residue was chromatographed on silica eluting with methanol/dichloromethane mixtures (0/100 to 10/90, v/v) to obtain a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—MeOH+0.1% diethylamine, at 35° C., BPR 100 bar. Acquisition frequency was set to 220 nm for the DAD detector.

Compound 73: (27.6 mg, 0.05 mmol, 4% yield, 97% purity, 99% ee, OR: −50.7). ¹H NMR (DMSO-d₆, 400 MHz) δ 8.30 (br s, 1H), 8.03 (s, 1H), 7.76 (d, J=8.6 Hz, 2H), 7.47 (d, J=8.6 Hz, 2H), 7.30-7.37 (m, 2H), 7.22-7.30 (m, 3H), 7.03 (s, 2H), 5.13 (dd, J=11.2, 4.3 Hz, 1H), 4.57 (t, J=11.2 Hz, 1H), 4.09-4.20 (m, 4H), 3.76 (q, J=6.3 Hz, 2H), 3.25-3.31 (m, 2H) ppm. LC-MS (ESI+): 551.0 (M+H⁺).

Compound 74: (32.4 mg, 0.06 mmol, 4% yield, 94% purity, 99% ee, OR: +94.6). ¹H NMR (DMSO-d₆, 400 MHz) δ 8.30 (br s, 1H), 8.03 (s, 1H), 7.76 (d, J=8.6 Hz, 2H), 7.47 (d, J=8.5 Hz, 2H), 7.31-7.37 (m, 2H), 7.23-7.29 (m, 3H), 7.03 (s, 2H), 5.13 (dd, J=11.2, 4.4 Hz, 1H), 4.57 (t, J=11.2 Hz, 1H), 4.10-4.18 (m, 4H), 3.76 (q, J=6.3 Hz, 2H), 3.30 (br s, 2H) ppm. LC-MS (ESI+): 551.0 (M+H⁺).

### Synthesis of (S)-3-(4-chlorophenyl)-N′-((1-isopropyl-1H-1,2,3-triazol-5-yl)sulfonyl)-4-phenyl-N-(2-sulfamoylethyl)-4,5-dihydro-1H-pyrazole-1-carboximidamide (Compound 75) & (R)-3-(4-chlorophenyl)-N′-((1-isopropyl-1H-1,2,3-triazol-5-yl)sulfonyl)-4-phenyl-N-(2-sulfamoylethyl)-4,5-dihydro-1H-pyrazole-1-carboximidamide (Compound 76)

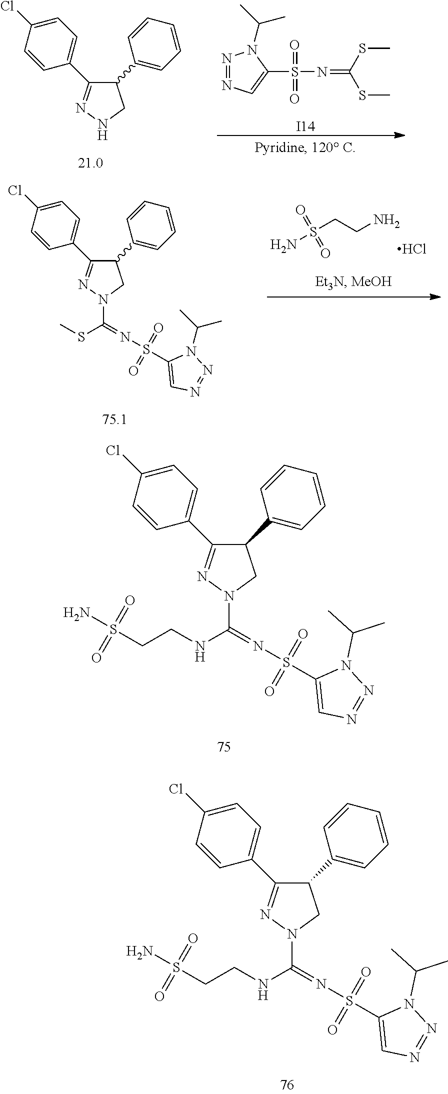

### Synthesis of Methyl 3-(4-chlorophenyl)-N-((1-isopropyl-1H-1,2,3-triazol-5-yl)sulfonyl)-4-phenyl-4,5-dihydro-1H-pyrazole-1-carbimidothioate (75.1)

Dimethyl ((1-isopropyl-1H-1,2,3-triazol-5-yl)sulfonyl)carbonimidodithioate (0.29 g, 0.98 mmol, 114) and 3-(4-chlorophenyl)-4-phenyl-4,5-dihydro-1H-pyrazole (0.25 g, 0.098 mmol, 21.0) in pyridine (3 mL) was refluxed at 120° C. for 18 h. Upon completion, the reaction mixture was concentrated to dryness and the residue was purified by flash chromatography on silica eluting with ethyl acetate/heptane mixtures (0/100 to 50/50, v/v) to afford the expected product as a yellow oil (0.1 g, 0.19 mmol, 20% yield). LC-MS (ESI+): 503.1 (M+H⁺).

### Synthesis of Compounds 75 and 76

2-Aminoethane-1-sulfonamide hydrochloride (78 mg, 0.5 mmol, 2.5 eq.) was added to a stirring solution of methyl 3-(4-chlorophenyl)-N-((1-isopropyl-1H-1,2,3-triazol-5-yl)sulfonyl)-4-phenyl-4,5-dihydro-1H-pyrazole-1-carbimidothioate (98 mg, 0.2 mmol, 75.1) and triethylamine (0.1 mL, 0.7 mmol, 3.5 eq.) in methanol (2 mL). After stirring at room temperature for 18 hours, solvents were removed under reduced pressure. The residue was chromatographed on silica eluting with methanol/dichloromethane mixtures (0/100 to 10/90, v/v) to obtain a yellowish oil, which was further subjected to SFC purification to resolve the two enantiomers as yellowish white solids.

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—MeOH+0.1% diethylamine, at 35° C., BPR 100 bar. Acquisition frequency was set to 220 nm for the DAD detector.

Compound 75: (7.4 mg, 0.013 mmol, 6% yield, 99% purity, 99% ee, OR: −60.9). ¹H NMR (DMSO-d₆, 400 MHz) δ 8.30 (br s, 1H), 8.00 (s, 1H), 7.73 (br d, J=8.3 Hz, 2H), 7.46 (d, J=8.6 Hz, 2H), 7.30-7.37 (m, 2H), 7.21-7.29 (m, 3H), 7.05 (s, 2H), 5.21 (dt, J=13.2, 6.5 Hz, 1H), 5.13 (br dd, J=11.3, 3.6 Hz, 1H), 4.54 (br t, J=11.2 Hz, 1H), 4.03 (br dd, J=11.0, 4.1 Hz, 1H), 3.74 (br s, 2H), 3.26 (br s, 2H), 1.52 (br d, J=6.6 Hz, 6H) ppm. LC-MS (ESI+): 579.1 (M+H⁺).

Compound 76: (9.7 mg, 0.017 mmol, 9% yield, 98% purity, 99% ee, OR: +73.3). ¹H NMR (DMSO-d₆, 400 MHz) δ 8.31 (br s, 1H), 8.00 (s, 1H), 7.73 (d, J=8.5 Hz, 2H), 7.46 (d, J=8.6 Hz, 2H), 7.31-7.37 (m, 2H), 7.22-7.28 (m, 3H), 7.05 (s, 2H), 5.21 (dt, J=13.3, 6.6 Hz, 1H), 5.14 (br dd, J=11.1, 4.3 Hz, 1H), 4.54 (t, J=11.2 Hz, 1H), 4.03 (br dd, J=10.9, 4.2 Hz, 1H), 3.74 (br d, J=5.4 Hz, 2H), 3.28 (br s, 2H), 1.52 (br d, J=6.5 Hz, 6H) ppm. LC-MS (ESI+): 579.1 (M+H⁺).

### Synthesis of (S,Z)-3-(4-chlorophenyl)-N′-((4-chlorophenyl)sulfonyl)-4-(3-cyanophenyl)-N-(2-sulfamoylethyl)-4,5-dihydro-1H-pyrazole-1-carboximidamide (Compound 77) & (R,Z)-3-(4-chlorophenyl)-N′-((4-chlorophenyl)sulfonyl)-4-(3-cyanophenyl)-N-(2-sulfamoylethyl)-4,5-dihydro-1H-pyrazole-1-carboximidamide (Compound 78)

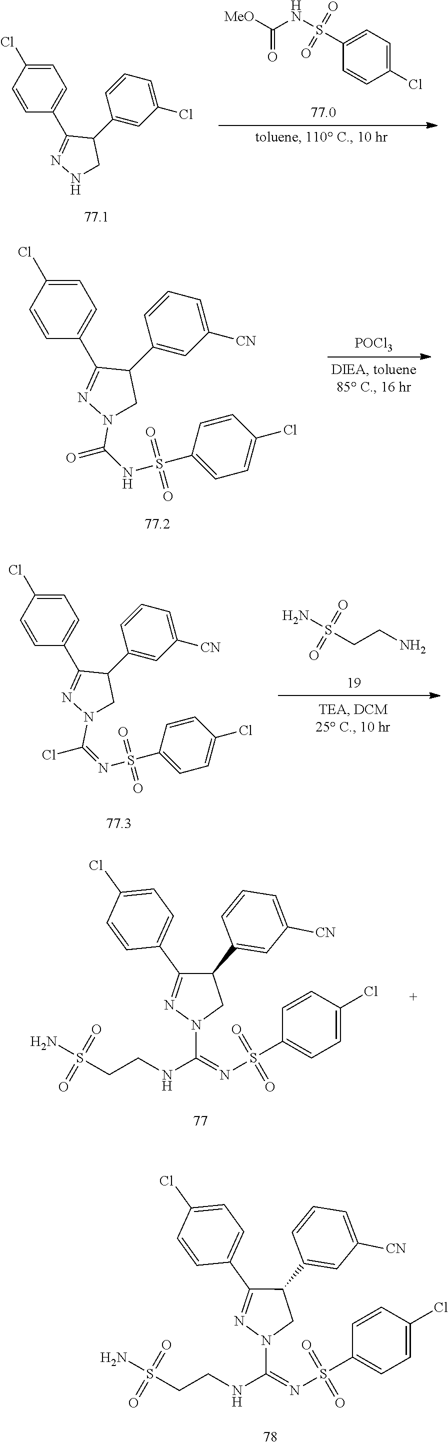

### Synthesis of 77.2

To a solution of 77.1 (300 mg, 1.06 mmol, 1 eq) in toluene (3 mL) at rt was added 77.0 (265.85 mg, 1.06 mmol, CAS #34543-04-9). After stirring at 110° C. for 10 hr, the reaction mixture was concentrated under reduced pressure. The residue was triturated with methyl tertiary butyl ether (10 mL) to give 77.2 (500 mg, 901.14 μmol, 85%) as a white solid. LCMS (ESI+): m/z 499.0 (M+H)+; ¹H NMR: 400 MHz, DMSO-d₆ δ ppm 3.73 (dd, J=11.38, 5.07 Hz, 1H), 4.28 (t, J=11.50 Hz, 1H), 5.08 (dd, J=11.50, 4.95 Hz, 1H), 7.40-7.54 (m, 4H), 7.67-7.82 (m, 6H), 7.94-8.06 (m, 2H), 11.18-11.94 (m, 1H) Synthesis of 77.3

To a solution of 77.2 (300 mg, 600.76 μmol, 1 eq) in toluene (4 mL) at rt were added N, N-diisopropylethylamine (116.47 mg, 901.14 μmol, 156.96 μL, 1.5 eq) and phosphorus oxychloride (72.58 μL, 780.99 μmol, 1.3 eq). After stirring at 85° C. for 16 hr, the reaction mixture was cooled down to rt and concentrated under reduced pressure. The reside was triturated with methyl tertiary butyl ether (10 mL) to give 77.3 (220 mg, 361.13 μmol, 60%) as a yellow solid. LCMS (ESI+): m/z 519.0 (M+H)⁺; ¹H NMR: 400 MHz, DMSO-d₆ δ ppm 4.00 (dd, J=12.53, 5.56 Hz, 1H), 4.65 (br t, J=11.92 Hz, 1H), 5.36 (dd, J=11.25, 5.75 Hz, 1H), 7.46-7.55 (m, 3H), 7.59-7.77 (m, 6H), 7.90-7.94 (m, 2H), 11.62 (br d, J=4.16 Hz, 1H).

### Synthesis of Compounds 77 and 78

To a solution of 77.3 (220 mg, 424.86 μmol, 1 eq) in dichloromethane (3 mL) at rt were added triethylamine (236.54 μL, 1.70 mmol, 4 eq) and 2-aminoethanesulfonamide (79.13 mg, 637.29 μmol, 1.5 eq). After stirring at rt for 12 hr, the reaction mixture was concentrated under reduced pressure. The residue was triturated with methyl tertiary butyl ether (10 mL) to afford racemate (220 mg, 363.33 μmol, 86%) as a white solid. LCMS (ESI+): m/z 605.1 (M+H)⁺; ¹H NMR: 400 MHz, DMSO-d₆ δ ppm 3.28 (br t, J=6.60 Hz, 2H), 3.67-3.80 (m, 2H), 3.99-4.11 (m, 1H), 4.54 (t, J=11.19 Hz, 1H), 5.19 (dd, J=11.19, 4.34 Hz, 1H), 5.76 (s, 1H), 7.03 (s, 2H), 7.41-7.61 (m, 6H), 7.69-7.88 (m, 6H), 8.21 (br t, J=5.01 Hz, 1H) The racemate (200 mg, 330.30 μmol, 1 eq) was separated by SFC separation to obtain Compound 77 (40 mg, 65.53 μmol, 20%) and Compound 78 (34 mg, 56.15 μmol, 17%) as white solids.

Method of the SFC Column: DAICEL CHIRALPAK AD (250 mm*30 mm, 10 μm); Mobile phase A: CO₂; Mobile phase B: IPA (0.1% IPAm, v/v); B %: 55%, Run time: 10 min.

Compound 77: ¹H NMR (400 MHz, DMSO-d₆) δ ppm 3.24-3.31 (m, 2H), 3.66-3.80 (m, 2H), 4.07 (dd, J=11.27, 4.23 Hz, 1H), 4.54 (t, J=11.32 Hz, 1H), 5.14-5.24 (m, 1H), 7.04 (br s, 2H), 7.47 (d, J=8.70 Hz, 2H), 7.51-7.58 (m, 4H), 7.69-7.77 (m, 3H), 7.79-7.87 (m, 3H), 8.22 (br s, 1H); LCMS (ESI+): m/z 605 (M+H)⁺ OR: −143.00.

Compound 78: ¹H NMR (400 MHz, DMSO-d₆) δ ppm 3.29 (s, 2H), 3.74 (brd, J=4.28 Hz, 2H), 4.07 (br dd, J=11.31, 4.22 Hz, 1H), 4.54 (t, J=11.25 Hz, 1H), 5.19 (dd, J=11.13, 4.40 Hz, 1H), 7.03 (s, 1H), 7.44-7.57 (m, 6H), 7.70-7.86 (m, 6H), 8.15-8.26 (m, 1H); LCMS (ESI+): m/z 604.90 (M+H)⁺; OR: 141.40.

### Synthesis of (S,Z)-3-(4-chlorophenyl)-N′-((1-methyl-1H-pyrazol-4-yl)sulfonyl)-4-phenyl-N-(2-sulfamoylethyl)-4,5-dihydro-1H-pyrazole-1-carboximidamide (Compound 81) & (R,Z)-3-(4-chlorophenyl)-N′-((1-methyl-1H-pyrazol-4-yl)sulfonyl)-4-phenyl-N-(2-sulfamoylethyl)-4,5-dihydro-1H-pyrazole-1-carboximidamide (Compound 82)

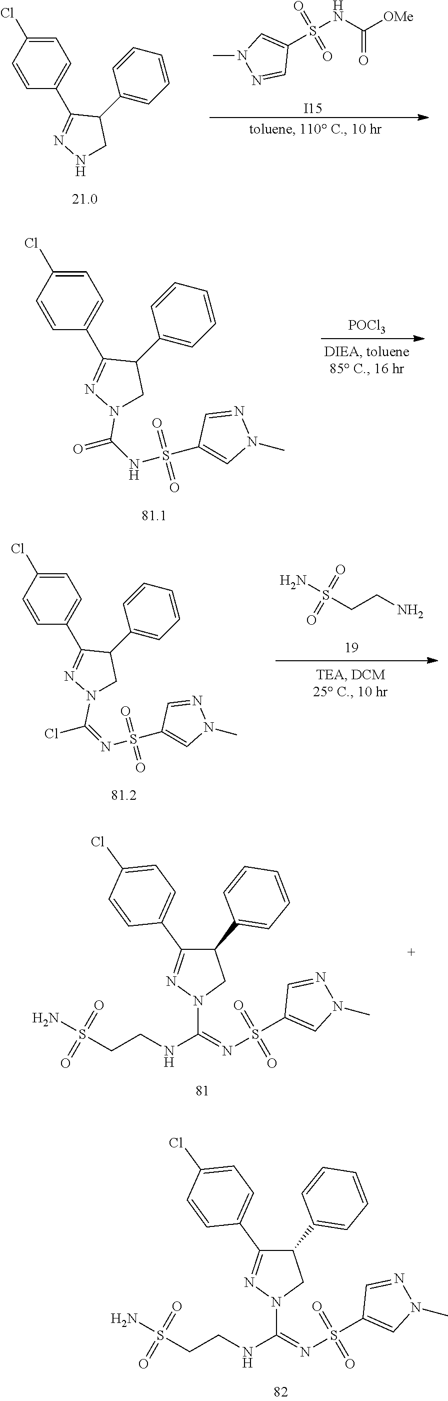

### Synthesis of 81.1

To a solution of 21.0 (157 mg, 716.18 μmol, 1 eq) in toluene (2 mL) at rt was added 115 (183.87 mg, 716.18 μmol, 1 eq). After stirring at 110° C. for 10 hr, the mixture was concentrated under reduced pressure. The residue was triturated with methyl tertiary butyl ether (5 mL) to give 81.1 (127 mg, 257.49 μmol, 36%) as a white solid. LCMS (ESI+): m/z 443.9 (M+H)⁺. ¹H NMR: 400 MHz, DMSO-d₆ δ ppm 3.71 (dd, J=11.21, 4.77 Hz, 1H), 3.90 (s, 3H), 4.30 (t, J=11.38 Hz, 1H), 4.98 (dd, J=11.38, 4.71 Hz, 1H), 7.16-7.25 (m, 3H), 7.30 (br d, J=7.51 Hz, 2H), 7.42 (d, J=8.58 Hz, 2H), 7.78-7.87 (m, 3H), 8.41 (s, 1H), 11.32 (s, 1H).

### Synthesis of 81.2

To a solution of 81.1 (157 mg, 353.68 μmol, 1 eq) in toluene (4 mL) at rt were added N,N-diisopropylethylamine (92.40 μL, 530.52 μmol, 92.40 μL, 1.5 eq) and phosphorus oxychloride (42.73 μL, 459.78 μmol, 1.3 eq). After stirring at 85° C. for 16 hr, the mixture was concentrated under reduced pressure. The residue was triturated with methyl tertiary butyl ether (10 mL) to give 81.2 (400 mg, 809.16 μmol, 77%) as a yellow solid, which was used for the next step without further purification. LCMS (ESI+): m/z 461.9 (M+H)⁺.

### Synthesis of 81 & 82

To a solution of 81.2 (157 mg, 339.57 μmol, 1 eq) in dichloromethane (5 mL) at rt were added triethylamine (236.32 μL, 1.70 mmol, 5 eq) and 2-aminoethanesulfonamide (63.24 mg, 393.72 μmol, 1.16 eq). After stirring at rt for 12 hr, the mixture was concentrated under reduced pressure. The residue was purified by column on silica gel using petroleum ether and ethyl acetate (1/1, v/v) to afford a mixture of enantiomers (60 mg, 109.08 μmol, 32%) as a white solid. LCMS (ESI+): m/z 550.0 (M+H)⁺. ¹H NMR: 400 MHz, DMSO-d₆ δ ppm 3.17 (s, 3H), 3.30 (br s, 1H), 3.80 (br s, 1H), 3.82 (s, 3H), 4.01-4.19 (m, 2H), 4.53 (t, J=11.25 Hz, 1H), 5.08 (dd, J=11.13, 4.28 Hz, 1H), 7.19-7.37 (m, 6H), 7.45 (d, J=8.56 Hz, 2H), 7.69 (s, 1H), 7.74 (d, J=8.56 Hz, 2H), 8.12 (s, 1H). The enantiomers (130 mg, 236.34 μmol, 1 eq) was separated by SFC to obtain Compound 81 (15 mg, 27.27 μmol, 12%) and Compound 82 (15 mg, 27.27 μmol, 12%) as two white solids.

Method of the SFC Column: DAICEL CHIRALPAK AD (250 mm*30 mm, 10 μm); Mobile phase A: CO₂; Mobile phase B: IPA (0.1% IPAm, v/v); B %: 55%, Run time: 10 min.

Compound 81: ¹H NMR (400 MHz, DMSO-d₆) δ ppm 3.30 (brs, 2H), 3.72-3.87 (m, 5H), 4.13 (br dd, J=10.76, 4.03 Hz, 1H), 4.54 (br t, J=11.25 Hz, 1H), 5.09 (dd, J=11.31, 4.34 Hz, 1H), 7.05 (s, 2H), 7.21-7.29 (m, 3H), 7.32 (br d, J=7.34 Hz, 2H), 7.46 (d, J=8.56 Hz, 2H), 7.69 (s, 1H), 7.75 (d, J=8.56 Hz, 2H), 8.04 (br s, 1H), 8.13 (s, 1H) LCMS (ESI+): m/z 550.0 (M+H)⁺OR: −111.80.

Compound 82: ¹H NMR (400 MHz, DMSO-d₆) δ ppm 3.29 (s, 2H), 3.72-3.90 (m, 5H), 4.05-4.19 (m, 1H), 4.54 (t, J=11.25 Hz, 1H), 5.08 (dd, J=11.25, 4.40 Hz, 1H), 7.05 (s, 2H), 7.21-7.29 (m, 3H), 7.30-7.37 (m, 2H), 7.43-7.49 (m, 2H), 7.69 (s, 1H), 7.72-7.80 (m, 2H), 8.03 (br t, J=5.07 Hz, 1H), 8.13 (s, 1H). LCMS (ESI+): m/z 550.0 (M+H)⁺. OR: 118.20.

### Synthesis of (S,E)-3-(4-methoxyphenyl)-4-phenyl-N-(2-sulfamoylethyl)-N′-((4-(trifluoromethyl)phenyl)sulfonyl)-4,5-dihydro-1H-pyrazole-1-carboximidamide (Compound 83) & (S,E)-3-(4-methoxyphenyl)-4-phenyl-N-(2-sulfamoylethyl)-N′-((4-(trifluoromethyl)phenyl)sulfonyl)-4,5-dihydro-1H-pyrazole-1-carboximidamide (Compound 84)

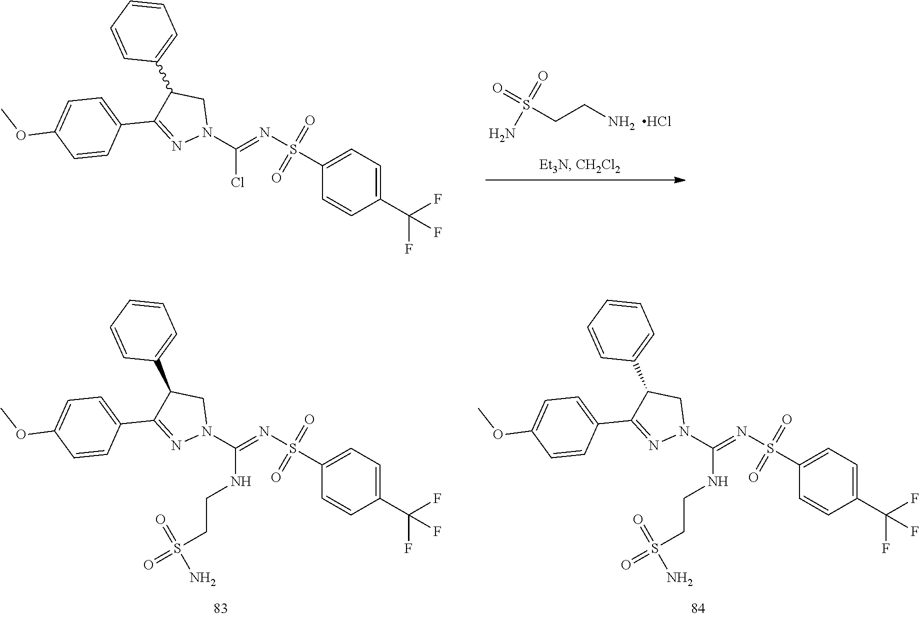

To a solution of (S)-2-aminopropanamide hydrochloride (0.085 g, 0.53 mmol, 1.1 eq.) and triethylamine (0.2 mL, 1.4 mmol, 3 eq.) in dichloromethane (5 mL) was added (Z)-3-(4-methoxyphenyl)-4-phenyl-N-((4-(trifluoromethyl)phenyl)sulfonyl)-4,5-dihydro-1H-pyrazole-1-carbimidoyl chloride (0.25 mg, 0.5 mmol, 1 eq.). After stirring at room temperature for 18 hours, the reaction was diluted with dichloromethane (20 mL) and washed with 2N HCl (15 mL). The organic layer was dried over magnesium sulfate, filtered, and concentrated in vacuo. The residue was purified by flash chromatography on silica eluting with methanol/dichloromethane mixtures (0/100 to 10/90) to give the expected compound as a colorless oil. This racemic compound was further subjected to SFC separate enantiomers

SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 83: (103.5 mg, 0.17 mmol, 36% yield, 99% purity, 99% ee). ¹H NMR (400 MHz, DMSO-d₆) δ 8.18 (br d, J=6.1 Hz, 1H), 8.03 (d, J=8.1 Hz, 2H), 7.84 (d, J=8.4 Hz, 2H), 7.65 (d, J=8.7 Hz, 2H), 7.3-7.4 (m, 2H), 7.2-7.3 (m, 3H), 7.03 (br s, 2H), 6.92 (d, J=8.9 Hz, 2H), 5.06 (dd, J=4.1, 10.9 Hz, 1H), 4.49 (t, J=11.1 Hz, 1H), 4.02 (dd, J=4.0, 11.2 Hz, 1H), 3.75 (s, 3H), 3.72 (br s, 2H), 3.29 (br d, J=7.2 Hz, 2H). LC-MS (ESI+): 610.2 (M+H⁺).

Compound 84: (103.3 mg, 0.17 mmol, 36% yield, 99% purity, 99% ee). ¹H NMR (400 MHz, DMSO-d₆) δ 8.16 (br s, 1H), 8.02 (d, J=8.2 Hz, 2H), 7.83 (d, J=8.4 Hz, 2H), 7.64 (br d, J=8.7 Hz, 2H), 7.3-7.4 (m, 2H), 7.2-7.3 (m, 3H), 7.02 (br s, 2H), 6.91 (d, J=8.9 Hz, 2H), 5.05 (dd, J=4.0, 11.0 Hz, 1H), 4.48 (t, J=11.2 Hz, 1H), 4.01 (dd, J=4.0, 11.2 Hz, 1H), 3.74 (s, 3H), 3.71 (br s, 2H), 3.3 (m, 3H). LC-MS (ESI+): 610.2 (M+H⁺).

### Synthesis of (S,Z)-3-(4-chlorophenyl)-N′-((1-methyl-1H-pyrazol-3-yl)sulfonyl)-4-phenyl-N-(2-sulfamoylethyl)-4,5-dihydro-1H-pyrazole-1-carboximidamide (Compound 85) & (R,Z)-3-(4-chlorophenyl)-N′-((1-methyl-1H-pyrazol-3-yl)sulfonyl)-4-phenyl-N-(2-sulfamoylethyl)-4,5-dihydro-1H-pyrazole-1-carboximidamide (Compound 86)

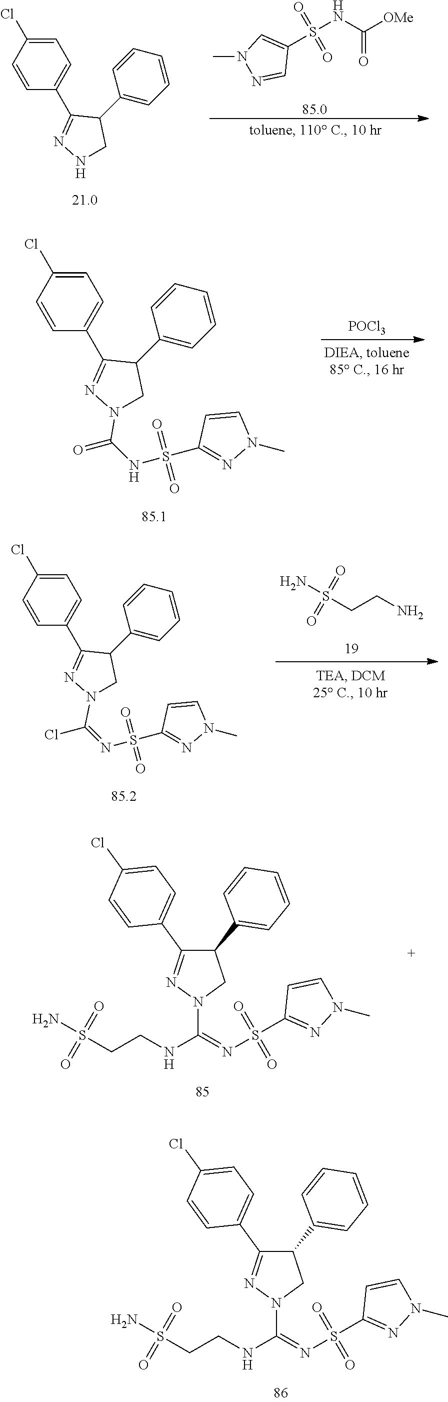

### Synthesis of 85.1

To a solution of 21.0 (365 mg, 1.67 mmol, 1 eq) in toluene (3 mL) at rt was added methyl ((1-methyl-1H-pyrazol-3-yl)sulfonyl)carbamate (427.46 mg, 1.67 mmol, 1 eq). After stirring at 110 0C for 10 hr, the mixture was concentrated under reduced pressure. The residue was triturated with methyl tertiary butyl ether (5 mL) to give 85.1 (300 mg, 608.24 μmol, 37%) as a white solid. LCMS (ESI+): m/z 443.9 (M+H)⁺. ¹H NMR: 400 MHz, DMSO-d₆ δ ppm 3.69 (dd, J=11.37, 4.65 Hz, 1H), 3.94 (s, 3H), 4.27 (t, J=11.37 Hz, 1H), 4.98 (dd, J=11.43, 4.71 Hz, 1H), 6.71-6.81 (m, 1H), 7.16-7.26 (m, 2H), 7.28-7.36 (m, 2H), 7.42 (d, J=8.68 Hz, 2H), 7.84 (d, J=8.68 Hz, 2H), 7.87-7.91 (m, 1H), 11.54 (br s, 1H), 12.09 (br s, 1H).

### Synthesis of 85.2

To a solution of 85.1 (300 mg, 675.82 μmol, 1 eq) in toluene (7 mL) at rt were added N, N-diisopropylethylamine (176.57 μL, 1.01 mmol, 1.5 eq) and phosphorus oxychloride (134.71 mg, 878.56 μmol, 81.64 μL, 1.3 eq). After stirring at 85° C. for 16 hr, the mixture was concentrated under reduced pressure. The residue was triturated with methyl tertiary butyl ether (10 mL) to give 85.2 (300 mg, 551.53 μmol, 82%) as a yellow solid, which was used for the next step without further purification. LCMS (ESI+): m/z 461.9 (M+H)⁺.

### Synthesis of Compounds 85 and 86

To a solution of 85.2 (300 mg, 648.86 μmol, 1 eq) in dichloromethane (3 mL) at rt were added triethylamine (451.56 μL,3.24 mmol, 5 eq) and 2-aminoethanesulfonamide (80.56 mg, 648.86 μmol, 1 eq). After stirring at rt for 12 hr, the mixture was concentrated under reduced pressure. The residue was purified by column on silica gel using petroleum ether and ethyl acetate (1/1, v/v) to give racemate (30 mg, 51.81 μmol, 8%) as a white solid. LCMS (ESI+): m/z 550.0 (M+H)⁺. Racemate (30 mg, 51.81 μmol, 8%) was separated by SFC to obtain Compound 85 (5 mg, 9.09 μmol, 1.40%) and Compound 86 (7 mg, 12.73 μmol, 2%) as white solids.

Method of the SFC: Column: DAICEL CHIRALPAK AD (250 mm*30 mm, 10 μm); Mobile phase A: CO₂; Mobile phase B: IPA (0.1% IPAm, v/v); B %: 55%, Run time: 10 min.

Compound 85: ¹H NMR (400 MHz, DMSO-d₆) δ ppm 3.26-3.31 (m, 2H), 3.74-3.88 (m, 5H), 4.13 (br dd, J=10.57, 3.81 Hz, 1H), 4.54 (br t, J=11.32 Hz, 1H), 5.09 (dd, J=11.19, 4.32 Hz, 1H), 6.51 (d, J=2.25 Hz, 1H), 7.02 (s, 2H), 7.21-7.37 (m, 5H), 7.46 (d, J=8.63 Hz, 2H), 7.69-7.81 (m, 3H), 8.15 (br s, 1H); LCMS (ESI+): m/z 550.0 (M+H)⁺.

Compound 86: ¹H NMR (400 MHz, DMSO-d₆) δ ppm 3.27 (brs, 2H), 3.81 (s, 5H), 4.00-4.18 (m, 1H), 4.49 (br t, J=11.01 Hz, 1H), 5.04 (br dd, J=10.57, 3.69 Hz, 1H), 6.49 (d, J=2.13 Hz, 1H), 7.20-7.35 (m, 6H), 7.45 (d, J=8.63 Hz, 2H), 7.69-7.76 (m, 3H); LCMS (ESI+): m/z 550.0 (M+H)⁺.

### Synthesis of (S,E)-3-(4-chlorophenyl)-N′-((4-chlorophenyl)sulfonyl)-N-(2,2-dimethyl-3-sulfamoylpropyl)-4-phenyl-4,5-dihydro-1H-pyrazole-1-carboximidamide (Compound 87) & (R,E)-3-(4-chlorophenyl)-N′-((4-chlorophenyl)sulfonyl)-N-(2,2-dimethyl-3-sulfamoylpropyl)-4-phenyl-4,5-dihydro-1H-pyrazole-1-carboximidamide (Compound 88)

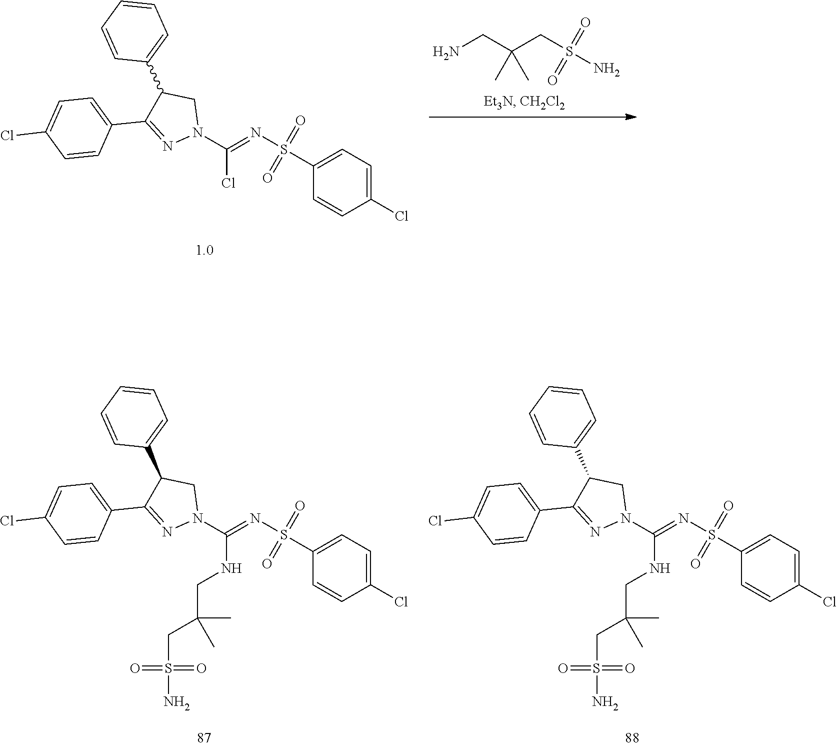

### Synthesis of Compounds 87 and 88

To a solution of 3-amino-2,2-dimethylpropane-1-sulfonamide (126 mg, 0.76 mmol, 1.5 eq.) and triethylamine (0.21 mL, 1.5 mmol, 3.0 eq.) in dichloromethane (3 mL) was added (E)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0). After stirring at rt for 18 h, solvents were removed under reduced pressure. The residue was purified by flash chromatography on silica eluting with ethyl acetate/heptane mixtures (0/100 to 50/50, v/v) to give a colorless oil, which was further subjected to SFC separation conditions, obtaining the following enantiomers as white solids: Compound 87 (85.4 mg, 0.14 mmol, 27% yield, 99% purity, 99% ee, OR: −84.9) and Compound 88 (80.6 mg, 0.13 mol, 26% yield, 99% purity, 99% ee, OR: +77.1).

Method of the SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO₂ (40%)—EtOH+0.1% diethylamine, at 35° C., BPR 100 bar. Acquisition frequency was set to 220 nm for the DAD detector.

Compound 87: ¹H NMR (DMSO-d₆, 400 MHz) δ 7.85 (br s, 1H), 7.80 (d, J=8.6 Hz, 2H), 7.73 (d, J=8.7 Hz, 2H), 7.56 (d, J=8.6 Hz, 2H), 7.45 (d, J=8.6 Hz, 2H), 7.31-7.37 (m, 2H), 7.22-7.29 (m, 3H), 6.98 (s, 2H), 5.10 (dd, J=11.1, 4.1 Hz, 1H), 4.57 (t, J=11.2 Hz, 1H), 4.08 (br dd, J=11.0, 3.8 Hz, 1H), 3.37 (br s, 1H), 3.30 (s, 1H), 3.03 (s, 2H), 1.01 (s, 6H) ppm. LC-MS (ESI+): 622.0 (M+H⁺).

Compound 88: ¹H NMR (DMSO-d₆, 400 MHz) δ 7.85 (br s, 1H), 7.80 (d, J=8.6 Hz, 2H), 7.73 (d, J=8.6 Hz, 2H), 7.56 (d, J=8.7 Hz, 2H), 7.45 (d, J=8.7 Hz, 2H), 7.31-7.37 (m, 2H), 7.21-7.29 (m, 3H), 6.98 (s, 2H), 5.10 (dd, J=11.1, 4.1 Hz, 1H), 4.57 (t, J=11.2 Hz, 1H), 4.08 (br dd, J=11.1, 3.8 Hz, 1H), 3.43 (br s, 1H), 3.29 (s, 1H), 3.03 (s, 2H), 1.01 (s, 6H) ppm. LC-MS (ESI+): 622.0 (M+H⁺).

### Synthesis of (S,E)-N′-((4-chlorophenyl)sulfonyl)-N-(2,2-dimethyl-3-sulfamoylpropyl)-3-(4-fluorophenyl)-4-phenyl-4,5-dihydro-1H-pyrazole-1-carboximidamide (Compound 89) & (R,E)-N′-((4-chlorophenyl)sulfonyl)-N-(2,2-dimethyl-3-sulfamoylpropyl)-3-(4-fluorophenyl)-4-phenyl-4,5-dihydro-1H-pyrazole-1-carboximidamide (Compound 90)

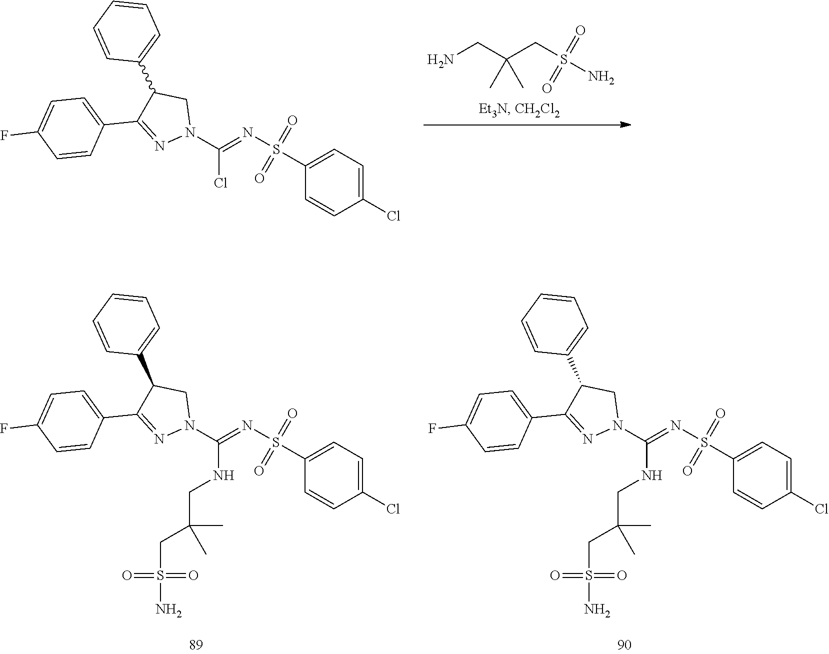

### Synthesis of Compounds 89 and 90

To a solution of 3-amino-2,2-dimethylpropane-1-sulfonamide (121 mg, 0.72 mmol, 1.5 eq.) and triethylamine (0.2 mL, 1.5 mmol, 3.0 eq.) in dichloromethane (3 mL) was added 4-Chloro-N-{chloro-[3-(4-fluoro-phenyl)-4-phenyl-4,5-dihydro-pyrazol-1-yl]-methylene}-benzenesulfonamide (230 mg, 0.48 mmol, 1.0 eq.). After stirring at rt for 18 h, solvents were removed under reduced pressure. The residue was purified by flash chromatography on silica eluting with ethyl acetate/heptane mixtures (0/100 to 60/40, v/v) to give a colorless oil, which was further subjected to SFC separation conditions, obtaining the following 2 different enantiomers as white solids: Compound 89 (71.5 mg, 0.12 mmol, 25% yield, 99% purity, 99% ee, OR: −130.5) and Compound 90 (73.4 mg, 0.12 mol, 25% yield, 99% purity, 99% ee, OR: +98.5).

Method of the SFC: SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%)—MeOH+0.1% diethylamine, at 35° C., BPR 100 bar. Acquisition frequency was set to 220 nm for the DAD detector.

Compound 89: ¹H NMR (DMSO-d₆, 400 MHz) δ 7.74-7.88 (m, 5H), 7.53-7.59 (m, 2H), 7.31-7.37 (m, 2H), 7.20-7.29 (m, 5H), 6.98 (s, 2H), 5.10 (br dd, J=11.0, 4.1 Hz, 1H), 4.56 (br t, J=11.1 Hz, 1H), 4.08 (br dd, J=11.0, 3.7 Hz, 1H), 3.30 (s, 2H), 3.03 (s, 2H), 1.01 (s, 6H) ppm. LC-MS (ESI+): 606.1 (M+H+).

Compound 90: ¹H NMR (DMSO-d₆, 400 MHz) δ 7.74-7.85 (m, 5H), 7.56 (d, J=8.7 Hz, 2H), 7.30-7.37 (m, 2H), 7.19-7.29 (m, 5H), 6.98 (br s, 2H), 5.10 (br dd, J=11.2, 4.1 Hz, 1H), 4.56 (t, J=11.1 Hz, 1H), 4.07 (br dd, J=10.9, 3.6 Hz, 1H), 3.30 (s, 2H), 3.03 (s, 2H), 1.01 (s, 6H) ppm. LC-MS (ESI+): 606.1 (M+H⁺).

### Synthesis of (S,E)-3-(4-chlorophenyl)-N′-((4-chlorophenyl)sulfonyl)-N-((S)-2-methyl-3-sulfamoylpropyl)-4-phenyl-4,5-dihydro-1H-pyrazole-1-carboximidamide (Compound 91), (S,E)-3-(4-chlorophenyl)-N′-((4-chlorophenyl)sulfonyl)-N-((R)-2-methyl-3-sulfamoylpropyl)-4-phenyl-4,5-dihydro-1H-pyrazole-1-carboximidamide (Compound 92), (R,E)-3-(4-chlorophenyl)-N′-((4-chlorophenyl)sulfonyl)-N-((S)-2-methyl-3-sulfamoylpropyl)-4-phenyl-4,5-dihydro-1H-pyrazole-1-carboximidamide (Compound 93) & (R,E)-3-(4-chlorophenyl)-N′-((4-chlorophenyl)sulfonyl)-N-((R)-2-methyl-3-sulfamoylpropyl)-4-phenyl-4,5-dihydro-1H-pyrazole-1-carboximidamide (Compound 94)

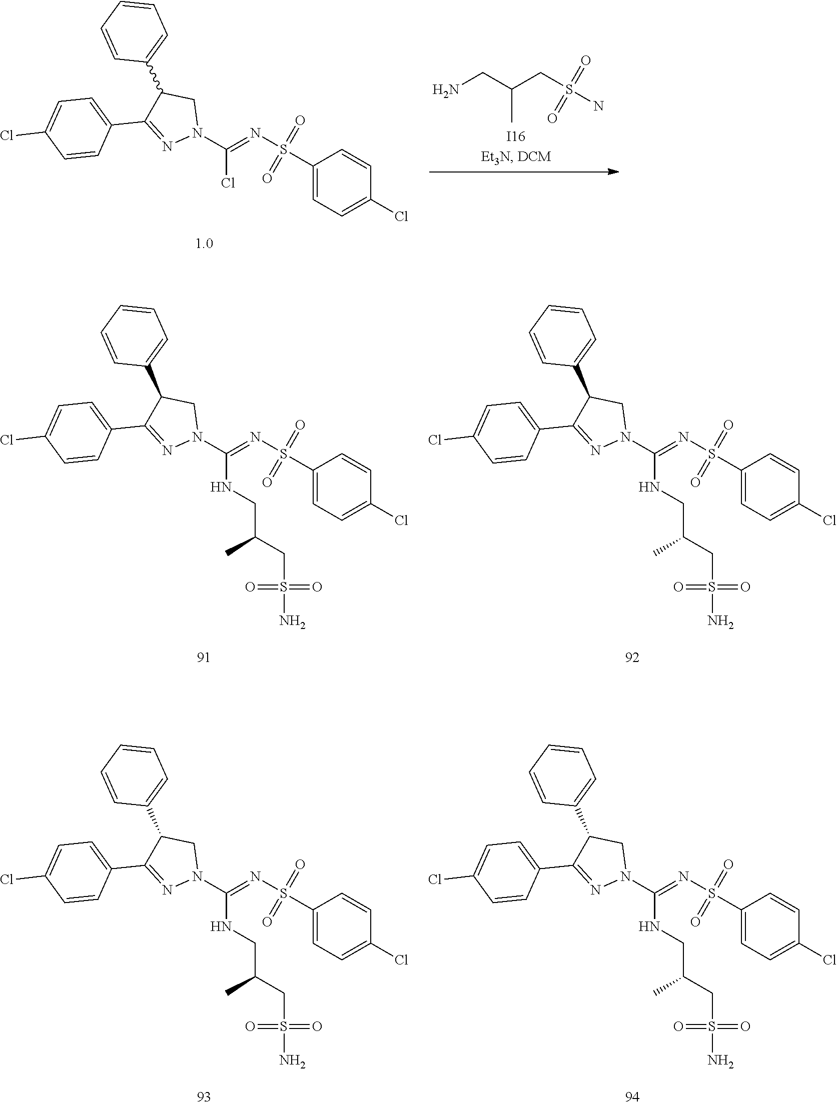

### Synthesis of Compounds 91-94

To a stirred solution of 3-amino-2-methylpropane-1-sulfonamide (185 mg, 1.22 mmol, 2.0 eq., 116) and triethylamine (0.26 mL, 1.83 mmol, 3.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (300 mg, 0.61 mmol, 1.0). After stirring at rt for 18 h, solvents were removed under reduced pressure. The residue was chromatographed on silica eluting with ethyl acetate/heptane mixtures (0/100 to 60/40) to obtain a colorless oil, which was further subjected to SFC purification to resolve the four diastereomers as white solids: Compound 91 (9.7 mg, 0.016 mmol, % yield, 99% purity, 99% ee, OR: −50.9), Compound 92 (8.1 mg, 0.013 mmol, % yield, 99% purity, 99% ee, OR: −59.2), Compound 93 (8.3 mg, 0.013 mmol, % yield, 99% purity, 99% ee, OR: +29.0), and Compound 94 (12.3 mg, 0.020 mmol, % yield, 98% purity, 99% ee, OR: +41.7).

Method of the SFC: SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector. Absolute stereochemistry was not determined. Diastereomers with (-) OR were arbitrary assigned as S isomer.

Compound 91: ¹H NMR (MeOD-d₄, 400 MHz) δ 7.85 (d, J=8.6 Hz, 2H), 7.69 (d, J=8.6 Hz, 2H), 7.48 (d, J=8.6 Hz, 2H), 7.17-7.37 (m, 7H), 4.94 (dd, J=11.3, 4.7 Hz, 1H), 4.54 (t, J=11.3 Hz, 1H), 4.10 (dd, J=11.3, 4.7 Hz, 1H), 3.41-3.60 (m, 2H), 3.23 (dd, J=14.3, 5.0 Hz, 1H), 2.98 (dd, J=14.3, 7.4 Hz, 1H), 2.37-2.52 (m, 1H), 1.10 (d, J=6.9 Hz, 3H) ppm. LC-MS (ESI+): 608.0 (M+H+).

Compound 92: ¹H NMR (MeOD-d₄, 400 MHz) δ 7.85 (d, J=8.5 Hz, 2H), 7.70 (d, J=8.6 Hz, 2H), 7.48 (d, J=8.6 Hz, 2H), 7.17-7.37 (m, 7H), 4.94 (dd, J=11.2, 4.6 Hz, 1H), 4.55 (t, J=11.3 Hz, 1H), 4.09 (dd, J=11.2, 4.6 Hz, 1H), 3.50 (br d, J=6.1 Hz, 2H), 3.23 (dd, J=14.3, 4.9 Hz, 1H), 2.97 (dd, J=14.3, 7.3 Hz, 1H), 2.38-2.51 (m, 1H), 1.11 (d, J=6.8 Hz, 3H) ppm. LC-MS (ESI+): 608.0 (M+H+).

Compound 93: ¹H NMR (MeOD-d₄, 400 MHz) δ 7.85 (d, J=8.6 Hz, 2H), 7.69 (d, J=8.6 Hz, 2H), 7.48 (d, J=8.6 Hz, 2H), 7.17-7.37 (m, 7H), 4.94 (dd, J=11.2, 4.7 Hz, 1H), 4.54 (t, J=11.3 Hz, 1H), 4.10 (dd, J=11.3, 4.7 Hz, 1H), 3.41-3.59 (m, 2H), 3.23 (dd, J=14.3, 4.9 Hz, 1H), 2.98 (dd, J=14.3, 7.4 Hz, 1H), 2.37-2.51 (m, 1H), 1.10 (d, J=6.9 Hz, 3H) ppm. LC-MS (ESI+): 608.0 (M+H+).

Compound 94: ¹H NMR (MeOD-d₄, 400 MHz) δ 7.85 (d, J=8.6 Hz, 2H), 7.70 (d, J=8.6 Hz, 2H), 7.48 (d, J=8.6 Hz, 2H), 7.16-7.38 (m, 7H), 4.94 (dd, J=11.2, 4.6 Hz, 1H), 4.55 (t, J=11.3 Hz, 1H), 4.08 (dd, J=11.3, 4.6 Hz, 1H), 3.50 (br d, J=6.2 Hz, 2H), 3.23 (dd, J=14.3, 4.9 Hz, 1H), 2.97 (dd, J=14.3, 7.4 Hz, 1H), 2.45 (br dd, J=12.2, 6.7 Hz, 1H), 1.10 (d, J=6.8 Hz, 3H) ppm. LC-MS (ESI+): 608.0 (M+H+).

### Synthesis of (S,E)-3-(4-chlorophenyl)-N′-((4-chlorophenyl)sulfonyl)-4-phenyl-N-((R)-3-sulfamoylbutyl)-4,5-dihydro-1H-pyrazole-1-carboximidamide (Compound 95), (S,E)-3-(4-chlorophenyl)-N′-((4-chlorophenyl)sulfonyl)-4-phenyl-N-((S)-3-sulfamoylbutyl)-4,5-dihydro-1H-pyrazole-1-carboximidamide (Compound 96), (R,E)-3-(4-chlorophenyl)-N′-((4-chlorophenyl)sulfonyl)-4-phenyl-N-((R)-3-sulfamoylbutyl)-4,5-dihydro-1H-pyrazole-1-carboximidamide (Compound 97), & (R,E)-3-(4-chlorophenyl)-N′-((4-chlorophenyl)sulfonyl)-4-phenyl-N-((S)-3-sulfamoylbutyl)-4,5-dihydro-1H-pyrazole-1-carboximidamide (Compound 98)

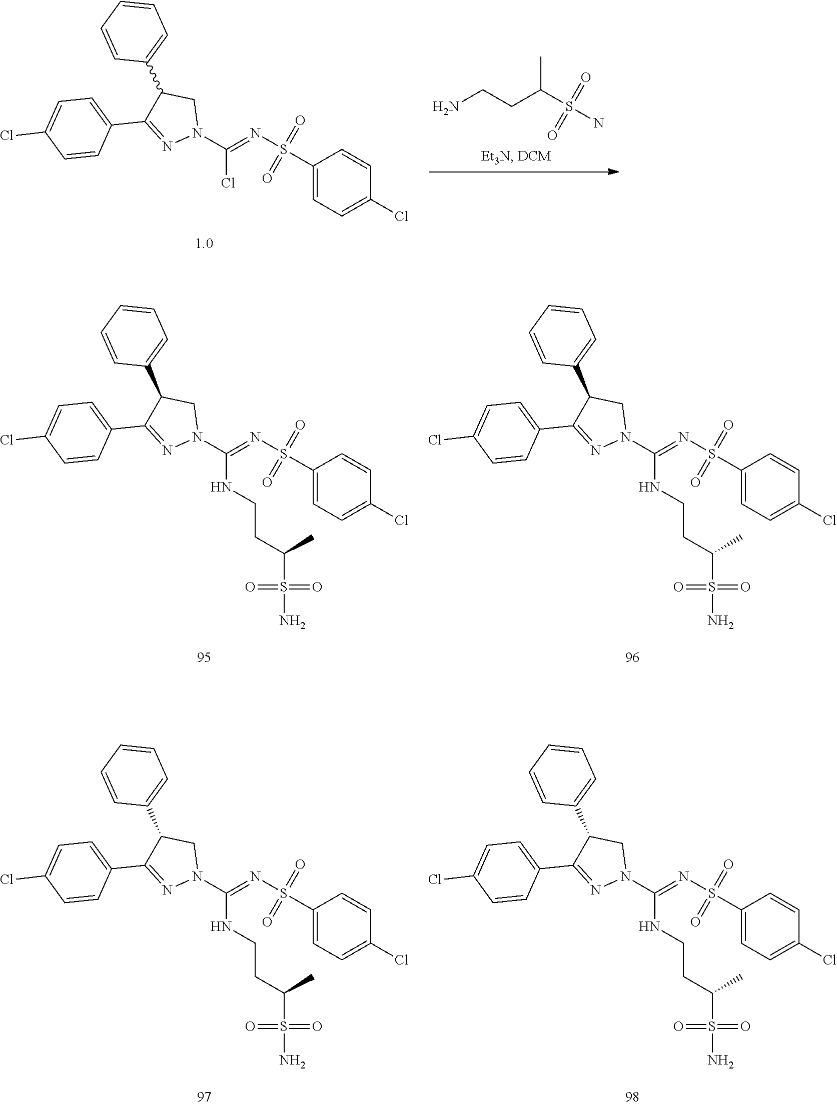

### Synthesis of Compounds 95-98

To a stirred solution of 4-aminobutane-2-sulfonamide (185 mg, 1.22 mmol, 2.0 eq.) and triethylamine (0.26 mL, 1.83 mmol, 3.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (300 mg, 0.61 mmol, 1.0). After stirring at rt for 18 h, solvents were removed under reduced pressure. The residue was chromatographed on silica eluting with ethyl acetate/heptane mixtures (0/100 to 60/40) to obtain a colorless oil, which was further subjected to SFC purification to resolve the four diastereomers as white solids: Compound 95 (14.2 mg, 0.023 mmol, 4% yield, 99% purity, 99% ee, OR: −33.9), Compound 96 (11.7 mg, 0.019 mmol, 3% yield, 99% purity, 99% ee, OR: −59.1), Compound 97 (6.0 mg, 0.010 mmol, 2% yield, 99% purity, 99% ee, OR: +19.1), and Compound 98 (12.6 mg, 0.021 mmol, 4% yield, 99% purity, 99% ee, OR: +78.1).

Method of the SFC: SFC purification was performed on a Jasco 4000 Parallel SFC System (4× Channels 3×UV+1×DAD) using an Phenomenex column (Lux Amylose-3) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector. Absolute stereochemistry was not determined. Diastereomers with (-) OR were arbitrary assigned as S isomer.

Compound 95: ¹H NMR (MeOD-d₄, 400 MHz) δ 7.85 (d, J=8.5 Hz, 2H), 7.69 (d, J=8.6 Hz, 2H), 7.47 (d, J=8.5 Hz, 2H), 7.14-7.37 (m, 7H), 4.93 (dd, J=11.2, 4.6 Hz, 1H), 4.53 (t, J=11.3 Hz, 1H), 4.08 (dd, J=11.5, 5.0 Hz, 1H), 3.62 (br t, J=7.1 Hz, 2H), 2.97-3.10 (m, 1H), 2.22-2.34 (m, 1H), 1.79 (dq, J=14.3, 7.1 Hz, 1H), 1.31 (d, J=6.8 Hz, 3H) ppm. LC-MS (ESI+): 608.0 (M+H⁺).

Compound 96 ¹H NMR (MeOD-d₄, 400 MHz) δ 7.85 (d, J=8.6 Hz, 2H), 7.70 (d, J=8.6 Hz, 2H), 7.48 (d, J=8.6 Hz, 2H), 7.17-7.35 (m, 7H), 4.93 (dd, J=11.2, 4.6 Hz, 1H), 4.54 (t, J=11.3 Hz, 1H), 4.07 (dd, J=11.3, 4.6 Hz, 1H), 3.50-3.72 (m, 2H), 2.98-3.10 (m, 1H), 2.21-2.36 (m, 1H), 1.73-1.86 (m, 1H), 1.32 (d, J=6.8 Hz, 3H) ppm. LC-MS (ESI+): 608.1 (M+H⁺).

Compound 97: ¹H NMR (MeOD-d₄, 400 MHz) δ 7.82-7.88 (m, 2H), 7.70 (d, J=8.4 Hz, 2H), 7.48 (dd, J=8.7, 2.0 Hz, 2H), 7.16-7.36 (m, 7H), 4.90-4.97 (m, 1H), 4.54 (td, J=11.3, 6.0 Hz, 1H), 4.04-4.12 (m, 1H), 3.52-3.70 (m, 2H), 2.98-3.09 (m, 1H), 2.22-2.34 (m, 1H), 1.74-1.85 (m, 1H), 1.32 (d, J=6.8 Hz, 3H) ppm. LC-MS (ESI+): 608.0 (M+H⁺).

Compound 98: ¹H NMR (MeOD-d₄, 400 MHz) δ 7.85 (d, J=8.6 Hz, 2H), 7.70 (d, J=8.7 Hz, 2H), 7.48 (d, J=8.5 Hz, 2H), 7.18-7.35 (m, 7H), 4.93 (dd, J=11.2, 4.6 Hz, 1H), 4.54 (t, J=11.3 Hz, 1H), 4.07 (dd, J=11.3, 4.6 Hz, 1H), 3.52-3.73 (m, 2H), 2.98-3.09 (m, 1H), 2.24-2.35 (m, 1H), 1.74-1.86 (m, 1H), 1.32 (d, J=6.9 Hz, 3H) ppm. LC-MS (ESI+): 608.1 (M+H⁺).

### Synthesis of (S,E)-3-(4-chlorophenyl)-N′-((4-chlorophenyl)sulfonyl)-4-phenyl-N-(4-sulfamoylbutyl)-4,5-dihydro-1H-pyrazole-1-carboximidamide (Compound 99) & (S,E)-3-(4-chlorophenyl)-N′-((4-chlorophenyl)sulfonyl)-4-phenyl-N-(4-sulfamoylbutyl)-4,5-dihydro-1H-pyrazole-1-carboximidamide (Compound 100)

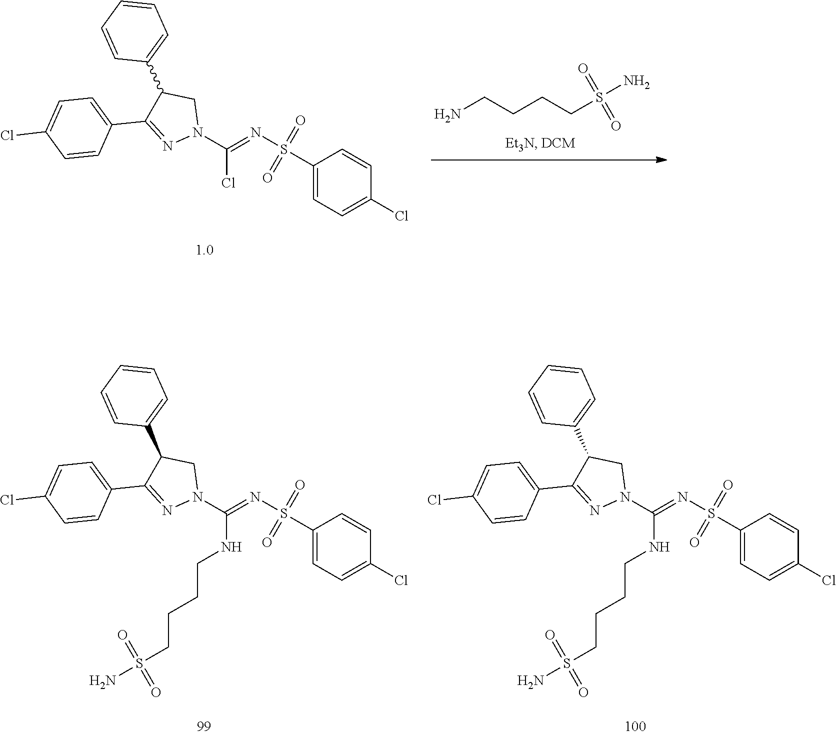 [SMILES: O=S(=O)(/N=C(\Cl)N1CC(c2ccccc2)C(c2ccc(Cl)cc2)=N1)c1ccc(Cl)cc1 ; NCCCCS(N)(=O)=O ; NS(=O)(=O)CCCCN/C(=N\S(=O)(=O)c1ccc(Cl)cc1)N1C[C@H](c2ccccc2)C(c2ccc(Cl)cc2)=N1 ; NS(=O)(=O)CCCCN/C(=N\S(=O)(=O)c1ccc(Cl)cc1)N1C[C@@H](c2ccccc2)C(c2ccc(Cl)cc2)=N1]

### Synthesis of Compounds 99 and 100

To a stirred solution of 4-aminobutane-1-sulfonamide (85 mg, 0.56 mmol, 1.1 eq.) and triethylamine (0.21 mL, 1.5 mmol, 3.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0). After stirring at rt for 18 h, the reaction mixture was diluted with dichloromethane (15 mL) and washed with ammonium chloride (20 mL) and water (20 mL). The organic extract was dried over magnesium sulfate, filtered, and concentrated in vacuo. The residue was purified by flash chromatography on silica eluting with methanol/dichloromethane mixtures (0/100 to 10/90, v/v), affording a colorless oil which was further subjected to SFC separation conditions: Compound 99 (13.6 mg, 0.022 mmol, 5% yield, 98% purity, 99% ee, OR: −60.7) and Compound 100 (13.4 mg, 0.022 mmol, 5% yield, 99% purity, 99% ee, OR: +60.1).

Method of the SFC: SFC purification was performed on a Jasco 4000 Parallel SFC System (4× Channels 3×UV+1×DAD) using an Phenomenex column (Lux Amylose-1) 150 mm long x4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 99: (13.6 mg, 0.022 mmol, 5% yield, 98% purity, 99% ee, OR: −60.7). ¹H NMR (CDCl₃, 400 MHz) δ 7.80-7.90 (m, 2H), 7.53 (br d, J=5.7 Hz, 2H), 7.42 (br d, J=4.9 Hz, 2H), 7.33 (br d, J=4.6 Hz, 5H), 7.13 (br s, 2H), 6.98-7.09 (m, 1H), 4.76 (br s, 2H), 4.62-4.71 (m, 1H), 4.49-4.61 (m, 1H), 4.01-4.18 (m, 1H), 3.63-3.79 (m, 2H), 3.18 (br d, J=7.1 Hz, 2H), 2.02 (br d, J=5.3 Hz, 2H), 1.83-1.94 (m, 2H) ppm. LC-MS (ESI+): 608.0 (M+H+); HPLC RT: 3.867 min (Method: VILLA).

Compound 100: (13.4 mg, 0.022 mmol, 5% yield, 99% purity, 99% ee, OR: +60.1). ¹H NMR (CDCl₃, 400 MHz) δ 7.81-7.89 (m, 2H), 7.51 (br d, J=5.0 Hz, 2H), 7.37-7.44 (m, 2H), 7.27-7.36 (m, 5H), 7.12 (br s, 2H), 7.05 (br dd, J=2.7, 1.1 Hz, 1H), 4.75 (br d, J=2.1 Hz, 2H), 4.61-4.71 (m, 1H), 4.49-4.59 (m, 1H), 4.05-4.17 (m, 1H), 3.71 (br d, J=4.3 Hz, 2H), 3.12-3.24 (m, 2H), 2.00 (br s, 2H), 1.83-1.94 (m, 2H) ppm. LC-MS (ESI+): 608.0 (M+H+); HPLC RT: 3.867 min (Method: VILLA).

### Synthesis of (S,E)-N′-((4-chlorophenyl)sulfonyl)-3-(4-fluorophenyl)-4-phenyl-N-(4-sulfamoylbutyl)-4,5-dihydro-1H-pyrazole-1-carboximidamide (Compound 101) & (R,E)-N′-((4-chlorophenyl)sulfonyl)-3-(4-fluorophenyl)-4-phenyl-N-(4-sulfamoylbutyl)-4,5-dihydro-1H-pyrazole-1-carboximidamide (Compound 102)

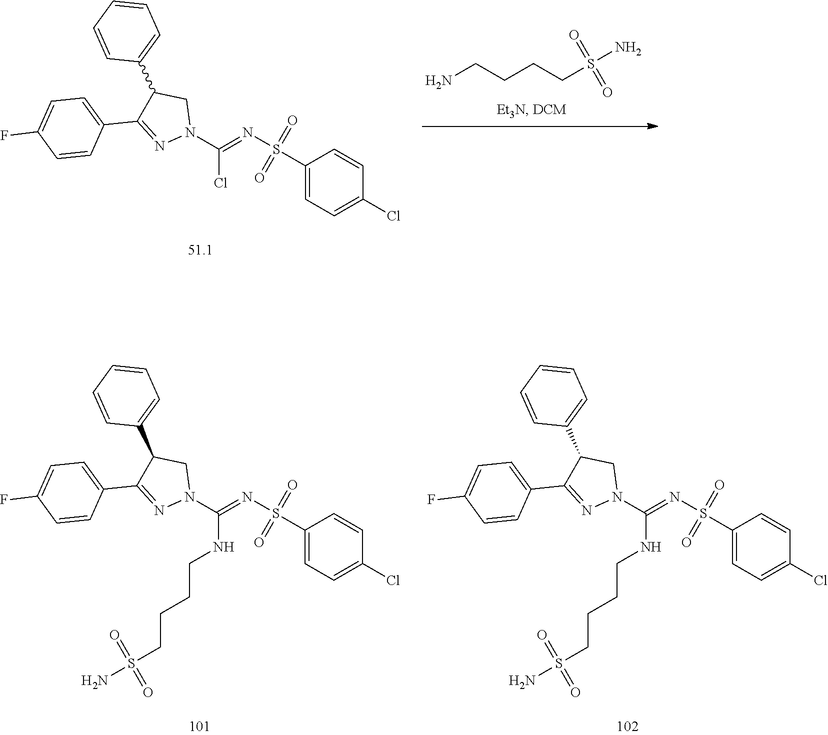 [SMILES: O=S(=O)(/N=C(\Cl)N1CC(c2ccccc2)C(c2ccc(F)cc2)=N1)c1ccc(Cl)cc1 ; NCCCCS(N)(=O)=O ; NS(=O)(=O)CCCCN/C(=N\S(=O)(=O)c1ccc(Cl)cc1)N1C[C@H](c2ccccc2)C(c2ccc(F)cc2)=N1 ; NS(=O)(=O)CCCCN/C(=N\S(=O)(=O)c1ccc(Cl)cc1)N1C[C@@H](c2ccccc2)C(c2ccc(F)cc2)=N1]

### Synthesis of Compounds 101 and 102

To a stirred solution of 4-aminobutane-1-sulfonamide (88 mg, 0.58 mmol, 1.1 eq.) and triethylamine (0.22 mL, 1.6 mmol, 3.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (250 mg, 0.52 mmol, 51.1). After stirring at rt for 18 h, the reaction mixture was diluted with dichloromethane (20 mL) and washed with ammonium chloride (20 mL) and water (20 mL). The organic extract was dried over magnesium sulfate, filtered, and concentrated in vacuo. The residue was purified by flash chromatography on silica eluting with ethyl acetate/heptane mixtures (0/100 to 70/30, v/v), affording a colorless oil which was further subjected to SFC separation conditions to resolve the two enantiomers as white solids: Compound 101 (25.2 mg, 0.04 mmol, 8% yield, 95% purity, 99% ee, OR: −103.5) and Compound 102 (38.4 mg, 0.06 mmol, 12% yield, 93% purity, 99% ee, OR: +89.9).

Method of the SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 101: ¹H NMR (DMSO-d₆, 400 MHz) δ 8.12 (br s, 1H), 7.79 (dt, J=8.7, 2.5 Hz, 4H), 7.54 (d, J=8.6 Hz, 2H), 7.30-7.38 (m, 2H), 7.19-7.28 (m, 5H), 6.76 (s, 2H), 5.06 (dd, J=11.1, 4.3 Hz, 1H), 4.50 (t, J=11.2 Hz, 1H), 4.03 (br dd, J=11.1, 4.3 Hz, 1H), 3.18 (br s, 2H), 2.90-3.00 (m, 2H), 1.54-1.74 (m, 4H) ppm. LC-MS (ESI+): 592.1 (M+H+).

Compound 102: ¹H NMR (DMSO-d₆, 400 MHz) δ 8.12 (br s, 1H), 7.75-7.84 (m, 4H), 7.54 (d, J=8.5 Hz, 2H), 7.29-7.36 (m, 2H), 7.19-7.27 (m, 5H), 6.76 (s, 2H), 5.06 (dd, J=11.1, 4.2 Hz, 1H), 4.50 (t, J=11.2 Hz, 1H), 3.99-4.05 (m, 1H), 3.28-3.31 (m, 2H), 2.95 (br t, J=7.3 Hz, 2H), 1.54-1.74 (m, 4H) ppm. LC-MS (ESI+): 592.1 (M+H+).

### Synthesis of (S,Z)-3-(4-chlorophenyl)-N′-((4-chlorophenyl)sulfonyl)-4-isopropyl-N-(2-sulfamoylethyl)-4,5-dihydro-1H-pyrazole-1-carboximidamide (Compound 103) & (R,Z)-3-(4-chlorophenyl)-N′-((4-chlorophenyl)sulfonyl)-4-isopropyl-N-(2-sulfamoylethyl)-4,5-dihydro-1H-pyrazole-1-carboximidamide (Compound 104)

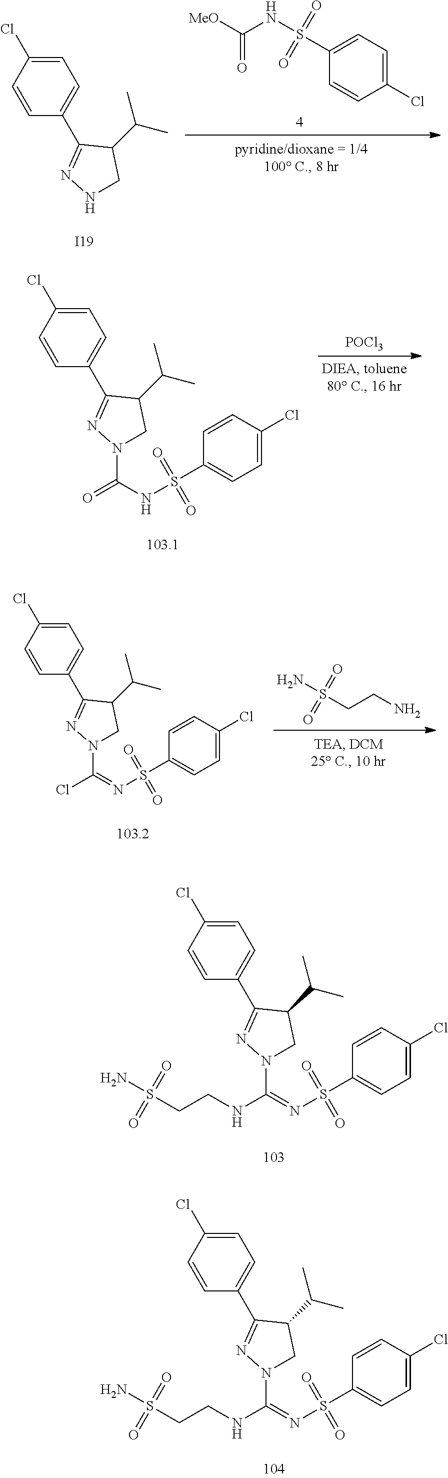

### Synthesis of 103.1

To a solution of 119 (370 mg, 1.66 mmol, 1 eq) in dioxane (4 mL) and pyridine (1 mL) at rt was added methyl ((4-chlorophenyl)sulfonyl)carbamate (435.53 mg, 1.74 mmol, 1.05 eq). After stirring at 100° C. for 8 h, the reaction mixture was concentrated under reduced pressure to give a residue, which was triturated with methyl tertiary butyl ether (5 mL) to give 103.1 (150 mg, 306.58 μmol, 18%) as a white solid. LCMS (ESI+): m/z 223.1 (M+H)⁺. ¹H NMR: 400 MHz, DMSO-d₆ δ ppm 0.51 (d, J=6.85 Hz, 3H), 0.91 (d, J=6.85 Hz, 3H), 1.97-2.09 (m, 1H), 3.71-3.86 (m, 3H), 7.54 (d, J=8.56 Hz, 2H), 7.71 (d, J=8.56 Hz, 2H), 7.95 (dd, J=13.75, 8.62 Hz, 4H), 11.34 (br s, 1H).

### Synthesis of 103.2

To a solution of 103.1 (150 mg, 340.64 μmol, 1 eq) in toluene (2 mL) at rt were added diisopropylethylamine (77.13 μL, 442.84 μmol, 1.3 eq) and phosphorous oxychloride (37.99 μL, 408.77 μmol, 1.2 eq). After stirring at 80° C. for 6 h, the reaction mixture was quenched by water (10 mL) and extracted with ethyl acetate (5 mL×2). The combined organic layer was washed with brine (5 mL×2), dried over Na₂SO₄, filtered, and concentrated under reduced pressure. The residue was triturated with ethanol (5 mL) to give 103.2 (120 mg, 235.40 μmol, 69%) as a white solid. LCMS (ESI+): m/z 459.9 (M+H)⁺. ¹H NMR: 400 MHz, DMSO-d₆ δ ppm 0.52-0.60 (m, 3H), 0.97 (d, J=6.79 Hz, 3H), 2.08 (td, J=6.94, 3.16 Hz, 1H), 3.97-4.22 (m, 3H), 7.59 (d, J=8.58 Hz, 2H), 7.64-7.71 (m, 2H), 7.84 (d, J=8.58 Hz, 2H), 7.90-7.96 (m, 2H)

### Synthesis of Compounds 103 and 104

To a solution of 103.1 (120 mg, 261.56 μmol, 1 eq) in dichloromethane (2 mL) at rt were added 2-aminoethane-1-sulfonamide (50.41 mg, 313.87 μmol, 1.2 eq, HCl salt) and triethylamine (66.17 mg, 653.89 μmol, 2.5 eq). After stirring at rt for 10 h, the reaction mixture was diluted with water (10 mL) and extracted with ethyl acetate (5 mL×2). The combined organic layer was washed with brine (5 mL×2), dried over Na₂SO₄, filtered, and concentrated under reduced pressure. The residue was purified by silica gel column using petroleum ether and ethyl acetate (5/1, v/v) to obtain racemate (110 mg, 191.19 μmol, 77%) as a white solid, which was further separated by SFC to obtain Compound 104 (16 mg, 27.81 μmol, 11%) as a white solid and Compound 103 (18 mg, 32.94 μmol, 12%) as a white solid. LCMS (ESI+): m/z 546.0 (M+H)⁺.

Method of the SFC: Column: DAICEL CHIRALPAK AD (250 mm*30 mm, 10 μm); Mobile phase A: CO₂ Mobile phase B: [0.1% NH₃H2O in IPA]; Mobile phase B %: 50%; Run time: 12 min.

Compound 103: ¹H NMR: 400 MHz, DMSO-d₆ δ ppm 0.55 (d, J=6.79 Hz, 3H), 0.91 (d, J=6.79 Hz, 3), 2.01 (ddd, J=9.83, 6.62, 3.34 Hz, 1H), 3.20 (br t, J=6.85 Hz, 2H), 3.66 (br d, J=3.46 Hz, 2H), 3.86-3.93 (m, 1H), 3.95-4.04 (m, 1H), 4.15 (br dd, J=10.91, 2.92 Hz, 1H), 7.00 (s, 2H), 7.57 (dd, J=8.40, 5.54 Hz, 4H), 7.87 (dd, J=8.58, 2.27 Hz, 4H), 8.02 (br s, 1H); LCMS (ESI+): m/z 546.0 (M+H)⁺. OR: −1.60.

Compound 104: ¹H NMR: 400 MHz, DMSO-d₆ δ ppm 0.55 (d, J=6.79 Hz, 3H), 0.91 (d, J=6.79 Hz, 3H), 1.96-2.05 (m, 2H), 3.20 (t, J=6.97 Hz, 2H), 3.60-3.71 (m, 2H), 3.86-3.94 (m, 1H), 3.95-4.03 (m, 1H), 4.15 (dd, J=11.15, 3.28 Hz, 1H), 7.00 (s, 2H), 7.52-7.62 (m, 4H), 7.82-7.92 (m, 4H), 8.02 (br t, J=5.54 Hz, 1H); LCMS (ESI+): m/z 546.0 (M+H)⁺. OR: 1.80.

### Example 105 & 106: Synthesis of (S,Z)-3-(4-chlorophenyl)-4-isopropyl-N-(2-sulfamoylethyl)-N′-((4-(trifluoromethyl)phenyl)sulfonyl)-4,5-dihydro-1H-pyrazole-1-carboximidamide (105) & (S,Z)-3-(4-chlorophenyl)-4-isopropyl-N-(2-sulfamoylethyl)-N′-((4-(trifluoromethyl)phenyl)sulfonyl)-4,5-dihydro-1H-pyrazole-1-carboximidamide (106)

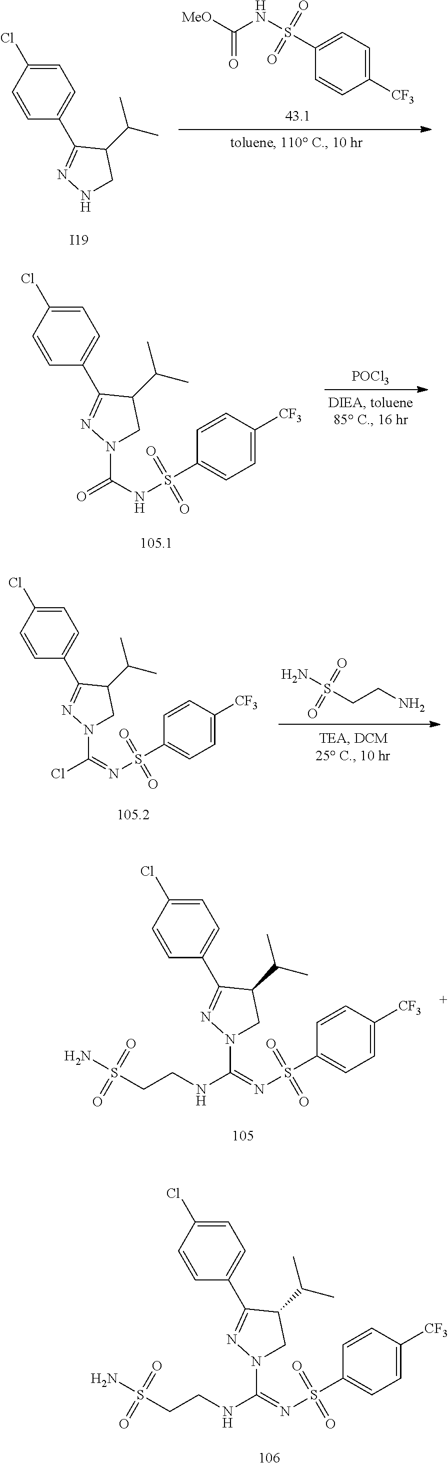

### Synthesis of 105.1

To a solution of 119 (414 mg, 1.86 mmol) in toluene (5 mL) at rt was added 43.1 (579.13 mg, 2.04 mmol, 1.1 eq). After stirring at 110° C. for 10 h, the reaction mixture was concentrated under reduced pressure. The residue was triturated with methyl tertiary butyl ether (5 mL) to give 105.1 (270 mg, 512.77 μmol, 27%) as a white solid. ¹H NMR: 400 MHz CDCl₃ δ ppm 0.64 (d, J=6.91 Hz, 3H), 0.98 (d, J=6.79 Hz, 3H), 2.14 (dtt, J=10.26, 6.81, 6.81, 3.44, 3.44 Hz, 1H), 3.63-3.72 (m, 1H), 3.81 (t, J=11.44 Hz, 1H), 3.88-3.95 (m, 1H), 7.40-7.47 (m, 2H), 7.58-7.67 (m, 2H), 7.82 (d, J=8.34 Hz, 2H), 8.29 (d, J=8.23 Hz, 2H), 8.66 (s, 1H).

### Synthesis of 105.2

To a solution of 105.1 (270 mg, 0.57 mmol, 1 eq) in toluene (5 mL) at rt was added N, N-diisopropylethylamine (0.15 mL, 0.85 mmol, 1.5 eq). After stirring at rt for 1 hr. Phosphorus oxychloride (0.11 g, 0.74 mmol, 0.07 mL, 1.3 eq) was added at rt. After stirring at 85° C. for 16 h, the reaction mixture was cooled to rt and concentrated under reduced pressure to give 105.2 (610 mg, 557.54 μmol, 98%) as a yellow solid. LCMS (ESI+): m/z 491.9 (M+H)⁺.

### Synthesis of Compounds 105 and 106

To a solution of 105.2 (610 mg, 1.24 mmol, 1 eq) in dichloromethane (6 mL) at rt were added triethylamine (626.85 mg, 6.19 mmol, 0.86 mL, 5 eq) and 2-aminoethane-1-sulfonamide (230.75 mg, 1.86 mmol, 1.5 eq). After stirring at rt for 10 h, the reaction mixture was concentrated under reduced pressure. The residue was purified by silica gel column using petroleum ether and ethyl acetate (5/1, v/v) to give a racemate, which was separated by SFC to obtained Compound 105 (76.70 mg, 128.26 μmol, 10%) as a white solid and Compound 106 (73.50 mg, 126.71 μmol, 10%) as a white solid.

Method of SFC: Column: DAICEL CHIRALCEL OD (250 mm×30 mm, 10 μm); Mobile phase A: CO₂; Mobile phase B: [0.1% NH₃-H₂O IPA]; B %: 37%-37%, Run time: 10 min.

Compound 105: ¹H NMR: 400 MHz DMSO-d₆ δ ppm 8.06 (d, J=8.11 Hz, 2H), 7.82-7.93 (m, 4H), 7.56 (d, J=8.58 Hz, 2H), 6.76-7.24 (m, 2H), 4.05-4.14 (m, 1H), 3.99 (t, J=10.91 Hz, 1H), 3.85-3.93 (m, 1H), 3.58-3.75 (m, 2H), 3.21 (t, J=7.03 Hz, 2H), 2.01 (dq, J=9.95, 6.74 Hz, 1H), 0.89 (d, J=6.79 Hz, 3H), 0.53 (d, J=6.79 Hz, 3H). LCMS (ESI+): m/z 580.0 (M+H)⁺. OR: −1.90.

Compound 106: ¹H NMR: 400 MHz DMSO-d₆ δ ppm 8.07 (d, J=8.13 Hz, 2H), 7.82-7.94 (m, 4H), 7.57 (d, J=8.63 Hz, 2H), 7.00 (br s, 2H), 4.06-4.16 (m, 1H), 4.00 (t, J=10.94 Hz, 1H), 3.87-3.94 (m, 1H), 3.67 (br s, 2H), 3.22 (t, J=7.00 Hz, 2H), 1.95-2.07 (m, 1H), 0.90 (d, J=6.88 Hz, 3H), 0.54 (d, J=6.75 Hz, 3H). LCMS (ESI+): m/z 580.0 (M+H)⁺. OR: 1.80.

### Synthesis of (S,Z)-N′-((4-chlorophenyl)sulfonyl)-3-(4-fluorophenyl)-N-methyl-4-phenyl-N-(2-sulfamoylethyl)-4,5-dihydro-1H-pyrazole-1-carboximidamide (Compound 107) & (R,Z)-N′-((4-chlorophenyl)sulfonyl)-3-(4-fluorophenyl)-N-methyl-4-phenyl-N-(2-sulfamoylethyl)-4,5-dihydro-1H-pyrazole-1-carboximidamide (Compound 108)

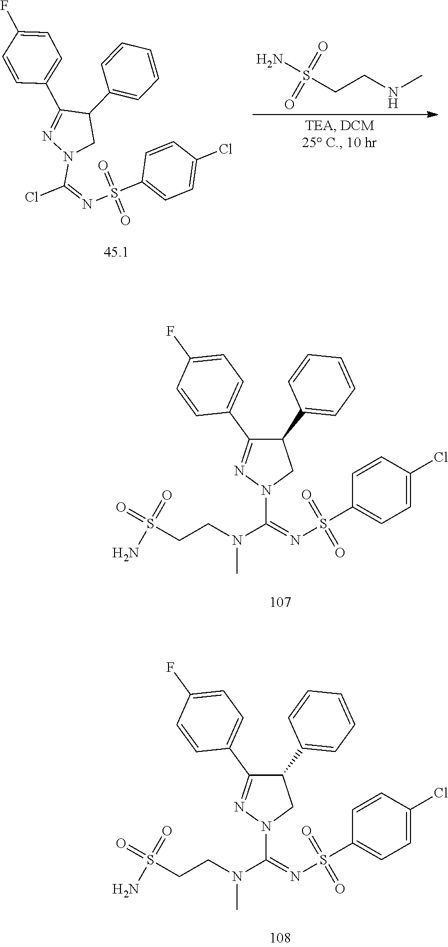

### Synthesis of Compounds 107 and 108

To a solution of 2-(methylamino)ethane-1-sulfonamide (300 mg, 1.72 mmol, 1 eq) in dichloromethane (20 mL) at rt were added triethylamine (1.2 mL, 8.59 mmol, 5 eq) and 45.1 (1.23 g, 2.58 mmol, 1.2 eq). After stirring at rt for 12 h, the mixture was concentrated under reduced pressure. The residue was purified by column on silica gel using ethyl acetate to give racemate (400 mg, 733 μmol, 42%) as a white solid. LCMS (ESI+): m/z 578.3 (M+H)⁺.

The racemate (400 mg, 733 μmol, 1 eq) was separated by the SFC to give Compound 107 (103 mg, 183.25 μmol, 11%), and Compound 108 (80.9 mg, 138.39 μmol, 8%) as a white solid.

Method of the SFC: Column daicel chiralcel 01 (250 mm*30 mm, 10 μm); Mobile phase A: CO2; Mobile phase B: 0.1% NH₃H₂O MeOH; B %: 50%, Run time: 15 min.

Compound 107: ¹H NMR: 400 MHz, DMSO-d₆ δ ppm 2.07 (s, 1H) 3.10 (s, 3H) 3.36-3.51 (m, 2H) 3.72-3.92 (m, 3H) 4.56 (t, J=11.15 Hz, 1H) 5.03 (dd, J=11.27, 5.07 Hz, 1H) 6.99 (s, 2H) 7.15-7.27 (m, 3H) 7.28-7.35 (m, 4H) 7.52 (d, J=8.58 Hz, 2H) 7.66 (dd, J=8.70, 5.60 Hz, 2H) 7.80 (d, J=8.58 Hz, 2H); LCMS (ESI+): m/z 578.2 (M+H)⁺. OR: −122.60.

Compound 108: ¹H NMR: 400 MHz, DMSO-d₆ δ ppm 2.07 (s, 1H) 3.10 (s, 3H) 3.35-3.51 (m, 2H) 3.73-3.91 (m, 3H) 4.56 (t, J=11.09 Hz, 1H) 5.03 (dd, J=11.32, 5.13 Hz, 1H) 6.98 (s, 2H) 7.15-7.27 (m, 3H) 7.28-7.35 (m, 4H) 7.49-7.55 (m, 2H) 7.66 (dd, J=8.82, 5.48 Hz, 2H) 7.77-7.83 (m, 2H); LCMS (ESI+): m/z 578.2 (M+H)⁺; OR: 114.40.

### Synthesis of (S,Z)-3-(4-chlorophenyl)-N′-((4-chlorophenyl)sulfonyl)-N-methyl-4-phenyl-N-(2-sulfamoylethyl)-4,5-dihydro-1H-pyrazole-1-carboximidamide (Compound 109) & (S,Z)-3-(4-chlorophenyl)-N′-((4-chlorophenyl)sulfonyl)-N-methyl-4-phenyl-N-(2-sulfamoylethyl)-4,5-dihydro-1H-pyrazole-1-carboximidamide (Compound 110)

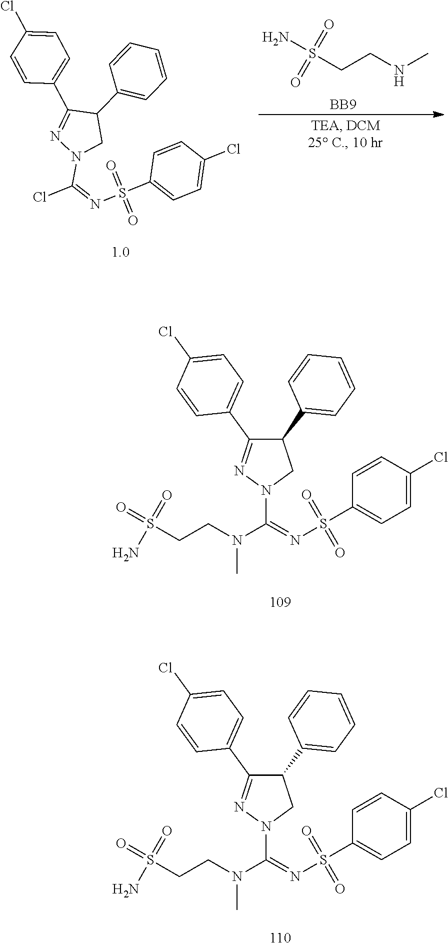

### Synthesis of Compounds 109 and 110

To a solution of 1.0 (1.27 g, 2.58 mmol, 1.5 eq) in dichloromethane (10 mL) at rt were added triethylamine (869.08 g, 8.59 mmol, 1.20 mL, 5 eq) and 2-(methylamino)ethane-1-sulfonamide (300 mg, 1.72 mmol, 1 eq). After stirring at rt for 12 h, the mixture was concentrated under reduced pressure. The residue was purified by column on silica gel using petroleum ether and ethyl acetate (1/1, v/v) to give racemate (900 mg, 908.28 μmol, 60%) as a white solid. LCMS (ESI+): m/z 593.07.0 (M+H)⁺.

The racemate (200 mg, 336.40 μmol, 1 eq) was separated by SFC to give Compound 109 (100 mg, 168.82 μmol, 96%) as a white solid and Compound 110 (100 mg, 158.61 μmol, 94%) as a white solid.

Method of SFC: Column: Chiralpak AD-3, 50×4.6 mm L.D, 3 μm); Mobile phase A: CO2; Mobile phase B: isopropanol (0.1% isopropanol m, v/v); B %: 50%, Run time: 15 min.

Compound 109: ¹H NMR (400 MHz, DMSO-d₆) δ ppm 3.10 (s, 3H), 3.42 (td, J=8.85, 5.94 Hz, 2H), 3.72-3.89 (m, 3H), 4.57 (t, J=11.19 Hz, 1H), 5.04 (dd, J=11.32, 5.19 Hz, 1H), 6.98 (s, 2H), 7.21-7.27 (m, 1H), 7.28-7.35 (m, 4H), 7.41 (d, J=8.76 Hz, 2H), 7.50-7.55 (m, 2H), 7.61 (d, J=8.63 Hz, 2H), 7.77-7.84 (m, 2H); LCMS (ESI+): m/z 594.0 (M+H)⁺; OR: −98.70.

Compound 110: ¹H NMR (400 MHz, DMSO-d₆) δ ppm 3.10 (s, 3H), 3.38-3.49 (m, 2H), 3.74-3.89 (m, 3H), 4.57 (br t, J=11.21 Hz, 1H), 5.03 (br dd, J=11.32, 5.01 Hz, 1H), 6.98 (s, 2H), 7.25 (br d, J=6.32 Hz, 1H), 7.28-7.35 (m, 4H), 7.41 (br d, J=8.46 Hz, 2H), 7.53 (br d, J=8.46 Hz, 2H), 7.61 (br d, J=8.46 Hz, 2H), 7.80 (br d, J=8.46 Hz, 2H); LCMS (ESI+): m/z 594.0 (M+H)⁺; OR: 94.60.

### Synthesis of (S,Z)-1-((((4-chlorophenyl)sulfonyl)imino)(3-(4-fluorophenyl)-4-phenyl-4,5-dihydro-1H-pyrazol-1-yl)methyl)piperidine-4-sulfonamide (Compound 111) & (R,Z)-1-((((4-chlorophenyl)sulfonyl)imino)(3-(4-fluorophenyl)-4-phenyl-4,5-dihydro-1H-pyrazol-1-yl)methyl)piperidine-4-sulfonamide (Compound 112)

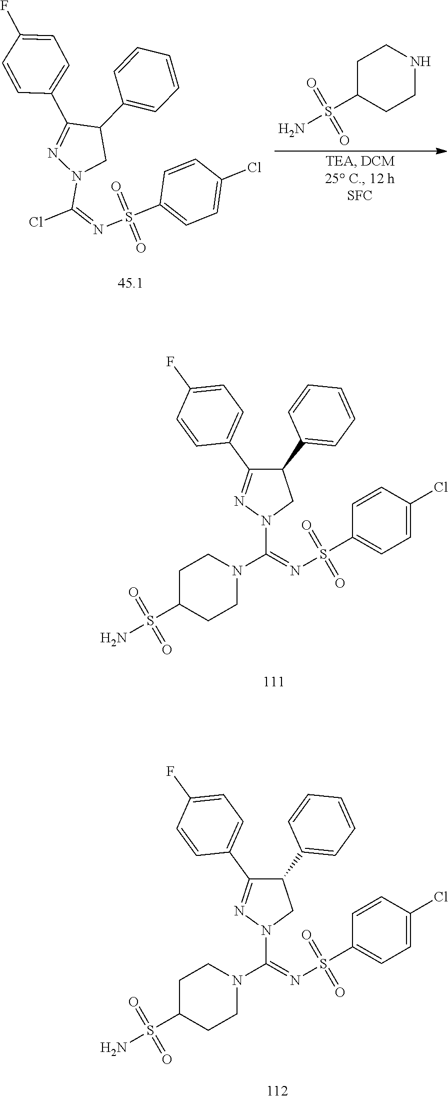

### Synthesis of Compounds 111 and 112

To a solution of 45.1 (760 mg, 1.60 mmol, 1 eq) in dichloromethane (50 mL) at rt were added triethylamine (1.11 mL, 7.98 mmol, 5 eq) and piperidine-4-sulfonamide (262.02 mg, 1.60 mmol, 1 eq). After stirring at rt for 12 h, the mixture was concentrated under reduced pressure. The residue was purified by column on silica gel using petroleum ether and ethyl acetate (1/1, v/v) to yield racemic mixture, which was separated by SFC to give Compound 111 (36.7 mg, 6.08 mmol, 4%) as white solid and Compound 112 (32.7 mg, 5.42 mmol, 3%) as a white solid.

Method of SFC: Column: DAICEL CHIRALCEL OD (250 mm×30 mm, 10 μm); A: CO2, Mobile phase B: [Neu-isopropanol]; Ratio: 50%, run time: 12 min.

Compound 111: ¹H NMR: 400 MHz DMSO-d₆ δ ppm 1.77 (qd, J=12.38, 3.87 Hz, 1H), 1.89-2.03 (m, 1H), 2.14 (br s, 2H), 3.17-3.31 (m, 3H), 3.63 (dd, J=11.27, 4.71 Hz, 1H), 4.12 (br t, J=14.25 Hz, 2H), 4.42 (t, J=11.27 Hz, 1H), 4.96 (dd, J=11.21, 4.65 Hz, 1H), 6.90 (s, 2H), 7.17-7.27 (m, 5H), 7.28-7.35 (m, 2H), 7.51 (d, J=8.58 Hz, 2H), 7.69 (dd, J=8.82, 5.48 Hz, 2H), 7.77 (d, J=8.58 Hz, 2H); LCMS (ESI+): m/z 604.0 (M+1)⁺ OR: −89.68.

Compound 112: ¹H NMR: 400 MHz DMSO-d₆ δ ppm 1.77 (qd, J=12.36, 3.70 Hz, 1H) 1.96 (qd, J=12.26, 3.99 Hz, 1H) 2.14 (br s, 2H) 3.17-3.30 (m, 3H) 3.63 (dd, J=11.21, 4.65 Hz, 1H) 4.12 (br t, J=14.25 Hz, 2H) 4.42 (t, J=11.21 Hz, 1H) 4.96 (dd, J=11.09, 4.65 Hz, 1H) 6.90 (s, 2H) 7.17-7.27 (m, 5H) 7.31 (d, J=7.03 Hz, 2H) 7.51 (d, J=8.58 Hz, 2H) 7.69 (dd, J=8.70, 5.48 Hz, 2H) 7.77 (d, J=8.46 Hz, 2H) LCMS (ESI+): m/z 604.0 (M+1)⁺; OR: 87.67.

### Synthesis of (S,Z)-1-((3-(4-chlorophenyl)-4-phenyl-4,5-dihydro-1H-pyrazol-1-yl)(((4-chlorophenyl)sulfonyl)imino)methyl)piperidine-4-sulfonamide (Compound 113) & (S,Z)-1-((3-(4-chlorophenyl)-4-phenyl-4,5-dihydro-1H-pyrazol-1-yl)(((4-chlorophenyl)sulfonyl)imino)methyl)piperidine-4-sulfonamide (Compound 114)

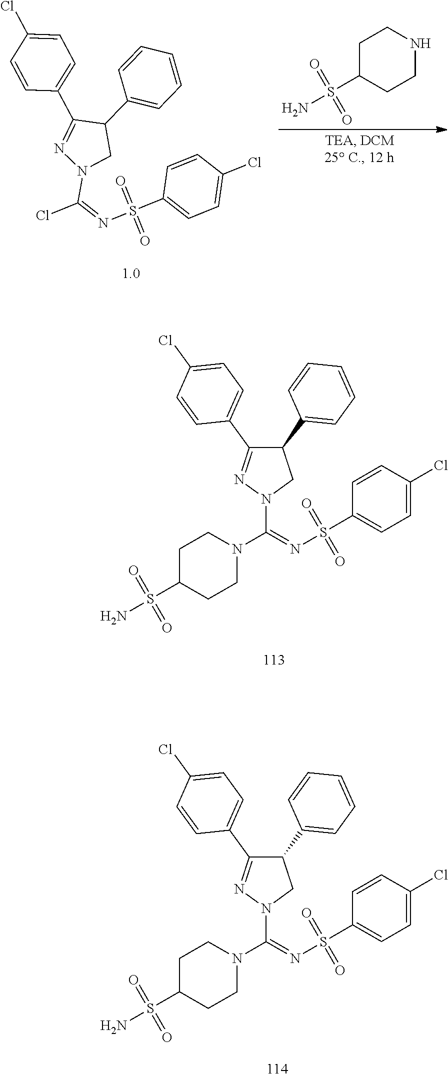

### Synthesis of Compounds 113 and 114

To a solution of 1.0 (500 mg, 1.01 mmol, 1 eq) in dichloromethane (5 mL) at rt were added triethylamine (351.45 μL, 2.53 mmol, 2.5 eq) and piperidine-4-sulfonamide (202.69 mg, 1.01 mmol, 1 eq, HCl). After stirring at rt for 12 h, the mixture was concentrated under reduced pressure. The residue, which was purified by column on silica gel using petroleum ether and ethyl acetate (1/1, v/v) to obtain the racemate (470 mg, 0.76 mmol, 75%) as a white solid. LCMS (ESI+): m/z 620.1 (M+1)⁺. The racemate (300 mg, 483 μmol, 1 eq) was separated by SFC to obtained Compound 113 (92.8 mg, 150 μmol, 31%) as a white solid and Compound 114 (94.6 mg, 153 μmol, 32%) as a white solid.

Method of SFC: Column: DAICEL CHIRALCEL OD (250 mm×50 mm, 10 μm); Mobile phase: [Neu-isopropanol]; B %: 50%-50%, 10 min.

Compound 113: ¹H NMR: 400 MHz DMSO-d6 δ 1.70-1.88 (m, 1H), 1.89-2.05 (m, 1H), 2.14 (br d, J=1.59 Hz, 2H), 3.30 (br s, 3H), 3.65 (dd, J=11.19, 4.83 Hz, 1H), 4.12 (br t, J=14.06 Hz, 2H), 4.42-4.50 (m, 1H), 4.97 (dd, J=11.31, 4.83 Hz, 1H), 6.91 (s, 2H), 7.21-7.29 (m, 3H), 7.29-7.36 (m, 2H), 7.44 (d, J=8.68 Hz, 2H), 7.52 (d, J=8.56 Hz, 2H), 7.64 (d, J=8.68 Hz, 2H), 7.78 (d, J=8.56 Hz, 2H); LCMS (ESI+): m/z 620.0 (M+1)⁺ OR: −64.82.

Compound 114: ¹H NMR: 400 MHz DMSO-d₆ δ 1.69-1.86 (m, 1H), 1.89-2.04 (m, 1H), 2.07-2.22 (m, 2H), 3.19-3.32 (m, 3H), 3.65 (dd, J=11.25, 4.89 Hz, 1H), 4.12 (br t, J=14.00 Hz, 2H), 4.44 (t, J=11.31 Hz, 1H), 4.97 (dd, J=11.25, 4.89 Hz, 1H), 6.91 (s, 2H), 7.22-7.28 (m, 3H), 7.29-7.35 (m, 2H), 7.44 (d, J=8.68 Hz, 2H), 7.52 (d, J=8.56 Hz, 2H), 7.64 (d, J=8.56 Hz, 2H), 7.78 (d, J=8.56 Hz, 2H); LCMS (ESI+): m/z 620.0 (M+1)⁺; OR: 74.73.

### Synthesis of (S,Z)-3-(4-chlorophenyl)-N′-((4-chlorophenyl)sulfonyl)-4-phenyl-N-((1r,3S)-3-sulfamoylcyclobutyl)-4,5-dihydro-1H-pyrazole-1-carboximidamide (Compound 115), (S,Z)-3-(4-chlorophenyl)-N′-((4-chlorophenyl)sulfonyl)-4-phenyl-N-((1s,3R)-3-sulfamoylcyclobutyl)-4,5-dihydro-1H-pyrazole-1-carboximidamide, (R,Z)-3-(4-chlorophenyl)-N′-((4-chlorophenyl)sulfonyl)-4-phenyl-N-((1 r,3R)-3-sulfamoylcyclobutyl)-4,5-dihydro-1H-pyrazole-1-carboximidamide (117) & (R,Z)-3-(4-chlorophenyl)-N′-((4-chlorophenyl)sulfonyl)-4-phenyl-N-((1s,3S)-3-sulfamoylcyclobutyl)-4,5-dihydro-1H-pyrazole-1-carboximidamide (Compound 118)

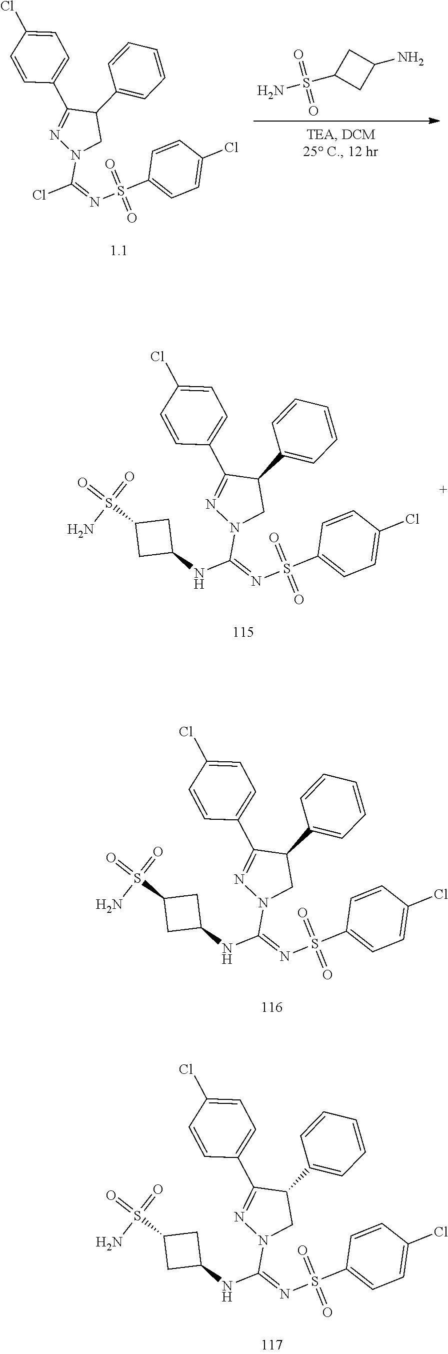

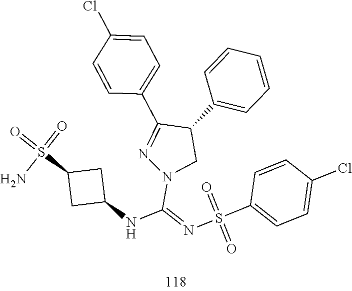 [SMILES: NS(=O)(=O)[C@H]1C[C@@H](N/C(=N/S(=O)(=O)c2ccc(Cl)cc2)N2C[C@@H](c3ccccc3)C(c3ccc(Cl)cc3)=N2)C1]

To a solution of 1.0 (1 g, 3.15 mmol, 1 eq) in dichloromethane (5 mL) at rt were added triethylamine (706.10 μL, 5.07 mmol, 2.5 eq) and 3-aminocyclobutane-1-sulfonamide (378.77 mg, 2.03 mmol, 1 eq). After stirring at rt for 12 h, the mixture was concentrated under reduced pressure. The residue was purified by column on silica gel using petroleum ether and ethyl acetate (1/1, v/v) to give mixture of diastereomers (860 mg, 1.35 mmol, 66%) as a white solid. LCMS (ESI+): m/z 606.1.0 (M+H)⁺.

Method of the SFC: The diastereomers (800 mg, 1.32 mmol, 1 eq) were separated by the first SFC to give Compound 115 (102 mg, 161.78 μmol, 12%) as a white solid, Compound 116 (32 mg, 52.76 μmol, 4%) as a white solid and a racemic mixture of Compound 117 and Compound 118. The racemic mixture of Compound 117 and Compound 118 was further separated by the second SFC to obtain Compound 117 (135 mg, 222.57 μmol, 16%) as a white solid and Compound 118 (78 mg, 127.44 μmol, 9%) as a white solid. Absolute stereochemistry was not determined. Diastereomers with (-) OR were arbitrary assigned as S isomer.

Method of the first SFC: Column: REGIS (s,s) WHELK-01 (250 mm×30 mm, 10 μm); Mobile phase A: [NH₃H₂O (0.1%) in ethanol]; Mobile phase B: CO₂; B %: 60%, Run time: 10 min.

Method of the second SFC: Column: DAICEL CHIRALPAK AD (250 mm×30 mm, 10 μm); Mobile phase A: [NH₃H₂O (0.1%) in methanol]; Mobile phase B: CO₂; B %: 65%, Run time: 20 min.

Compound 115: ¹H NMR: 400 MHz, DMSO-d₆ δ ppm 2.07 (s, 1H), 2.30-2.46 (m, 3H), 2.54 (s, 1H), 3.46 (quin, J=8.44 Hz, 1H), 3.94 (dd, J=11.25, 4.28 Hz, 1H), 4.14-4.32 (m, 1H), 4.48 (t, J=11.31 Hz, 1H), 5.05 (dd, J=11.19, 4.34 Hz, 1H), 6.80 (s, 2H), 7.18-7.35 (m, 5H), 7.45 (d, J=8.68 Hz, 2H), 7.50-7.60 (m, 2H), 7.71-7.82 (m, 4H), 8.13 (br d, J=5.50 Hz, 1H); LCMS (ESI+): m/z 606.0 (M+H)⁺; OR: −119.57.

Compound 116: ¹H NMR: 400 MHz, DMSO-d₆ δ ppm 2.55-2.71 (m, 4H), 3.50-3.67 (m, 1H), 3.96 (br dd, J=11.25, 4.16 Hz, 1H), 4.38-4.69 (m, 2H), 5.06 (dd, J=11.19, 4.34 Hz, 1H), 6.86 (s, 2H), 7.17-7.29 (m, 3H), 7.29-7.38 (m, 2H), 7.46 (d, J=8.68 Hz, 2H), 7.53 (d, J=8.56 Hz, 2H), 7.77 (dd, J=16.26, 8.56 Hz, 4H), 8.19 (br d, J=5.99 Hz, 1H); LCMS (ESI+): m/z 606.0 (M+H)⁺; OR: −105.34.

Compound 117: ¹H NMR:, 400 MHz, DMSO-d₆ δ ppm 2.29-2.48 (m, 4H), 3.46 (m, J=8.34 Hz, 1H), 3.94 (br dd, J=11.19, 4.10 Hz, 1H), 4.24 (br s, 1H), 4.48 (br t, J=11.25 Hz, 1H), 5.05 (br dd, J=11.19, 4.22 Hz, 1H), 6.80 (br s, 2H), 7.17-7.37 (m, 5H), 7.45 (d, J=8.56 Hz, 2H), 7.55 (d, J=8.56 Hz, 2H), 7.69-7.84 (m, 4H), 8.13 (br s, 1H); LCMS (ESI+): m/z 606.0 (M+H)⁺; OR: 116.16.

Compound 118: ¹H NMR: 400 MHz, DMSO-d₆ δ ppm 2.07 (s, 1H), 2.53-2.70 (m, 4H), 3.51-3.65 (m, 1H), 3.96 (br dd, J=11.19, 4.22 Hz, 1H), 4.40-4.66 (m, 2H), 5.05 (dd, J=11.19, 4.34 Hz, 1H), 6.86 (s, 2H), 7.17-7.28 (m, 3H), 7.28-7.37 (m, 2H), 7.45 (d, J=8.56 Hz, 2H), 7.53 (d, J=8.56 Hz, 2H), 7.76 (dd, J=16.38, 8.56 Hz, 4H), 8.19 (br d, J=5.75 Hz, 1H); LCMS (ESI+): m/z 606.0 (M+1)⁺; OR: 105.11.

### Synthesis of (S,Z)-3-(4-chlorophenyl)-N′-((4-chlorophenyl)sulfonyl)-4-phenyl-N-((1s,4R)-4-sulfamoylcyclohexyl)-4,5-dihydro-1H-pyrazole-1-carboximidamide (Compound 119); (S,Z)-3-(4-chlorophenyl)-N′-((4-chlorophenyl)sulfonyl)-4-phenyl-N-((1r,4S)-4-sulfamoylcyclohexyl)-4,5-dihydro-1H-pyrazole-1-carboximidamide (Compound 120), (R,Z)-3-(4-chlorophenyl)-N′-((4-chlorophenyl)sulfonyl)-4-phenyl-N-((1s,4S)-4-sulfamoylcyclohexyl)-4,5-dihydro-1H-pyrazole-1-carboximidamide (Compound 121) & (R,Z)-3-(4-chlorophenyl)-N′-((4-chlorophenyl)sulfonyl)-4-phenyl-N-((1 r,4R)-4-sulfamoylcyclohexyl)-4,5-dihydro-1H-pyrazole-1-carboximidamide (Compound 122)

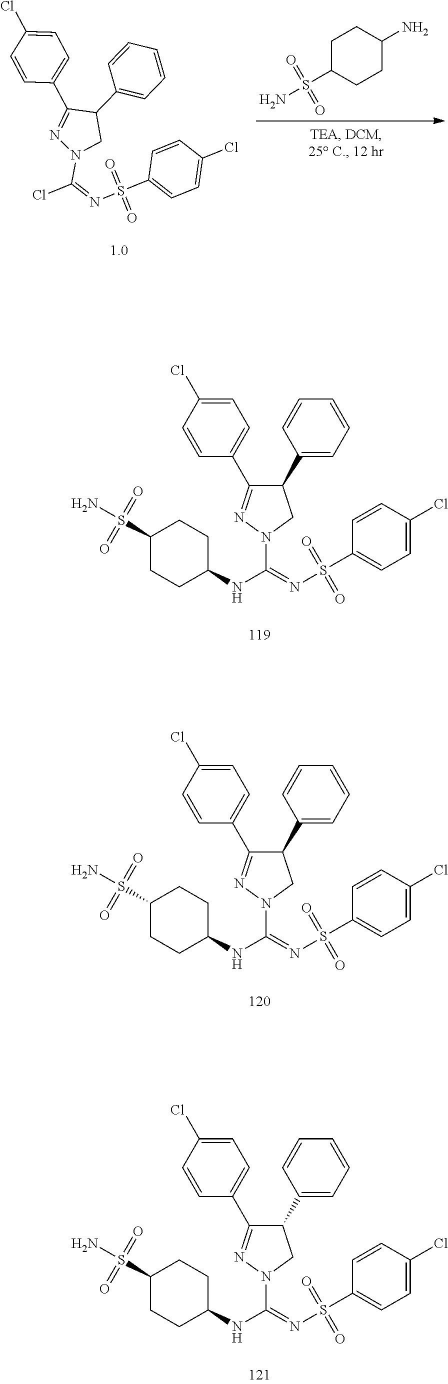

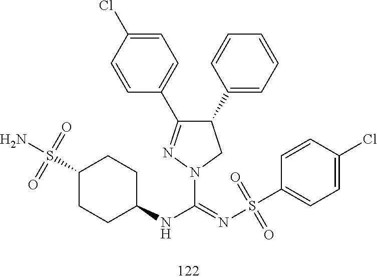 [SMILES: NS(=O)(=O)[C@H]1CC[C@H](N/C(=N/S(=O)(=O)c2ccc(Cl)cc2)N2C[C@@H](c3ccccc3)C(c3ccc(Cl)cc3)=N2)CC1]

### Synthesis of Compounds 119-122

To a solution of 1.0 (2 g, 2.43 mmol, 1 eq) in dichloromethane (20 ml-) at rt were added triethylamine (1.23 g, 12.13 mmol, 1.69 mL, 5 eq) and 4-aminocyclohexane-1-sulfonamide (520.71 mg, 2.43 mmol, 1 eq). After stirring at rt for 12 h, the mixture was concentrated under reduced pressure. The residue was purified by prep-HPLC to give two racemic mixtures as white solids. The two racemic mixtures were separated by SFC to give Compound 119 (104.1 mg, 161.58 μmol, 7%) as a white solid, Compound 121 (127.1 mg, 200.28 μmol, 8%) as a white solid, Compound 120 (36.7 mg, 57.83 μmol, 2.3%) as a white solid and Compound 121 (46.4 mg, 73.12 μmol, 3%) as a white solid. Absolute stereochemistry was not determined. Diastereomers with (-) OR were arbitrary assigned as S isomer.

Method of prep-HPLC: Instrument: ACSTJ-GX-N; Column: Phenomenex luna C18 250×50 mm×10 μm; Mobile phase: A for H₂O=(0.04% HCl) and B for acetonitrile; Gradient: B from 60% to 80% in 10 min; Flow rate: 80 mL/min; Wavelength: 220&254 nm

Method of the SFC separation for Compounds 119 & 121: Column: PHENOMENEX LUNA C18 (250 mm×30 mm, 10 μm); Mobile phase A: [water (HCl) in ACN]; Mobile phase B: CO2; B %: 60%-80%, Run time: 10 min

Method of the SFC separation for Compounds 120 & 122: Column: DAICEL CHIRALPAK AD (250 mm×30 mm, 10 μm); Mobile phase A: [NH₃H₂O (0.1%) in ethanol]; Mobile phase B: CO₂; B %: 50%, Run time: 18 min

Compound 119: ¹H NMR: 400 MHz, DMSO-d₆; δ ppm 1.21-1.60 (m, 4H), 1.93 (brt, J=11.13 Hz, 2H), 2.05-2.18 (m, 2H), 2.75 (br t, J=11.86 Hz, 1H), 3.62-3.82 (m, 1H), 3.90-4.09 (m, 1H), 4.51 (t, J=11.25 Hz, 1H), 5.06 (dd, J=11.07, 4.34 Hz, 1H), 6.72 (s, 2H), 7.19-7.30 (m, 3H), 7.31-7.37 (m, 2H), 7.46 (d, J=8.68 Hz, 2H), 7.52-7.59 (m, 2H), 7.70-7.83 (m, 4H); LCMS (ESI+): m/z 634.0 (M+H)⁺; OR: −94.82

Compound 121: ¹H NMR: 400 MHz, DMSO-d₆ δ ppm 1.19-1.60 (m, 4H), 1.80-2.03 (m, 2H), 2.03-2.17 (m, 2H), 2.74 (br t, J=11.74 Hz, 1H), 3.56-3.82 (m, 1H), 3.99 (br d, J=7.58 Hz, 1H), 4.50 (t, J=11.25 Hz, 1H), 5.05 (br dd, J=11.13, 4.28 Hz, 1H), 6.72 (s, 2H), 7.18-7.29 (m, 3H), 7.30-7.37 (m, 2H), 7.45 (d, J=8.56 Hz, 2H), 7.55 (d, J=8.56 Hz, 2H), 7.70-7.82 (m, 4H); LCMS (ESI+): m/z 634.0 (M+H)⁺OR: 107.61

Compound 120: ¹H NMR: 400 MHz, DMSO-d₆ δ ppm 1.62 (br d, J=0.86 Hz, 2H), 1.74-1.98 (m, 6H), 2.82-3.00 (m, 1H), 3.85-4.28 (m, 2H), 4.52 (br t, J=11.37 Hz, 1H), 5.04 (br dd, J=10.88, 4.03 Hz, 1H), 6.79 (s, 2H), 7.19-7.29 (m, 3H), 7.29-7.37 (m, 2H), 7.43 (d, J=8.56 Hz, 2H), 7.56 (d, J=8.56 Hz, 2H), 7.60-7.73 (m, 3H), 7.80 (d, J=8.56 Hz, 2H); LCMS (ESI+): m/z 634.0 (M+H)⁺; OR: −67.00

Compound 122: ¹H NMR: ET51134-33-P4N1, 400 MHz, DMSO-d₆ δ ppm 1.52-1.71 (m, 2H), 1.75-2.05 (m, 6H), 2.81-3.03 (m, 1H), 3.75-4.32 (m, 2H), 4.53 (br t, J=11.31 Hz, 1H), 5.05 (br dd, J=11.07, 3.85 Hz, 1H), 6.80 (s, 2H), 7.20-7.29 (m, 3H), 7.30-7.37 (m, 2H), 7.44 (d, J=8.68 Hz, 2H), 7.57 (d, J=8.56 Hz, 2H), 7.61-7.73 (m, 3H), 7.81 (d, J=8.56 Hz, 2H); LCMS (ESI+): m/z 634.0 (M+H)⁺; OR: 64.20

### Synthesis of (S,Z)-N′-((4-chlorophenyl)sulfonyl)-3-(4-fluorophenyl)-4-phenyl-N-((1s,4R)-4-sulfamoylcyclohexyl)-4,5-dihydro-1H-pyrazole-1-carboximidamide (Compound 123), (R,Z)-N′-((4-chlorophenyl)sulfonyl)-3-(4-fluorophenyl)-4-phenyl-N-((1s,4S)-4-sulfamoylcyclohexyl)-4,5-dihydro-1H-pyrazole-1-carboximidamide (Compound 124), (S,Z)-N′-((4-chlorophenyl)sulfonyl)-3-(4-fluorophenyl)-4-phenyl-N-((1r,4S)-4-sulfamoylcyclohexyl)-4,5-dihydro-1H-pyrazole-1-carboximidamide (Compound 125) & (R,Z)-N′-((4-chlorophenyl)sulfonyl)-3-(4-fluorophenyl)-4-phenyl-N-((1 r,4R)-4-sulfamoylcyclohexyl)-4,5-dihydro-1H-pyrazole-1-carboximidamide (Compound 126)

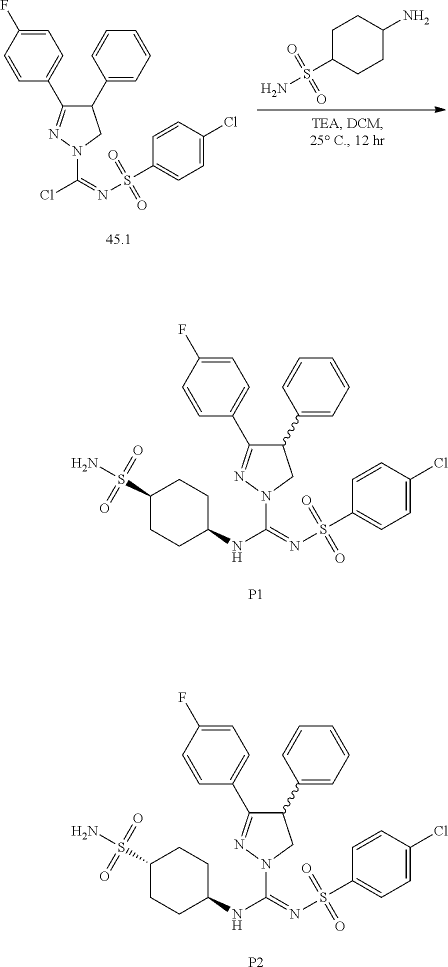 [SMILES: O=S(=O)(/N=C(/Cl)N1CC(c2ccccc2)C(c2ccc(F)cc2)=N1)c1ccc(Cl)cc1 ; NC1CCC(S(N)(=O)=O)CC1 ; NS(=O)(=O)[C@H]1CC[C@@H](N/C(=N/S(=O)(=O)c2ccc(Cl)cc2)N2CC(c3ccccc3)C(c3ccc(F)cc3)=N2)CC1 ; "P1" ; NS(=O)(=O)[C@H]1CC[C@H](N/C(=N/S(=O)(=O)c2ccc(Cl)cc2)N2CC(c3ccccc3)C(c3ccc(F)cc3)=N2)CC1 ; "P2"]

### Synthesis of P1 and P2; Compounds 123-126

To a solution of 45.1 (2 g, 4.33 mmol, 1 eq) in dichloromethane (20 mL) at rt were added triethylamine (3.01 mL, 21.65 mmol, 5 eq) and 4-aminocyclohexane-1-sulfonamide (926.67 mg, 4.33 mmol, 1 eq). After stirring at rt for 12 h, the mixture was concentrated under reduced pressure. The residue was purified by prep-HPLC to obtain racemic mixture P1 (450 mg, 673.66 μmol, 15.6%) as a white solid and racemic mixture P2 (200 mg, 315.16 μmol, 7.28%) as a white solid.

Method of prep-HPLC: Instrument: ACSTJ-GX-N; Column: Phenomenex luna C18 250×50 mm×10 μm; Mobile phase: A for H₂O=(0.04% HCl) and B for acetonitrile; Gradient: B from 60% to 80% in 10 min; Flow rate: 80 mL/min; Wavelength: 220&254 nm

P1: ¹H NMR: 400 MHz, DMSO-d₆ δ ppm 1.27-1.57 (m, 4H), 1.81-1.98 (m, 2H), 2.04-2.17 (m, 2H), 2.74 (br t, J=11.74 Hz, 1H), 3.53-3.82 (m, 1H), 3.91-4.02 (m, 1H), 4.50 (t, J=11.21 Hz, 1H), 5.06 (dd, J=11.09, 4.29 Hz, 1H), 6.72 (s, 2H), 7.18-7.37 (m, 7H), 7.50-7.59 (m, 2H), 7.74-7.83 (m, 4H) LCMS (ESI+): m/z 618.2 (M+H)⁺.

P2: ¹H NMR: 400 MHz, DMSO-d₆ δ ppm 1.54-1.71 (m, 2H), 1.75-2.00 (m, 7H), 2.82-2.98 (m, 1H), 3.90-4.03 (m, 1H), 4.52 (br t, J=11.38 Hz, 1H), 4.96-5.14 (m, 1H), 6.80 (s, 2H), 7.17-7.29 (m, 5H), 7.30-7.38 (m, 2H), 7.52-7.65 (m, 3H), 7.67-7.85 (m, 4H); LCMS (ESI+): m/z 618.2 (M+H)⁺.

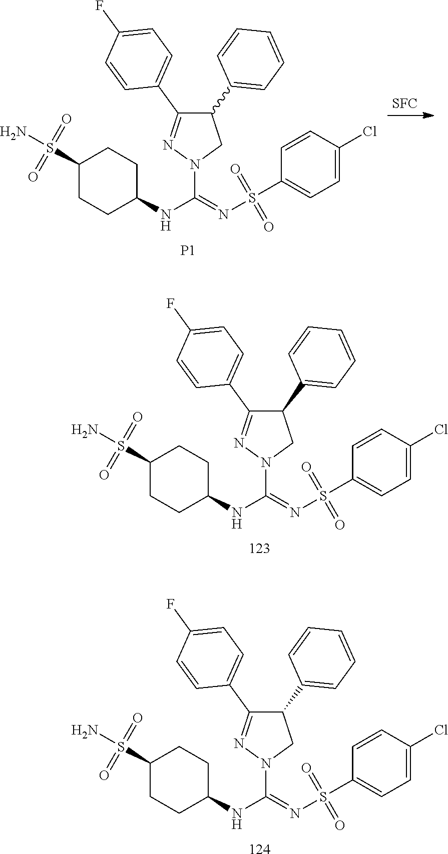

P1 (450 mg, 1.05 mmol, 1 eq) was separated by SFC to give Compound 123 (127 mg, 205.45 μmol, 28%) as a white solid and Compound 124 (121.2 mg, 196.07 μmol, 27%) as a white solid.

Method of SFC for separation of P1: Column: DAICEL CHIRALPAK AD (250 mm×30 mm, 10 μm); Mobile phase A: [NH₃H₂O (0.1%) in ethanol]Mobile phase B: CO2; B %: 50%-50%, Run time: 13 min. Absolute stereochemistry was not determined. Diastereomers with (-) OR were arbitrary assigned as S isomer.

Compound 123: ¹H NMR: 400 MHz, DMSO-d₆ δ ppm 1.33 (q, J=12.00 Hz, 2H), 1.41-1.59 (m, 2H), 1.91 (br d, J=12.40 Hz, 2H), 2.10 (br d, J=11.32 Hz, 2H), 2.74 (br t, J=11.86 Hz, 1H), 3.63-3.79 (m, 1H), 3.92-4.08 (m, 1H), 4.49 (t, J=11.21 Hz, 1H), 5.06 (dd, J=11.15, 4.35 Hz, 1H), 6.72 (s, 2H), 7.19-7.28 (m, 5H), 7.29-7.37 (m, 2H), 7.52-7.57 (m, 2H), 7.75-7.81 (m, 4H); LCMS (ESI+): m/z 618.0 (M+H)⁺; OR: −128.72

Compound 124: ¹H NMR: 400 MHz, DMSO-d₆ δ ppm 1.29-1.39 (m, 2H), 1.44-1.54 (m, 2H), 1.87-1.97 (m, 2H), 2.10 (br d, J=10.85 Hz, 2H), 2.74 (br t, J=11.80 Hz, 1H), 3.71 (br d, J=2.03 Hz, 1H), 3.99 (br d, J=7.51 Hz, 1H), 4.49 (br t, J=11.27 Hz, 1H), 5.06 (br dd, J=11.03, 4.23 Hz, 1H), 6.72 (s, 2H), 7.20-7.27 (m, 5H), 7.30-7.36 (m, 2H), 7.55 (d, J=8.46 Hz, 2H), 7.78 (br d, J=8.46 Hz, 4H); LCMS (ESI+): m/z 618.0 (M+H)⁺; OR: 123.08

P2 (200 mg, 1.05 mmol, 1 eq) was separated by SFC to give 125 (33.3 mg, 53.87 μmol, 17%) as a white solid and 126 (35 mg, 56.52 μmol, 18%) as a white solid.

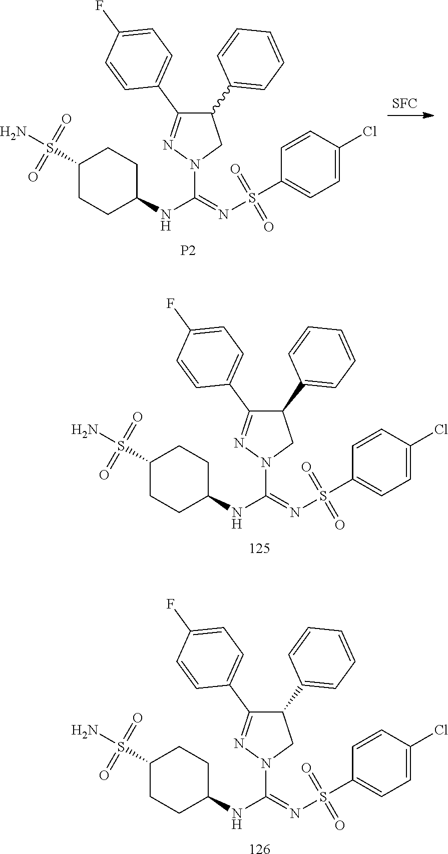

Method of SFC for separation of P2: Column: DAICEL CHIRALPAK AD (250 mm×30 mm, 10 μm); Mobile phase A: [NH₃H₂O (0.1%) in ethanol]; Mobile phase B: CO2; B %: 50%-50%, Run time: 15 min

Compound 125: ¹H NMR: 400 MHz, DMSO-d₆; δ ppm 1.63 (brd, J=2.86 Hz, 2H), 1.80-1.99 (m, 6H), 2.90 (dt, J=12.10, 6.23 Hz, 1H), 3.98 (br d, J=8.46 Hz, 1H), 4.51 (br t, J=11.27 Hz, 1H), 5.04 (br dd, J=10.91, 3.99 Hz, 1H), 6.79 (s, 2H), 7.18-7.27 (m, 5H), 7.30-7.35 (m, 2H), 7.56 (d, J=8.58 Hz, 2H), 7.61 (br d, J=6.68 Hz, 1H), 7.73 (br dd, J=8.23, 5.72 Hz, 2H), 7.80 (d, J=8.58 Hz, 2H); LCMS (ESI+): m/z 618.0 (M+H)⁺; OR: −150.19.

Compound 126: ¹H NMR: 400 MHz, DMSO-d₆ δ ppm 1.64 (br d, J=1.79 Hz, 2H), 1.80-2.00 (m, 6H), 2.85-2.96 (m, 1H), 3.98 (br d, J=7.51 Hz, 1H), 4.51 (br t, J=11.38 Hz, 1H), 5.04 (br dd, J=11.03, 3.99 Hz, 1H), 6.79 (s, 2H), 7.17-7.28 (m, 5H), 7.30-7.37 (m, 2H), 7.56 (d, J=8.46 Hz, 2H), 7.59-7.67 (m, 1H), 7.73 (br dd, J=8.05, 5.66 Hz, 2H), 7.80 (d, J=8.58 Hz, 2H); LCMS (ESI+): m/z 618.0 (M+H)⁺; OR: 137.16.

### Synthesis of (S,Z)-N′-((4-chlorophenyl)sulfonyl)-3-(4-fluorophenyl)-4-phenyl-N-((1r,3S)-3-sulfamoylcyclobutyl)-4,5-dihydro-1H-pyrazole-1-carboximidamide (Compound 127), (R,Z)-N′-((4-chlorophenyl)sulfonyl)-3-(4-fluorophenyl)-4-phenyl-N-((1r,3R)-3-sulfamoylcyclobutyl)-4,5-dihydro-1H-pyrazole-1-carboximidamide (Compound 128), (S,Z)-N′-((4-chlorophenyl)sulfonyl)-3-(4-fluorophenyl)-4-phenyl-N-((1s,3R)-3-sulfamoylcyclobutyl)-4,5-dihydro-1H-pyrazole-1-carboximidamide (Compound 129) & (R,Z)-N′-((4-chlorophenyl)sulfonyl)-3-(4-fluorophenyl)-4-phenyl-N-((1s,3S)-3-sulfamoylcyclobutyl)-4,5-dihydro-1H-pyrazole-1-carboximidamide (Compound 130)

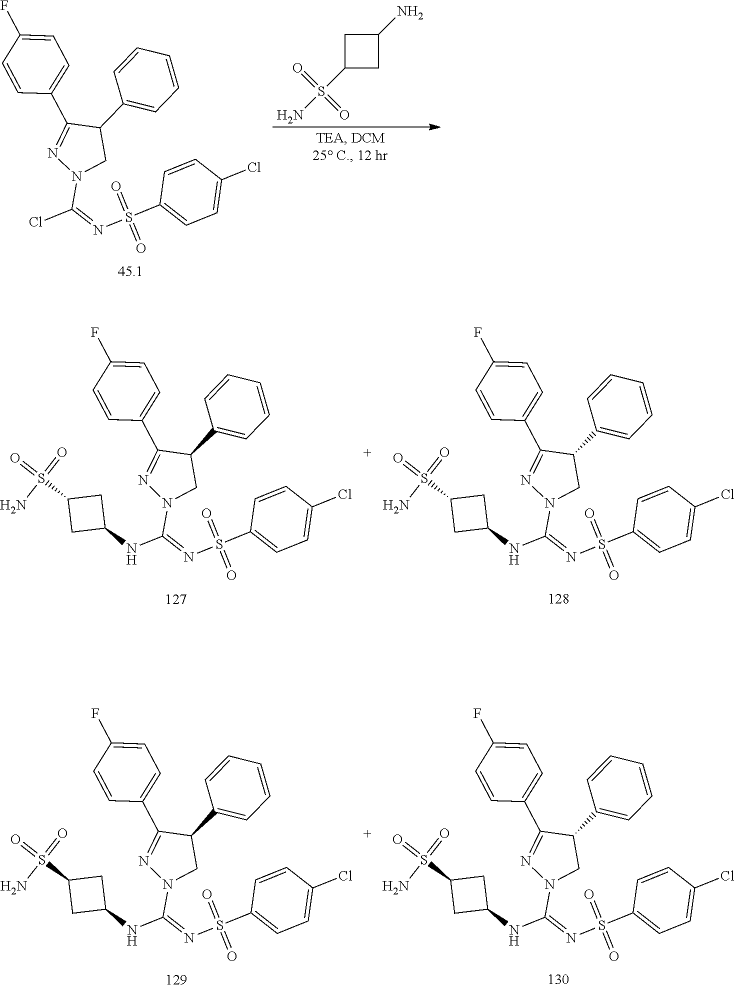

### Synthesis of Compounds 127-130

To a solution of 45.1 (1.5 g, 3.15 mmol, 1 eq) in dichloromethane (5 mL) at rt were added triethylamine (1.10 mL, 7.87 mmol, 2.5 eq) and 3-aminocyclobutane-1-sulfonamide (472.97 mg, 3.15 mmol, 1 eq). After stirring at rt for 12 h, the mixture was concentrated under reduced pressure. The residue was purified by column on silica gel using petroleum ether and ethyl acetate (1/1, v/v) to give (780 mg, 1.26 mmol, 40%) as a white solid. LCMS (ESI+): m/z 590.1 (M+H)⁺.

The diastereomers (780 mg, 1.32 mmol, 1 eq) were separated by the first SFC to give Compound 127 (85.8 mg, 0.14 mmol, 11%) as a white solid, Compound 129 (120.9 mg, 0.21 mmol, 16%) as a white solid and a racemic mixture of Compound 128 and Compound 130. The racemic mixture of Compound 128 and Compound 130 was further separated by the second SFC to give Compound 128 (70 mg, 0.12 mmol, 8%) as a white solid and Compound 130 (123.8 mg, 0.21 mmol, 15%) as a white solid. Absolute stereochemistry was not determined. Diastereomers with (-) OR were arbitrary assigned as S isomer.

Method of the first SFC: Column: REGIS (s,s) WHELK-01 (250 mm×30 mm, 10 μm); Mobile phase A: [NH₃H₂O (0.1%) in ethanol]; Mobile phase B: CO2; B %: 55%, Run time: 10 min

Method of the second SFC: Column: DAICEL CHIRALPAK AD (250 mm×30 mm, 10 μm); Mobile phase A: [NH₃H₂O (0.1%) in isopropanol]; Mobile phase B: CO2; B %: 50%, Run time: 12 min

Compound 127: ¹H NMR: 400 MHz, DMSO-d₆ δ ppm 2.29-2.44 (m, 3H), 2.54 (s, 1H), 3.41-3.52 (m, 1H), 3.94 (dd, J=11.25, 4.16 Hz, 1H), 4.24 (sxt, J=7.68 Hz, 1H), 4.47 (t, J=11.19 Hz, 1H), 5.05 (dd, J=11.13, 4.28 Hz, 1H), 6.80 (s, 2H), 7.14-7.40 (m, 7H), 7.48-7.61 (m, 2H), 7.70-7.86 (m, 4H), 8.10 (br d, J=5.87 Hz, 1H); LCMS (ESI+): m/z 590.0 (M+H)⁺; OR: −126.61.

Compound 129: ¹H NMR: 400 MHz, DMSO-d₆ δ ppm 2.07 (s, 1H), 2.55-2.74 (m, 4H), 3.50-3.67 (m, 1H), 3.96 (br dd, J=11.07, 3.97 Hz, 1H), 4.40-4.68 (m, 2H), 5.05 (br dd, J=11.07, 4.22 Hz, 1H), 6.86 (s, 2H), 7.15-7.39 (m, 7H), 7.53 (d, J=8.56 Hz, 2H), 7.69-7.91 (m, 4H), 8.16 (br d, J=5.75 Hz, 1H); LCMS (ESI+): m/z 590.0 (M+H)⁺; OR: −118.48.

Compound 128: ¹H NMR: 400 MHz, DMSO-d₆ δ ppm 2.30-2.48 (m, 4H), 3.46 (quin, J=8.41 Hz, 1H), 3.94 (br dd, J=11.13, 3.91 Hz, 1H), 4.15-4.33 (m, 1H), 4.47 (br t, J=11.25 Hz, 1H), 5.05 (br dd, J=11.13, 4.16 Hz, 1H), 6.80 (s, 2H), 7.16-7.38 (m, 7H), 7.55 (d, J=8.44 Hz, 2H), 7.73-7.86 (m, 4H), 8.10 (br d, J=4.65 Hz, 1H); LCMS (ESI+): m/z 590.0 (M+H)⁺; OR: 132.9.

Compound 130: ¹H NMR: 400 MHz, DMSO-d₆ δ ppm 2.07 (s, 1H), 2.53-2.73 (m, 4H), 3.52-3.66 (m, 1H), 3.96 (br dd, J=11.13, 4.03 Hz, 1H), 4.40-4.66 (m, 2H), 5.05 (dd, J=11.07, 4.22 Hz, 1H), 6.86 (s, 2H), 7.16-7.37 (m, 7H), 7.53 (d, J=8.56 Hz, 2H), 7.71-7.85 (m, 4H), 8.16 (br d, J=5.26 Hz, 1H); LCMS (ESI+): m/z 590.0 (M+H)⁺; OR: 126.48.

### Synthesis of (S)-1-((Z)-(((4-chlorophenyl)sulfonyl)imino)((S)-3-(4-fluorophenyl)-4-phenyl-4,5-dihydro-1H-pyrazol-1-yl)methyl)pyrrolidine-3-sulfonamide (Compound 131); (R)-1-((Z)-(((4-chlorophenyl)sulfonyl)imino)((S)-3-(4-fluorophenyl)-4-phenyl-4,5-dihydro-1H-pyrazol-1-yl)methyl)pyrrolidine-3-sulfonamide (Compound 132); (S)-1-((Z)-(((4-chlorophenyl)sulfonyl)imino)((R)-3-(4-fluorophenyl)-4-phenyl-4,5-dihydro-1H-pyrazol-1-yl)methyl)pyrrolidine-3-sulfonamide (Compound 133) & (R)-1-((Z)-(((4-chlorophenyl)sulfonyl)imino)((R)-3-(4-fluorophenyl)-4-phenyl-4,5-dihydro-1H-pyrazol-1-yl)methyl)pyrrolidine-3-sulfonamide (Compound 134)

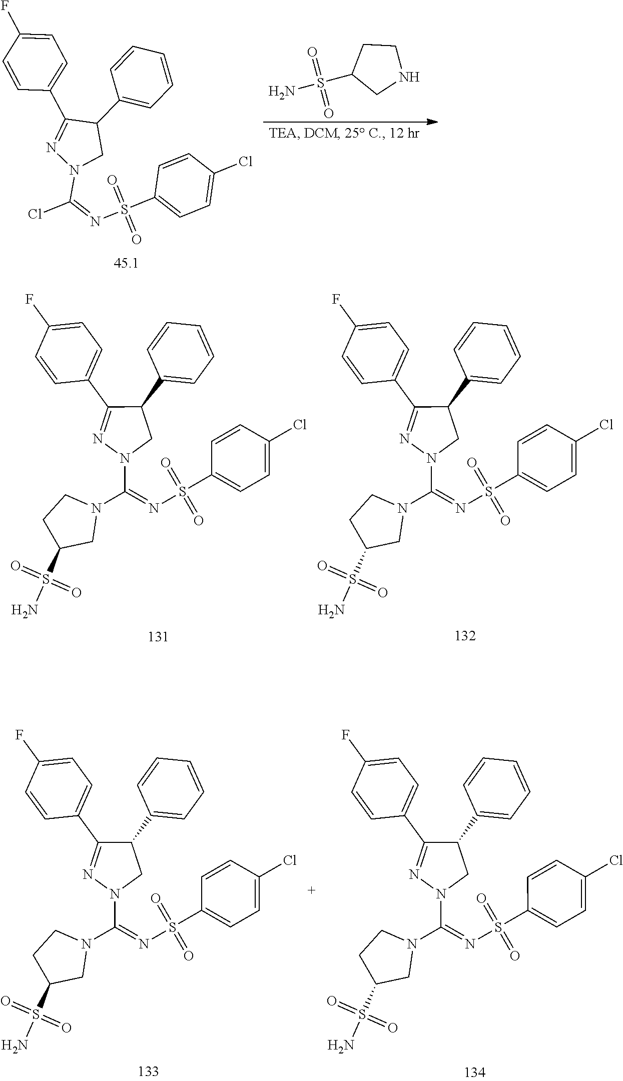

### Synthesis of Compounds 131-134

To a solution of 45.1 (1.5 g, 3.15 mmol, 1 eq) in dichloromethane (20 mL) at rt were added triethylamine (1.59 g, 15.74 mmol, 2.19 mL, 5 eq) and pyrrolidine-3-sulfonamide (472.97 mg, 3.15 mmol, 1 eq). After stirring at rt for 12 h, the mixture was concentrated under reduced pressure. The residue was purified by column on silica gel using petroleum ether and ethyl acetate (1/1, v/v) to give mixture of diastereomers (900 mg, 1.53 mmol, 44%) as a white solid.

The diastereomers (900 mg, 0.84 mmol, 1 eq) were separated by the first SFC to give Compound 131 (57.8 mg, 0.09 mmol, 11%) as a white solid, Compound 132 (62.1 mg, 0.10 mmol, 12%) as a white solid and a racemic mixture of 133 and 134. The resulting racemic mixture of Compound 133 and Compound 134 was further separated by the second SFC to obtain Compound 133 (48.1 mg, 0.08 mmol, 9%) as a white solid and Compound 134 (44.6 mg, 0.07 mmol, 8%) as a white solid. Absolute stereochemistry was not determined. Diastereomers with (-) OR were arbitrary assigned as S isomer.

Method of the first SFC: Column: Chiralpak IE-3, 50×4.6 mm I.D., 3 μm; Mobile phase: A: Hexane B: ethanol (0.1% isopropanol m) Gradient: A:B=60:40; Flow rate: 1 mL/min; Column temp: 30° C.

Method of the second SFC: Column: Chiralpak AD-3, 50×4.6 mm I.D., 3 μm; Mobile phase: A: CO₂ B: isopropanol (0.1% isopropanol m, v/v);Gradient: Time A % B % 0.0 95 5; 0.2 95 5; 1.2 50 50; 2.2 50 50; 2.6 95 5; 3.0 95 5; Flow rate: 3.4 mL/min; Column temp.: 35° C.; ABPR: 1800 psi.

Compound 131: ¹H NMR: 400 MHz, DMSO-d₆ δ ppm 2.18-2.28 (m, 2H) 3.71-3.85 (m, 3H) 3.95 (br d, J=7.13 Hz, 2H) 4.06 (dd, J=10.82, 4.94 Hz, 1H) 4.69 (t, J=11.19 Hz, 1H) 5.07 (dd, J=11.44, 4.94 Hz, 1H) 7.13 (s, 2H) 7.17-7.29 (m, 3H) 7.33 (d, J=4.38 Hz, 4H) 7.50-7.56 (m, 2H) 7.68 (dd, J=8.76, 5.50 Hz, 2H) 7.76-7.84 (m, 2H); LCMS (ESI+): m/z 590.0 (M+H)⁺; OR: −95.36.

Compound 132: ¹H NMR: 400 MHz, DMSO-d₆ δ ppm 2.20-2.31 (m, 2H) 3.63-3.73 (m, 1H) 3.74-3.82 (m, 1H) 3.84-3.96 (m, 2H) 3.97-4.12 (m, 2H) 4.69 (t, J=11.13 Hz, 1H) 5.06 (dd, J=11.38, 4.75 Hz, 1H) 7.14 (s, 2H) 7.17-7.28 (m, 3H) 7.30-7.35 (m, 4H) 7.50-7.58 (m, 2H) 7.66-7.71 (m, 2H) 7.78-7.84 (m, 2H); LCMS (ESI+): m/z 590.0 (M+1)⁺; OR: −111.20.

Compound 133: ¹H NMR: 400 MHz, DMSO-d₆ δ ppm 2.27 (q, J=7.00 Hz, 2H), 3.62-3.72 (m, 1H), 3.74-3.83 (m, 1H), 3.83-3.96 (m, 2H), 3.97-4.13 (m, 2H), 4.69 (t, J=11.13 Hz, 1H), 5.06 (dd, J=11.32, 4.69 Hz, 1H), 7.14 (s, 2H), 7.20 (t, J=8.88 Hz, 2H), 7.23-7.29 (m, 1H), 7.30-7.35 (m, 4H), 7.51-7.57 (m, 2H), 7.65-7.73 (m, 2H), 7.78-7.84 (m, 2H); LCMS (ESI+): m/z 590.0 (M+1)⁺; OR: 102.2.

Compound 134: ¹H NMR: 400 MHz, DMSO-d₆ δ ppm 2.23 (br dd, J=6.85, 3.67 Hz, 2H), 3.71-3.86 (m, 3H), 3.95 (br d, J=7.09 Hz, 2H), 4.07 (dd, J=10.82, 4.83 Hz, 1H), 4.69 (br t, J=11.13 Hz, 1H), 5.07 (dd, J=11.37, 4.89 Hz, 1H), 7.14 (s, 2H), 7.17-7.28 (m, 3H), 7.33 (d, J=4.40 Hz, 4H), 7.54 (d, J=8.56 Hz, 2H), 7.68 (dd, J=8.80, 5.50 Hz, 2H), 7.80 (d, J=8.56 Hz, 2H); LCMS (ESI+): m/z 590.0 (M+H)⁺; OR: 89.9.

### Synthesis of (S)-1-((Z)-((S)-3-(4-chlorophenyl)-4-phenyl-4,5-dihydro-1H-pyrazol-1-yl)(((4-chlorophenyl)sulfonyl)imino)methyl)pyrrolidine-3-sulfonamide (Compound 135); (R)-1-((Z)-((S)-3-(4-chlorophenyl)-4-phenyl-4,5-dihydro-1H-pyrazol-1-yl)(((4-chlorophenyl)sulfonyl)imino)methyl)pyrrolidine-3-sulfonamide (Compound 136); (R)-1-((Z)-((R)-3-(4-chlorophenyl)-4-phenyl-4,5-dihydro-1H-pyrazol-1-yl)(((4-chlorophenyl)sulfonyl)imino)methyl)pyrrolidine-3-sulfonamide (Compound 137) & (S)-1-((Z)-((R)-3-(4-chlorophenyl)-4-phenyl-4,5-dihydro-1H-pyrazol-1-yl)(((4-chlorophenyl)sulfonyl)imino)methyl)pyrrolidine-3-sulfonamide (Compound 138)

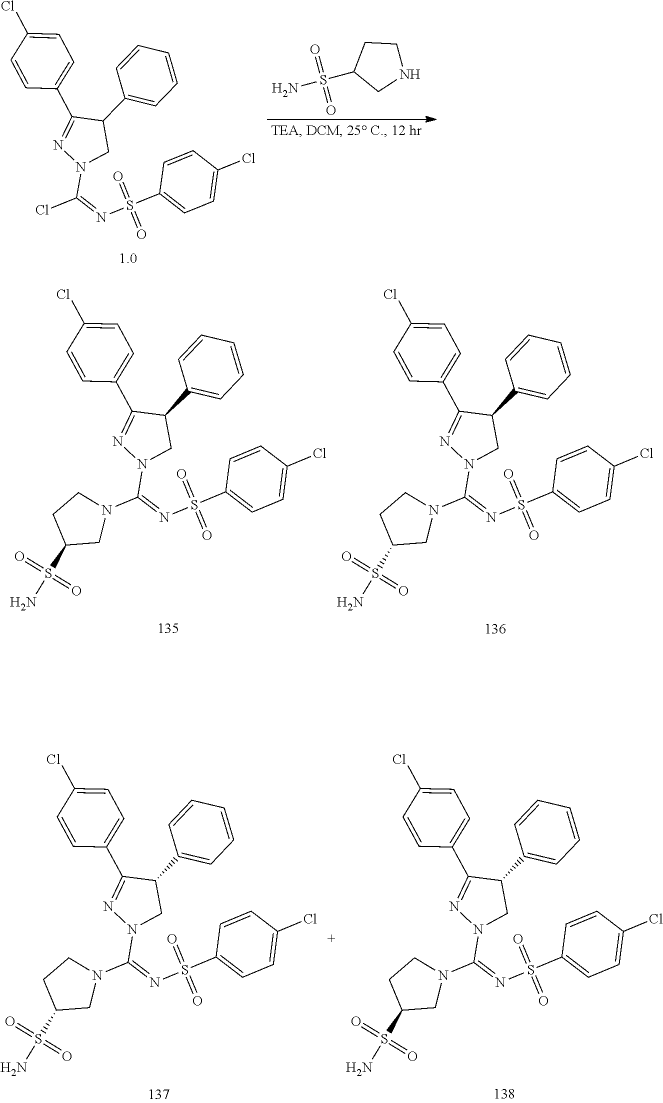

### Synthesis of Compounds 135-138

To a solution of 1.0 (2 g, 4.06 mmol, 1 eq) in dichloromethane (20 mL) at rt were added triethylamine (2.82 mL, 20.29 mmol, 5 eq) and pyrrolidine-3-sulfonamide (731.48 mg, 4.87 mmol, 1.2 eq). After stirring at rt for 12 h, the mixture was concentrated under reduced pressure. The residue was purified by column on silica gel using petroleum ether and ethyl acetate (1/1, v/v) to give mixture of diastereomers (400 mg, 659.48 μmol, 16%) as a white solid. LCMS (ESI+): m/z 606.1 (M+H)⁺.

The mixture of diastereomers (400 mg, 659.48 μmol, 1 eq) was separated by the first SFC to give a racemic mixture of Compounds 137 and 138, and a racemic mixture of Compounds 135 and 136. The racemic mixture of Compounds 137 and 138 was further separated by the second SFC to give Compound 137 (55.8 mg, 92.0 μmol, 13%) as a white solid and Compound 138 (52.5 mg, 86.56 μmol, 13%) as a white solid. The racemic mixture of Compounds 135 and 136 was further separated by the third SFC to give Compound 135 (48.9 mg, 80.62 μmol, 12%) as white solid and Compound 136 (56.5 mg, 93.15 μmol, 14%) as a white solid. Absolute stereochemistry was not determined. Diastereomers with (-) OR were arbitrary assigned as S isomer.

Method of the first SFC: Column: DAICEL CHIRALCEL OJ (250 mm*30 mm, 10 μm); Mobile phase A: [NH₃H₂O (0.1%) in ethanol]; Mobile phase B: CO2; B %: 50%-50%, Run time: 7 min.

Method of the second SFC: Column: REGIS (s,s) WHELK-01 (250 mm*30 mm,5 μm); Mobile phase A: [NH₃H₂O (0.1%) in ethanol]; Mobile phase B: CO2; B %: 50%-50%, Run time: 16 min.

Method of the third SFC: Column: DAICEL CHIRALPAK AD-H (250 mm*30 mm,5 μm); Mobile phase A: [NH₃H₂O (0.1%) in ACN/IPA]; Mobile phase B: CO2; B %: 50%-50%, Run time: 10 min.

Compound 135: ¹H NMR: 400 MHz, DMSO-d₆ δ ppm 2.15-2.29 (m, 2H), 3.67-3.86 (m, 3H), 3.88-4.01 (m, 2H), 4.08 (dd, J=10.88, 5.00 Hz, 1H), 4.70 (t, J=11.19 Hz, 1H), 5.07 (dd, J=11.44, 4.94 Hz, 1H), 7.14 (s, 2H), 7.21-7.29 (m, 1H), 7.30-7.37 (m, 4H), 7.42 (d, J=8.50 Hz, 2H), 7.54 (d, J=8.51 Hz, 2H), 7.63 (d, J=8.50 Hz, 2H), 7.81 (d, J=8.50 Hz, 2H); LCMS (ESI+): m/z 606.0 (M+H)⁺; OR: −76.18.

Compound 136: ¹H NMR: 400 MHz, DMSO-d₆ δ ppm 2.27 (q, J=6.92 Hz, 2H), 3.62-3.72 (m, 1H), 3.78 (quin, J=6.28 Hz, 1H), 3.83-3.95 (m, 2H), 3.98-4.11 (m, 2H), 4.70 (t, J=11.19 Hz, 1H), 5.07 (dd, J=11.51, 4.75 Hz, 1H), 7.14 (s, 2H), 7.25 (td, J=5.35, 3.44 Hz, 1H), 7.29-7.36 (m, 4H), 7.42 (d, J=8.63 Hz, 2H), 7.54 (d, J=8.50 Hz, 2H), 7.63 (d, J=8.50 Hz, 2H), 7.81 (d, J=8.50 Hz, 2H); LCMS (ESI+): m/z 606.0 (M+H)⁺; OR: −98.21.

Compound 137: ¹H NMR: 400 MHz, DMSO-d₆ δ ppm 2.27 (q, J=7.00 Hz, 2H), 3.61-3.72 (m, 1H), 3.74-3.82 (m, 1H), 3.83-3.95 (m, 2H), 3.97-4.11 (m, 2H), 4.70 (t, J=11.19 Hz, 1H), 5.06 (dd, J=11.44, 4.82 Hz, 1H), 7.14 (s, 2H), 7.25 (td, J=5.50, 3.25 Hz, 1H), 7.29-7.36 (m, 4H), 7.42 (d, J=8.63 Hz, 2H), 7.51-7.57 (m, 2H), 7.63 (d, J=8.63 Hz, 2H), 7.78-7.83 (m, 2H); LCMS (ESI+): m/z 606.0 (M+H)⁺; OR: 90.61.

Compound 138: ¹H NMR: 400 MHz, DMSO-d₆ δ ppm 2.17-2.29 (m, 2H), 3.71-3.86 (m, 3H), 3.94 (br d, J=7.13 Hz, 2H), 4.07 (dd, J=10.88, 5.00 Hz, 1H), 4.70 (br t, J=11.26 Hz, 1H), 5.07 (dd, J=11.38, 4.88 Hz, 1H), 7.14 (s, 2H), 7.22-7.29 (m, 1H), 7.30-7.36 (m, 4H), 7.42 (d, J=8.63 Hz, 2H), 7.54 (d, J=8.50 Hz, 2H), 7.63 (d, J=8.50 Hz, 2H), 7.81 (d, J=8.50 Hz, 2H); LCMS (ESI+): m/z 606.0 (M+H)⁺; OR: 77.98.

### COMPOUNDS

The absolute stereochemistry 3-aminocyclopentane-1-sulfonamide was arbitrarily assigned post separation of the four diastereomers. As such, the absolute stereochemistry of the Compounds 139-146 is arbitrarily assigned. Diastereomers with (-) OR were arbitrary assigned as S isomer.

### Synthesis of (S,Z)-3-(4-chlorophenyl)-N′-((4-chlorophenyl)sulfonyl)-4-phenyl-N-((1S,3S)-3-sulfamoylcyclopentyl)-4,5-dihydro-1H-pyrazole-1-carboximidamide (Compound 139); (R,Z)-3-(4-chlorophenyl)-N′-((4-chlorophenyl)sulfonyl)-4-phenyl-N-((1S,3S)-3-sulfamoylcyclopentyl)-4,5-dihydro-1H-pyrazole-1-carboximidamide (Compound 140)

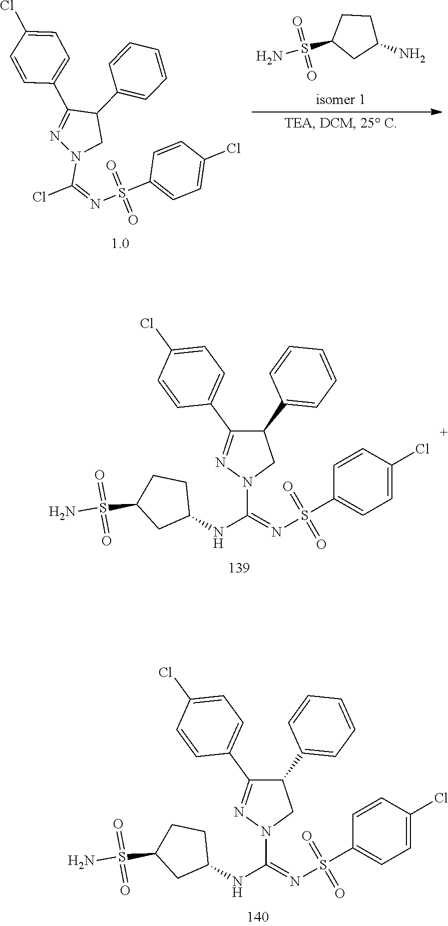

### Synthesis of Compounds 139 and 140

To a solution of 1.0 (180.05 mg, 365.35 μmol, 1.5 eq) in dichloromethane (1 mL) at rt were added triethylamine (203.41 μL, 1.46 mmol, 6 eq) and (1R,3S)-3-aminocyclopentane-1-sulfonamide (40 mg, 243.57 μmol, 1 eq). After stirring at rt for 12 h, the mixture was concentrated under reduced pressure. The residue was purified by column on silica gel using petroleum ether and ethyl acetate (1/2, v/v) to give a crude, which was further separated by SFC to give Compound 139 (5.8 mg, 9.35 μmol, 3%) as a white solid and Compound 140 (6.6 mg, 10.44 μmol, 4%) as a white solid.

Compound 139: ¹H NMR: 400 MHz, DMSO-d₆ δ ppm 7.85-7.70 (m, 5H), 7.58-7.51 (m, 2H), 7.44 (d, J=8.6 Hz, 2H), 7.38-7.29 (m, 2H), 7.28-7.18 (m, 3H), 6.80 (s, 2H), 5.06 (dd, J=4.3, 11.1 Hz, 1H), 4.55 (t, J=11.1 Hz, 1H), 4.47-4.35 (m, 1H), 4.00 (br dd, J=3.5, 10.9 Hz, 1H), 3.66-3.52 (m, 1H), 2.35-2.23 (m, 1H), 2.11-1.90 (m, 3H), 1.88-1.77 (m, 1H), 1.73-1.58 (m, 1H). LCMS (ESI+): m/z 620.0 (M+H)⁺; OR: −100.44.

Compound 140: ¹H NMR: 400 MHz, DMSO-d₆ δ ppm 7.83-7.68 (m, 4H), 7.54 (d, J=8.5 Hz, 2H), 7.44 (d, J=8.5 Hz, 2H), 7.37-7.29 (m, 2H), 7.28-7.17 (m, 3H), 6.80 (s, 2H), 5.10-5.03 (m, 1H), 4.57-4.47 (m, 1H), 4.45-4.33 (m, 1H), 4.10-4.01 (m, 1H), 3.62-3.53 (m, 1H), 2.27-2.21 (m, 1H), 2.08-1.94 (m, 3H), 1.87-1.78 (m, 1H), 1.75-1.66 (m, 1H); LCMS (ESI+): m/z 620.0 (M+H)⁺; OR: 103.39.

### Synthesis of (S,Z)-3-(4-chlorophenyl)-N′-((4-chlorophenyl)sulfonyl)-4-phenyl-N-((1R,3R)-3-sulfamoylcyclopentyl)-4,5-dihydro-1H-pyrazole-1-carboximidamide (141) & (R,Z)-3-(4-chlorophenyl)-N′-((4-chlorophenyl)sulfonyl)-4-phenyl-N-((1R,3R)-3-sulfamoylcyclopentyl)-4,5-dihydro-1H-pyrazole-1-carboximidamide (142)

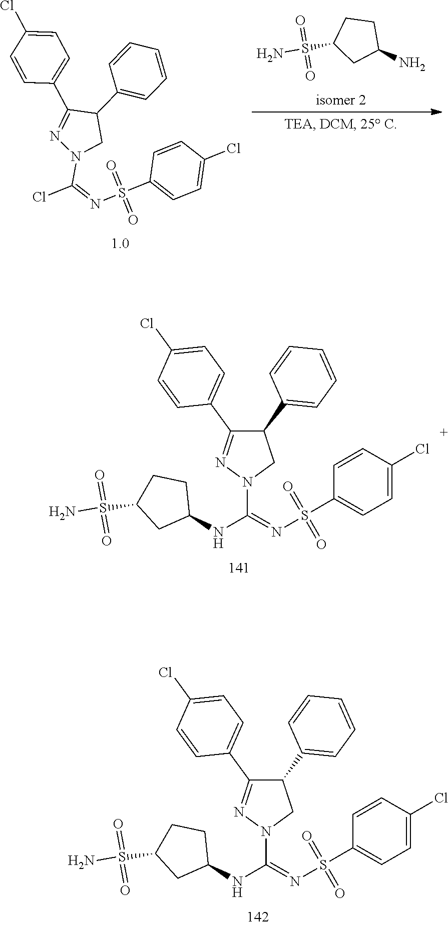

### Synthesis of Compounds 141 and 142

To a solution of 1.0 (247.56 mg, 502.36 μmol, 1.5 eq) in dichloromethane (1 mL) at rt were added triethylamine (279.69 μL, 2.01 mmol, 6 eq) and (1S,3R)-3-aminocyclopentane-1-sulfonamide (55 mg, 334.91 μmol, 1 eq). After stirring at rt for 12 h, the mixture was concentrated under reduced pressure. The residue was purified by column on silica gel using petroleum ether and ethyl acetate (1/2, v/v) to give a racemate, which was further separated by SFC to give Compound 141 (38.7 mg, 22.18 μmol, 18%) as a white solid and Compound 142 (39 mg, 21.22 μmol, 18%) as a white solid.

Compound 141: ¹H NMR: 400 MHz, DMSO-d₆ δ ppm 7.94 (br s, 1H) 7.72-7.83 (m, 4H) 7.54 (d, J=7.76 Hz, 2H) 7.44 (d, J=8.58 Hz, 2H) 7.19-7.36 (m, 5H) 6.89 (s, 2H) 5.07 (dd, J=11.15, 4.23 Hz, 1H) 4.52 (t, J=11.27 Hz, 1H) 4.27 (br s, 1H) 3.90-4.10 (m, 1H) 3.41-3.51 (m, 1H) 2.21-2.38 (m, 1H) 1.82-2.08 (m, 4H) 1.66-1.79 (m, 1H); LCMS (ESI+): m/z 620.0 (M+H)⁺; OR: −107.40.

Compound 142: ¹H NMR: 400 MHz, DMSO-d₆ δ ppm 7.95 (br s, 1H) 7.68-7.86 (m, 4H) 7.55 (d, J=8.58 Hz, 2H) 7.44 (d, J=8.58 Hz, 2H) 7.30-7.39 (m, 2H) 7.19-7.29 (m, 3H) 6.88 (s, 2H) 5.07 (br dd, J=11.15, 4.35 Hz, 1H) 4.50 (br t, J=11.21 Hz, 1H) 4.28 (br d, J=4.65 Hz, 1H) 3.97-4.11 (m, 1H) 3.41-3.50 (m, 1H) 2.19-2.36 (m, 1H) 1.71-2.09 (m, 5H); LCMS (ESI+): m/z 620.0 (M+H)⁺; OR: 82.50.

### Synthesis of (S,Z)-3-(4-chlorophenyl)-N′-((4-chlorophenyl)sulfonyl)-4-phenyl-N-((1S,3R)-3-sulfamoylcyclopentyl)-4,5-dihydro-1H-pyrazole-1-carboximidamide (Compound 143) & (R,Z)-3-(4-chlorophenyl)-N′-((4-chlorophenyl)sulfonyl)-4-phenyl-N-((1S,3R)-3-sulfamoylcyclopentyl)-4,5-dihydro-1H-pyrazole-1-carboximidamide (Compound 144)

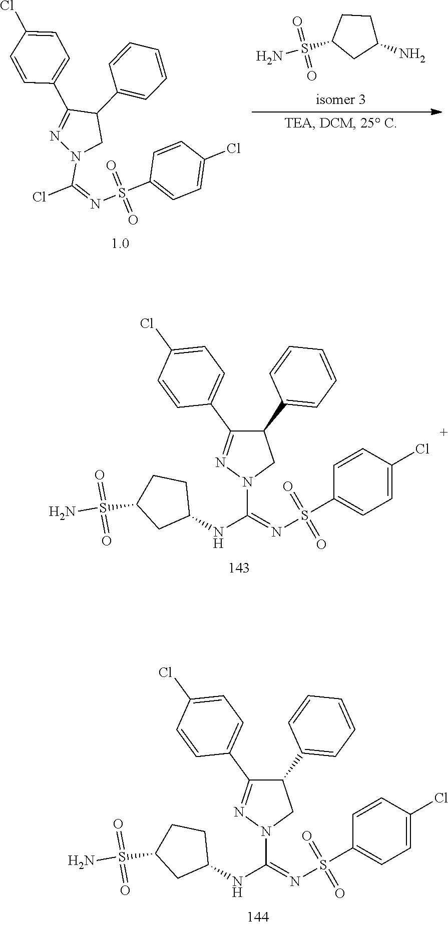

### Synthesis of Compounds 143 and 144

To a solution of 1.0 (382.60 mg, 776.37 μmol, 1.5 eq) in dichloromethane (2 mL) at rt were added triethylamine (432.25 μL, 3.11 mmol, 6 eq) and (1S,3S)-3-aminocyclopentane-1-sulfonamide (85 mg, 517.58 μmol, 1 eq). After stirring at rt for 12 h, the mixture was concentrated under reduced pressure. The residue was purified by column on silica gel using petroleum ether and ethyl acetate (1/2, v/v) to give a racemate, which was further separated by SFC to give Compound 143 (78.3 mg, 130.55 μmol, 24.3%) as a white solid and Compound 144 (68.7 mg, 120.85 μmol, 21.4%) as a white solid.

Compound 143: ¹H NMR: 400 MHz, DMSO-d₆ δ ppm 7.68-7.89 (m, 5H) 7.54 (br d, J=8.34 Hz, 2H) 7.44 (br d, J=8.34 Hz, 2H) 7.20-7.36 (m, 5H) 6.81 (br s, 2H) 4.97-5.16 (m, 1H) 4.31-4.60 (m, 2H) 4.06 (br d, J=7.87 Hz, 1H) 3.52-3.63 (m, 1H) 2.25 (dt, J=13.20, 6.57 Hz, 1H) 1.94-2.12 (m, 3H) 1.78-1.91 (m, 1H) 1.64-1.77 (m, 1H); LCMS (ESI+): m/z 620.0 (M+H)⁺; OR: −100.60.

Compound 144: ¹H NMR: 400 MHz DMSO-d₆ δ ppm 7.66-7.89 (m, 5H) 7.54 (br d, J=8.34 Hz, 2H) 7.44 (br d, J=8.23 Hz, 2H) 7.16-7.39 (m, 5H) 6.81 (br s, 2H) 5.06 (br d, J=7.39 Hz, 1H) 4.55 (br t, J=11.27 Hz, 1H) 4.42 (br s, 1H) 4.00 (br d, J=8.34 Hz, 1H) 3.54-3.66 (m, 1H) 2.22-2.37 (m, 1H) 1.91-2.11 (m, 3H) 1.77-1.91 (m, 1H) 1.57-1.75 (m, 1H); LCMS (ESI+): m/z 620.0 (M+1)⁺; OR: 90.60.

### Synthesis of (S,Z)-3-(4-chlorophenyl)-N′-((4-chlorophenyl)sulfonyl)-4-phenyl-N-((1R,3S)-3-sulfamoylcyclopentyl)-4,5-dihydro-1H-pyrazole-1-carboximidamide (Compound 145) & (R,Z)-3-(4-chlorophenyl)-N′-((4-chlorophenyl)sulfonyl)-4-phenyl-N-((1R,3S)-3-sulfamoylcyclopentyl)-4,5-dihydro-1H-pyrazole-1-carboximidamide (Compound 146)

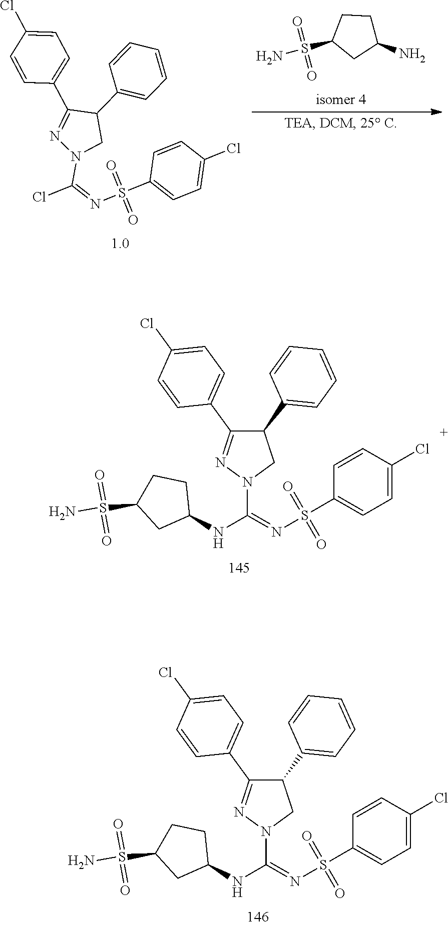

### Synthesis of Compounds 145 and 146

To a solution of 1.0 (211.56 mg, 429.29 μmol, 1.5 eq) in dichloromethane (1 mL) at rt were added triethylamine (239.01 μL, 1.72 mmol, 6 eq) and (1R,3R)-3-aminocyclopentane-1-sulfonamide (47 mg, 286.19 μmol, 1 eq). After stirring at rt for 12 h, the mixture was concentrated under reduced pressure. The residue was purified by column on silica gel using petroleum ether and ethyl acetate (1/2, v/v) to give racemate, which was further separated by SFC to give Compound 145 (20.6 mg, 32.3 μmol, 11%) as a white solid and Compound 146 (14.2 mg, 22.52 μmol, 7%) as a white solid.

Compound 145: ¹H NMR: 400 MHz, DMSO-d₆ δ ppm 7.95 (br d, J=1.2 Hz, 1H), 7.83-7.78 (m, 2H), 7.74 (d, J=8.6 Hz, 2H), 7.58-7.52 (m, 2H), 7.44 (d, J=8.7 Hz, 2H), 7.37-7.30 (m, 2H), 7.28-7.20 (m, 3H), 6.88 (s, 2H), 5.07 (dd, J=4.4, 11.1 Hz, 1H), 4.51 (t, J=11.2 Hz, 1H), 4.35-4.22 (m, 1H), 4.11-3.97 (m, 1H), 3.52-3.40 (m, 1H), 2.36-2.22 (m, 1H), 2.12-1.83 (m, 4H), 1.81-1.70 (m, 1H); LCMS (ESI+): m/z 620.0 (M+H)⁺; OR:−157.42

Compound 146: ¹H NMR: 400 MHz DMSO-d₆ δ ppm 8.03-7.87 (m, 1H), 7.84-7.70 (m, 4H), 7.54 (d, J=8.5 Hz, 2H), 7.44 (d, J=8.7 Hz, 2H), 7.38-7.29 (m, 2H), 7.28-7.17 (m, 3H), 6.89 (s, 2H), 5.07 (br dd, J=4.3, 11.2 Hz, 1H), 4.52 (t, J=11.3 Hz, 1H), 4.37-4.20 (m, 1H), 4.07-3.94 (m, 1H), 3.53-3.39 (m, 1H), 2.37-2.22 (m, 1H), 2.10-1.96 (m, 2H), 1.94-1.68 (m, 3H); LCMS (ESI+): m/z 620.0 (M+H)⁺; OR:78.53.

### Synthesis of (S,Z)-N′-((4-chlorophenyl)sulfonyl)-3-(4-fluorophenyl)-4-phenyl-N-((1R,3S)-3-sulfamoylcyclopentyl)-4,5-dihydro-1H-pyrazole-1-carboximidamide, (R,Z)-N′-((4-chlorophenyl)sulfonyl)-3-(4-fluorophenyl)-4-phenyl-N-((1R,3S)-3-sulfamoylcyclopentyl)-4,5-dihydro-1H-pyrazole-1-carboximidamide, (S,Z)-N′-((4-chlorophenyl)sulfonyl)-3-(4-fluorophenyl)-4-phenyl-N-((1S,3R)-3-sulfamoylcyclopentyl)-4,5-dihydro-1H-pyrazole-1-carboximidamide, (R,Z)-N′-((4-chlorophenyl)sulfonyl)-3-(4-fluorophenyl)-4-phenyl-N-((1S,3R)-3-sulfamoylcyclopentyl)-4,5-dihydro-1H-pyrazole-1-carboximidamide, (S,Z)-N′-((4-chlorophenyl)sulfonyl)-3-(4-fluorophenyl)-4-phenyl-N-((1S,3S)-3-sulfamoylcyclopentyl)-4,5-dihydro-1H-pyrazole-1-carboximidamide, (R,Z)-N′-((4-chlorophenyl)sulfonyl)-3-(4-fluorophenyl)-4-phenyl-N-((1S,3S)-3-sulfamoylcyclopentyl)-4,5-dihydro-1H-pyrazole-1-carboximidamide, (S,Z)-N′-((4-chlorophenyl)sulfonyl)-3-(4-fluorophenyl)-4-phenyl-N-((1R,3R)-3-sulfamoylcyclopentyl)-4,5-dihydro-1H-pyrazole-1-carboximidamide, (R,Z)-N′-((4-chlorophenyl)sulfonyl)-3-(4-fluorophenyl)-4-phenyl-N-((1R,3R)-3-sulfamoylcyclopentyl)-4,5-dihydro-1H-pyrazole-1-carboximidamide (Compounds 147-154)

The absolute stereochemistry 3-aminocyclopentane-1-sulfonamide was arbitrarily assigned post separation of the four diastereomers. As such, the absolute stereochemistry of the following examples is arbitrarily assigned. Diastereomers with (-) OR were arbitrary assigned as S isomer. Starting material for the following examples is 45.1.

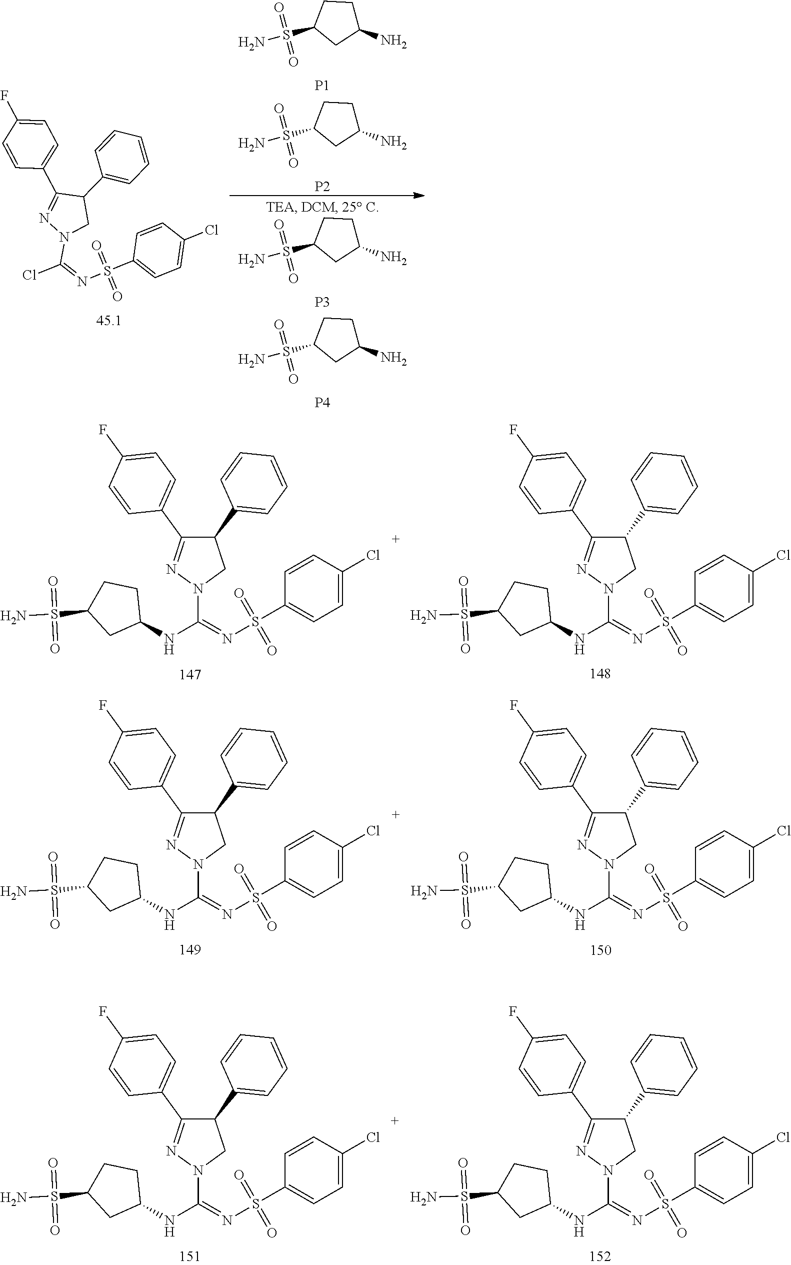

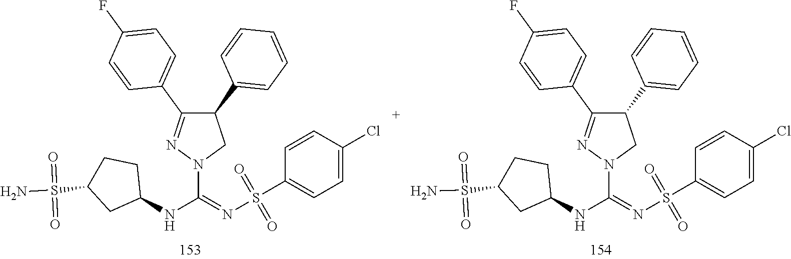

Compound 147: ¹H NMR: 400 MHz, DMSO-d₆ δ ppm 7.87-7.73 (m, 5H), 7.54 (d, J=8.4 Hz, 2H), 7.35-7.29 (m, 2H), 7.28-7.16 (m, 5H), 6.81 (s, 2H), 5.06 (br dd, J=4.1, 11.1 Hz, 1H), 4.54 (br t, J=11.2 Hz, 1H), 4.42 (br s, 1H), 4.00 (br dd, J=3.2, 10.7 Hz, 1H), 3.60 (quin, J=7.8 Hz, 1H), 2.29 (td, J=7.0, 13.6 Hz, 1H), 2.09-1.90 (m, 3H), 1.89-1.77 (m, 1H), 1.72-1.60 (m, 1H); LCMS (ESI+): m/z 604.0 (M+H)⁺; OR: −121.90

Compound 148: ¹H NMR: 400 MHz, DMSO-d₆ δ ppm 7.87-7.69 (m, 5H), 7.54 (d, J=8.6 Hz, 2H), 7.38-7.29 (m, 2H), 7.29-7.16 (m, 5H), 6.81 (s, 2H), 5.07 (dd, J=4.2, 11.1 Hz, 1H), 4.51 (t, J=11.2 Hz, 1H), 4.42 (br d, J=3.8 Hz, 1H), 4.07 (br d, J=7.8 Hz, 1H), 3.64-3.53 (m, 1H), 2.25 (td, J=6.9, 13.5 Hz, 1H), 2.12-1.92 (m, 3H), 1.90-1.78 (m, 1H), 1.76-1.65 (m, 1H); LCMS (ESI+): m/z 604.0 (M+H)⁺, OR: 129.60

Compound 149: ¹H NMR: 400 MHz, DMSO-d₆ δ ppm 7.92 (br s, 1H), 7.79 (br d, J=8.6 Hz, 4H), 7.54 (d, J=8.4 Hz, 2H), 7.37-7.30 (m, 2H), 7.28-7.18 (m, 5H), 6.89 (br s, 2H), 5.07 (br dd, J=4.0, 11.1 Hz, 1H), 4.51 (br t, J=11.2 Hz, 1H), 4.28 (br s, 1H), 4.00 (br d, J=8.1 Hz, 1H), 3.53-3.40 (m, 1H), 2.38-2.24 (m, 1H), 2.09-1.97 (m, 2H), 1.96-1.79 (m, 2H), 1.77-1.66 (m, 1H); LCMS (ESI+): m/z 604.0 (M+H)⁺; OR: −108.40

Compound 150: ¹H NMR: 400 MHz, DMSO-d₆ δ ppm 7.93 (br s, 1H), 7.84-7.75 (m, 4H), 7.54 (d, J=8.6 Hz, 2H), 7.39-7.30 (m, 2H), 7.28-7.17 (m, 5H), 6.88 (s, 2H), 5.07 (br dd, J=4.2, 11.0 Hz, 1H), 4.49 (t, J=11.2 Hz, 1H), 4.28 (br d, J=5.7 Hz, 1H), 4.11-3.96 (m, 1H), 3.54-3.39 (m, 1H), 2.36-2.19 (m, 1H), 2.11-1.71 (m, 5H); LCMS (ESI+): m/z 604.0 (M+H)⁺; OR: 118.60

Compound 151: ¹H NMR: 400 MHz, DMSO-d₆ δ ppm 7.92 (br s, 1H), 7.79 (br d, J=8.6 Hz, 4H), 7.54 (d, J=8.4 Hz, 2H), 7.37-7.30 (m, 2H), 7.28-7.18 (m, 5H), 6.89 (br s, 2H), 5.07 (br dd, J=4.0, 11.1 Hz, 1H), 4.51 (br t, J=11.2 Hz, 1H), 4.28 (br s, 1H), 4.00 (br d, J=8.1 Hz, 1H), 3.53-3.40 (m, 1H), 2.38-2.24 (m, 1H), 2.09-1.97 (m, 2H), 1.96-1.79 (m, 2H), 1.77-1.66 (m, 1H); LCMS (ESI+): m/z 604.0 (M+H)⁺; OR: −124.80

Compound 152: ¹H NMR: 400 MHz DMSO-d₆ δ ppm 7.87-7.70 (m, 5H), 7.54 (d, J=8.6 Hz, 2H), 7.35-7.29 (m, 2H), 7.28-7.17 (m, 5H), 6.81 (br s, 2H), 5.06 (br dd, J=4.1, 11.2 Hz, 1H), 4.54 (br t, J=11.2 Hz, 1H), 4.42 (br s, 1H), 4.00 (br dd, J=3.5, 10.9 Hz, 1H), 3.67-3.52 (m, 1H), 2.29 (td, J=7.0, 13.8 Hz, 1H), 2.09-1.90 (m, 3H), 1.88-1.77 (m, 1H), 1.65 (qd, J=8.1, 11.9 Hz, 1H); LCMS (ESI+): m/z 604.0 (M+H)⁺; OR:119.60

Compound 153: ¹H NMR: 400 MHz, DMSO-d₆ δ 7.94 (br d, J=5.1 Hz, 1H), 7.82-7.75 (m, 4H), 7.54 (d, J=8.5 Hz, 2H), 7.36-7.30 (m, 2H), 7.28-7.18 (m, 5H), 6.88 (br s, 2H), 5.07 (dd, J=4.1, 11.0 Hz, 1H), 4.49 (t, J=11.2 Hz, 1H), 4.28 (br s, 1H), 4.04 (br dd, J=3.1, 10.5 Hz, 1H), 3.52-3.41 (m, 1H), 2.35-2.21 (m, 1H), 2.09-1.83 (m, 4H), 1.80-1.68 (m, 1H); LCMS (ESI+): m/z 604.0 (M+H)⁺; OR:−123.17

Compound 154: ¹H NMR: 400 MHz DMSO-d₆ δ ppm 7.92 (br d, J=3.3 Hz, 1H), 7.84-7.75 (m, 4H), 7.54 (d, J=8.6 Hz, 2H), 7.37-7.30 (m, 2H), 7.28-7.18 (m, 5H), 6.89 (s, 2H), 5.07 (dd, J=4.2, 11.0 Hz, 1H), 4.51 (t, J=11.1 Hz, 1H), 4.28 (td, J=2.0, 4.2 Hz, 1H), 4.07-3.92 (m, 1H), 3.56-3.41 (m, 1H), 2.39-2.23 (m, 1H), 2.09-1.97 (m, 2H), 1.95-1.67 (m, 3H); LCMS (ESI+): m/z 604.1 (M+H)⁺; OR:106.82

### Synthesis of (S,E)-3-(4-chlorophenyl)-N′-((4-chlorophenyl)sulfonyl)-N-(2-(N,N-dimethylsulfamoyl)ethyl)-4-phenyl-4,5-dihydro-1H-pyrazole-1-carboximidamide (Compound 155) & (R,E)-3-(4-chlorophenyl)-N′-((4-chlorophenyl)sulfonyl)-N-(2-(N,N-dimethylsulfamoyl)ethyl)-4-phenyl-4,5-dihydro-1H-pyrazole-1-carboximidamide (Compound 156)

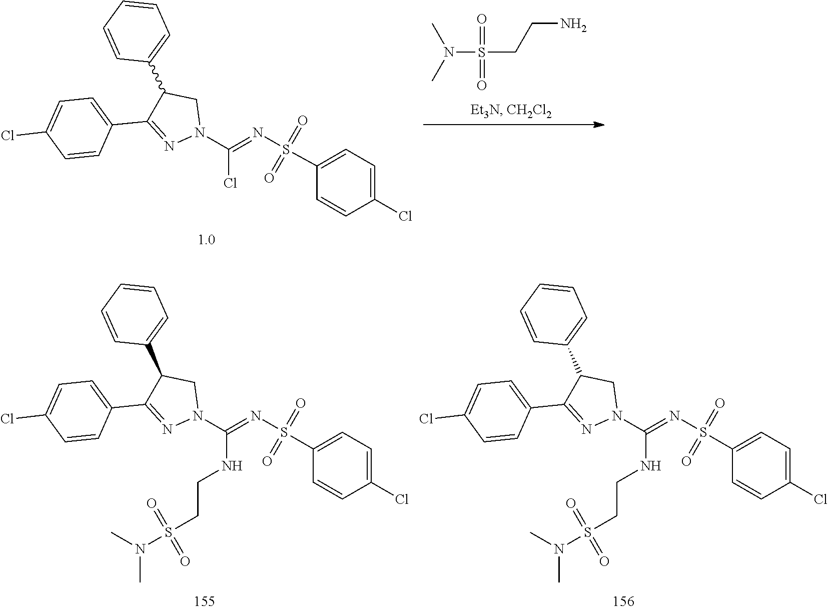

### Synthesis of Compounds 155 and 156

To a solution of 2-amino-N,N-dimethylethane-1-sulfonamide (85 mg, 0.53 mmol, 1.1 eq.) and triethylamine (0.21 mL, 1.53 mmol, 3.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0). After stirring at rt for 18 h, the reaction was diluted with dichloromethane (25 mL) and washed with aqueous HCl (2N, 20 mL). The organic layer was dried over magnesium sulfate, filtered, and concentrated in vacuo. The residue was purified by flash chromatography on silica eluting with methanol/dichloromethane mixtures (0/100 to 10/90, v/v) to give a colorless oil, which was further subjected to SFC separation conditions, obtaining the following enantiomers: Compound 155 (76.9 mg, 0.13 mmol, 25% yield, 97% purity, 99% ee, OR: −96.3) and Compound 156 (33.6 mg, 0.056 mol, 11% yield, 99% purity, 99% ee, OR: +113.9).

Method of SFC: SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%) iPrOH+0.1% diethylamine, at 35° C., BPR 100 bar. Acquisition frequency was set to 220 nm for the DAD detector.

Compound 155: ¹H NMR (DMSO-d6, 400 MHz) δ 8.22 (br s, 1H), 7.8-7.9 (m, 2H), 7.7-7.8 (m, 2H), 7.5-7.6 (m, 2H), 7.4-7.5 (m, 2H), 7.3-7.4 (m, 2H), 7.2-7.3 (m, 3H), 5.10 (dd, J=4.4, 11.2 Hz, 1H), 4.53 (t, J=11.2 Hz, 1H), 4.06 (dd, J=4.4, 11.2 Hz, 1H), 3.69 (br s, 2H), 3.3-3.3 (m, 2H), 2.72 (s, 6H). LC-MS (ESI+): 608.1 (M+H⁺).

Compound 156: ¹H NMR (DMSO-d6, 400 MHz) δ 8.23 (br s, 1H), 7.8-7.9 (m, 2H), 7.74 (d, J=8.7 Hz, 2H), 7.5-7.6 (m, 2H), 7.4-7.5 (m, 2H), 7.3-7.4 (m, 2H), 7.2-7.3 (m, 1H), 7.2-7.2 (m, 2H), 5.10 (dd, J=4.3, 11.2 Hz, 1H), 4.53 (t, J=11.2 Hz, 1H), 4.06 (dd, J=4.3, 11.2, 1H), 3.69 (br s, 2H), 3.3-3.3 (m, 2H), 2.72 (s, 6H). LC-MS (ESI+): 608.1 (M+H⁺).

### Synthesis of (S,E)-3-(4-chlorophenyl)-N′-((4-chlorophenyl)sulfonyl)-N-(2-((4-methylpiperazin-1-yl)sulfonyl)ethyl)-4-phenyl-4,5-dihydro-1H-pyrazole-1-carboximidamide (Compound 157) & (R,E)-3-(4-chlorophenyl)-N′-((4-chlorophenyl)sulfonyl)-N-(2-((4-methylpiperazin-1-yl)sulfonyl)ethyl)-4-phenyl-4,5-dihydro-1H-pyrazole-1-carboximidamide (Compound 158)

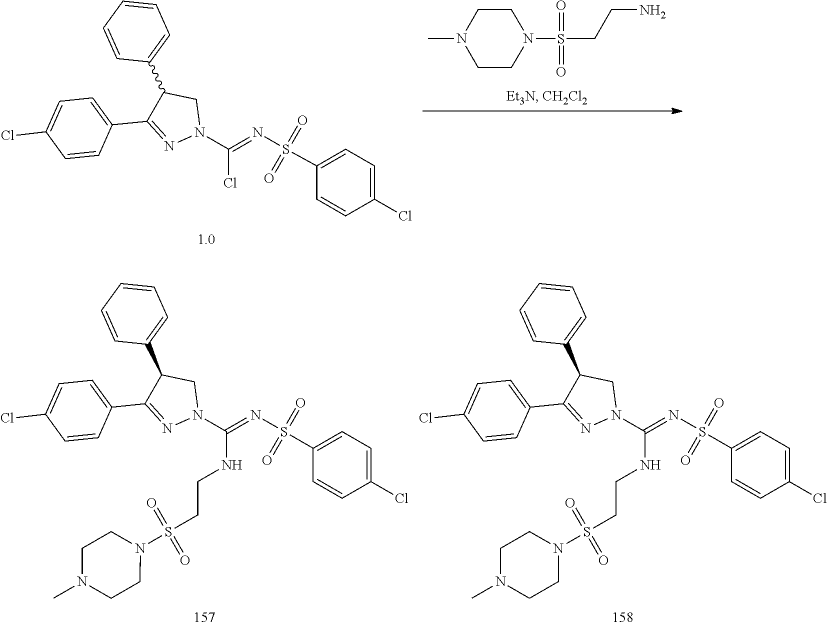 [SMILES: O=S(=O)(/N=C(\Cl)N1CC(c2ccccc2)C(c2ccc(Cl)cc2)=N1)c1ccc(Cl)cc1 ; CN1CCN(S(=O)(=O)CCN)CC1 ; CN1CCN(S(=O)(=O)CCN/C(=N\S(=O)(=O)c2ccc(Cl)cc2)N2C[C@H](c3ccccc3)C(c3ccc(Cl)cc3)=N2)CC1 ; CN1CCN(S(=O)(=O)CCN/C(=N\S(=O)(=O)c2ccc(Cl)cc2)N2C[C@H](c3ccccc3)C(c3ccc(Cl)cc3)=N2)CC1]

### Synthesis of Compounds 157 and 158

To a solution of 2-((4-methylpiperazin-1-yl)sulfonyl)ethan-1-amine (116 mg, 0.56 mmol, 1.1 eq.) and triethylamine (0.21 mL, 1.53 mmol, 3.0 eq.) in dichloromethane (5 mL) at rt was added (E)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0). After stirring at rt for 18 h, the reaction was diluted with dichloromethane (25 mL) and washed with aqueous HCl (2N, 20 mL). The organic layer was dried over magnesium sulfate, filtered, and concentrated in vacuo. The residue was purified by flash chromatography on silica eluting with methanol/dichloromethane mixtures (0/100 to 10/90, v/v) to give a colorless oil, which was further subjected to SFC separation conditions, obtaining the following enantiomers: Compound 157: (75.3 mg, 0.11 mmol, 22% yield, 98% purity, 99% ee, OR: −79.3) and Compound 158: (72.3 mg, 0.11 mol, 21% yield, 98% purity, 99% ee, OR: +112.6).

Method of SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO₂ (40%)—iPrOH+0.1% diethylamine, at 35° C., BPR 100 bar. Acquisition frequency was set to 220 nm for the DAD detector.

Compound 157: ¹H NMR (CDCl₃, 400 MHz) δ 7.77 (d, J=8.5 Hz, 2H), 7.46 (d, J=8.6 Hz, 2H), 7.34 (d, J=8.6 Hz, 3H), 7.2-7.3 (m, 5H), 7.0-7.1 (m, 2H), 4.66 (dd, J=5.6, 11.4 Hz, 1H), 4.45 (t, J=11.6 Hz, 1H), 4.0-4.1 (m, 2H), 3.98 (dd, J=5.7, 11.7 Hz, 1H), 3.7-3.9 (m, 4H), 3.41 (br s, 4H), 3.0-3.1 (m, 2H), 2.76 (d, J=4.3 Hz, 3H). LC-MS (ESI+): 663.1 (M+H⁺).

Compound 158: ¹H NMR (CDCl₃, 400 MHz) δ 7.85 (d, J=8.4 Hz, 2H), 7.54 (brd, J=8.4 Hz, 2H), 7.42 (d, J=8.4 Hz, 3H), 7.3-7.3 (m, 5H), 7.12 (br d, J=6.9 Hz, 2H), 4.74 (br dd, J=5.2, 10.8 Hz, 1H), 4.53 (br t, J=11.3 Hz, 1H), 4.1-4.2 (m, 2H), 4.0-4.1 (m, 1H), 3.8-4.0 (m, 4H), 3.49 (br d, J=7.5 Hz, 4H), 3.13 (br s, 2H), 2.84 (br s, 3H). LC-MS (ESI+): 663.1 (M+H⁺).

### Synthesis of (S,E)-3-(4-chlorophenyl)-N′-((4-chlorophenyl)sulfonyl)-4-phenyl-N-(2-(piperazin-1-ylsulfonyl)ethyl)-4,5-dihydro-1H-pyrazole-1-carboximidamide (Compound 159) & (S,E)-3-(4-chlorophenyl)-N′-((4-chlorophenyl)sulfonyl)-4-phenyl-N-(2-(piperazin-1-ylsulfonyl)ethyl)-4,5-dihydro-1H-pyrazole-1-carboximidamide (Compound 160)

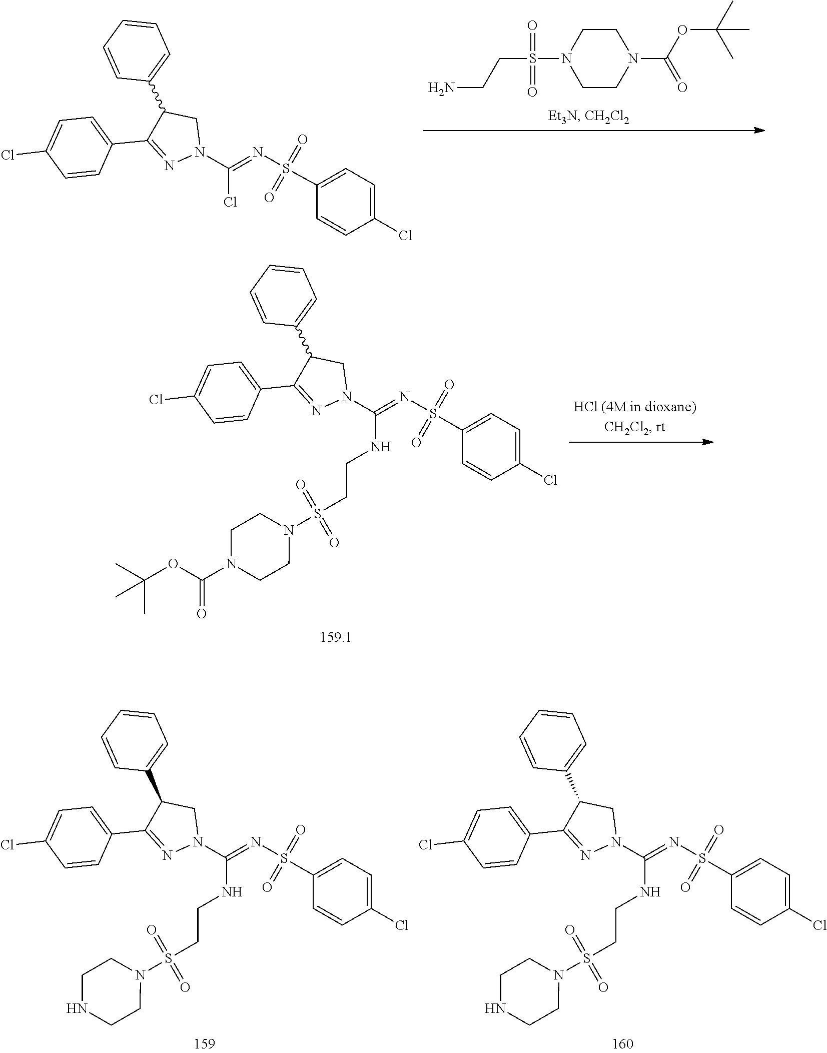 [SMILES: O=S(=O)(/N=C(\Cl)N1CC(c2ccccc2)C(c2ccc(Cl)cc2)=N1)c1ccc(Cl)cc1 ; CC(C)(C)OC(=O)N1CCN(S(=O)(=O)CCN)CC1 ; CC(C)(C)OC(=O)N1CCN(S(=O)(=O)CCN/C(=N\S(=O)(=O)c2ccc(Cl)cc2)N2CC(c3ccccc3)C(c3ccc(Cl)cc3)=N2)CC1 ; O=S(=O)(/N=C(\NCCS(=O)(=O)N1CCNCC1)N1C[C@H](c2ccccc2)C(c2ccc(Cl)cc2)=N1)c1ccc(Cl)cc1 ; O=S(=O)(/N=C(\NCCS(=O)(=O)N1CCNCC1)N1C[C@@H](c2ccccc2)C(c2ccc(Cl)cc2)=N1)c1ccc(Cl)cc1]

### Synthesis of tert-butyl (E)-4-((2-(3-(4-chlorophenyl)-N′-((4-chlorophenyl)sulfonyl)-4-phenyl-4,5-dihydro-1H-pyrazole-1-carboximidamido)ethyl)sulfonyl)piperazine-1-carboxylate (159.1)

To a stirred solution of tert-butyl 4-((2-aminoethyl)sulfonyl)piperazine-1-carboxylate (164 mg, 0.56 mmol, 1.1 eq.) and triethylamine (0.21 mL, 1.52 mmol, 3.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0). After stirring at rt for 18 h, the reaction was diluted with dichloromethane (25 mL) and washed with aqueous HCl (2N, 20 mL). The organic layer was dried over magnesium sulfate, filtered, and concentrated in vacuo. The residue was purified by flash chromatography on silica eluting with methanol/dichloromethane mixtures (0/100 to 10/90, v/v) to give a colorless oil (0.33 g, 0.44 mmol, 86% yield). LC-MS (ESI+): 749.100 (M+H⁺); HPLC RT: 2.08 min (Method: TACC21-6).

### Synthesis of Compounds 159 and 160

tert-butyl (E)-4-((2-(3-(4-chlorophenyl)-N′-((4-chlorophenyl)sulfonyl)-4-phenyl-4,5-dihydro-1H-pyrazole-1-carboximidamido)ethyl)sulfonyl)piperazine-1-carboxylate (330 mg, 0.44 mmol, 159.1) in dichloromethane (9 mL) was treated with a solution of hydrogen chloride in dioxane (4M, 0.55 mL, 2.20 mmol, 5.0 eq.). After stirring at rt for 18 h, the mixture was concentrated to dryness and chromatographed on silica eluting with dichloromethane/methanol mixtures (0/100 to 10/90, v/v) to afford a colorless oil which was submitted to SFC purification (SOP2 i-amylose-A, EtOH and Amylose-1, MeOH) to afford separate enantiomers: Compound 159 (57.8 mg, 0.09 mmol, 20% yield, 97% purity, 99% ee, OR: −28.3) and Compound 160 (57.1 mg, 0.09 mol, 20% yield, 99% purity, 99% ee, OR: +79.0).

Method of SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO₂ (40%)—EtOH+0.1% diethylamine, at 35° C., BPR 100 bar. Acquisition frequency was set to 220 nm for the DAD detector.

Compound 159: ¹H NMR (DMSO-d₆, 400 MHz) δ 8.21 (brt, J=5.1 Hz, 1H), 7.8 (m, 2H), 7.7-7.8 (m, 2H), 7.5-7.6 (m, 2H), 7.4-7.5 (m, 2H), 7.3-7.4 (m, 2H), 7.2-7.3 (m, 3H), 5.10 (dd, J=4.4, 11.2 Hz, 1H), 4.54 (t, J=11.2 Hz, 1H), 4.08 (dd, J=4.3, 11.1 Hz, 1H), 3.6-3.7 (m, 2H), 3.24 (br d, J=6.9 Hz, 2H), 3.0 (m, 4H), 2.7 (m, 4H). LC-MS (ESI+): 649.1 (M+H⁺).

Compound 160: ¹H NMR (DMSO-d₆, 400 MHz) δ 8.21 (br s, 1H), 7.8-7.8 (m, 1H), 7.8-7.8 (m, 1H), 7.74 (d, J=8.7 Hz, 2H), 7.5-7.6 (m, 2H), 7.4-7.5 (m, 2H), 7.3-7.4 (m, 2H), 7.2-7.3 (m, 3H), 5.10 (dd, J=4.3, 11.2 Hz, 1H), 4.54 (t, J=11.2 Hz, 1H), 4.08 (dd, J=4.4, 11.2 Hz, 1H), 3.68 (br s, 2H), 3.2-3.3 (m, 3H), 3.0 (m, 4H), 2.7 (m, 4H). LC-MS (ESI+): 649.1 (M+H⁺).

### Synthesis of (S,E)-N′-((4-chlorophenyl)sulfonyl)-N-(2-(N,N-dimethylsulfamoyl)ethyl)-3-(4-fluorophenyl)-4-phenyl-4,5-dihydro-1H-pyrazole-1-carboximidamide (Compound 161) & (R,E)-N′-((4-chlorophenyl)sulfonyl)-N-(2-(N,N-dimethylsulfamoyl)ethyl)-3-(4-fluorophenyl)-4-phenyl-4,5-dihydro-1H-pyrazole-1-carboximidamide (Compound 162)

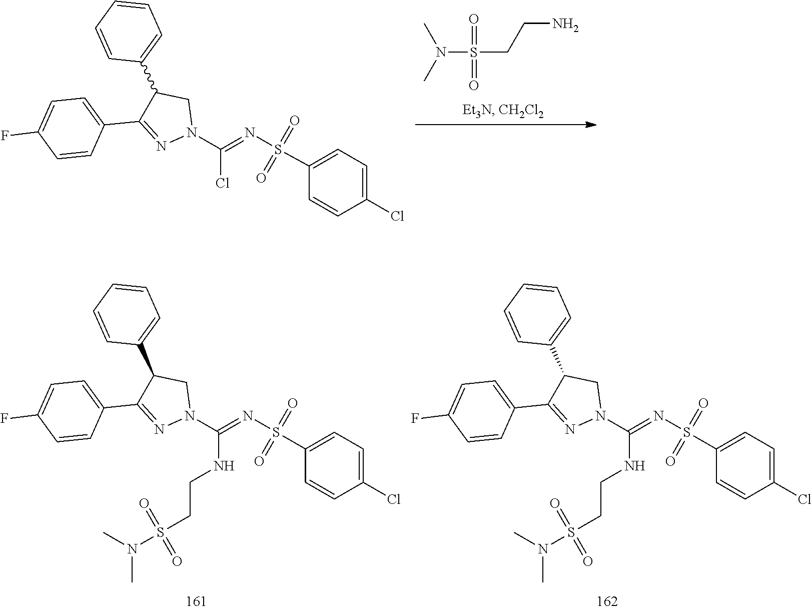

### Synthesis of Compounds 161 and 162

To a solution of 2-amino-N,N-dimethylethane-1-sulfonamide (88 mg, 0.58 mmol, 1.1 eq.) and triethylamine (0.22 mL, 1.57 mmol, 3.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (250 mg, 0.53 mmol, 1.0). After stirring at rt for 18 h, the reaction was diluted with dichloromethane (25 mL) and washed with aqueous HCl (2N, 20 mL). The organic layer was dried over magnesium sulfate, filtered, and concentrated in vacuo. The residue was purified by flash chromatography on silica eluting with methanol/dichloromethane mixtures (0/100 to 10/90, v/v) to give a colorless oil, which was further subjected to SFC separation, giving the following 2 enantiomers: Compound 161 (44.0 mg, 0.075 mmol, 14% yield, 99% purity, 99% ee, OR: −113.7) and Compound 162 (43.5 mg, 0.074 mol, 14% yield, 97% purity, 99% ee, OR: +121.4).

Method of SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO₂ (40%)—iPrOH+0.1% diethylamine, at 35° C., BPR 100 bar. Acquisition frequency was set to 220 nm for the DAD detector.

Compound 161: ¹H NMR (DMSO-d6, 400 MHz) δ 8.20 (brt, J=5.6 Hz, 1H), 7.83 (d, J=8.6 Hz, 3H), 7.8 (m, 1H), 7.5-7.6 (m, 2H), 7.33 (d, J=7.6 Hz, 2H), 7.2-7.3 (m, 5H), 5.10 (dd, J=4.3, 11.1 Hz, 1H), 4.52 (t, J=11.2 Hz, 1H), 4.06 (dd, J=4.3, 11.2 Hz, 1H), 3.6-3.7 (m, 2H), 3.3 (m, 2H), 2.72 (s, 6H). LC-MS (ESI+): 592.0 (M+H⁺).

Compound 162: ¹H NMR (DMSO-d6, 400 MHz) δ 8.20 (brt, J=5.6 Hz, 1H), 7.8-7.9 (m, 4H), 7.5-7.6 (m, 2H), 7.3-7.4 (m, 2H), 7.2-7.3 (m, 5H), 5.10 (dd, J=4.3, 11.1 Hz, 1H), 4.52 (t, J=11.2 Hz, 1H), 4.07 (dd, J=4.3, 11.2 Hz, 1H), 3.6-3.7 (m, 2H), 3.3 (m, 2H), 2.72 (s, 6H). LC-MS (ESI+): 591.5 (M+H⁺).

### Synthesis of (S,E)-N′-((4-chlorophenyl)sulfonyl)-3-(4-fluorophenyl)-N-(2-((4-methylpiperazin-1-yl)sulfonyl)ethyl)-4-phenyl-4,5-dihydro-1H-pyrazole-1-carboximidamide (Compound 163) & (R,E)-N′-((4-chlorophenyl)sulfonyl)-3-(4-fluorophenyl)-N-(2-((4-methylpiperazin-1-yl)sulfonyl)ethyl)-4-phenyl-4,5-dihydro-1H-pyrazole-1-carboximidamide (Compound 164)

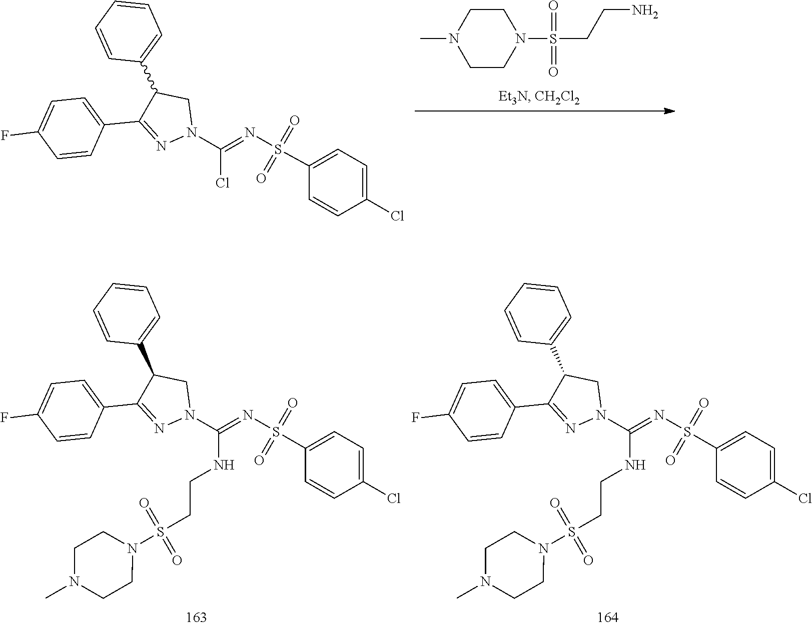 [SMILES: O=S(=O)(/N=C(\Cl)N1CC(c2ccccc2)C(c2ccc(F)cc2)=N1)c1ccc(Cl)cc1 ; CN1CCN(S(=O)(=O)CCN)CC1 ; CN1CCN(S(=O)(=O)CCN/C(=N\S(=O)(=O)c2ccc(Cl)cc2)N2C[C@H](c3ccccc3)C(c3ccc(F)cc3)=N2)CC1 ; CN1CCN(S(=O)(=O)CCN/C(=N\S(=O)(=O)c2ccc(Cl)cc2)N2C[C@@H](c3ccccc3)C(c3ccc(F)cc3)=N2)CC1]

To a solution of 2-((4-methylpiperazin-1-yl)sulfonyl)ethan-1-amine (120 mg, 0.58 mmol, 1.1 eq.) and triethylamine (0.22 mL, 1.58 mmol, 3.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (250 mg, 0.53 mmol, 45.1). After stirring at rt for 18 h, the reaction was diluted with dichloromethane (25 mL) and washed with aqueous HCl (2N, 20 mL). The organic layer was dried over magnesium sulfate, filtered, and concentrated in vacuo. The residue was purified by flash chromatography on silica eluting with methanol/dichloromethane mixtures (0/100 to 10/90, v/v) to give a colorless oil, which was further subjected to SFC separation, giving the following 2 enantiomers: Compound 163 (79.6 mg, 0.12 mmol, 24% yield, 99% purity, 99% ee, OR: −103.0) and Compound 164 (93.2 mg, 0.14 mol, 27% yield, 99% purity, 99% ee, OR: +109.2).

Method of SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO₂ (40%)—iPrOH+0.1% diethylamine, at 35° C., BPR 100 bar. Acquisition frequency was set to 220 nm for the DAD detector.

Compound 163: ¹H NMR (DMSO-d₆, 400 MHz) δ 8.18 (brt, J=5.5 Hz, 1H), 7.8-7.9 (m, 4H), 7.5-7.6 (m, 2H), 7.3-7.4 (m, 2H), 7.2-7.3 (m, 5H), 5.11 (dd, J=4.3, 11.1 Hz, 1H), 4.53 (t, J=11.2 Hz, 1H), 4.09 (dd, J=4.3, 11.2 Hz, 1H), 3.6-3.7 (m, 2H), 3.3-3.3 (m, 2H), 3.1 (m, 4H), 2.33 (br s, 4H), 2.19 (s, 3H). LC-MS (ESI+): 647.1 (M+H⁺).

Compound 164: ¹H NMR (DMSO-d₆, 400 MHz) δ 8.18 (brt, J=5.4 Hz, 1H), 7.8 (m, 4H), 7.5-7.6 (m, 1H), 7.5 (m, 1H), 7.3-7.4 (m, 2H), 7.2-7.3 (m, 5H), 5.11 (dd, J=4.3, 11.1 Hz, 1H), 4.53 (t, J=11.2 Hz, 1H), 4.09 (dd, J=4.2, 11.1 Hz, 1H), 3.6-3.7 (m, 2H), 3.3 (m, 2H), 3.1 (m, 4H), 2.33 (br s, 4H), 2.19 (s, 3H). LC-MS (ESI+): 647.2 (M+H⁺).

### Synthesis of (S,E)-3-(4-fluorophenyl)-N′-((4-fluorophenyl)sulfonyl)-N-(2-((4-methylpiperazin-1-yl)sulfonyl)ethyl)-4-phenyl-4,5-dihydro-1H-pyrazole-1-carboximidamide (Compound 165) & (R,E)-3-(4-fluorophenyl)-N′-((4-fluorophenyl)sulfonyl)-N-(2-((4-methylpiperazin-1-yl)sulfonyl)ethyl)-4-phenyl-4,5-dihydro-1H-pyrazole-1-carboximidamide (Compound 166)

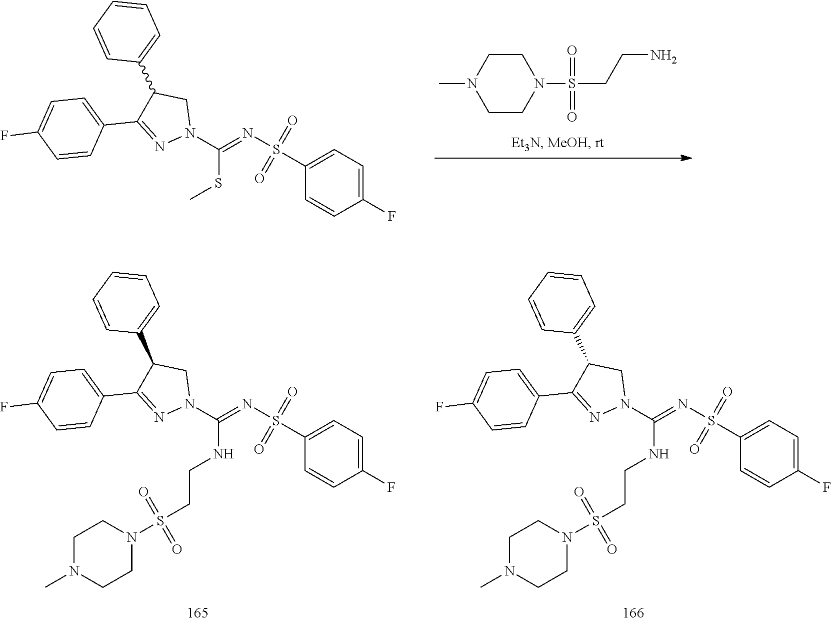 [SMILES: CS/C(=N\S(=O)(=O)c1ccc(F)cc1)N1CC(c2ccccc2)C(c2ccc(F)cc2)=N1 ; CN1CCN(S(=O)(=O)CCN)CC1 ; CN1CCN(S(=O)(=O)CCN/C(=N\S(=O)(=O)c2ccc(F)cc2)N2C[C@H](c3ccccc3)C(c3ccc(F)cc3)=N2)CC1 ; CN1CCN(S(=O)(=O)CCN/C(=N\S(=O)(=O)c2ccc(F)cc2)N2C[C@@H](c3ccccc3)C(c3ccc(F)cc3)=N2)CC1]

### Synthesis of Compounds 165 and 166

2-((4-methylpiperazin-1-yl)sulfonyl)ethan-1-amine (132 mg, 0.64 mmol, 2.0 eq.) was added to a stirring solution of methyl 3-(4-fluorophenyl)-N-((4-fluorophenyl)sulfonyl)-4-phenyl-4,5-dihydro-1H-pyrazole-1-carbimidothioate (150 mg, 0.32 mmol, 51.1) and triethylamine (0.13 mL, 0.95 mmol, 3.0 eq.) in methanol (1.2 mL). After stirring at rt for 18 h, the reaction was diluted with dichloromethane (25 mL) and washed with water (20 mL). The organic layer was dried over magnesium sulfate, filtered, and concentrated in vacuo. The residue purified by flash chromatography on silica eluting with ethyl acetate/heptane mixtures (0/100 to 50/50, v/v) to give a yellowish solid, which was further subjected to SFC separation, giving the following 2 enantiomers: Compound 165 (82.0 mg, 0.13 mmol, 41% yield, 96% purity, 99% ee, OR: −83.2) and Compound 166 (86.0 mg, 0.14 mol, 43% yield, 99% purity, 99% ee, OR: +91.1).

Method of SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO₂ (40%)—EtOH+0.1% diethylamine, at 35° C., BPR 100 bar. Acquisition frequency was set to 220 nm for the DAD detector.

Compound 165: ¹H NMR (DMSO-d₆, 400 MHz) δ 8.16 (br s, 1H), 7.8-7.9 (m, 2H), 7.8 (m, 2H), 7.2-7.4 (m, 9H), 5.10 (dd, J=4.3, 11.2 Hz, 1H), 4.53 (t, J=11.2 Hz, 1H), 4.09 (dd, J=4.3, 11.2 Hz, 1H), 3.6-3.7 (m, 2H), 3.30 (br d, J=1.8 Hz, 2H), 3.0-3.1 (m, 4H), 2.3-2.4 (m, 4H), 2.18 (s, 3H). LC-MS (ESI+): 631.1 (M+H⁺).

Compound 166: ¹H NMR (DMSO-d₆, 400 MHz) δ 8.16 (br s, 1H), 7.9-7.9 (m, 2H), 7.80 (dd, J=5.6, 8.8 Hz, 2H), 7.2-7.4 (m, 9H), 5.10 (dd, J=4.3, 11.1 Hz, 1H), 4.53 (t, J=11.2 Hz, 1H), 4.09 (dd, J=4.2, 11.1 Hz, 1H), 3.68 (br s, 2H), 3.30 (br d, J=2.0 Hz, 2H), 3.1 (m, 4H), 2.3-2.4 (m, 4H), 2.18 (s, 3H). LC-MS (ESI+): 631.1 (M+H⁺).

### Synthesis of (S,E)-N-(2-(N,N-dimethylsulfamoyl)ethyl)-3-(4-fluorophenyl)-N′-((4-fluorophenyl)sulfonyl)-4-phenyl-4,5-dihydro-1H-pyrazole-1-carboximidamide (Compound 167) & (R,E)-N-(2-(N,N-dimethylsulfamoyl)ethyl)-3-(4-fluorophenyl)-N′-((4-fluorophenyl)sulfonyl)-4-phenyl-4,5-dihydro-1H-pyrazole-1-carboximidamide (Compound 168)

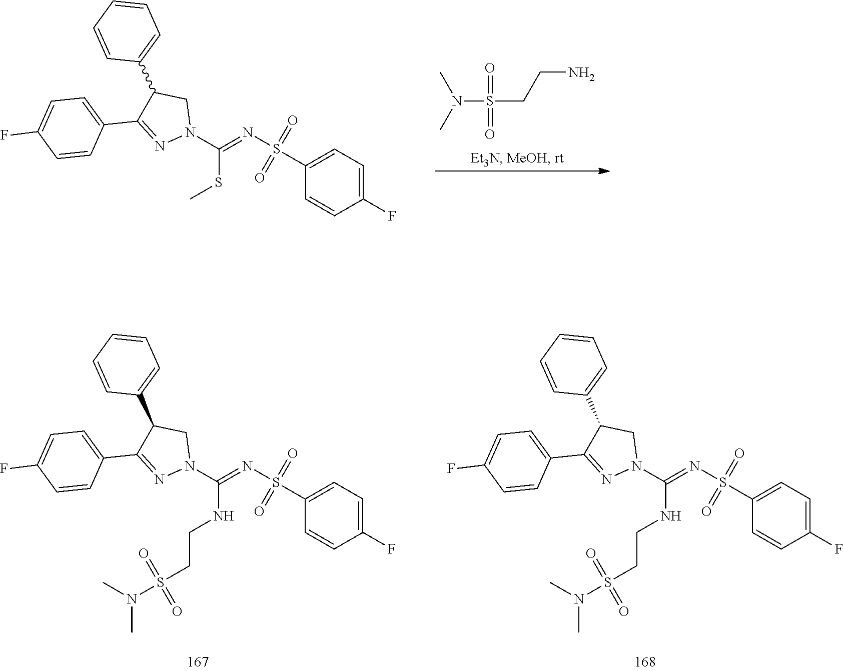

2-amino-N,N-dimethylethane-1-sulfonamide (97 mg, 0.64 mmol, 2.0 eq.) was added to a stirred solution of methyl 3-(4-fluorophenyl)-N-((4-fluorophenyl)sulfonyl)-4-phenyl-4,5-dihydro-1H-pyrazole-1-carbimidothioate (150 mg, 0.32 mmol, 51.1) and triethylamine (0.1 mL, 0.95 mmol, 3.0 eq.) in methanol (1.2 mL). After stirring at rt for 18 h, the reaction was diluted with dichloromethane (25 mL) and washed with water (20 mL). The organic layer was dried over magnesium sulfate, filtered, and concentrated in vacuo. The residue was purified by flash chromatography on silica eluting with ethyl acetate/heptane mixtures (0/100 to 50/50, v/v) to give a yellowish solid, which was further subjected to SFC separation, giving the following 2 enantiomers: Compound 167 (41.5 mg, 0.07 mmol, 23% yield, 99% purity, 99% ee, OR: −90.9) and Compound 168 (36.6 mg, 0.06 mol, 20% yield, 99% purity, 99% ee, OR: +101.9).

Method of SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO₂ (40%)—MeOH+0.1% diethylamine, at 35° C., BPR 100 bar. Acquisition frequency was set to 220 nm for the DAD detector.

Compound 167: ¹H NMR (DMSO-d₆, 400 MHz) δ 8.17 (brt, J=5.5 Hz, 1H), 7.8-7.9 (m, 2H), 7.8 (m, 2H), 7.2-7.4 (m, 9H), 5.09 (dd, J=4.3, 11.1 Hz, 1H), 4.52 (t, J=11.2 Hz, 1H), 4.07 (dd, J=4.3, 11.1 Hz, 1H), 3.6-3.7 (m, 2H), 3.3 (m, 2H), 2.72 (s, 6H). LC-MS (ESI+): 576.0 (M+H⁺).

Compound 168: ¹H NMR (DMSO-d₆, 400 MHz) δ 8.17 (brt, J=5.7 Hz, 1H), 7.8-7.9 (m, 2H), 7.8 (m, 2H), 7.2-7.4 (m, 9H), 5.09 (dd, J=4.3, 11.1 Hz, 1H), 4.52 (t, J=11.2 Hz, 1H), 4.07 (dd, J=4.3, 11.2 Hz, 1H), 3.7 (m, 2H), 3.3 (m, 2H), 2.72 (s, 6H). LC-MS (ESI+): 576.0 (M+H⁺).

### Synthesis of (S,E)-3-(4-chlorophenyl)-N′-((4-chlorophenyl)sulfonyl)-N-(2-(N-methylsulfamoyl)ethyl)-4-phenyl-4,5-dihydro-1H-pyrazole-1-carboximidamide (Compound 169) & (R,E)-3-(4-chlorophenyl)-N′-((4-chlorophenyl)sulfonyl)-N-(2-(N-methylsulfamoyl)ethyl)-4-phenyl-4,5-dihydro-1H-pyrazole-1-carboximidamide (Compound 170)

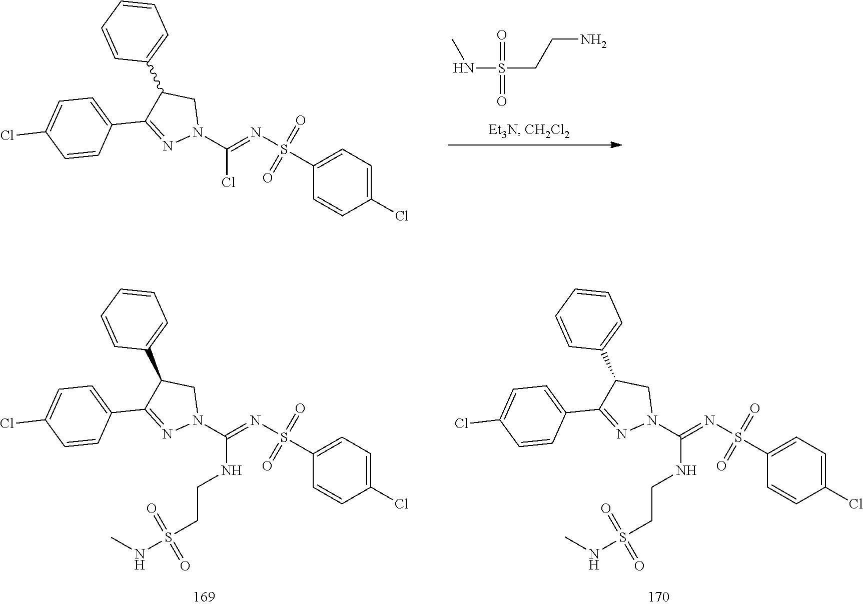

### Synthesis of Compound 168 and 170

To a solution of 2-amino-N-methylethane-1-sulfonamide (105 mg, 0.76 mmol, 1.1 eq.) and triethylamine (0.21 mL, 1.53 mmol, 3.0 eq.) in dichloromethane (5 mL) was added 4-Chloro-N-{chloro-[3-(4-chloro-phenyl)-4-phenyl-4,5-dihydro-pyrazol-1-yl]-methylene}-benzenesulfonamide (250 mg, 0.51 mmol, 1.0 eq.). After stirring at rt for 18 h, the reaction was diluted with dichloromethane (25 mL) and washed with aqueous ammonium chloride and water. The organic layer was dried over magnesium sulfate, filtered, and concentrated in vacuo. The residue was purified by flash chromatography on silica eluting with ethyl acetate/heptane mixtures (0/100 to 50/50, v/v) to give a colorless oil, which was further subjected to SFC separation, giving the following 2 enantiomers: Compound 169 (51.6 mg, 0.09 mmol, 17% yield, 99% purity, 99% ee, OR: −127.9) and Compound 170 (60.3 mg, 0.1 mol, 20% yield, 99% purity, 99% ee, OR: +104.2).

Method of SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 169: (51.6 mg, 0.09 mmol, 17% yield, 99% purity, 99% ee, OR: −127.9). ¹H NMR (DMSO-d₆, →00 MHz) δ 8.19 (br s, 1H), 7.8 (m, 2H), 7.74 (d, J=8.7 Hz, 2H), 7.5-7.6 (m, 2H), 7.46 (d, J=8.7 Hz, 2H), 7.3-7.4 (m, 2H), 7.2-7.3 (m, 3H), 7.08 (q, J=4.5 Hz, 1H), 5.10 (dd, J=4.3, 11.2 Hz, 1H), 4.53 (t, J=11.2 Hz, 1H), 4.06 (dd, J=4.3, 11.1 Hz, 1H), 3.66 (q, J=6.3 Hz, 2H), 3.3 (m, 5H). LC-MS (ESI+): 594.0 (M+H⁺).

Compound 170: (60.3 mg, 0.1 mol, 20% yield, 99% purity, 99% ee, OR: +104.2). ¹H NMR (DMSO-d₆, 400 MHz) δ 8.19 (br s, 1H), 7.82 (d, J=8.6 Hz, 2H), 7.74 (d, J=8.6 Hz, 2H), 7.54 (d, J=8.6 Hz, 2H), 7.46 (d, J=8.7 Hz, 2H), 7.3-7.4 (m, 2H), 7.2-7.3 (m, 3H), 7.08 (br s, 1H), 5.10 (dd, J=4.3, 11.2 Hz, 1H), 4.53 (t, J=11.2 Hz, 1H), 4.06 (dd, J=4.3, 11.1 Hz, 1H), 3.66 (br s, 2H), 3.2-3.3 (m, 5H). LC-MS (ESI+): 594.0 (M+H⁺).

### Synthesis of (S,E)-3-(4-chlorophenyl)-N′-((4-chlorophenyl)sulfonyl)-4-phenyl-N-(2-(pyrrolidin-1-ylsulfonyl)ethyl)-4,5-dihydro-1H-pyrazole-1-carboximidamide (Compound 171) & (R,E)-3-(4-chlorophenyl)-N′-((4-chlorophenyl)sulfonyl)-4-phenyl-N-(2-(pyrrolidin-1-ylsulfonyl)ethyl)-4,5-dihydro-1H-pyrazole-1-carboximidamide (Compound 172)

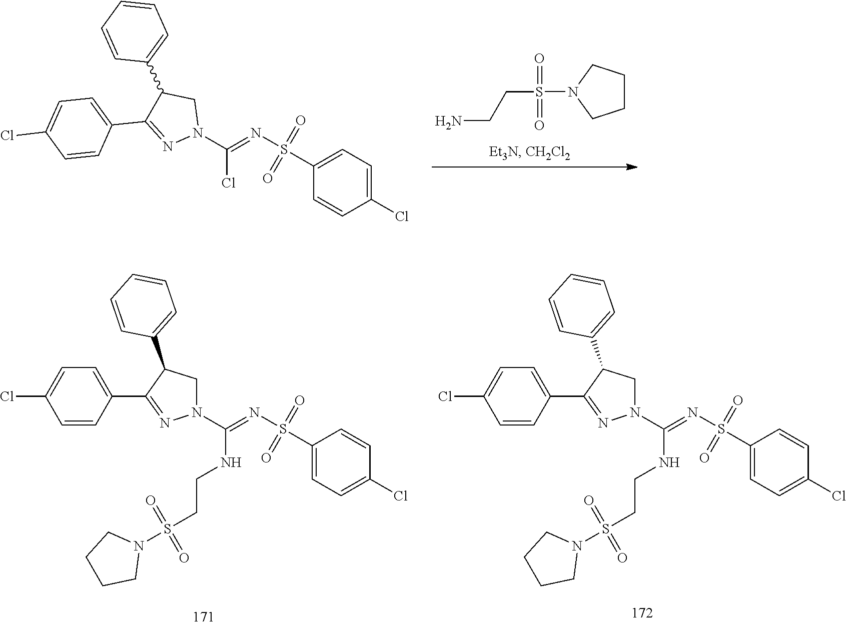 [SMILES: O=S(=O)(/N=C(\Cl)N1CC(c2ccccc2)C(c2ccc(Cl)cc2)=N1)c1ccc(Cl)cc1 ; NCCS(=O)(=O)N1CCCC1 ; O=S(=O)(/N=C(\NCCS(=O)(=O)N1CCCC1)N1C[C@H](c2ccccc2)C(c2ccc(Cl)cc2)=N1)c1ccc(Cl)cc1 ; O=S(=O)(/N=C(\NCCS(=O)(=O)N1CCCC1)N1C[C@@H](c2ccccc2)C(c2ccc(Cl)cc2)=N1)c1ccc(Cl)cc1]

### Synthesis of Compounds 171 and 172

To a solution of 2-(pyrrolidine-1-sulfonyl)-ethylamine (135 mg, 0.76 mmol, 1.5 eq.) and triethylamine (0.21 mL, 1.52 mmol, 3.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0 eq.). After stirring at rt for 18 h, the reaction was diluted with dichloromethane (20 mL) and washed with ammonium chloride (20 mL). The organic layer was dried over magnesium sulfate, filtered, and concentrated in vacuo. The residue was purified by flash chromatography on silica eluting with ethyl acetate/heptane mixtures (0/100 to 50/50, v/v) to give a colorless oil which was submitted to SFC purification: Compound 171: (41.6 mg, 0.065 mmol, 13% yield, 99% purity, 99% ee, OR: −106.4) and Compound 172 (5 mg, 99% ee, OR+105).

Method of SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO₂ (40%)—MeOH+0.1% diethylamine, at 35° C., BPR 100 bar. Acquisition frequency was set to 220 nm for the DAD detector.

Compound 171: (41.6 mg, 0.065 mmol, 13% yield, 99% purity, 99% ee, OR: −106.4). ¹H NMR (DMSO-d₆, 400 MHz) δ 8.2-8.3 (m, 1H), 7.8-7.9 (m, 2H), 7.75 (d, J=8.7 Hz, 2H), 7.5-7.6 (m, 2H), 7.46 (d, J=8.7 Hz, 2H), 7.3-7.4 (m, 2H), 7.2-7.3 (m, 3H), 5.10 (dd, J=4.3, 11.2 Hz, 1H), 4.53 (t, J=11.2 Hz, 1H), 4.07 (dd, J=4.2, 11.1 Hz, 1H), 3.6-3.7 (m, 2H), 3.3-3.3 (m, 2H), 3.17 (br t, J=6.6 Hz, 4H), 1.8 (m, 4H). LC-MS (ESI+): 634.1 (M+H⁺).

Compound 172: (5 mg, 99% purity, 99% ee, OR: +105). 1H NMR (DMSO-d₆, 400 MHz) δ 8.2-8.3 (m, 1H), 7.8-7.9 (m, 2H), 7.75 (d, J=8.7 Hz, 2H), 7.5-7.6 (m, 2H), 7.46 (d, J=8.7 Hz, 2H), 7.3-7.4 (m, 2H), 7.2-7.3 (m, 3H), 5.10 (dd, J=4.3, 11.2 Hz, 1H), 4.53 (t, J=11.2 Hz, 1H), 4.07 (dd, J=4.2, 11.1 Hz, 1H), 3.6-3.7 (m, 2H), 3.3-3.3 (m, 2H), 3.17 (br t, J=6.6 Hz, 4H), 1.8 (m, 4H). LC-MS (ESI+): 634.1 (M+H⁺).

### Synthesis of (S,E)-3-(4-chlorophenyl)-N′-((4-chlorophenyl)sulfonyl)-N-methyl-4-phenyl-N-(2-(sulfamoylamino)ethyl)-4,5-dihydro-1H-pyrazole-1-carboximidamide (Compound 173) & (R,E)-3-(4-chlorophenyl)-N′-((4-chlorophenyl)sulfonyl)-N-methyl-4-phenyl-N-(2-(sulfamoylamino)ethyl)-4,5-dihydro-1H-pyrazole-1-carboximidamide (Compound 174)

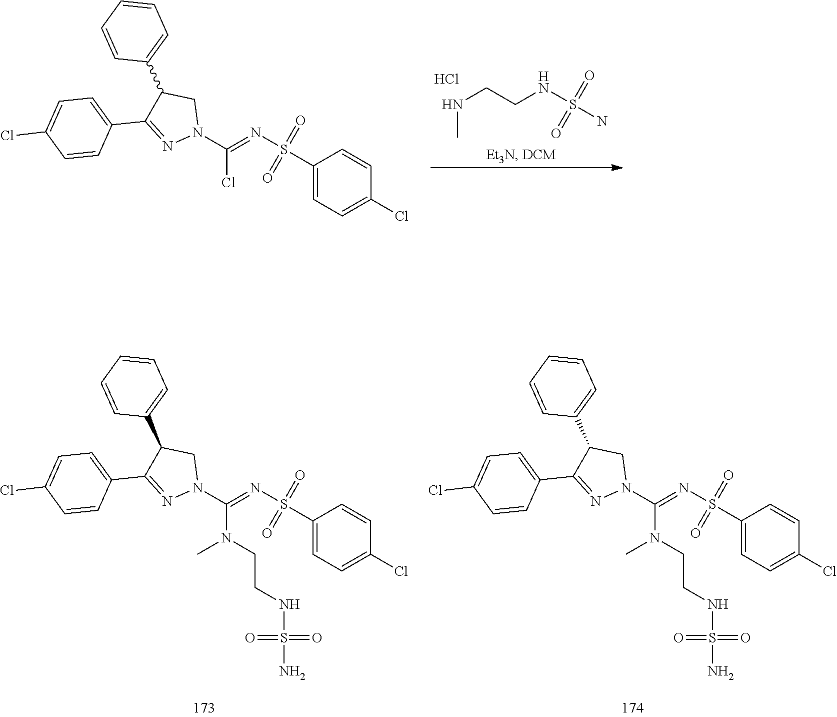

### Synthesis of Compounds 173 and 174

To a stirred solution of (2-(methylamino)ethyl)sulfamide hydrochloride (144 mg, 0.76 mmol, 1.5 eq.) and triethylamine (0.35 mL, 2.53 mmol, 5.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0 eq.). After stirring at rt for 18 h, solvents were removed under reduced pressure. The residue was chromatographed on silica eluting with ethyl acetate/heptane mixtures (0/100 to 80/20, v/v) to obtain a colorless oil, which was further subjected to SFC purification to resolve the enantiomers:

Method of the SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO2 (40%)—EtOH+0.1% diethylamine, at 35° C., BPR 100 bar. Acquisition frequency was set to 220 nm for the DAD detector.

Compound 173: (8.2 mg, 0.013 mmol, 3% yield, 99% purity, 99% ee, OR: −140.1). 1H NMR (MeOD-d₄, 400 MHz) δ 7.8-7.9 (m, 2H), 7.6 (m, 2H), 7.4-7.5 (m, 2H), 7.2-7.4 (m, 7H), 4.8 (m, 1H), 4.44 (t, J=11.3 Hz, 1H), 3.9-4.0 (m, 1H), 3.8-3.9 (m, 1H), 3.74 (dd, J=5.3, 11.2 Hz, 1H), 3.40 (t, J=5.8 Hz, 2H), 3.28 (s, 3H). LC-MS (ESI+): 609.1 (M+H⁺).

Compound 174: (8.6 mg, 0.013 mmol, 3% yield, 99% purity, 99% ee, OR: +90.9). 1H NMR (MeOD-d₄, 400 MHz) δ 7.8-7.9 (m, 2H), 7.59 (d, J=8.7 Hz, 2H), 7.45 (d, J=8.6 Hz, 2H), 7.2-7.4 (m, 7H), 4.83 (m, 1H), 4.44 (t, J=11.2 Hz, 1H), 3.9-4.0 (m, 1H), 3.8-3.9 (m, 1H), 3.74 (dd, J=5.3, 11.2 Hz, 1H), 3.40 (t, J=5.8 Hz, 2H), 3.28 (s, 3H). LC-MS (ESI+): 609.1 (M+H⁺).

### Synthesis of (S,E)-N′-((4-chlorophenyl)sulfonyl)-3-(4-fluorophenyl)-N-(2-(N-methylsulfamoyl)ethyl)-4-phenyl-4,5-dihydro-1H-pyrazole-1-carboximidamide (Compound 175) & (R,E)-N′-((4-chlorophenyl)sulfonyl)-3-(4-fluorophenyl)-N-(2-(N-methylsulfamoyl)ethyl)-4-phenyl-4,5-dihydro-1H-pyrazole-1-carboximidamide (Compound 176)

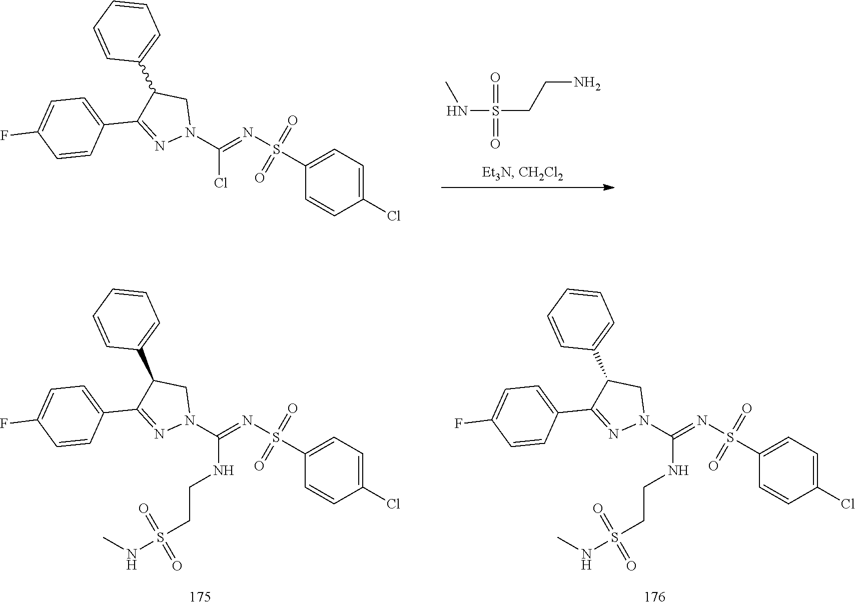

### Synthesis of Compound 175 and 176

To a solution of 2-amino-N-methylethane-1-sulfonamide (80 mg, 0.6 mmol, 1.1 eq.) and triethylamine (0.22 mL, 1.58 mmol, 3.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (250 mg, 0.53 mmol, 1.0 eq.). After stirring at rt for 18 h, the reaction was diluted with dichloromethane (25 mL) and washed with aqueous ammonium chloride (20 mL) and water (20 mL). The organic layer was dried over magnesium sulfate, filtered, and concentrated in vacuo. The residue was purified by flash chromatography on silica eluting with ethyl acetate/heptane mixtures (0/100 to 50/50, v/v) to give a colorless oil, which was further subjected to SFC separation conditions, obtaining the following 2 enantiomers: Compound 175 (7.0 mg, 0.012 mmol, 2% yield, 99% purity, 99% ee, OR: −100.0) and Compound 176 (8.0 mg, 0.014 mol, 3% yield, 96% purity, 99% ee, OR: +101.7).

Method of SFC: SFC purification was performed on a Jasco 4000 Parallel SFC System (4× Channels 3×UV+1×DAD) using an Phenomenex column (LuxAmylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 175: ¹H NMR (DMSO-d₆, 400 MHz) δ 8.04-8.30 (m, 1H), 7.72-7.87 (m, 4H), 7.53 (d, J=8.5 Hz, 2H), 7.30-7.37 (m, 2H), 7.19-7.29 (m, 5H), 7.09 (br s, 1H), 5.07 (br d, J=2.6 Hz, 1H), 4.42-4.57 (m, 1H), 4.05 (br dd, J=8.2, 1.5 Hz, 1H), 3.61-3.71 (m, 2H), 3.27 ppm (br s, 2H) 2.5 ppm (s overlapped with DMSO-d₆, 3H). LC-MS (ESI+): 578.1 (M+H⁺).

Compound 176: ¹H NMR (DMSO-d₆, 400 MHz) δ 8.17 (brd, J=2.9 Hz, 1H), 7.71-7.86 (m, 4H), 7.53 (d, J=8.6 Hz, 2H), 7.32 (d, J=7.5 Hz, 2H), 7.18-7.28 (m, 5H), 7.08 (br d, J=2.6 Hz, 1H), 5.09 (br dd, J=11.0, 3.7 Hz, 1H), 4.51 (br t, J=11.0 Hz, 1H), 4.05 (br dd, J=10.9, 3.7 Hz, 1H), 3.66 (br t, J=6.6 Hz, 2H), 3.24-3.28 ppm (m, 2H), 2.5 ppm (s overlapped with DMSO-d₆, 3H). LC-MS (ESI+): 578.1 (M+H⁺).

### Synthesis of (S)-3-(4-chlorophenyl)-N′-((4-fluorophenyl)sulfonyl)-4-phenyl-N-(2-(piperazin-1-ylsulfonyl)ethyl)-4,5-dihydro-1H-pyrazole-1-carboximidamide (Compound 177) & (R)-3-(4-chlorophenyl)-N′-((4-fluorophenyl)sulfonyl)-4-phenyl-N-(2-(piperazin-1-ylsulfonyl)ethyl)-4,5-dihydro-1H-pyrazole-1-carboximidamide (Compound 178)

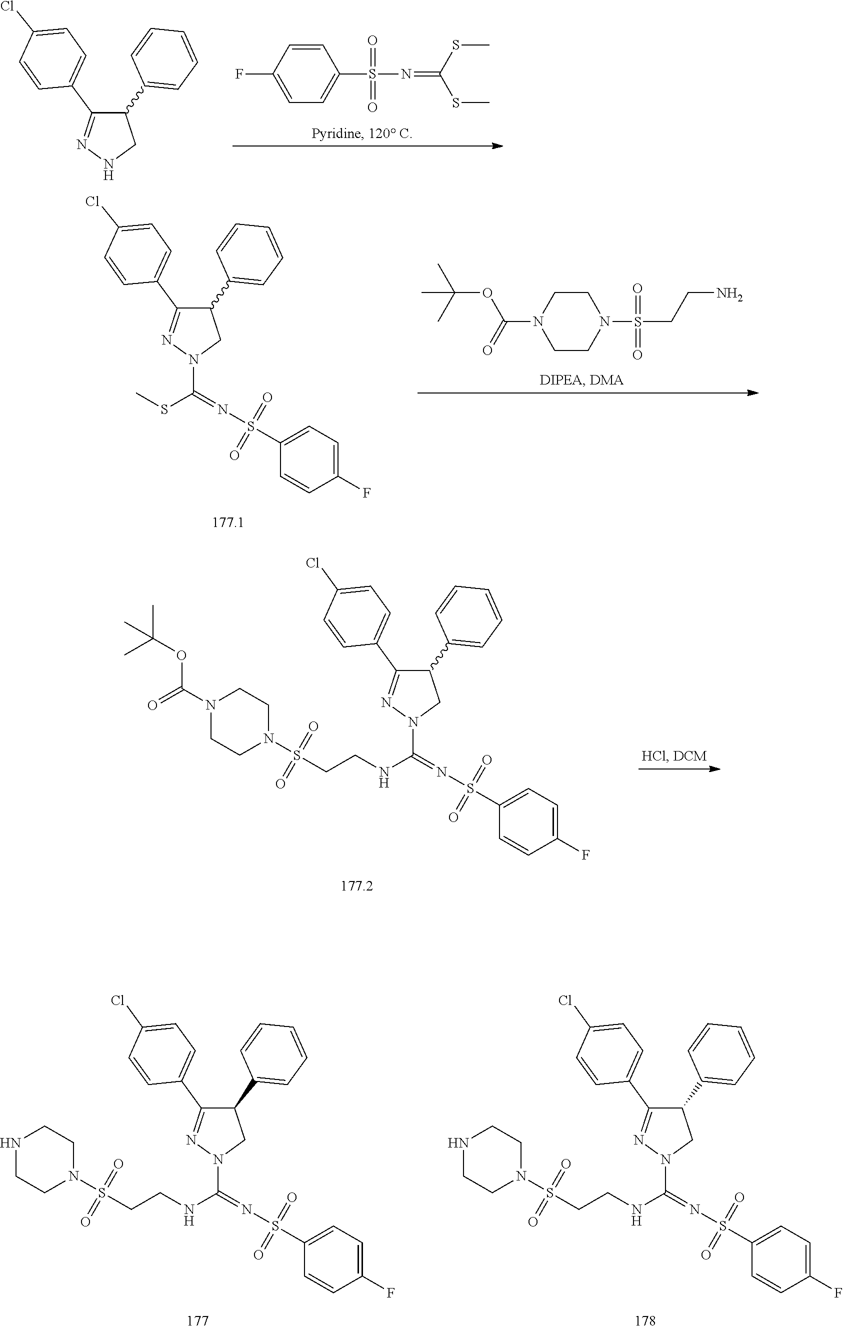

### Synthesis of 177.1

Dimethyl ((4-fluorophenyl)sulfonyl)carbonimidodithioate (4.0 g, 14.3 mmol, 1 eq.) and 3-(4-chlorophenyl)-4-phenyl-4,5-dihydro-1H-pyrazole (3.7 g, 14.3 mmol, 1 eq.) in pyridine (20 mL) were heated at 120° C. for 18 h. Upon completion, the reaction mixture was concentrated to dryness. The residue was purified by flash chromatography on silica eluting with ethyl acetate/heptane mixtures (0/100 to 40/60, v/v) to afford the expected product as a yellow foamy solid (2.8 g, 5.75 mmol, 40% yield). LC-MS (ESI+): 488.1 (M+H⁺).

### Synthesis of 177.2

To a stirred solution of tert-butyl 4-((2-aminoethyl)sulfonyl)piperazine-1-carboxylate (0.3 g, 1.02 mmol, 2.0 eq.) and N,N-diisopropylethylamine (0.31 mL, 1.79 mmol, 3.50 eq.) in N,N-dimethylacetamide (5 mL) was added methyl 3-(4-chlorophenyl)-N-((4-fluorophenyl)sulfonyl)-4-phenyl-4,5-dihydro-1H-pyrazole-1-carbimidothioate (0.25 g, 0.51 mmol, 177.1). After stirring at rt for 18 h, solvents were removed under reduced pressure and diluted with dichloromethane (20 mL). The organic solution was washed with hydrogen chloride 1 N solution (15 mL) and brine (20 mL). The organic extract was dried over magnesium sulfate, filtered, and concentrated in vacuo. The residue was chromatographed on silica eluting with methanol/dichloromethane mixtures (0/100 to 10/90, v/v) to give the expected compound as a colorless oil (0.28 g, 0.38 mmol, 75% yield). LC-MS (ESI+): 733.1 (M+H⁺).

### Synthesis of Compounds 177 and 178

Hydrogen chloride 4M in dioxane (0.47 mL, 1.88 mmol, 5.0 eq.) was added to a stirring solution of tert-butyl (E)-4-((2-(3-(4-chlorophenyl)-N′-((4-fluorophenyl)sulfonyl)-4-phenyl-4,5-dihydro-1H-pyrazole-1-carboximidamido)ethyl)sulfonyl)piperazine-1-carboxylate (0.28 mg, 0.38 mmol, 177.2) in dichloromethane (3 mL). After stirring at rt for 18 h, solvents were removed under reduced pressure. The residue was chromatographed on silica eluting with methanol/dichloromethane mixtures (0/100 to 10/90, v/v) to give a yellowish oil which was further subjected to SFC purification to resolve the two enantiomers: Compound 177 (40.9 mg, 0.065 mmol, 17% yield, 98% purity, 99% ee, OR: −54.4) and Compound 178 (37.5 mg, 0.06 mmol, 16% yield, 99% purity, 99% ee, OR: +99.2).

Method of SFC: SFC purification was performed on a Jasco 4000 Parallel SFC System (4× Channels 3×UV+1×DAD) using an Phenomenex column (LuxAmylose-1) 150 mm long x4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 177: ¹H NMR (DMSO-d₆, 400 MHz) δ 8.18 (br t, J=5.3 Hz, 1H), 7.83-7.91 (m, 2H), 7.75 (d, J=8.7 Hz, 2H), 7.46 (d, J=8.7 Hz, 2H), 7.17-7.38 (m, 7H), 5.10 (dd, J=11.2, 4.4 Hz, 1H), 4.54 (t, J=11.3 Hz, 1H), 4.09 (br dd, J=11.1, 4.3 Hz, 1H), 3.63-3.74 (m, 2H), 3.24-3.28 (m, 3H), 2.96-3.04 (m, 4H), 2.68-2.74 ppm (m, 4H). LC-MS (ESI+): 633.1 (M+H⁺).

Compound 178: ¹H NMR (DMSO-d₆, 400 MHz) δ 8.11 (br t, J=5.4 Hz, 1H), 7.77-7.85 (m, 2H), 7.69 (d, J=8.7 Hz, 2H), 7.40 (d, J=8.7 Hz, 2H), 7.13-7.30 (m, 7H), 5.03 (dd, J=11.2, 4.4 Hz, 1H), 4.47 (t, J=11.2 Hz, 1H), 4.02 (br dd, J=11.1, 4.3 Hz, 1H), 3.56-3.67 (m, 2H), 3.18-3.21 (m, 3H), 2.89-2.97 (m, 4H), 2.61-2.66 ppm (m, 4H). LC-MS (ESI+): 633.1 (M+H⁺).

### Synthesis of (S)-N′-((4-chlorophenyl)sulfonyl)-3-(4-fluorophenyl)-4-phenyl-N-(2-(piperazin-1-ylsulfonyl)ethyl)-4,5-dihydro-1H-pyrazole-1-carboximidamide (Compound 179) & (R)-N′-((4-chlorophenyl)sulfonyl)-3-(4-fluorophenyl)-4-phenyl-N-(2-(piperazin-1-ylsulfonyl)ethyl)-4,5-dihydro-1H-pyrazole-1-carboximidamide (Compound 180)

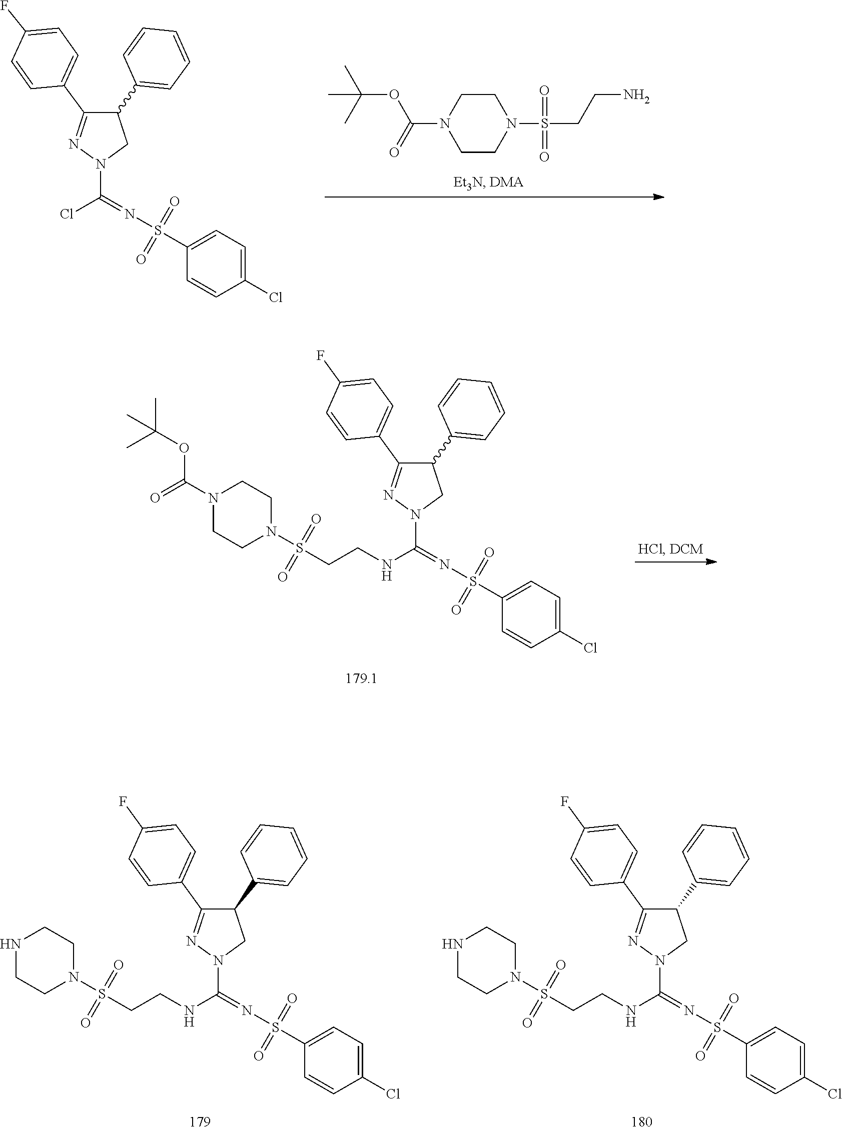 [SMILES: O=S(=O)(N=C(Cl)N1CC(c2ccccc2)C(c2ccc(F)cc2)=N1)c1ccc(Cl)cc1 ; CC(C)(C)OC(=O)N1CCN(S(=O)(=O)CCN)CC1 ; CC(C)(C)OC(=O)N1CCN(S(=O)(=O)CCNC(=NS(=O)(=O)c2ccc(Cl)cc2)N2CC(c3ccccc3)C(c3ccc(F)cc3)=N2)CC1 ; O=S(=O)(N=C(NCCS(=O)(=O)N1CCNCC1)N1C[C@H](c2ccccc2)C(c2ccc(F)cc2)=N1)c1ccc(Cl)cc1 ; O=S(=O)(N=C(NCCS(=O)(=O)N1CCNCC1)N1C[C@@H](c2ccccc2)C(c2ccc(F)cc2)=N1)c1ccc(Cl)cc1]

### Synthesis of 179.1

To a stirred solution of tert-butyl 4-((2-aminoethyl)sulfonyl)piperazine-1-carboxylate (0.17 g, 0.6 mmol, 1.1 eq.) and triethylamine (0.22 mL, 1.6 mmol, 3.0 eq.) in N,N-dimethylacetamide (5 mL) was added (Z)-N-((4-chlorophenyl)sulfonyl)-3-(4-fluorophenyl)-4-phenyl-4,5-dihydro-1H-pyrazole-1-carbimidoyl chloride (0.25 g, 0.53 mmol, 1.0 eq.). After stirring at rt for 18 h, solvents were removed under reduced pressure, diluted with dichloromethane (20 mL), and washed with hydrogen chloride 1 N solution (15 mL) and brine (20 mL). The organic extract was dried over magnesium sulfate, filtered, and concentrated in vacuo. The residue was chromatographed on silica eluting with methanol/dichloromethane mixtures (0/100 to 10/90, v/v) to give the expected compound as a colorless oil (0.23 g, 0.31 mmol, 60% yield). LC-MS (ESI+): 733.2 (M+H⁺).

### Synthesis of Compounds 179 and 180

Hydrogen chloride 4M in dioxane (0.4 mL, 1.6 mmol, 5.0 eq.) was added to a stirring solution of tert-butyl (E)-4-((2-(N′-((4-chlorophenyl)sulfonyl)-3-(4-fluorophenyl)-4-phenyl-4,5-dihydro-1H-pyrazole-1-carboximidamido)ethyl)sulfonyl)piperazine-1-carboxylate (0.23 mg, 0.31 mmol, 179.1) in dichloromethane (3 mL). After stirring at rt for 18 h, solvents were removed under reduced pressure and the crude was chromatographed on silica eluting with methanol/dichloromethane mixtures (0/100 to 10/90, v/v) to give a yellowish oil which was further subjected to SFC purification to resolve the two enantiomers: Compound 179 (13.3 mg, 0.02 mmol, 7% yield, 95% purity, 99% ee, OR: −107.8) and Compound 180 (25.4 mg, 0.04 mmol, 13% yield, 97% purity, 99% ee, OR: +107.4).

Method of SFC: SFC purification was performed on a Jasco 4000 Parallel SFC System (4× Channels 3×UV+1×DAD) using an Phenomenex column (Lux Amylose-A) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 179: ¹H NMR (DMSO-d₆, 400 MHz) δ 8.15-8.23 (m, 2H), 7.76-7.86 (m, 4H), 7.51-7.58 (m, 2H), 7.30-7.37 (m, 2H), 7.20-7.29 (m, 5H), 5.10 (dd, J=11.2, 4.2 Hz, 1H), 4.53 (t, J=11.2 Hz, 1H), 4.08 (br dd, J=11.2, 4.3 Hz, 1H), 3.65-3.70 (m, 2H), 3.26 (br d, J=1.6 Hz, 2H), 2.97-3.01 (m, 4H), 2.68-2.72 ppm (m, 4H). LC-MS (ESI+): 633.1 (M+H⁺).

Compound 180: ¹H NMR (DMSO-d₆, 400 MHz) δ 8.19 (brs, 1H), 7.76-7.84 (m, 4H), 7.51-7.58 (m, 2H), 7.30-7.36 (m, 2H), 7.21-7.29 (m, 6H), 5.10 (dd, J=11.1, 4.2 Hz, 1H), 4.52 (br t, J=11.1 Hz, 1H), 4.06 (br dd, J=11.1, 4.2 Hz, 1H), 3.64-3.73 (m, 2H), 3.28 (br s, 2H), 3.02-3.08 (m, 4H), 2.80 ppm (br d, J=4.4 Hz, 4H). LC-MS (ESI+): 633.1 (M+H⁺).

### Synthesis of (S)-3-(4-chlorophenyl)-4-phenyl-N-(2-(piperazin-1-ylsulfonyl)ethyl)-N′-((4-(trifluoromethyl)phenyl)sulfonyl)-4,5-dihydro-1H-pyrazole-1-carboximidamide (Compound 181) & (R)-3-(4-chlorophenyl)-4-phenyl-N-(2-(piperazin-1-ylsulfonyl)ethyl)-N′-((4-(trifluoromethyl)phenyl)sulfonyl)-4,5-dihydro-1H-pyrazole-1-carboximidamide (Compound 182)

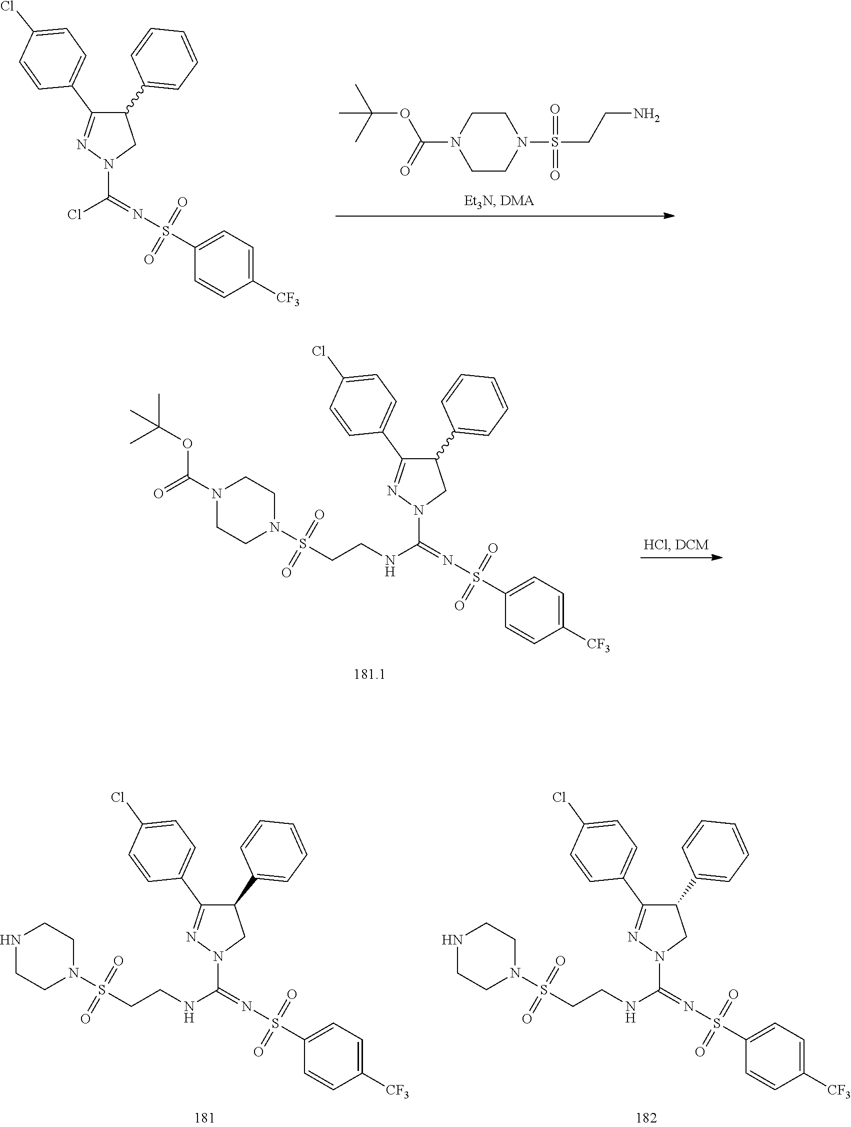 [SMILES: O=S(=O)(N=C(Cl)N1CC(c2ccccc2)C(c2ccc(Cl)cc2)=N1)c1ccc(C(F)(F)F)cc1 ; CC(C)(C)OC(=O)N1CCN(S(=O)(=O)CCN)CC1 ; CC(C)(C)OC(=O)N1CCN(S(=O)(=O)CCNC(=NS(=O)(=O)c2ccc(C(F)(F)F)cc2)N2CC(c3ccccc3)C(c3ccc(Cl)cc3)=N2)CC1 ; O=S(=O)(N=C(NCCS(=O)(=O)N1CCNCC1)N1C[C@H](c2ccccc2)C(c2ccc(Cl)cc2)=N1)c1ccc(C(F)(F)F)cc1 ; O=S(=O)(N=C(NCCS(=O)(=O)N1CCNCC1)N1C[C@@H](c2ccccc2)C(c2ccc(Cl)cc2)=N1)c1ccc(C(F)(F)F)cc1]

### Synthesis of 181.1

To a stirred solution of tert-butyl 4-((2-aminoethyl)sulfonyl)piperazine-1-carboxylate (0.15 g, 0.52 mmol, 1.1 eq.) and triethylamine (0.2 mL, 1.4 mmol, 3.0 eq.) in N,N-dimethylacetamide (5 mL) was added (Z)-3-(4-chlorophenyl)-4-phenyl-N-((4-(trifluoromethyl)phenyl)sulfonyl)-4,5-dihydro-1H-pyrazole-1-carbimidoyl chloride (0.25 g, 0.47 mmol, 1.0 eq.). After stirring at rt for 18 h, solvents were removed under reduced pressure. The residue was diluted with dichloromethane (20 mL) and washed with hydrogen chloride 1 N solution (15 mL) and brine (20 mL). The organic extract was dried over magnesium sulfate, filtered, and concentrated in vacuo. The residue was chromatographed on silica eluting with methanol/dichloromethane mixtures (0/100 to 10/90, v/v) to give the expected compound as a colorless oil (0.27 g, 0.35 mmol, 72% yield). LC-MS (ESI+): 727.0 (M-^{t}Bu+H⁺), LC-MS (ESI-): 781.2 (M−H⁺).

### Synthesis of Compounds 181 and 182

Hydrogen chloride 4M in dioxane (0.43 mL, 1.7 mmol, 5.0 eq.) was added to a stirring solution of tert-butyl (E)-4-((2-(3-(4-chlorophenyl)-4-phenyl-N′-((4-(trifluoromethyl) phenyl)sulfonyl)-4,5-dihydro-1H-pyrazole-1-carboximidamido)ethyl)sulfonyl)piperazine-1-carboxylate (0.27 mg, 0.34 mmol, 181.1) in dichloromethane (3 mL). After stirring at rt for 18 h, solvents were removed under reduced pressure. The residue was chromatographed on silica eluting with methanol/dichloromethane mixtures (0/100 to 10/90, v/v) to give a yellowish oil which was further subjected to SFC purification to resolve the two enantiomers: Compound 181 (19.0 mg, 0.03 mmol, 8% yield, 96% purity, 99% ee, OR: −50.2) and Compound 182 (35.9 mg, 0.05 mmol, 15% yield, 99% purity, 99% ee, OR: +93.4).

Method of SFC: SFC purification was performed on a Jasco 4000 Parallel SFC System (4× Channels 3×UV+1×DAD) using an Phenomenex column (Lux Amylose-A) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 181: ¹H NMR (DMSO-d₆, 400 MHz) δ 8.27 (br s, 1H), 8.20 (s, 1H), 8.03 (d, J=8.2 Hz, 2H), 7.86 (d, J=8.4 Hz, 2H), 7.73 (d, J=8.6 Hz, 2H), 7.46 (d, J=8.7 Hz, 2H), 7.30-7.37 (m, 2H), 7.18-7.30 (m, 3H), 5.11 (dd, J=11.1, 4.4 Hz, 1H), 4.55 (t, J=11.1 Hz, 1H), 4.07 (br dd, J=11.1, 4.3 Hz, 1H), 3.65-3.72 (m, 2H), 3.26-3.27 (m, 2H), 2.98 (br d, J=5.0 Hz, 4H), 2.67-2.71 ppm (m, 4H). LC-MS (ESI+): 683.1 (M+H⁺).

Compound 182: ¹H NMR (DMSO-d₆, 400 MHz) δ 8.23-8.32 (m, 1H), 8.04 (d, J=8.2 Hz, 2H), 7.86 (d, J=8.4 Hz, 2H), 7.74 (d, J=8.6 Hz, 2H), 7.46 (d, J=8.6 Hz, 2H), 7.30-7.37 (m, 2H), 7.20-7.29 (m, 3H), 5.11 (dd, J=11.2, 4.3 Hz, 1H), 4.55 (t, J=11.2 Hz, 1H), 4.07 (dd, J=11.1, 4.3 Hz, 1H), 3.61-3.76 (m, 2H), 3.25-3.28 (m, 3H), 2.93-3.08 (m, 4H), 2.65-2.77 ppm (m, 4H). LC-MS (ESI+): 683.1 (M+H⁺).

### Synthesis of (S,E)-3-(4-chlorophenyl)-N′-((4-chlorophenyl)sulfonyl)-4-phenyl-N-(2-(sulfamoylamino)ethyl)-4,5-dihydro-1H-pyrazole-1-carboximidamide (Compound 183) & (R,E)-3-(4-chlorophenyl)-N′-((4-chlorophenyl)sulfonyl)-4-phenyl-N-(2-(sulfamoylamino)ethyl)-4,5-dihydro-1H-pyrazole-1-carboximidamide (Compound 184)

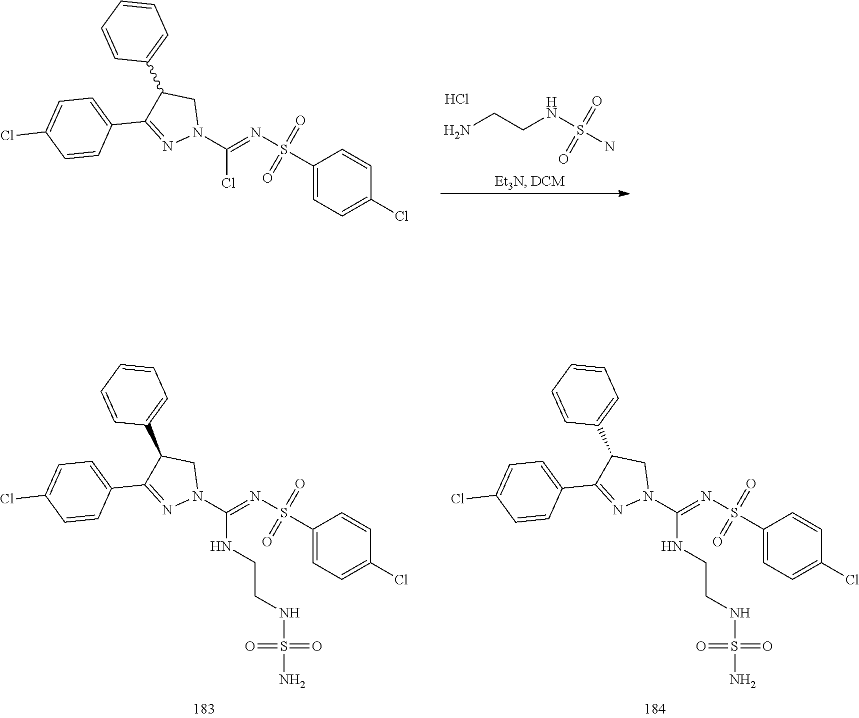 [SMILES: O=S(=O)(/N=C(\Cl)N1CC(c2ccccc2)C(c2ccc(Cl)cc2)=N1)c1ccc(Cl)cc1 ; Cl ; NCCNS(N)(=O)=O ; NS(=O)(=O)NCCN/C(=N\S(=O)(=O)c1ccc(Cl)cc1)N1C[C@H](c2ccccc2)C(c2ccc(Cl)cc2)=N1 ; NS(=O)(=O)NCCN/C(=N\S(=O)(=O)c1ccc(Cl)cc1)N1C[C@@H](c2ccccc2)C(c2ccc(Cl)cc2)=N1]

To a stirred solution of 1-amino-2-(sulfamoylamino)ethane hydrochloride (133 mg, 0.76 mmol, 1.5 eq.) and triethylamine (0.35 mL, 2.53 mmol, 5.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0 eq.). After stirring at rt for 18 h, solvents were removed under reduced pressure. The residue was chromatographed on silica eluting with ethyl acetate/heptane mixtures (0/100 to 60/40, v/v) to give a colorless oil, which was further subjected to SFC purification to afford the two enantiomers. SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 183: (74.9 mg, 0.13 mmol, 25% yield, 99% purity, 99% ee, OR: −96.9). 1H NMR (DMSO-d₆, 400 MHz) δ 8.01 (br s, 1H), 7.82 (d, J=8.6 Hz, 2H), 7.73 (d, J=8.5 Hz, 2H), 7.53 (d, J=8.6 Hz, 2H), 7.44 (d, J=8.6 Hz, 2H), 7.3-7.4 (m, 2H), 7.2-7.3 (m, 3H), 6.75 (br t, J=5.8 Hz, 1H), 6.60 (s, 2H), 5.07 (dd, J=4.2, 11.1 Hz, 1H), 4.51 (t, J=11.2 Hz, 1H), 4.04 (br dd, J=4.0, 11.1 Hz, 1H), 3.48 (br d, J=5.4 Hz, 2H), 3.10 (q, J=5.9 Hz, 2H). LC-MS (ESI+): 595.0 (M+H⁺)

Compound 184: (72.6 mg, 0.12 mmol, 24% yield, 99% purity, 99% ee, OR: +143.3). 1H NMR (DMSO-d₆, 400 MHz) δ 8.01 (br s, 1H), 7.82 (d, J=8.6 Hz, 2H), 7.73 (d, J=8.6 Hz, 2H), 7.53 (d, J=8.6 Hz, 2H), 7.44 (d, J=8.6 Hz, 2H), 7.3-7.4 (m, 2H), 7.2-7.3 (m, 3H), 6.75 (br t, J=5.7 Hz, 1H), 6.60 (s, 2H), 5.07 (dd, J=4.3, 11.2 Hz, 1H), 4.51 (t, J=11.2 Hz, 1H), 4.04 (br dd, J=4.1, 10.9 Hz, 1H), 3.48 (br d, J=5.1 Hz, 2H), 3.10 (q, J=5.9 Hz, 2H). LC-MS (ESI+): 595.0 (M+H⁺)

### Synthesis of (4S,E)-3-(4-chlorophenyl)-N′-((4-chlorophenyl)sulfonyl)-4-phenyl-N-(2-(sulfamoylamino)propyl)-4,5-dihydro-1H-pyrazole-1-carboximidamide (Compound 185) & (4R,E)-3-(4-chlorophenyl)-N′-((4-chlorophenyl)sulfonyl)-4-phenyl-N-(2-(sulfamoylamino)propyl)-4,5-dihydro-1H-pyrazole-1-carboximidamide (Compound 186)

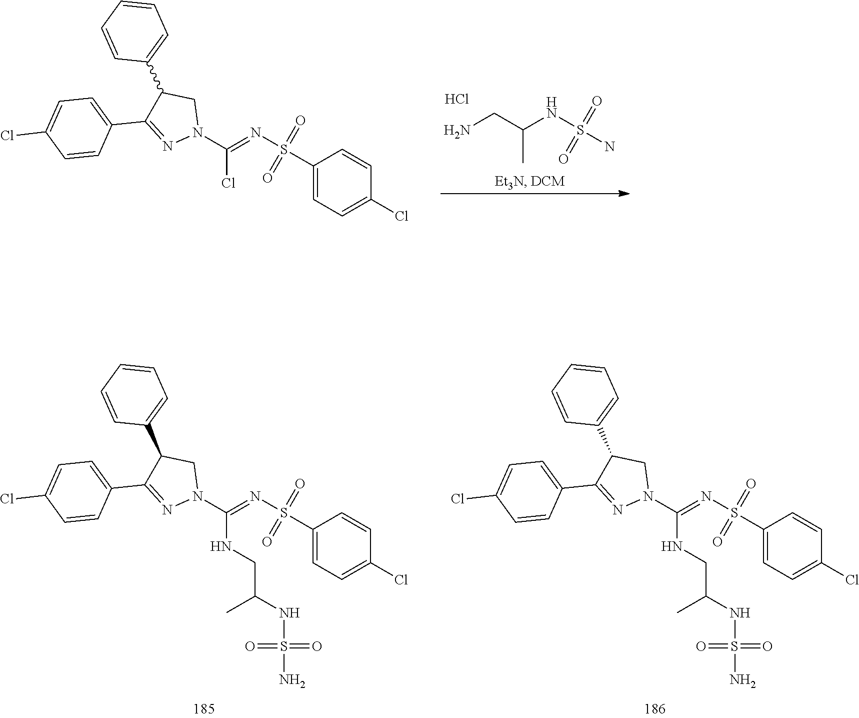

To a stirred solution of 1-Amino-2-(sulfamoylamino)propane hydrochloride (144 mg, 0.76 mmol, 1.5 eq.) and triethylamine (0.35 mL, 2.53 mmol, 5.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0 eq.). After stirring at rt for 18 h, solvents were removed under reduced pressure. The residue was chromatographed on silica eluting with ethyl acetate/heptane mixtures (0/100 to 70/30, v/v) to obtain a colorless oil, which was further subjected to SFC purification to resolve the enantiomers.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 185: (76.5 mg, 0.13 mmol, 25% yield, 99% purity, 99% ee, OR: −117.0). 1H NMR (DMSO-d₆, 400 MHz) δ 7.90 (br s, 1H), 7.81 (d, J=8.6 Hz, 2H), 7.73 (d, J=8.5 Hz, 2H), 7.53 (d, J=8.6 Hz, 2H), 7.44 (d, J=8.6 Hz, 2H), 7.3-7.4 (m, 2H), 7.2-7.3 (m, 3H), 6.75 (br d, J=7.5 Hz, 1H), 6.58 (s, 2H), 5.07 (dd, J=4.2, 11.1 Hz, 1H), 4.52 (t, J=11.2 Hz, 1H), 4.02 (br dd, J=4.1, 11.2 Hz, 1H), 3.51 (br d, J=6.7 Hz, 1H), 3.4-3.5 (m, 1H), 3.2-3.3 (m, 1H), 1.07 (d, J=6.5 Hz, 3H). LC-MS (ESI+): 609.0 (M+H⁺)

Compound 186: (76.4 mg, 0.13 mmol, 25% yield, 99% purity, 99% ee, OR: +124.5). 1H NMR (DMSO-d₆, 400 MHz) δ 7.89 (br s, 1H), 7.81 (d, J=8.5 Hz, 2H), 7.73 (d, J=8.5 Hz, 2H), 7.54 (d, J=8.6 Hz, 2H), 7.44 (d, J=8.6 Hz, 2H), 7.3-7.4 (m, 2H), 7.2-7.3 (m, 3H), 6.7-6.8 (m, 1H), 6.59 (br s, 2H), 5.07 (dd, J=4.2, 11.1 Hz, 1H), 4.51 (t, J=11.2 Hz, 1H), 4.05 (br dd, J=4.0, 11.1 Hz, 1H), 3.53 (td, J=6.4, 12.9 Hz, 1H), 3.43 (br d, J=12.3 Hz, 1H), 3.27 (br s, 1H), 1.08 (d, J=6.5 Hz, 3H). LC-MS (ESI+): 609.0 (M+H⁺)

### Synthesis of (S,E)-3-(4-chlorophenyl)-N′-((4-chlorophenyl)sulfonyl)-N-(2-(methyl(sulfamoyl)amino)ethyl)-4-phenyl-4,5-dihydro-1H-pyrazole-1-carboximidamide (Compound 187) and (R,E)-3-(4-chlorophenyl)-N′-((4-chlorophenyl)sulfonyl)-N-(2-(methyl(sulfamoyl)amino)ethyl)-4-phenyl-4,5-dihydro-1H-pyrazole-1-carboximidamide (Compound 188)

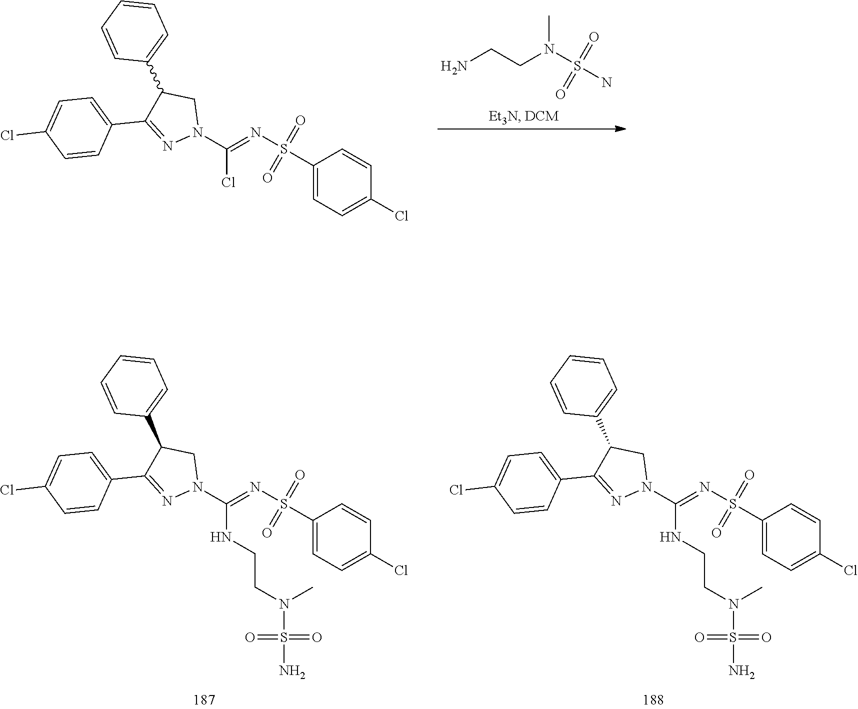

To a stirred solution of 2-(methyl(sulfamoyl)amino)ethyl)amine (116 mg, 0.76 mmol, 1.5 eq.) and triethylamine (0.35 mL, 2.53 mmol, 5.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0 eq.). After stirring at rt for 18 h, solvents were removed under reduced pressure. The residue was chromatographed on silica eluting with ethyl acetate/heptane mixtures (0/100 to 80/20, v/v) to obtain a colorless oil, which was further subjected to SFC purification to resolve the enantiomers.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—EtOH+0.1% diethylamine, at 35° C., BPR 100 bar. Acquisition frequency was set to 220 nm for the DAD detector.

Compound 187: (25.4 mg, 0.04 mmol, 8% yield, 99% purity, 99% ee, OR: −99.6). 1H NMR (DMSO-d₆, 400 MHz) δ 8.12 (br s, 1H), 7.80-7.85 (m, 2H), 7.75 (d, J=8.7 Hz, 2H), 7.51-7.56 (m, 2H), 7.45 (d, J=8.7 Hz, 2H), 7.30-7.36 (m, 2H), 7.20-7.28 (m, 3H), 6.76 (s, 2H), 5.08 (dd, J=11.2, 4.3 Hz, 1H), 4.52 (t, J=11.2 Hz, 1H), 4.05 (br dd, J=11.0, 4.1 Hz, 1H), 3.47 (br s, 2H), 3.10 (br t, J=6.8 Hz, 2H), 2.56 ppm (s, 3H). LC-MS (ESI+): 609.0 (M+H⁺)

Compound 188: (30.0 mg, 0.05 mmol, 10% yield, 99% purity, 99% ee, OR: +30.9). 1H NMR (DMSO-d₆, 400 MHz) δ 8.06-8.18 (m, 1H), 7.80-7.86 (m, 2H), 7.75 (d, J=8.6 Hz, 2H), 7.51-7.56 (m, 2H), 7.45 (d, J=8.6 Hz, 2H), 7.30-7.36 (m, 2H), 7.21-7.28 (m, 3H), 6.76 (s, 2H), 5.08 (dd, J=11.2, 4.3 Hz, 1H), 4.52 (t, J=11.2 Hz, 1H), 4.05 (br dd, J=11.1, 4.1 Hz, 1H), 3.47 (br d, J=3.7 Hz, 2H), 3.10 (br t, J=6.8 Hz, 2H), 2.56 ppm (s, 3H). LC-MS (ESI+): 609.0 (M+H⁺).

### Synthesis of (S,E)-3-(4-chlorophenyl)-N′-((4-chlorophenyl)sulfonyl)-N-methyl-N-(2-(methyl(sulfamoyl)amino)ethyl)-4-phenyl-4,5-dihydro-1H-pyrazole-1-carboximidamide (Compound 189) and (R,E)-3-(4-chlorophenyl)-N′-((4-chlorophenyl)sulfonyl)-N-methyl-N-(2-(methyl(sulfamoyl)amino)ethyl)-4-phenyl-4,5-dihydro-1H-pyrazole-1-carboximidamide (Compound 190)

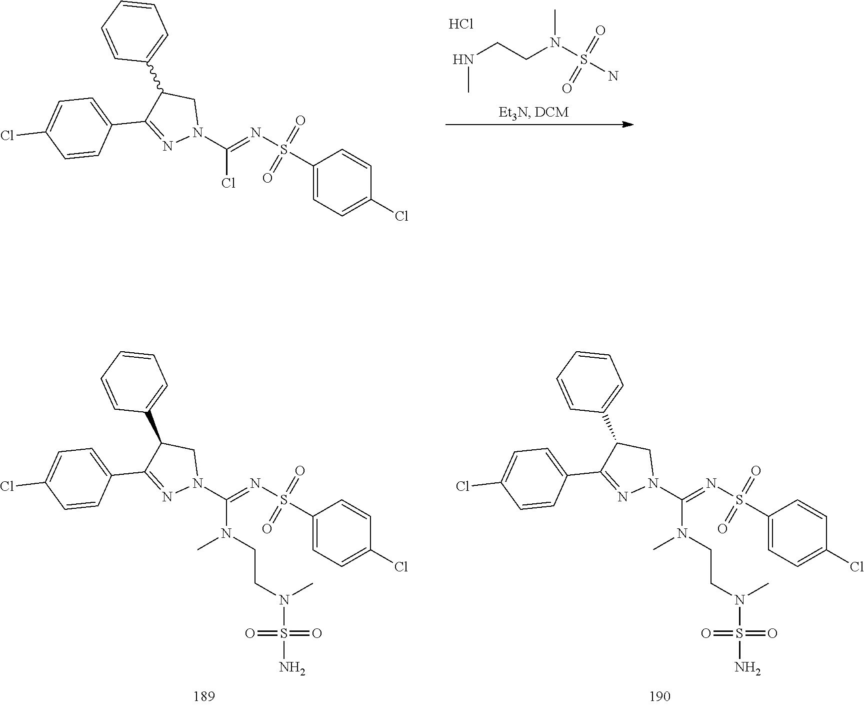

To a stirred solution of methyl(2-(methyl(sulfamoyl)amino)ethyl)amine hydrochloride (0.66 g, 3.2 mmol, 2.0 eq.) and triethylamine (0.7 mL, 4.9 mmol, 3.0 eq.) in dichloromethane (10 mL) was added (Z)-3-(4-chlorophenyl)-N-((4-chlorophenyl)sulfonyl)-4-phenyl-4,5-dihydro-1H-pyrazole-1-carbimidoyl chloride (0.8 g, 1.6 mmol, 1.0 eq.). After stirring at rt for 18 h, solvents were removed under reduced pressure. The residue was chromatographed on silica eluting with ethyl acetate/heptane mixtures (0/100 to 70/30, v/v) to afford a colorless oil, which was further subjected to SFC purification to resolve the enantiomers.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—MeOH+0.1% diethylamine, at 35° C., BPR 100 bar. Acquisition frequency was set to 220 nm for the DAD detector.

Compound 189: (32.4 mg, 0.05 mmol, 3% yield, 96% purity, 99% ee, OR: −103.8). 1H NMR (DMSO-d₆, 400 MHz) δ 7.8-7.8 (m, 2H), 7.5-7.6 (m, 4H), 7.39 (d, J=8.6 Hz, 2H), 7.3-7.3 (m, 4H), 7.24 (dt, J=2.6, 6.1 Hz, 1H), 6.78 (s, 2H), 5.03 (dd, J=5.3, 11.4 Hz, 1H), 4.54 (t, J=11.2 Hz, 1H), 3.85 (dd, J=5.3, 10.9 Hz, 1H), 3.5-3.7 (m, 2H), 3.19 (br t, J=6.5 Hz, 2H), 3.11 (s, 3H), 2.55 (s, 3H). LC-MS (ESI+): 623.1 (M+H+)

Compound 190: (32.6 mg, 0.05 mmol, 3% yield, 94% purity, 99% ee, OR: +94.8). 1H NMR (DMSO-d₆, 400 MHz) δ 7.79 (d, J=8.5 Hz, 2H), 7.5-7.6 (m, 4H), 7.39 (d, J=8.6 Hz, 2H), 7.30 (s, 4H), 7.24 (dt, J=2.5, 6.1 Hz, 1H), 6.78 (br s, 2H), 5.03 (dd, J=5.2, 11.3 Hz, 1H), 4.54 (t, J=11.2 Hz, 1H), 3.85 (dd, J=5.3, 10.8 Hz, 1H), 3.5-3.7 (m, 2H), 3.19 (br t, J=6.5 Hz, 2H), 3.11 (s, 3H), 2.55 (s, 3H). LC-MS (ESI+): 623.1 (M+H+)

### Synthesis of (S,E)-3-(4-chlorophenyl)-N′-((4-chlorophenyl)sulfonyl)-4-phenyl-N-(1-sulfamoylazetidin-3-yl)-4,5-dihydro-1H-pyrazole-1-carboximidamide (Compound 191) & (R,E)-3-(4-chlorophenyl)-N′-((4-chlorophenyl)sulfonyl)-4-phenyl-N-(1-sulfamoylazetidin-3-yl)-4,5-dihydro-1H-pyrazole-1-carboximidamide (Compound 192)

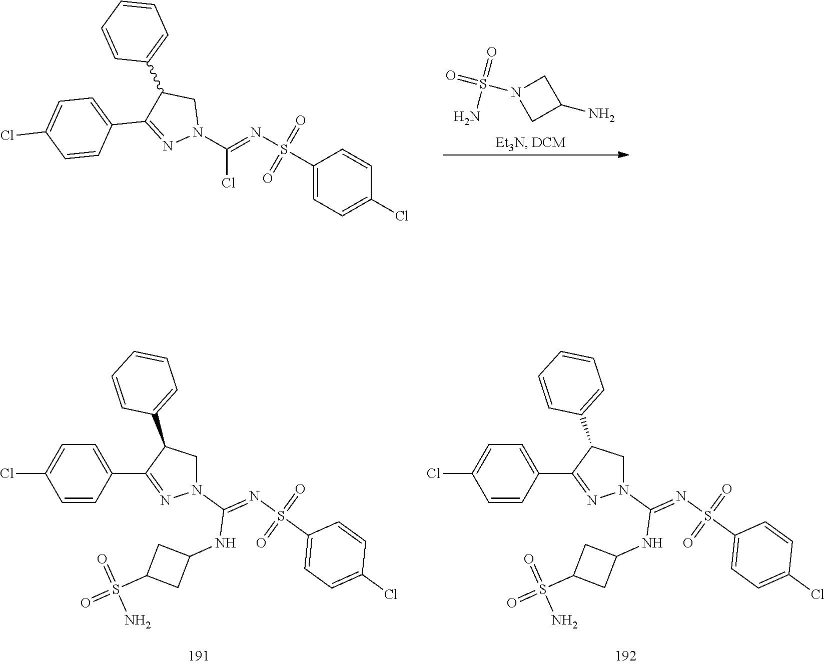 [SMILES: O=S(=O)(/N=C(\Cl)N1CC(c2ccccc2)C(c2ccc(Cl)cc2)=N1)c1ccc(Cl)cc1 ; NC1CN(S(N)(=O)=O)C1 ; NS(=O)(=O)C1CC(N/C(=N\S(=O)(=O)c2ccc(Cl)cc2)N2C[C@H](c3ccccc3)C(c3ccc(Cl)cc3)=N2)C1 ; NS(=O)(=O)C1CC(N/C(=N\S(=O)(=O)c2ccc(Cl)cc2)N2C[C@@H](c3ccccc3)C(c3ccc(Cl)cc3)=N2)C1]

To a stirred solution of 3-aminoazetidine-1-sulfonamide trifluoroacetic salt (0.12 g, 0.67 mmol, 1.1 eq.) and triethylamine (0.42 mL, 3.04 mmol, 5.0 eq.) in dichloromethane (6 mL) was added (Z)-3-(4-chlorophenyl)-N-((4-chlorophenyl)sulfonyl)-4-phenyl-4,5-dihydro-1H-pyrazole-1-carbimidoyl chloride (0.3 g, 0.61 mmol, 1.0 eq.). After stirring at rt for 18 h, the reaction mixture was diluted with dichloromethane (20 mL) and washed with ammonium chloride (20 mL). The organic phase was dried over magnesium sulfate, filtered, and concentrated in vacuo. The residue was chromatographed on silica eluting with ethyl acetate/heptane mixtures (0/100 to 60/40, v/v) to afford a colorless oil, which was further subjected to SFC purification to resolve both enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—EtOH+0.1% diethylamine, at 35° C., BPR 100 bar. Acquisition frequency was set to 220 nm for the DAD detector.

Compound 191: (13.7 mg, 0.022 mmol, 4% yield, 99% purity, 99% ee, OR: −10.0). 1H NMR (DMSO-d₆, 400 MHz) δ 8.42 (br d, J=3.1 Hz, 1H), 7.73-7.84 (m, 4H), 7.52-7.58 (m, 2H), 7.43-7.49 (m, 2H), 7.30-7.36 (m, 2H), 7.19-7.28 (m, 3H), 6.98 (s, 2H), 5.07 (br dd, J=10.8, 3.1 Hz, 1H), 4.44-4.55 (m, 2H), 3.96 (br dd, J=11.2, 3.0 Hz, 1H), 3.87 (q, J=7.7 Hz, 2H), 3.77 ppm (dt, J=13.7, 6.8 Hz, 2H). LC-MS (ESI+): 607.0 (M+H+)

Compound 192: (14.3 mg, 0.023 mmol, 4% yield, 99% purity, 99% ee, OR: +8.0). 1H NMR (DMSO-d₆, 400 MHz) δ 8.42 (br s, 1H), 7.74-7.83 (m, 4H), 7.53-7.58 (m, 2H), 7.44-7.48 (m, 2H), 7.31-7.36 (m, 2H), 7.20-7.28 (m, 3H), 6.98 (s, 2H), 5.07 (dd, J=11.2, 4.4 Hz, 1H), 4.45-4.54 (m, 2H), 3.97 (dd, J=11.2, 4.4 Hz, 1H), 3.87 (q, J=7.9 Hz, 2H), 3.73-3.81 ppm (m, 2H). LC-MS (ESI+): 607.0 (M+H+)

### Synthesis of (S,E)-3-(4-chlorophenyl)-4-phenyl-N-(2-(sulfamoylamino)ethyl)-N′-((4-(trifluoromethyl)phenyl)sulfonyl)-4,5-dihydro-1H-pyrazole-1-carboximidamide (Compound 193) & (R,E)-3-(4-chlorophenyl)-4-phenyl-N-(2-(sulfamoylamino)ethyl)-N′-((4-(trifluoromethyl)phenyl)sulfonyl)-4,5-dihydro-1H-pyrazole-1-carboximidamide (Compound 194)

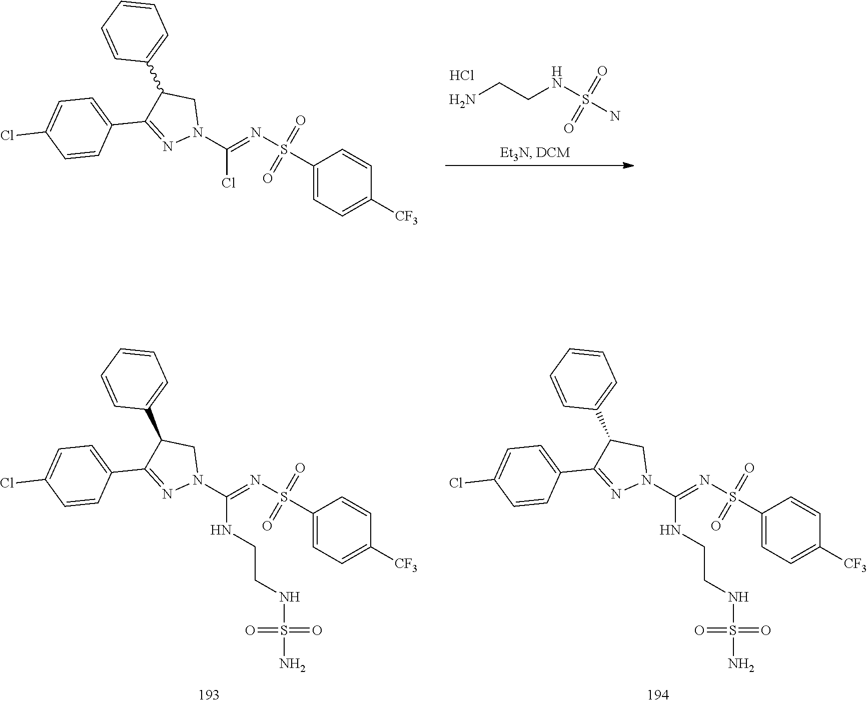 [SMILES: O=S(=O)(/N=C(\Cl)N1CC(c2ccccc2)C(c2ccc(Cl)cc2)=N1)c1ccc(C(F)(F)F)cc1 ; Cl ; NCCNS(N)(=O)=O ; NS(=O)(=O)NCCN/C(=N\S(=O)(=O)c1ccc(C(F)(F)F)cc1)N1C[C@H](c2ccccc2)C(c2ccc(Cl)cc2)=N1 ; NS(=O)(=O)NCCN/C(=N\S(=O)(=O)c1ccc(C(F)(F)F)cc1)N1C[C@@H](c2ccccc2)C(c2ccc(Cl)cc2)=N1]

To a stirred solution of 1-amino-2-(sulfamoylamino)ethane hydrochloride (125 mg, 0.71 mmol, 1.5 eq.) and triethylamine (0.33 mL, 2.37 mmol, 5.0 eq.) in dichloromethane (5 mL) was added (Z)-3-(4-chlorophenyl)-4-phenyl-N-((4-(trifluoromethyl)phenyl)sulfonyl)-4,5-dihydro-1H-pyrazole-1-carbimidoyl chloride (250 mg, 0.47 mmol, 1.0 eq.). After stirring at rt for 18 h, solvents were removed under reduced pressure and the residue was chromatographed on silica eluting with ethyl acetate/heptane mixtures (0/100 to 60/40, v/v) to obtain a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 193: (45.3 mg, 0.072 mmol, 15% yield, 96% purity, 99% ee, OR: +81.7). 1H NMR (DMSO-d₆, 400 MHz) δ 8.05-8.11 (m, 1H), 8.02 (d, J=8.2 Hz, 2H), 7.85 (d, J=8.4 Hz, 2H), 7.72 (d, J=8.6 Hz, 2H), 7.44 (d, J=8.7 Hz, 2H), 7.30-7.36 (m, 2H), 7.21-7.28 (m, 3H), 6.76 (br t, J=5.9 Hz, 1H), 6.61 (s, 2H), 5.08 (dd, J=11.1, 4.2 Hz, 1H), 4.53 (t, J=11.2 Hz, 1H), 4.03 (br dd, J=10.9, 4.2 Hz, 1H), 3.48 (br d, J=5.7 Hz, 2H), 3.11 (q, J=6.3 Hz, 2H) ppm. LC-MS (ESI+): 629.0 (M+H+)

Compound 194: (39.0 mg, 0.062 mmol, 13% yield, 98% purity, 99% ee, OR: −81.5). 1H NMR (DMSO-d₆, 400 MHz) δ 8.05-8.11 (m, 1H), 8.02 (d, J=8.2 Hz, 2H), 7.85 (d, J=8.3 Hz, 2H), 7.72 (d, J=8.6 Hz, 2H), 7.44 (d, J=8.7 Hz, 2H), 7.30-7.36 (m, 2H), 7.21-7.28 (m, 3H), 6.76 (t, J=6.0 Hz, 1H), 6.61 (s, 2H), 5.08 (dd, J=11.2, 4.3 Hz, 1H), 4.53 (t, J=11.2 Hz, 1H), 4.03 (br dd, J=11.1, 4.2 Hz, 1H), 3.48 (br d, J=5.7 Hz, 2H), 3.11 (q, J=6.3 Hz, 2H) ppm. LC-MS (ESI+): 629.1 (M+H+).

### Synthesis of (S,E)-N′-((4-chlorophenyl)sulfonyl)-3-(4-fluorophenyl)-4-phenyl-N-(2-(sulfamoylamino)ethyl)-4,5-dihydro-1H-pyrazole-1-carboximidamide (Compound 195) & (R,E)-N′-((4-chlorophenyl)sulfonyl)-3-(4-fluorophenyl)-4-phenyl-N-(2-(sulfamoylamino)ethyl)-4,5-dihydro-1H-pyrazole-1-carboximidamide (Compound 196)

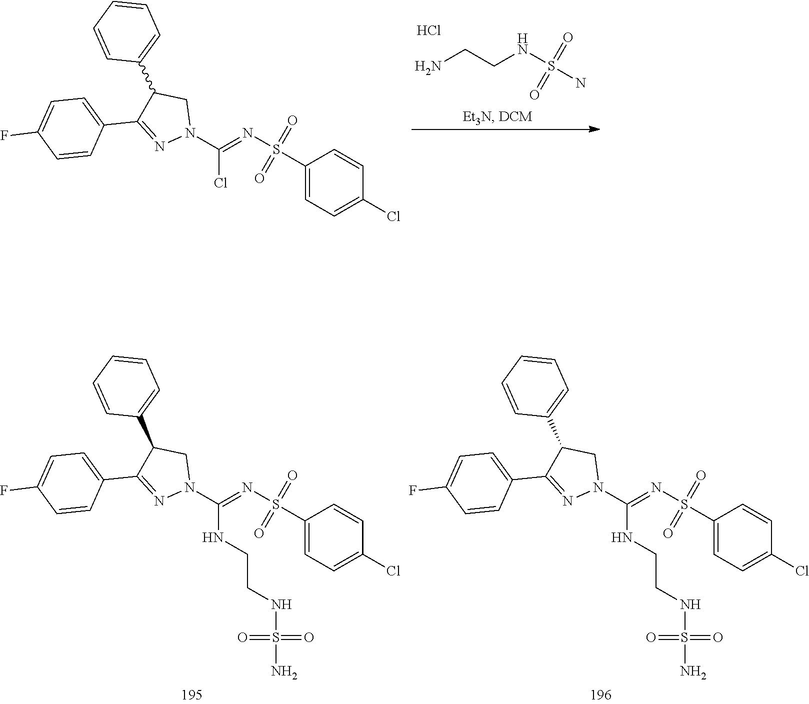 [SMILES: O=S(=O)(/N=C(\Cl)N1CC(c2ccccc2)C(c2ccc(F)cc2)=N1)c1ccc(Cl)cc1 ; Cl ; NCCNS(N)(=O)=O ; NS(=O)(=O)NCCN/C(=N\S(=O)(=O)c1ccc(Cl)cc1)N1C[C@H](c2ccccc2)C(c2ccc(F)cc2)=N1 ; NS(=O)(=O)NCCN/C(=N\S(=O)(=O)c1ccc(Cl)cc1)N1C[C@@H](c2ccccc2)C(c2ccc(F)cc2)=N1]

To a stirred solution of 1-amino-2-(sulfamoylamino)ethane hydrochloride (111 mg, 0.63 mmol, 1.5 eq.) and triethylamine (0.29 mL, 2.1 mmol, 5.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (200 mg, 0.42 mmol, 1.0 eq.). After stirring at rt for 18 h, solvents were removed under reduced pressure and the crude was chromatographed on silica eluting with ethyl acetate/heptane mixtures (0/100 to 80/20, v/v) to obtain a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—iPrOH+0.1% diethylamine, at 35° C., BPR 100 bar. Acquisition frequency was set to 220 nm for the DAD detector.

Compound 195: (16.0 mg, 0.023 mmol, 7% yield, 96% purity, 99% ee, OR: −86.5). 1H NMR (DMSO-d₆, 400 MHz) δ 7.99 (br s, 1H), 7.76-7.86 (m, 4H), 7.51-7.56 (m, 2H), 7.31-7.36 (m, 2H), 7.20-7.29 (m, 5H), 6.75 (t, J=5.91 Hz, 1H), 6.60 (s, 2H), 5.07 (dd, J=11.11, 4.24 Hz, 1H), 4.50 (t, J=11.21 Hz, 1H), 4.04 (br dd, J=11.06, 4.01 Hz, 1H), 3.48 (br d, J=5.82 Hz, 2H), 3.10 (q, J=6.20 Hz, 2H) ppm. LC-MS (ESI+): 579.1 (M+H+)

Compound 196: (16.5 mg, 0.023 mmol, 7% yield, 99% purity, 99% ee, OR: +124.9). 1H NMR (DMSO-d₆, 400 MHz) δ 7.99 (br s, 1H), 7.76-7.85 (m, 4H), 7.51-7.56 (m, 2H), 7.30-7.36 (m, 2H), 7.20-7.28 (m, 5H), 6.75 (br t, J=6.0 Hz, 1H), 6.61 (s, 2H), 5.07 (dd, J=11.1, 4.2 Hz, 1H), 4.50 (t, J=11.2 Hz, 1H), 4.04 (br dd, J=11.0, 4.2 Hz, 1H), 3.48 (br d, J=5.7 Hz, 2H), 3.10 ppm (q, J=6.2 Hz, 2H). LC-MS (ESI+): 579.1 (M+H+)

### Synthesis of (S,E)-N′-((4-chlorophenyl)sulfonyl)-3-(4-fluorophenyl)-4-phenyl-N-((S)-2-(sulfamoylamino)propyl)-4,5-dihydro-1H-pyrazole-1-carboximidamide (Compound 197) & (R,E)-N′-((4-chlorophenyl)sulfonyl)-3-(4-fluorophenyl)-4-phenyl-N-((S)-2-(sulfamoylamino)propyl)-4,5-dihydro-1H-pyrazole-1-carboximidamide (Compound 198)

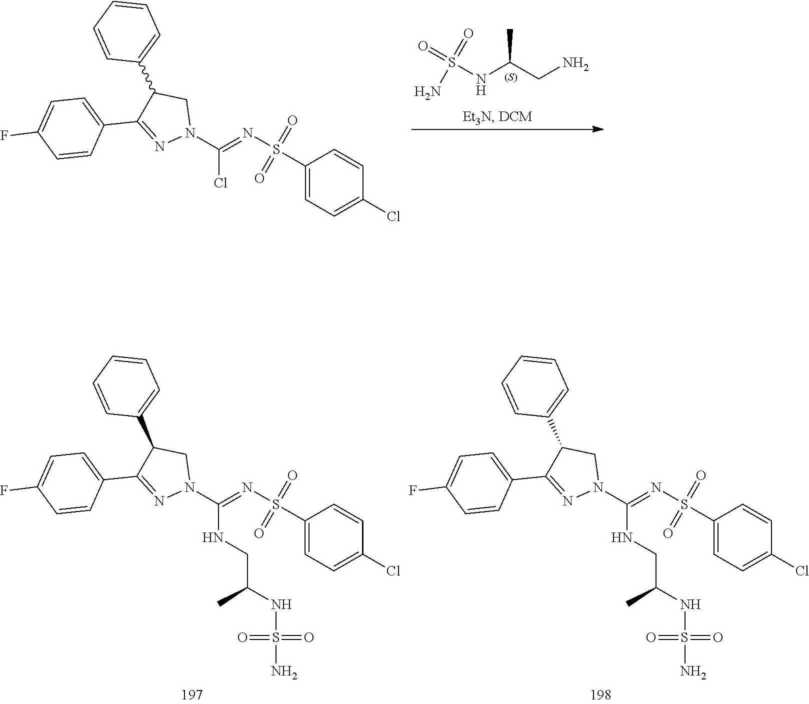

To a stirred solution of (S)-(1-Aminopropan-2-yl)sulfamide (121 mg, 0.79 mmol, 1.5 eq.) and triethylamine (0.22 mL, 1.57 mmol, 3.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (250 mg, 0.52 mmol, 1.0 eq.). After stirring at rt for 18 h, solvents were removed under reduced pressure and the crude was chromatographed on silica eluting with ethyl acetate/heptane mixtures (0/100 to 80/20) to obtain a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 197: (10.4 mg, 0.017 mmol, 3% yield, 97% purity, 99% ee, OR: −68.3). 1H NMR (CDCl₃, 400 MHz) δ 7.86 (d, J=8.6 Hz, 2H), 7.58 (dd, J=8.8, 5.3 Hz, 2H), 7.36-7.44 (m, 3H), 7.28-7.35 (m, 3H), 7.13 (br d, J=6.9 Hz, 2H), 6.97 (t, J=8.6 Hz, 2H), 5.10 (br d, J=8.3 Hz, 1H), 5.05 (s, 2H), 4.66 (dd, J=11.3, 5.5 Hz, 1H), 4.50 (t, J=11.5 Hz, 1H), 4.04 (dd, J=11.6, 5.6 Hz, 1H), 3.76-3.95 (m, 2H), 3.55 (ddd, J=14.0, 8.8, 5.6 Hz, 1H), 1.32 (d, J=6.6 Hz, 3H) ppm. LC-MS (ESI+): 593.1 (M+H+)

Compound 198: (8.1 mg, 0.014 mmol, 3% yield, 99% purity, 99% ee, OR: +37.2). 1H NMR (MeOD-d₄, 400 MHz) δ 7.87 (d, J=8.6 Hz, 2H), 7.78 (dd, J=8.9, 5.3 Hz, 2H), 7.48 (d, J=8.7 Hz, 3H), 7.24-7.37 (m, 3H), 7.19-7.23 (m, 2H), 7.05 (t, J=8.8 Hz, 2H), 4.93 (dd, J=11.2, 4.5 Hz, 1H), 4.50 (t, J=11.3 Hz, 1H), 4.02 (dd, J=11.3, 4.6 Hz, 1H), 3.62-3.73 (m, 2H), 3.36-3.46 (m, 1H), 1.24 (d, J=6.5 Hz, 3H) ppm. LC-MS (ESI+): 593.1 (M+H+)

### Synthesis of (S,E)-3-(4-chlorophenyl)-N′-((4-chlorophenyl)sulfonyl)-4-phenyl-N-((S)-2-(sulfamoylamino)propyl)-4,5-dihydro-1H-pyrazole-1-carboximidamide (Compound 199) & (R,E)-3-(4-chlorophenyl)-N′-((4-chlorophenyl)sulfonyl)-4-phenyl-N-((S)-2-(sulfamoylamino)propyl)-4,5-dihydro-1H-pyrazole-1-carboximidamide (Compound 200)

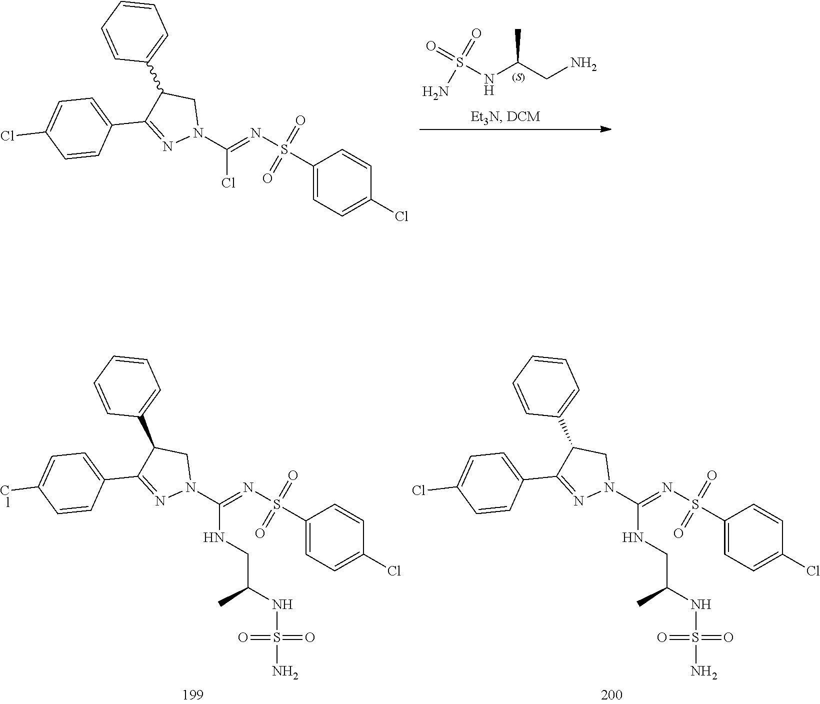

To a stirred solution of (S)-(1-Aminopropan-2-yl)sulfamide (116 mg, 0.76 mmol, 1.5 eq.) and triethylamine (0.21 mL, 1.52 mmol, 3.0 eq.) in dichloromethane (5 mL) was added (Z)-N-((4-chlorophenyl)sulfonyl)-3-(4-chlorophenyl)-4-phenyl-4,5-dihydro-1H-pyrazole-1-carbimidoyl chloride (250 mg, 0.51 mmol, 1.0 eq.). After stirring at rt for 18 h, the reaction mixture was diluted with dichloromethane (30 mL) and extracted with ammonium chloride (25 mL). The organic layer was separated, dried over magnesium sulfate, filtered, and concentrated in vacuo. The residue was purified by flash chromatography on silica eluting with ethyl acetate/heptane mixtures (0/100 to 80/20, v/v) to obtain a colorless oil, which was further subjected to SFC purification to resolve the two enantiomers as white solids.
SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 199: (7.3 mg, 0.012 mmol, 2% yield, 98% purity, 99% ee, OR: −62.1). 1H NMR (MeOD-d₄, 400 MHz) δ 7.84-7.91 (m, 2H), 7.70 (d, J=8.7 Hz, 2H), 7.45-7.52 (m, 2H), 7.26-7.35 (m, 5H), 7.19-7.23 (m, 2H), 4.93 (dd, J=11.2, 4.7 Hz, 1H), 4.51 (t, J=11.3 Hz, 1H), 4.04 (dd, J=11.3, 4.8 Hz, 1H), 3.64-3.73 (m, 2H), 3.39 (br dd, J=14.4, 9.6 Hz, 1H), 1.24 (d, J=6.5 Hz, 3H) ppm. LC-MS (ESI+): 609.06 (M+H+).

Compound 200: (10.5 mg, 0.017 mmol, 4% yield, 98% purity, 99% ee, OR: +64.1). 1H NMR (CDCl₃, 400 MHz) δ 7.85 (d, J=8.6 Hz, 2H), 7.52 (d, J=8.6 Hz, 2H), 7.46 (br s, 1H), 7.38 (d, J=8.6 Hz, 2H), 7.27-7.36 (m, 4H), 7.25 (s, 2H), 7.10-7.15 (m, 2H), 5.05 (br s, 2H), 4.64 (dd, J=11.4, 5.2 Hz, 1H), 4.50 (t, J=11.5 Hz, 1H), 4.03 (dd, J=11.5, 5.2 Hz, 1H), 3.86-3.96 (m, 1H), 3.81 (br s, 1H), 3.55 (ddd, J=13.9, 9.0, 5.1 Hz, 1H), 1.33 (d, J=6.6 Hz, 3H) ppm. LC-MS (ESI+): 609.06 (M+H+).

### Synthesis of (S,E)-N′-((4-chlorophenyl)sulfonyl)-3-(4-fluorophenyl)-4-phenyl-N-(1-sulfamoylazetidin-3-yl)-4,5-dihydro-1H-pyrazole-1-carboximidamide (201) & (R,E)-N′-((4-chlorophenyl)sulfonyl)-3-(4-fluorophenyl)-4-phenyl-N-(1-sulfamoylazetidin-3-yl)-4,5-dihydro-1H-pyrazole-1-carboximidamide (202)

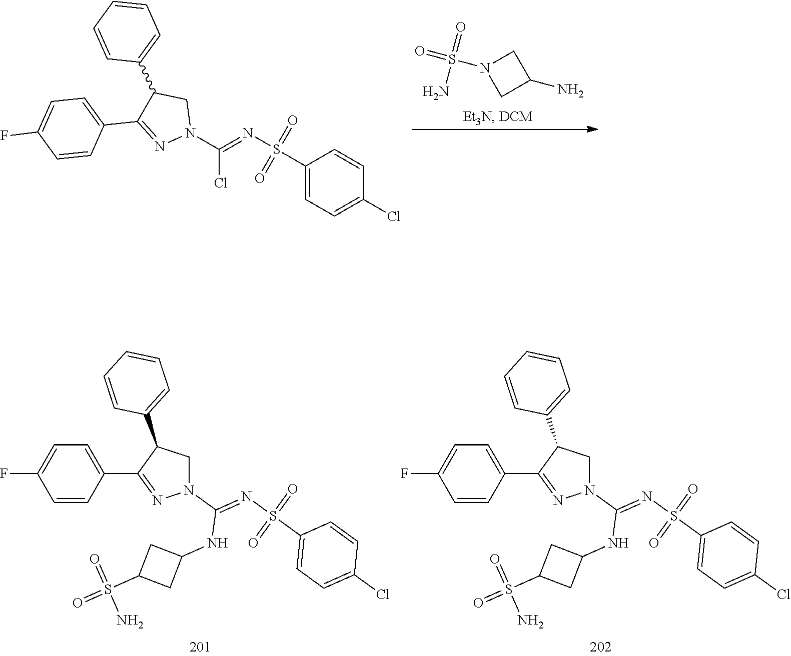 [SMILES: O=S(=O)(/N=C(\Cl)N1CC(c2ccccc2)C(c2ccc(F)cc2)=N1)c1ccc(Cl)cc1 ; NC1CN(S(N)(=O)=O)C1 ; NS(=O)(=O)C1CC(N/C(=N\S(=O)(=O)c2ccc(Cl)cc2)N2C[C@H](c3ccccc3)C(c3ccc(F)cc3)=N2)C1 ; NS(=O)(=O)C1CC(N/C(=N\S(=O)(=O)c2ccc(Cl)cc2)N2C[C@@H](c3ccccc3)C(c3ccc(F)cc3)=N2)C1]

To a stirred solution of 3-aminoazetidine-1-sulfonamide trifluoroacetic salt (0.25 g, 0.94 mmol, 2 eq.) and triethylamine (0.33 mL, 2.4 mmol, 5.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (0.225 g, 0.47 mmol, 1 eq.). After stirring at rt for 18 h, the reaction mixture was diluted with dichloromethane (20 mL) and washed with ammonium chloride (20 mL). The organic phase was dried over magnesium sulfate, filtered, and concentrated in vacuo. The residue was chromatographed on silica eluting with ethyl acetate/heptane mixtures (0/100 to 70/30, v/v) to afford a colorless oil, which was further subjected to SFC purification to resolve both enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 201: (14.8 mg, 0.025 mmol, 5% yield, 99% purity, 99% ee, OR: +99.8). 1H NMR (DMSO-d₆, 400 MHz) δ 8.39 (br d, J=5.0 Hz, 1H), 7.77-7.83 (m, 4H), 7.55 (d, J=8.5 Hz, 2H), 7.30-7.36 (m, 2H), 7.21-7.28 (m, 5H), 6.98 (s, 2H), 5.08 (dd, J=11.1, 4.3 Hz, 1H), 4.49 (br t, J=11.1 Hz, 2H), 3.97 (dd, J=11.3, 4.3 Hz, 1H), 3.87 (q, J=8.0 Hz, 2H), 3.73-3.81 (m, 2H) ppm. LC-MS (ESI+): 591.1 (M+H+).

Compound 202: (13.9 mg, 0.023 mmol, 5% yield, 99% purity, 99% ee, OR: −108.5). 1H NMR (DMSO-d₆, 400 MHz) δ 8.39 (br s, 1H), 7.76-7.84 (m, 4H), 7.55 (d, J=8.6 Hz, 2H), 7.30-7.37 (m, 2H), 7.20-7.27 (m, 5H), 6.98 (s, 2H), 5.07 (dd, J=11.1, 4.2 Hz, 1H), 4.49 (br t, J=11.1 Hz, 2H), 3.97 (dd, J=11.3, 4.2 Hz, 1H), 3.87 (q, J=8.1 Hz, 2H), 3.73-3.81 (m, 2H) ppm. LC-MS (ESI+): 591.0 (M+H+).

### Synthesis of (S,E)-N′-((4-chlorophenyl)sulfonyl)-3-(4-fluorophenyl)-N-(2-(methyl(sulfamoyl)amino)ethyl)-4-phenyl-4,5-dihydro-1H-pyrazole-1-carboximidamide (Compound 203) & (R,E)-N′-((4-chlorophenyl)sulfonyl)-3-(4-fluorophenyl)-N-(2-(methyl(sulfamoyl)amino)ethyl)-4-phenyl-4,5-dihydro-1H-pyrazole-1-carboximidamide (Compound 204)

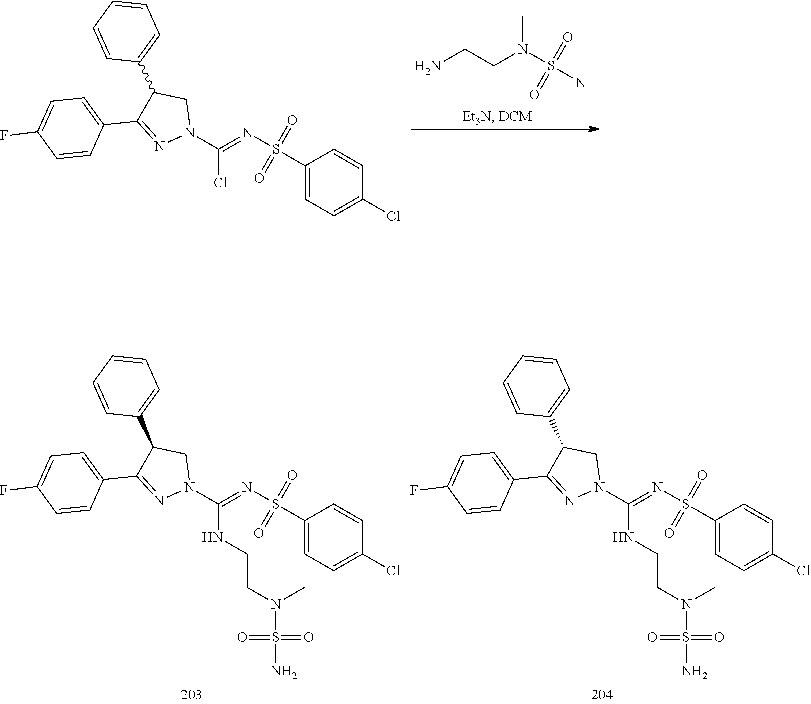

To a stirred solution of 2-(methyl(sulfamoyl)amino)ethyl)amine (121 mg, 0.79 mmol, 1.5 eq.) and triethylamine (0.37 mL, 2.6 mmol, 5.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (250 mg, 0.52 mmol, 1.0 eq.). After stirring at rt for 18 h, solvents were removed under reduced pressure and the crude was chromatographed on silica eluting with ethyl acetate/heptane mixtures (0/100 to 80/20, v/v) to obtain a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—EtOH+0.1% diethylamine, at 35° C., BPR 100 bar. Acquisition frequency was set to 220 nm for the DAD detector.

Compound 203: (39.6 mg, 0.07 mmol, 13% yield, 99% purity, 99% ee, OR: −113.1). 1H NMR (DMSO-d₆, 400 MHz) δ 8.10 (br s, 1H), 7.77-7.85 (m, 4H), 7.53 (d, J=8.6 Hz, 2H), 7.30-7.36 (m, 2H), 7.20-7.28 (m, 5H), 6.76 (s, 2H), 5.08 (dd, J=11.1, 4.2 Hz, 1H), 4.51 (t, J=11.2 Hz, 1H), 4.05 (br dd, J=11.1, 4.2 Hz, 1H), 3.47 (br d, J=5.3 Hz, 2H), 3.10 (br t, J=6.8 Hz, 2H), 2.54 (s, 3H) ppm. LC-MS (ESI+): 593.0 (M+H+).

Compound 204: (35.6 mg, 0.06 mmol, 12% yield, 92% purity, 99% ee, OR: +89.9). 1H NMR (DMSO-d₆, 400 MHz) δ 8.10 (br s, 1H), 7.76-7.84 (m, 4H), 7.53 (d, J=8.5 Hz, 2H), 7.30-7.35 (m, 2H), 7.20-7.27 (m, 5H), 6.76 (s, 2H), 5.08 (dd, J=11.2, 4.1 Hz, 1H), 4.51 (t, J=11.2 Hz, 1H), 4.05 (br dd, J=11.1, 4.0 Hz, 1H), 3.47 (br d, J=5.5 Hz, 2H), 3.10 (br t, J=6.8 Hz, 2H), 2.56 (s, 3H) ppm. LC-MS (ESI+): 593.1 (M+H+).

### Synthesis of (S,E)-N′-((4-chlorophenyl)sulfonyl)-3-(4-fluorophenyl)-N-(2-methyl-2-(sulfamoylamino)propyl)-4-phenyl-4,5-dihydro-1H-pyrazole-1-carboximidamide (Compound 205) & (R,E)-N′-((4-chlorophenyl)sulfonyl)-3-(4-fluorophenyl)-N-(2-methyl-2-(sulfamoylamino)propyl)-4-phenyl-4,5-dihydro-1H-pyrazole-1-carboximidamide (Compound 206)

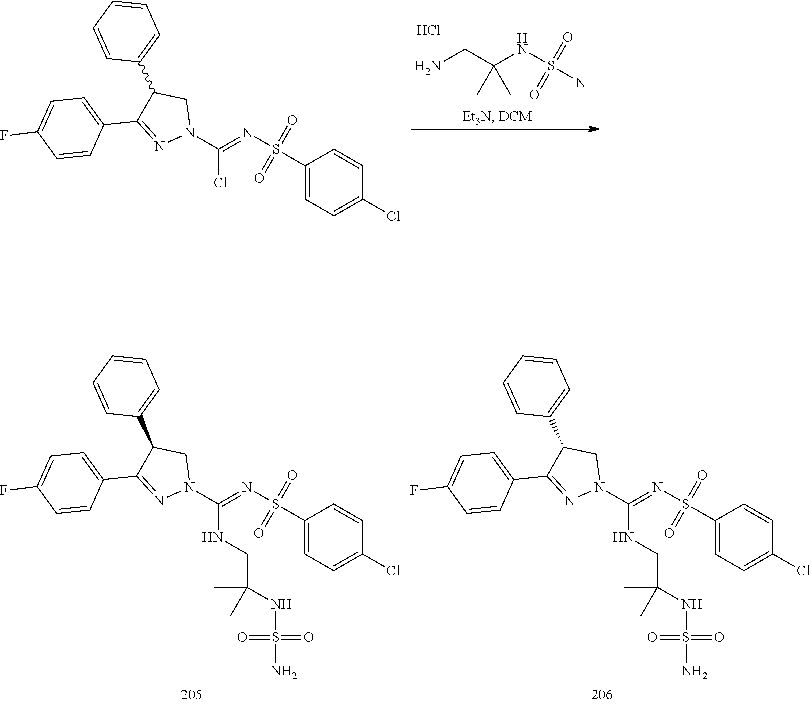

To a stirred solution of 1-Amino-2-(sulfamoylamino)propane hydrochloride (160 mg, 0.79 mmol, 1.5 eq.) and triethylamine (0.37 mL, 2.6 mmol, 5.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (250 mg, 0.52 mmol, 1.0 eq.). After stirring at rt for 18 h, solvents were removed under reduced pressure and the crude was chromatographed on silica eluting with ethyl acetate/heptane mixtures (0/100 to 70/30) to obtain a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 205: (10.6 mg, 0.017 mmol, 3% yield, 99% purity, 99% ee, OR: −62.4). 1H NMR (MeOD-d₄, 400 MHz) δ 7.85 (d, J=8.6 Hz, 2H), 7.76 (dd, J=8.8, 5.4 Hz, 2H), 7.47 (d, J=8.7 Hz, 2H), 7.30-7.36 (m, 2H), 7.27 (d, J=7.2 Hz, 1H), 7.18-7.23 (m, 2H), 7.05 (t, J=8.8 Hz, 2H), 4.94 (dd, J=11.1, 4.6 Hz, 1H), 4.51 (t, J=11.3 Hz, 1H), 4.05 (dd, J=11.3, 4.6 Hz, 1H), 3.71 (br s, 2H), 1.32 (d, J=8.5 Hz, 6H) ppm. LC-MS (ESI+): 607.1 (M+H+); HPLC RT: 3.644 min (Method: VILLA_2T).

Compound 206: (11.1 mg, 0.018 mmol, 3% yield, 96% purity, 99% ee, OR: +118.2). 1H NMR (MeOD-d₄, 400 MHz) δ 7.85 (d, J=8.6 Hz, 2H), 7.76 (dd, J=8.8, 5.4 Hz, 2H), 7.45-7.51 (m, 2H), 7.29-7.36 (m, 2H), 7.27 (d, J=7.2 Hz, 1H), 7.17-7.23 (m, 2H), 7.00-7.09 (m, 2H), 4.94 (dd, J=11.1, 4.6 Hz, 1H), 4.51 (t, J=11.3 Hz, 1H), 4.05 (dd, J=11.3, 4.5 Hz, 1H), 3.71 (br s, 2H), 1.32 (d, J=8.5 Hz, 6H) ppm. LC-MS (ESI+): 606.7 (M+H+); HPLC RT: 3.784 min (Method: VILLA).

### Synthesis of (S,E)-3-(4-chlorophenyl)-N′-((4-chlorophenyl)sulfonyl)-N-(2-methyl-2-(sulfamoylamino)propyl)-4-phenyl-4,5-dihydro-1H-pyrazole-1-carboximidamide (Compound 207) & (R,E)-3-(4-chlorophenyl)-N′-((4-chlorophenyl)sulfonyl)-N-(2-methyl-2-(sulfamoylamino)propyl)-4-phenyl-4,5-dihydro-1H-pyrazole-1-carboximidamide (Compound 208)

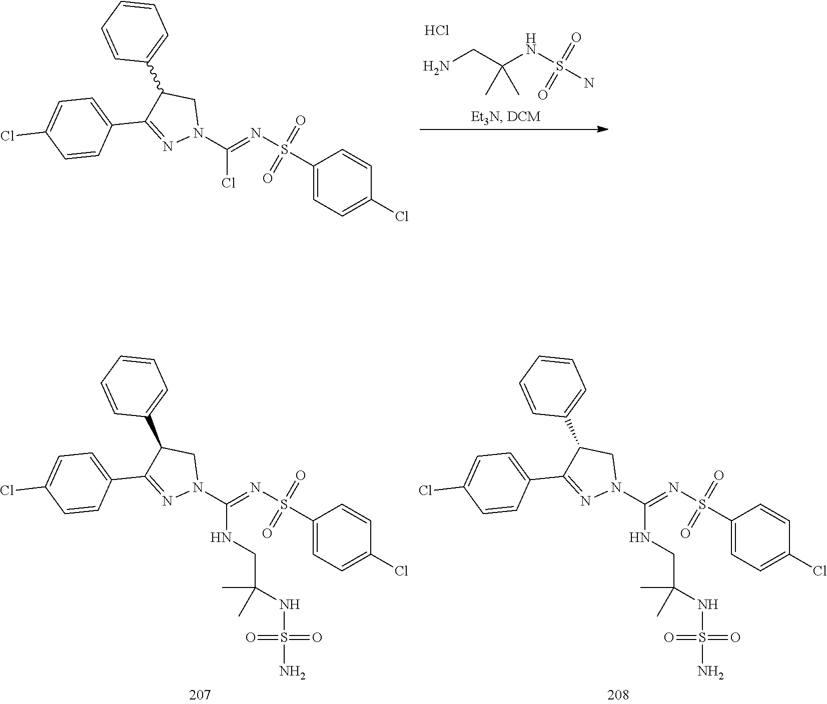

To a stirred solution of 1-Amino-2-(sulfamoylamino)propane hydrochloride (155 mg, 0.76 mmol, 1.5 eq.) and triethylamine (0.35 mL, 2.5 mmol, 5.0 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0 eq.). After stirring at rt for 18 h, solvents were removed under reduced pressure and the crude was chromatographed on silica eluting with ethyl acetate/heptane mixtures (0/100 to 70/30) to obtain a white solid, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 207: (16.2 mg, 0.026 mmol, 5% yield, 99% purity, 99% ee, OR: −90.7). 1H NMR (DMSO-d₆, 400 MHz) δ 7.76-7.83 (m, 3H), 7.70 (d, J=8.6 Hz, 2H), 7.52-7.57 (m, 2H), 7.44 (d, J=8.6 Hz, 2H), 7.31-7.36 (m, 2H), 7.22-7.28 (m, 3H), 6.71 (d, J=7.3 Hz, 3H), 5.09 (br dd, J=11.0, 4.2 Hz, 1H), 4.53 (t, J=11.3 Hz, 1H), 4.04 (br dd, J=10.6, 3.4 Hz, 1H), 3.45-3.59 (m, 2H), 1.18 (d, J=1.8 Hz, 6H) ppm. LC-MS (ESI+): 622.7 (M+H+).

Compound 208: (14.4 mg, 0.023 mmol, 4% yield, 99% purity, 99% ee, OR: +79.4). 1H NMR (DMSO-d₆, 400 MHz) δ 7.76-7.84 (m, 3H), 7.70 (d, J=8.6 Hz, 2H), 7.53-7.57 (m, 2H), 7.44 (d, J=8.7 Hz, 2H), 7.31-7.37 (m, 2H), 7.22-7.28 (m, 3H), 6.71 (d, J=7.3 Hz, 3H), 5.09 (br dd, J=11.1, 4.2 Hz, 1H), 4.53 (t, J=11.3 Hz, 1H), 4.04 (br dd, J=10.9, 3.7 Hz, 1H), 3.52 (br s, 2H), 1.18 (d, J=1.9 Hz, 6H) ppm. LC-MS (ESI+): 622.7 (M+H+).

### Synthesis of (S,E)-3-(4-chlorophenyl)-N′-((4-chlorophenyl)sulfonyl)-N-((S)-3-methoxy-2-(sulfamoylamino)propyl)-4-phenyl-4,5-dihydro-1H-pyrazole-1-carboximidamide (Compound 209) & (R,E)-3-(4-chlorophenyl)-N′-((4-chlorophenyl)sulfonyl)-N-((S)-3-methoxy-2-(sulfamoylamino)propyl)-4-phenyl-4,5-dihydro-1H-pyrazole-1-carboximidamide (Compound 210)

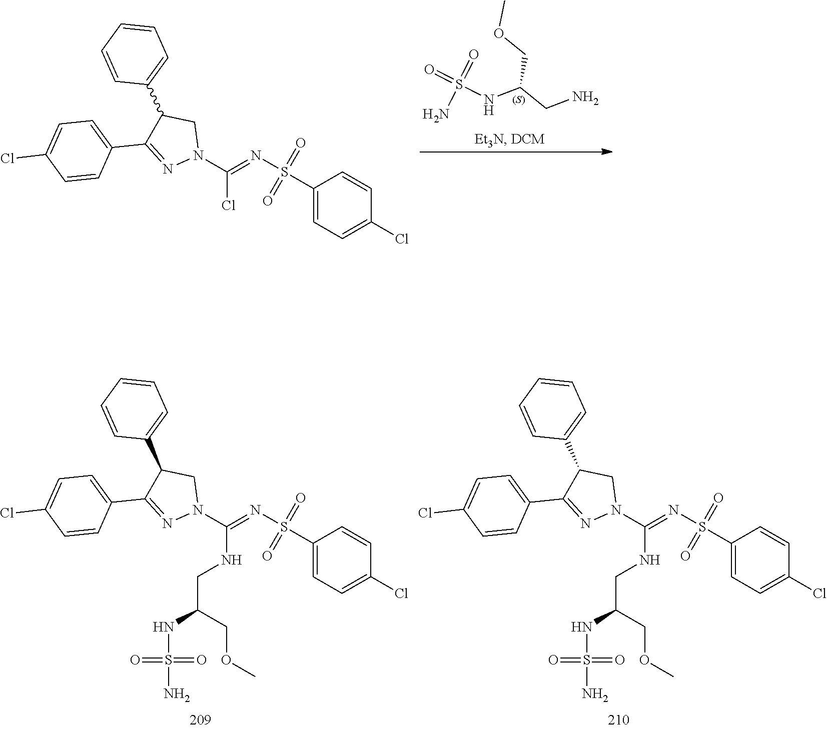

To a stirred solution of (S)-(1-Amino-3-methoxypropan-2-yl)sulfamide (150 mg, 0.81 mmol, 1.5 eq.) and triethylamine (0.17 mL, 1.22 mmol, 3.0 eq.) in dichloromethane (5 mL) was added (Z)-N-((4-chlorophenyl)sulfonyl)-3-(4-chlorophenyl)-4-phenyl-4,5-dihydro-1H-pyrazole-1-carbimidoyl chloride (250 mg, 0.52 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, and concentrated in vacuo. The residue was purified by flash chromatography on silica eluting with ethyl acetate/heptane mixtures (0/100 to 60/40, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 209: (8.9 mg, 0.014 mmol, 3% yield, 96% purity, 99% ee, OR: −99.9). 1H NMR (MeOD-d₄, 400 MHz) δ 7.86 (d, J=8.6 Hz, 2H), 7.70 (d, J=8.7 Hz, 2H), 7.47 (d, J=8.6 Hz, 2H), 7.18-7.34 (m, 7H), 4.91 (dd, J=11.2, 4.5 Hz, 1H), 4.51 (t, J=11.3 Hz, 1H), 4.00-4.09 (m, 1H), 3.71-3.85 (m, 2H), 3.44-3.56 (m, 3H), 3.36 (s, 3H) ppm. LC-MS (ESI+): 640.1 (M+H+).

Compound 210: (32.6 mg, 0.051 mmol, 13% yield, 98% purity, 99% ee, OR: +45.8). 1H NMR (DMSO-d₆, 400 MHz) δ 7.79-7.88 (m, 3H), 7.73 (d, J=8.6 Hz, 2H), 7.54 (d, J=8.6 Hz, 2H), 7.44 (d, J=8.6 Hz, 2H), 7.20-7.37 (m, 5H), 6.81 (br d, J=7.2 Hz, 1H), 6.65 (s, 2H), 5.07 (br dd, J=11.1, 4.1 Hz, 1H), 4.53 (t, J=11.2 Hz, 1H), 4.34 (d, J=4.3 Hz, 1H), 4.03 (br dd, J=10.9, 3.7 Hz, 1H), 3.60 (br d, J=7.2 Hz, 2H), 3.42 (br dd, J=9.5, 3.9 Hz, 1H), 3.27-3.30 (m, 1H), 3.23 (s, 3H) ppm. LC-MS (ESI+): 640.0 (M+H+).

### Synthesis of (S,E)-N′-((4-chlorophenyl)sulfonyl)-3-(4-fluorophenyl)-N-((S)-3-methyl-2-(sulfamoylamino)butyl)-4-phenyl-4,5-dihydro-1H-pyrazole-1-carboximidamide (Compound 211) & (R,E)-N′-((4-chlorophenyl)sulfonyl)-3-(4-fluorophenyl)-N-((S)-3-methyl-2-(sulfamoylamino)butyl)-4-phenyl-4,5-dihydro-1H-pyrazole-1-carboximidamide (Compound 212)

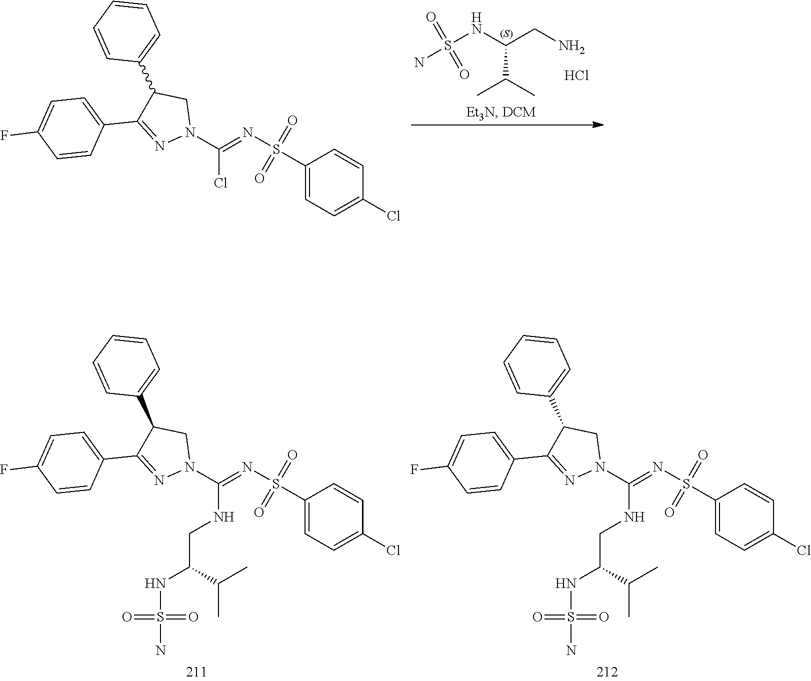

To a stirred solution of (S)-(1-amino-3-methylbutan-2-yl)sulfamide hydrochloride (274 mg, 1.26 mmol, 2 eq.) and triethylamine (0.26 mL, 1.89 mmol, 3.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (300 mg, 0.63 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 211: (32.2 mg, 0.052 mmol, 8% yield, 99% purity, 99% ee, OR: −8.6). 1H NMR (DMSO-d₆, 400 MHz) δ 7.75-7.88 (m, 5H), 7.53 (dd, J=8.5, 2.8 Hz, 2H), 7.29-7.36 (m, 2H), 7.18-7.28 (m, 5H), 6.77 (br d, J=7.5 Hz, 1H), 6.58 (d, J=4.6 Hz, 2H), 5.08 (dd, J=11.0, 3.9 Hz, 1H), 4.46-4.56 (m, 1H), 3.99-4.08 (m, 1H), 3.48-3.63 (m, 1H), 3.25 (br d, J=3.6 Hz, 2H), 1.67-1.77 (m, 1H), 0.77-0.83 (m, 6H) ppm. LC-MS (ESI+): 621.1 (M+H+).

Compound 212: (85.2 mg, 0.14 mmol, 22% yield, 99% purity, 99% ee, OR: +80.8). 1H NMR (DMSO-d₆, 400 MHz) δ 7.75-7.89 (m, 5H), 7.53 (d, J=8.6 Hz, 2H), 7.17-7.38 (m, 7H), 6.77 (br d, J=7.2 Hz, 1H), 6.57 (s, 2H), 5.08 (dd, J=11.1, 4.1 Hz, 1H), 4.50 (t, J=11.1 Hz, 1H), 4.05 (dd, J=11.1, 4.1 Hz, 1H), 3.46-3.60 (m, 1H), 3.25 (br d, J=7.1 Hz, 2H), 1.66-1.76 (m, 1H), 0.79 (dd, J=15.5, 6.8 Hz, 6H) ppm. LC-MS (ESI+): 621.1 (M+H+).

### Synthesis of (S,E)-N′-((4-chlorophenyl)sulfonyl)-3-(4-fluorophenyl)-4-phenyl-N-((R)-2-(sulfamoylamino)propyl)-4,5-dihydro-1H-pyrazole-1-carboximidamide (Compound 213) & (R,E)-N′-((4-chlorophenyl)sulfonyl)-3-(4-fluorophenyl)-4-phenyl-N-((R)-2-(sulfamoylamino)propyl)-4,5-dihydro-1H-pyrazole-1-carboximidamide (Compound 214)

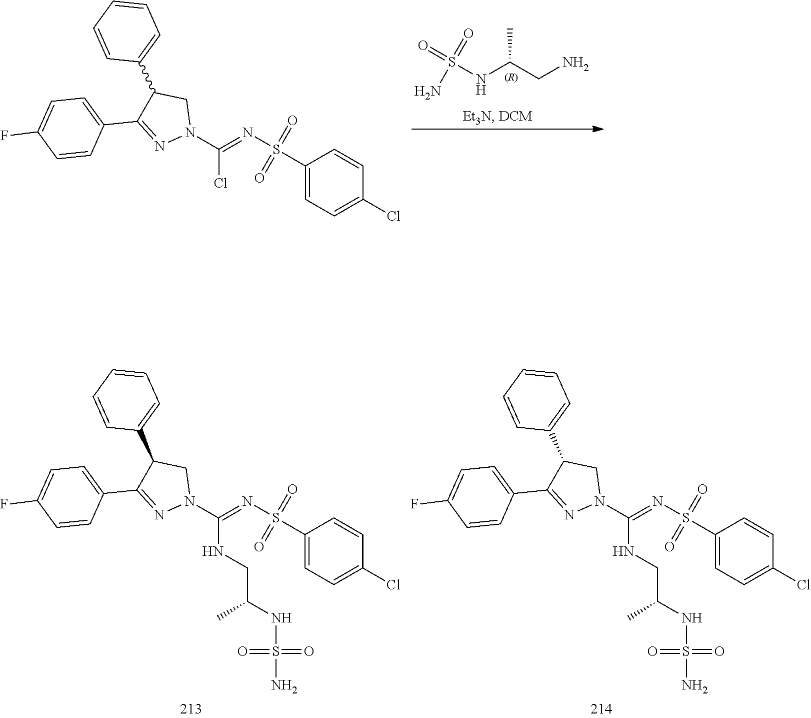

To a stirred solution of (R)-(1-Aminopropan-2-yl)sulfamide hydrochloride (110 mg, 0.58 mmol, 1.1 eq.) and triethylamine (0.22 mL, 1.57 mmol, 3.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (250 mg, 0.52 mmol, 1.0 eq.). After stirring at rt for 18 h, solvents were removed under reduced pressure and the crude was chromatographed on silica eluting with ethyl acetate/heptane mixtures (0/100 to 80/20, v/v) to obtain a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 213: (72.7 mg, 0.12 mmol, 23% yield, 97% purity, 99% ee, OR: −115.4). 1H NMR (DMSO-d₆, 400 MHz) δ 7.82 (br t, J=4.7 Hz, 1H), 7.70-7.78 (m, 4H), 7.47 (d, J=8.6 Hz, 2H), 7.23-7.31 (m, 2H), 7.13-7.22 (m, 5H), 6.70 (br d, J=7.2 Hz, 1H), 6.53 (br s, 2H), 5.01 (dd, J=11.1, 4.2 Hz, 1H), 4.44 (t, J=11.2 Hz, 1H), 3.95 (br dd, J=11.2, 4.1 Hz, 1H), 3.46 (dt, J=12.4, 6.3 Hz, 1H), 3.36-3.41 (m, 1H), 3.21-3.28 (m, 1H), 1.01 (d, J=6.6 Hz, 3H) ppm. LC-MS (ESI+): 594.0 (M+H+).

Compound 214: (84.1 mg, 0.14 mmol, 27% yield, 99% purity, 99% ee, OR: +128.8). 1H NMR (DMSO-d₆, 400 MHz) δ 7.87 (br s, 1H), 7.81 (d, J=8.6 Hz, 2H), 7.72-7.80 (m, 2H), 7.54 (d, J=8.6 Hz, 2H), 7.29-7.37 (m, 2H), 7.16-7.28 (m, 5H), 6.75 (br d, J=7.5 Hz, 1H), 6.59 (br s, 2H), 5.07 (dd, J=11.2, 4.2 Hz, 1H), 4.50 (t, J=11.2 Hz, 1H), 4.04 (br dd, J=11.3, 4.1 Hz, 1H), 3.48-3.58 (m, 1H), 3.43 (br dd, J=12.3, 1.6 Hz, 1H), 3.24 (br s, 1H), 1.08 (d, J=6.5 Hz, 3H) ppm. LC-MS (ESI+): 594.0 (M+H+)

### Synthesis of (S,E)-3-(4-chlorophenyl)-N′-((4-chlorophenyl)sulfonyl)-4-phenyl-N-((R)-2-(sulfamoylamino)propyl)-4,5-dihydro-1H-pyrazole-1-carboximidamide (Compound 215) & (R,E)-3-(4-chlorophenyl)-N′-((4-chlorophenyl)sulfonyl)-4-phenyl-N-((R)-2-(sulfamoylamino)propyl)-4,5-dihydro-1H-pyrazole-1-carboximidamide (Compound 216)

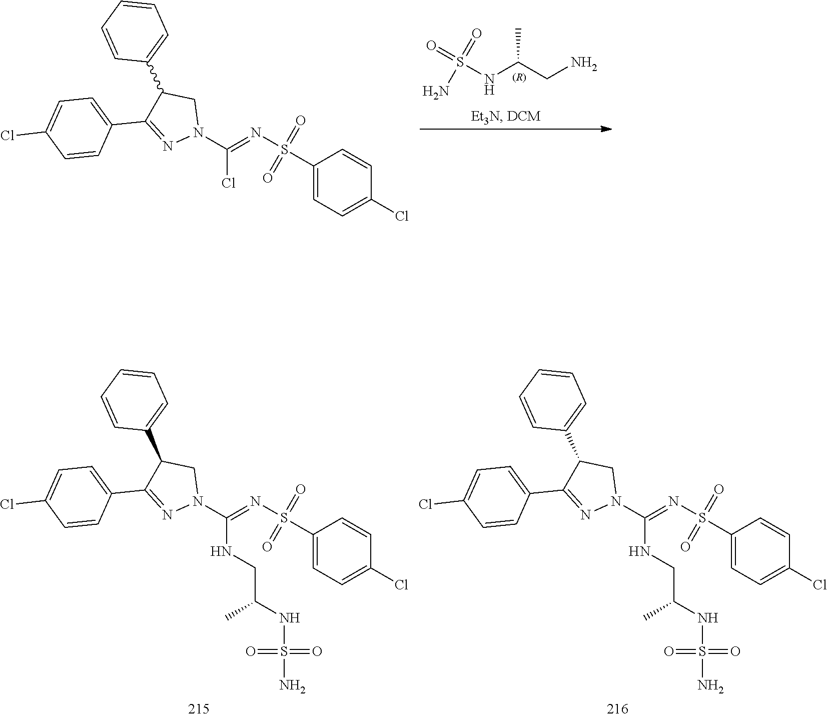

To a stirred solution of (R)-(1-Aminopropan-2-yl)sulfamide hydrochloride (127 mg, 0.67 mmol, 1.1 eq.) and triethylamine (0.26 mL, 1.83 mmol, 3.0 eq.) in dichloromethane (5 mL) was added (Z)-N-((4-chlorophenyl)sulfonyl)-3-(4-chlorophenyl)-4-phenyl-4,5-dihydro-1H-pyrazole-1-carbimidoyl chloride (300 mg, 0.61 mmol, 1.0 eq.). After stirring at rt for 18 h, solvents were removed under reduced pressure and the crude was chromatographed on silica eluting with ethyl acetate/heptane mixtures (0/100 to 80/20, v/v) to obtain a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 215: (116 mg, 0.19 mmol, 31% yield, 98% purity, 99% ee, OR: −89.5). 1H NMR (DMSO-d₆, 400 MHz) δ 7.87-7.94 (m, 1H), 7.81 (d, J=8.6 Hz, 2H), 7.69-7.78 (m, 2H), 7.50-7.57 (m, 2H), 7.44 (d, J=8.6 Hz, 2H), 7.29-7.36 (m, 2H), 7.19-7.29 (m, 3H), 6.75 (d, J=7.7 Hz, 1H), 6.58 (s, 2H), 5.07 (dd, J=11.1, 4.1 Hz, 1H), 4.52 (t, J=11.2 Hz, 1H), 4.02 (br dd, J=11.1, 4.1 Hz, 1H), 3.52 (dt, J=13.3, 6.7 Hz, 1H), 3.38-3.46 (m, 1H), 3.24-3.30 (m, 1H), 1.07 (d, J=6.6 Hz, 3H) ppm. LC-MS (ESI+): 609.0 (M+H+).

Compound 216: (107.4 mg, 0.18 mmol, 29% yield, 99% purity, 99% ee, OR: +101.1). 1H NMR (DMSO-d₆, 400 MHz) δ 7.90 (br s, 1H), 7.81 (d, J=8.5 Hz, 2H), 7.73 (d, J=8.5 Hz, 2H), 7.54 (d, J=8.5 Hz, 2H), 7.44 (d, J=8.6 Hz, 2H), 7.29-7.35 (m, 2H), 7.21-7.28 (m, 3H), 6.75 (br d, J=7.2 Hz, 1H), 6.59 (br s, 2H), 5.07 (dd, J=11.1, 4.2 Hz, 1H), 4.51 (t, J=11.2 Hz, 1H), 4.05 (br dd, J=11.0, 4.1 Hz, 1H), 3.48-3.59 (m, 1H), 3.43 (br d, J=9.3 Hz, 1H), 3.21-3.28 (m, 1H), 1.08 (d, J=6.5 Hz, 3H) ppm. LC-MS (ESI+): 609.0 (M+H+).

### Synthesis of (S,E)-3-(4-chlorophenyl)-N′-((4-chlorophenyl)sulfonyl)-N-((R)-3-methyl-2-(sulfamoylamino)butyl)-4-phenyl-4,5-dihydro-1H-pyrazole-1-carboximidamide (Compound 217) & (R,E)-3-(4-chlorophenyl)-N′-((4-chlorophenyl)sulfonyl)-N-((R)-3-methyl-2-(sulfamoylamino)butyl)-4-phenyl-4,5-dihydro-1H-pyrazole-1-carboximidamide (Compound 218)

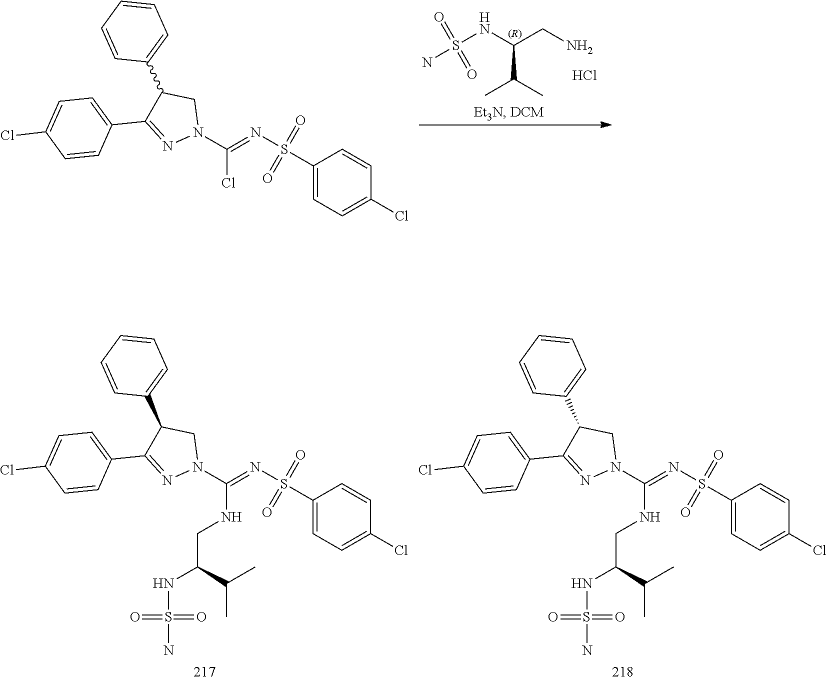

To a stirred solution of (R)-(1-amino-3-methylbutan-2-yl)sulfamide hydrochloride (146 mg, 0.67 mmol, 1.10 eq.) and triethylamine (0.26 mL, 1.83 mmol, 3.0 eq.) in dichloromethane (5 mL) was added (Z)-N-((4-chlorophenyl)sulfonyl)-3-(4-chlorophenyl)-4-phenyl-4,5-dihydro-1H-pyrazole-1-carbimidoyl chloride (300 mg, 0.63 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 217: (20.8 mg, 0.033 mmol, 5% yield, 95% purity, 99% ee, OR: −92.9). 1H NMR (DMSO-d₆, 400 MHz) δ 7.87 (br s, 1H), 7.80 (d, J=8.6 Hz, 2H), 7.74 (d, J=8.6 Hz, 2H), 7.53 (d, J=8.6 Hz, 2H), 7.44 (d, J=8.7 Hz, 2H), 7.32 (d, J=7.5 Hz, 2H), 7.19-7.28 (m, 3H), 6.76 (br d, J=7.8 Hz, 1H), 6.57 (s, 2H), 5.08 (dd, J=11.2, 4.3 Hz, 1H), 4.51 (t, J=11.2 Hz, 1H), 4.05 (br dd, J=11.1, 4.2 Hz, 1H), 3.54 (br s, 1H), 3.24-3.27 (m, 2H), 1.64-1.78 (m, 1H), 0.79 (dd, J=16.2, 6.8 Hz, 6H) ppm. LC-MS (ESI+): 639.0 (M+H+).

Compound 218: (20.0 mg, 0.031 mmol, 5% yield, 99% purity, 99% ee, OR: +81.6). 1H NMR (DMSO-d₆, 400 MHz) δ 7.85 (br s, 1H), 7.81 (d, J=8.6 Hz, 2H), 7.73 (br d, J=8.3 Hz, 2H), 7.54 (d, J=8.6 Hz, 2H), 7.44 (d, J=8.6 Hz, 2H), 7.30-7.36 (m, 2H), 7.20-7.28 (m, 3H), 6.76 (br d, J=6.9 Hz, 1H), 6.58 (br s, 2H), 5.08 (br dd, J=11.3, 3.9 Hz, 1H), 4.53 (br t, J=11.2 Hz, 1H), 3.96-4.09 (m, 1H), 3.54 (br dd, J=3.5, 1.7 Hz, 1H), 3.23 (br s, 2H), 1.72 (br dd, J=11.3, 6.7 Hz, 1H), 0.80 (t, J=7.1 Hz, 6H) ppm. LC-MS (ESI+): 639.0 (M+H+).

### Synthesis of (S,E)-3-(4-chlorophenyl)-N′-((4-chlorophenyl)sulfonyl)-N-((S)-3-methyl-2-(sulfamoylamino)butyl)-4-phenyl-4,5-dihydro-1H-pyrazole-1-carboximidamide (Compound 219) & (R,E)-3-(4-chlorophenyl)-N′-((4-chlorophenyl)sulfonyl)-N-((S)-3-methyl-2-(sulfamoylamino)butyl)-4-phenyl-4,5-dihydro-1H-pyrazole-1-carboximidamide (Compound 220)

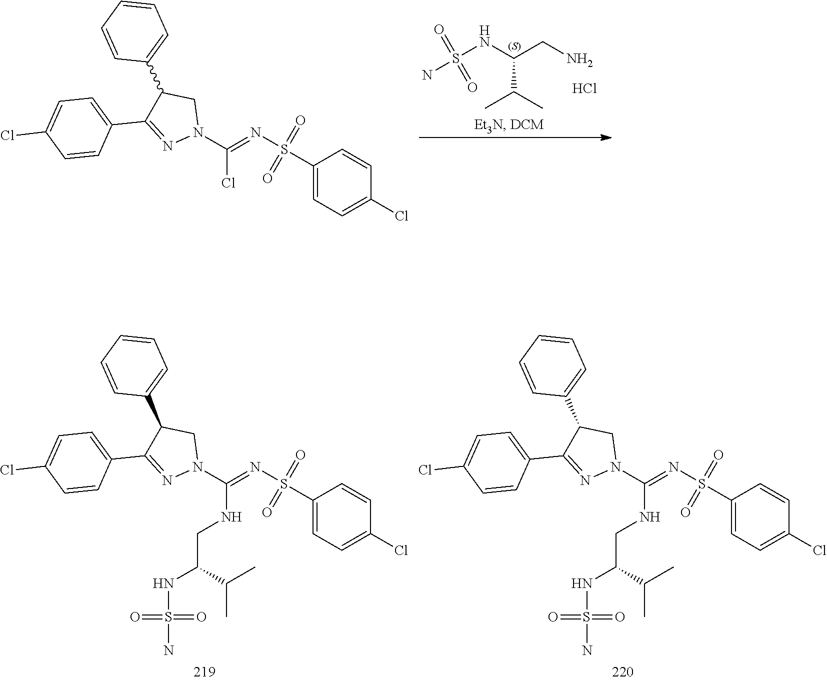

To a stirred solution of (S)-(1-amino-3-methylbutan-2-yl)sulfamide hydrochloride (265 mg, 1.22 mmol, 2 eq.) and triethylamine (0.26 mL, 1.89 mmol, 3.0 eq.) in dichloromethane (5 mL) was added (Z)-N-((4-chlorophenyl)sulfonyl)-3-(4-chlorophenyl)-4-phenyl-4,5-dihydro-1H-pyrazole-1-carbimidoyl chloride (300 mg, 0.63 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two diastereomers as white solids.
SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 219: (11.0 mg, 0.017 mmol, 3% yield, 98% purity, 99% ee, OR: +115.8). 1H NMR (DMSO-d₆, 400 MHz) δ 7.73 (d, J=8.5 Hz, 2H), 7.66 (d, J=8.6 Hz, 2H), 7.48 (d, J=8.6 Hz, 3H), 7.38 (d, J=8.6 Hz, 2H), 7.23-7.30 (m, 2H), 7.19 (br t, J=6.8 Hz, 3H), 6.58 (br s, 1H), 6.54 (s, 2H), 4.93-5.10 (m, 1H), 4.51-4.74 (m, 1H), 3.79-4.00 (m, 1H), 3.55-3.76 (m, 1H), 3.06 (br s, 3H), 0.72 (br d, J=6.6 Hz, 6H) ppm. LC-MS (ESI+): 637.0 (M+H+).

Compound 220: (7.8 mg, 0.012 mmol, 2% yield, 99% purity, 99% ee, OR: −13.6). 1H NMR (DMSO-d₆, 400 MHz) δ 7.70-7.89 (m, 5H), 7.54 (d, J=8.4 Hz, 2H), 7.44 (d, J=8.5 Hz, 2H), 7.22-7.36 (m, 5H), 6.76 (br d, J=7.2 Hz, 1H), 6.58 (s, 2H), 5.04-5.12 (m, 1H), 4.48-4.59 (m, 1H), 4.04 (br dd, J=11.3, 4.2 Hz, 1H), 3.47-3.60 (m, 1H), 3.25 (br dd, J=5.0, 2.1 Hz, 3H), 0.80 (t, J=7.1 Hz, 6H) ppm. LC-MS (ESI+): 637.0 (M+H+).

### Synthesis of (S,E)-3-(4-chlorophenyl)-N-((S)-3-methyl-2-(sulfamoylamino)butyl)-4-phenyl-N′-((4-(trifluoromethyl)phenyl)sulfonyl)-4,5-dihydro-1H-pyrazole-1-carboximidamide (Compound 221) & (R,E)-3-(4-chlorophenyl)-N-((S)-3-methyl-2-(sulfamoylamino)butyl)-4-phenyl-N′-((4-(trifluoromethyl)phenyl)sulfonyl)-4,5-dihydro-1H-pyrazole-1-carboximidamide (Compound 222)

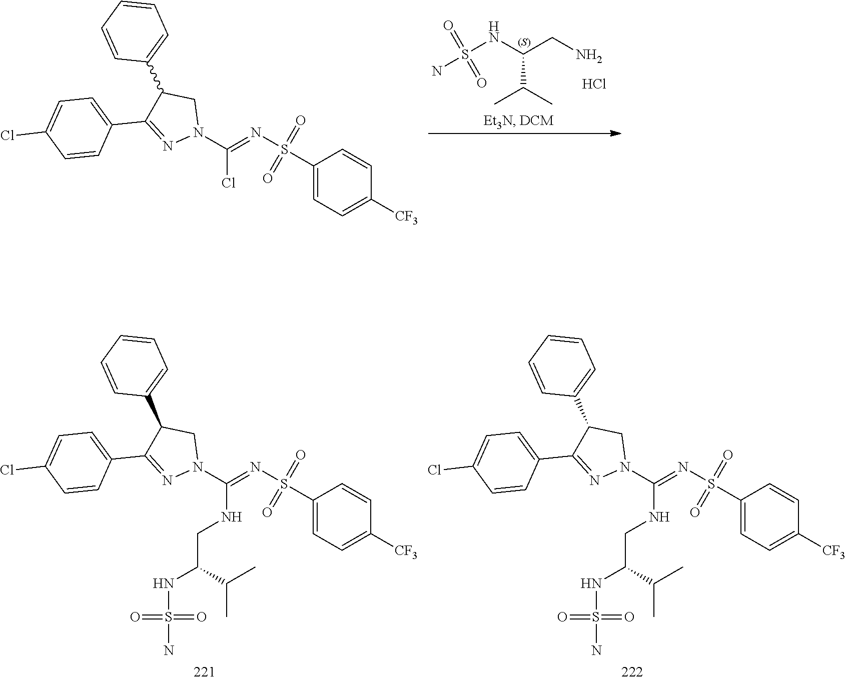

To a stirred solution of (S)-(1-amino-3-methylbutan-2-yl)sulfamide hydrochloride (136 mg, 0.63 mmol, 2 eq.) and triethylamine (0.13 mL, 0.94 mmol, 3.0 eq.) in dichloromethane (5 mL) was added (Z)-3-(4-chlorophenyl)-4-phenyl-N-((4-(trifluoromethyl)phenyl)sulfonyl)-4,5-dihydro-1H-pyrazole-1-carbimidoyl chloride (165 mg, 0.31 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 60/40, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 221: (17.0 mg, 0.025 mmol, 8% yield, 99% purity, 99% ee, OR: −10.0). 1H NMR (DMSO-d₆, 400 MHz) δ 7.95 (br d, J=8.2 Hz, 2H), 7.79 (br d, J=8.1 Hz, 2H), 7.60-7.69 (m, 2H), 7.37 (br d, J=8.6 Hz, 2H), 7.14-7.30 (m, 6H), 6.68 (br d, J=7.5 Hz, 1H), 6.51-6.55 (m, 2H), 5.02 (br dd, J=11.3, 4.3 Hz, 1H), 4.48 (t, J=11.1 Hz, 1H), 3.98 (br dd, J=11.2, 4.3 Hz, 1H), 3.38-3.49 (m, 1H), 3.15 (br d, J=5.1 Hz, 2H), 3.00-3.10 (m, 1H), 0.70 (br t, J=7.0 Hz, 6H) ppm. LC-MS (ESI+): 671.0 (M+H+).

Compound 222: (1.4 mg, 0.002 mmol, 1% yield, 98% purity, 99% ee, OR: +211.6). 1H NMR (MeOD-d₄, 400 MHz) δ 8.07 (d, J=8.2 Hz, 2H), 7.67-7.82 (m, 4H), 7.18-7.36 (m, 7H), 4.93 (dd, J=11.2, 4.6 Hz, 1H), 4.54 (t, J=11.2 Hz, 1H), 4.03 (dd, J=11.2, 4.6 Hz, 1H), 3.62-3.71 (m, 1H), 3.35-3.43 (m, 2H), 1.74-1.86 (m, 1H), 0.91 (dd, J=6.8, 3.1 Hz, 6H) ppm. LC-MS (ESI+): 671.0 (M+H+).

### Synthesis of (S,E)-N′-((4-chlorophenyl)sulfonyl)-3-(4-fluorophenyl)-N-((S)-3-methoxy-2-(sulfamoylamino)propyl)-4-phenyl-4,5-dihydro-1H-pyrazole-1-carboximidamide (Compound 223) & (R,E)-N′-((4-chlorophenyl)sulfonyl)-3-(4-fluorophenyl)-N-((S)-3-methoxy-2-(sulfamoylamino)propyl)-4-phenyl-4,5-dihydro-1H-pyrazole-1-carboximidamide (Compound 224)

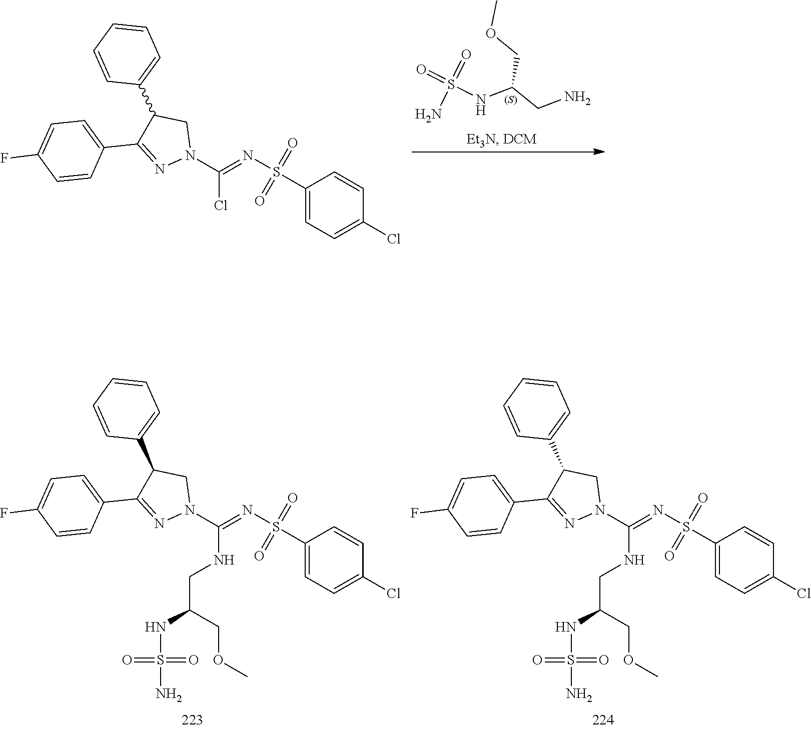

To a stirred solution of (S)-(1-Amino-3-methoxypropan-2-yl)sulfamide (154 mg, 0.84 mmol, 2.0 eq.) and triethylamine (0.18 mL, 1.26 mmol, 3.0 eq.) in dichloromethane (5 mL) was added (Z)-N-((4-chlorophenyl)sulfonyl)-3-(4-fluorophenyl)-4-phenyl-4,5-dihydro-1H-pyrazole-1-carbimidoyl chloride (200 mg, 0.42 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 60/40, v/v) to give a colorless oil, which was further subjected to SFC purification to resolve the diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 223: (29.6 mg, 0.05 mmol, 11% yield, 99% purity, 99% ee, OR: −51.3). 1H NMR (DMSO-d₆, 400 MHz) δ 7.74-7.88 (m, 5H), 7.53 (d, J=8.5 Hz, 2H), 7.30-7.36 (m, 2H), 7.19-7.28 (m, 5H), 6.81 (d, J=7.4 Hz, 1H), 6.65 (s, 2H), 5.08 (dd, J=11.1, 4.2 Hz, 1H), 4.49 (t, J=11.1 Hz, 1H), 4.05 (br dd, J=11.1, 3.9 Hz, 1H), 3.60 (br d, J=7.0 Hz, 2H), 3.38-3.44 (m, 1H), 3.28 (br s, 2H), 3.22 (s, 3H) ppm. LC-MS (ESI+): 623.1 (M+H+).

Compound 224: (6 mg, 99% purity, 99% ee, OR: +52.3). 1H NMR (DMSO-d₆, 400 MHz) δ 7.74-7.88 (m, 5H), 7.53 (d, J=8.5 Hz, 2H), 7.30-7.36 (m, 2H), 7.19-7.28 (m, 5H), 6.81 (d, J=7.4 Hz, 1H), 6.65 (s, 2H), 5.08 (dd, J=11.1, 4.2 Hz, 1H), 4.49 (t, J=11.1 Hz, 1H), 4.05 (br dd, J=11.1, 3.9 Hz, 1H), 3.60 (br d, J=7.0 Hz, 2H), 3.38-3.44 (m, 1H), 3.28 (br s, 2H), 3.22 (s, 3H) ppm. LC-MS (ESI+): 623.1 (M+H+).

### Synthesis of (1R,3S)-3-((S,E)-3-(4-chlorophenyl)-N′-((4-chlorophenyl)sulfonyl)-4-phenyl-4,5-dihydro-1H-pyrazole-1-carboximidamido)cyclobutyl sulfamate (Compound 225) & (1R,3S)-3-((R,E)-3-(4-chlorophenyl)-N′-((4-chlorophenyl)sulfonyl)-4-phenyl-4,5-dihydro-1H-pyrazole-1-carboximidamido)cyclobutyl sulfamate (Compound 226)

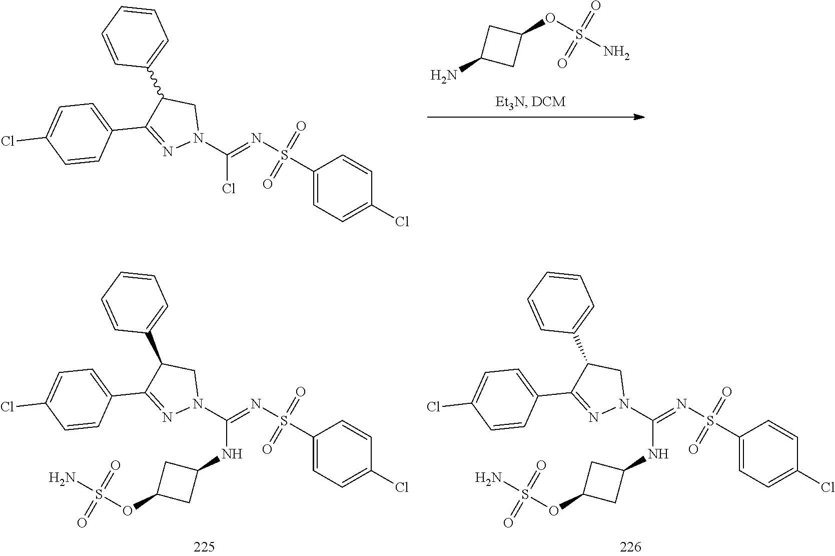 [SMILES: O=S(=O)(/N=C(\Cl)N1CC(c2ccccc2)C(c2ccc(Cl)cc2)=N1)c1ccc(Cl)cc1 ; NS(=O)(=O)O[C@H]1C[C@@H](N)C1 ; NS(=O)(=O)O[C@H]1C[C@@H](N/C(=N\S(=O)(=O)c2ccc(Cl)cc2)N2C[C@H](c3ccccc3)C(c3ccc(Cl)cc3)=N2)C1 ; NS(=O)(=O)O[C@H]1C[C@@H](N/C(=N\S(=O)(=O)c2ccc(Cl)cc2)N2C[C@@H](c3ccccc3)C(c3ccc(Cl)cc3)=N2)C1]

To a stirred solution of Sulfamic acid 3-amino-cyclobutyl ester (91 mg, 0.55 mmol, 2 eq.) and triethylamine (0.12 mL, 0.82 mmol, 3.0 eq.) in dichloromethane (5 mL) was added (E)-3-(4-chlorophenyl)-N-((4-chlorophenyl)sulfonyl)-4-phenyl-4,5-dihydro-1H-pyrazole-1-carbimidoyl chloride (135 mg, 0.27 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 60/40, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 225: (39.2 mg, 0.063 mmol, 23% yield, 99% purity, 99% ee, OR: +53.2). 1H NMR (CDCl₃, 400 MHz) δ 7.84 (d, J=8.6 Hz, 2H), 7.51 (d, J=8.6 Hz, 2H), 7.41 (d, J=8.6 Hz, 2H), 7.27-7.36 (m, 5H), 7.11 (d, J=6.8 Hz, 2H), 4.76 (quin, J=7.0 Hz, 1H), 4.65 (dd, J=11.4, 5.6 Hz, 1H), 4.43-4.54 (m, 1H), 4.34 (dq, J=15.0, 7.6 Hz, 1H), 3.98-4.08 (m, 1H), 3.00-3.13 (m, 2H), 2.36-2.48 (m, 2H) ppm. LC-MS (ESI+): 622.0 (M+H+).

Compound 226: (40.6 mg, 0.065 mmol, 24% yield, 99% purity, 99% ee, OR: −42.0). 1H NMR (CDCl₃, 400 MHz) δ 7.84 (d, J=8.6 Hz, 2H), 7.51 (d, J=8.6 Hz, 2H), 7.40 (d, J=8.5 Hz, 2H), 7.28-7.35 (m, 4H), 7.24-7.27 (m, 1H), 7.08-7.13 (m, 2H), 4.76 (quin, J=7.0 Hz, 1H), 4.65 (dd, J=11.4, 5.5 Hz, 1H), 4.49 (t, J=11.6 Hz, 1H), 4.28-4.40 (m, 1H), 4.04 (dd, J=11.6, 5.4 Hz, 1H), 3.00-3.13 (m, 2H), 2.35-2.48 (m, 2H) ppm. LC-MS (ESI+): 622.0 (M+H+)

### Synthesis of (1R,3s)-3-((S,E)-3-(4-chlorophenyl)-4-phenyl-N′-((4-(trifluoromethyl)phenyl)sulfonyl)-4,5-dihydro-1H-pyrazole-1-carboximidamido)cyclobutyl sulfamate (Compound 227) & (1R,3s)-3-((R,E)-3-(4-chlorophenyl)-4-phenyl-N′-((4-(trifluoromethyl)phenyl)sulfonyl)-4,5-dihydro-1H-pyrazole-1-carboximidamido)cyclobutyl sulfamate (Compound 228)

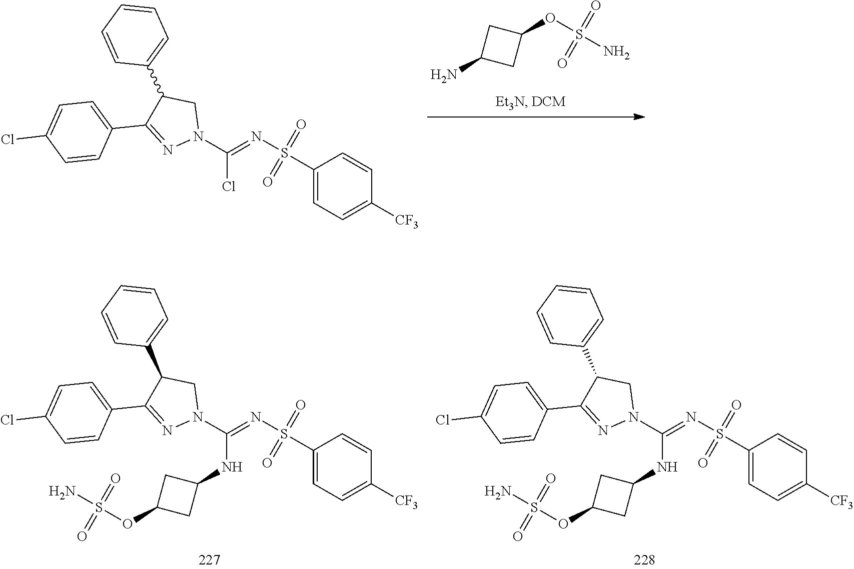 [SMILES: O=S(=O)(/N=C(\Cl)N1CC(c2ccccc2)C(c2ccc(Cl)cc2)=N1)c1ccc(C(F)(F)F)cc1 ; NS(=O)(=O)O[C@H]1C[C@@H](N)C1 ; NS(=O)(=O)O[C@H]1C[C@@H](N/C(=N\S(=O)(=O)c2ccc(C(F)(F)F)cc2)N2C[C@H](c3ccccc3)C(c3ccc(Cl)cc3)=N2)C1 ; NS(=O)(=O)O[C@H]1C[C@@H](N/C(=N\S(=O)(=O)c2ccc(C(F)(F)F)cc2)N2C[C@@H](c3ccccc3)C(c3ccc(Cl)cc3)=N2)C1]

To a stirred solution of sulfamic acid 3-amino-cyclobutyl ester (126 mg, 0.76 mmol, 2 eq.) and triethylamine (0.16 mL, 1.14 mmol, 3.0 eq.) in dichloromethane (5 mL) was added (Z)-3-(4-chlorophenyl)-4-phenyl-N-((4-(trifluoromethyl)phenyl)sulfonyl)-4,5-dihydro-1H-pyrazole-1-carbimidoyl chloride (200 mg, 0.38 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 60/40, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 227: (48.7 mg, 0.074 mmol, 20% yield, 99% purity, 99% ee, OR: +57.9). 1H NMR (DMSO-d₆, 400 MHz) δ 8.25-8.43 (m, 1H), 7.98 (d, J=8.1 Hz, 2H), 7.85 (d, J=8.3 Hz, 2H), 7.75 (d, J=8.6 Hz, 2H), 7.41-7.52 (m, 4H), 7.29-7.36 (m, 2H), 7.17-7.28 (m, 3H), 5.05 (dd, J=11.1, 4.3 Hz, 1H), 4.41-4.55 (m, 2H), 3.98-4.11 (m, 1H), 3.90 (dd, J=11.2, 4.2 Hz, 1H), 2.65-2.77 (m, 2H), 2.39-2.46 (m, 2H) ppm. LC-MS (ESI+): 656.1 (M+H+).

Compound 228: (45.1 mg, 0.069 mmol, 18% yield, 99% purity, 99% ee, OR: −54.0). 1H NMR (DMSO-d₆, 400 MHz) δ 8.35 (br d, J=7.7 Hz, 1H), 7.98 (d, J=8.2 Hz, 2H), 7.85 (d, J=8.4 Hz, 2H), 7.68-7.77 (m, 2H), 7.41-7.53 (m, 4H), 7.29-7.35 (m, 2H), 7.17-7.28 (m, 3H), 5.05 (dd, J=11.1, 4.3 Hz, 1H), 4.42-4.54 (m, 2H), 3.98-4.12 (m, 1H), 3.90 (dd, J=11.2, 4.2 Hz, 1H), 2.65-2.78 (m, 2H), 2.40-2.45 (m, 2H) ppm. LC-MS (ESI+): 656.0 (M+H+).

### Synthesis of (1R,3s)-3-((S,E)-N′-((4-chlorophenyl)sulfonyl)-3-(4-fluorophenyl)-4-phenyl-4,5-dihydro-1H-pyrazole-1-carboximidamido)cyclobutyl sulfamate (Compound 229) & (1R,3s)-3-((R,E)-N′-((4-chlorophenyl)sulfonyl)-3-(4-fluorophenyl)-4-phenyl-4,5-dihydro-1H-pyrazole-1-carboximidamido)cyclobutyl sulfamate (Compound 230)

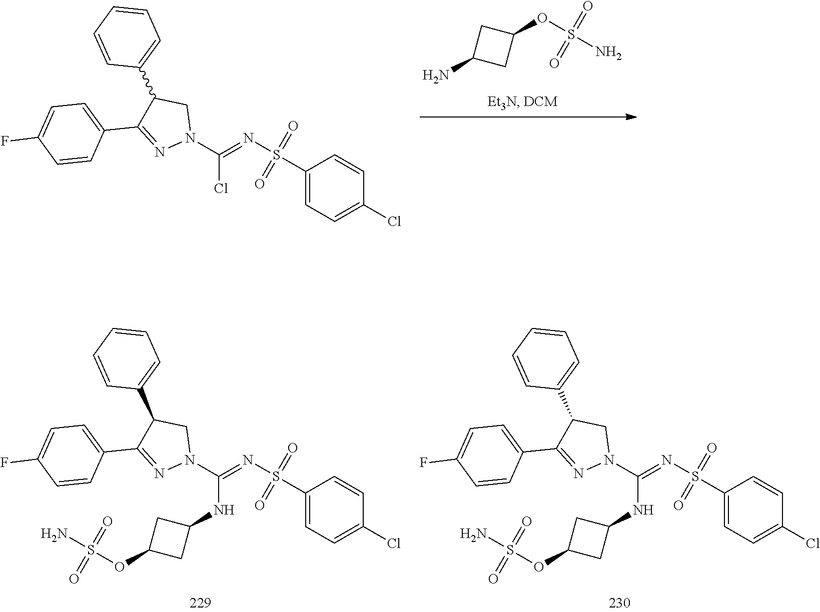 [SMILES: O=S(=O)(/N=C(\Cl)N1CC(c2ccccc2)C(c2ccc(F)cc2)=N1)c1ccc(Cl)cc1 ; NS(=O)(=O)O[C@H]1C[C@@H](N)C1 ; NS(=O)(=O)O[C@H]1C[C@@H](N/C(=N\S(=O)(=O)c2ccc(Cl)cc2)N2C[C@H](c3ccccc3)C(c3ccc(F)cc3)=N2)C1 ; NS(=O)(=O)O[C@H]1C[C@@H](N/C(=N\S(=O)(=O)c2ccc(Cl)cc2)N2C[C@@H](c3ccccc3)C(c3ccc(F)cc3)=N2)C1]

To a stirred solution of sulfamic acid 3-amino-cyclobutyl ester (140 mg, 0.84 mmol, 2 eq.) and triethylamine (0.17 mL, 1.3 mmol, 3.0 eq.) in dichloromethane (5 mL) was added (E)-3-(4-chlorophenyl)-N-((4-fluorophenyl)sulfonyl)-4-phenyl-4,5-dihydro-1H-pyrazole-1-carbimidoyl chloride (200 mg, 0.42 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 60/40, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 229: (42.4 mg, 0.07 mmol, 16% yield, 99% purity, 99% ee, OR: +120.8). 1H NMR (DMSO-d₆, 400 MHz) δ 8.24 (br d, J=7.2 Hz, 1H), 7.74-7.86 (m, 4H), 7.46-7.57 (m, 4H), 7.29-7.36 (m, 2H), 7.17-7.29 (m, 5H), 5.05 (dd, J=11.1, 4.1 Hz, 1H), 4.40-4.54 (m, 2H), 3.98-4.09 (m, 1H), 3.93 (dd, J=11.3, 4.3 Hz, 1H), 2.65-2.77 (m, 2H), 2.43 (br d, J=9.2 Hz, 2H) ppm. LC-MS (ESI+): 606.1 (M+H+).

Compound 230: (37.2 mg, 0.061 mmol, 15% yield, 99% purity, 99% ee, OR: −83.7). 1H NMR (DMSO-d₆, 400 MHz) δ 8.24 (br d, J=7.8 Hz, 1H), 7.75-7.87 (m, 4H), 7.45-7.61 (m, 4H), 7.29-7.36 (m, 2H), 7.17-7.28 (m, 5H), 5.05 (dd, J=11.2, 4.3 Hz, 1H), 4.41-4.55 (m, 2H), 3.98-4.09 (m, 1H), 3.93 (dd, J=11.3, 4.3 Hz, 1H), 2.65-2.78 (m, 2H), 2.38-2.45 (m, 2H) ppm. LC-MS (ESI+): 606.1 (M+H+).

### Synthesis of (S,E)-N′-((4-chlorophenyl)sulfonyl)-3-(4-fluorophenyl)-4-phenyl-N-((1r,3S)-3-(sulfamoylamino)cyclobutyl)-4,5-dihydro-1H-pyrazole-1-carboximidamide (Compound 231) & (R,E)-N′-((4-chlorophenyl)sulfonyl)-3-(4-fluorophenyl)-4-phenyl-N-((1r,3S)-3-(sulfamoylamino)cyclobutyl)-4,5-dihydro-1H-pyrazole-1-carboximidamide (Compound 232)

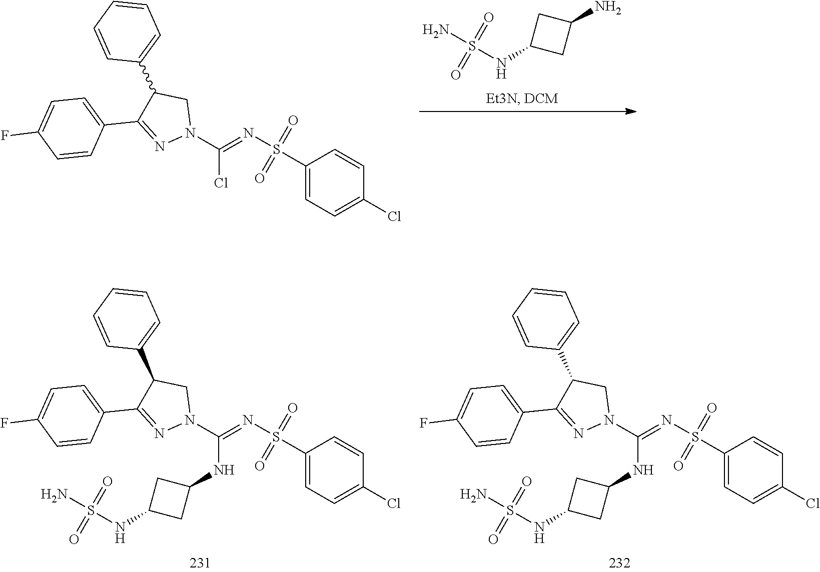 [SMILES: O=S(=O)(/N=C(\Cl)N1CC(c2ccccc2)C(c2ccc(F)cc2)=N1)c1ccc(Cl)cc1 ; NS(=O)(=O)N[C@H]1C[C@H](N)C1 ; NS(=O)(=O)N[C@H]1C[C@H](N/C(=N\S(=O)(=O)c2ccc(Cl)cc2)N2C[C@H](c3ccccc3)C(c3ccc(F)cc3)=N2)C1 ; NS(=O)(=O)N[C@H]1C[C@H](N/C(=N\S(=O)(=O)c2ccc(Cl)cc2)N2C[C@@H](c3ccccc3)C(c3ccc(F)cc3)=N2)C1]

To a stirred solution of ((1 r,3r)-3-aminocyclobutyl)sulfamide hydrochloride (117 mg, 0.58 mmol, 1.1 eq.) and triethylamine (0.26 mL, 1.8 mmol, 3.5 eq.) in dichloromethane (5 mL) was added (Z)-3-(4-chlorophenyl)-N-((4-fluorophenyl)sulfonyl)-4-phenyl-4,5-dihydro-1H-pyrazole-1-carbimidoyl chloride (250 mg, 0.53 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 231: (108.9 mg, 0.18 mmol, 34% yield, 95% purity, 99% ee, OR: −93.1). 1H NMR (DMSO-d₆, 400 MHz) δ 7.84-8.21 (m, 1H), 7.78 (br d, J=8.5 Hz, 4H), 7.53 (d, J=8.5 Hz, 2H), 7.29-7.36 (m, 2H), 7.17-7.28 (m, 5H), 6.77-7.04 (m, 1H), 6.31-6.70 (m, 2H), 4.92-5.08 (m, 1H), 4.35-4.51 (m, 2H), 3.79-3.99 (m, 2H), 2.27-2.44 (m, 4H) ppm. LC-MS (ESI+): 605.1 (M+H⁺).

Compound 232: (106.2 mg, 0.17 mmol, 33% yield, 95% purity, 99% ee, OR: +100.5). 1H NMR (DMSO-d₆, 400 MHz) δ 8.03 (br s, 1H), 7.78 (br d, J=8.6 Hz, 4H), 7.53 (d, J=8.5 Hz, 2H), 7.28-7.36 (m, 2H), 7.18-7.28 (m, 5H), 6.75-7.06 (m, 1H), 6.26-6.72 (m, 2H), 5.01 (br d, J=7.9 Hz, 1H), 4.33-4.53 (m, 2H), 3.77-4.00 (m, 2H), 2.27-2.41 (m, 4H) ppm. LC-MS (ESI+): 605.1 (M+H⁺).

### Synthesis of (S,E)-3-(4-chlorophenyl)-N′-((4-chlorophenyl)sulfonyl)-4-phenyl-N-((1s,3R)-3-(sulfamoylamino)cyclobutyl)-4,5-dihydro-1H-pyrazole-1-carboximidamide (Compound 233) & (R,E)-3-(4-chlorophenyl)-N′-((4-chlorophenyl)sulfonyl)-4-phenyl-N-((1s,3R)-3-(sulfamoylamino)cyclobutyl)-4,5-dihydro-1H-pyrazole-1-carboximidamide (Compound 234)

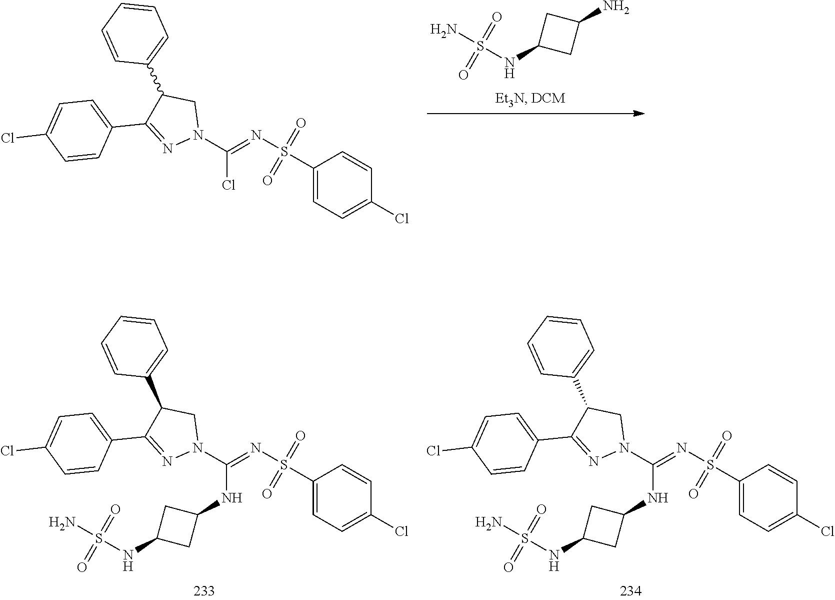 [SMILES: O=S(=O)(/N=C(\Cl)N1CC(c2ccccc2)C(c2ccc(Cl)cc2)=N1)c1ccc(Cl)cc1 ; NS(=O)(=O)N[C@H]1C[C@@H](N)C1 ; NS(=O)(=O)N[C@H]1C[C@@H](N/C(=N\S(=O)(=O)c2ccc(Cl)cc2)N2C[C@H](c3ccccc3)C(c3ccc(Cl)cc3)=N2)C1 ; NS(=O)(=O)N[C@H]1C[C@@H](N/C(=N\S(=O)(=O)c2ccc(Cl)cc2)N2C[C@@H](c3ccccc3)C(c3ccc(Cl)cc3)=N2)C1]

To a stirred solution of ((1s,3s)-3-aminocyclobutyl)sulfamide hydrochloride (113 mg, 0.56 mmol, 1.1 eq.) and triethylamine (0.25 mL, 1.77 mmol, 3.5 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 233: (73.1 mg, 0.12 mmol, 23% yield, 95% purity, 99% ee, OR: +72.4). 1H NMR (DMSO-d₆, 400 MHz) δ 7.97 (br s, 1H), 7.78 (d, J=8.6 Hz, 2H), 7.72 (br d, J=7.2 Hz, 2H), 7.54 (d, J=8.5 Hz, 2H), 7.44 (br d, J=8.6 Hz, 2H), 7.28-7.36 (m, 2H), 7.17-7.28 (m, 3H), 6.78 (br s, 1H), 6.53 (br s, 2H), 5.02 (br d, J=7.9 Hz, 1H), 4.45 (br t, J=11.3 Hz, 1H), 3.97-4.09 (m, 1H), 3.91 (br dd, J=10.9, 3.4 Hz, 1H), 3.34-3.43 (m, 1H), 2.56-2.69 (m, 2H), 1.97-2.16 (m, 2H) ppm. LC-MS (ESI+): 621.1 (M+H+).

Compound 234: (71.4 mg, 0.11 mmol, 22% yield, 95% purity, 99% ee, OR: −77.4). 1H NMR (DMSO-d₆, 400 MHz) δ 7.82-8.21 (m, 1H), 7.75-7.81 (m, 2H), 7.71 (br d, J=7.1 Hz, 2H), 7.54 (d, J=8.6 Hz, 2H), 7.44 (br d, J=8.5 Hz, 2H), 7.29-7.36 (m, 2H), 7.17-7.28 (m, 3H), 6.78 (br d, J=2.6 Hz, 1H), 6.54 (br s, 2H), 5.02 (br d, J=7.6 Hz, 1H), 4.45 (br t, J=11.3 Hz, 1H), 3.97-4.12 (m, 1H), 3.91 (br dd, J=11.1, 3.1 Hz, 1H), 3.35-3.43 (m, 1H), 2.55-2.69 (m, 2H), 1.98-2.14 (m, 2H) ppm. LC-MS (ESI+): 621.1 (M+H+).

### Synthesis of (S,E)-3-(4-chlorophenyl)-4-phenyl-N-((1-(sulfamoylamino)cyclopropyl)methyl)-N′-((4-(trifluoromethyl)phenyl)sulfonyl)-4,5-dihydro-1H-pyrazole-1-carboximidamide (Compound 235) & (R,E)-3-(4-chlorophenyl)-4-phenyl-N-((1-(sulfamoylamino)cyclopropyl)methyl)-N′-((4-(trifluoromethyl)phenyl)sulfonyl)-4,5-dihydro-1H-pyrazole-1-carboximidamide (Compound 236)

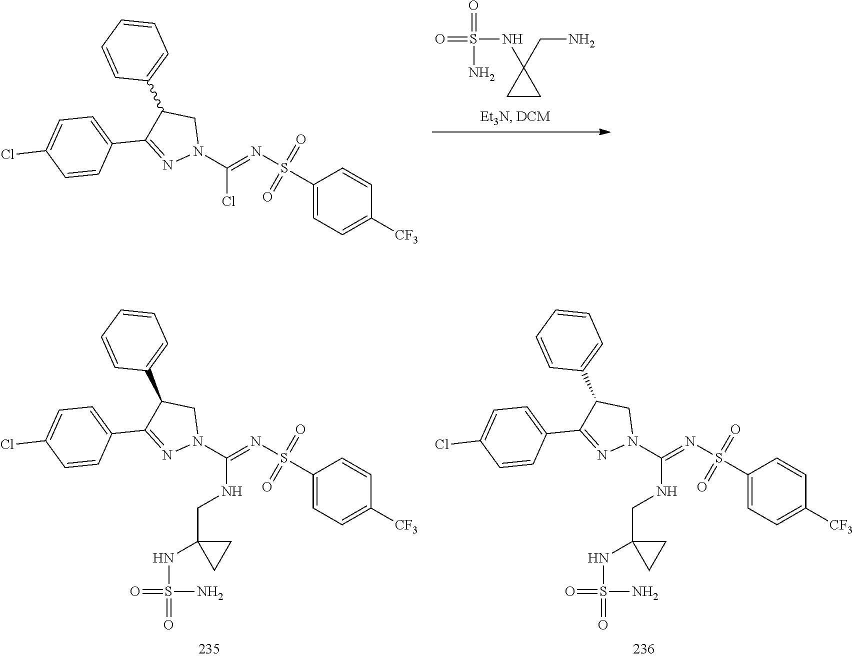 [SMILES: O=S(=O)(/N=C(\Cl)N1CC(c2ccccc2)C(c2ccc(Cl)cc2)=N1)c1ccc(C(F)(F)F)cc1 ; NCC1(NS(N)(=O)=O)CC1 ; NS(=O)(=O)NC1(CN/C(=N\S(=O)(=O)c2ccc(C(F)(F)F)cc2)N2C[C@H](c3ccccc3)C(c3ccc(Cl)cc3)=N2)CC1 ; NS(=O)(=O)NC1(CN/C(=N\S(=O)(=O)c2ccc(C(F)(F)F)cc2)N2C[C@@H](c3ccccc3)C(c3ccc(Cl)cc3)=N2)CC1]

To a stirred solution of (1-(aminomethyl)cyclopropyl)sulfamide (86 mg, 0.52 mmol, 1.1 eq.) and triethylamine (0.23 mL, 1.66 mmol, 3.5 eq.) in dichloromethane (5 mL) was added (Z)-3-(4-chlorophenyl)-4-phenyl-N-((4-(trifluoromethyl)phenyl)sulfonyl)-4,5-dihydro-1H-pyrazole-1-carbimidoyl chloride (250 mg, 0.47 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 235: (57.4 mg, 0.09 mmol, 18% yield, 95% purity, 99% ee, OR: −86.2). 1H NMR (DMSO-d₆, 400 MHz) δ 8.01 (d, J=8.1 Hz, 2H), 7.93 (br s, 1H), 7.85 (d, J=8.3 Hz, 2H), 7.67 (br d, J=8.4 Hz, 2H), 7.43 (d, J=8.6 Hz, 2H), 7.29-7.36 (m, 3H), 7.20-7.28 (m, 3H), 6.73 (br s, 2H), 5.09 (br dd, J=11.1, 4.2 Hz, 1H), 4.52 (t, J=11.1 Hz, 1H), 4.02 (br dd, J=11.0, 3.9 Hz, 1H), 3.55 (br s, 2H), 0.98 (s, 2H), 0.61 (br s, 2H) ppm. LC-MS (ESI+): 655.1 (M+H+).

Compound 236: (43.4 mg, 0.07 mmol, 14% yield, 95% purity, 99% ee, OR: +96.1). 1H NMR (DMSO-d₆, 400 MHz) δ 8.01 (d, J=8.1 Hz, 2H), 7.93 (br s, 1H), 7.85 (d, J=8.3 Hz, 2H), 7.67 (d, J=8.5 Hz, 2H), 7.43 (d, J=8.6 Hz, 2H), 7.29-7.36 (m, 3H), 7.21-7.28 (m, 3H), 6.73 (br s, 2H), 5.09 (br dd, J=11.1, 4.2 Hz, 1H), 4.52 (t, J=11.2 Hz, 1H), 4.02 (br dd, J=11.0, 4.0 Hz, 1H), 3.56 (br s, 2H), 0.98 (s, 2H), 0.61 (br s, 2H) ppm. LC-MS (ESI+): 655.1 (M+H+)

### Synthesis of (S,E)-3-(4-chlorophenyl)-N′-((4-chlorophenyl)sulfonyl)-4-phenyl-N-((1-(sulfamoylamino)cyclopropyl)methyl)-4,5-dihydro-1H-pyrazole-1-carboximidamide (Compound 237) & (R,E)-3-(4-chlorophenyl)-N′-((4-chlorophenyl)sulfonyl)-4-phenyl-N-((1-(sulfamoylamino)cyclopropyl)methyl)-4,5-dihydro-1H-pyrazole-1-carboximidamide (Compound 238)

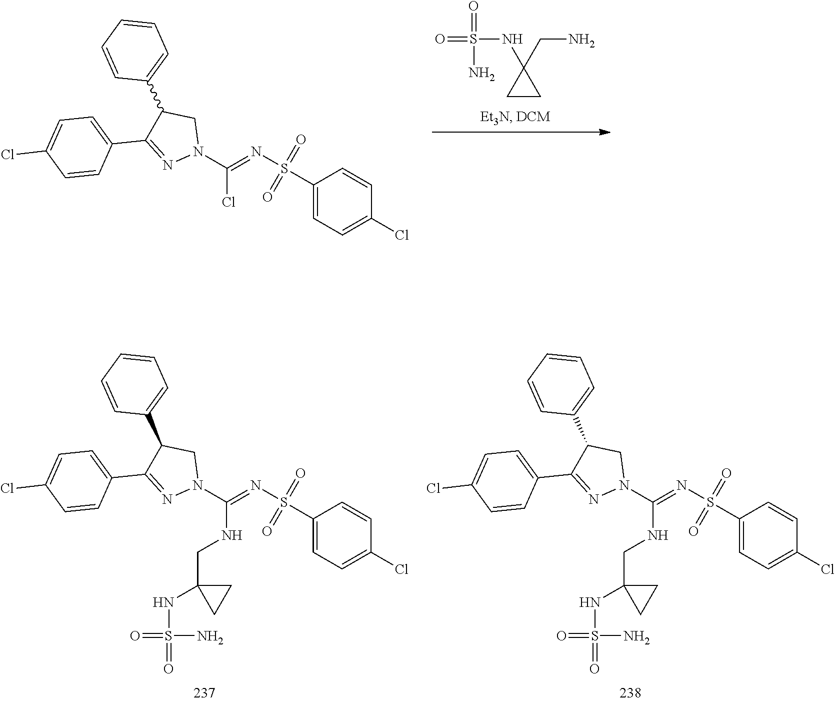 [SMILES: O=S(=O)(/N=C(\Cl)N1CC(c2ccccc2)C(c2ccc(Cl)cc2)=N1)c1ccc(Cl)cc1 ; NCC1(NS(N)(=O)=O)CC1 ; NS(=O)(=O)NC1(CN/C(=N\S(=O)(=O)c2ccc(Cl)cc2)N2C[C@H](c3ccccc3)C(c3ccc(Cl)cc3)=N2)CC1 ; NS(=O)(=O)NC1(CN/C(=N\S(=O)(=O)c2ccc(Cl)cc2)N2C[C@@H](c3ccccc3)C(c3ccc(Cl)cc3)=N2)CC1]

To a stirred solution of (1-(aminomethyl)cyclopropyl)sulfamide (92 mg, 0.56 mmol, 1.1 eq.) and triethylamine (0.25 mL, 1.77 mmol, 3.5 eq.) in dichloromethane (5 mL) was added (Z)-N-((4-chlorophenyl)sulfonyl)-3-(4-chlorophenyl)-4-phenyl-4,5-dihydro-1H-pyrazole-1-carbimidoyl chloride (250 mg, 0.51 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two enantiomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 237: (81.2 mg, 0.13 mmol, 26% yield, 95% purity, 99% ee, OR: −60.7). 1H NMR (DMSO-d₆, 400 MHz) δ 7.89 (br s, 1H), 7.80 (d, J=8.6 Hz, 2H), 7.70 (d, J=8.6 Hz, 2H), 7.51-7.56 (m, 2H), 7.44 (d, J=8.7 Hz, 2H), 7.29-7.37 (m, 3H), 7.19-7.29 (m, 3H), 6.73 (br s, 2H), 5.08 (br dd, J=11.2, 4.3 Hz, 1H), 4.51 (t, J=11.2 Hz, 1H), 4.03 (br dd, J=10.8, 3.9 Hz, 1H), 3.56 (br s, 2H), 0.99 (s, 2H), 0.62 (br t, J=4.6 Hz, 2H) ppm. LC-MS (ESI+): 621.1 (M+H+).

Compound 238: (90.1 mg, 0.14 mmol, 29% yield, 95% purity, 99% ee, OR: +90.6). 1H NMR (DMSO-d₆, 400 MHz) δ 7.89 (br s, 1H), 7.80 (d, J=8.6 Hz, 2H), 7.70 (br d, J=8.5 Hz, 2H), 7.54 (d, J=8.6 Hz, 2H), 7.44 (d, J=8.6 Hz, 2H), 7.29-7.37 (m, 3H), 7.20-7.28 (m, 3H), 6.73 (br s, 2H), 5.08 (br dd, J=11.1, 4.2 Hz, 1H), 4.51 (br t, J=11.2 Hz, 1H), 4.03 (br dd, J=10.7, 3.6 Hz, 1H), 3.56 (br s, 2H), 0.99 (br s, 2H), 0.62 (br s, 2H) ppm. LC-MS (ESI+): 621.1 (M+H+).

### Synthesis of (S,E)-3-(4-chlorophenyl)-N′-((4-chlorophenyl)sulfonyl)-4-phenyl-N-((1r,3S)-3-(sulfamoylamino)cyclobutyl)-4,5-dihydro-1H-pyrazole-1-carboximidamide (Compound 239) & (R,E)-3-(4-chlorophenyl)-N′-((4-chlorophenyl)sulfonyl)-4-phenyl-N-((1r,3S)-3-(sulfamoylamino)cyclobutyl)-4,5-dihydro-1H-pyrazole-1-carboximidamide (Compound 240)

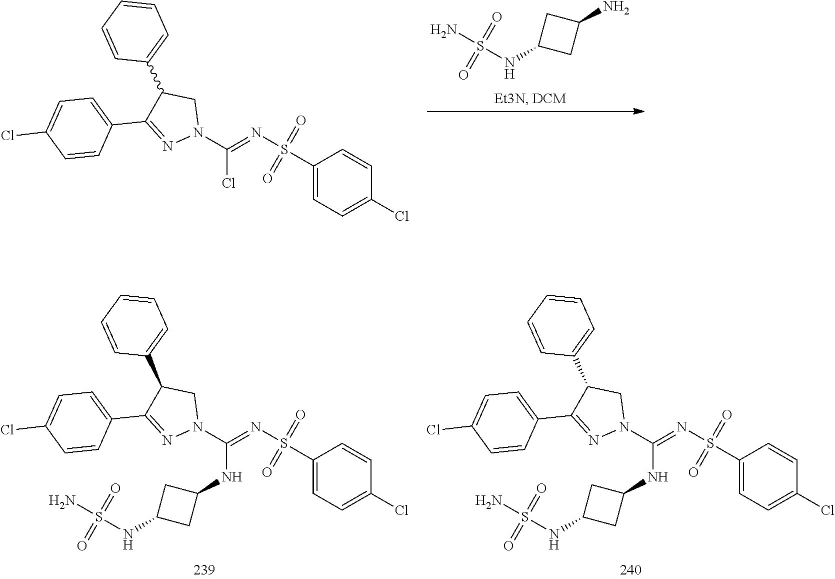 [SMILES: O=S(=O)(/N=C(\Cl)N1CC(c2ccccc2)C(c2ccc(Cl)cc2)=N1)c1ccc(Cl)cc1 ; NS(=O)(=O)N[C@H]1C[C@H](N)C1 ; NS(=O)(=O)N[C@H]1C[C@H](N/C(=N\S(=O)(=O)c2ccc(Cl)cc2)N2C[C@H](c3ccccc3)C(c3ccc(Cl)cc3)=N2)C1 ; NS(=O)(=O)N[C@H]1C[C@H](N/C(=N\S(=O)(=O)c2ccc(Cl)cc2)N2C[C@@H](c3ccccc3)C(c3ccc(Cl)cc3)=N2)C1]

To a stirred solution of ((1 r,3r)-3-aminocyclobutyl)sulfamide hydrochloride (105 mg, 0.52 mmol, 1.1 eq.) and triethylamine (0.23 mL, 1.7 mmol, 3.5 eq.) in dichloromethane (5 mL) was added (Z)-3-(4-chlorophenyl)-4-phenyl-N-((4-(trifluoromethyl)phenyl)sulfonyl)-4,5-dihydro-1H-pyrazole-1-carbimidoyl chloride (250 mg, 0.48 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 239: (85.1 mg, 0.13 mmol, 27% yield, 95% purity, 99% ee, OR: −86.3). 1H NMR (DMSO-d₆, 400 MHz) δ 8.15 (br s, 1H), 7.98 (d, J=8.1 Hz, 2H), 7.85 (d, J=8.3 Hz, 2H), 7.72 (d, J=8.6 Hz, 2H), 7.44 (d, J=8.7 Hz, 2H), 7.29-7.36 (m, 2H), 7.24-7.28 (m, 1H), 7.18-7.24 (m, 2H), 6.92 (d, J=7.2 Hz, 1H), 6.52 (s, 2H), 5.04 (br dd, J=11.1, 4.1 Hz, 1H), 4.36-4.55 (m, 2H), 3.80-3.99 (m, 2H), 2.30-2.45 (m, 4H) ppm. LC-MS (ESI+): 655.1 (M+H+).

Compound 240: (74.8 mg, 0.13 mmol, 24% yield, 95% purity, 99% ee, OR: +90.1). 1H NMR (DMSO-d₆, 400 MHz) δ 8.15 (br s, 1H), 7.98 (d, J=8.2 Hz, 2H), 7.85 (d, J=8.4 Hz, 2H), 7.72 (d, J=8.6 Hz, 2H), 7.44 (d, J=8.6 Hz, 2H), 7.29-7.36 (m, 2H), 7.16-7.28 (m, 3H), 6.92 (d, J=7.2 Hz, 1H), 6.52 (s, 2H), 5.04 (dd, J=11.2, 4.2 Hz, 1H), 4.35-4.54 (m, 2H), 3.80-3.98 (m, 2H), 2.23-2.45 (m, 4H) ppm. LC-MS (ESI+): 655.1 (M+H+).

### Synthesis of (S,E)-3-(4-chlorophenyl)-N′-((4-chlorophenyl)sulfonyl)-4-phenyl-N-((1r,3S)-3-(sulfamoylamino)cyclobutyl)-4,5-dihydro-1H-pyrazole-1-carboximidamide (Compound 241) & (R,E)-3-(4-chlorophenyl)-N′-((4-chlorophenyl)sulfonyl)-4-phenyl-N-((1r,3S)-3-(sulfamoylamino)cyclobutyl)-4,5-dihydro-1H-pyrazole-1-carboximidamide (Compound 242)

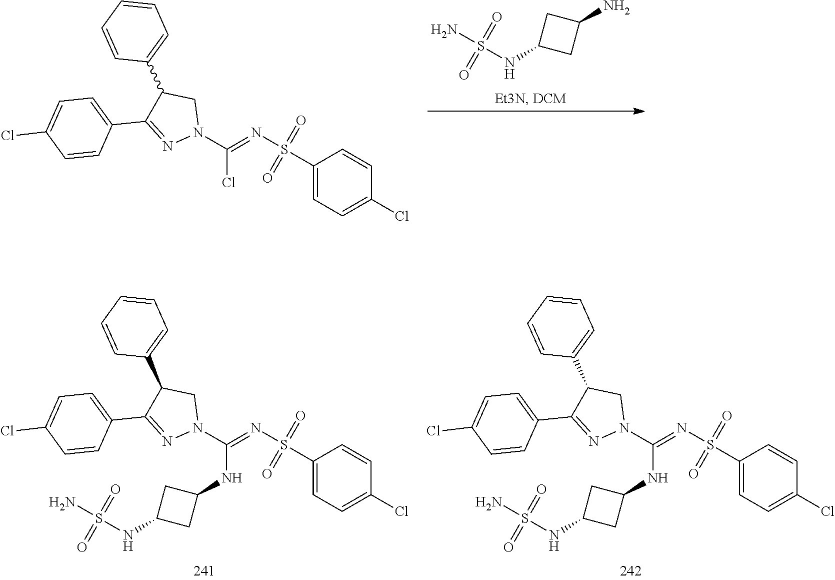 [SMILES: O=S(=O)(/N=C(\Cl)N1CC(c2ccccc2)C(c2ccc(Cl)cc2)=N1)c1ccc(Cl)cc1 ; NS(=O)(=O)N[C@H]1C[C@H](N)C1 ; NS(=O)(=O)N[C@H]1C[C@H](N/C(=N\S(=O)(=O)c2ccc(Cl)cc2)N2C[C@H](c3ccccc3)C(c3ccc(Cl)cc3)=N2)C1 ; NS(=O)(=O)N[C@H]1C[C@H](N/C(=N\S(=O)(=O)c2ccc(Cl)cc2)N2C[C@@H](c3ccccc3)C(c3ccc(Cl)cc3)=N2)C1]

To a stirred solution of ((1 r,3r)-3-aminocyclobutyl)sulfamide hydrochloride (113 mg, 0.56 mmol, 1.1 eq.) and triethylamine (0.25 mL, 1.8 mmol, 3.5 eq.) in dichloromethane (5 mL) was added (Z)-3-(4-chlorophenyl)-N-((4-chlorophenyl)sulfonyl)-4-phenyl-4,5-dihydro-1H-pyrazole-1-carbimidoyl chloride (250 mg, 0.51 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 241: (92.8 mg, 0.15 mmol, 29% yield, 95% purity, 99% ee, OR: −85.8). 1H NMR (DMSO-d₆, 400 MHz) δ 8.06 (br s, 1H), 7.76 (dd, J=17.6, 8.6 Hz, 4H), 7.54 (d, J=8.6 Hz, 2H), 7.45 (d, J=8.7 Hz, 2H), 7.30-7.36 (m, 2H), 7.26 (d, J=7.2 Hz, 1H), 7.18-7.23 (m, 2H), 6.93 (d, J=7.3 Hz, 1H), 6.52 (s, 2H), 5.03 (dd, J=11.1, 4.1 Hz, 1H), 4.32-4.56 (m, 2H), 3.79-4.06 (m, 2H), 2.24-2.46 (m, 4H) ppm. LC-MS (ESI+): 621.1 (M+H+).

Compound 242: (94.1 mg, 0.15 mmol, 30% yield, 95% purity, 99% ee, OR: +97.0). 1H NMR (DMSO-d₆, 400 MHz) δ 8.06 (br s, 1H), 7.76 (dd, J=17.5, 8.6 Hz, 4H), 7.54 (d, J=8.6 Hz, 2H), 7.45 (d, J=8.7 Hz, 2H), 7.29-7.36 (m, 2H), 7.26 (d, J=7.2 Hz, 1H), 7.17-7.23 (m, 2H), 6.93 (d, J=7.2 Hz, 1H), 6.52 (s, 2H), 5.03 (dd, J=11.2, 4.2 Hz, 1H), 4.46 (q, J=11.0 Hz, 2H), 3.78-4.03 (m, 2H), 2.28-2.45 (m, 4H) ppm. LC-MS (ESI+): 621.1 (M+H+).

### Synthesis of (S,E)-N′-((4-chlorophenyl)sulfonyl)-3-(4-fluorophenyl)-4-phenyl-N-((1s,3R)-3-(sulfamoylamino)cyclobutyl)-4,5-dihydro-1H-pyrazole-1-carboximidamide (Compound 243) & (R,E)-N′-((4-chlorophenyl)sulfonyl)-3-(4-fluorophenyl)-4-phenyl-N-((1s,3R)-3-(sulfamoylamino)cyclobutyl)-4,5-dihydro-1H-pyrazole-1-carboximidamide (Compound 244)

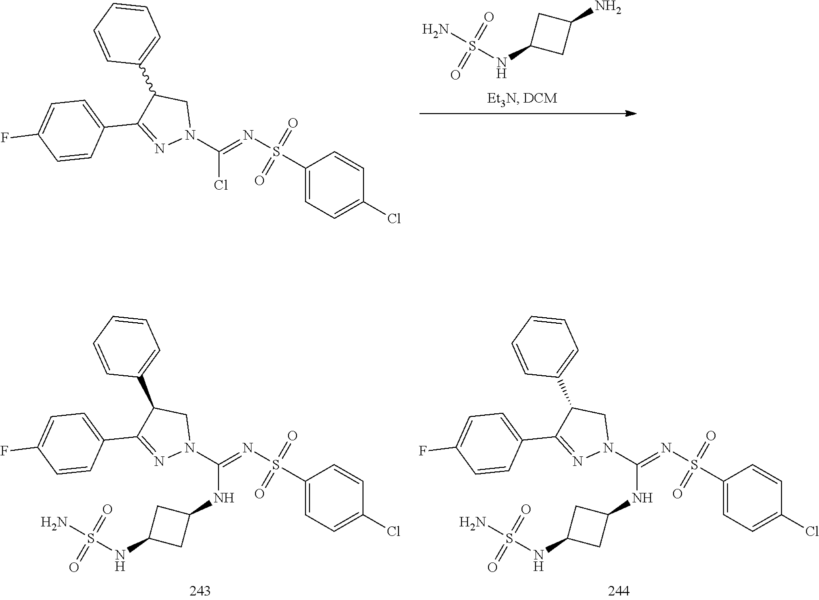 [SMILES: O=S(=O)(/N=C(\Cl)N1CC(c2ccccc2)C(c2ccc(F)cc2)=N1)c1ccc(Cl)cc1 ; NS(=O)(=O)N[C@H]1C[C@@H](N)C1 ; NS(=O)(=O)N[C@H]1C[C@@H](N/C(=N\S(=O)(=O)c2ccc(Cl)cc2)N2C[C@H](c3ccccc3)C(c3ccc(F)cc3)=N2)C1 ; NS(=O)(=O)N[C@H]1C[C@@H](N/C(=N\S(=O)(=O)c2ccc(Cl)cc2)N2C[C@@H](c3ccccc3)C(c3ccc(F)cc3)=N2)C1]

To a stirred solution of ((1s,3s)-3-aminocyclobutyl)sulfamide hydrochloride (117 mg, 0.58 mmol, 1.1 eq.) and triethylamine (0.26 mL, 1.84 mmol, 3.5 eq.) in dichloromethane (5 mL) was added (Z)-3-(4-chlorophenyl)-N-((4-fluorophenyl)sulfonyl)-4-phenyl-4,5-dihydro-1H-pyrazole-1-carbimidoyl chloride (250 mg, 0.53 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO₂ (40%)—ⁱPrOH+0.1% diethylamine, at 35° C., BPR 100 bar. Acquisition frequency was set to 220 nm for the DAD detector.

Compound 243: (62.1 mg, 0.1 mmol, 19% yield, 95% purity, 99% ee, OR: −130.0). 1H NMR (DMSO-d₆, 400 MHz) δ 7.93 (br s, 1H), 7.75-7.82 (m, 4H), 7.55 (d, J=8.6 Hz, 2H), 7.29-7.35 (m, 2H), 7.17-7.28 (m, 5H), 6.79 (d, J=8.0 Hz, 1H), 6.53 (s, 2H), 5.03 (dd, J=11.1, 4.2 Hz, 1H), 4.45 (t, J=11.3 Hz, 1H), 3.97-4.12 (m, 1H), 3.92 (br dd, J=11.2, 3.9 Hz, 1H), 3.35-3.45 (m, 1H), 2.56-2.71 (m, 2H), 1.97-2.16 (m, 2H) ppm. LC-MS (ESI+): 605.1 (M+H+).

Compound 244: (61.3 mg, 0.1 mmol, 19% yield, 95% purity, 99% ee, OR: +92.1). 1H NMR (DMSO-d₆, 400 MHz) δ 7.92 (br s, 1H), 7.73-7.81 (m, 4H), 7.54 (d, J=8.6 Hz, 2H), 7.28-7.36 (m, 2H), 7.17-7.28 (m, 5H), 6.79 (d, J=7.9 Hz, 1H), 6.53 (s, 2H), 5.03 (dd, J=11.1, 4.2 Hz, 1H), 4.45 (t, J=11.3 Hz, 1H), 3.97-4.12 (m, 1H), 3.91 (br dd, J=11.2, 4.1 Hz, 1H), 3.34-3.46 (m, 1H), 2.54-2.71 (m, 2H), 1.95-2.17 (m, 2H) ppm. LC-MS (ESI+): 605.1 (M+H+).

### Synthesis of (S,E)-3-(4-chlorophenyl)-4-phenyl-N-((1s,3R)-3-(sulfamoylamino)cyclobutyl)-N′-((4-(trifluoromethyl)phenyl)sulfonyl)-4,5-dihydro-1H-pyrazole-1-carboximidamide (Compound 245) & (R,E)-3-(4-chlorophenyl)-4-phenyl-N-((1s,3R)-3-(sulfamoylamino)cyclobutyl)-N′-((4-(trifluoromethyl)phenyl)sulfonyl)-4,5-dihydro-1H-pyrazole-1-carboximidamide (Compound 246)

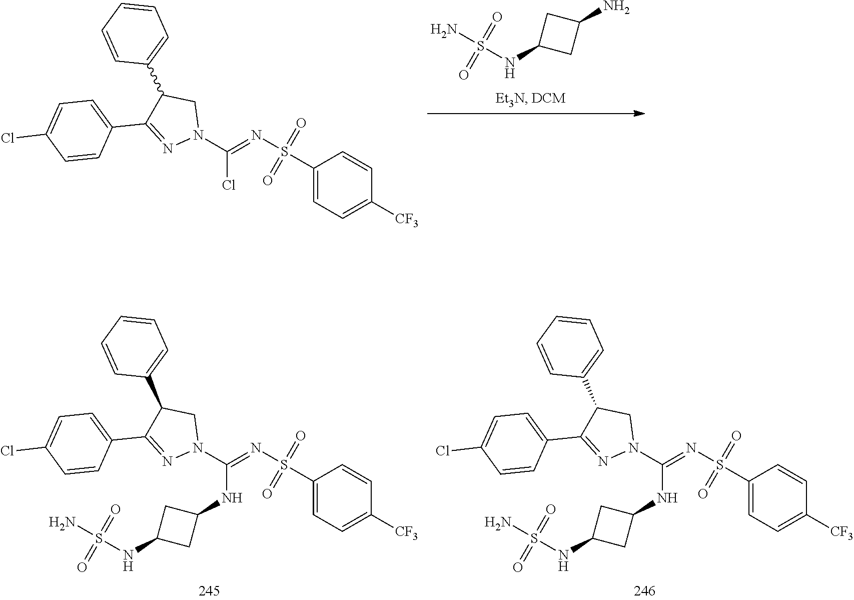 [SMILES: O=S(=O)(/N=C(\Cl)N1CC(c2ccccc2)C(c2ccc(Cl)cc2)=N1)c1ccc(C(F)(F)F)cc1 ; NS(=O)(=O)N[C@H]1C[C@@H](N)C1 ; NS(=O)(=O)N[C@H]1C[C@@H](N/C(=N\S(=O)(=O)c2ccc(C(F)(F)F)cc2)N2C[C@H](c3ccccc3)C(c3ccc(Cl)cc3)=N2)C1 ; NS(=O)(=O)N[C@H]1C[C@@H](N/C(=N\S(=O)(=O)c2ccc(C(F)(F)F)cc2)N2C[C@@H](c3ccccc3)C(c3ccc(Cl)cc3)=N2)C1]

To a stirred solution of ((1s,3s)-3-aminocyclobutyl)sulfamide hydrochloride (105 mg, 0.52 mmol, 1.1 eq.) and triethylamine (0.23 mL, 1.84 mmol, 3.5 eq.) in dichloromethane (5 mL) was added (Z)-3-(4-chlorophenyl)-4-phenyl-N-((4-(trifluoromethyl)phenyl)sulfonyl)-4,5-dihydro-1H-pyrazole-1-carbimidoyl chloride (250 mg, 0.47 mmol, 1.0 eq.). After stirring at rt for 18 h, the mixture was diluted with dichloromethane (20 mL) and washed with ammonium chloride (20 mL). The organic layer was separated, dried over magnesium sulfate, filtered, concentrated in vacuo, and purified by flash chromatography on silica eluting with ethyl acetate/heptane mixtures (0/100 to 70/30, v/v) to give a colorless oil, which was further subjected to SFC purification to resolve the two diastereomers as white solids.

SFC purification was performed on a Jasco 4000 Parallel SFC System (4× Channels 3×UV+1×DAD) using an Phenomenex column (Lux Amylose-1) 150 mm long×4.6 mm I.D. 5 μm particle size, on isocratic mode at 3 ml/min of CO2 (40%)—ⁱPrOH+0.1% diethylamine, at 35° C., BPR 100 bar. Acquisition frequency was set to 220 nm for the DAD detector.

Compound 245: (71.6 mg, 0.11 mmol, 23% yield, 97% purity, 99% ee, OR: −77.6). 1H NMR (DMSO-d₆, 400 MHz) δ 7.98 (d, J=8.1 Hz, 2H), 7.86 (d, J=8.3 Hz, 2H), 7.70 (d, J=8.5 Hz, 2H), 7.44 (d, J=8.6 Hz, 2H), 7.12-7.37 (m, 6H), 6.26-6.92 (m, 3H), 5.03 (dd, J=11.2, 4.2 Hz, 1H), 4.46 (t, J=11.3 Hz, 1H), 4.03 (quin, J=7.7 Hz, 1H), 3.90 (dd, J=11.2, 4.2 Hz, 1H), 3.36-3.43 (m, 1H), 2.55-2.69 (m, 2H), 2.01-2.12 (m, 2H) ppm. LC-MS (ESI+): 655.1 (M+H+).

Compound 246: (70.8 mg, 0.11 mmol, 23% yield, 97% purity, 99% ee, OR: +49.8). 1H NMR (DMSO-d₆, 400 MHz) δ 7.99 (d, J=8.1 Hz, 2H), 7.86 (d, J=8.3 Hz, 2H), 7.71 (br d, J=8.5 Hz, 2H), 7.44 (d, J=8.6 Hz, 2H), 7.15-7.41 (m, 6H), 6.37-6.93 (m, 3H), 5.03 (dd, J=11.2, 4.2 Hz, 1H), 4.47 (t, J=11.3 Hz, 1H), 4.04 (quin, J=7.6 Hz, 1H), 3.91 (dd, J=11.2, 4.2 Hz, 1H), 3.37-3.44 (m, 1H), 2.56-2.70 (m, 2H), 2.02-2.15 (m, 2H) ppm. LC-MS (ESI+): 655.1 (M+H+).

### Synthesis of (S,Z)-3-(4-chlorophenyl)-N′-((4-chlorophenyl)sulfonyl)-4-phenyl-N-((R)-1-sulfamoylpyrrolidin-3-yl)-4,5-dihydro-1H-pyrazole-1-carboximidamide (Compound 247) & (R,Z)-3-(4-chlorophenyl)-N′-((4-chlorophenyl)sulfonyl)-4-phenyl-N-((R)-1-sulfamoylpyrrolidin-3-yl)-4,5-dihydro-1H-pyrazole-1-carboximidamide (Compound 248)

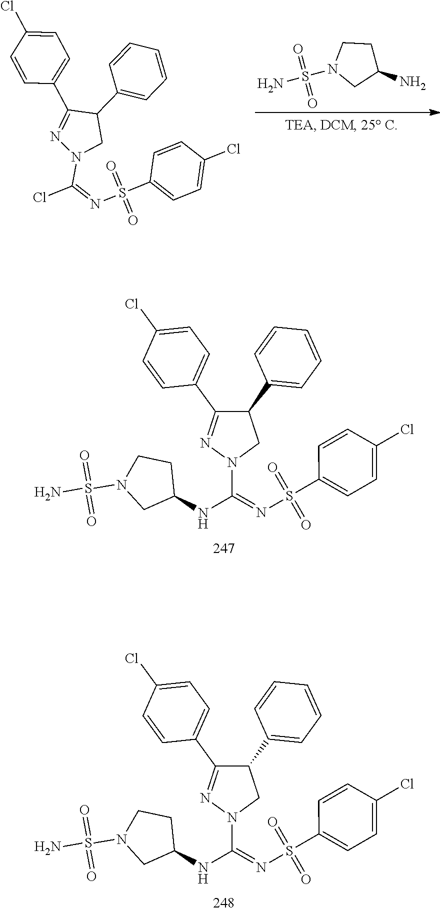 [SMILES: O=S(=O)(/N=C(/Cl)N1CC(c2ccccc2)C(c2ccc(Cl)cc2)=N1)c1ccc(Cl)cc1 ; N[C@@H]1CCN(S(N)(=O)=O)C1 ; NS(=O)(=O)N1CC[C@@H](N/C(=N/S(=O)(=O)c2ccc(Cl)cc2)N2C[C@H](c3ccccc3)C(c3ccc(Cl)cc3)=N2)C1 ; NS(=O)(=O)N1CC[C@@H](N/C(=N/S(=O)(=O)c2ccc(Cl)cc2)N2C[C@@H](c3ccccc3)C(c3ccc(Cl)cc3)=N2)C1]

To a solution of 1.0 (1.1 g, 2.23 mmol, 1.5 eq) in dichloromethane (10 mL) at rt were added triethylamine (752.62 mg, 7.44 mmol, 1.04 mL, 5 eq) and (R)-3-aminopyrrolidine-1-sulfonamide (300 mg, 1.49 mmol, 1 eq). After stirring at rt for 12 h, the mixture was concentrated under reduced pressure. The residue was purified by column on silica gel using petroleum ether and ethyl acetate (1/1, v/v) to give diastereomers (836 mg, 1.34 mmol, 80%) as a white solid. CMS (ESI+): m/z 621.0 (M+H)⁺.

The diastereomers (200 mg, 321.80 μmol, 1 eq) were separated by SFC to give Compound 247 (100 mg, 160.89 μmol, 99.8%) as a white solid and Compound 248 (100 mg, 146.41 μmol, 90.9%) as a white solid.

Method of the SFC: Column: Chiralpak AD-3, 50×4.6 mm I.D., 3 μm; Mobile phase A: CO2; Mobile phase B: ethanol (0.1% isopropanol m, v/v); B %: 50%, Run time: 15 min.

Compound 247: ¹H NMR: 400 MHz, DMSO-d₆ δ ppm 1.89-2.03 (m, 1H), 2.06-2.20 (m, 1H), 3.06-3.19 (m, 2H), 3.23-3.29 (m, 1H), 3.41 (dd, J=10.01, 7.00 Hz, 1H), 3.96 (dd, J=11.26, 4.25 Hz, 1H), 4.42-4.60 (m, 2H), 5.07 (dd, J=11.26, 4.25 Hz, 1H), 6.87 (s, 2H), 7.18-7.29 (m, 3H), 7.29-7.36 (m, 1H), 7.29-7.37 (m, 1H), 7.44 (d, J=9.01 Hz, 2H), 7.52-7.58 (m, 2H), 7.73 (d, J=8.50 Hz, 2H), 7.78-7.84 (m, 2H), 7.95 (br s, 1H); LCMS (ESI+): m/z 621.0 (M+H)⁺OR: −95.2.

Compound 248: ¹H NMR: 400 MHz, DMSO-d₆ δ ppm 1.96-2.06 (m, 1H), 2.07-2.21 (m, 1H), 3.04-3.17 (m, 2H), 3.24-3.30 (m, 1H), 3.35-3.41 (m, 1H), 4.03 (br dd, J=11.26, 3.75 Hz, 1H), 4.50 (br t, J=11.26 Hz, 2H), 5.07 (br dd, J=11.01, 4.00 Hz, 1H), 6.86 (br s, 2H), 7.20-7.28 (m, 3H), 7.30-7.36 (m, 2H), 7.44 (d, J=8.50 Hz, 2H), 7.55 (d, J=8.50 Hz, 2H), 7.73 (br d, J=8.50 Hz, 2H), 7.81 (d, J=8.50 Hz, 2H), 7.97 (br s, 1H); LCMS (ESI+): m/z 621.0 (M+H)⁺; OR: 89.6

### Synthesis of (S,Z)-3-(4-chlorophenyl)-N′-((4-chlorophenyl)sulfonyl)-4-phenyl-N-(1-sulfamoylpiperidin-4-yl)-4,5-dihydro-1H-pyrazole-1-carboximidamide (Compound 249) & (R,Z)-3-(4-chlorophenyl)-N′-((4-chlorophenyl)sulfonyl)-4-phenyl-N-(1-sulfamoylpiperidin-4-yl)-4,5-dihydro-1H-pyrazole-1-carboximidamide (Compound 250)

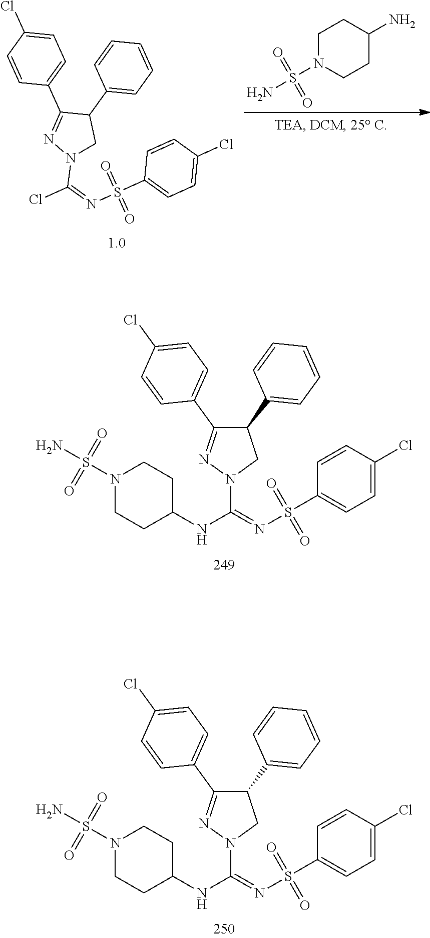 [SMILES: O=S(=O)(/N=C(/Cl)N1CC(c2ccccc2)C(c2ccc(Cl)cc2)=N1)c1ccc(Cl)cc1 ; NC1CCN(S(N)(=O)=O)CC1 ; NS(=O)(=O)N1CCC(N/C(=N/S(=O)(=O)c2ccc(Cl)cc2)N2C[C@H](c3ccccc3)C(c3ccc(Cl)cc3)=N2)CC1 ; NS(=O)(=O)N1CCC(N/C(=N/S(=O)(=O)c2ccc(Cl)cc2)N2C[C@@H](c3ccccc3)C(c3ccc(Cl)cc3)=N2)CC1]

To a solution of 1.0 (891.02 mg, 1.81 mmol, 1.3 eq) in dichloromethane (5 mL) at rt were added triethylamine (967.93 μL, 6.95 mmol, 5 eq) and 4-aminopiperidine-1-sulfonamide (300 mg, 1.39 mmol, 1 eq, HCl). After stirring at rt for 12 h, the mixture was concentrated under reduced pressure. The residue was purified by column on silica gel using petroleum ether and ethyl acetate (1/2, v/v) to give a crude, which was further separated by SFC to give Compound 250 (109 mg, 171.50 mmol, 12.34%) as a white solid and Compound 249 (135 mg, 212.40 mmol, 15.28%) as a white solid.

Compound 249: ¹H NMR: 400 MHz, DMSO-d₆ δ ppm 8.01-7.86 (m, 1H), 7.81-7.69 (m, 4H), 7.60-7.51 (m, 2H), 7.45 (d, J=8.7 Hz, 2H), 7.37-7.29 (m, 2H), 7.28-7.18 (m, 3H), 6.76 (s, 2H), 5.06 (dd, J=4.5, 11.1 Hz, 1H), 4.51 (t, J=11.2 Hz, 1H), 3.99 (br dd, J=4.3, 11.2 Hz, 1H), 3.87-3.69 (m, 1H), 3.45 (br d, J=9.8 Hz, 2H), 2.48-2.39 (m, 2H), 1.92-1.81 (m, 2H), 1.70 (dquin, J=3.7, 12.1 Hz, 2H) LCMS (ESI+): m/z 635.0 (M+H)⁺; OR: −81.20.

Compound 250: ¹H NMR: 400 MHz, DMSO-d₆ δ ppm 7.99-7.86 (m, 1H), 7.79 (d, J=8.6 Hz, 2H), 7.73 (br d, J=8.4 Hz, 2H), 7.54 (d, J=8.6 Hz, 2H), 7.44 (d, J=8.7 Hz, 2H), 7.36-7.30 (m, 2H), 7.27-7.18 (m, 3H), 6.75 (br s, 2H), 5.05 (br dd, J=4.2, 11.1 Hz, 1H), 4.50 (br t, J=11.2 Hz, 1H), 3.98 (br dd, J=3.5, 10.6 Hz, 1H), 3.79 (br s, 1H), 3.45 (br d, J=9.4 Hz, 2H), 2.47-2.39 (m, 2H), 1.93-1.81 (m, 2H), 1.75-1.62 (m, 2H); LCMS (ESI+): m/z 635.0 (M+H)⁺; OR: 78.60.

### Example 3. Biochemical Methods Human CB1 Receptor-1 (hCB1) and Human CB1 Receptor-1b (hCBib)

hCB1/hCB1 b Gai Cyclic AMP (cAMP) Assays: Cells were seeded in a 384-well plate at an appropriate density from frozen cells. The cAMP assays were performed according to the manufacturer's protocol using MULTISCREEN™ TR-FRET cAMP 1.0 No Wash Assay Kit. Cells (hCB1 or hCB1 b) were pre-incubated with customer compounds for 30 minutes at rt followed by addition of EC80 CP55940 concentration and Forskolin. Cells were then incubated for 20 minutes at 37C. The reaction was terminated by sequentially adding sequentially adding MULTISCREEN™ Eu-labeled cAMP and MULTISCREEN™ 650-labeled anti-cAMP antibody in lysis buffer. The plate was then incubated at rt for 30 minutes before reading fluorescent emissions at 620 nm and 665 with excitation at 314 nm on FlexStation III (Molecular Devices). The results are shown in Table 2 (“***” denotes an IC₅₀<100 nM; “**” denotes an IC₅₀ between 100 nM and 500 nM, and “*” denotes an IC₅₀>500 nM).

TABLE 2

| Example | CB1 IC₅₀ (nM) | CB1b IC₅₀ (nM) |
|---|---|---|
| 1 | *** | *** |
| 3 | * | ** |
| 5 | *** | ** |
| 6 | ** | *** |
| 9 | *** | *** |
| 11 | *** | *** |
| 13 | *** | *** |
| 14 | *** | *** |
| 17 | *** | * |
| 18 | *** | ** |
| 21 | *** | *** |
| 23 | ** | ** |
| 26 | *** | ** |
| 27 | *** | *** |
| 29 | *** | *** |
| 31 | *** | *** |
| 33 | ** | *** |
| 35 | *** | *** |
| 43 | *** | *** |
| 45 | *** | *** |
| 47 | *** | *** |
| 48 | *** | *** |
| 51 | ** | ** |
| 53 | *** | *** |
| 54 | *** | *** |
| 57 | * | * |
| 59 | ** | *** |
| 61 | ** | ** |
| 65 | *** | *** |
| 67 | * | * |
| 69 | * | * |
| 73 | ** | ** |
| 87 | ** | ** |
| 99 | * | * |
| 109 | * | * |
| 111 | * | * |
| 113 | * | * |
| 115 | *** | *** |
| 116 | ** | ** |
| 119 | * | * |
| 122 | ** | ** |
| 123 | * | * |
| 125 | ** | ** |
| 127 | ** | ** |
| 129 | * | * |
| 135 | * | * |
| 136 | * | * |
| 139 | ** | ** |
| 141 | ** | ** |
| 143 | *** | *** |
| 145 | ** | ** |
| 147 | ** | ** |
| 149 | ** | ** |
| 151 | ** | *** |
| 153 | ** | *** |
| 155 | *** | *** |
| 157 | *** | ** |
| 159 | *** | *** |
| 161 | ** | ** |
| 163 | *** | ** |
| 165 | *** | ** |
| 167 | *** | *** |
| 169 | *** | *** |
| 173 | ** | ** |
| 183 | *** | *** |
| 185 | ** | *** |
| 187 | ** | ** |
| 191 | *** | *** |
| 226 | * | *** |
| 227 | ** | |
| 228 | * | *** |
| 235 | ** | *** |
| 239 | ** | *** |
| 241 | ** | *** |
| 245 | ** | *** |
| 247 | ** | *** |

### Example 4. Efficacy study of experimental compound in combination with Semaglutide or Tirzepatide in C57BL/6J DIO mice

A study was performed to determine the efficacy of Compound 9 in inducing weight loss in mice alone, in combination with semaglutide, and in combination with tirzepatide.

### Study Procedure:

### DIO Mouse Generation

5-6 weeks age male C57BL/6J mice were fed a high fat pelleted diet D12492i (Research Diets, Inc) and fresh water, ad libitum in Gemapharmatech. The animal room environment was controlled (target conditions: temperature 20 to 24° C., relative humidity 30 to 70%). Temperature and relative humidity were monitored daily. An electronic time-controlled lighting system is used to provide a 12 hour light/12 hour dark cycle. 4 mice were housed per plastic cage which is in accordance with the National Research Council “Guide for the Care and Use of Laboratory Animals.” Enrichment toys were provided. Typically, 12 weeks later, C57BL/6J male mice become obese, mildly to moderately hyperglycemic and develop impaired glucose tolerance.

### 1. Acclimation

The mice were orally dosed with 40 mg/kg HPMC E3+0.5% HPMC BID, dose of volume 5 mL/kg and subdermal dosed with 20 mM citrate buffer at 5 mL/kg on a Q3D schedule for 1˜2 weeks to ensure a smooth dosing operation and to acclimate the mice to repeat dosing. Mice with body weights that have not stabilized by the end of the dosing acclimation were removed from the study.

### 2. Grouping

Day 0: All animals were assigned into 20 groups based on the body weight and random blood glucose, then the study was divided into two parts with 80 mice (n=4/gp*20 groups) in each part.

### 3. Dose

Day 1-28: Body weight were recorded daily before dosing in the morning.

BID dosing (2.5 mg/kg): Dosing time were 9:30 am-11:00 am and 5:30 pm-7:00 pm.

Q3D dosing: The dose time of Semaglutide and Tirzepatide were 5:30 pm dosed on day 1, 4, 7, 10,13,16,19,22,25, and 28.

4. Food intake

Day 1-28: The food intake of animal was measured daily before dosing in the morning. Animals were housed singly to allow for food intake measurements.

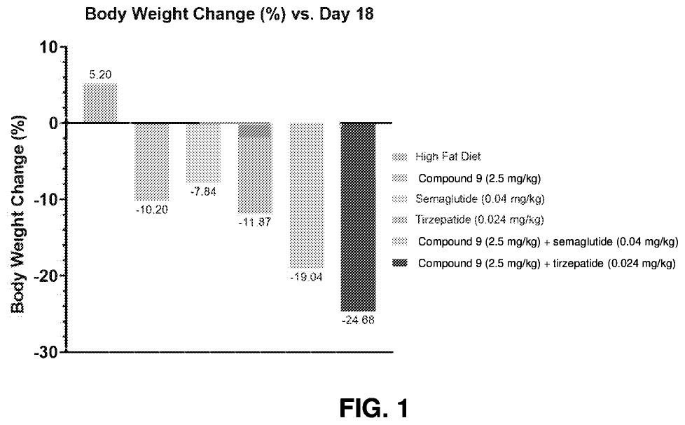

The results of the study are shown in FIG. 1.

### Example 5. Pharmacokinetics of Following Oral Administration in Male C57BL/6 Mice

A study was conducted to compare the brain/plasma ratios of Compound 9 and rimonabant following oral administration in mice.

### Study Details

Food Status

Fasted overnight with free access to water and fed 4 hr post dosing

Treatment Group

PO administration via oral gavage

Volume

Group 2: PO, 10 mg/kg, 10 mL/kg

Formulation

0.5% MC (400 cp) in purified water (1 mg/mL)

Sampling Design

Terminal sampling for brain @0.25, 0.5, 1, 2, 4, 8 and 24 hr, 7 time points. (N=3/timepoint, N=21/arm); collect brains after perfusion.

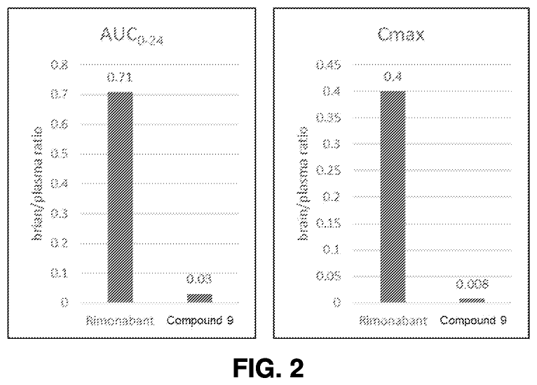

The results of the study are shown in FIG. 2. It was found that Compound 9 exhibited substantially reduced blood-brain-barrier penetration as compared to rimonabant, as evidenced by the AUC₀₋₂₄ and Cₘₐₓ values following single PO administration of each agent.

### OTHER EMBODIMENTS

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A compound of formula (I): 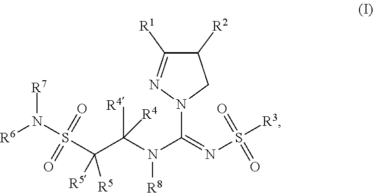
or a pharmaceutically acceptable salt thereof, wherein
R¹ is phenyl optionally substituted with one or more substituents selected from F, Cl, CN, and OCH₃;
R² is C₁-C₆ alkyl, 5- or 6-membered heteroaryl or phenyl optionally substituted with F or CN;
R³ is phenyl substituted with one or two substituents selected from F, Cl, CF₃, CN, OCH₃,C₂-C₆ alkynyl, and C(O)NH₂; or 5- or 6-membered heteroaryl containing 1-3 nitrogen atoms, wherein the heteroaryl is optionally substituted with C₁-C₆ alkyl;
R⁴, R^{4′}, R⁵, and R^{5′} are independently H or C₁-C₆ alkyl;
R⁶ and R⁷ are independently H, OH, or C₁-C₆ alkyl; or
R⁶ and R⁷, together with the nitrogen atom to which they are attached, form 5- or 6-membered heterocycloalkyl containing 1-2 nitrogen atoms and optionally substituted with C₁-C₆ alkyl; and
R⁸ is H or CH₃,
provided that at least one of the following is true:
(i) R¹ is not 4-chlorophenyl or 4-methoxyphenyl;
(ii) R² is C₁-C₆ alkyl or phenyl substituted with CN;
(iii) R³ is not 4-chlorophenyl, 3-cyanophenyl, or 3-methoxyphenyl;
(iv) at least one of R⁴, R^{4′}, R⁵, and R^{5′} is C₁-C₆ alkyl;
(v) at least one of R⁶ and R⁷ is not H; and
(vi) R⁸ is CH₃.

2. The compound of claim 1, wherein the compound is a compound of formula (IA): 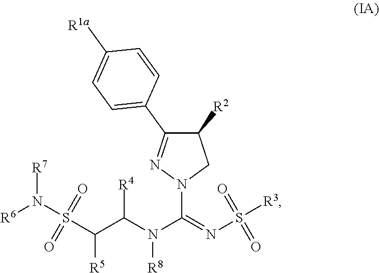
or a pharmaceutically acceptable salt thereof, wherein R^{1a} is F, Cl, CN, or OCH₃.

3. The compound of claim 2, wherein the compound is a compound of formula (IIA): 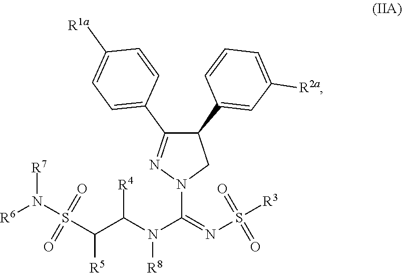
or a pharmaceutically acceptable salt thereof, wherein R^{2a} is H or CN.

4. The compound of claim 3, wherein R^{2a} is H.

5. The compound of claim 3, wherein R^{2a} is CN.

6. The compound of claim 2, wherein the compound is a compound of formula (IIIA): 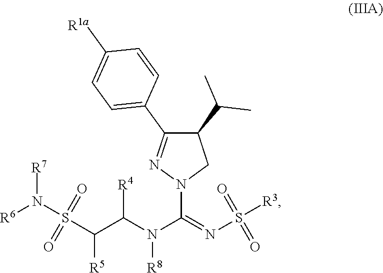
or a pharmaceutically acceptable salt thereof.

7. The compound of claim 1, wherein the compound is a compound of formula (IB): 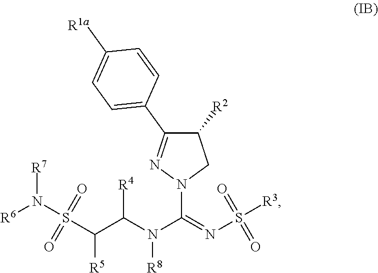
or a pharmaceutically acceptable salt thereof, wherein R^{1a} is F, Cl, CN, or OCH₃.

8. The compound of claim 7, wherein the compound is a compound of formula (IIB): 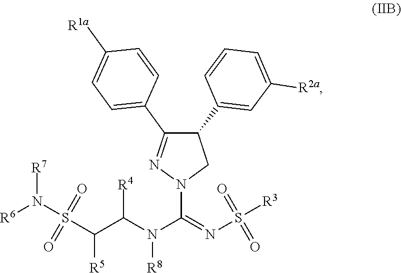
or a pharmaceutically acceptable salt thereof, wherein R^{2a} is H or CN.

9. The compound of claim 8, wherein R^{2a} is H.

10. The compound of claim 8, wherein R^{2a} is CN.

11. The compound of claim 7, wherein the compound is a compound of formula (IIIB): 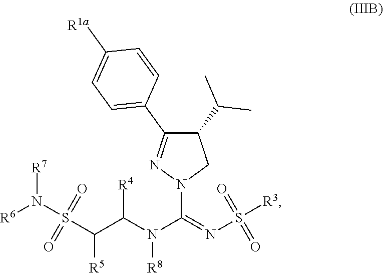
or a pharmaceutically acceptable salt thereof.

12. The compound of any one of claims 2-11, wherein R^{1a} is F.

13. The compound of any one of claims 2-11, wherein R^{1a} is Cl.

14. The compound of any one of claims 2-11, wherein R^{1a} is OCH₃.

15. The compound of any one of claims 1-14, wherein R³ is phenyl substituted with one or two groups selected from F, Cl, CF₃, CN, OCH₃, C₂-C₆ alkynyl, or C(O)NH₂.

16. The compound of claim 15, wherein R³ is:

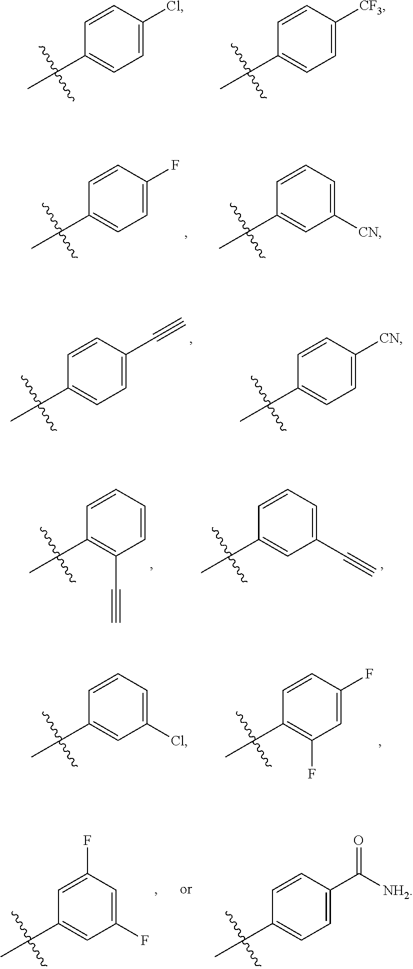

17. The compound of claim 16, wherein R³ is

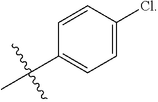 [SMILES: *c1ccc(Cl)cc1]

18. The compound of claim 16, wherein R³ is

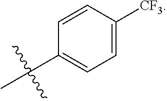 [SMILES: *c1ccc(C(F)(F)F)cc1]

19. The compound of claim 16, wherein R³ is

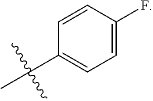 [SMILES: *c1ccc(F)cc1]

20. The compound of any one of claims 1-14, wherein R³ is 5- or 6-membered heteroaryl containing 1-3 nitrogen atoms, wherein the heteroaryl is optionally substituted with C₁-C₆ alkyl.

21. The compound of claim 20, wherein R³ is:

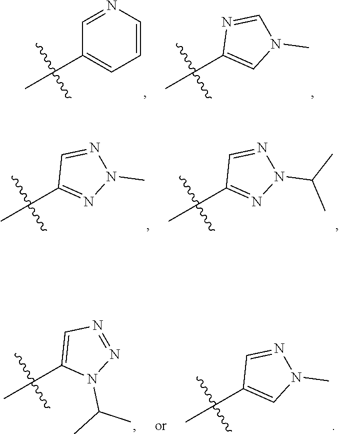

22. The compound of any one of claims 1-21, wherein R⁴ is H.

23. The compound of any one of claims 1-21, wherein R⁴ is C₁-C₆ alkyl.

24. The compound of claim 23, wherein R⁴ is methyl.

25. The compound of claim 23, wherein R⁴ is isopropyl.

26. The compound of any one of claims 1-25, wherein R⁵ is H.

27. The compound of any one of claims 1-25, wherein R⁵ is C₁-C₆ alkyl.

28. The compound of claim 27, wherein R⁵ is methyl.

29. The compound of claim 27, wherein R⁵ is isopropyl.

30. The compound of any one of claims 1-29, wherein R⁶ is H.

31. The compound of any one of claims 1-29, wherein R⁶ is C₁-C₆ alkyl.

32. The compound of claim 31, wherein R⁶ is methyl, ethyl, or isopropyl.

33. The compound of any one of claims 1-29, wherein R⁶ is OH.

34. The compound of any one of claims 1-33, wherein R⁷ is H.

35. The compound of any one of claims 1-33, wherein R⁷ is C₁-C₆ alkyl.

36. The compound of claim 35, wherein R⁷ is methyl, ethyl, or isopropyl.

37. The compound of any one of claims 1-33, wherein R⁷ is OH.

38. The compound of any one of claims 1-29, wherein R⁶ and R⁷, together with the nitrogen atom to which they are attached, form 5- or 6-membered heterocycloalkyl containing 1-2 nitrogen atoms and optionally substituted with C₁-C₆ alkyl.

39. The compound of claim 38, wherein R⁶ and R⁷, together with the nitrogen atom to which they are attached, form:

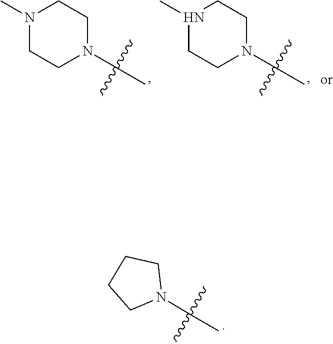

40. The compound of any one of claims 1-39, wherein R⁸ is H.

41. The compound of any one of claims 1-39, wherein R⁸ is CH₃.

42. The compound of claim 1, wherein the compound is selected from any of compounds 1-86, 103-110, 155-172, 175-182, or a pharmaceutically acceptable salt thereof.

43. A compound of formula (II): 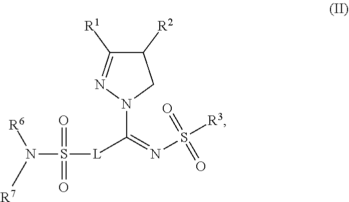
or a pharmaceutically acceptable salt thereof, wherein
R¹ is phenyl optionally substituted with one or more substituents selected from halogen, C₁-C₆ alkoxy, and CN;
R² is C₁-C₆ alkyl, 5- or 6-membered heteroaryl, or phenyl optionally substituted with halogen or CN;
R³ is phenyl substituted with one or more substituents selected from halogen, C₁-C₆ alkoxy, CF₃, CN, C₂-C₆ alkynyl, and C(O)NH₂; or 5- or 6-membered heteroaryl containing 1-3 nitrogen atoms, wherein the heteroaryl is optionally substituted with C₁-C₆ alkyl;
R⁶ and R⁷ are independently H, OH, or C₁-C₆ alkyl; or
R⁶ and R⁷, together with the nitrogen atom to which they are attached, form 5- or 6-membered heterocycloalkyl containing 1-2 nitrogen atoms and optionally substituted with C₁-C₆ alkyl;
L is 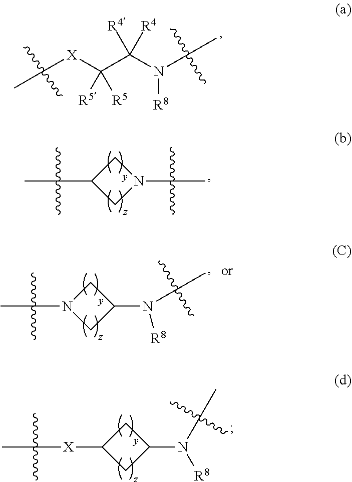
R⁸ is H or C₁-C₆ alkyl;
R⁴, R^{4′}, R⁵, and R^{5′} are independently H or C₁-C₆ alkyl optionally substituted with C₁-C₆ alkoxy;
X is O or NR⁹;
R⁹ is H or C₁-C₆ alkyl; and
y and z are independently 1, 2, or 3.

44. The compound of claim 43, wherein the compound is a compound of formula (IVA): 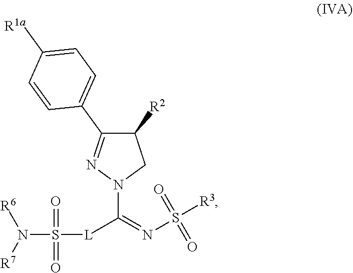
or a pharmaceutically acceptable salt thereof, wherein R^{1a} is halogen, CN, or C₁-C₆ alkoxy.

45. The compound of claim 44, wherein the compound is a compound of formula (VA): 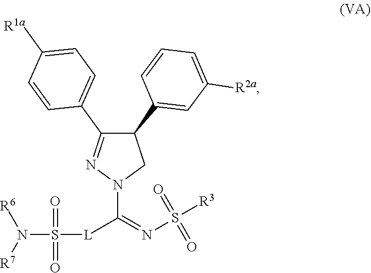
or a pharmaceutically acceptable salt thereof, wherein R^{2a} is H, halogen, C₁-C₆ alkoxy, or CN.

46. The compound of claim 45, wherein R^{2a} is H.

47. The compound of claim 45, wherein R^{2a} is CN.

48. The compound of claim 44, wherein the compound is a compound of formula (VIA): 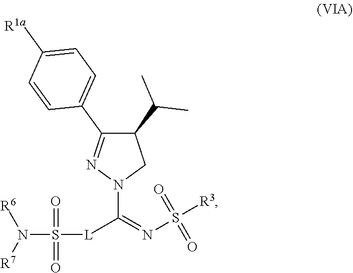
or a pharmaceutically acceptable salt thereof.

49. The compound of claim 43, wherein the compound is a compound of formula (IVB): 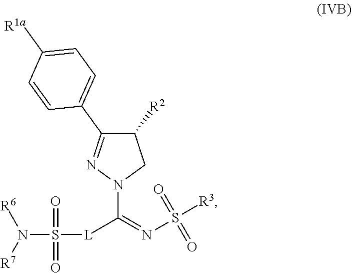
or a pharmaceutically acceptable salt thereof, wherein R^{1a} is halogen, CN, or C₁-C₆ alkoxy.

50. The compound of claim 49, wherein the compound is a compound of formula (VB): 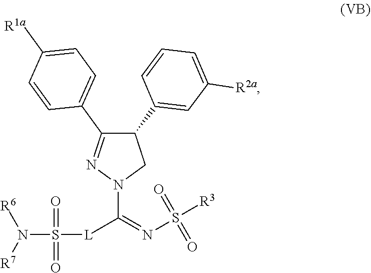
or a pharmaceutically acceptable salt thereof, wherein R^{2a} is H, halogen, C₁-C₆ alkoxy, or CN.

51. The compound of claim 50, wherein R^{2a} is H.

52. The compound of claim 50, wherein R^{2a} is CN.

53. The compound of claim 49, wherein the compound is a compound of formula (VIB): 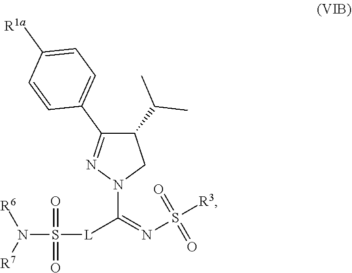
or a pharmaceutically acceptable salt thereof.

54. The compound of any one of claims 44-53, wherein R^{1a} is F.

55. The compound of any one of claims 44-53, wherein R^{1a} is Cl.

56. The compound of any one of claims 44-53, wherein R^{1a} is OCH₃.

57. The compound of any one of claims 43-56, wherein R³ is phenyl substituted with one or more groups selected from halogen, CF₃, CN, C₁-C₆ alkoxy, C₂-C₆ alkynyl, or C(O)NH₂.

58. The compound of claim 57, wherein R³ is:

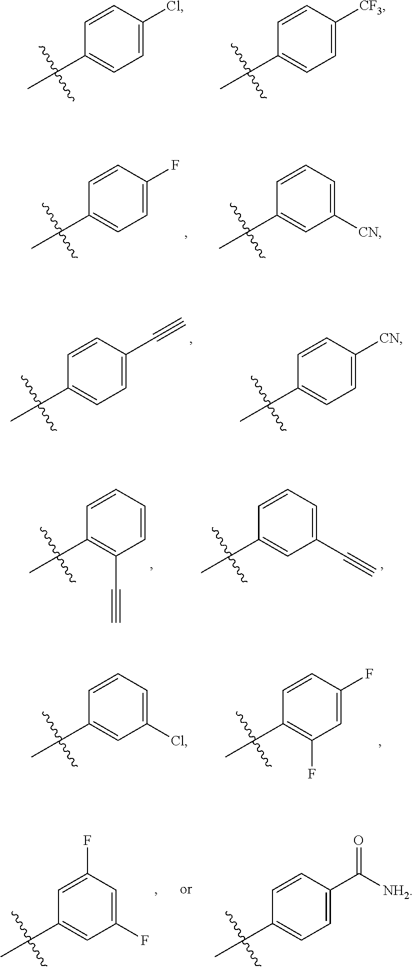

59. The compound of claim 58, wherein R³ is

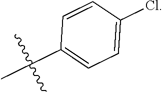 [SMILES: *c1ccc(Cl)cc1]

60. The compound of claim 58, wherein R³ is

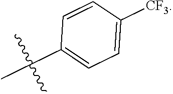 [SMILES: *c1ccc(C(F)(F)F)cc1]

61. The compound of claim 58, wherein R³ is

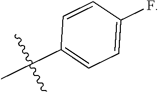 [SMILES: *c1ccc(F)cc1]

62. The compound of any one of claims 43-56, wherein R³ is 5- or 6-membered heteroaryl containing 1-3 nitrogen atoms, wherein the heteroaryl is optionally substituted with C₁-C₆ alkyl.

63. The compound of claim 62, wherein R³ is:

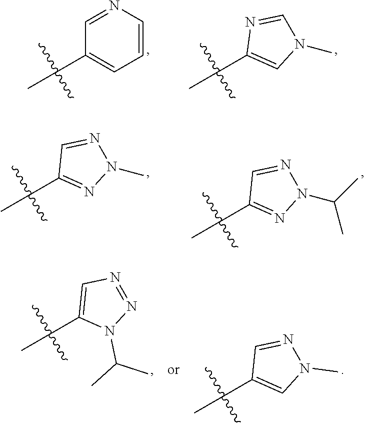

64. The compound of any one of claims 43-63, wherein R⁴ is H.

65. The compound of any one of claims 43-63, wherein R⁴ is C₁-C₆ alkyl.

66. The compound of claim 65, wherein R⁴ is methyl.

67. The compound of claim 65, wherein R⁴ is isopropyl.

68. The compound of any one of claims 43-67, wherein R⁵ is H.

69. The compound of any one of claims 43-67, wherein R⁵ is C₁-C₆ alkyl.

70. The compound of claim 69, wherein R⁵ is methyl.

71. The compound of claim 69, wherein R⁵ is isopropyl.

72. The compound of any one of claims 43-71, wherein R⁶ is H.

73. The compound of any one of claims 43-71, wherein R⁶ is C₁-C₆ alkyl.

74. The compound of claim 73, wherein R⁶ is methyl, ethyl, or isopropyl.

75. The compound of any one of claims 43-71, wherein R⁶ is OH.

76. The compound of any one of claims 43-75, wherein R⁷ is H.

77. The compound of any one of claims 43-75, wherein R⁷ is C₁-C₆ alkyl.

78. The compound of claim 77, wherein R⁷ is methyl, ethyl, or isopropyl.

79. The compound of any one of claims 43-75, wherein R⁷ is OH.

80. The compound of any one of claims 43-71, wherein R⁶ and R⁷, together with the nitrogen atom to which they are attached, form 5- or 6-membered heterocycloalkyl containing 1-2 nitrogen atoms and optionally substituted with C₁-C₆ alkyl.

81. The compound of claim 80, wherein R⁶ and R⁷, together with the nitrogen atom to which they are attached, form:

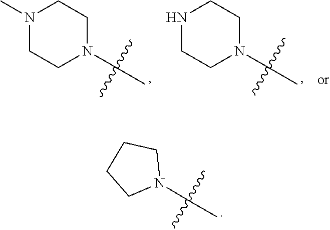

82. The compound of any one of claims 43-81, wherein R⁸ is H.

83. The compound of any one of claims 43-81, wherein R⁸ is CH₃.

84. The compound of claim 43, wherein the compound is selected from any of compounds 99-102, 111-154, 173-174, 183-250, or a pharmaceutically acceptable salt thereof.

85. A compound of formula (III): 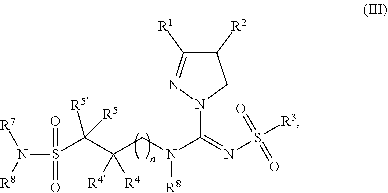
or a pharmaceutically acceptable salt thereof, wherein
R¹ is phenyl optionally substituted with one or more substituents selected from F, Cl, CN, and OCH₃;
R² is C₁-C₆ alkyl, 5- or 6-membered heteroaryl or phenyl optionally substituted with F or CN;
R³ is phenyl substituted with one or two substituents selected from F, Cl, CF₃, CN, OCH₃,C₂-C₆ alkynyl, and C(O)NH₂; or 5- or 6-membered heteroaryl containing 1-3 nitrogen atoms, wherein the heteroaryl is optionally substituted with C₁-C₆ alkyl;
R⁴, R^{4′}, R⁵, and R^{5′} are independently H or C₁-C₆ alkyl;
R⁶ and R⁷ are independently H, OH, or C₁-C₆ alkyl; or
R⁶ and R⁷, together with the nitrogen atom to which they are attached, form 5- or 6-membered heterocycloalkyl containing 1-2 nitrogen atoms and optionally substituted with C₁-C₆ alkyl;
R⁸ is H or CH₃; and
n is 1, 2, or 3.

86. The compound of claim 85, wherein the compound is selected from any of compounds 87-98, or a pharmaceutically acceptable salt thereof.

87. A pharmaceutical composition, comprising a compound of any one of claims 1-86 or a pharmaceutically acceptable salt thereof, and a pharmaceutically acceptable excipient.

88. A method of treating a disease, comprising administering to a subject in need thereof a therapeutically effective amount of a compound of any one of claims 1-86 or a pharmaceutically acceptable salt thereof, wherein the disease is a diabetic disorder, a dyslipidemia disorder, a cardiovascular disorder, an inflammatory disorder, a hepatic disorder, or cancer.

89. The method of claim 88, wherein the disease is a diabetic disorder.

90. The method of claim 89, wherein the diabetic disorder is Type 1 diabetes, Type 2 diabetes, inadequate glucose tolerance, or insulin resistance.

91. The method of claim 88, wherein the disease is a dyslipidemia disorder.

92. The method of claim 91, wherein the dyslipidemia disorder is undesirable blood lipid levels, low levels of high-density lipoprotein, high levels of low-density lipoprotein, high levels of triglycerides, or a combination thereof.

93. The method of claim 88, wherein the disease is a cardiovascular disorder.

94. The method of claim 93, wherein the cardiovascular disorder is atherosclerosis, hypertension, stroke, or heart attack.

95. The method of claim 88, wherein the disease is an inflammatory disorder.

96. The method of claim 95, wherein the inflammatory disorder is osteoarthritis, rheumatoid arthritis, an inflammatory bowel disease, or obesity-associated inflammation.

97. The method of claim 88, wherein the disease is a hepatic disorder.

98. The method of claim 97, wherein the hepatic disorder is liver inflammation, liver fibrosis, non-alcoholic steatohepatitis, fatty liver, enlarged liver, alcoholic liver disease, jaundice, cirrhosis, or hepatitis.

99. The method of claim 88, wherein the disease is cancer.

100. The method of claim 99, wherein the cancer is colon cancer, breast cancer, thyroid cancer, alveolar rhabdomyosarcoma, or hepatocellular carcinoma.

101. A method of treating obesity or a co-morbidity of obesity, comprising administering to a subject in need thereof a therapeutically effective amount of a compound of any one of claims 1-86 or a pharmaceutically acceptable salt thereof.

102. The method of claim 101, wherein the co-morbidity of obesity is diabetes, dyslipidemia, Metabolic Syndrome, dementia, a cardiovascular disease, or a hepatic disease.

103. The method of claim 101, wherein the co-morbidity of obesity is hypertension; gallbladder disease; gastrointestinal disorders; menstrual irregularities; degenerative arthritis; venous statis ulcers; pulmonary hypoventilation syndrome; sleep apnea; snoring; coronary artery disease; arterial sclerotic disease; pseudotumor cerebri; accident proneness; increased risks with surgeries; osteoarthritis; high cholesterol; or increased incidence of malignancy of the ovaries, cervix, uterus, breasts, prostrate, or gallbladder.

104. A method of reversing adipose tissue deposition in a subject, comprising administering to a subject in need thereof a therapeutically effective amount of a compound of any one of claims 1-86 or a pharmaceutically acceptable salt thereof.

105. The method of any one of claims 88-104, further comprising administering to the subject a second therapeutic agent.

106. The method of claim 105, wherein the second therapeutic agent is a PPAR-γ agonist, a biguanide, insulin or an insulin mimetic, a sulfonylurea, an α-glucosidase inhibitor, an HMG-CoA reductase inhibitor, a sequestrant, nicotinyl alcohol, nicotinic acid or a salt thereof, a PPAR-α agonist, an inhibitor of cholesterol absorption, an acyl CoA:cholesterol acyltransferase inhibitor, probucol, a PPAR-α/γ agonist, an ileal bile acid transporter inhibitor, an insulin receptor activator, a dipeptidyl peptidase IV inhibitor, exenatide, pramlintide, an FBPase inhibitor, a glucagon receptor antagonist, glucagon-like peptide 1, a glucagon-like peptide 1 receptor agonist, a growth hormone secretagogue, a growth hormone secretagogue receptor agonist, a growth hormone secretagogue receptor antagonist, a melanocortin agonist, a melanocortin 4 receptor agonist, a beta-3 agonist, a serotonin receptor 2C agonist, an orexin antagonist, a melanin concentrating hormone 1 antagonist, a melanin concentrating hormone 2 agonist, a melanin concentrating hormone 2 antagonist, a galanin antagonist, a CCK agonist, a CCK-A agonist, a corticotropin-releasing hormone agonist, an NPY 5 antagonist, an NPY 1 antagonist, a histamine receptor-3 modulator, a histamine receptor-3 blocker, a p-hydroxy steroid dehydrogenase-1 inhibitor, a phosphodiesterase inhibitor, a phosphodiesterase-3B inhibitor, a norepinephrine transport inhibitor, a non-selective serotonin/norepinephrine transport inhibitor, a ghrelin antagonist, a leptin derivative, a bombesin receptor subtype 3 agonist, a ciliary neurotrophic factor or a derivative thereof, a monoamine reuptake inhibitor, an uncoupling protein-1 activator, an uncoupling protein-2 activator, an uncoupling protein-3 activator, a thyroid hormone beta agonist, a fatty acid synthase inhibitor, a diacylglycerol acetyltransferase 2 inhibitor, an acetyl-CoA carboxylase-2 inhibitor, a glucocorticoid antagonist, an acyl-estrogen, a lipase inhibitor, a fatty acid transporter inhibitor, a dicarboxylate transporter inhibitor, a glucose transporter inhibitor, a sodium-glucose co-transporter, a phosphate transporter inhibitor, a serotonin reuptake inhibitor, a thiazolidinedione, Metformin, Topiramate, an opiate antagonist, a non-selective transport inhibitor, or a MAO inhibitor.

107. The method of claim 106, wherein the second therapeutic agent is a glucagon-like peptide 1 receptor agonist.

108. The method of claim 107, wherein the second therapeutic agent is liraglutide, semaglutide, exenatide, lixisenatide, dulaglutide, ortirzepatide.

109. The method of claim 108, wherein the second therapeutic agent is semaglutide.

110. The method of claim 108, wherein the second therapeutic agent is tirzepatide.

111. The method of any one of claims 88-110, where the subject is a human.

112. The method of any one of claims 88-110, wherein the subject is a non-human mammal.

113. Use of a compound of any one of claims 1-86 for the manufacture of a medicament for use in a method of any one of claims 88-112.

114. The compound of any one of claims 1-86 for use in a method of any one of claims 88-112.
